(19) United States
(12) Patent Application Publication
(10) Pub. No.: US 2004/0171074 A1
(43) Pub. Date: Sep. 2, 2004

(54) Structures of substrate binding pockets of SCF complexes

(75) Inventors: Stephen Orlicky, Toronto (CA); Frank Sicheri, Toronto (CA); Mike Tyers, East York (CA); Andrew Willems, Toronto (CA); XiaoJing Tang, North York (CA)

Correspondence Address: MERCHANT & GOULD PC, P.O. BOX 2903, MINNEAPOLIS, MN, 55402-0903, US

(73) Assignee: MOUNT SINAI HOSPITAL, TORONTO (CA)

(21) Appl. No.: 10/687,732

(22) Filed: Oct. 17, 2003

Related U.S. Application Data
(60) Provisional application No. 60/419,606, filed on Oct. 17, 2002.

(51) Int. Cl. G01N 33/53; G06F 19/00; G01N 33/48; G01N 33/50

(52) U.S. Cl. 435/7.1; 702/19; 530/350

(57) ABSTRACT

The present invention relates to binding pockets of Skp1-Cdc53/Cullin-F-box protein (SCF) E3 ubiquitin ligases associated with substrate selection and/or orientation. In particular, the invention relates to a crystal comprising such binding pockets. The crystal may be useful for modeling and/or synthesizing mimetics of a binding pocket or ligands that associate with the binding pocket. Such mimetics or ligands may be capable of acting as modulators of the interactions of a SCF E3 ubiquitin ligase and its substrates, and they may be useful for treating, inhibiting, or preventing diseases modulated by such interactions. Methods are also provided for regulating a SCF E3 ubiquitin ligase comprising changing a binding pocket associated with substrate selection and/or orientation.

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to binding pockets of Skp1-Cdc53/Cullin-F-box protein (SCF) E3 ubiquitin ligases associated with substrate selection and/or orientation. In particular, the invention relates to a crystal comprising such binding pockets. The crystal may be useful for modeling and/or synthesizing mimetics of a binding pocket or ligands that associate with the binding pocket. Such mimetics or ligands may be capable of acting as modulators of the interactions of an SCF E3 ubiquitin ligase and its substrates, and they may be useful for treating, inhibiting, or preventing diseases modulated by such interactions.

Methods are also provided for regulating an SCF E3 ubiquitin ligase comprising changing a binding pocket associated with substrate selection and/or orientation.

### BACKGROUND

The ubiquitin proteolytic system controls the precisely timed degradation of regulatory proteins in signaling, development and cell cycle progression. Substrate ubiquitination is catalyzed by a cascade of enzymes, termed E1, E2 and E3, which activate and then conjugate ubiquitin to the substrate (Hershko and Ciechanover, 1998). E3 enzymes, also known as ubiquitin ligases, contain substrate-specific recognition domains and catalyze the final step in ubiquitin transfer. Recognition is mediated by primary sequence elements in the substrate, referred to as degrons (Varshavsky, 1991). Control of the E3-substrate interaction forms the basis for regulated proteolysis; often post-translational substrate modification, most commonly phosphorylation, serves to target substrates to their cognate E3 enzymes (Deshaies, 1999). Two main classes of E3 enzyme are now evident, as characterized by the presence of either a HECT domain or a RING domain. The HECT domain class forms a catalytically essential thioester with ubiquitin, whereas the RING domain class relies on the E2 enzymes to provide catalytic activity (Pickart, 2001). The RING domain forms an E2 docking site and orients the substrate with respect to the E2.

Phosphorylation-dependent degrons direct substrates to a recently described class of multisubunit E3 enzymes termed Skp1-Cdc53/Cullin-F-box protein (SCF) complexes. SCF complexes are built on an invariant core machinery comprised of the adapter protein Skp1, the scaffold protein Cdc53 (called Cull in metazoans), and the RING-H2 domain protein Rbx1 (also called Roc1 or Hrt1), which interacts with an E2 enzyme, usually Cdc34 (Pickart, 2001). Substrates are brought to the core complex by one of a large family of variable adapter subunits called F-box proteins, each of which targets a limited number of specific substrates (Bai et al., 1996, Patton et al., 1998). F-box proteins typically have a bipartite structure with an N-terminal ˜40 amino acid F-box motif and a C-terminal protein-protein interaction domain, such as WD40 repeats or leucine rich repeats, which bind substrates (Bai et al, 1996; Feldman et al, 1997; Skowyra et al., 1997). The overall architecture of SCF complexes is conserved in several related ubiquitin ligase complexes including the Anaphase Promoting Complex/Cyclosome and the Von Hippel Lindau (VHL) tumor suppressor protein complex, each of which contain cullin family members, RING-H2 domain and substrate recognition subunits (Pickart, 2001; Kaelin, 2002).

Cell cycle progression depends on the precisely timed elimination of cyclins and cyclin-dependent kinase (CDK) inhibitors by the ubiquitin system (Harper et al, 2002). In yeast, G1 cyclin CDK activity phosphorylates a CDK inhibitor called Sic1, whose degradation is necessary for onset of B-type cyclin CDK activity and DNA replication (Schwob et al., 1994). Phospho-Sic1 is specifically recognized by the F-box protein Cdc4, which recruits Sic1 for ubiquitination by the Cdc34-SCF complex (Bai et al., 1996; Feldman et al., 1997; Skowyra et al., 1997). Stable forms of Sic1 that lack CDK phosphorylation sites cause a G1 phase arrest (Verma et al., 1997), whereas deletion of SIC1 causes premature DNA replication and rampant genome instability (Lengronne and Schwob, 2002). Cdc4 recruits several other substrates to the SCF core complex in a phosphorylation dependent manner, including the Cln-Cdc28 inhibitor/cytoskeletal scaffold protein Far1, the replication protein Cdc6 and the transcription factor Gcn4 (Patton et al., 1998). The F-box protein Grr1 functions in an analogous manner to render G1 cyclins unstable throughout the cell cycle, in a manner that depends on recognition of phospho-epitopes by the LRR domain of Grr1 (Skowyra et al, 1997; Hsiung et al, 2001).

In the metazoan cell cycle, SCF complexes target phosphorylated forms of the CDK inhibitor p27^{Kip1} and cyclin E, among other substrates. Interestingly, F-box protein specificity for these substrates is reversed compared to yeast, in that the WD40 domain of hCdc4/Fbw7/Ago/SEL-10 recognizes cyclin E (Strohmaier et al., 2001; Koepp et al., 2001; Moberg et al., 2001), whereas the LRR domain of Skp2 recognizes p27^{Kip1} in conjunction with the CDK-binding protein Cks1 (Harper, 2001). Both of these degradation pathways are perturbed in cancer cells. Many primary tumors express high levels of Skp2, which leads to premature degradation of p27^{Kip1} and cell cycle entry (Harper, 2001). Conversely, loss of Cdc4 function causes deregulation of cyclin E-CDK2 activity, which leads to precocious S phase entry and genome instability (Spruck et al., 1999). Mutations in the Drosophila homolog of CDC4, called ago, were isolated as homozygous recessive alleles in a screen for excess cell proliferation, a defect attributed to ectopic cyclin E activity (Moberg et al., 2001). Mutations in hCDC4 have been detected in several cancer cell lines that exhibit high levels of cyclin E (Moberg et al., 2001; Strohmaier et al., 2001), as well as in a significant fraction of primary endometrial cancers (Spruck et al., 2002). In addition, hCDC4 is located in the 4q32region, which is often deleted in various cancers (Spruck et al., 2002). Significantly, a high level of cyclin E correlates strongly with low survival rates in breast cancer (Keyomarsi et al., 2002). Other important substrates appear to be targeted for degradation by Cdc4 orthologs in a phosphorylation-dependent manner, including actived forms of the developmental regulator Notch and the presenilins, which are implicated in familial early onset Alzheimer's disease (Lai, 2002; Selkoe, 2001). SCF-dependent proteolysis also mediates other important signaling events, including phosphorylation-dependent degradation of the NFκB inhibitor IκBα and the proto-oncogene product β-catenin by the F-box protein β-TrCP (Pickart, 2001).

Several F-box proteins can recognize short phosphopeptide motifs that correspond to substrate sequences. However, it is unknown whether such interactions are analogous to phosphorylation-dependent interactions of SH2, PTB, 14-3-3, WW and FHA domains, each of which has been crystallized with its cognate phosphopeptide (Yaffe and Elia, 2001). For many SCF substrates, including Sic1, Cdc6 and Cln2, phosphorylation on multiple dispersed sites is required for recognition and degradation (Patton et al., 1998). We recently defined a high affinity consensus phosphopeptide binding motif for Cdc4, termed the Cdc4 phospho-degron (CPD), which bears the consensus I/L-I/L/P-pT-P-<KR>₄ [SEQ ID NO:1], where < > indicates a disallowed residue (Nash et al., 2001). The P0 phospho-threonine residue, or less favorably a phospho-serine residue, and the P+1 proline are essential for interaction with Cdc4. Unexpectedly, the CPD consensus is at odds with the CDK phosphorylation site consensus, S/T-P-X-K/R [SEQ ID NO:2](Endicott et al., 1999). Thus, substrate recognition by the targeting kinase is counter-balanced against the targeting component of the degradation machinery. All nine CPD sites in Sic1 have one or more sub-optimal features: all lack consensus hydrophobic residues in the P−1 or P−2 positions, four have serine in place of threonine in the P0 position, and seven contain a disfavored basic residue in one of the +2 to +5 positions. Unexpectedly, Sic1 must be phosphorylated on at least six of its nine sites in order to allow recognition by Cdc4 (Nash et al., 2001). This requirement for multi-site phosphorylation in principle renders the rate of Sic1 degradation proportional to the sixth power of G1 CDK concentration (Ferrell, 1996). The inherently ultrasensitive nature of the Sic1 degradation reaction appears critical for the coordinated initiation of DNA replication by S phase CDK activity (Nash et al., 2001; Lengronne and Schwob, 2002).

The mechanism of the ubiquitin conjugation reaction is not well understood. The ability of E2-E3 enzyme complexes to form polymers of ubiquitin, itself an 8 kDa protein, on a protein substrate presumably demands a large catalytic cradle simply to accommodate the initial reactants (Pickart, 2001). The sequential addition of ubiquitin moieties onto the substrate must also entail considerable flexibility of the substrate and/or the enzyme complex in order to extend the ubiquitin chain. Recent structure determination and modeling of three E2-E3 complexes has provided insight into these issues. A complex of the E2 enzyme UbCH7 and the HECT domain enzyme E6AP reveals a distance of ˜50 Å between the E2 and E3 active sites, suggesting that catalytic transfer of ubiquitin requires large scale movements in an as yet undefined process (Huang et al., 1999). Similarly, a complex between UbCH7 and the RING domain E3 c-Cb1 contains a substantial gap between the E2 active site and the substrate binding site on c-Cb1 (Zheng et al., 2000). Structures of the SOCS-box adapter protein VHL in complex with a hydroxylated substrate peptide have recently been solved (Kaelin, 2002), but the orientation of the substrate binding site with respect to the E2 enzyme is unknown. Finally, structure determination and molecular modeling of the holo-SCF^{skp2} complex again suggests a distance of ˜50 Å between the substrate binding LRR domain in Skp2 and the E2 active site (Zheng et al., 2002). Notably, the extensive interdigitation of the Skp1-Skp2 interface and the Skp2 inter-domain interface rigidly fixes the orientation of the LRRs of Skp2, suggesting that the F-box protein might hold the substrate in a very precise orientation with respect to the E2 enzyme (Schulman et al., 2000). However, because the substrate binding site on Skp2 has not been determined, either by mutation or by co-crystallization with substrate peptide, it is not possible to deduce how SCF substrates might be positioned with respect to the E2 catalytic site.

### SUMMARY OF THE INVENTION

Applicants have determined the structures of binding pockets of SCF E3 ubiquitin ligases involved in substrate recognition and/or orientation. More particularly, Applicants have solved the x-ray crystal structure of binding pockets of F-box proteins/F-box protein-Skp1 complexes of SCF E3 ubiquitin ligases that interact with Cdc4 phospho-degron (CPD) motifs.

Solving the crystal structure has enabled the determination of key structural features of substrate binding pockets of a SCF E3 ubiquitin ligase, particularly the shape of binding pockets, or parts thereof, that permit association of a substrate with a SCF E3 ubiquitin ligase or part thereof. The crystal structure also enables the determination of key structural features in substrates or ligands that interact or associate with the binding pockets.

Knowledge of the structural features of substrate binding pockets of a SCF E3 ubiquitin ligase is of significant utility in drug discovery. The SCF E3 ubiquitin ligase substrate interaction is the basis of many biological mechanisms. In particular it is the basis for regulated ubiquitin proteolysis resulting in degradation of regulatory proteins involved in signaling, development, and cell cycle progression. In addition, drugs may exert their effects through association with the binding pockets of SCF E3 ubiquitin ligases. The associations may occur with all or any parts of a binding pocket. An understanding of the association of a drug with binding pockets of SCF E3 ubiquitin ligases will lead to the design and optimization of drugs having more favorable associations with their targets and thus provide improved biological effects. Therefore, information about the shape and structure of substrate binding pockets of SCF E3 ubiquitin ligases is invaluable in designing potential modulators of the SCF E3 ubiquitin ligases for use in treating diseases and conditions associated with or modulated by the SCF ubiquitin ligases, including cancer and Alzheimer's Disease.

The present invention relates to an isolated binding pocket of an SCF E3 ubiquitin ligase involved in substrate recognition and/or orientation. In an embodiment, the invention relates to a binding pocket of an F-box protein/F-box protein-Skp1 complex of a SCF E3 ubiquitin ligase that interacts with a Cdc4 phospho-degron (CPD) motif. In an aspect of the invention, the binding pocket regulates the binding of a CPD motif to a SCF E3 ubiquitin ligase.

In an embodiment, the invention comprises the structure of a WD repeat domain of an F-box protein. The structure may also comprise a helical linker of an F-box protein and optionally an F-box domain of an F-box protein. Still further the structure may comprise a Skp1 protein.

The invention also relates to a crystal comprising a binding pocket of a SCF E3 ubiquitin ligase involved in substrate recognition and/or orientation.

In an embodiment, the invention provides a crystal comprising a WD repeat domain of an F-box protein. The crystal may also comprise a helical linker of an F-box protein and optionally an F-box domain of an F-box protein. Still further the crystal may comprise a Skp1 protein.

The present invention also contemplates molecules or molecular complexes that comprise all or parts of either one or more binding pockets of the invention, or homologs of these binding pockets that have similar structure and shape.

The invention also contemplates a crystal comprising a binding pocket of a SCF E3 ubiquitin ligase involved with substrate recognition and/or orientation in association with a substrate (e.g. CPD motif). A substrate may be complexed or associated with a binding pocket. The invention further contemplates a crystal comprising a binding pocket of a SCF E3 ubiquitin ligase involved with substrate recognition and/or orientation in association with a ligand. A ligand may be a modulator of the activity of a SCF E3 ubiquitin ligase. A ligand may be complexed or associated with a binding pocket

In an aspect the invention contemplates a crystal comprising a binding pocket of an SCF E3 ubiquitin ligase involved in substrate recognition and/or orientation complexed with a substrate from which it is possible to derive structural data for the substrate.

The shape and structure of a binding pocket may be defined by selected atomic contacts in the pocket. In an embodiment, the binding pocket is defined by one or more atomic interactions or enzyme atomic contacts as set forth in Table 3 or Table 4. Each of the atomic interactions is defined in Table 3 or Table 4 by an atomic contact (more preferably, a specific atom where indicated) on the F-box protein and by an atomic contact (more preferably a specific atom where indicated) on the substrate. The atomic interactions are also defined by an atomic contact on one portion of the F-box protein and an atomic contact on another portion of the F-box protein.

An isolated polypeptide comprising a binding pocket with the shape and structure of a binding pocket described herein is also within the scope of the invention.

The invention also provides a method for preparing a crystal of the invention, preferably a crystal of a binding pocket of an SCF E3 ubiquitin ligase involved in substrate recognition and/or orientation, or a complex of such a binding pocket and a substrate.

Crystal structures of the invention enable a model to be produced for a binding pocket of the invention, or complexes or parts thereof. The models will provide structural information about the interactions of a substrate or ligand with a binding pocket. Models may also be produced for substrates and ligands. A model and/or the crystal structure of the present invention may be stored on a computer-readable medium.

The present invention includes a model of a binding pocket of the present invention that substantially represents the structural coordinates specified in Table 6 or portions thereof. The invention also includes a model that comprises modifications of the structure substantially represented by the structural coordinates specified in Table 6. A model is a representation or image that predicts the actual structure of the binding pocket. As such, a model is a tool that can be used to probe the relationship between a binding pocket's structure and function at the atomic level, and to design molecules that can modulate the binding site and accordingly activity of an F-box protein or SCF complex.

Thus, the invention provides a model of: (a) a binding pocket of an SCF E3 ubiquitin ligase involved in substrate recognition and/or orientation; and (b) a modification of the model of (a).

A method is also provided for producing a model of the invention representing a binding pocket of an SCF E3 ubiquitin ligase involved in substrate recognition and/or orientation, comprising representing amino acids of the binding pocket at substantially the structural coordinates specified in Table 6.

A crystal and/or model of the invention may be used in a method of determining the secondary and/or tertiary structures of a polypeptide or binding pocket with incompletely characterised structure. Thus, a method is provided for determining at least a portion of the secondary and/or tertiary structure of molecules or molecular complexes which contain at least some structurally similar features to a binding pocket of the invention. This is achieved by using at least some of the structural coordinates set out in Table 6.

A crystal of the invention may be useful for designing, modeling, identifying, evaluating, and/or synthesizing mimetics of a binding pocket or ligands or substrates that associate with a binding pocket. Such mimetics or ligands may be capable of acting as modulators of an F-box protein or SCF E3 ubiquitin ligase activity, and they may be useful for treating, inhibiting, or preventing diseases modulated by such a protein or ligase.

Thus, the present invention contemplates a method of identifying a modulator of a F-box protein or an SCF E3 ubiquitin ligase comprising the step of applying the structural coordinates of a binding pocket, or atomic interactions, or atomic contacts of a binding pocket, to computationally evaluate a test ligand or substrate for its ability to associate with the binding pocket, or part thereof. Use of the structural coordinates of a binding pocket, or atomic interactions, or atomic contacts of a binding pocket to design or identify a modulator is also provided.

In an embodiment, the invention contemplates a method of identifying a modulator of an F-box protein or an SCF E3 ubiquitin ligase comprising determining if a test agent inhibits or potentiates the interaction of an F-box protein or SCF E3 ubiquitin ligase with its substrate.

The invention further contemplates classes of modulators of F-box proteins or SCF E3 ubiquitin ligases based on the shape and structure of a ligand or substrate defined in relation to the molecule's spatial association with a binding pocket of the invention. Generally, a method is provided for designing potential inhibitors of an F-box protein-substrate interaction or SCF E3 ubiquitin ligase-substrate interaction comprising the step of applying the structural coordinates of a substrate or ligand defined in relation to its spatial association with a binding pocket, or a part thereof, to generate a compound that is capable of associating with the binding pocket.

It will be appreciated that a modulator of an F-box protein or SCF E3 ubiquitin ligase may be identified by generating an actual secondary or three-dimensional model of a binding pocket, synthesizing a compound, and examining the components to find whether the required interaction occurs.

A potential modulator of an F-box protein or SCF E3 ubiquitin ligase identified by a method of the present invention may be confirmed as a modulator by synthesizing the compound, and testing its effect on the F-box protein or SCF E3 ubiquitin ligase in an assay.

A modulator of the invention may be converted using customary methods into pharmaceutical compositions. A modulator may be formulated into a pharmaceutical composition containing a modulator either alone or together with other active substances.

Therefore, the methods of the invention for identifying modulators may comprise one or more of the following additional steps:

(a) testing whether the modulator is a modulator of the activity of an F-box protein or an SCF E3 ubiquitin ligase, preferably testing the activity of the modulator in cellular assays and animal model assays;

(b) modifying the modulator;

(c) optionally rerunning steps (a) or (b); and

(d) preparing a pharmaceutical composition comprising the modulator.

Steps (a), (b) (c) and (d) may be carried out in any order, at different points in time, and they need not be sequential.

Still another aspect of the present invention provides a method of conducting a drug discovery business comprising:

(a) providing one or more systems employing the atomic interactions, atomic contacts, or structural coordinates of a binding pocket of an F-box protein or SCF E3 ubiquitin ligase involved in substrate recognition and/or orientation, for identifying agents by their ability to inhibit or potentiate the atomic interactions or atomic contacts of a binding pocket;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

A further aspect of the present invention provides a method of conducting a drug discovery business comprising:

(a) providing one or more systems for identifying agents by their ability to inhibit or potentiate the interaction between an F-box protein or SCF complex and its substrate; and

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject methods can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:

(a) providing one or more systems employing the atomic interactions, atomic contacts, or structural coordinates of a binding pocket of an F-box protein or SCF complex involved in substrate recognition and/or orientation, for identifying agents by their ability to inhibit or potentiate the atomic interactions or atomic contacts;

(b) (optionally) conducting therapeutic profiling of agents identified in step (a) for efficacy and toxicity in animals; and

(c) licensing, to a third party, the rights for further drug development and/or sales for agents identified in step (a), or analogs thereof.

Methods are also provided for regulating an F-box protein—substrate interaction or an SCF E3 ubiquitin ligase-substrate interaction by changing a binding pocket involved in substrate recognition and/or orientation. A binding pocket may be changed by altering amino acid residues forming the binding pocket (e.g. introducing mutations) or using a modulator.

The invention also contemplates a method of treating or preventing a condition or disease associated with an F-box protein or an SCF E3 ubiquitin ligase in a cellular organism, comprising:

(a) administering a modulator of the invention in an acceptable pharmaceutical preparation; and

(b) potentiating or inhibiting the F-box protein or SCF E3 ubiquitin ligase to treat or prevent the disease.

In an embodiment the condition or disease is cancer or Alzheimer's disease.

The invention provides for the use of a modulator identified by the methods of the invention in the preparation of a medicament to treat or prevent a disease in a cellular organism. Use of modulators of the invention to manufacture a medicament is also provided.

These and other aspects of the present invention will become evident upon reference to the following detailed description and Tables, and attached drawings.

### DESCRIPTION OF THE DRAWINGS AND TABLES

The present invention will now be described only by way of example, in which reference will be made to the following Figures:

FIG. 1 shows structure based sequence alignments of (A) Skp1 orthologs and (B) Cdc4 orthologs (red) and paralogs (black). Human Fbw7 and P-TrCP1 are isoforms 1 and 2, respectively. Secondary structure elements are colored as in FIG. 2A. Disordered regions in the crystal structure are shown as dashed lines. Red residues are essential for the Cdc4 function, blue residues strongly influence but do not abrogate function, green residues are non-essential but conserved around the binding pocket, and yellow residues are conserved elsewhere. Circles indicate mutations associated with excessive cell proliferation in flies and/or cancer in humans. Deletion of residues 37-64 in Skp1 is denoted by a triangle and a replacement of two closely placed loops from residues 602-605 and 609-624 is denoted by the underline of the short interloop sequence Gly-Glu-Leu. Insertions to optimize sequence alignments are indicated by number of residues inserted in gray. The non-standard β-strand element 9¹ in ScCdc4 is marked by the red asterisk and is shown in full at the bottom of the alignment. Residues that anchor helix α6 to the F-box domain are marked by green hearts, those that anchor helix α6 to the WD40 domain by red hearts and those that make direct contact between the WD40 domain and F-box domain by blue asterisks. [SEQ ID NOs 3-16.]

FIG. 2 shows an overview of the Skp1-Cdc4-CPD complex. (A) Ribbon representation of Skp1 and the F-box domain (274-319), the helical linker region (331-366), and the WD40 domain of Cdc4 (367-744) coloured green, red, pink, and blue, respectively. The bound cyclin E derived CPD peptide is shown in purple with the phosphothreonine moiety shown in ball and stick representation. Secondary structure elements are indicated. Positions of disordered loop regions are shown as ribbon breaks. All ribbons representations were generated using Ribbons. (B) Ribbons representation highlighting the WD 40 domain of Cdc4. β propeller blades are denoted PB1 to PB8, and the component secondary structure elements are indicated. Ribbons and CPD peptide are coloured as in (A). Position of the WD40 domain is identical to that in FIGS. 4A to 4C. (C) The structured linkage between the WD40 domain and the F box domain of Cdc4.

FIG. 3 shows an overview of the CPD binding region of the Ccdc4 WD40 repeat domain. (A) Molecular surface representation of the CPD binding pocket indicating invariant and highly conserved residues. Basic, hydrophobic and small residues are coloured blue, green and orange respectively. The bound CPD is shown in ball and stick representation with carbon, nitrogen, oxygen and phosphorous atoms coloured white, blue, red and yellow respectively. All surface representation were generated using Grasp. (B) Surface representation of CPD binding region as oriented in (A) coloured according to electrostatic potential. Blue and red indicate regions of positive and negative potential respectively (10 to −10 kBT). Residues of the bound CPD are labeled. (C) Stereo ribbons representation highlighting side chains and molecular interactions in the CPD binding pocket. CPD residues and highly conserved and invariant Cdc4 residues are displayed in ball and stick representation. Sites of mutation that give rise to severe loss of function are coloured red, and intermediate loss of function are coloured yellow (see Table 5). All other highly conserved and invariant residues are coloured green. Reference propeller blades of the WD40 repeat domain are indicated. (D) Stereo ribbons representation of the CPD binding pocket highlighting cancer causing mutations in drosophila and human Cdc4 orthologues. Arginine mutations in H-cell lines or entrometrial cells are coloured red. Drosophila mutations are coloured blue and Cdc4 temperature sensitive mutations (Rosamond personal communication) are coloured yellow. (E) Multiple Anomalous Dispersion phased electron density map corresponding to the CPD bound to the WD40 repeat domain of Cdc4. Refined CPD model is shown in ball and stick representation. Figure generate using O. (F) Schematic of CPD binding pocket interactions with the CPD peptide.

FIG. 4 shows (A) Stereo ribbons representation of the human Skp1-Skp2 complex superimposed on the yeast Skp1-Cdc4-CPD complex. Human Skp1-Skp2 and yeast Skp1-Cdc4 were superimposed through a least squares optimization of Skp1, strands 1 to 3 and a-helices 1 to 6 (RMSD=0.74 Å). The yeast Skp1-Cdc4 complex is coloured as in FIG. 2. Human Skp1, the Skp2 F-box, and the Skp2 Leucine-rich repeat domain are coloured orange, green, and light blue, respectively. Skp1 and F box secondary structure elements that deviate significantly in size and position between the two structures are labeled. (B) Model of the SCFCdc4-CPD E2 complex. The yeast Skp1-Cdc4-CPD complex is coloured as in FIG. 2. Cull, Rbx1, and E2 proteins are coloured pink, red, and light blue, respectively. The arrow indicates the distance between the peptide binding site and the active site cysteine of the E2. The structure was generated using the ternary complex of the cullin cdc53, rbx1, Skp1, previously reported, and superimposing the E2 structure from the E2/Cb1 ring finger structure and the structure of Skp1, Cdc4 and a phosphorylated CPD peptide

FIG. 5 shows (A) Selection of Sic1 phosphoisoforms by wild type and mutant forms of Cdc4. (B) In vitro ubiquitination of Sic1 isoforms by wild type and mutant SCFCdc4 complexes. (C) Natural CPD sites deviate from the optimal CPD by one or more or more residues.

FIG. 6 shows substrate orientation within the Skp1-Cdc4-CPD complex. (A) Comparison of the ScSkp1-ScCdc4-CPD complex and the hSkp1-hSkp2 complex. Complexes were superimposed through a least squares optimization of Skp1 β-strands 1 to 3 and a-helices 1 to 6 (RMSD Cα=0.74 Å). Skp1 and F-box secondary structure elements that deviate significantly in size and position between the two structures are labeled. (B) Model of the ubiquitin-E2-SCF^{Cd4}-CPD complex. The arrow indicates the 59 Å distance separating the phosphate group of the CPD and the active site cysteine of the E2.

FIG. 7 shows the CPD binding pocket of the WD40 domain. (A) Surface representation of the CPD binding pocket indicating invariant and highly conserved residues. Basic (blue), hydrophobic (green) and small polar residues (orange) are shown. The bound CPD is in ball and stick representation with carbon (white), nitrogen (blue), oxygen (red) and phosphorous (yellow) atoms shown. (B) Surface representation of CPD binding region indicating electrostatic potential. Blue and red indicate regions of positive and negative potential, respectively, over the range 10 to −10 kBT. (C) Stereo ribbons representation of side chains and molecular interactions in the CPD binding pocket. Highly conserved and invariant side chains of Cdc4 and the CPD are displayed in ball and stick representation. Sites of mutation that give rise to severe and intermediate loss of function (see FIG. 8) are colored red and blue, respectively; non-essential residues are colored green.

(D) Schematic of CPD Binding Pocket Interactions With the CPD Peptide.

FIG. 8 shows structure-guided mutational analysis of Cdc4. (A) Residues required for interaction of phospho-Sic1 and Cdc4 in vitro. Sic1 was phosphorylated with Cln2-Cdc28 kinase and captured onto resin loaded with either wild type or the indicated mutant forms of Skp1-Cdc4 complex. (B) Residues essential for Cdc4 function in vivo. Complementation of a cdc4Δ strain by the indicated alleles was assessed in a plasmid shuffle assay. The R485A, R467A and R534A mutations in Cdc4 have been previously shown to disrupt function in vivo (Nash et al., 2001) and so are not shown. (C) Effect of Cdc4 mutations on sensitivity to increased SIC1 dosage. Strains bearing indicated CDC4 alleles were tested for sensitivity to overexpression of wild type SIC1 and a partially stabilized version, SIC1^{Thr33Val} from the GAL1 promoter. Strains were incubated on galactose or glucose medium for 2 days at 30° C.

FIG. 9 shows the modulation of the multisite requirement for phospho-Sic1-Cdc4 interaction. (A) All natural CPD sites in Sic1 deviate from the CPD consensus. Underlined residues indicate sub-optimal residues at the P−1 and P−2 positions, boxed residues indicate sub-optimal basic residues at the P+2 to P+5 positions and asterisks indicate a sub-optimal pSer at the P0 position. (B) Capture of Sic1 phospho-isoforms by wild type and mutant Cdc4. Pools of differentially phosphorylated Sic1 were captured on Skp1-Cdc4 resin, using either wild type or the indicated mutant forms of Cdc4 compromised for selection at the P−1 position (V384N W717N) or the P+2 to P+5 positions (K402A R443D). The input and bound phospho-Sic1 isoform pools were resolved by denaturing IEF-2D gel electrophoresis and visualized by anti-Sic1 immunoblot. (C) Ubiquitination of phospho-Sic1 isoforms by wild type and mutant SCF^{Cdc4} complexes. Pools of differently phosphorylated Sic1 were incubated in solution with an equimolar amount of the indicated SCFCdC4 complexes, Cdc34, ubiquitin and ATP for 1 h at 30° C. Input and reaction products were separated and visualized as in (B). Arrows indicate the less phosphorylated forms of Sic1 captured by Cdc4 selection mutants. Asterisk indicates more extensively ubiquitinated species (D) Possible interaction mechanisms for single site and multi-site dependent substrate binding to Cdc4. In a two-site cooperative interaction model (left), a primary high affinity CPD binding site acts in conjunction with a secondary weak CPD binding site. The free energy for the two interactions is additive and so the overall K_{d} increases multiplicatively. In a single-site allovalent interaction (right), multiple low affinity CPD sites engage a single CPD binding site on Cdc4 in equilibrium. The high local concentration of CPD sites increases the probability of binding such that Sic1 is unable to diffuse away from Cdc4 before re-binding occurs. The probability of re-binding increases as an exponential function of the number of CPD sites, thus accounting for the apparent cooperativity of the interaction.

The present invention will now be described only by way of example, in which reference will be made to the following Tables:

Table 1 shows data collection, structure determination and refinement statistics of a crystal of the invention.

Table 2 shows data collection, structure determination and refinement statistics of a crystal of the invention.

Table 3 shows intermolecular contacts in a binding pocket of the invention.

Table 4 shows intermolecular contacts in a binding pocket of the invention.

Table 5 shows mutant cdc4 polyppeptides of the invention. Mutational analysis of the CPD binding surface. Mutants were tested in vitro by ability to bind phosphorylated Sic1 and then captured onto GST-Skp1/Cdc4 resin and detected with anti-sic1 antibody. Mutants were tested in vivo by ability to degrade GAL1-SIC1 or various phosphorylation mutants. Sites are as follows: 3=Thr 33, Thr 45, Ser 76; 4=Thr 5, Thr 33, Thr 45, Ser 76; 5=Thr 2, Thr 5, Thr 33, Thr 45, Ser 76; 6=Thr 2, Thr 5, Thr 33, Thr 45, Ser 69, Ser 76; 7=Thr 2, Thr 5, Thr 33, Thr 45, Ser 69, Ser 76, Ser 80. GAL1-SIC1 plasmids were transformed into a cdc4Δ strain containing a copy of CDC4 on a TRP1 ARS CEN plasmid. Strains were incubated for 2 days at 30° C.

Table 6 shows the structural coordinates of a binding pocket of the invention.

In Table 6, from the left, the second column identifies the atom number; the third identifies the atom type; the fourth identifies the amino acid type; the sixth identifies the residue number; the seventh identifies the x coordinates; the eighth identifies the y coordinates; the ninth identifies the z coordinates; the tenth identifies the occupancy; and the eleventh identifies the temperature factor.

Table 7 lists the oligonucleotides used in the studies described in the examples.

Table 8 lists the plasmids used in the studies described in the examples.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

Abbreviations for amino acid residues are the standard 3-letter and/or 1-letter codes used in the art to refer to one of the 20 common L-amino acids. Likewise abbreviations for nucleic acids are the standard codes used in the art.

“Skp1-Cdc53/Cullin-F-box protein (SCF) E3 ubiquitin ligases” or “SCF complex” refers to a protein complex comprising the adaptor protein Skp1, the scaffold protein cdc53/cullin, a RING-H2 domain protein Rbx1 (also called Roc1 or Hrt1), and an F-box protein, which protein complex augments or otherwise facilitates the ubiquitination of a protein. In certain aspects of the present invention an SCF complex refers to a complex comprising Skp1 and an F box protein or parts thereof.

In the context of the present invention the term “F-box protein” refers to a protein comprising a characteristic structural motif called the F-box as described in Bai et al, (1996 Cell 86: 263-274) and a protein-protein interaction domain, in particular a WD40 repeat motif or domain. Examples of F-box Proteins include Cdc4 polypeptides, and homologs or portions thereof, preferably portions that interact with a CPD motif (e.g. WD repeat).

A “WD40 repeat”, “WD40 motif”, or “WD repeat domain” is generally defined as a contiguous sequence of about 25 to 50 amino acids with relatively-well conserved sets of amino acids [i.e. Trp-Asp (WD)] at the ends (amino- and carboxyl-terminal) of the sequence. (For reviews see Neer E J, Schmidt C J, Nambudripad R & Smith TF: “The ancient regulatory-protein family of WD-repeat proteins,” Nature 371, 297-300 (1994) PMID: 8090199; and Smith TF, Gaitatzes CG, Saxena K & Neer EJ: “The WD-repeat: a common architecture for diverse functions,” TIBS 24, 181-185 (1999) PMID: 10322433.) A WD repeat motif or domain can also be defined as a domain of an F-box protein that interacts with a CPD motif or like motif.

Examples of WD-repeat-containing proteins are cdc4 polypeptides, Met30 homologues and orthologues (see for example, GenBank Accession No. P39014 or MT30_YEAST—SEQ ID NO.17) and β-TRCP homologues and orthologues (see for example, GenBank Accession No. NP_{—}033901—SEQ ID NO.18). Other WD40 repeat-containing proteins will, however, be appreciated by those skilled in the art. A WD40-repeat protein also includes a part of the protein. A person skilled in the art may conduct searches to identify proteins that contain WD-40 repeats, in particular F-box proteins. For example, on-line databases such as GenBank or SwissProt can be searched, either with an entire sequence of a WD-40-containing protein, or with a consensus WD-40 repeat sequence. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md. The number of WD-40 repeats in a particular protein can range from two to more than eight.

A “Cdc4 Phospho-Degron motif” or “CPD motif” is a motif that targets substrates for ubiquitination by SCF complexes. The motif can be defined by the consensus sequence X²-X³-pThr-Pro-X⁴ (SEQ ID NO.19), more particularly X²-X³-pThr-Pro-X⁴-X⁵-X⁶-X⁷ (SEQ ID NO.20), wherein X² represents Leu, Pro, or Ile, preferably Leu or Ile; X³ represents Leu, Ile, Val, or Pro, preferably Ile, Leu, or Pro; X⁴ represents any amino acid except basic and bulky hydrophobic amino acids, preferably X⁴, X⁵ and X⁶ represent any amino acid except basic and bulky hydrophobic amino acids, preferably X⁴ is any amino acid except Arg, Lys, Tyr, or Trp, more preferably X⁴ is Ile, Val, Pro, or Gln, preferably X⁵ and X⁶ are any amino acid except Arg, Lys, or Tyr and more preferably X⁵ is Gln, Leu, Met, Thr, or Glu, and X⁶ is Gin, Ala, Thr, Glu, or Ser; and X⁷ is any amino acid, preferably not a basic or bulky hydrophobic amino acid, more preferably X⁷ is any amino acid except Arg, Lys, or Tyr, most preferably X⁷ is Leu, Trp, Asp, Pro, or Gly. A CPD motif preferably comprises the consensus sequence -Leu/Gly/Tyr-Pro-pThr-Pro- (SEQ ID NO.21).

A CPD motif containing protein includes proteins comprising the CPD motif including but not limited to Gcn4, Cyclin E, Far1, Ash1, Sic1, Pc17, Cdc16, p27^{kip1}, Cln2, and transcription factors such as β catenin or IKβα, and homologues of these proteins. The term includes but is not limited to all homologs, orthologs, naturally occurring allelic variants, isoforms and precursors of the polypeptides. Other proteins containing CPD motif sequences may be identified with a protein homology search, for example by searching available databases such as GenBank or SwissProt and various search algorithms and/or programs may be used including FASTA, BLAST (available as a part of the GCG sequence analysis package, University of Wisconsin, Madison, Wis.), or ENTREZ (National Center for Biotechnology Information, National Library of Medicine, National Institutes of Health, Bethesda, Md.).

The term “substrate” refers to a protein that interacts with an F-box protein targeting it for ubiquitin-dependent proteolysis, or a protein targeted for F-box dependent degradation. Examples of substrates are CPD motif containing proteins including Gcn4, CyclinE, Far1, Ash1, Sic1, Pc17, Cdc16, p27^{kip1}; Cln2, and, transcription factors such as β catenin or Iκβα. The term also refers to a part of a protein that interacts with an F-box protein, including a CPD motif, and analogues of substrates or parts thereof

A “ligand” refers to a compound or entity that associates with a binding pocket, or modulators of an F-box protein or SCF E3 ubiquitin ligase, including inhibitors. A ligand may be designed rationally by using a model according to the present invention.

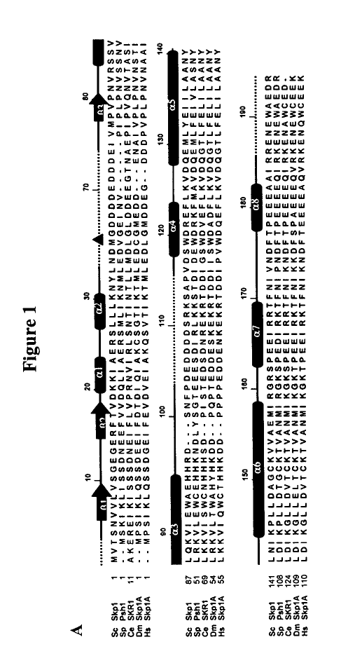

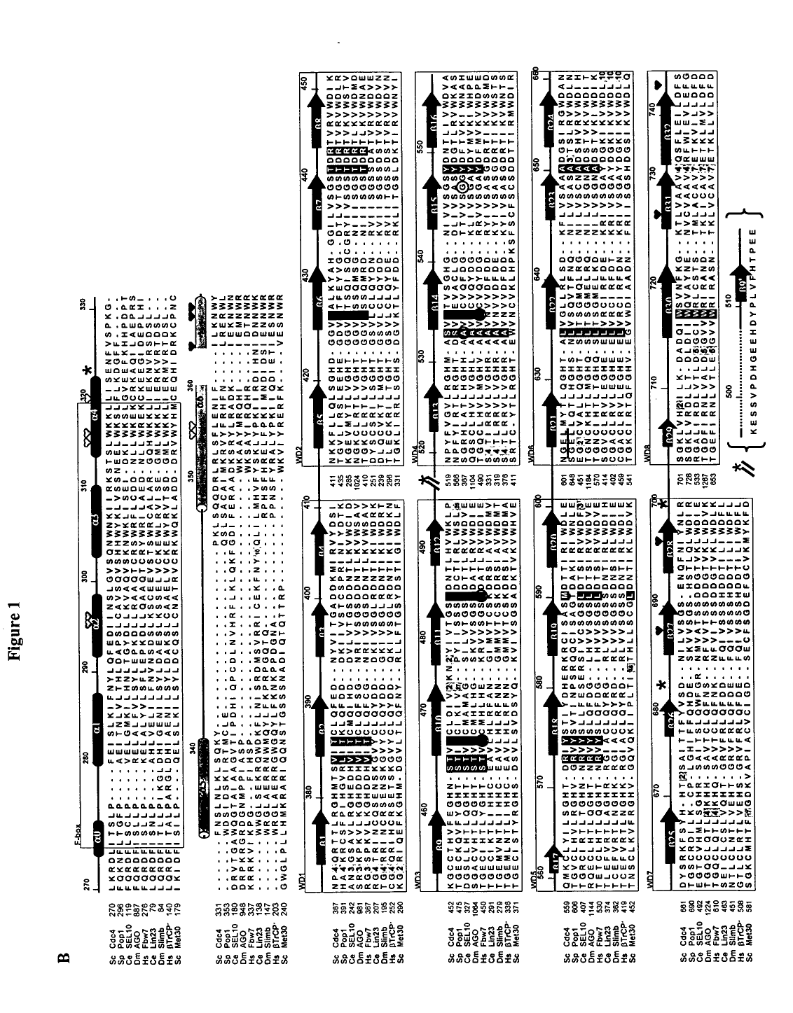

The terms “cdc4 polypeptide” is used to refer to polypeptides of the cdc4 family of proteins characterized by an F-box motif and WD repeats. The term includes but is not limited to all homologs, orthologs, naturally occurring allelic variants, isoforms and precursors of the polypeptides of GenBank Accession Nos. S56245 or SEQ ID NO. 22 (Saccharomyces cerevisiae cdc4), CAA65538 or SEQ ID NO. 23 (Candida albicans cdc4), AAL07271 or SEQ ID NO.24 (human cdc4), AAC47809 or SEQ ID NO.25 (sel-10), AAK57547 or SEQ ID NO.26 (Homo sapiens F-box protein FBW7), and AAG09623F or SEQ ID NO. 27 (Homo sapiens F box protein FBX30). In general, for example, naturally occurring allelic variants will share significant homology (70-90%) to these sequences. Allelic variants may contain conservative amino acid substitutions from cdc4 sequences or will contain a substitution of an amino acid from a corresponding position in a cdc4 homologue such as, for example, the human homologue. [See Strohmaier,H., Nature 413 (6853), 316-322 (2001) for a description and sequence of human cdc4]. The term also includes the mutant cdc4 polypeptides described herein. FIG. 1 shows a structure based sequence alignment of cdc4 orthologs and paralogs.

The term “cdc53” or “cdc53 polypeptide” is used interchangeably herein with the term “cullins” when referring to a vertebrate homolog of the yeast cdc53 protein. The term “cullin polypeptide” or “cullin protein”, refers to a member of the cullins family, e.g., any one of cul-1, -2, -3, -4, -5, or -6. The term includes but is not limited to all homologs, naturally occurring allelic variants, isoforms and precursors of a cdc53 polypeptide or cullin of GenBank Accession Nos. AAB38821 or SEQ ID NO. 28 (Saccharomyces cerevisiae cdc53), AAC36304 or SEQ ID NO. 29 (Homo sapiens cullin 3), AAC51190 or SEQ ID NO. 30 (Homo sapiens cullin 2), NP 003581 or SEQ ID NO. 31 (Homo sapiens cullin 3), AF126404_{—}1 or SEQ ID NO. 32 (Homo sapiens cullin 2), CUL1_CAEEL or SEQ ID NO. 33 (Caenorhabditis elegans cullin 1), AAA85085 or SEQ ID NO. 34 (Drosophila melanogaster cullin 1) and the cullins described in Kipreos ET (Cell 1996 Jun. 14;85(6):829-39). In general for example, naturally occurring allelic variants of cdc53 will share significant homology (70-90%) to the cdc53 or cullin sequences. Allelic variants may contain conservative amino acid substitutions from the cdc4 sequence or will contain a substitution of an amino acid from a corresponding position in a cdc4 homolog such as, for example, the human homolog.

The term “Skp1” or “Skp1 polypeptide” is used to refer to polypeptides that connect cell cycle regulators to the ubiquitin proteolysis machinery by associating with F-box proteins through the F-box motif. The term includes but is not limited to all homologs, naturally occurring allelic variants, isoforms and precursors of Skp1 of GenBank Accession Nos. SKP1_SCHPO or SEQ ID NO. 35 (Schizosaccharomyces pombe), BAB62325 or SEQ ID NO. 36 (Schizosaccharomyces pombe), AAC49492 or SEQ ID NO. 37 (Saccharomyces cerevisiae), and AAB17500 or SEQ ID NO. 38 (Saccharomyces cerevisiae). In general, for example, naturally occurring allelic variants of Skp1 will share significant homology (70-90%) to the Skp1 sequences. Allelic variants may contain conservative amino acid substitutions from the Skp1 sequence or will contain a substitution of an amino acid from a corresponding position in a Skp1 homolog such as, for example, the human homolog. FIG. 1 shows a structure based sequence alignment of Skp1 homologues.

A CPD motif and WD repeat or proteins containing same, cdc4 polypeptides, cdc53, Skp1, substrates, and SCF complexes, may be from any species, particularly a mammalian species, including bovine, ovine, porcine, murine, equine, preferably the human species, and from any source, whether natural, synthetic, semi-synthetic, or recombinant.

The term “agonist” of a binding pocket refers to a compound or ligand that interacts with the binding pocket and maintains or increases the activity of the binding pocket to which it binds. The term includes partial agonists and inverse agonists. Agonists may include proteins, peptides, nucleic acids, carbohydrates, or any other molecules that bind to a binding pocket. Agonists also include a molecule derived from a binding pocket. Peptide mimetics, synthetic molecules with physical structures designed to mimic structural features of particular peptides, may serve as agonists. The stimulation may be direct, or indirect, or by a competitive or non-competitive mechanism. The term includes partial agonists and inverse agonists.

As used herein, the term “partial agonist” means an agonist that is unable to evoke the maximal response of a biological system, even at a concentration sufficient to saturate the specific receptors.

As used herein, the term “partial inverse agonist” is an inverse agonist that evokes a submaximal response to a biological system, even at a concentration sufficient to saturate the specific receptors. At high concentrations, it will diminish the actions of a full inverse agonist.

The term “antagonist”, as used herein, refers to a ligand or compound that binds a binding pocket but does not maintain the activity of the binding pocket to which it binds. The term can also includes a ligand that reduces the action of another agent, such as an agonist. An antagonistic action may result from a combination of the substance being antagonised (chemical antagonism) or the production of an opposite effect through a different protein (functional antagonism or physiological antagonism) or as a consequence of competition for the binding site of an intermediate that links a protein to the effect observed (indirect antagonism). The antagonist may act at the same site as the agonist (competitive antagonism). Antagonists may include proteins, peptides, nucleic acids, carbohydrates, or any other molecules that bind to a binding pocket. Antagonists also include a molecule derived from a binding pocket. Peptide mimetics, synthetic molecules with physical structures designed to mimic structural features of particular peptides, may serve

As used herein, the term “competitive antagonism” refers to the competition between an agonist and an antagonist for a binding pocket of a protein that occurs when the binding of agonist and antagonist becomes mutually exclusive. This may be because the agonist and antagonist compete for the same binding sites or pockets, or combine with adjacent but overlapping sites. A third possibility is that different sites are involved but that they influence the receptor macromolecules in such a way that agonist and antagonist molecules cannot be bound at the same time. If the agonist and antagonist form only short lived combinations with a binding pocket so that equilibrium between agonist, antagonist and binding pocket is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare receptors remain.

By being “derived from” a binding pocket is meant any molecular entity which is identical or substantially equivalent to the binding pocket. A peptide derived from a binding pocket may encompass the amino acid sequence of a naturally occurring binding pocket, any portion of that binding pocket or other molecular entity that functions to bind to an associated or interacting binding pocket. A peptide derived from such a binding pocket will interact directly or indirectly with an associated molecule in such a way as to mimic the native binding pocket. Such peptides may include competitive inhibitors, peptide mimetics, and the like. The entity will not include a full length sequence of a wild-type molecule. Peptide mimetics, synthetic molecules with physical structures designed to mimic structural features of particular peptides, may serve as inhibitors or enhancers.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or agonist or antagonist (i.e. enhancer or inhibitor) of a binding pocket. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a motif, peptide, or agonist or antagonist (i.e. enhancer or inhibitor) of the invention.

Sequences are “homologous” or considered “homologs” when at least about 70% (preferably at least about 80 to 90%, and most preferably at least 95%) of the nucleotides or amino acids match over a defined length of the molecule. “Substantially homologous” also includes sequences showing identity to the specified sequence. Percent identity can be determined electronically, e.g., by using the MEGALIGN program (DNASTAR, Inc., Madison Wis.) which can create alignments between two or more sequences according to different methods, e.g., the clustal method. (See, e.g., Higgins, D. G. and P. M. Sharp (1988) Gene 73:237-244.) Percent identity can also be determined by other methods known in the art, (e.g., the Jotun Hein method. (See, e.g., Hein, J. (1990) Methods Enzymol. 183:626-645) or by varying hybridization conditions). Preferably, the amino acid or nucleic acid sequences have an alignment score of greater than 5 (in standard deviation units) using the program ALIGN with the mutation gap matrix and a gap penalty of 6 or greater (Dayhoff).

Binding Pocket

“Binding pocket” refers to a region or site of an F-box protein or molecular complex thereof (e.g. Skp1-F-box complex, SCF E3 ubiquitin ligase) involved in substrate selection and/or orientation. As the result of its shape, a binding pocket associates with another region of an F-box protein or SCF complex or with a substrate or a part thereof.

In an aspect of the invention a binding pocket comprises one or more of the residues involved in selection and/or orientation of a substrate or ligand.

In an aspect of the invention a binding pocket is provided that comprises the WD40 repeat domain of an F-box protein. In another embodiment the binding pocket comprises a WD40 repeat domain and a helical linker of an F-box protein. In a further embodiment, the binding pocket comprises a WD40 repeat domain, a helical linker and an F-box domain of an F-box protein. In an embodiment the F-box protein is a cdc4 polypeptide or portion thereof.

A binding pocket of the invention may comprise a WD40 repeat domain characterized by one or more of the following characteristics:

(a) a 7 or 8 blade β-propeller structure, in particular a 8 blade β-propeller structure;

(b) a disk like structure characterized by a cavity in the middle and two opposing circular surfaces of different size;

(c) a conical frustum of about 40 Å top surface and about 50 Å bottom surface, an overall thickness of 30 Å and a central pore of 6 Å diameter; and

(d) a CPD binding site on the top surface of the frustum of (c) and running across the edge of the pore, while the bottom surface of the frustum links to the F-box domain.

A binding pocket of the invention may be characterized by one or more of the following characteristics:

(i) a dedicated pThr-Pro binding pocket;

(ii) a deep hydrophobic pocket that selects hydrophobic residues N-terminal to the phosphorylation site of a CPD, and

(iii) a through space electrostatic selection against basic residues C-terminal to the phosphorylation site of a CPD.

A binding pocket of the invention may comprise a helical linker characterized by a helices that form a stalk and pedestal like structure that connects and orients a WD repeat domain. The helical linker binding pocket can also be characterized by one or more of the following:

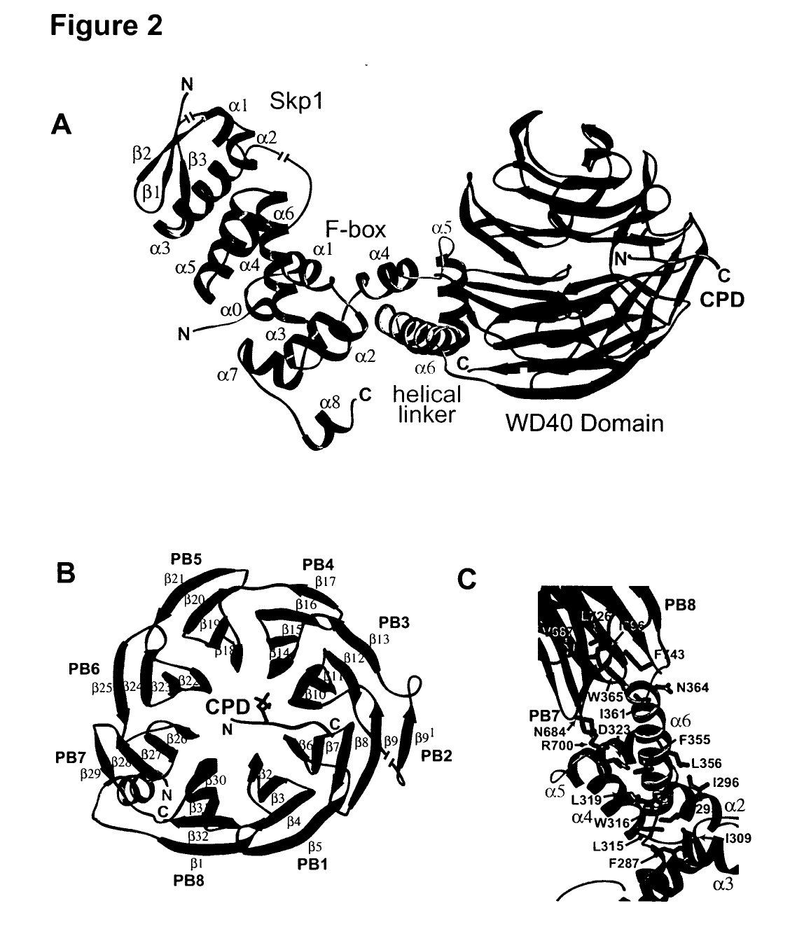

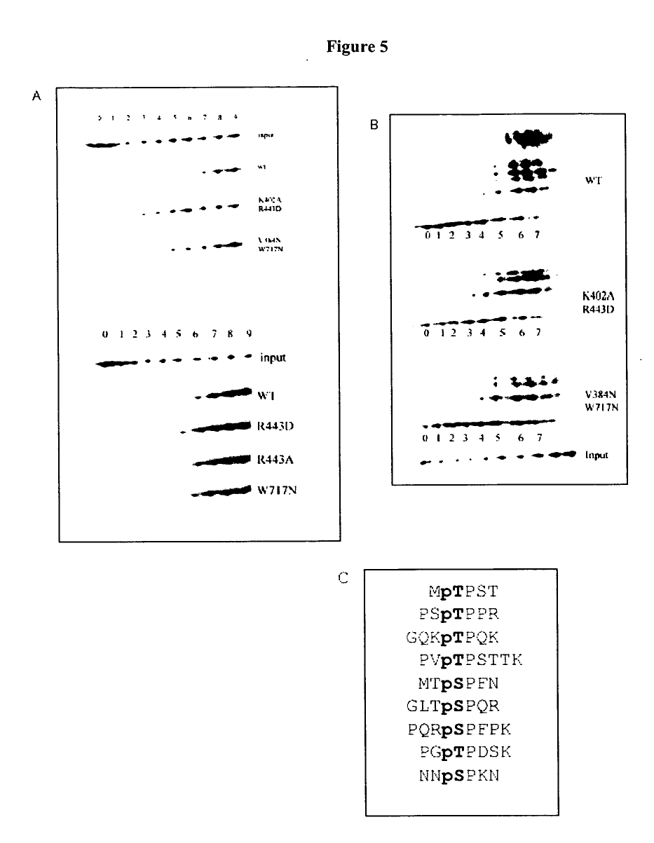

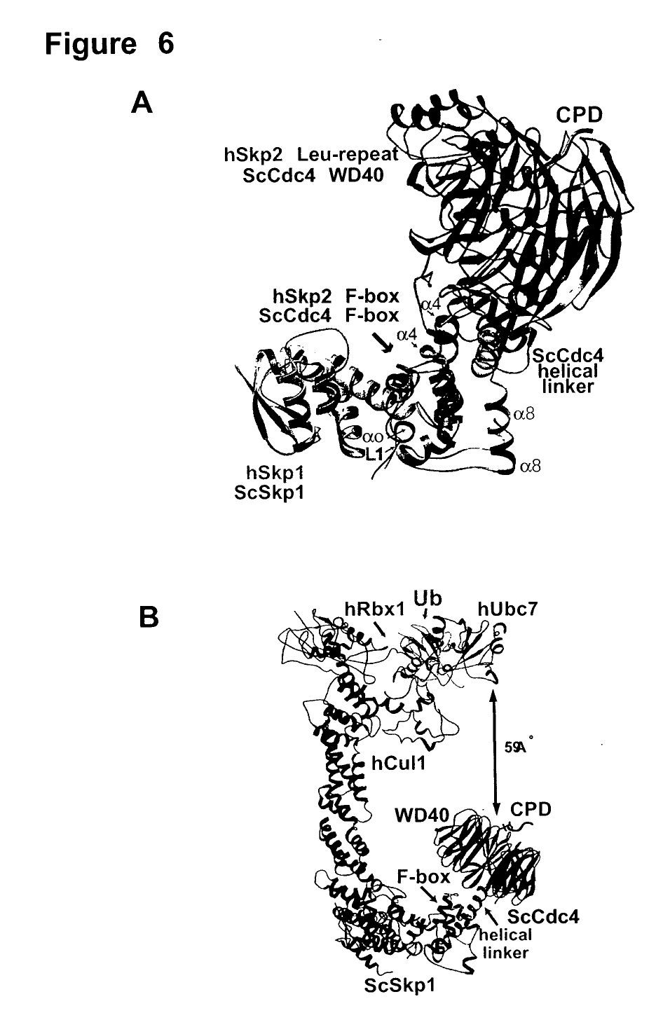

(a) a helix (e.g. α6 in FIG. 2 or FIG. 6) that is 30 Å in length and is anchored at its N-terminus to the hydrophobic core of the F-box/helical extension and at its C-terminus to the hydrophobic core of a WD repeat domain,

(b) the helix of (a) (e.g. α6) anchored at its amino terminus to an F-box through hydrophobic interactions (e.g. involving α6 residues Phe 355, Leu356, and F box residues Ile295, Ile296, Leu315, Leu 319 and Trp316 of Cdc4 or the corresponding residues in Cdc4 homologs, variants, precursors etc.);

(c) a second helix (e.g. helix 5) packed along the base of the helix of (a) or (b) opposite to the F-box domain through hydrophobic interactions (e.g. involving Tyr342, Leu338, and Leu 334 of Cdc4 or the corresponding residues in Cdc4 homologs, variants, precursors etc.);

(d) the helix of (a) (e.g. helix α6) anchored at its C-terminus through hydrophobic interactions;

(e) a C-terminal end of helix α6 inserted obliquely between propeller blades P7 and β8 of a WD40 domain through van der Wals and hydrophobic interactions (e.g. involving Trp365 and Ile361 with WD40 domain residues Val687, Ile696, Leu726, and Phe743 in β-propeller blades 7 and 8 of Cdc4 or the corresponding residues in Cdc4 homologs, variants, precursors etc.).

A CPD motif binding pocket of the invention may comprise a hydrophobic pocket that surrounds the open central channel of a 7 or 8 blade WD repeat propeller. A binding pocket of Cdc4 is more particularly characterized by one or more of the following:

(a) a WD repeat domain surface composed of invariant and highly conserved residues from β-propeller blades;

(b) a three-sided pocket formed by Trp426, Thr386, and Arg 485 (or the corresponding residues in Cdc4 homologs, variants, precursors etc.);

(c) a three-sided pocket formed by Trp426, Thr441, Thr 465, and Arg 485 (or the corresponding residues in Cdc4 homologs, variants, precursors etc.);

(d) a hydrophobic pocket composed of Trp 426, Trp 717, Thr 386, and Val 384 (or the corresponding residues in Cdc4 homologs, variants, precursors etc.);

(e) a pocket formed by Leu634, Met590, and Tyr574 (or the corresponding residues in Cdc4 homologs, variants, precursors etc.); and

(f) a pocket formed by Arg485, Arg467, Arg534, Tyr548, and Arg572 (or the corresponding residues in Cdc4 homologs, variants, precursors etc.);

A binding pocket may comprise one or more of the amino acid residues for an F-box protein crystal or F-box protein—substrate crystal identified in Table 3 or Table 4. In an aspect the binding pocket comprises the atomic contacts of atomic interactions 1 to 4 or interactions 5 to 8/9 identified in Table 3 or Table 4. In an aspect of the invention the binding pocket comprises all of the amino acid residues identified in Table 3 or Table 4.

The term “binding pocket” (BP) also includes a homolog of the binding pocket or a portion thereof. As used herein, the term “homolog” in reference to a binding pocket refers to a binding pocket or a portion thereof which may have deletions, insertions or substitutions of amino acid residues as long as the binding specificity is retained. In this regard, deliberate amino acid substitutions may be made on the basis of similarity in polarity, charge, solubility, hydrophobicity, hydrophilicity, and/or the amphipathic nature of the residues as long as the binding specificity of the binding pocket is retained.

As used herein, the term “portion thereof” means the structural coordinates corresponding to a sufficient number of amino acid residues of a binding pocket (or homologs thereof) that are capable of associating with a substrate (e.g. CPD motif) or ligand. For example, the structural coordinates provided in a crystal structure may contain a subset of the amino acid residues in a binding pocket which may be useful in the modelling and design of compounds that bind to the binding pocket.

Crystal

The invention provides crystal structures. As used herein, the term “crystal” or “crystalline” means a structure (such as a three dimensional (3D) solid aggregate) in which the plane faces intersect at definite angles and in which there is a regular structure (such as internal structure) of the constituent chemical species. The term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer.

In one aspect, the crystal is usable in X-ray crystallography techniques. Here, the crystals used can withstand exposure to X-ray beams used to produce a diffraction pattern data necessary to solve the X-ray crystallographic structure. A crystal may be characterized as being capable of diffracting x-rays in a pattern defined by one of the crystal forms depicted in Blundel et al 1976, Protein Crystallography, Academic Press.

A crystal of the invention is generally produced in a laboratory; that is, it is an isolated crystal produced by an individual.

The invention contemplates a crystal comprising a binding pocket of the invention, in particular a binding pocket of an F-box protein or SCF complex or portion thereof, involved in substrate selection and/or orientation.

In an aspect of the invention a crystal is provided that comprises the WD40 repeat domain of an F-box protein, in particular Cdc4. In another embodiment the crystal comprises a WD40 repeat domain and a helical linker of an F-box protein. In a further embodiment, the crystal comprises a WD40 repeat domain, a helical linker and an F-box domain of an F-box protein. In an embodiment the F-box protein is a cdc4 polypeptide or portion thereof.

A crystal of the invention comprising a WD40 repeat domain, in particular a Cdc4 polypeptide WD40 repeat domain, may be characterized by one or more of the following characteristics:

(a) a 7 or 8 blade β-propeller structure, in particular a 8 blade β-propeller structure;

(b) a disk like structure characterized by a cavity in the middle and two opposing circular surfaces of different size;

(c) a conical frustum of about 40 Å top surface and about 50 Å bottom surface, an overall thickness of 30 Å and a central pore of 6 Å diameter; and

(d) a CPD binding site on the top surface of the frustum of (c) and running across the edge of the pore, while the bottom surface of the frustum links to the F-box domain.

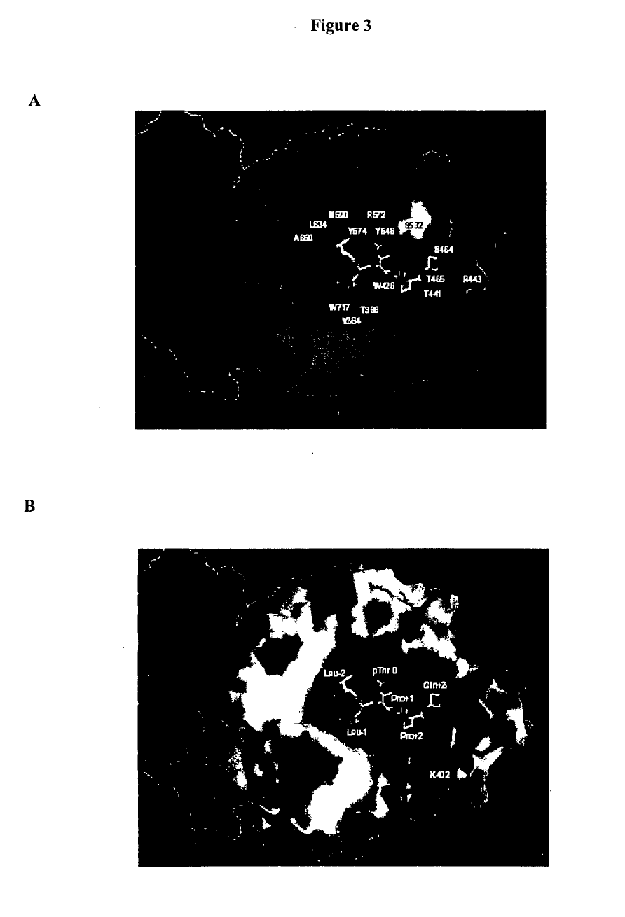

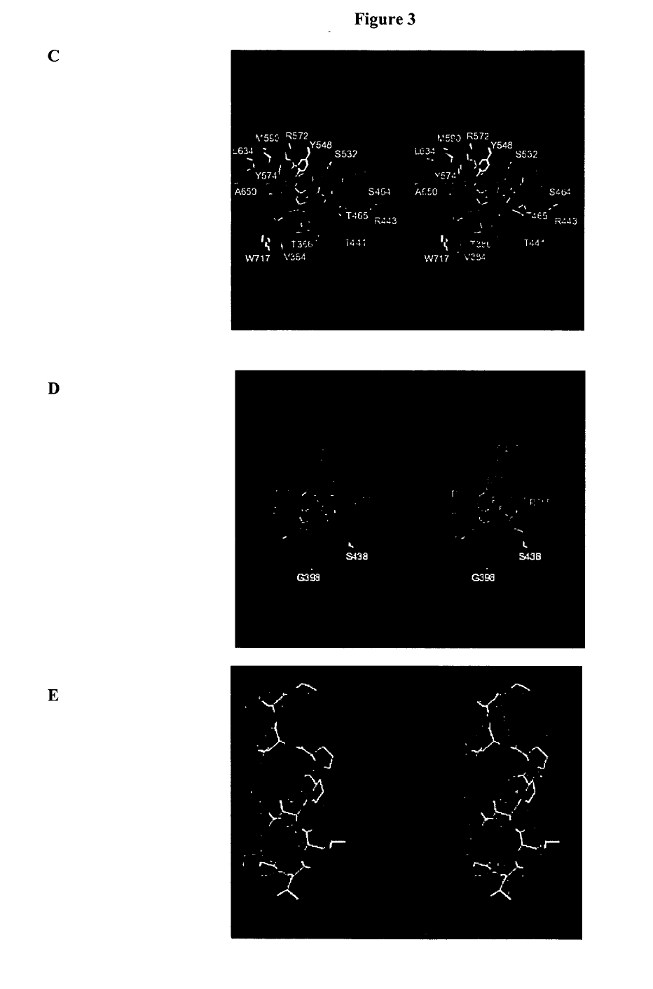

Each blade of the α-propeller structure can be further characterized by 4 anti-parallel α-strands. The disk like structure can also be characterized by a smaller surface comprising a CPD binding site and a bottom surface anchored by a helix (e.g. helix α6) of a helical extension of the F-box protein. As illustrated in FIGS. 2 and 3 the structure is further characterized by β-propeller blade 2 consisting of 5β-strands and a strand β9′ forming a parallel arrangement with strand β9.

A crystal of a binding pocket of an F-box protein of the invention, in particular a Cdc4 polypeptide, may be characterized by one or more of the following characteristics:

(i) a dedicated pThr-Pro binding pocket;

(ii) a deep hydrophobic pocket that selects hydrophobic residues N-terminal to the phosphorylation site of a CPD motif, and

(iii) a through space electrostatic selection against basic residues C-terminal to the phosphorylation site of a CPD motif.

In a preferred embodiment, a crystal of a WD40 repeat domain has the structure illustrated in FIG. 2 or 3.

A crystal of the invention can comprise a helical linker characterized by α helices that form a stalk and pedestal like structure that connects and orients a WD repeat domain. A helical linker structure of a Cdc4 polypeptide can also be characterized by one or more of the following structures: (a) a helix (e.g. α6 in FIG. 2 or FIG. 6) that is

30 Å in length and is anchored at its N-terminus to the hydrophobic core of the F-box/helical extension and at its C-terminus to the hydrophobic core of a WD repeat domain,

(b) the helix of (a) (e.g. α6) anchored at its amino terminus to an F-box through hydrophobic interactions (e.g. involving α6 residues Phe 355, Leu356, and F box residues Ile295, Ile296, Leu315, and Trp316 or the corresponding residues in Cdc4 homologs, variants, precursors etc.));

(c) a second helix (e.g. helix 5) packed along the base of the helix of (a) or (b) opposite to the F-box domain through hydrophobic interactions (e.g. involving Tyr342, Leu338, and Leu 334) (or the corresponding residues in Cdc4 homologs, variants, precursors etc.);

(d) the helix of (a) (e.g. helix α6) anchored at its C-terminus through hydrophobic interactions;

(e) a C-terminal end of helix α6 inserted obliquely between propeller blades β7 and β8 of the WD40 domain through van der Wals and hydrophobic interactions (e.g. involving Trp365 and Ile361 with WD40 domain residues Val687, 11e696, Leu726, and Phe743 in β-propeller bnlades 7 and 8 (or the corresponding residues in Cdc4 homologs, variants, precursors etc.).

In a preferred embodiment, a crystal of a helical linker has the structure illustrated in FIG. 2.

A crystal of the invention may comprise a CPD motif binding pocket that is characterized by a hydrophobic pocket that surrounds the open central channel of a 7 or 8 blade WD repeat propeller. A crystal of a Cdc4 polypeptide may be more particularly characterized by one or more of the following:

(a) a WD repeat domain surface composed of invariant and highly conserved residues from β-propeller blades;

(b) a three-sided pocket formed by Trp426, Thr386, and Arg 485 (or the corresponding residues in Cdc4 homologs, variants, precursors etc.);

(c) a three-sided pocket formed by Trp426, Thr441, Thr 465, and Arg 485 (or the corresponding residues in Cdc4 homologs, variants, precursors etc.);

(d) a hydrophobic pocket composed of Trp 426, Trp 717, Thr 386, and Val 384(or the corresponding residues in Cdc4 homologs, variants, precursors etc.);

(e) a pocket formed by Leu634, MetS90, and Tyr574 (or the corresponding residues in Cdc4 homologs, variants, precursors etc.); and

(f) a pocket formed by Arg485, Arg467, Arg534, Tyr548, and Arg572 (or the corresponding residues in Cdc4 homologs, variants, precursors etc.).

In a preferred embodiment, a crystal of a CPD motif binding pocket has the structure illustrated in FIG. 3, 4, 6 or 7

In a further aspect of the invention a crystal is provided comprising an F-box domain comprising five a helices. In a preferred embodiment, a crystal of an F-box domain has the structure illustrated in FIG. 2 or FIG. 6.

A crystal of the invention may comprise an F-box protein characterized by one or more of the following:

(a) an F-box domain consisting of five a helices;

(b) a WD 40 repeat domain characterized by 7 or 8 copies of a WD40 repeat motif forming a 7 or 8 blade α-propeller structure; and

(c) two a helices that together with two a helices of the F-box domain forming a stalk and pedestal like structure that connects and orients the WD40 domain.

With reference to a crystal of the present invention, residues in a binding pocket may be defined by their spatial proximity to a substrate or ligand in the crystal structure. For example, a binding pocket may be defined by its proximity to a substrate molecule, or modulator.

A crystal of the invention includes a binding pocket in association with one or more moieties, including heavy-metal atoms i.e. a derivative crystal, or one or more substrates or ligands i.e. a co-crystal.

The term “associate”, “association” or “associating” refers to a condition of proximity between a moiety (i.e. chemical entity or compound or portions or fragments thereof), and a binding pocket. The association may be non-covalent i.e. where the juxtaposition is energetically favored by for example, hydrogen-bonding, van der Waals, or electrostatic or hydrophobic interactions, or it may be covalent.

The term “heavy-metal atoms” refers to an atom that can be used to solve an x-ray crystallography phase problem, including but not limited to a transition element, a lanthanide metal, or an actinide metal. Lanthanide metals include elements with atomic numbers between 57 and 71, inclusive. Actinide metals include elements with atomic numbers between 89 and 103, inclusive.

Multiwavelength anomalous diffraction (MAD) phasing may be used to solve protein structures using selenomethionyl (SeMet) proteins. Therefore, a complex of the invention may comprise a crystalline binding pocket with selenium on the methionine residues of the protein.

A crystal may comprise a complex between a binding pocket and one or more substrates or ligands. In other words the binding pocket may be associated with one or more ligands or molecules in the crystal. The ligand may be any compound that is capable of stably and specifically associating with the binding pocket. A ligand may, for example, be a modulator or analogue thereof. Therefore, a crystal may comprise a binding pocket comprising two or more of the amino acid residues of an F-box protein structure as described herein, that are capable of associating with or coordinating a CPD motif as described herein.

In an embodiment, a crystal of the invention comprises a complex between a binding pocket, and a substrate or analogue thereof. Therefore, the present invention also provides a crystal comprising a binding pocket of an F-box protein or a SCF complex and a substrate or analogue thereof. A substrate may be for example, a CPD motif or CPD motif containing protein. An analog of a substrate is one which mimics the substrate molecule, binding in the binding pocket, but which is incapable (or has a significantly reduced capacity) to take part in SCF E3 ubiquitin ligase activity.

In an embodiment, a crystal comprising a WD repeat domain of a Cdc4 polypeptide and a CPD motif is provided, which is characterized by one or more of the following:

(a) a WD 40 repeat domain characterized by 7 or 8 copies of a WD40 repeat motif forming a 7 or 8 blade β-propeller structure comprising β-propeller blades 1, 2, 3, 4, 5, 6, and 7, and optionally 8;

(b) the CPD motif binds in an extended manner across β-propeller blade 2 with the N-terminus oriented toward the central cavity of the WD repeat domain and the C-terminus oriented towards the outer rim;

(c) the CPD binding surface of the WD repeat domain is composed of invariant and highly conserved residues from β-propeller blades 1 to 6 and optionally 8;

(d) a P0 phosphate pThr of the CPD motif forms direct electrostatic interactions with the guanidium groups of Arg 485, Arg 467, and Arg 534 and a direct hydrogen bond with the side chain of Tyr 548 (or the corresponding residues in Cdc4 homologs, variants, precursors etc.);

(e) P+1 proline side chains of the CPD motif project into a three-sided pocket on the CPD binding surface formed by the side chain of Trp 426 and Arg485 or Trp 426, Thr441, Thr465, and Arg 485 (or the corresponding residues in Cdc4 homologs, variants, precursors etc.); and

(f) P+1 leucine side chain of the CPD motif is oriented towards a hydrophobic pocket composed of residues Trp 426, Trp 717, Thr 386, and Val 384 (or the corresponding residues in Cdc4 homologs, variants, precursors etc.).

In a preferred embodiment, a crystal of a complex of a WD repeat domain and a CPD motif has the structure illustrated in FIG. 2, 3, 4, 6, or 7.

A crystal or secondary or three-dimensional structure of a binding pocket of an F-box protein, may be specifically defined by one or more of the atomic contacts of the atomic interactions identified in Table 3 or Table 4. The atomic interactions in Table 3 or Table 4 are defined therein by an atomic contact (more preferably, a specific atom of an amino acid residue where indicated) on the F box protein, in particular on the WD40 repeat domain or helical linker, and an atomic contact (more preferably, a specific atom of an amino acid residue where indicated) on a substrate e.g. CPD motif, or an atomic contact (more preferably, a specific atom of an amino acid residue where indicated) on another region of the F-box protein (e.g. helical linker or F-box domain). In certain embodiments, a crystal of the invention comprises the atomic contacts of atomic interactions 1 to 8 identified in Table 3 or Table 4. In certain particular embodiments a crystal is provided comprising the atomic contacts of atomic interactions 1 to −4 or 5 to 8. Preferably, a crystal is defined by the atoms of the atomic contacts in the binding pocket having the structural coordinates for the atoms listed in Table 6.

A structure of a complex may be defined by selected intermolecular contacts, preferably the structural coordinates of the intermolecular contacts as defined in Table 6, preferably interactions 5 to 8.

A crystal of the invention may comprise one or more of the following groups of amino acid residues: (a) Ile 295, Ile 296, Leu 315, Trp 316, Leu 319, Phe 355, and Leu 356; (b) Val 687, Ile 696, Leu 726, Phe 743, Trp 365, and Ile 364; (c) Asn 684, Arg 700, and Glu 323; (d) Arg 485, Arg 467, Arg 534, Tyr 548; (e) Trp 426, Arg 485, Thr 441, and Thr 465; (f) Trp 426, Trp 717, Thr 386, and Val 384; (g) Tyr 574, Thr 386 and Val 384; (h) Tyr 574, Met 590, and Leo 634; and (i) the corresponding residues in Cdc4 homologs, paralogs, variants, or precursors. Preferably the atoms of the amino acid residues have the structural coordinates as set out in Table 6.

A crystal of the invention may enable the determination of structural data for a substrate or ligand. In order to be able to derive structural data for a ligand, or substrate it is necessary for the molecule to have sufficiently strong electron density to enable a model of the molecule to be built using standard techniques. For example, there should be sufficient electron density to allow a model to be built using XTALVIEW (McRee 1992 J. Mol. Graphics. 10 44-46).

A crystal of the invention may belong to space group P3₂. The term “space group” refers to the lattice and symmetry of the crystal. In a space group designation the capital letter indicates the lattice type and the other symbols represent symmetry operations that can be carried out on the contents of the asymmetric unit without changing its appearance.

A crystal of the invention may comprise a unit cell having the following unit dimensions: α=107.7 Å, b=107.7 Å, c=168.3 Å, α=γ=90°, p=120°. The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

In a preferred embodiment, a crystal of the invention has the structural coordinates as shown in Table 6. As used herein, the term “structural coordinates” refers to a set of values that define the position of one or more amino acid residues with reference to a system of axes. The term refers to a data set that defines the three dimensional structure of a molecule or molecules (e.g. Cartesian coordinates, temperature factors, and occupancies). Structural coordinates can be slightly modified and still render nearly identical three dimensional structures. A measure of a unique set of structural coordinates is the root-mean-square deviation of the resulting structure. Structural coordinates that render three dimensional structures (in particular a three dimensional structure of a ligand binding pocket) that deviate from one another by a root-mean-square deviation of less than 5 Å, 4 Å, 3 Å, 2 Å, 1.5 Å, 1.0 Å, or 0.5 Å may be viewed by a person of ordinary skill in the art as very similar.

Variations in structural coordinates may be generated because of mathematical manipulations of the structural coordinates of a structure or binding pocket described herein. For example, the structural coordinates of Table 6 may be manipulated by crystallographic permutations of the structural coordinates, fractionalization of the structural coordinates, integer additions or substractions to sets of the structural coordinates, inversion of the structural coordinates or any combination of the above.

Variations in the crystal structure due to mutations, additions, substitutions, and/or deletions of the amino acids, or other changes in any of the components that make up the crystal may also account for modifications in structural coordinates. If such modifications are within an acceptable standard error as compared to the original structural coordinates, the resulting structure may be the same. Therefore, a ligand that bound to a binding pocket of an F-box protein, would also be expected to bind to another binding pocket whose structural coordinates defined a shape that fell within the acceptable error. Such modified structures of a binding pocket thereof are also within the scope of the invention.

Various computational analyses may be used to determine whether a molecule or the binding pocket thereof is sufficiently similar to all or parts of an F-box or a binding pocket thereof. Such analyses may be carried out using conventional software applications and methods as described herein.

A crystal of the invention may also be specifically characterised by the parameters, diffraction statistics and/or refinement statistics set out in Table 1 or in Table 2.

Illustrations of particular crystals of the invention are shown in FIGS. 2, 3, 4, 6 and 7.

Method of Making a Crystal

The present invention also provides a method of making a crystal according to the invention. The crystal may be formed from an aqueous solution comprising a purified polypeptide comprising an F-box protein including a variant, part, homolog, or fragment thereof (e.g. a binding pocket). A method may utilize a purified polypeptide comprising a binding pocket to form a crystal. A method may utilize one or more purified mutant polypeptides as described herein. In an embodiment, a mutant cdc4 polypeptide is used to make crystals.

The term “purified” in reference to a polypeptide, does not require absolute purity such as a homogenous preparation rather it represents an indication that the polypeptide is relatively purer than in the natural environment. Generally, a purified polypeptide is substantially free of other proteins, lipids, carbohydrates, or other materials with which it is naturally associated, preferably at a functionally significant level for example at least 85% pure, more preferably at least 95% pure, most preferably at least 99% pure. A skilled artisan can purify a polypeptide comprising using standard techniques for protein purification. A substantially pure polypeptide will yield a single major band on a non-reducing polyacrylamide gel. Purity of the polypeptide can also be determined by amino-terminal amino acid sequence analysis.

A polypeptide used in the method may be chemically synthesized in whole or in part using techniques that are well-known in the art. Alternatively, methods are well known to the skilled artisan to construct expression vectors containing a native or mutated protein coding sequence and appropriate transcriptional/translational control signals. These methods include in vitro recombinant DNA techniques, synthetic techniques, and in vivo recombination/genetic recombination. See for example the techniques described in Sambrook et al. (Molecular Cloning: A Laboratory Manual, 2nd Edition, Cold Spring Harbor Laboratory press (1989)), and other laboratory textbooks. (See also Sarker et al, Glycoconjugate J. 7:380, 1990; Sarker et al, Proc. Natl. Acad, Sci. USA 88:234-238, 1991, Sarker et al, Glycoconjugate J. 11: 204-209, 1994; Hull et al, Biochem Biophys Res Commun 176:608, 1991 and Pownall et al, Genomics 12:699-704, 1992).

Crystals may be grown from an aqueous solution containing the purified polypeptide by a variety of conventional processes. These processes include batch, liquid, bridge, dialysis, vapor diffusion, and hanging drop methods. (See for example, McPherson, 1982 John Wiley, New York; McPherson, 1990, Eur. J. Biochem. 189: 1-23; Webber. 1991, Adv. Protein Chem. 41:1-36). Generally, native crystals of the invention are grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

Derivative crystals of the invention can be obtained by soaking native crystals in a solution containing salts of heavy metal atoms. A complex of the invention can be obtained by soaking a native crystal in a solution containing a compound that binds the polypeptide, or they can be obtained by co-crystallizing the polypeptide in the presence of one or more compounds. In order to obtain co-crystals with a compound which binds deep within the tertiary structure of the polypeptide it is necessary to use the second method.

Once the crystal is grown it can be placed in a glass capillary tube and mounted onto a holding device connected to an X-ray generator and an X-ray detection device. Collection of X-ray diffraction patterns are well documented by those skilled in the art (See for example, Ducruix and Geige, 1992, IRL Press, Oxford, England). A beam of X-rays enter the crystal and diffract from the crystal. An X-ray detection device can be utilized to record the diffraction patterns emanating from the crystal. Suitable devices include the Marr 345 imaging plate detector system with an RU200 rotating anode generator.

Multiwavelength anomalous diffraction (MAD) phasing using selenomethionyl (SeMet) proteins may be used to determine a crystal of the invention. Thus, the invention contemplates a method for determining a crystal structure of the invention using a selenomethionyl derivative of an F-box protein or SCF complex, including a variant, part, homolog or fragement thereof.

Methods for obtaining the three dimensional structure of the crystalline form of a molecule or complex are described herein and known to those skilled in the art (see Ducruix and Geige 1992, IRL Press, Oxford, England). Generally, the x-ray crystal structure is given by the diffraction patterns. Each diffraction pattern reflection is characterized as a vector and the data collected at this stage determines the amplitude of each vector. The phases of the vectors may be determined by the isomorphous replacement method where heavy atoms soaked into the crystal are used as reference points in the X-ray analysis (see for example, Otwinowski, 1991, Daresbury, United Kingdom, 80-86). The phases of the vectors may also be determined by molecular replacement (see for example, Naraza, 1994, Proteins 11:281-296). The amplitudes and phases of vectors from the crystalline form determined in accordance with these methods can be used to analyze other related crystalline polypeptides.

The unit cell dimensions and symmetry, and vector amplitude and phase information can be used in a Fourier transform function to calculate the electron density in the unit cell i.e. to generate an experimental electron density map. This may be accomplished using the PHASES package (Furey, 1990). Amino acid sequence structures are fit to the experimental electron density map (i.e. model building) using computer programs (e.g. Jones, TA. et al, Acta Crystallogr A47, 100-119, 1991). This structure can also be used to calculate a theoretical electron density map. The theoretical and experimental electron density maps can be compared and the agreement between the maps can be described by a parameter referred to as R-factor. A high degree of overlap in the maps is represented by a low value R-factor. The R-factor can be minimized by using computer programs that refine the structure to achieve agreement between the theoretical and observed electron density map. For example, the XPLOR program, developed by Brunger (1992, Nature 355:472-475) can be used for model refinement.

A three dimensional structure of the molecule or complex may be described by atoms that fit the theoretical electron density characterized by a minimum R value. Files can be created for the structure that defines each atom by coordinates in three dimensions.

Mutant CDC4 Polypeptides

The present invention provides novel mutant cdc4 polypeptides.

A particular mutant of the present invention is a polypeptide having an amino acid sequence of a cdc4 polypeptide wherein amino acid residues are replaced or deleted providing a cdc4 polypeptide that can be produced by recombinant techniques and retains its activity, for example its ability to associate with a CPD motif.

In an aspect a cdc4 sequence is mutated by deleting the region from the beginning of the F-box domain to the end of the WD40 repeat domain. In particular, terminal residues 1 to 262 and 745 to 779 can be deleted from the cdc4 seqeunce.

Other additions, substitutions, and/or deletions may be made to the cdc4 mutants of the present invention. In an embodiment cdc4 can be engineered to remove flexible loops comprising residues 601 to 604 and 609 to 624.

Particular mutant cdc4 polypeptides of the invention are also identified in Table 5.

The present invention also relates to nucleic acid molecules or polynucleotides encoding a cdc4 mutant polypeptide. The polynucleotides can be used to transform host cells to express the cdc4 mutant polypeptides of the invention. They can also be used as a probe to detect related enzymes.

The present invention still further relates to recombinant vectors that include the nucleic acid molecules of the invention. The nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule. A vector can be used to transform host cells. Therefore, the invention provides host cells containing a vector of the invention. As well, the invention provides methods of making such vectors and host cells.

The mutant cdc4 polypeptides of the invention can be encoded, expressed, and purified by any one of a number of methods known to those skilled in the art. Preferred production methods will depend on many factors including the costs and availability of materials and other economic considerations. The optimum production procedure for a given situation will be apparent to those skilled in the art through minimal experimentation.

In accordance with an aspect of the present invention, there is provided a process for producing a cdc4 mutant polypeptide by recombinant techniques utilizing the nucleic acid molecules of the invention. The method may comprise culturing recombinant host cells containing a nucleic acid sequence encoding a cdc4 mutant polypeptide, under conditions promoting expression of the cdc4 mutant polypeptide, and subsequent recovery of the cdc4 mutant polypeptide.

The invention further broadly contemplates a recombinant cdc4 mutant polypeptide obtained using a method of the invention.

A cdc4 mutant polypeptide of the invention may be conjugated with other molecules, such as polypeptides, to prepare fusion polypeptides or chimeric polypeptides. This may be accomplished, for example, by the synthesis of N-terminal or C-terminal fusion polypeptides.

The invention further contemplates antibodies having specificity against a cdc4 mutant polypeptide of the invention. Antibodies may be labeled with a detectable substance and used to detect cdc4 mutant polypeptides. In another embodiment, the invention provides an isolated antibody that binds specifically to a cdc4 mutant polypeptide.

The cdc4 mutant polypeptides of the present invention are particularly well suited for use in screening methods for identifying modulators of cdc4 or SCF complexes.

Still further the invention provides a method for evaluating a test compound for its ability to modulate the biological activity of a cdc4 polypepide. In this application, “modulate” refers to a change or an alteration in the biological activity of a cdc4 polypeptide. Modulation may be an increase or a decrease in activity, a change in characteristics (e.g. kinetic characteristics), or any other change in the biological, functional, or immunological properties of the polypeptide.

The substances and compounds identified using the methods of the invention, may be used to modulate the biological activity of a cdc4 polypeptide or a SCF complex, and they may be used in the treatment of conditions mediated by a cdc4 polypeptide or SCF complex. Accordingly, the substances and compounds may be formulated into compositions for administration to individuals suffering from one or more of these conditions. Therefore, the present invention also relates to a composition comprising one or more of a substance or compound identified using a method of the invention, and a pharmaceutically acceptable carrier, excipient or diluent. A method for treating or preventing these conditions is also provided comprising administering to a patient in need thereof, a composition of the invention.

Model

A crystal structure of the present invention may be used to make a model of a binding pocket of a SCF E3 ubiquitin ligase, in particular an F-box protein, that is involved in substrate selection and/or orientation. A model may, for example, be a structural model or a computer model. A model may represent the secondary, tertiary and/or quaternary structure of the binding pocket. The model itself may be in two or three dimensions. It is possible for a computer model to be in three dimensions despite the constraints imposed by a conventional computer screen, if it is possible to scroll along at least a pair of axes, causing “rotation” of the image.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

Preferably, modelling is performed using a computer and may be further optimized using known methods. This is called modelling optimisation.

An integral step to an approach of the invention for designing modulators (e.g. inhibitors) of a subject F-box protein or SCF complex involves construction of computer graphics models of a binding pocket of the invention which can be used to design pharmacophores by rational drug design. For instance, for an inhibitor to interact optimally with the subject binding pocket, it will generally be desirable that it have a shape which is at least partly complimentary to that of a particular binding pocket of the protein, as for example those binding pockets of the protein which are involved in recognition of a ligand (e.g. substrate). Additionally, other factors, including electrostatic interactions, hydrogen bonding, hydrophobic interactions, desolvation effects, and cooperative motions of ligand and receptor, all influence the binding effect and should be taken into account in attempts to design bioactive modulators (e.g. inhibitors).

As described herein, a computer-generated molecular model of the subject binding pockets can be created. In preferred embodiments, at least the Cα-carbon positions of the binding pockets are mapped to a particular coordinate pattern, such as the coordinates for a binding pocket in Table 6, by homology modeling, and the structure of the protein and velocities of each atom are calculated at a simulation temperature (Tₒ) at which the docking simulation is to be determined. Typically, such a protocol involves primarily the prediction of side-chain conformations in the modeled binding pocket, while assuming a main-chain trace taken from a tertiary structure such as provided in Table 6 and the Figures. Computer programs for performing energy minimization routines are commonly used to generate molecular models. For example, both the CHARMM (Brooks et al. (1983) J Comput Chem 4:187-217) and AMBER (Weiner et al (1981) J. Comput. Chem. 106: 765) algorithms handle all of the molecular system setup, force field calculation, and analysis (see also, Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488). At the heart of these programs is a set of subroutines that, given the position of every atom in the model, calculate the total potential energy of the system and the force on each atom. These programs may utilize a starting set of atomic coordinates, such as the coordinates provided in Table 6, the parameters for the various terms of the potential energy function, and a description of the molecular topology (the covalent structure). Common features of such molecular modeling methods include: provisions for handling hydrogen bonds and other constraint forces; the use of periodic boundary conditions; and provisions for occasionally adjusting positions, velocities, or other parameters in order to maintain or change temperature, pressure, volume, forces of constraint, or other externally controlled conditions.

Most conventional energy minimization methods use the input data described above and the fact that the potential energy function is an explicit, differentiable function of Cartesian coordinates, to calculate the potential energy and its gradient (which gives the force on each atom) for any set of atomic positions. This information can be used to generate a new set of coordinates in an effort to reduce the total potential energy and, by repeating this process over and over, to optimize the molecular structure under a given set of external conditions. These energy minimization methods are routinely applied to molecules similar to the subject proteins as well as nucleic acids, polymers and zeolites.

In general, energy minimization methods can be carried out for a given temperature, Tᵢₛ, which may be different than the docking simulation temperature, Tₒ. Upon energy minimization of the molecule at Tᵢ, coordinates and velocities of all the atoms in the system are computed. Additionally, the normal modes of the system are calculated. It will be appreciated by those skilled in the art that each normal mode is a collective, periodic motion, with all parts of the system moving in phase with each other, and that the motion of the molecule is the superposition of all normal modes. For a given temperature, the mean square amplitude of motion in a particular mode is inversely proportional to the effective force constant for that mode, so that the motion of the molecule will often be dominated by the low frequency vibrations.

After the molecular model has been energy minimized at Tᵢ, the system is “heated” or “cooled” to the simulation temperature, Tᵢ, by carrying out an equilibration run where the velocities of the atoms are scaled in a step-wise manner until the desired temperature, Tₒ, is reached. The system is further equilibrated for a specified period of time until certain properties of the system, such as average kinetic energy, remain constant. The coordinates and velocities of each atom are then obtained from the equilibrated system.

Further energy minimization routines can also be carried out. For example, a second class of methods involves calculating approximate solutions to the constrained EOM for the protein. These methods use an iterative approach to solve for the Lagrange multipliers and, typically, only need a few iterations if the corrections required are small. The most popular method of this type, SHAKE (Ryckaert et al. (1977) J Comput Phys 23:327; and Van Gunsteren et al. (1977) Mol Phys 34:13 11) is easy to implement and scales as O(N) as the number of constraints increases. Therefore, the method is applicable to macromolecules such as F-box proteins. An alternative method, RATTLE (Anderson (1983) J Comput Phys 52:24) is based on the velocity version of the Verlet algorithm. Like SHAKE, RATTLE is an iterative algorithm and can be used to energy minimize the model of the subject protein.

Overlays and super positioning with a three dimensional model of a binding pocket of the invention may be used for modelling optimisation. Additionally alignment and/or modelling can be used as a guide for the placement of mutations on a binding pocket to characterize the nature of the site in the context of a cell.

The three dimensional structure of a new crystal may be modelled using molecular replacement. The term “molecular replacement” refers to a method that involves generating a preliminary model of a molecule or complex whose structural coordinates are unknown, by orienting and positioning a molecule whose structural coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are X-PLOR (Brunger 1992, Nature 355: 472-475), AMoRE (Navaza, 1994, Acta Crystallogr. A50:157-163), the CCP4 package (Collaborative Computational Project, Number 4, “The CCP4 Suite: Programs for Protein Crystallography”, Acta Cryst., Vol. D50, pp. 760-763, 1994), the MERLOT package (P. M. D. Fitzgerald, J. Appl. Cryst., Vol. 21, pp. 273-278, 1988) and XTALVIEW (McCree et al (1992) J. Mol. Graphics 10: 44-46. It is preferable that the resulting structure not exhibit a root-mean-square deviation of more than 3 Å.

Molecular replacement computer programs generally involve the following steps: (1) determining the number of molecules in the unit cell and defining the angles between them (self rotation function); (2) rotating the known structure against diffraction data to define the orientation of the molecules in the unit cell (rotation function); (3) translating the known structure in three dimensions to correctly position the molecules in the unit cell (translation function); (4) determining the phases of the X-ray diffraction data and calculating an R-factor calculated from the reference data set and from the new data wherein an R-factor between 30-50% indicates that the orientations of the atoms in the unit cell have been reasonably determined by the method; and (5) optionally, decreasing the R-factor to about 20% by refining the new electron density map using iterative refinement techniques known to those skilled in the art (refinement).

The quality of the model may be analysed using a program such as PROCHECK or 3D-Profiler [Laskowski et al 1993 J. Appl. Cryst. 26:283-291; Luthy R. et al, Nature 356: 83-85, 1992; and Bowie, J. U. et al, Science 253: 164-170, 1991]. Once any irregularities have been resolved, the entire structure may be further refined.

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structural coordinates of crystal provided by the invention, molecular modelling may be used to determine the structural coordinates of a crystalline mutant or homolog of a SCF complex or F-box binding pocket involved in substrate selection and/or orientation. By the same token a crystal of the invention can be used to provide a model of a substrate or ligand. Modelling techniques can then be used to approximate the three dimensional structure of substrate or ligand derivatives and other components which may be able to mimic the atomic contacts between a substrate or ligand and binding pocket.

Computer Format of Crystals/Models

Information derivable from a crystal of the present invention (for example the structural coordinates) and/or the model of the present invention may be provided in a computer-readable format.

Therefore, the invention provides a computer readable medium or a machine readable storage medium which comprises the structural coordinates of a binding pocket of an SCF complex of F box protein described herein including all or any parts thereof, or substrates or ligands including portions thereof. Such storage medium or storage medium encoded with these data are capable of displaying on a computer screen or similar viewing device, a three-dimensional graphical representation of a molecule or molecular complex which comprises such binding pockets or similarly shaped homologous binding pockets. Thus, the invention also provides computerized representations of the secondary or three-dimensional structures of a binding pocket of the invention, including any electronic, magnetic, or electromagnetic storage forms of the data needed to define the structures such that the data will be computer readable for purposes of display and/or manipulation.

In an aspect the invention provides a computer for producing a three-dimensional representation of a molecule or molecular complex, wherein said molecule or molecular complex comprises a binding pocket defined by structural coordinates of a binding pocket or structural coordinates of atoms of a substrate or ligand, or a three-dimensional representation of a homolog of said molecule or molecular complex, wherein said homolog comprises a binding pocket, or substrate or ligand that has a root mean square deviation from the backbone atoms not more than 1.5 angstroms wherein said computer comprises:

(a) a machine-readable data storage medium comprising a data storage material encoded with machine readable data wherein said data comprises the structural coordinates of a binding pocket or a substrate according to Table 6;

(b) a working memory for storing instructions for processing said machine-readable data;

(c) a central-processing unit coupled to said working memory and to said machine-readable data storage medium for processing said machine readable data into said three-dimensional representation; and

(d) a display coupled to said central-processing unit for displaying said three-dimensional representation.

The invention also provides a computer for determining at least a portion of the structural coordinates corresponding to an X-ray diffraction pattern of a molecule or molecular complex wherein said computer comprises:

(a) a machine-readable data storage medium comprising a data storage material encoded with machine readable data wherein said data comprises the structural coordinates according to Table 6;

(b) a machine-readable data storage medium comprising a data storage material encoded with machine readable data wherein said data comprises an X-ray diffraction pattern of said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structural coordinates; and

(e) a display coupled to said central-processing unit for displaying said structural coordinates of said molecule or molecular complex.

Structural Studies

The present invention also provides a method for determining the secondary and/or tertiary structures of a pol)peptide or part or complexes thereof by using a crystal, or a model according to the present invention. The polypeptide or part thereof may be any polypeptide or part thereof for which the secondary and or tertiary structure is uncharacterised or incompletely characterised. In a preferred embodiment the polypeptide shares (or is predicted to share) some structural or functional homology to a crystal of the present invention. For example, the polypeptide may show a degree of structural homology over some or all parts of the primary amino acid sequence.

The polypeptide may be an F-box protein, or part thereof with a different specificity for a substrate. Alternatively (or in addition) the polypeptide may be an F-box protein from a different species.

The polypeptide may be a mutant of a wild-type F-box protein. A mutant may arise naturally, or may be made artificially (for example using molecular biology techniques). The mutant may also not be “made” at all in the conventional sense, but merely tested theoretically using the model of the present invention. A mutant may or may not be functional.

Thus, using a model of the present invention, the effect of a particular mutation on the overall two and/or three dimensional structure of an F-box protein or SCF complex or the interaction between a binding pocket of an F-box protein or SCF complex and a substrate or ligand can be investigated.

Alternatively, the polypeptide may perform an analogous function or be suspected to show a similar mechanism to an F-box protein.

The polypeptide may also be the same as the polypeptide of the crystal, but in association with a different substrate or ligand (for example, modulator or inhibitor) or cofactor. In this way it is possible to investigate the effect of altering the substrate or ligand with which the polypeptide is associated on the structure of the binding pocket.

Secondary or tertiary structure may be determined by applying the structural coordinates of a crystal or model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a polypeptide sequence based on the structures of known proteins (i.e. an F-box structure or complex thereof described herein). The method utilizes a computer model of a crystal of the present invention (the “known structure”), a computer representation of the amino acid sequence of the polypeptide with an unknown structure, and standard computer representations of the structures of amino acids. The method in particular comprises the steps of; (a) identifying structurally conserved and variable regions in the known structure; (b) aligning the amino acid sequences of the known structure and unknown structure (c) generating co-ordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http://biochem.vt.edu/courses/modeling/homology.htn). Computer programs that can be used in homology modelling are Quanta and the Homology module in the Insight II modelling package distributed by Molecular Simulations Inc, or MODELLER (Rockefeller University, www.iucr.ac.uk/sinris-top/logical/prg-modeller.html).

In step (a) of the homology modelling method, a known structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites. The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442-453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoff et al., Meth. Enzymol. 91: 524-545, 1983), and BLOSUM (Henikoff and Henikoff, Proc. Nat. Acad. Sci. USA 89: 10915-0919, 1992), and the matrices based on alignments derived from three-dimensional structures including that of Johnson and Overington (JO matrices) (J. Mol. Biol. 233: 716-738, 1993).

Alignment based solely on sequence may be used; however, other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating coordinates for the unknown structure, main chain atoms and side chain atoms, both in SCRs and VRs need to be modelled. A variety of approaches known to those skilled in the art may be used to assign co-ordinates to the unknown. In particular, the co-ordinates of the main chain atoms of SCRs will be transferred to the unknown structure. VRs correspond most often to the loops on the surface of the polypeptide and if a loop in the known structure is a good model for the unknown, then the main chain co-ordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent minimizations. Molecular dynamics may also be applied in conjunction with energy minimization.

Molecular replacement involves applying a known structure to solve the X-ray crystallographic data set of a polypeptide of unknown structure. The method can be used to define the phases describing the X-ray diffraction data of a polypeptide of unknown structure when only the amplitudes are known. Thus in an embodiment of the invention, a method is provided for determining three dimensional structures of polypeptides with unknown structure by applying the structural coordinates of a crystal of the present invention to provide an X-ray crystallographic data set for a polypeptide of unknown structure, and (b) determining a low energy conformation of the resulting structure.

The structural coordinates of a crystal of the present invention may be applied to nuclear magnetic resonance (NMR) data to determine the three dimensional structures of polypeptides with uncharacterised or incompletely characterised sturcture. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

Screening Methods

Another aspect of the present invention is the design and identification of agents that inhibit or potentiate an interaction between an F-box protein or an SCF E3 ubiquitin ligase and a substrate. The rationale design and identification of agents can be accomplished by utilizing the structural coordinates that define a binding pocket of the present invention involved in substrate selection and/or orientation.

The structures described herein, and the structures of other polypeptides determined by homology modeling, molecular replacement, and NMR techniques described herein can also be applied to modulator design and identification methods.

The invention contemplates molecular models, in particular three-dimensional molecular models of binding pockets of the present invention involved in substrate selection and/or orientation, and their use as templates for the design of agents able to mimic or inhibit substrate binding (e.g. modulators).

In certain embodiments, the present invention provides a method of screening for a ligand that associates with a binding pocket and/or modulates the function of a F-box protein or SCF complex by using a crystal or a model according to the present invention. The method may involve investigating whether a test compound is capable of associating with or binding a binding pocket, and/or inhibiting or enhancing interactions of atomic contacts in a binding pocket.

In accordance with an aspect of the present invention, a method is provided for screening for a ligand capable of binding to a binding pocket, wherein the method comprises using a crystal or model according to the invention.

In another aspect, the invention relates to a method of screening for a ligand capable of binding to a binding pocket, wherein the binding pocket is defined by the structural coordinates given herein, the method comprising contacting the binding pocket with a test compound and determining if the test compound binds to the binding pocket.

In one embodiment, the present invention provides a method of screening for a test compound capable of interacting with one or more key amino acid residues of a binding pocket of the present invention. For example, a test compound that interacts with one or more of amino acids of a binding pocket may prevent interaction of the F-box protein or complex thereof and its substrate resulting in modification of the SCF E3 ubiquitin ligase activity.

Another aspect of the invention provides a process comprising the steps of:

(a) performing a method of screening for a ligand described above;

(b) identifying one or more ligands capable of binding to a binding pocket; and

(c) preparing a quantity of said one or more ligands.

A further aspect of the invention provides a process comprising the steps of;

(a) performing a method of screening for a ligand as described above;

(b) identifying one or more ligands capable of binding to a binding pocket; and

(c) preparing a pharmaceutical composition comprising said one or more ligands.

Once a test compound capable of interacting with one or more key amino acid residues in a binding pocket of the present invention has been identified, further steps may be carried out either to select and/or modify compounds and/or to modify existing compounds, to modulate the interaction with the key amino acid residues in the binding pocket.

Yet another aspect of the invention provides a process comprising the steps of;

(a) performing the method of screening for a ligand as described above;

(b) identifying one or more ligands capable of binding to a binding pocket;

(c) modifying said one or more ligands capable of binding to a binding pocket;

(d) performing said method of screening for a ligand as described above; and

(e) optionally preparing a pharmaceutical composition comprising said one or more ligands.

In another aspect of the invention, a method of screening for a test compound is provided comprising screening for test compounds that affect (inhibit or potentiate) an interaction between an F-box protein or SCF complex and a substrate as defined by interactions 1 to 4 or 5 to 8/9 in Table 3 or Table 4.

As used herein, the term “test compound” means any compound which is potentially capable of associating with a binding pocket, inhibiting or enhancing interactions of atomic contacts in a binding pocket. If, after testing, it is determined that the test compound does bind to the binding pocket, inhibits or enhances interactions of atomic contacts in a binding pocket, it is known as a “ligand”.

The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptide synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The increasing availability of biomacromolecule structures of potential pharmacophoric molecules that have been solved crystallographically has prompted the development of a variety of direct computational methods for molecular design, in which the steric and electronic properties of substrate binding sites are use to guide the design of potential ligands (Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153). Directed methods generally fall into two categories: (1) design by analogy in which 3-D structures of known molecules (such as from a crystallographic database) are docked to the structure and scored for goodness-of-fit; and (2) de novo design, in which the ligand model is constructed piece-wise. The latter approach, in particular, can facilitate the development of novel molecules, uniquely designed to bind to the subject binding pockets.

The test compound may be screened as part of a library or a data base of molecules. Modulators of a binding pocket of the present invention may be identified by docking a computer representation of compounds from one or more database of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may tested for their capacity to fit spatially into a binding pocket. As used herein, the term “fits spatially” means that the three-dimensional structure of the test compound is accommodated geometrically in a cavity of a binding pocket. The test compound can then be considered to be a ligand.

A favourable geometric fit occurs when the surface area of the test compound is in close proximity with the surface area of the cavity of a binding pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding pocket.

If a model of the present invention is a computer model, the test compounds may be positioned in a binding pocket through computational docking. If, on the other hand, the model of the present invention is a structural model, the test compounds may be positioned in the binding pocket by, for example, manual docking.

As used herein the term “docking” refers to a process of placing a compound in close proximity with a binding pocket, or a process of finding low energy conformations of a test compound/binding pocket complex.

A screening method of the present invention may comprise the following steps:

(i) generating a computer model of a binding pocket using a crystal according to the invention;

(ii) docking a computer representation of a test compound with the computer model; and

(iii) analysing the fit of the compound in the binding pocket.

In an aspect of the invention, a method is provided comprising the following steps:

(a) docking a computer representation of a structure of a test compound into a computer representation of a binding pocket defined in accordance with the invention using a computer program, or by interactively moving the representation of the test compound into the representation of the binding pocket;

(b) characterizing the geometry and the complementary interactions formed between the atoms of the binding pocket and the compound; optionally

(c) searching libraries for molecular fragments which can fit into the empty space between the compound and the binding pocket and can be linked to the compound; and

(d) linking the fragments found in (c) to the compound and evaluating the new modified compound.

In an embodiment of the invention, a method is provided which comprises the following steps:

(a) docking a computer representation of a test compound from a computer data base with a computer representation of a selected binding pocket defined in accordance with the invention to define a complex;

(b) determining a conformation of the complex with a favorable fit and favourable complementary interactions; and

(c) identifying test compounds that best fit the selected binding pocket as potential modulators of a F-box protein or SCF complex comprising the binding pocket.

In another embodiment of the invention, a method is provided which comprises docking a computer representation of a selected binding pocket defined by the atomic interactions, atomic contacts, or structural coordinates in accordance with the invention to define a complex. In particular a method is provided comprising:

(a) docking a computer representation of a test compound from a computer database with a computer representation of a selected binding pocket defined by the atomic interactions, atomic contacts, or structural coordinates described herein;

(b) determining a conformation of the complex with a favorable fit and favourable complementary interactions; and

(c) identifying test compounds that best fit the selected binding pocket as potential modulators of the a F-box protein or SCF complex comprising the binding pocket

A model used in a screening method may comprise a binding pocket either alone or in association with one or more ligands and/or cofactors. For example, the model may comprise the binding pocket in association with a substrate (or analogue thereof), and/or modulator.

If the model comprises an unassociated binding pocket, then the selected site under investigation may be the binding pocket itself. The test compound may, for example, mimic a known ligand (e.g. substrate) for an F-box protein in order to interact with the binding pocket. The selected site may alternatively be another site on the F-box protein.

If the model comprises an associated binding pocket, for example a binding pocket in association with a substrate or ligand, the selected site may be the binding pocket or a site made up of the binding pocket and the complexed substrate or ligand, or a site on the substrate or ligand itself. The test compound may be investigated for its capacity to modulate the interaction with the associated molecule.

The screening methods described herein may be applied to a plurality of test compounds, to identify those that best fit the selected site. A test compound (or plurality of test compounds) may be selected on the basis of their similarity to a substrate or ligand for an F-box protein. For example, the screening method may comprise the following steps:

(i) generating a computer model of a binding pocket in complex with a substrate or ligand;

(ii) searching for a test compound with a similar three dimensional structure and/or similar chemical groups as the substrate or ligand; and

(iii) evaluating the fit of the test compound in the binding pocket.

Searching may be carried out using a database of computer representations of potential compounds, using methods known in the art.

The present invention also provides a method for designing ligands for F-box proteins or SCF complexes. It is well known in the art to use a screening method as described above to identify a test compound with promising fit, but then to use this test compound as a starting point to design a ligand with improved fit to the model. Such techniques are known as “structure-based ligand design” (See Kuntz et al., 1994, Acc. Chem. Res. 27:117; Guida, 1994, Current Opinion in Struc. Biol. 4: 777; and Colman, 1994, Current Opinion in Struc. Biol. 4: 868, for reviews of structure-based drug design and identification; and Kuntz et al 1982, J. Mol. Biol. 162:269; Kuntz et al., 1994, Acc. Chem. Res. 27: 117; Meng et al., 1992, J. Compt. Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG (Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Fuction, and Genetics 8:195); and, GRID (Goodford, 1985, J. Med. Chem. 28:849).

The method may comprise the following steps:

(i) docking a model of a test compound with a model of a binding pocket;

(ii) identifying one or more groups on the test compound which may be modified to improve their fit in the binding pocket;

(iii) replacing one or more identified groups to produce a modified test compound model; and

(iv) docking the modified test compound model with the model of the binding pocket.

Evaluation of fit may comprise the following steps:

(a) mapping chemical features of a test compound such as by hydrogen bond donors or acceptors, hydrophobic/lipophilic sites, positively ionizable sites, or negatively ionizable sites; and

(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the binding pocket. Preferably the group modifications involve the addition removal or replacement of substituents onto the test compound such that the substituents are positioned to collide or to bind preferentially with one or more amino acid residues that correspond to the key amino acid residues of the binding pocket.

If a modified test compound model has an improved fit, then it may bind to a binding pocket and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

The test compound may also be modified “in situ” (i.e. once docked into the potential binding pocket), enabling immediate evaluation of the effect of replacing selected groups. The computer representation of the test compound may be modified by deleting a chemical group or groups, or by adding a chemical group or groups. After each modification to a compound, the atoms of the modified compound and potential binding pocket can be shifted in conformation and the distance between the modulator and the binding pocket atoms may be scored on the basis of geometric fit and favourable complementary interactions between the molecules. This technique is described in detail in Molecular Simulations User Manual, 1995 in LUDI.

Examples of ligand building and/or searching computer programs include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known substrate or lignad. For example, in order to identify potential modulators of an F-box protein, a logical approach would be to start with a known ligand or substrate to produce a molecule which mimics the binding of the ligand or substrate. Such a molecule may, for example, act as a competitive inhibitor for the true substrate or ligand, or may bind so strongly that the interaction (and inhibition) is effectively irreversible.

Such a method may comprise the following steps:

(i) generating a computer model of a binding pocket in complex with a substrate or ligand;

(ii) replacing one or more groups on the ligand model to produce a modified substrate or ligand; and

(iii) evaluating the fit of the modified substrate or ligand in the binding pocket.

The replacement groups could be selected and replaced using a compound construction program which replaces computer representations of chemical groups with groups from a computer database, where the representations of the compounds are defined by structural coordinates.

In an embodiment, a screening method is provided for identifying a substrate or ligand of an F-box protein, comprising the step of using the structural coordinates of a CPD motif defined in relation to its spatial association with a binding pocket of the invention, to generate a compound that is capable of associating with the binding pocket.

In an embodiment of the invention, a screening method is provided for identifying a ligand of an F-box protein, in particular a cdc4 protein, comprising the step of using the structural coordinates of the CPD motif listed in Table 6 to generate a compound for associating with a binding pocket of an F-box protein, in particular a cdc4 protein as described herein. The following steps are employed in a particular method of the invention: (a) generating a computer representation of a CPD motif defined by its structural coordinates listed in Table 6; and (b) searching for molecules in a data base that are structurally or chemically similar to the defined CPD motif, using a searching computer program, or replacing portions of the CPD motif with similar chemical structures from a database using a compound building computer program.

A screening method is provided for identifying a ligand of an F-box protein, in particular a cdc4 protein, or a SCF complex comprising the step of using the structural coordinates of a binding pocket comprising a WD40 repeat or part thereof listed in Table 6 to generate a compound for associating with a F-box domain of an F-box protein. The following steps are employed in a particular method of the invention: (a) generating a computer representation of a binding pocket comprising a WD40 repeat region or part thereof defined by its structural coordinates listed in Table 6; and (b) searching for molecules in a data base that are structurally or chemically similar to the defined binding pocket using a searching computer program, or replacing portions of the binding pocket with structures from a database using a compound building computer program.

A screening method is provided for identifying a ligand of an F-box protein, in particular a cdc4 protein, of a SCF complex comprising the step of using the structural coordinates of a binding pocket comprising an F-box domain or part thereof, or helical linker listed in Table 6 to generate a compound for associating with a F-box domain or helical linker of an F-box protein. The following steps are employed in a particular method of the invention: (a) generating a computer representation of a binding pocket comprising a an F-box domain or part thereof, or helical linker defined by its structural coordinates listed in Table 4; and (b) searching for molecules in a data base that are structurally or chemically similar to the defined binding pocket using a searching computer program, or replacing portions of the binding pocket with structures from a database using a compound building computer program.

The screening methods of the present invention may be used to identify compounds or entities that associate with a molecule that associates with an F-box protein, in particular a cdc4 protein, or an SCF complex.

In an illustrative embodiment, the design of potential modulators or substrates for SCF complexes begins from the general perspective of shape complimentarity for an active site and substrate specificity subsites of the receptor, and a search algorithm is employed which is capable of scanning a database of small molecules of known three-dimensional structure for candidates which fit geometrically into the target protein site. It is not expected that the molecules found in the shape search will necessarily be leads themselves, since no evaluation of chemical interaction need necessarily be made during the initial search. Rather, it is anticipated that such candidates might act as the framework for further design, providing molecular skeletons to which appropriate atomic replacements can be made. Of course, the chemical complimentarity of these molecules can be evaluated, but it is expected that atom types will be changed to maximize the electrostatic, hydrogen bonding, and hydrophobic interactions with the receptor. Most algorithms of this type provide a method for finding a wide assortment of chemical structures that are complementary to the shape of a binding site of a subject molecule or complex. Each of a set of small molecules from a particular data-base, such as the Cambridge Crystallographic Data Bank (CCDB) (Allen et al. (1973) J. Chem. Doc. 13: 119), is individually docked to the binding pocket of the invention, in a number of geometrically permissible orientations with use of a docking algorithm. In a preferred embodiment, a set of computer algorithms called DOCK, can be used to characterize the shape of invaginations and grooves that form active sites and recognition surfaces of a subject molecule or complex (Kuntz et al. (1982) J. Mol. Biol 161: 269-288). The program can also search a database of small molecules for templates whose shapes are complementary to particular binding pockets or sites of a receptor (DesJarlais et al. (1988) J Med Chem 31: 722-729). These templates normally require modification to achieve good chemical and electrostatic interactions (DesJarlais et al. (1989) ACS Symp Ser 413: 60-69). However, the program has been shown to position accurately known cofactors for ligands based on shape constraints alone.

The orientations are evaluated for goodness-of-fit and the best are kept for further examination using molecular mechanics programs, such as AMBER or CHARMM. Such algorithms have previously proven successful in finding a variety of molecules that are complementary in shape to a given binding site of a molecule or complex, and have been shown to have several attractive features. First, such algorithms can retrieve a remarkable diversity of molecular architectures. Second, the best structures have, in previous applications to other proteins, demonstrated impressive shape complementarity over an extended surface area. Third, the overall approach appears to be quite robust with respect to small uncertainties in positioning of the candidate atoms.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) on the molecular surface of the binding site. GRID hence provides a tool for suggesting modifications to known ligands that might enhance binding. It may be anticipated that some of the sites discerned by GRID as regions of high affinity correspond to “pharmacophoric patterns” determined inferentially from a series of known ligands. As used herein, a pharmacophoric pattern is a geometric arrangement of features of the anticipated ligand that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20); however, the constraint of steric and “chemical” fit in the putative (and possibly unknown) binding pocket or site is ignored. Goodsell and Olson (1990, Proteins: Struct Funct Genet 8:195-202) have used the Metropolis (simulated annealing) algorithm to dock a single known ligand into a target protein. They allow torsional flexibility in the ligand and use GRID interaction energy maps as rapid lookup tables for computing approximate interaction energies. Given the large number of degrees of freedom available to the ligand, the Metropolis algorithm is time-consuming and is unsuited to searching a candidate database of a few thousand small molecules.

Yet a further embodiment of the present invention utilizes a computer algorithm such as CLIX which searches such databases as CCDB for small molecules which can be oriented in a binding pocket or site in a way that is both sterically acceptable and has a high likelihood of achieving favorable chemical interactions between the candidate molecule and the surrounding amino acid residues. The method is based on characterizing a binding pocket in terms of an ensemble of favorable binding positions for different chemical groups and then searching for orientations of the candidate molecules that cause maximum spatial coincidence of individual candidate chemical groups with members of the ensemble. The current availability of computer power dictates that a computer-based search for novel ligands follows a breadth-first strategy. A breadth-first strategy aims to reduce progressively the size of the potential candidate search space by the application of increasingly stringent criteria, as opposed to a depth-first strategy wherein a maximally detailed analysis of one candidate is performed before proceeding to the next. CLIX conforms to this strategy in that its analysis of binding is rudimentary—it seeks to satisfy the necessary conditions of steric fit and of having individual groups in “correct” places for bonding, without imposing the sufficient condition that favorable bonding interactions actually occur. A ranked “shortlist” of molecules, in their favored orientations, is produced which can then be examined on a molecule-by-molecule basis, using computer graphics and more sophisticated molecular modeling techniques. CLIX is also capable of suggesting changes to the substituent chemical groups of the candidate molecules that might enhance binding.

The algorithmic details of CLIX is described in Lawerence et al. (1992) Proteins 12:31-41, and the CLIX algorithm can be summarized as follows. The GRID program is used to determine discrete favorable interaction positions (termed target sites) in the binding pocket or site of the protein for a wide variety of representative chemical groups. For each candidate ligand in the CCDB an exhaustive attempt is made to make coincident, in a spatial sense in the binding site of the protein, a pair of the candidate's substituent chemical groups with a pair of corresponding favorable interaction sites proposed by GRID. All possible combinations of pairs of ligand groups with pairs of GRID sites are considered during this procedure. Upon locating such coincidence, the program rotates the candidate ligand about the two pairs of groups and checks for steric hindrance and coincidence of other candidate atomic groups with appropriate target sites. Particular candidate/orientation combinations that are good geometric fits in the binding site and show sufficient coincidence of atomic groups with GRID sites are retained.

Consistent with the breadth-first strategy, this approach involves simplifying assumptions. Rigid protein and small molecule geometry is maintained throughout. As a first approximation rigid geometry is acceptable as the energy minimized coordinates of a deduced structure, describe an energy minimum for the molecule, albeit a local one. If the surface residues of the site of interest are not involved in crystal contacts then the crystal configuration of those residues is used merely as a starting point for energy minimization, and potential solution structures for those residues determined. The deduced structure should reasonably mimic the mean solution configuration.

A further assumption implicit in CLIX is that the potential ligand, when introduced into the binding pocket or site of a receptor, does not induce change in the protein's stereochemistry or partial charge distribution and so alter the basis on which the GRID interaction energy maps were computed. It must also be stressed that the interaction sites predicted by GRID are used in a positional and type sense only, i.e., when a candidate atomic group is placed at a site predicted as favorable by GRID, no check is made to ensure that the bond geometry, the state of protonation, or the partial charge distribution favors a strong interaction between the protein and that group. Such detailed analysis should form part of more advanced modeling of candidates identified in the CLIX shortlist.

Yet another embodiment of a computer-assisted molecular design method for identifying ligands of a binding pocket of the invention comprises the de novo synthesis of potential ligands by algorithmic connection of small molecular fragments that will exhibit the desired structural and electrostatic complementarity with an active site or binding pocket of the receptor. The methodology employs a large template set of small molecules with are iteratively pieced together in a model of a binding pocket. Each stage of ligand growth is evaluated according to a molecular mechanics-based energy function, which considers van der Waals and coulombic interactions, internal strain energy of the lengthening ligand, and desolvation of both ligand and receptor. The search space can be managed by use of a data tree that is kept under control by pruning according to the binding criteria.

In an illustrative embodiment, the search space is limited to consider only amino acids and amino acid analogs as the molecular building blocks. Such a methodology generally employs a large template set of amino acid conformations, though need not be restricted to just the 20 natural amino acids, as it can easily be extended to include other related fragments of interest to the medicinal chemist, e.g. amino acid analogs. The putative ligands that result from this construction method are peptides and peptide-like compounds rather than the small organic molecules that are typically the goal of drug design research. The appeal of the peptide building approach is not that peptides are preferable to organics as potential pharmaceutical agents, but rather that: (1) they can be generated relatively rapidly de novo; (2) their energetics can be studied by well-parameterized force field methods; (3) they are much easier to synthesize than are most organics; and (4) they can be used in a variety of ways, for peptidomimetic ligand design, protein-protein binding studies, and even as shape templates in the more commonly used 3D organic database search approach described above.

Such a de novo peptide design method has been incorporated in a software package called GROW (Moon et al. (1991) Proteins 11:314-328). In a typical design session, standard interactive graphical modeling methods are employed to define the structural environment in which GROW is to operate. For instance, environment could be an active site binding pocket of an F-box protein, or it could be a set of features on the protein's surface to which the user wishes to bind a peptide-like molecule. The GROW program then operates to generate a set of potential ligand molecules. Interactive modeling methods then come into play again, for examination of the resulting molecules, and for selection of one or more of them for further refinement.

To illustrate, GROW operates on an atomic coordinate file generated by the user in the interactive modeling session, such as the coordinates provided in Table 4, or the coordinates of a binding pocket or active site as described in Tables 2 and 4 plus a small fragment (e.g., an acetyl group) positioned in the active site to provide a starting point for peptide growth. These are referred to as “site” atoms and “seed” atoms, respectively. A second file provided by the user contains a number of control parameters to guide the peptide growth (Moon et al. (1991) Proteins 11:314-328).

The operation of the GROW algorithm is conceptually fairly simple. GROW proceeds in an iterative fashion, to systematically attach to the seed fragment each amino acid template in a large preconstructed library of amino acid conformations. When a template has been attached, it is scored for goodness-of-fit to the receptor site or binding pocket, and then the next template in the library is attached to the seed. After all the templates have been tested, only the highest scoring ones are retained for the next level of growth. This procedure is repeated for the second growth level; each library template is attached in turn to each of the bonded seed/amino acid molecules that were retained from the first step, and then scored. Again, only the best of the bonded seed/dipeptide molecules that result are retained for the third level of growth. The growth of peptides can proceed in the N-to-C direction only, the reverse direction only, or in alternating directions, depending on the initial control specifications supplied by the user. Successive growth levels therefore generate peptides that are lengthened by one residue. The procedure terminates when the user-defined peptide length has been reached, at which point the user can select from the constructed peptides those to be studied further. The resulting data provided by the GROW procedure includes not only residue sequences and scores, but also atomic coordinates of the peptides, related directly to the coordinate system of the binding site atoms.

In yet another embodiment, potential pharmacophoric compounds can be determined using a method based on an energy minimization-quenched molecular dynamics algorithm for determining energetically favorable positions of functional groups in the binding pockets of the invention. The method can aid in the design of molecules that incorporate such functional groups by modification of known ligands or de novo construction.

For example, the multiple copy simultaneous search method (MCSS) described by Miranker et al. (1991) Proteins 11: 29-34 may be employed. To determine and characterize a local minima of a functional group in the forcefield of the protein, multiple copies of selected functional groups are first distributed in a binding pocket of interest on the F-box protein. Energy minimization of these copies by molecular mechanics or quenched dynamics yields the distinct local minima. The neighborhood of these minima can then be explored by a grid search or by constrained minimization. In one embodiment, the MCSS method uses the classical time dependent Hartee (TDH) approximation to simultaneously minimize or quench many identical groups in the forcefield of the protein.

Implementation of the MCSS algorithm requires a choice of functional groups and a molecular mechanics model for each of them. Groups must be simple enough to be easily characterized and manipulated (3-6 atoms, few or no dihedral degrees of freedom), yet complex enough to approximate the steric and electrostatic interactions that the functional group would have in binding to the pocket or site of interest in the F-box protein. A preferred set is, for example, one in which most organic molecules can be described as a collection of such groups (Patai's Guide to the Chemistry of Functional Groups, ed. S. Patai (New York: John Wiley, and Sons, (1989)). This includes fragments such as acetonitrile, methanol, acetate, methyl ammonium, dimethyl ether, methane, and acetaldehyde.

Determination of the local energy minima in the binding pocket or site requires that many starting positions be sampled. This can be achieved by distributing, for example, 1,000-5,000 groups at random inside a sphere centered on the binding site; only the space not occupied by the protein needs to be considered. If the interaction energy of a particular group at a certain location with the protein is more positive than a given cut-off (e.g. 5.0 kcal/mole) the group is discarded from that site. Given the set of starting positions, all the fragments are minimized simultaneously by use of the TDH approximation (Elber et al. (1990) J Am Chem Soc 112: 9161-9175). In this method, the forces on each fragment consist of its internal forces and those due to the protein. The essential element of this method is that the interactions between the fragments are omitted and the forces on the protein are normalized to those due to a single fragment. In this way simultaneous minimization or dynamics of any number of functional groups in the field of a single protein can be performed.

Minimization is performed successively on subsets of, for example 100, of the randomly placed groups. After a certain number of step intervals, such as 1,000 intervals, the results can be examined to eliminate groups converging to the same minimum. This process is repeated until minimization is complete (e.g. RMS gradient of 0.01 kcal/mole/C). Thus the resulting energy minimized set of molecules comprises what amounts to a set of disconnected fragments in three dimensions representing potential pharmacophores.

The next step then is to connect the pharmacophoric pieces with spacers assembled from small chemical entities (atoms, chains, or ring moieties). In a preferred embodiment, each of the disconnected can be linked in space to generate a single molecule using such computer programs as, for example, NEWLEAD (Tschinke et al. (1993) J Med Chem 36: 3863,3870). The procedure adopted by NEWLEAD executes the following sequence of commands: (1) connect two isolated moieties, (2) retain the intermediate solutions for further processing, (3) repeat the above steps for each of the intermediate solutions until no disconnected units are found, and (4) output the final solutions, each of which is a single molecule. Such a program can use for example, three types of spacers: library spacers, single-atom spacers, and fuse-ring spacers. The library spacers are optimized structures of small molecules such as ethylene, benzene and methylamide. The output produced by programs such as NEWLEAD consist of a set of molecules containing the original fragments now connected by spacers. The atoms belonging to the input fragments maintain their original orientations in space. The molecules are chemically plausible because of the simple makeup of the spacers and functional groups, and energetically acceptable because of the rejection of solutions with van-der Waals radii violations.

Compounds and entities (e.g. ligands) of F-box proteins, in particular cdc4 proteins, or SCF complexes identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

Test compounds and ligands which are identified using a crystal or model of the present invention can be screened in assays such as those well known in the art. Screening may be for example in vitro, in cell culture, and/or in vivo. Biological screening assays preferably centre on activity-based response models, binding assays (which measure how well a compound binds to a binding pocket of a receptor), and bacterial, yeast, and animal cell lines (which measure the biological effect of a compound in a cell). The assays may be automated for high throughput screening in which large numbers of compounds can be tested to identify compounds with the desired activity. The biological assay may also be an assay for the binding activity of a compound that selectively binds to the binding pocket compared to other receptors.

Ligands/Compounds Identified by Screening Methods

The present invention provides a ligand or compound identified by a screening method of the present invention. A ligand or compound may have been designed rationally by using a model according to the present invention. A ligand or compound identified using the screening methods of the invention may specifically associate with a target compound, or part thereof (e.g. a binding pocket). In the present invention the target compound may be the F-box protein or SCF complex or part thereof, or a molecule that is capable of associating with an F-box protein or SCF complex or part thereof (for example a substrate).

A ligand or compound identified using a screening method of the invention may act as a “modulator”, i.e. a compound which affects the activity of an F-box protein or SCF complex. A modulator may reduce, enhance or alter the biological function of an F-box protein or an SCF E3 ubiquitin ligase. For example a modulator may modulate the capacity of the F-box protein or an SCF E3 ubiquitin ligase to interact with its substrate. An alteration in biological function may be characterised by a change in specificity. For example, a modulator may cause the F-box protein to interact with a different substrate. In order to exert its function, the modulator commonly binds to a binding pocket.

A “modulator” which is capable of reducing the biological function of the enzyme may also be known as an inhibitor. Preferably an inhibitor reduces or blocks the capacity of the F-box protein or an SCF E3 ubiquitin ligase to interact with its substrate thus reducing or blocking ubiquitination of the substrate. The inhibitor may mimic the binding of a substrate, for example, it may be a substrate analogue. A substrate analogue may be designed by considering the interactions between the substrate and the F-box protein or an SCF E3 ubiquitin ligase (for example, by using information derivable from the crystal of the invention) and specifically altering one or more groups (as described above).

The present invention also provides a method for modulating the activity of an F-box protein, in particular a cdc4 protein, using a modulator according to the present invention. The invention also provides a method for modulating (e.g. potentiating or inhibiting) ubiquitination of a substrate by an SCF E3 ubiquitin ligase, by potentiating or inhibiting the substrate binding pocket of the ligase. Inhibition of ubiquitination of a substrate may decrease signaling and inhibit cellular processes that may be involved in disease. It would be possible to monitor cellular processes following such treatments by a number of methods known in the art.

A modulator may be an agonist, partial agonist, partial inverse agonist or antagonist of an F-box protein.

As mentioned above, a substrate or an identified ligand may act as a ligand model (for example, a template) for the development of other compounds. A modulator may be a mimetic of a substrate or ligand.

Like the test compound (see above) a modulator may be one or a variety of different sorts of molecule. (See examples herein.) A modulator may be an endogenous physiological compound, or it may be a natural or synthetic compound. The term “modulator” also refers to a chemically modified ligand or substrate.

The technique suitable for preparing a modulator will depend on its chemical nature. For example, peptides can be synthesized by solid phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Once cleaved from the resin, the peptide may be purified by preparative high performance liquid chromatography (e.g., Creighton (1983) Proteins Structures and Molecular Principles, WH Freeman and Co, New York N.Y.). The composition of the synthetic peptides may be confirmed by amino acid analysis or sequencing (e.g., the Edman degradation procedure; Creighton, supra).

If a modulator is a nucleotide, or a polypeptide expressable therefrom, it may be synthesized, in whole or in part, using chemical methods well known in the art (see Caruthers M H et al (1980) Nuc Acids Res Symp Ser 215-23, Horn T et al (1980) Nuc Acids Res Symp Ser 225-232), or it may be prepared using recombinant techniques well known in the art.

Organic compounds may be prepared by organic synthetic methods described in references such as March, 1994, Advanced Organic Chemistry: Reactions, Mechanisms, and Structure, New York, McGraw Hill.

The invention also relates to classes of modulators of F-box proteins, in particular cdc4 proteins based on the structure and shape of a substrate or component thereof, defined in relation to the substrate's spatial association with a crystal structure of the invention or part thereof.

A class of modulators may comprise a compound containing a structure of a CPD motif. In particular, the modulators can comprise a CPD motif having the structural coordinates of the CPD motif in the active site binding pocket of an F-box protein. In an embodiment, a modulator comprises the structural coordinates of a CPD motif having the structural coordinates listed in Table 6.

The invention contemplates all optical isomers and racemic forms of the modulators of the invention.

Pharmaceutical Composition

The present invention also provides for the use of a modulator according to the invention, in the manufacture of a medicament to treat and/or prevent a disease in a mammalian patient. There is also provided a pharmaceutical composition comprising such a modulator and a method of treating and/or preventing a disease comprising the step of administering such a modulator or pharmaceutical composition to a subject, preferably a mammalian patient.

The pharmaceutical compositions may be for human or animal usage in human and veterinary medicine and will typically comprise a pharmaceutically acceptable carrier, diluent, excipient, adjuvant or combination thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may also be used.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, gel, hydrogel, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For buccal or sublingual administration the compositions may be administered in the form of tablets or lozenges which can be formulated in a conventional manner.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

As indicated, a therapeutic agent (e.g. modulator) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Therapeutic administration of polypeptide modulators may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses.

Applications

The invention further provides a method of treating a mammal, the method comprising administering to a mammal a modulator or pharmaceutical composition of the present invention.

In particular, the invention contemplates a method of treating or preventing a condition or disease associated with an F-box protein or SCF complex in a cellular organism, comprising:

(a) administering a modulator of the invention in an acceptable pharmaceutical preparation; and

(b) activating or inhibiting an F-box protein or SCF complex or their interaction with a substrate to treat or prevent the disease.

The invention provides for the use of a modulator identified by the methods of the invention in the preparation of a medicament to treat or prevent a disease in a cellular organism. Use of modulators of the invention to manufacture a medicament is also provided.

Typically, a physician will determine the actual dosage of a modulator or pharmaceutical composition of the invention that will be most suitable for an individual subject and it will vary with the age, weight and response of the particular patient and severity of the condition. There can, of course, be individual instances where higher or lower dosage ranges are merited.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. By way of example, the pharmaceutical composition of the present invention may be administered in accordance with a regimen of 1 to 10 times per day, such as once or twice per day.

For oral and parenteral administration to human patients, the daily dosage level of the agent may be in single or divided doses.

The modulators and compositions of the invention may be useful in the prevention and treatment of conditions involving aberrant F-box proteins or SCF complexes. In particular the modulators and compositions may be useful in treating cancer or Alzheimer's Disease.

Conditions which may be prevented or treated in accordance with the invention include but are not limited to lymphoproliferative conditions, and malignant and pre-malignant conditions. Malignant and pre-malignant conditions may include solid tumors, B cell lymphomas, chronic lymphocytic leukemia, chronic myelogenous leukemia, prostate hypertrophy, Hirschsprung disease, glioblastoma, breast and ovarian cancer, adenocarcinoma of the salivary gland, premyelocytic leukemia, prostate cancer, multiple endocrine neoplasia type IIA and IIB, medullary thyroid carcinoma, papillary carcinoma, papillary renal carcinoma, hepatocellular carcinoma, gastrointestinal stromal tumors, sporadic mastocytosis, acute myeloid leukemia, large cell lymphoma or Alk lymphoma, chronic myeloid leukemia, hematological/solid tumors, papillary thyroid carcinoma, stem cell leukemia/lymphoma syndrome, acute myelogenous leukemia, osteosarcoma, multiple myeloma, preneoplastic liver foci, and resistance to chemotherapy.

Modulators and compositions of the invention may be used to restore function to a mutant F-box protein, in particular a mutant cdc4 polypeptide. Modulators and compositions of the invention, in particular inhibitors may also have utility in treating diseases associated with F-box mutations, in particular cdc4 polypeptide mutations, in combination with other cancer mutations, Notch pathway mutations or presenilin mutations.

A modulator of the invention may be used to promote binding of a substrate to a SCF complex. In an embodiment a modulator that associates (preferably with high affinity) with a binding pocket of a SCF complex as described herein, is linked to an agent that binds to a substrate to be ubiquitinated by a SCF complex. A modulator-agent-substrate complex where the modulator is derived from a binding pocket of an F-box protein as described herein may be used in treating diseases associated with a mutant F-box protein.

Therapeutic efficacy and toxicity of compositions and modulators of the invention may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions that exhibit large therapeutic indices are preferred.

The invention will now be illustrated by the following non-limiting examples:

### EXAMPLE 1

The following methods were used in the investigation described in the example:

Methods

Cloning, Protein Expression and Purification

The Cdc4 fragment employed for crystalization, which is deleted for terminal residues 1 to 262 and 745 to 779, extends from the beginning of the F-box domain to the end of the WD40 repeat domain. The N-terminal deletion removes a poorly conserved sequence of 226 amino acids and a conserved element of approximately 40 residues termed the D-domain that immediately precedes the fbox domain and that has been implicated in molecular multimerization. The C terminal deletion removes residues not conserved amongst different Cdc4 homologues. Both Skp1 and Cdc4 were engineered to remove flexible loops, namely residues 36-55 in Skp1 and residues 601 to 604 and 609 to 624 in Cdc4.

A PCR product containing CDC4(263-744) was cloned into the EheI(SfoI) and BamH1 sites of pPROEX HTb. In parallel, a PCR product containing SKP1Δ37-64 was cloned into the NdeI and BamHI sites of pGEX2T-TEV. An SspI GST-SKP1-containing fragment from this construct was cloned into the StuI site of the Cdc4 construct described above such that CDC4 and SKP1 were in opposite orientations. A non-homologous region in CDC4 encoding amino acids 602-624 was then replaced by the DNA sequence GGCGAACTG [SEQ ID NO. 39], which encodes the shorter peptide sequence Gly-Glu-Leu.

The Cdc4/skp1 complex was expressed in E. coli B934 (DE3) cells grown in minimal media suplemented with a mixture of selenomethionine (40 ug/ml) and methionine (0.4 ug/ml). Cells were induced with 0.2 mM isopropyl-β-D-thiogalactopyranoside (IPTG) at 15° C. overnight. Cell pellets were resuspended in 50 mM hepes pH 7.5, 500 mM NaCl, 10% glycerol, and 5 mM Imidazole, lysed with a cell homogenizer (Emulsiflex C-5, Avistin) followed by a 20 sec sonication (vibra cell, Betatec). The lysate was then clarified by centrifugation at 65 000×g for 40 min. The supernatant was loaded onto a 5 ml metal chelating column (Pharmacia) and eluted in high imidazole. This fraction was loaded onto a glutathione-sepharose column (Pharmacia) and the bound complex was eluted by overnight digestion with TEV protease (Canadian Life). Eluted protein was dialysed to remove DTT and EDTA and reloaded onto a metal chelating column. The flow through containing the complex was concentrated and applied to a Superdex S 75 gel filtration column (Pharmacia). Fractions containing the complex were concentrated in a buffer containing 10 mM hepes pH 7.5, 250 mM NaCl, and 1 m M DTT.

Crystallization, Data Collection, and Structure Determination

Hanging drops containing 1 μl of 21 mg/ml protein plus 1.2 molar equivilents of the CPD peptide sequence were mixed with equal volumes of reservoir buffer containing 0.1 M Tris pH 8.5, and 1.5 M ammonium sulphate. Crystals were flash frozen in reservoir buffer supplemented with 15% glycerol. Crystals of the space group P3₂, (α=107.7 Å, b=107.7 Å, c=168.3 Å, α=γ=90°, β=120°), with two molecules of the complex in the asymmetric unit were obtained at 20° C. A Multiple Anomalous Dispersion (MAD) experiment was performed on a frozen crystal at the Advanced Photon Source (Argonne, Ill.) (APS) beamline BM 14-B and BM 14-D(λ1=0.9798 Å, λ2=0.9800 Å, λ3=0.9000 Å) using a Quantum 4 ADSC CCD detector. Data processing and reduction was carried out with the HKL program suite (Otwinowski and Minor, 1997). The programs SHARP (de La Fortelle and Bricogne, 1997) and SnB (Miller et al., 1994) were used in combination to locate and refine 19 of the 22 Se sites. Following density modification with Solomon (Abrahams and Leslie, 1996), a partial model was generated using 0 (Jones et al., 1991) and refined using CNS (Brunger et al., 1998) to a working R value of 24.09% and a free R value of 28.71%. Pertinent statistics for data collection and refinement are shown in Table 1.

The increased order of the second CPDs may be due to a crystal packing interaction involving the c-terminus of the CPD. While the main chain termini of the second CPD are discernable (FIG. 3e), the precise backbone and side chain conformations for the P−2 Leu, P−3 Gly, P+4 Ser, and P+5 Gly are less reliably determined.

Mutagenesis

Point mutants were obtained by a PCR-based approach using oligos provided in supplementary information and Pfu polymerase (Stratagene). Once verified by sequencing the mutants were sub-cloned into the appropriate vectors as listed in the supplementary information. Alanine insertion mutations were obtained using the Kunkel method (ref) and then sub-cloned into the vectors indicated in the supplementary information.

Shuffle Experiments

All mutants on a TRP1 ARS CEN plasmid were transformed into a cdc4Δ strain (MT 1259) containing a wildtype copy of CDC4 on a URA3 ARS CEN plasmid. Cells were plated on either Trp⁻Ura⁻ or 5-FOA medium for 2 days at 30° C. Viable cells on 5-FOA were grown in Trp medium and transformed with either wild type GAL1-SIC1, GAL1-SIC T45A, or GAL1-SIC T33V on a LEU2 ARS CEN pasmid. Cells were then plated on Leu- Trp-plates containing either glucose or galactose and incubated for 2 days at 30° C.

Sic1-Cdc4 Interactions.

Bacterially expressed His6-Sic1 was phosphorylated with Cln2-Cdc28 kinase purified from baculovirus infected Sf9 cells as described before (Nature paper). 1 ug of WT or mutant Cdc4-GST-Skp1, immoblized on GSH-Sepharose resin, was incubated with 0.5 ug phospho-Sic1 at 4 C for 1 h and washed 4 times. Captured complexes were resolved on SDS-PAGE and Sic1 visualized by anti-Sic1 Western blotting and ECL. For IEF-2D analysis, several Sic1 phosphorylation reactions were carried out for different time periods to obtain a spectrum of Sic1 that were phosphorylated at different numbers of its nine CDK sites. This pool of phospho-Sic1 (2.5 ug) was incubated with 5 μg of WT or mutant Cdc4-GST-Skp1 as described above. Different phosphorylation states of Sic1 were separated by denaturing isoelectric focusing (IEF)-2D gel electrophoresis and visualized by anti-Sic1 Western blotting and ECL. IEF was performed using pH 3-10NL Immobiline gel strips and IPGphore IEF system (Amersham pharmacia).

Results

The x-ray crystal structure presented herein consists of a ternary complex of yeast Skp1 bound to a fragment of Cdc4, and a 9mer high affinity CPD phosphopeptide (FIG. 2). The Cdc4 fragment, which is deleted for terminal residues 1 to 262 and 745 to 779, extends from the beginning of the F-box domain to the end of the WD40 repeat domain.

Skp1-Cdc4 Fbox: Skp1 forms an elongated structure with a mixed α/β topology identical to that reported for human Skp1 (Schulman et al, 2000). The topology consists of a three-strand (denoted β1 to β3) β-sheet and eight α-helices, denoted α1 to α8 (FIG. 2a). The structure of Cdc4 from its amino terminus consists of an F-box domain, an α-helical extension or linker, and a WD40 repeat domain (FIG. 2a,b). The F-box domain comprises five a helices (denoted α0 to α4)\tilde{.} This topology differs slightly from that reported for the F-box domain of hSkp2 (Schulman et al, 2000), which consists of a loop region L1 and three helices denoted α1 to α3. Helix α0 in Cdc4 corresponds most closely in sequence and position to the loop region L1 of Skp2 while a half turn remnant of helix α4 is discernable in the transition sequence between the Skp2 F-box and Leucine Repeat domains. As observed in the Skp1-Skp2 complex, Skp1 and the F-box domain of Cdc4 associate by the interdigiation of helixes α0 to α3 of Cdc4 with helices α5 to α8 of Skp1. This mode of inter-domain association is characterized by a common and continuous hydrophobic core that spans the two protein domains.

Cdc4 helical linker and WD40 domain: Following the F-box domain of Cdc4 is a helical extension that forms a structured bridge to the WD40 repeat domain. The helical extension consists of two α-helices α5 and α6 that together with helices α3 and α4 of the F-box domain form a stalk and pedestal like structure that connects and orients the WD40 domain (FIG. 2c).

Eight copies of the WD40 repeat motif in Cdc4 form an 8 blade β-propeller structure. Each blade, composed of 4 anti-parallel β-strands, is related by 8-fold pseudo symmetry about a central axis (FIG. 2b). As first shown for G-protein gamma subunit (Sondek 1996), the WD40 repeat motif of approximately 40 amino acids composes the outer β-strand of one propeller blade and the inner three strands of the adjacent blade. A continuous circular arrangement of blades is formed by the association of the first and last WD40 repeat motifs to form the 8^{th} propeller blade. Interestingly, a 7 β-propeller blade structure was anticipated for Cdc4 and its orthologues (and generally all WD40 repeat F-box adaptors), which is attributable in part to the cryptic nature of the 8th WD40 repeat motif (FIG. 1). Based on the structure based sequence alignment in FIG. 1, it is predicted that the other WD40 class of F-box adaptor proteins (i.e. the Met30 orthologues and βTRCP orthologues) will form 7-blade β-propeller structures.

The WD40 repeat domain forms a disk like structure characterized by a cavity in the middle and two opposing circular surfaces of slightly different size. The smaller of the two surfaces composes the CPD binding site. On the bottom surface is anchored helix α6 of the helical extension, which inserts obliquely between propeller blades β7 and β8. Interestingly, β-propeller blade 2 consists of 5 β-strands. The outermost strand of this blade, denoted β9¹, is non-standard and arises from an amino acid insert in the connecting loop between α-strands 12 and 13. Strand β9¹, forms a parallel arrangement with strand β9, which differs from the anti parallel architecture of all other 1-strand elements in the WD40 domain structure. A large insert in the β12-β13 linker is absent from dr, ce, hu, mu Cdc4 homologues suggesting that a 5 \tilde{β} strand propeller blade 2 is unique to the fungal homologues.

A fixed orientation between the F-box domain and WD40 domain of Cdc4 is maintained largely through the integrity of the stalk like helix α6 of the helical extension (FIG. 2c). Helix α6 is 30 Å in length, and is anchored at its N-terminus to the hydrophobic core of the F-box/helical extension and at its C-terminus to the hydrophobic core of the WD40 repeat domain. In contrast to the intermolecular connection between Skp1 and the F-box domain, the connection between the F-box domain and WD40 repeat domain appears less rigidly structured.

At its amino terminus, helix α6 anchors to the F-box through hydrophobic interactions involving α6 residues Phe 355 and Leu 356 and F-box residues Ile 295, and Ile 296, Leu 315, Trp 316, and Leu 319 (FIG. 2c). Helix α5 packs along side the base of helix α6 opposite to the F-box domain through hydrophobic packing interactions involving Tyr342, Leu 338 and Leu 334. At its C-terminus, helix α6 anchors through hydrophobic interactions involving residues Trp 365 and Ile 364 with WD repeat residues Val 687, Ile 696, Leu 726 and Phe 743 in β-propeller blades 7 and 8. Asn 364 of helix α6 also forms a tight hydrogen bond interaction with the backbone carbonyl group of Phe 743 in propeller blade 8. The noted interactions (with the exception of interactions involving helix α5) involve residues that are conserved across most WD40 F-box adaptor proteins including the Met30 orthologues and β-TRCP orthologues, which suggests that the linkage between WD40 and F-box domains are similarly structured in these proteins. Helix α6 in β-TRCP, however, appears to be one α-helical turn longer (FIG. 1).

Outside of stalk helix α6, only two close contacts (<3.5 Å) are observed between the WD40 repeat domain and other regions of Cdc4. These contacts consist of hydrogen bonds between Asn684 and Arg700 in the loop regions of propeller blade 7 with Glu 323 in the α4-α5 linker of the helical extension. Both, hydrogen bonds are maintained in the two Cdc4 molecules of the crystal asymmetric unit but all three residues are poorly conserved amongst Cdc4 orthologues (FIG. 1). The lack of additional stabilizing interactions suggests that the F-box/WD40 domain linkage is not exceedingly rigid, and indeed, the WD40 domain in the two molecules of the asymmetric unit differ relative to their F-box domains by a 5 degree rotation about helix α6.

WD40 domain phosphopeptide recognition: A nine-mer CPD consisting of the sequence acetyl-Gly,Leu,Leu,pThr,Pro,Pro,Gln,Ser,Gly-amide [SEQ ID NO.40] is bound to the front face of the WD40 domain of Cdc4. In the two WD40 repeat domain/CPD complexes of the crystal asymmetric unit, a central core of 4 CPD residues corresponding to the sequence Leu, pThr, Pro, Pro [SEQ ID NO.41] is well ordered.

These residues have been modeled unambiguously in unbiased experimental electron density maps (FIG. 3e). Interpretable electron density is also apparent for the P−2 Leu, P−3 Gly, P+3 Gln, P+4 Ser, and P+5Gly positions of the second CPD (no interpretable electron density is apparent for these residues in the first CPD). The CPD binds in an extended manner across β-propeller blade 2 with the N-terminus oriented towards the central cavity of the WD40 repeat domain and the C-terminus oriented towards the outer rim. The CPD binding surface of Cdc4 is composed of invariant and highly conserved residues from β-propeller blades 1 to 6 and 8 and represents the most conserved part of the WD40 repeat domain surface (FIG. 3a,c).

Cdc4 displays an absolute requirement for phosphorylation at Ser or Thr at the P−0 position of the CPD. In the crystal structure, the P0 pThr phosphate group is coordinated by an intricate network of electrostatic interactions and hydrogen bonds involving residues absolutely conserved across all Cdc4 orthologues (FIG. 3c). The P0 phosphate group forms direct electrostatic interactions with the guanidinium groups of Arg 485, Arg 467, and Arg 534 and a direct hydrogen bond with the side chain of Tyr 548. The side chain of Tyr 548 is coordinated by stacking interactions with the guanidinium group of Arg 572, which in turn is coordinated by a hydrogen bond to the side chain of Tyr 574. Although Cdc4 shows a strong (6 fold) preference for pThr over pSer, the structural basis for this selectivity is not obvious. In the crystal structure, the Cy methyl group of Thr is directed towards solvent and does not make contact with the CPD binding surface of Cdc4. This binding preference may be due to the greater side chain rotational stability arising from the Thr β-branch structure.

Cdc4 displays an absolute requirement for proline in the p+1 CPD position. In the crystal structure, the P+1 proline side chain projects into a three-sided pocket on the CPD binding surface. The side chain of Trp 426 forms one side of the pocket and packs in a coplanar manner with the P+1 proline side chain. On its other side, the Trp 426 side chain packs tightly against the side chain of Thr 386. The opposite side of the P+1 binding pocket is formed by the side chain of Arg 485. Arg 485 coordinates the P+1 Proline through van der Wals side chain interactions and through a direct hydrogen bond to the Proline backbone carbonyl group. This represents the sole direct hydrogen bond interaction between Cdc4 and the CPD main chain. The side chains of Thr 441 and Thr 465 define the remaining side of the P+1 Proline binding pocket, with the Cy side chain groups composing a hydrophobic surface. The hydroxyl groups of Thr 441 and 465 orient away from the P+1 binding pocket, where they are well placed to influence binding specificity for CPD residues C-terminal to the P+1 position. Unlike Trp 426, Thr 386 and Arg 485, which are invariant amongst the Cdc4 orthologues, Thr 441 and Thr 465 are substituted with Ile in the S. pombe Cdc4 orthologue Pop1. The modeling studies suggest that this substitution has no effect on the P+1 binding pocket but may perturb CPD binding specificity C-terminal to the P+1 positions through steric effects (Ile is bigger than Thr) and by increasing the hydrophobic character of the surface.

Cdc4 displays a strong preference for the hydrophobic residues Leu, Ile and Proline at the P−1 and P−2 CPD positions. In the crystal structure, the P−1 Leucine side-chain is oriented towards a hydrophobic pocket composed of invariant residues Trp 426, Trp 717, and Thr 386, and the conserved hydrophobic residue Val 384. While less precisely modeled, the main chain position of Leu+2 lies in close proximity to a third hydrophobic pocket composed of the invariant residue Tyr574, and the conserved hydrophobic residues Met 590 and Leu634.

Cdc4 displays little preference for residues in the P+2 to P+5 CPD positions. In the crystal structure, the side chain of P+2 Pro is directed towards solvent (which would account for the lack of selectivity at this position), while the main chain conformation of Pro+1 and Pro+2, causes the CPD to kink away from the peptide-binding surface from the Pro+2 position onwards. As a result, only one additional close contact with Cdc4 is made by the CPD following the Pro+1 position, which consists of a weak hydrogen bond (sub-optimal geometry) between the P+4 Gln side chain and the side chain of Arg 485.

Adjacent to the P+1 proline binding pocket, Ser 464, Thr 441 and Thr 465 are well placed to exert specificity for the +3 and +4 CPD positions if an extended rather than kinked conformation of the CPD were adopted. As noted, Thr 441 and 465 are substituted with Ile in the Cdc4 orthologue pop1 in S. pombe. While nothing is known about the effect of this substitution on CPD recognition, it is predicted that this could have some effect on substrate selectivity for the P+2 to P+5 CPD positions.

Cdc4 displays strong selectivity against Arginine and Lysine in positions −2, −1, +2, +3 and +4. This selectivity may be due to electrostatic repulsion generated by the invariant Cdc4 residues Argininc 572, 534, 467, 485 and 443, which dominate the local electrostatic character of the CPD binding site. Lys 402 is also well placed to contribute to repulsive effects but this position is not conserved amongst the Cdc4 orthologues. The selectivity against positively charged residues in the P−2 to P−1 CPD positions can also be reconciled in part by the hydrophobic nature of the P−1 and P−2 binding pockets and indeed, oppositely charged Glu and Asp residues are also disfavored at these CPD positions.

Comparison with Skp1-Skp2 Complex:

Skp2 is a representative member of a second class of F-box adaptor proteins, which possesses a leucine repeat domain in place of the WD40 repeat of Cdc4. In addition to providing a first structural view of a Skp1 homologue and an F-box domain, the structure of the Skp1/Skp2 complex revealed a mode of molecular association predicted to be employed by all Skp1/F-box homologues. The Cdc4/Skp1/CPD structure confirms the fold of the individual Skp1 and F-box domains and their mode of association. Superposition of yeast and h Skp1 strands β1-β3 and helices α1 to α7 (RMSD Cα=0.74 Å) reveals a close correspondence between F-box helixes α1 to α3 with only Skp1 helix α8 and F-box helix α4 showing significant deviations between the two structures. In addition, only the first half of helix α8 is ordered in ySkp1 and only ha half turn fragment of the F-box helix α4 is apparent in Skp2. The differences in positions and lengths of F-box helices α4 and Skp1 helices α8 reflects the different roles these secondary structure elements play in the linkage between their respective F-box and ligand binding domains.

The structure of the Skp1/Skp2 complex revealed a solid/substantial linkage between its Leucine Repeat and the F-box domains, a feature predicted to be shared by all Skp2 F-box orthologues. In Skp2, the F-box domain helix α4 terminates abruptly and without an appreciable linker, makes an immediate transition to the Leu Repeat domain fold This linkage is enhanced by a β-strand projecting back from C-terminus of the Leu repeat domain and helix α8 projecting forward from Skp1. The sum of linker region interactions compose a local hydrophobic network that links the hydrophobic cores of the F-box domain with that of the LRR domain. This contrast sharply with the corresponding linkage of Cdc4, which is composes primarily by a lengthy inter-domain linker (the helical extension) and which lacks significant involvement of Skp1 or the WD40 repeat domain for stabilization.

Although the Skp2 and Cdc4 F-box adaptor proteins employ structurally divergent ligand binding domains, the general position of the WD40 and LLR domains are surprisingly similar. The precise ligand-binding site on Skp2 has not been determined but mutagenesis sties on the Skp2 orthologue in Met30 have mapped the ligated binding site to the inner side of the curved surface. If the Skp2 binding site is inferred from the overlap with the Cdc4 CPD binding site, the CPD site would map to the lateral side of the Leu repeat domain.

Model of the SCF^{Cdc4} E2 Complex

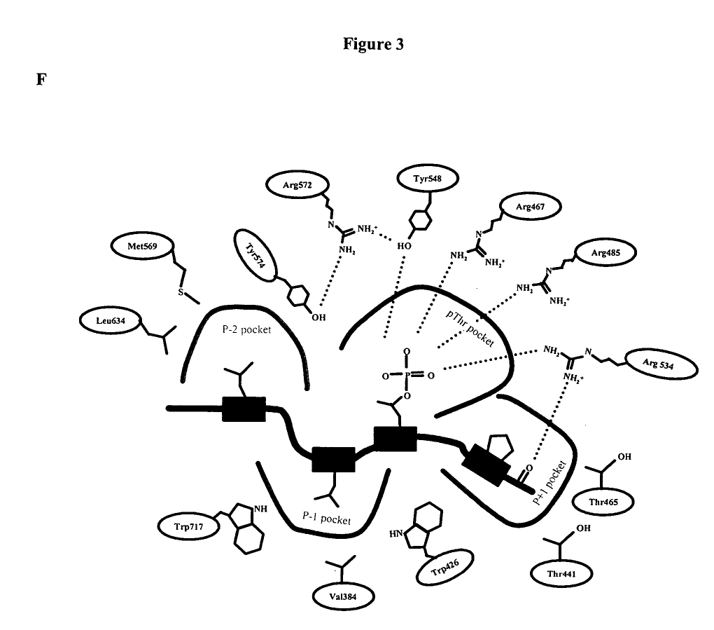

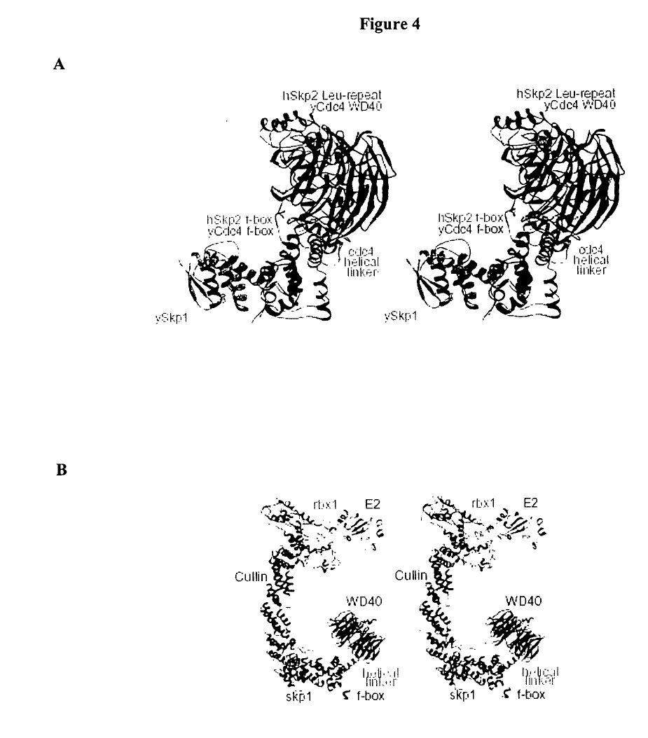

The structure of Cdc4 bound to substrate provides a missing piece of the larger SCF structural puzzle and sheds light on how substrate is presented for ubiqutination. A complete model of the SCF^{Cdc4}-E2-substrate complex consisting of an E2, a cullin, a ring finger domain, an F-box adaptor, Skp1, and CPD has been constructed using the structures of individual component proteins and/or larger assemblies determined previously (FIG. 4). Two interesting features are apparent. Firstly, a separation distance between the E2 active site cysteine and the peptide-binding site of Cdc4 is very large at 64 Å and second Cdc4 presents the CPD peptide with a direct line of sight to the E2.

Mutational Analysis of CPD Binding Surface

In order to probe the functional importance of amino acid residues on the highly conserved peptide binding surface, a panel of Cdc4 mutants (both single and double mutant) were generated and tested each for its ability to bind phospho Sic1 and Skp1 in vitro using a pull down assay and for its ability to substitute for wt-cdc4 in vivo using a cell viability assay. Of 12 single site mutants tested, only Arg 467. Arg485Ala Arg534Ala, and Trp 426 abolished both cell viability in vivo and phosphoSic1 binding in vitro. Together, these residues compose most of the interaction surface with the pThr, Pro CPD core. Interestingly, Tyr 548, the only other amino acid on the surface of CDC4 to directly contact P0 phosphate group, is fractional in vivo but is compromised for CPD binding in vitro. Mutation of the adjacent residue Arg572 to Ala shows the same behavior. For the Arg572 mutation, the inablity to bind psic1 in vitro appears due to its tendency to aggregation. Presumably in the context of the full SCF complex in vivo this mutant is sufficiently well behaved to bind phospho Sic1.

All other single site mutants including Arg443Ala, Lys402Ala, Tyr574Ala, Trp717, Val384, and the double site mutant Thr441/465IIe, K404D/R443D and V384N/W717N are viable when expressed in the cdc4 delete and are fully competent for phosphoSic1 binding in vitro.

Since the cell viability assay nay be masking subtle functional roles for the conserved Cdc4 residues, function was assayed in vivo under more stringent conditions in which Sic1 wt or the stabilized mutants, Sic1 (T33V) or Sic1(T45A) are over-expressed under a galactose promoter. This should amplify defects in cdc4 function. Under these conditions, Trp 717, Tyr 548 and the double mutant K404D/R443D are lethal showing that these residues are in fact important for functions

Role of the Stem and Pedestal Structure

To probe the role of the F-box WD40 inter-domain linker, point mutations, insertions or deletions were introduced into the stem and pedestal structure of Cdc4 and protein function was assessed as performed for the peptide binding site mutants.

Deletion of helix α5 or introduction of Proline and Glycine helix destabilizing residues within the helix had no effect n Cdc4 function both in vitro and in vivo. This result is consistent with the poorly conserved nature f helix α5 and its flanking linker regions. Helix 5 appears entirely absent from human, mouse and drosophila homologues and helix destabilizing substitutions in helix 5 incorporating glycine and proline, are observed in the worm and fungal homologues (FIG. 1). A more invasive deletion of helix 5 that deletes part of the linkers to helix 4 and helix 6 was inviable in yeast. This mutant is properly folded as evidence by the finding that the protein can bind both Skp1 and phospho Sic 1 in vitro. This mutation should likely disrupt the positioning of helix 6 relative to the fbox domain (the linker is too short to span the two secondary structure elements).

The introduction of helix destabilizing residues in helix α6 or the lengthening of the helix by the insertion of one, two, three, four, 8 or 12 amino acid residues also disrupted protein function in vivo, without while maintaining the ability of Cdc4 to to bind pSic1 and Skp1. These results are consistent with a possible role for helix 6 in presenting bound substrates in a specific geometric orientation.

It is peculiar that such a spindly structure is sufficient to maintain rigidity. Perhaps in the context of the dimer, additional contacts help to stabilize position of the WD repeats with respect to the other part of the protein Indeed, the N terminal dimerization domain is required for function. Or perhaps a modicum of flexibility is important for the catalytic mechanism.

Probing Substrate Selectivity Against Positively Charged Residues

Cdc4 bind Sic1 in a multi site dependent manner. Each of the phosphorylation sites in Sic1 are sub-optimal in isolation but series of 5 to 7, they work coopertively to bind to Cdc4 through an avidity effect. Part of the subs optimal character of the sites is due to the presence of Lysine in the +2 to +5 positions. From the crystal structure, the selectivity against positive residues appear to arise from electrostatic replusion from highly conserved residues on the CPD binding surface of Cdc4. To test this hypothesis, two residues not directly involved in phosphopeptide binding were mutated and then the multi site requirement for phosphoSic1 binding was evaluated (FIG. 5c).

Using an IEF pull down assay, wild type Cdc4 is shown to selectively binds to the 5,6 and 7 site phosphorylated phosphoSic1 from a pool of single to 9 site phosphorylated forms. In contrast, the double mutant binds to 3,4,5,6,7 site phosphorylated forms of Sic1 (FIG. 5a). This supports the notion of selectivity and the basis for avidity that may be important for setting sensitive threshold for cell cycle progression (FIG. 5b). The same effect was observed for a double mutant.

Cancer Causing Mutations in Drosophila and Human Cdc4

Mutations in human and fly orthologues of yCdc4 give rise to cancers (see Table below). All mis-sense mutations map to the WD40 CPD binding domain and either have been demonstrated or are predicted to perturb CPD binding function. In previous studies, two cancer cell lines tested positive for mutations at Arginine 534 and Arg 467 (Arg 534 and Arg 467 in yCdc4). In the crystal structure, these residues make a direct binding interaction with the P0 phospho group and our mutational analysis demonstrates an absolute requirement of these residues for CPD binding. In another study, two entrometrial cancerous tissue samples tested positive for mutations equivalent to Arg467 and Arg 485 in yCdc4. As f r the tumor cell line mutations, these mutations affect key residues required for CPD recognition.

Two mutations characterized in drosophila cancer include Ala118Val and Gly1132Glu, corresponding to yCdc4 positions Ser532 and Gly546 respectively. The first of these mutations, involve the substitution of a small Ala/Ser residue with a bulkier b-branched Valine residue. This may compromise CPD binding function through steric effects on the position of Arg434, Arg467, Arg534 triad. In the crystal structure, Ala/Ser is positioned centrally amongst the triad. The second drosophila mutation, Gly1132Glu, maps to β-strand 15 of propeller blade 4 in yCdc4. This position is within the core of the protein and mutation here likely acts by disrupting the overall WD40 domain fold or through local perturbations of structure that indirectly affect the phosphate binding pocket. Glycine in this position of the WD40 repeat motif is highly conserved. The temperature sensitive alleles previously characterized including Gly398Glu in propeller blade 1 and Ser438Asn in propeller blade 2 likely act by disrupting the fold in a similar manner to disrupt the overall WD fold. These are more distantly located from the CPD binding pocket.

Cancer Mutations

| H-cell lines | Drosophila | Entrometrial | Orlicky | Rosamond |
|---|---|---|---|---|
| Arg534(425)Leu | Ser/Ala532(1118)Val | Arg467(465)His | Arg534Ala | Gly398Gln |
| Arg 467(385)Cys | Gly546(1132)Glu | Arg485(479)Gln | Arg467Ala | Ser438Asn |
| | | | Arg485Ala | |
| | | | Trp426Ala | |

Discussion

Recognition of Phosphorylated Substrates by the Ubiquitin System

Substrate selection by Cdc4. The structure of the Skp1-Cdc4-CPD complex reveals the basis for phosphorylation-dependent recognition, the specificity of which is governed by three primary determinants. The substrate phospho-threonine is locked in place by direct contacts with three conserved and essential Arg residues. The preference for hydrophobic residues at the P−1 position (and perhaps P−2 position) is enforced by a hydrophobic pocket that lines the center of the WD40 propeller. Finally, the bias against basic residues at P+2 to P+5 is established by two conserved Arg residues positioned on the top of the propeller directly in-line with the axis of the bound peptide. These conclusions are supported by mutagenesis of key residues in Cdc4 and by structure-based engineering of Cdc4 to accept sub-optimal CPD sequences.

The construction of the Cdc4 phospho-peptide binding module differs from that of known phospho-Ser/Thr binding modules in an important respect. Known phospho-recognition domains, such as 14-3-3, WW and FHA domains appear to be composed of a series of dedicated interaction sites, each of which contributes incrementally to the overall binding interaction (Yaffe and Elia, 2001). The Cdc4-substrate interaction is dominated by extensively coordinated phospho-Thr and Pro residues, as well as by a striking positive electrostatic potential around the binding site. The hydrophobic pocket that selects residues in the P−2 and P−1 positions also contributes to binding affinity. In contrast to other phospho-recognition modules, however, the strong binding of the phosphorylated residue is partially offset by specific selection against basic residues in the substrate peptide, through electrostatic repulsion from a basic patch downstream of the phosphate binding pocket. These features allow the binding affinity for any given peptide to be precisely tuned. Thus, all of the natural CPD motifs in Sic1 are sub-optimal in one or more respects; indeed only peptides derived from the T45 site exhibit any detectable interaction with Cdc4 (Nash et al., 2001). These features establish a requirement for substrate phosphorylation on multiple sites, which mediate a high affinity interaction in a manner that depends cooperatively on the number of phosphorylated residues.

In the case of wild type Sic1, at least six sites must be phosphorylated for high affinity binding by Cdc4. As shown here, mutation of the basic selection residues shifts the binding equilibrium to lower phosphorylated forms while in previous studies, it was demonstrated that introduction of a single optimal CPD into Sic1 causes premature Sic1 degradation and genome stability (Nash et al., 2001). An advantage of this system is that not only can the affinity of individual sites be tuned over a broad range, but the number and spacing of sites can be readily varied to establish a threshold for the targeting kinase. Thus Cdc4 is able to target numerous critical factors for phosphorylation-dependent degradation, including the Cdk inhibitor Sic1, the polarization factor Far1, the replication initiator Cdc6 and the transcription factor Gcn4, all of which may be controlled with different kinetics and different phosphorylation thresholds (Deshaies, 1999). These properties distinguish Cdc4 from other known phospho-peptide binding modules that typically interact with dedicated sites on their substrates through a single high affinity interaction (Pawson and Nash, 2000; Yaffe and Elia, 2001).

The mechanism that engenders a cooperative binding effect remains to be determined. In principle, multiple interactions sites might increase binding either by engaging more than one binding site on Cdc4, or by decreasing the probability of dissociation from Cdc4 (Deshaies and Ferrell, 2001; Harper, 2002; Nash et al., 2001). Cooperative interactions for the dual SH2 domain phosphatase SH-PTP2 and 14-3-3% rely on two substrate binding sites for high affinity recognition of bivalent ligands (Eck et al., 1996; Yaffe et al., 1997). Notably though, inspection of the WD40 surface does not reveal any other potential ligand binding pockets or grooves that might accommodate a phosphorylated peptide motif. Although secondary weak phospho-dependent interactions might occur, it is not obvious from the structure where such putative secondary sites might be located. In favor of the probabilistic cooperativity effect, mathematical modeling suggests that cooperative behaviour arises for the interaction between a single binding site and a polyvalent ligand as a function of the number of ligand sites. In effect multiple ligand sites increase the local concentration of ligand beyond a diffusion limited threshold for escape from the receptor. In the absence of candidate secondary sites, the simplest model is favored in which Cdc4 contains only a single phospho-dependent binding site.

Comparison to other phospho-peptide binding domains. The structure of the Cdc4 WD40 domain provides direct evidence that WD40-type repeats can assemble into propellers with more than seven blades (Fulop and Jones, 1999). One consequence of the additional blade is an enlarged channel through the center of the propeller, which creates a wide binding pocket that accommodates the core Leu-pThr-Pro ligand. This pocket contrasts to all other phospho-Ser/Thr binding domains, which engage their ligand through more shallow surface contacts within loops that extend from the core domain. WD40 domains are known to interact with other proteins in at least two different modes. In the Gb transducin and TUP1 WD40 domains, the protein interaction region occurs across the top of the propeller, much as in the case of Cdc4 (Sprague et al., 2000; Wall et al., 1995). In a second mode, defined for the WD40 domain of clathrin and the b-arrestin peptide, a “peptide-in-groove” interaction occurs on the bottom edge of the propeller between the b-strands of the second blade (ter Haar et al., 2000). Modeling of b-TrCP, which binds the consensus motif DpSGXXpS [SEQ ID NO.42] in IkBa, b-catenin, and Vpu (Yaffe and Elia, 2001), suggests that an extensive basic region on the top of the propeller will engage substrate peptides in an analogous manner to Cdc4.

Spatial orientation of SCF substrates. A conserved feature between all E3 structures solved to date is the large distance between the substrate binding site and the catalytic site (Huang et al., 1999; Zheng et al., 2002; Zheng et al., 2000). Modeling of the Skp1-Cdc4 complex onto a model of the Skp1-Cull-Rbx1-E2 complex suggests that the substrate is positioned for direct frontal attack by the E2 catalytic site but that a gap of some about 65 Å must be bridged between the two sites, presumably by the substrate polypeptide. Unexpectedly, superposition of the WD40 domain of Cdc4 with the LRR of Skp2 does not align the defined phosphopeptide binding pocket of Cdc4 with a potential phospho-recognition site of on the concave face of the LRR repeats (Zheng et al., 2002), at least as defined by mutational analysis of the related F-box protein Grr1 in yeast (Hsiung et al., 2001). If the relative position of substrates in the WD40 versus LRR class of F-box proteins differs, spatial plasticity in substrate presentation must be possible. This notion is consistent with the fact that the HIV protein Vpu is able to redirect the specificity of the F-box protein b-TrCP by bridging bTrCP to the host cell protein CD4, in a manner that depends on phospho-dependent recognition of Vpu by b-TrCP (Margottin et al., 1998). Similarly, it is possible to create synthetic adapters that bridge the substrate recognition site of an F-box protein to an ectopic substrate (Sakamoto et al., 2001). Finally, by definition all E3s must able to accommodate the substrate and the elongating ubiquitin chain generated by repeated catalytic cycles (Pickart, 2001). All of these points argue for considerable spatial leeway, and possibly flexibility of F-box protein orientations within the SCF catalytic cavity.

Based on the extensive Skp1-Skp2 interface, and on the inactivation of Cul1 by insertion of a flexible linker, it has been proposed that SCF complexes, and perhaps E3 enzymes in general, must present substrates to the catalytic site in a rigidly defined fashion (Zheng et al., 2002). However, the WD40 domain and the F-box of Cdc4 are linked only by a single α-helical stalk, with additional surface contact between the domains, all of which is mediated by non-conserved residues. It is thus somewhat difficult to reconcile the properties of the two F-box protein structures solved to date. Although it may be that regions truncated from Cdc4 to enable crystallization may normally help stabilize the interface, none of these regions are highly conserved between closely related Cdc4 family members. Perturbation of the rotational and translational position of the WD40 domain by introduction of additional residues into the stalk abrogates function in all cases, except for a long insertion of 12 residues. The fact that this gross structural change can be tolerated implies a degree of comformational plasticity with the catalytic cradle. This plasticity may facilitate the access of multiple ubiquitination sites within Sic1 to the catalytic center, as directed by the multiple low affinity CPD motifs in Sic1.

Insights into substrate recognition by human Cdc4. In metazoans, Cdc4 targets multiple critical regulators of cell division and development. Among these, cyclin E is a crucial substrate because its abundance must be strictly controlled in order to avoid precocious S phase entry and attendant genome instability (Spruck et al., 1999). Notably, it has been recently reported that mutational inactivation of hCDC4 occurs in several cancer cell lines that exhibit high levels of cyclin E (Moberg et al., 2001; Strohmaier et al., 2001). In addition, hCDC4 may be mutated in up to 30% of endometrial cancers (Spruck et al., 2002). Quite strikingly, known cancer associated mutations in hCDC4 alter phospho Thr-binding residues. Given the probable requirement for homodimerization in active SCF complexes (Kominami et al., 1998; Suzuki et al., 2000), such mutations might be expected to acts in a partial dominant negative manner. Other critical substrates that appear to bind Cdc4 in a phosphorylation dependent manner include SEL-10, a negative regulator of the LIN-12/Notch pathway (Hubbard et al., 1997) that targets the transcriptionally active Notch intracellular domain for degradation (Gupta-Rossi et al., 2002; Wu et al., 2001) and the presenilins, dominant mutations in which predispose to familial early onset Alzheimer's disease (Selkoe, 2001; Wu et al., 1998). Mutations that interfere with hCdc4 activity may therefore compound multiple disease phenotypes.

Yeast and human Cdc4 exhibit a high degree of structural similarity, especially in the critical substrate binding region, and moreover, Cdc4 family members are functionally conserved since the hCdc4 substrate cyclin E is efficiently degraded in yeast in a CDC4-dependent manner (Koepp et al., 2001; Nash et al., 2001; Strohmaier et al., 2001). The structure of yeast Cdc4 thus affords insights for rational drug design. Significantly, the low affinity of individual natural CDP sites that engender the requirement for multisite phosphorylation means that even compounds of moderate affinity can readily out-compete the binding of fully phosphorylated substrates (Nash et al., 2001). Naively, inhibition of hCdc4-substrate interactions would be expected to exacerbate the deregulated proliferation caused by stabilization of cyclinE, Notch-IC or presenilin. However, if Cdc4 or Cdc4-like activities limiting for growth, Cdc4 antagonists may have heightened toxicity in cells that are hypomorphic for Cdc4 function. Alternatively, disruption of hCdc4 function may cause synthetic lethal effects in combination with otherwise non-lethal mutations in functionally overlapping pathways (Tong et al., 2001).

### EXAMPLE 2

The following methods were used in the investigation described in the example: Protein expression and purification. The Cdc4 fragment employed for crystallization was deleted for residues 1-262, 602-605, 609-624, and 745-779 to remove loop regions based on sequence alignments and limited proteolysis of the intact SCF^{Cdc4} complex. Skp1 was deleted for a non-conserved loop insertion spanning residues 37-64. A ^{GST}Skp1-^{His6}Cdc4 complex was co-expressed from plasmid pMT3169 in B934 (DE3) bacterial strain (Stratagene) cells grown in minimal media supplemented with a mixture of selenomethionine (40 μg/ml) and methionine (0.4 ug/ml) and purified by double affinity tag chromatography (Nash et al., 2001). All mutations were constructed by standard methods using oligonucleotides listed in Table 7 and sequence verified in their entirety. Mutants were sub-cloned into pMT3055 or pMT3217 for expression in bacteria or yeast, respectively, as listed in Table 8. The WD40 domain of the helix α6 linker mutants Ala1, Ala2, Ala12, and helix α6 breaker could not be stably expressed in bacteria; the Ala12 mutant also could not be expressed in yeast.

Crystallization, data collection, structure determination and modeling. Hanging drops containing 1 μl of 20 mg/ml protein and 1.2 molar equivalents of the cyclin E derived CPD peptide (acetyl-Gly-Leu-Leu-pThr-Pro-Pro-Gln-Ser-Gly-amide) [SEQ ID NO 40]in buffer (10 mM HEPES pH 7.5, 250 mM NaCl, 1 mM DTT) were mixed with equal volume of reservoir buffer (0.1 M Tris pH 8.5, 1.5 M ammonium sulphate). Crystals of the space group P3₂, (α=107.7 Å, b=107.7 Å, c=168.3 Å, α=γ=90°, β=120°), with two Cdc4-Skp1-CPD complexes in the asymmetric unit were obtained at 20° C. A Multiple Anomalous Dispersion (MAD) experiment was performed on a frozen crystal at the Advanced Photon Source (Argonne, Ill.) beamline BM 14-C and BM 14-D (λ1=0.9798 Å, λ2=0.9800 Å, λ3=0.9000 Å) using a Quantum 4 ADSC CCD detector. Data processing and reduction were carried out with the HKL program suite (Otwinowski and Minor, 1997). The programs SHARP (de La Fortelle and Bricogne, 1997) and SnB (Miller et al., 1994) were used in combination to locate and refine 19 of the total 22 selenium sites. Following phasing and density modification, a model was built using O (Jones et al., 1991) and refined to 2.7 Å resolution with NCS restraints using CNS (Brunger et al., 1998) to a working Rᵥₐₗᵤₑ of 23.8% and R_{free} of 27.3%. Pertinent statistics for data collection and refinement are shown in Table 2. Amino acids 37-74, and 104-115 of Skp1 and amino acids 497-507 of Cdc4 were disordered and could not be modeled. 89.1% of the residues occupy the most favored regions of the Ramachandran plot, 10.8% the additional allowed region and 0.2% the generously allowed region.

Ribbons representations were generated using Ribbons (Carson, 1991), surface representations were generated using Grasp (Nicholls et al., 1991) and electron density maps were generated using O (Jones et al., 1991). A model of the ubiquitin-E2-SCF^{Cdc4}-CPD complex was generated by superposition of the Skp1 subunits of the Skp1-Cdc4-CPD structure and the Skp1-Cul1-Rbx1 structure (PDB ID 1LDK) (Zheng et al., 2002), the RING finger domains from Rbx1 in the same Skp1-Cul1-Rbx1 complex and from the Cb1 subunit of the Cb1-UbCH7 structure (PDB ID 1FBV) (Zheng et al., 2000), and the E2 subunits of the Cb1-UbCH7 structure and an NMR-based Ubc1-ubiquitin model (PDB ID 1FXT) (Hamilton et al., 2001). The Skp1, RING domain and E2 subunits overlapped with RMSD values of 1.01 Å, 2.09 Å, and 2.04 Å respectively.

Cdc4 functional assays. CDC4 mutant alleles were assessed for complementation of a cdc4A strain in a plasmid shuffle assay (Nash et al., 2001). Sensitivity to SIC1 dosage was determined by transformation with pMT837 (GAL1-SIC1) or pMT767 (GAL1-SIC1^{T33V}) and plating on glucose medium or galactose medium. For in vitro capture of phospho-Sic1 by Cdc4, 0.5 μg of bacterially-expressed ^{HIS6}Sic1 was phosphorylated with immobilized Cln2-Cdc28 kinase from baculovirus-infected Sf9 cells and then incubated with 1 μg of immobilized wild type or mutant Cdc4²⁶³⁻⁷⁴⁴-GST-Skp1, at 4° C. for 1 hr, washed 4 times and visualized by anti-Sic1 immunoblot. For isoelectric focusing (IEF)-2D gel analyses, an evenly distributed pool of phospho-Sic1 isoforms was generated by combining different time points in a Sic1 phosphorylation reaction. 2.5 μg of the phospho-Sic1 pool was bound to 5 μg of immobilized wild type or mutant Cdc4¹⁻⁷⁴⁴-GST-Skp1. Captured isoforms were separated by denaturing IEF-2D gel electrophoresis using pH3-10NL Immobiline gel strips (Amersham) and visualized by anti-Sic1 immunoblot. Alternatively, the pool of phospho-Sic1 isoforms was incubated in solution with a ubiquitination reaction mix containing ATP, ubiquitin, yeast E1, Cdc34 and either wild type or mutant SCF^{Cdc4} complex, composed of a 1:1 ratio of bacterial Cdc4-GST-Skp1 and insect cell-produced Cdc53-Rbx1, at 30° C. for 1 h as previously described (Nash et al., 2001).

Results

Alignment of Skp1 and Cdc4 homologs from various species and limited proteolysis of full length recombinant proteins were used to deduce loop regions in Saccharomyces cerevisiae Skp1 and Cdc4 that might interfere with protein crystallization (FIG. 1). Crystals of a ternary complex of ScSkp1 bound to ScCdc4 and a CPD phosphopeptide were obtained that diffracted to a resolution of 2.7 Å (Table 2). For Skp1, a non-essential loop spanning residues 37-64 was removed. The Cdc4 fragment used extends from residues 263 to 744, which encompasses the F-box motif to the end of the WD40 domain, and was engineered to remove two predicted loop regions (FIG. 1B). This Cdc4 construct lacks an essential ˜40 reside domain that precedes the F-box in different WD40 domain containing F-box protein family embers (Wolf et al, 1999). The high affinity CPD phosphopeptide corresponds to nine residues of human cyclin E, Gly-Leu-Leu-pThr-Pro-Pro-Gln-Ser-Gly, [SEQ ID NO. 40] which binds Cdc4 with a K_{d} of 1 μM (Nash et al., 2001).

The F-box interface. Yeast Skp1 forms an elongated structure with a mixed α/β topology identical to that reported for human Skp1 (Schulman et al., 2000) and consists of a three-strand β sheet, denoted β1 to β3, and eight α-helices, denoted α1 to α8 (FIG. 2A). The structure of Cdc4 consists of an F-box domain, an α-helical linker, and a WD40 domain (FIG. 2A,B,C). The F-box domain is comprised of five α helices, denoted α0 to α4. This topology differs slightly from that reported for the F-box domain of hSkp2, which consists of a loop region L1 and three helices denoted 1 to α3 (Schulman et al., 2000) Helix α0 in Cdc4 corresponds most closely in sequence and position to the loop region L1 of hSkp2 while a half turn ant of Cdc4 helix α4 is discernable in the transition sequence between the hSkp2 F-box and the LRR domain. As observed in the hSkp1-hSkp2 complex, ScSkp1 and the F-box domain of Cdc4 associate by interdigitation of helixes α0 to α3 Cdc4 with helices α5 to α8 of Skp1, with the interface itself comprised of an inter-protein 4-helix bundle. This mode of association gives rise to a contiguous hydrophobic core that spans Skp1 and the F-box domain of Cdc4. Superposition of the yeast and human structures reveals that Skp1 helix α8 and F-box helix α4 deviate significantly in that only the first half of helix α8 is ordered in ScSkp1 and only a half turn fragment of the F-box helix α4 is apparent in hSkp2 (FIG. 6A). The difference in position and length of F-box helix α4 and Skp1 helix α8 reflects the different roles these secondary structure elements play in the linkage between their respective F-box and ligand binding domains, as described below.

The WD40 domain. Eight copies of the WD40 repeat motif in Cdc4 form an 8 blade β-propeller structure (FIG. 6B). The WD40 repeat motif of approximately 40 residues composes the outer β-strand of one propeller blade and the inner three stands of the adjacent blade in a continuous circular arrangement (Fulop and Jones, 1999). The actual Cdc4 structure contrasts to the 7 blade β-propeller predicted for Cdc4 and its orthologs based on previously solved WD40 domain structures, all of which contain only 7 blades (Koepp et al., 2001; Nash et al., 2001). This discrepancy is attributable to the cryptic nature of the 8th WD40 repeat motif. Structure based sequence alignment suggests that the WD40 domains of the F-box proteins Met30 and β-TRCP will form canonical 7-blade β-propeller structures (FIG. 1B). A variant five β-strand structure occurs in blade 2, in which a large insert in tie β12-β13 linker allows the outermost β9¹ strand to run parallel to the β9 strand. This five strand composition is unique to the fungal Cdc4 orthologs. In terms of overall structural dimensions, the WD40 domain resembles a conical frustum of 40 Å diameter top surface and 50 Å bottom surface, an overall thickness of 30 Å and a central pore of 6 Å diameter. The CPD binding site resides on the top surface of the frustum and runs across the edge of the pore, while the bottom surface of the frustum links to the F-box domain.

The F-box to WD40 domain linker. The F-box domain of Cdc4 is followed by a helical extension that forms a structured bridge to the WD40 domain. The bridge consists of two α-helices, α5 and α6, that together with helices α3 and α4 of the F-box domain form a platform and stalk-like structure that positions the WD40 domain well away from the F-box domain (FIG. 2A,C). The relative orientation of the F-box domain and WD40 domain is imposed almost entirely through the integrity of the stalk-like helix α6, which is 30 Å in length. The N-terminal end of helix α6 is anchored into the hydrophobic core of the F-box domain through interactions involving α6 residues Phe 355 and Leu 356 and F-box residues Ile 295, and Ile 296, Leu 315, Trp 316, and Leu 319 (FIG. 2C). Helix α5 packs along side the base of helix α6 opposite to the F-box domain through hydrophobic interactions involving Tyr342, Leu 338 and Leu 334. The C-terminal end of helix α6 inserts obliquely between propeller blades α7 and α8 of the WD40 domain through van der Wals and hydrophobic interactions involving residues Trp 365 and Ile 361 with WD40 domain residues Val 687, Ile 696, Leu 726 and Phe 743 in propeller blades 7 and 8. Asn 364 of helix α6 also forms a tight hydrogen bond with the backbone carbonyl group of Phe 743 in propeller blade 8. The conservation of many of these residues, with the possible exception of those within helix α5, suggests that a structured linkage between the WD40 and F-box domains may be a common feature of the WD40 family F-box proteins.

The interdomain connection between the F-box and the WD40 domains of Cdc4 appears less rigidly structured than the corresponding region in hSkp2 (FIG. 6A). Outside of the stalk helix α6, only two close contacts (<3.5 Å) are observed between the WD40 domain and other regions of Cdc4 (FIG. 2C). These contacts consist of hydrogen bonds between Asn684 and Arg700 in two loop regions of propeller blade 7 with Glu 323 in the α4-α5 linker of the helical extension. Both hydrogen bonds are maintained in the two Cdc4 molecules of the crystal asymmetric unit but all three residues are poorly conserved amongst Cdc4 orthologues (FIG. 1B). The lack of additional stabilizing interactions suggests that the F-box to WD40 domain linker is not exceedingly rigid, and indeed, the WD40 domain in the two Cdc4 molecules of the crystal asymmetric unit differ relative to their F-box domains by a 5° rotation about the long axis of helix α6. In contrast, in hSkp2 the F-box domain helix α4 terminates abruptly in an immediate transition to the LRR domain fold such that the adjoined domains form a rigid hydrophobic core (Schulman et al., 2000). Although the Skp2 and Cdc4 families of F-box proteins employ structurally divergent F-box interfaces, the general position of the WD40 and LLR domains are nonetheless similar (FIG. 6A).

Model of the SCFCd^{Cdc4}E2 complex. The structure of the Skp1-Cdc4-CPD complex sheds light on how substrates are presented by the F-box protein to the E2 for ubiquitin transfer. A complete model of the E2-SCF^{Cdc4}-substrate complex consisting of ubiquitin, hUbc7, hCul1, hRbx1, ScCdc4, ScSkp1, and the CPD peptide is shown in FIG. 6B. This model is based on the reconstructed E2-SCF^{Skp2} complex derived by Pavletich and colleagues (Zheng et al., 2002), in conjunction with an NMR-based ubiquitin-E2 thioester model (Hamilton et al., 2001). Two interesting features are apparent. First, the distance between the E2 active site cysteine and the phosphate group of the bound CPD peptide is approximately 59 Å, which is similar to the spacing reported between the substrate interaction site and the E3 catalytic site in the hUbc7-Cb1 structure (Zheng et al., 2000). Secondly, the WD40 domain presents the CPD peptide in a direct line-of-sight to the E2. Although the ligand-binding site on hSkp2 has not been determined, mutagenesis studies on the LRR-containing F-box protein Grr1 in yeast suggest that substrates bind to the inner side of the curved repeat surface (Hsiung et al., 2001). If the position of this site is maintained in hSkp2, then the LRR domain of Skp2 is predicted to project substrates in an orthogonal direction to that of the Cdc4 WD40 domain (FIG. 6A).

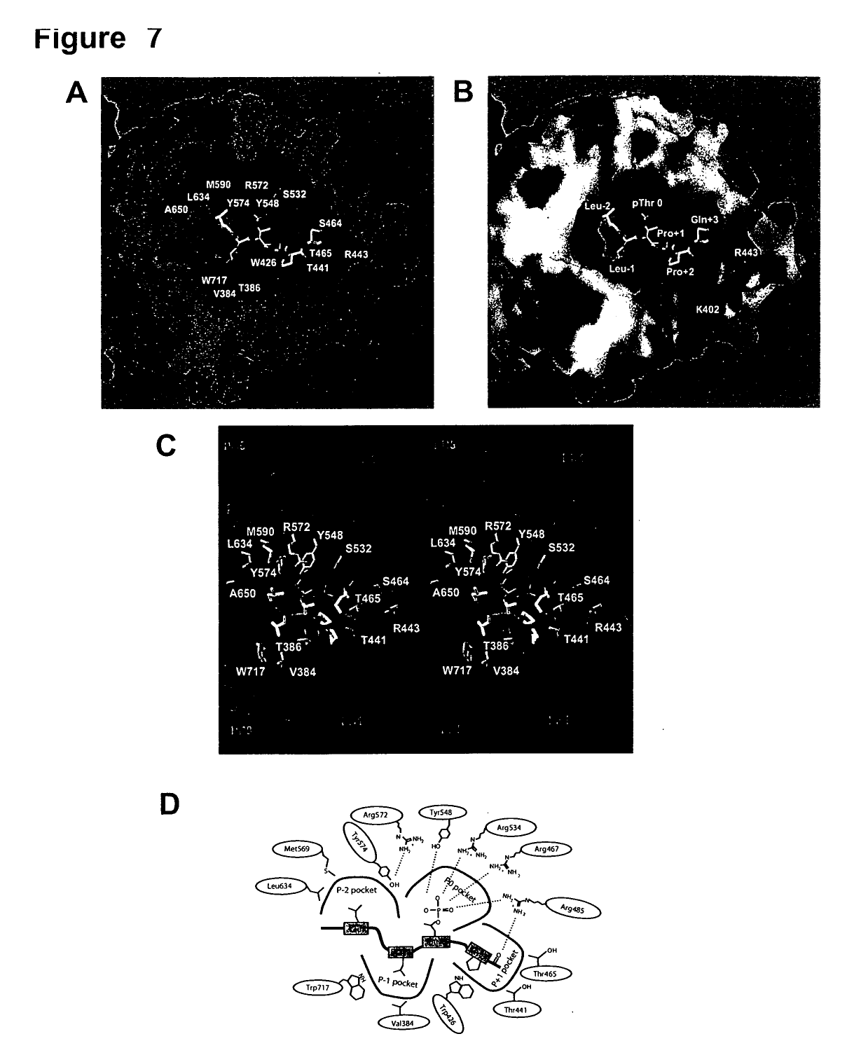

Phosphopeptide recognition. The CPD binding surface represents the most conserved part of the WD40 repeat domain structure (FIG. 7A-D). The central CPD sequence Leu-pThr-Pro-Pro [SEQ ID NO. 41] was modeled unambiguously in unbiased experimental electron density maps in both Skp1-Cdc4-CPD complexes of the crystal asymmetric unit (FIG. 3). Interpretable electron density is also apparent for the P−2 Leu, P+3 Gln, P+4 Ser, and P+5 Gly positions, but only in one complex of the crystal asymmetric unit. The CPD peptide binds in an extended manner across β-propeller blade 2 with the N-terminus oriented towards the central pore of the WD40 domain and the C-terminus oriented towards the outer rim. Identical substrate peptide orientations and contacts were observed for an independent Skp1-Cdc4-CPD structure with a phosphopeptide derived from the transcription factor Gcn4, which is a physiological substrate of Cdc4 in yeast (Meimoun et al., 2000; Chi et al., 2001). However, of the Gcn4 peptide sequence, Phe-Leu-Pro-pThr-Pro-Val-Leu-Glu-Asp [SEQ ID NO. 43], only the core residues Pro-pThr-Pro had discernable electron density.

The CPD sequence requirements for the CPD-Cdc4 interaction are fully accounted for by structural elements in the WD40 domain. An absolute requirement for phosphorylation at Ser or Thr at the P−0 position of the CPD derives from a network of electrostatic interactions and hydrogen bonds that coordinate the P0 pThr phosphate group (FIG. 7C, D). This interaction is mediated by residues that are conserved across all Cdc4 orthologs (FIG. 1B). The P0 phosphate group forms direct electrostatic interactions with the guanidinium groups of Arg485, Arg467, and Arg534 and a direct hydrogen bond with the side chain of Tyr548. The side chain of Tyr548 is coordinated by stacking interactions with the guanidinium group of Arg572, which in turn is coordinated by a hydrogen bond to the side chain of Tyr574. Although Cdc4 shows a 6-fold preference for pThr over pSer (Nash et al., 2001), the structural basis for this selectivity is not obvious since the Cy methyl group of Thr is directed towards solvent and does not make contact with the WD40 domain surface.

A second absolute requirement for CPD-Cdc4 interaction rests on the P+1 proline, the side chain of which projects into a three-sided pocket on the WD40 surface. One side of this pocket is formed by the side chain of Trp 426, which packs in a coplanar manner with the P+1 proline side chain. The opposite side of this binding pocket is formed by the side chain of Arg 485 via coordination of the proline side chain and backbone carbonyl group through van der Waals and hydrogen bonding interactions, respectively. The side chains of Thr 441 and Thr 465 define the remaining side of the P+1 proline binding pocket, with Cy side chain groups composing a hydrophobic surface. The hydroxyl groups of Thr 441 and 465 orient away from the P+1 binding pocket, where they are well placed to influence binding specificity for CPD residues C-terminal to the P+1 position. Unlike Trp 426 and Arg 485, which are invariant amongst the Cdc4 orthologs, Thr 441 and Thr 465 are both substituted with lie in the S. pombe Cdc4 ortholog Pop1 (FIG. 1B). This substitution might restrict CPD sequences able to bind Pop1 through steric or hydrophobic constraints on residues C-terminal to the P+1 proline position.

Cdc4 displays a strong preference for the hydrophobic residues Leu/Ile/Pro at the P−1 and Leu/Ile at the P−2 CPD positions. In the crystal structure, the P−1 Leucine side-chain fits into a hydrophobic pocket composed of invariant residues Trp 426, Trp 717, and Thr 386, and the conserved hydrophobic residue Val 384. While less precisely modeled, the main chain position of Leu−2 lies in close proximity to a third hydrophobic pocket composed of the invariant residue Tyr574, and the conserved hydrophobic residues Met 590 and Leu634. The hydrophobic character of the P−1 and P−2 pockets is manifest as selection against both charged and small polar residues at these positions in the CPD consensus (Nash et al., 2001).

The WD40 phospho-recognition domain of Cdc4 is unusual in that it exhibits strong selectivity against either Arg or Lys residues in the P+2 to P+5 CPD positions, but otherwise shows no sequence preference at these positions (Nash et al., 2001). In the crystal structure, the side chain of P+2 Pro is directed towards solvent, while the main chain conformation of Pro+1 and Pro+2 causes the CPD to kink away from the peptide-binding surface from the Pro+2 position onward. As a result, only one additional contact with Cdc4 is made by the CPD following the Pro+1 position, namely a weak hydrogen bond with sub-optimal geometry between the P+4 Gin side chain and the side chain of Arg 485. Because the P+1 Pro main chain is forced away from the WD40 domain surface, the selection against basic residues in the P+2, +3, +4 and +5 positions in the CPD consensus is almost certainly due to through-space electrostatic repulsion. This effect arises from a dominant positive electrostatic potential generated by both the invariant triad of Arg residues that comprise the core pThr-Pro binding pocket, and by a radial extension of the surface due to Arg 572, Arg 443 and Lys 402, the former two of which are conserved amongst Cdc4 orthologs (FIG. 7B).

A number of natural mutations detected in metazoan orthologs of Cdc4 corroborate the structure-based analysis. Two ovarian cancer cell lines bear missense mutations at conserved Arg residues that correspond to Arg 467 and Arg 534 in yeast Cdc4 (Moberg et al., 2001). In the crystal structure, these residues make direct contact with the P0 phosphate group and are essential for function (FIG. 7C, D). In a recent study of human primary endometrial tumors, mutations in phosphate-binding Arg residues equivalent to Arg 467 and Arg 485 were detected in 2 of 13 tumor samples (Spruck et al., 2002). Other cancer-associated nonsense and frameshift mutations truncate hCdc4 within the WD40 domain (Moberg et al., 2001; Strohmaier et al., 2001; Spruck et al., 2002). Similarly, all three characterized mutations in the Drosophila ago gene that lead to excess cell proliferation affect the WD40 domain (Moberg et al., 2001). One of these mutations, Ala1118Val, corresponding to position SerS32 in ScCdc4 substitutes a conserved small residue with a bulkier residue at the center of the critical Arg 434-Arg467-Arg534 triad (FIG. 7C).

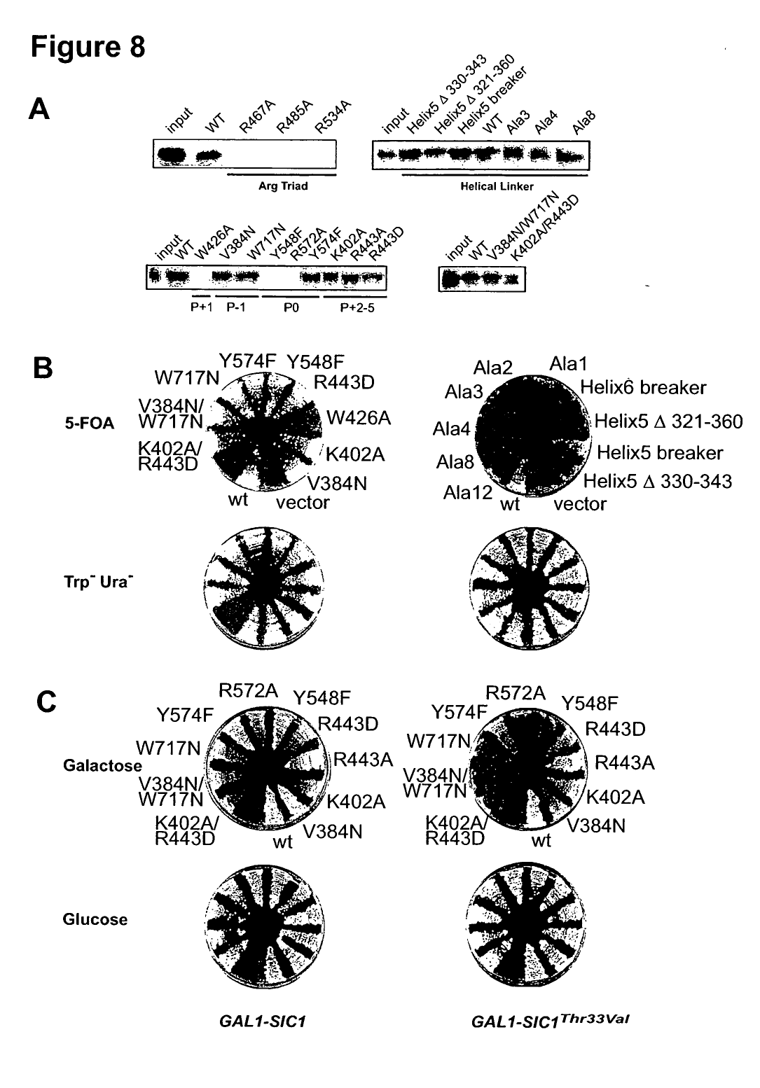

Mutational analysis of the F-box to WD40 domain linker. To probe the importance of orientation and rigidity in the F-box WD40 inter-domain linker, point mutations, insertions or deletions were introduced into the platform and stalk structure of Cdc4. None of these deletions affected the ability of the recombinant proteins to bind phospho-Sic1 in vitro or protein abundance in vivo (FIG. 8A and data not shown). Introduction of the helix destabilizing residues glycine and proline into helix α5 did not compromise Cdc4 function in vivo (FIG. 8B), consistent with the poorly conserved nature of this region (FIG. 1B). However, two different deletions of helix α5 eliminated Cdc4 function in vivo, indicating that the F-box-WD40 domain interface is an essential structural component. Similarly, placement of helix destabilizing residues at the center of helix α6 or the lengthening of this helix by the insertion of one, two, three, four, 8 or 12 amino acid residues disrupted Cdc4 function in vivo. Helix α6 is thus critical for productive orientation of the WD40 domain.

Mutational analysis of the CPD binding surface. Previous mutational analysis based on sequence conservation in the Cdc4 family identified Arg467, Arg485 and Arg534 as essential for substrate binding and function in yeast (Nash et al., 2001). Two of the three corresponding residues in hCdc4, Arg 417 and Arg 457, are essential for the binding of phospho-cyclin E, while the third corresponding to Arg485 was not tested (Koepp et al., 2001). To systematically probe the role of residues that form the highly conserved peptide binding surface, a panel of Cdc4 mutants was generated and each were tested for pSic1 binding in vitro, complementation of a cdc4A strain and sensitivity to increased SIC1 dosage. Four mutants, Arg467Ala, Arg485Ala, Arg534Ala, and Trp426Ala were unable to bind phospho-Sic1 in vitro or complement a cdc4A strain, but were fully competent for Skp1 binding (FIG. 8A, B). The essential function of these residues is not confined to elimination of Sic1 because none of the corresponding mutant alleles were able to rescue a cdc4Δ sic1Δ strain. These results reflect the critical structural role played by these residues in coordination of the P0 phosphate and the P+1 proline of the CPD. Mutation of the remaining phosphate-coordinating residue, Tyr548, did not cause loss of viability but did result in dosage sensitivity to SIC^{Thr}33Val, which encodes a partially stabilized version of Sic1 (FIG. 8C). Mutation of Arg 572 had the same effect, as befits the observed stacking interaction between this residue and Tyr 548. Although both mutants were severely impaired for binding to phospho-Sic1 in vitro, this effect may be exacerbated by the tendency of these recombinant proteins to aggregate. In summary, the six residues that directly or indirectly coordinate the primary pThr-Pro core motif are critical for CPD recognition in vitro and Cdc4 function in vivo.

Disruption of residues that confer selection at the P−2, P−1 and P+2 to P+5 positions had only modest effects on the ability of Cdc4 to target pSic1. A Trp717Asn mutation predicted to disrupt the P−1 pocket conferred sensitivity to dosage of SIC1^{Thr33Val}, but did not overtly affect the pSic1-Cdc4 interaction in vitro. Individual mutations in all other residues that are well positioned to affect substrate selection, namely Arg443Ala, Arg443Asp, Lys402Ala, Tyr574Phe and Val384Asn were indistinguishable from wild type in each of the assays used. Substrate selection residues on the WD40 surface thus contribute only modestly if at all to the essential function of Cdc4. As described below, however, these residues play a subtle but critical role in setting the phosphorylation threshold for the CPD-Cdc4 interaction.

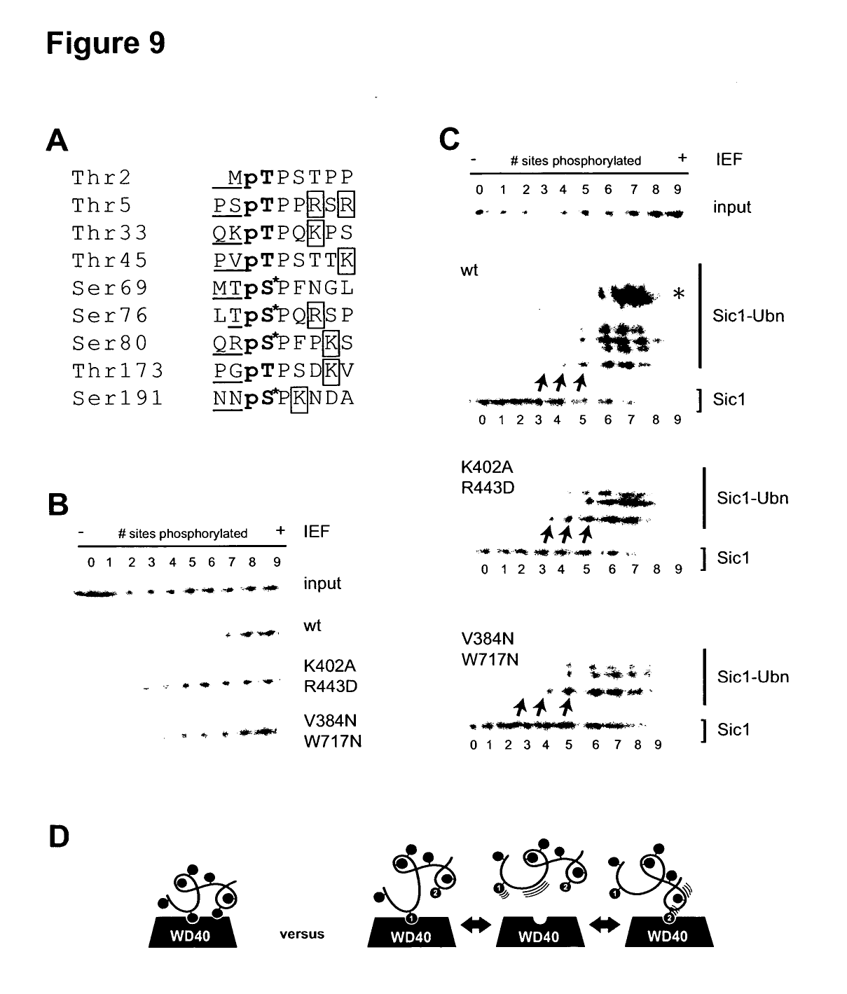

Modulation of CPD substrate selectivity. A critical feature of the Sic1-Cdc4 interaction is the requirement for phosphorylation of Sic1 on multiple sites. To enforce this requirement, each of the phosphorylation sites in the native Sic1 sequence are sub-optimal in one or more respects (FIG. 9A). The Cdc4-CPD structure suggests that selectivity against basic residues may be due to electrostatic repulsion generated from the conserved patch of basic residues in and around the CPD binding pocket, while selection for hydrophobic residues arises from the P−1 pocket that is composed in part by Val 384 and Trp717. To examine the basis for selection against sub-optimal CPD motifs, the effects of mutations in non-essential residues in these two regions on the multisite phosphorylation requirement for Sic1 recognition were assessed.

The ability of Cdc4 to capture various phosphoisoforms of wild type Sic1 from a pool of recombinant Sic1 that had been phosphorylated to various extents by Cln2-Cdc28 was monitored. As resolved by isoelectric focusing, this pool contained roughly equal amounts of Sic1 phosphorylated on 1, 2, 3, 4, 5, 6, 7, 8 and 9 sites. Wild type Cdc4 was only able to capture Sic1 phosphorylated on six or more sites (FIG. 9B). This result formally demonstrates the transition in binding affinity between 5 and 6 phosphorylation sites, as initially inferred from capture of a series of Sic1 phosphorylation site mutants by Cdc4 (Nash et al., 2001). The role of positive electrostatic potential in selecting against sub-optimal CPD sequences with basic residues at C-terminal positions was tested with the Lys402Ala Arg443Asp double mutant. This mutant was able to select Sic1 phosphoisoforms that contained as few as three phosphorylation sites (FIG. 9B). The ability of the Lys402Ala Arg443Asp double mutant to capture lower phosphorylated forms of Sic1 is also evident in one-dimensional SDS-PAGE (FIG. 8A). Similarly, perturbation of the P−1 hydrophobic pocket with a Val384Asn Trp717Asn double mutation allowed capture of Sic1 phosphorylated on as few as four sites. These in vitro binding results were recapitulated in solution-based in vitro ubiquitination assays with wild type and mutant forms of Cdc4. Both double mutant forms of Cdc4 were able to convert Sic1 phosphoryated on four or five sites to oligo-ubiquitinated species, whereas wild type Cdc4 was unable to do so (FIG. 9C). The double mutants were, however, less efficient than wild type at elaborating fully ubiquitinated species of phospho-Sic1, perhaps because of protein stability effects or interference with catalytic steps after substrate binding. This interpretation is consistent with the sensitivity of strains bearing the double mutant alleles to SIC1^{Thr33Val} dosage (FIG. 8B). Overall, re-engineering of negative selection residues in the Cdc4 WD40 domain supports the notion that the series of sub-optimal CPD motifs in Sic1 sets a high phosphorylation threshold for its recognition by Cdc4.

Discussion

The structure of the Skp1-Cdc4-CPD complex provides direct visualization of substrate orientation within an SCF complex. Insights gained from the structure include the unexpectedly frail interface between the F-box and the WD40 repeat domain, the basis for dedicated pThr-Pro dipeptide recognition by a novel eight-blade WD40 propeller, and a detailed understanding of the basis for selection against natural CPD sequences. The latter feature appears to be tailored to enforce multisite phosphorylation dependent degradation of Sic1, which in turn would help engender a highly cooperative onset of DNA replication (Nash et al., 2001). Similar principles may well operate for other Cdc4 substrates, including cyclin E, Notch^{IC} and presenilin in mammalian cells (Strohmaier et al., 2001; Lai, 2002; Selkoe, 2001). Because yeast and human Cdc4 are structurally and functionally analogous (Nash et al., 2001; Strohmaier et al., 2001; Koepp et al., 2001), the structure of yeast Cdc4 affords obvious insights for pharmacological modulation of hCdc4 function in these pathways. Interestingly, a significant proportion of characterized human and fly CDC4 mutations alter residues in the CPD binding pocket. Given the probable requirement for homodimerization in active SCF complexes (Wolf et al., 1999), such mutations might act in a partial dominant negative manner to confer a growth advantage in the heterozygous state.

Phospho-recognition by Cdc4. The specificity of phosphorylation-dependent recognition by the WD40 domain of Cdc4 is governed by three main determinants: (i) a dedicated pThr-Pro binding pocket; (ii) a deep hydrophobic pocket that selects hydrophobic residues N-terminal to the phosphorylation site, and (iii) a through space electrostatic selection against basic residues C-terminal to the phosphorylation site. As for all documented phospho-dependent lipid/protein recognition modules, the Cdc4 WD40 domain employs arginine residues to directly contact the phosphate group of the ligand. However, unlike most domains in which adjacent residues impose subtle effects on specificity (Yaffe and Elia, 2001), the P+1 proline is an integral component of the core binding determinant (Nash et al., 2001). In the Cdc4-CPD co-crystal, ligand residues are locked in place by direct contact of the phosphate and proline carbonyl groups with three conserved and essential Arg residues, while the proline side chain inserts into a tight hydrophobic pocket formed by Trp426, Thr441, and Thr465. Because the phospho-binding pocket infrastructure has no obvious demarcation between the pThr and Pro binding sites, the Cdc4 WD40 domain is in effect a dedicated pThr-Pro binding module.

Comparison to other peptide recognition modules. Interesting parallels can be drawn between the Cdc4 WD40 domain, 14-3-3 domains and the class IV WW domains, which all have the ability to recognize phospho-Ser/Thr epitopes in the context of adjacent proline residues (Yaffe and Elia, 2001). The interaction of the Pin1 class IV WW domain with a pSer-Pro peptide differs from Cdc4 in that it does not rely on an extensive network of Arg residues for phosphate coordination (Verdecia et al., 2000). However, a striking similarity between Pin1 and Cdc4 lies in the P+1 proline binding pocket, which in both cases depend on a highly conserved tryptophan side chain to engage the P+1 proline pyrrolidine ring through a coplanar interaction. In contrast to Cdc4, Pin1 actually displays a preference for Arg in the P+2 position, such that the binding specificity of the pSer-Pro recognition domain closely matches that of the targeting CDK enzymes.

14-3-3 domains bind pSer epitopes with a preference, but not an absolute requirement, for proline residues at the P+2 position (Yaffe et al., 1997). This less stringent selection arises because the 14-3-3 proline binding pocket is able to accommodate other residues with propensity to form β-turns. Interestingly, the proline preferences in both the 14-3-3 and Cdc4 WD40 domains give rise to the same qualitative effect: in each case the prolines terminate direct interactions between the peptide and the ligand binding domain by orienting the peptide away from the domain surface. In the case of Cdc4, biologically significant electrostatic effects operate in spite of the loss of direct peptide contact. Physiologically relevant substrate anti-selection mediated by charge repulsion is unique amongst known protein interaction modules.

The structure of the Cdc4 WD40 domain provides direct evidence that WD40-type repeats can assemble into propellers with more than seven blades (Fulop and Jones, 1999). WD40 domains are known to interact with other proteins in at least two different modes, either across the front face of the propeller, as in the case of Cdc4, or on the outer edge of the propeller as in the case of clathrin (ter Haar et al., 2000). Modeling of the F-box protein P-TrCP, which binds the doubly phosphorylated consensus motif DpSGXXpS [SEQ ID NO. 42] in IκβBβ-catenin, and Vpu (Yaffe and Elia, 2001), reveals an extensive conserved basic region on the front face of the propeller, which may engage substrate phosphoepitopes in an analogous manner to Cdc4.

Spatial orientation of SCF substrates. A conserved feature between all E3 structures solved to date is the substantial distance between the substrate binding site and the catalytic site (Huang et al., 1999; Zheng et al., 2000; Zheng et al., 2002). Superposition of the Skp1-Cdc4 complex onto a model of the Skp1-Cull-Rbx1-E2-ubiquitin complex suggests that the substrate is positioned for direct frontal attack by the E2 catalytic site, but that a gap of some 59 Å between the two sites must be bridged, presumably by the substrate polypeptide. The disordered structure of Sic1 lends itself to this possibility (Nash et al., 2001). Intriguingly, overlay of the WD40 domain of Cdc4 with the LRR of Skp2 does not align the defined phosphopeptide binding pocket of Cdc4 with a potential phospho-recognition site on the concave face of the LRR repeats (Zheng et al., 2002), at least as defined by mutational analysis of the related F-box protein Grr1 in yeast (Hsiung et al., 2001). If the relative position of substrates in the WD40 versus LRR class of F-box proteins do in fact differ, spatial leeway in substrate presentation must be possible.

Based on the extensive Skp1-Skp2 interface, and on the inactivation of Cull by insertion of a flexible linker, it has been proposed that SCF complexes, and perhaps E3 enzymes in general, must present substrates to the catalytic site in a rigidly defined fashion (Zheng et al., 2002). Unexpectedly, the WD40 domain and the F-box of Cdc4 are linked only by a single α-helical stalk, with very limited additional contacts. Despite the lack of sequence conservation in the a helix 6 structure that supports the WD40 domain, spatial constraints are nevertheless evident, as shown by the sensitivity of the structure to rotational and translational shifts caused by insertion of additional residues into the stalk. It is also possible that regions truncated from Cdc4 to enable crystallization may normally help stabilize the inter-domain interface.

Cooperativity in substrate selection by Cdc4. The properties of the Cdc4 phosphopeptide binding module differ from those of other known modules in the important respect that the interaction with core recognition elements is partially offset by specific selection against basic residues in the substrate peptide. This feature establishes an intrinsic antagonism between the recognition mechanism and the targeting CDK kinases, which prefer Ser/Thr-Pro sites with C-terminal basic residues (Endicott et al., 1999). Significantly, all of the natural CPD motifs in Sic1 contain one or more mismatches to the optimal CPD consensus. This system based on positive and negative ligand selection may not only set an elevated threshold for kinase activity, but may also allow the threshold to be precisely tuned for any given substrate by varying the number, spacing and properties of each site. Thus, Cdc4 is able to target numerous critical factors for phosphorylation-dependent degradation, including the Cdk inhibitor Sic1, the CDK inhibitor and polarization factor Far1, the replication initiator Cdc6 and the transcription factor Gcn4, all of which may be controlled with different kinetics and different phosphorylation thresholds (Patton et al., 1998). In one extreme, typified by Gcn4 and cyclin E, the substrate may contain a high affinity site that is augmented by several minor low affinity sites (Meimoun et al., 2000; Chi et al., 2001; Strohmaier et al., 2001). In the other, more akin to Sic1, a large number of weak sites may cooperate to drive high affinity binding only when a phosphorylation threshold is reached. As shown here, mutation of either the distal basic selection region or the P−1 pocket in Cdc4 shifts the binding equilibrium to lower phosphorylated forms of Sic1, which, in the absence of other structural effects that may compromise Cdc4, would be predicted to cause premature DNA replication and genome stability (Nash et al., 2001). These features distinguish Cdc4 from other known phospho-peptide binding modules characterized to date that typically interact with dedicated sites on their substrates through a single high affinity interaction.

The mechanism that underlies the cooperative binding transition of the phospho-Sic1-Cdc4 interaction between five and six phosphorylation sites remains to be determined. In principle, multiple interactions sites might increase binding by engaging more than one binding site on Cdc4 (FIG. 9D). This type of cooperative interaction is common in biological systems, as in the avidity of antibodies for polyvalent ligands and pathogen-host interactions (Mammen et al., 1998). Analogous cooperative binding interactions occur in signaling pathways. For instance, the dual SH2 domain phosphatase SH-PTP2 and the 14-3-3ζ protein both engage two substrate binding sites on their respective ligands (Eck et al., 1996; Yaffe et al., 1997). However, inspection of the Cdc4 WD40 domain surface does not reveal any obvious ligand binding sites that might accommodate a second phosphorylated peptide motif, nor is there any biochemical evidence for secondary binding sites (Nash et al., 2001). In addition, the wide range of substrates and site spacing accommodated by Cdc4, including random concatamers of synthetic CPD sites (Nash et al., 2001), is a priori difficult to explain by two or more fixed binding sites on Cdc4.

Instead, a model is favored that requires only a single phospho-dependent binding site on Cdc4 (FIG. 9D). In this scheme, phosphorylation of multiple CPD sites on Sic1 increases the local concentration of sites around Cdc4 once the first CPD site is bound, to the point where diffusion limited escape from the receptor is overwhelmed by the probability of re-binding of any one CPD site. In a sense, Sic1 becomes kinetically trapped in close proximity to Cdc4. Mathematical modeling of an idealized polyvalent ligand-monovalent receptor interaction indicates that the rate of ligand escape from the receptor exhibits a negative exponential dependence on the number of ligand sites. The term allovalent is proposed to describe the ability of multiple weak spatially separated ligand sites to cooperatively interact with a single receptor site. The prevalence of multisite phosphorylation (Cohen, 2000), and indeed of polyvalent ligands in general (Mammen et al., 1998), suggests that this type of behavior may underlie many biological processes.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims. In particular it will be appreciated that the references to specific amino acid residues for particular a SCF complexes, and components thereof (e.g. F-box protein) illustrated in the Tables and Figures, in no way limits the scope of the invention and it will be appreciated that a person skilled in the art could determine the specific corresponding amino acid residues for other SCF complexes and components thereof.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Data Collection, Structure Determination, and Refinement
Statistics

| | Peak | Inflection | Remote |
|---|---|---|---|
| Wavelength (Å) | 0.9798 | 0.9800 | 0.9000 |
| Resolution (Å) | 2.8 | 2.9 | 2.7 |
| R_{sym} (%) | 5.9 (38.7) | 6.1 (36.1) | 5.0 (28.9) |
| Total Reflections | 311509 | 187010 | 298371 |
| Unique Reflections | 107167 | 96027 | 116218 |
| Completeness (%) | 99.8 (99.1) | 99.3 (98.3) | 97.7 (93.6) |
| I/σ | 9.9 (2.7) | 7.4 (2.1) | 10.1 (2.9) |
| Phasing Power | | | |
| Refinement statistics | | | |
| Resolution range (Å) | 20-2.8 | | |
| Reflections | 103863 | | |
| R_{factor}/R_{free} (%) | 24.09/28.71 | | |
| Rms deviations | | | |
| Bonds (Å) | 0.0091 | | |
| Angles (°) | 1.3453 | | |

Space group P3₂: a = b = 107.7 Å, c = 168.3 Å; a = b = 90°, c = 120°;
Two molecules per asymmetric unit.
¹Data for the highest resolution shell (2.90-2.80 Å)
²Rsym = 100 × Σ|I − <I>|/Σ<I>, where I is the observed intensity and <I> is the average intensity from multiple observations of symmetry-related reflections.
³Phasing power for isomorphous and anomalous acentric reflections, where phasing power = <(|F_{h,c}|/phase-integrated lack of closure)>.
⁴R_{free} was calculated with 8.8% of the data.

TABLE 2
Data Collection, Structure Determination, and Refinement
Statistics

| | Peak | Inflection | Remote |
|---|---|---|---|
| Phasing Statistics | | | |
| Wavelength (Å) | 0.9798 | 0.9800 | 0.9000 |
| Resolution (Å) | 2.8 (2.9-2.8) | 2.9 (3.0-2.9) | 2.7 (2.8-2.7) |
| R_{sym} (%) | 5.9 (37.2) | 6.1 (36.1) | 5.0 (28.9) |
| Total Reflections | 311509 | 187010 | 298371 |
| Unique Reflections | 107167 | 96027 | 116218 |
| Completeness (%) | 99.8 (99.1) | 99.3 (98.3) | 97.7 (93.6) |
| I/σ | 9.9 (2.7) | 7.4 (2.1) | 10.1 (2.9) |
| Phasing Power | 5.2/1.3 | 4.0/0.94 | 0/0.91 |
| (ISO/ANO) | | | |
| Refinement statistics | | | |
| (remote wavelength) | | | |
| Resolution range (Å) | 20-2.7 | # protein | 9364 |
| | | atoms | |
| Reflections | 113960 | # water | 72 |
| | | molecules | |
| R_{factor}/R_{free} (%) | 23.8/27.3 | | |
| Rms deviations | | | |
| Bonds (Å) | 0.0089 | | |
| Angles (°) | 1.42 | | |

Space group P3₂: a = b = 107.7 Å, c = 168.3 Å; a = b = 90°, c = 120°
Two Skp1-Cdc4-CPD complexes per asymmetric unit
Numbers given in parantheses refer to data for the highest resolution shell.
¹Rsym = 100 × Σ|I − <I>|/Σ<I>, where I is the observed intensity and <I> is the average intensity from multiple observations of symmetry-related reflections.
²Phasing power for isomorphous and anomalous acentric reflections, = <[|Fₕ(calc)/phase-integrated lack of closure]>.
³R_{free} was calculated with 8.8% of the data.

TABLE 3
Atomic Contacts of a Substrate Binding Pocket

| No. of | | | |
|---|---|---|---|
| Atomic | CDC4 WD40 Motif | CDC4 atomic | CPD Motif |
| Interaction | Atomic Contact | Contact | Atomic Contact |
| 1 | Ile 295 | Phe 255 | |
| | Ile296 | Leu356 | |
| | Leu 315 | | |
| | Trp 316 Leu 319 | | |
| 2 | Val 687 | Trp 365 | |
| | Ile 696 | Ile 364 | |
| | Leu 726 | | |
| | Phe 743 | | |
| 3 | Phe 743 | Asn 364 | |
| 4 | Asn 684 | Glu 323 | |
| | Arg 700 | | |
| 5 | Arg 485 | | PO pTyr |
| | Arg 467 | | Phosphate |
| | Arg 534 | | |
| | Tyr 548 | | |
| 6 | Trp 426 | | P + 1 Proline side |
| | Arg 485 | | chain |
| | Thr 386 | | |
| | Thr 441 | | |
| | Thr 465 | | |
| 7 | Trp 426 | | P + 1 Leucine side |
| | Trp 717 | | chain |
| | Thr 386 | | |
| | Val 384 | | |
| 8 | Tyr 574 | | Leucine + 2 |
| | Thr 386 | | |
| | Val 384 | | |

TABLE 4
Atomic Contacts of a Substrate Binding Pocket

| No. of | CDC4 WD40 | | | Atomic |
|---|---|---|---|---|
| Atomic | Motif/F-box Domain | CDC4 atomic | CPD Motif | Interaction |
| Interaction | Atomic Contact | Contact | Atomic Contact | Property |
| 1 | Ile 295 | Phe 355 | | hydrophobic |
| | Ile296 | Leu356 | | interactions and |
| | Leu 315 | | | van der Wals |
| | Trp 316 | | | interactions |
| | Leu 319 | | | |
| 2 | Val 687 | Trp 365 | | van der Wals and |
| | Ile 696 | Ile 361 | | hydrophobic |
| | Leu 726 | | | interactions |
| | Phe 743 | | | |
| 3 | Phe 743 | Asn 364 | | hydrogen bond |
| 4 | Asn 684 | Glu 323 | | hydrogen bonds |
| | Arg 700 | | | |
| 5 | Arg 485 | | PO pTyr or pSer | electrostatic |
| | Arg 467 | | Phosphate at P-O | interactions |
| | Arg 534 | | position of CPD | hydrogen bond |
| | Tyr 548 | | | |
| 6 | Trp 426 | | P + 1 Proline side | hydrogen and van |
| | Arg 485 | | chain and | der Wals |
| | Thr 441 | | backbone carbonyl | hydrophobic |
| | Thr 465 | | of CPD | interations |
| 7 | Trp 426 | | P − 1 Leucine (or | hydrophobic |
| | Trp 717 | | Ile/Pro) side chain | interactions |
| | Thr 386 | | | |
| | Val 384 | | | |
| 8 | Tyr 574 | | Leucine − 2 | hydrophobic |
| | Met 590 | | Leu/Ile at P − 2 | interactions |
| | Leu 634 | | position | |
| 9. | Tyr 342 | | | hydrophobic |
| | Leu 338 | | | interactions |
| | Leu 334 | | | |

TABLE 5
Determinants of pThr-Pro binding

| | P-Sic1 | gal | gal | gal | | | | | | |
|---|---|---|---|---|---|---|---|---|---|---|
| | pull down | Sic1 wt | Sic1T45A | Sic1T33V | gal Sic1-7 | gal Sic1-6 | gal Sic1-5 | gal Sic1-4 | gal Sic1-3 | shuffle |
| WT | bind | alive | dead | alive | | | | | | alive |
| [symbol] | [symbol] | | | | | | | | | [symbol] |
| R443A | bind | alive | dead | alive | | | | | | alive |
| [symbol] | [symbol] | | | | | | | | | [symbol] |
| R467A | no bind | | | | | | | | | dead |
| R485A | no bind | | | | | | | | | dead |
| R534A | no bind | | | | | | | | | dead |
| R572A | no bind | alive | dead | dead | | | | | | alive |
| [symbol] | [symbol] | | | | | | | | | alive |
| W426A | no bind | | | | | | | | | dead |
| V384N | bind | alive | dead | alive | | | | | | alive |
| K402A | bind | alive | dead | alive | | | | | | alive |
| R443D | bind | alive | dead | alive | | | | | | alive |
| Y548F | no bind | alive | dead | dead | | | | | | alive |
| Y574F | bind | alive | dead | alive | | | | | | alive |
| W717N | bind | alive | dead | dead | | | | | | alive |
| T441I/T465I | (in progress) | | | | | | | | | (in progress) |
| K402A/R443D | bind | alive | dead | dead | | | | | | alive |
| W717N/V384N | bind | alive | dead | dead | | | | | | alive |
| quad | (in progress) | | | | | | | | | dead |

TABLE 6
REMARK peptide link removed (applied DPEP) : from A 31 to A 45
REMARK peptide link removed (applied DPEP) : from A 73 to A 86
REMARK peptide link removed (applied DPEP) : from B 496 to B 508
REMARK peptide link removed (applied DPEP) : from C 31 to C 45
REMARK peptide link removed (applied DPEP) : from C 73 to C 86
REMARK peptide link removed (applied DPEP) : from D 496 to D 508
REMARK peptide link removed (applied DPPP) : from E 4 to E 5
REMARK coordinates from minimization and B-factor refinement
REMARK refinement resolution: 20 - 2.8 A
REMARK starting r= 0.2415 free_r= 0.2846
REMARK final r= 0.2409 free_r= 0.2871
REMARK rmsd bonds= 0.009114 rmsd angles= 1.34531
REMARK B rmsd for bonded mainchain atoms= 1.230 target= 1.5
REMARK B rmsd for bonded sidechain atoms= 1.778 target= 2.0
REMARK B rmsd for angle mainchain atoms= 2.103 target= 2.0
REMARK B rmsd for angle sidechain atoms= 2.675 target= 2.5
REMARK target= mlf final wa= 2.77695
REMARK final rweight= 0.1078 (with wa= 2.77695)
REMARK md-method= torsion annealing schedule= constant
REMARK starting temperature= 2000 total md steps= 1 * 100
REMARK cycles= 2 coordinate steps= 20 B-factor steps= 10
REMARK sg= P3(2) a= 107.669 b= 107.669 c= 168.3 alpha= 90 beta= 90 gamma= 120
REMARK topology file 1 : CNS_TOPPAR:protein.top
REMARK topology file 2 : CNS_TOPPAR:dna-rna.top
REMARK topology file 3 : CNS_TOPPAR:water.top
REMARK topology file 4 : CNS_TOPPAR:ion.top
REMARK topology file 5 : CNS_TOPPAR:tpo.top
REMARK parameter file 1 : CNS_TOPPAR:protein_rep.param
REMARK parameter file 2 : CNS_TOPPAR:dna-rna_rep.param
REMARK parameter file 3 : CNS_TOPPAR:water_rep.param
REMARK parameter file 4 : CNS_TOPPAR:ion.param
REMARK parameter file 5 : CNS_TOPPAR:tpo.param
REMARK molecular structure file: automatic
REMARK input coordinates: 36modl.pdb
REMARK reflection file= remote.cv
REMARK ncs= none
REMARK B-correction resolution: 6.0 - 2.8
REMARK initial B-factor correction applied to fobs :
REMARK B11= 1.580 B22= 1.580 B33= −3.160
REMARK B12= −3.767 B13= 0.000 B23= 0.000
REMARK B-factor correction applied to coordinate array B: 0.915
REMARK bulk solvent: density level= 0.324998 e/A\hat{ }3, B-factor= 34.4718 A+E,+CIR 2
REMARK reflections with |Fobs|/sigma_F < 0.0 rejected
REMARK reflections with |Fobs|> 10000 * rms(Fobs) rejected
REMARK anomalous diffraction data was input
REMARK theoretical total number of refl. in resol. range: 107240 (100.0%)
REMARK number of unobserved reflections (no entry or |F|=0): 3377 (3.1%)
REMARK number of reflections rejected: 0 (0.0%)
REMARK total number of reflections used: 103863 (96.9%)
REMARK number of reflections in working set: 93784 (87.5%)
REMARK number of reflections in test set: 10079 (9.4%)
CRYST1 107.669 107.669 168.300 90.00 90.00 120.00 P 32
REMARK FILENAME=“ref37.pdb”
REMARK DATE:28-Jun-2002 13:23:24 created by user: orlicky
REMARK VERSION:1.1

| | | | | | | | | | | |
|---|---|---|---|---|---|---|---|---|---|---|
| ATOM | 1 | CB | SER A | 2 | 72.279 | 75.039 | 74.638 | 1.00 | 40.45 | A |
| ATOM | 2 | OG | SER A | 2 | 72.875 | 75.230 | 73.368 | 1.00 | 36.62 | A |
| ATOM | 3 | C | SER A | 2 | 70.142 | 75.473 | 73.446 | 1.00 | 40.01 | A |
| ATOM | 4 | O | SER A | 2 | 69.547 | 74.397 | 73.338 | 1.00 | 39.92 | A |
| ATOM | 5 | N | SER A | 2 | 70.277 | 75.520 | 76.026 | 1.00 | 40.09 | A |
| ATOM | 6 | CA | SER A | 2 | 70.953 | 75.797 | 74.713 | 1.00 | 40.80 | A |
| ATOM | 7 | N | ASN A | 3 | 70.145 | 76.398 | 72.482 | 1.00 | 38.91 | A |
| ATOM | 8 | CA | ASN A | 3 | 69.428 | 76.196 | 71.221 | 1.00 | 37.67 | A |
| ATOM | 9 | CB | ASN A | 3 | 68.480 | 77.367 | 70.926 | 1.00 | 38.01 | A |
| ATOM | 10 | CG | ASN A | 3 | 67.193 | 77.305 | 71.736 | 1.00 | 38.13 | A |
| ATOM | 11 | OD1 | ASN A | 3 | 66.616 | 76.236 | 71.944 | 1.00 | 38.39 | A |
| ATOM | 12 | ND2 | ASN A | 3 | 66.733 | 78.458 | 72.184 | 1.00 | 36.20 | A |
| ATOM | 13 | C | ASN A | 3 | 70.310 | 75.996 | 69.990 | 1.00 | 35.89 | A |
| ATOM | 14 | O | ASN A | 3 | 71.503 | 76.275 | 69.995 | 1.00 | 34.48 | A |
| ATOM | 15 | N | VAL A | 4 | 69.685 | 75.500 | 68.928 | 1.00 | 36.46 | A |
| ATOM | 16 | CA | VAL A | 4 | 70.343 | 75.275 | 67.639 | 1.00 | 33.96 | A |
| ATOM | 17 | CB | VAL A | 4 | 70.545 | 73.750 | 67.383 | 1.00 | 33.36 | A |
| ATOM | 18 | CG1 | VAL A | 4 | 70.533 | 73.449 | 65.916 | 1.00 | 33.70 | A |
| ATOM | 19 | CG2 | VAL A | 4 | 71.855 | 73.295 | 67.985 | 1.00 | 34.50 | A |
| ATOM | 20 | C | VAL A | 4 | 69.415 | 75.889 | 66.584 | 1.00 | 31.88 | A |
| ATOM | 21 | O | VAL A | 4 | 68.209 | 76.036 | 66.818 | 1.00 | 31.04 | A |
| ATOM | 22 | N | VAL A | 5 | 69.972 | 76.282 | 65.443 | 1.00 | 31.25 | A |
| ATOM | 23 | CA | VAL A | 5 | 69.146 | 76.853 | 64.376 | 1.00 | 31.10 | A |
| ATOM | 24 | CB | VAL A | 5 | 69.458 | 78.346 | 64.086 | 1.00 | 31.85 | A |
| ATOM | 25 | CG1 | VAL A | 5 | 68.586 | 78.825 | 62.927 | 1.00 | 32.81 | A |
| ATOM | 26 | CG2 | VAL A | 5 | 69.188 | 79.192 | 65.314 | 1.00 | 31.47 | A |
| ATOM | 27 | C | VAL A | 5 | 69.339 | 76.075 | 63.089 | 1.00 | 29.35 | A |
| ATOM | 28 | O | VAL A | 5 | 70.448 | 75.952 | 62.588 | 1.00 | 29.57 | A |
| ATOM | 29 | N | LEU A | 6 | 68.232 | 75.561 | 62.574 | 1.00 | 28.63 | A |
| ATOM | 30 | CA | LEU A | 6 | 68.206 | 74.777 | 61.355 | 1.00 | 28.72 | A |
| ATOM | 31 | CB | LEU A | 6 | 67.299 | 73.558 | 61.559 | 1.00 | 27.60 | A |
| ATOM | 32 | CG | LEU A | 6 | 67.585 | 72.619 | 62.739 | 1.00 | 25.63 | A |
| ATOM | 33 | CD1 | LEU A | 6 | 66.684 | 71.402 | 62.592 | 1.00 | 24.10 | A |
| ATOM | 34 | CD2 | LEU A | 6 | 69.043 | 72.205 | 62.781 | 1.00 | 21.89 | A |
| ATOM | 35 | C | LEU A | 6 | 67.667 | 75.632 | 60.208 | 1.00 | 28.54 | A |
| ATOM | 36 | O | LEU A | 6 | 66.577 | 76.200 | 60.316 | 1.00 | 28.48 | A |
| ATOM | 37 | N | VAL A | 7 | 68.416 | 75.740 | 59.112 | 1.00 | 27.74 | A |
| ATOM | 38 | CA | VAL A | 7 | 67.945 | 76.548 | 57.978 | 1.00 | 25.90 | A |
| ATOM | 39 | CB | VAL A | 7 | 69.092 | 77.413 | 57.366 | 1.00 | 25.69 | A |
| ATOM | 40 | CG1 | VAL A | 7 | 68.515 | 78.450 | 56.403 | 1.00 | 25.12 | A |
| ATOM | 41 | CG2 | VAL A | 7 | 69.889 | 78.077 | 58.450 | 1.00 | 22.70 | A |
| ATOM | 42 | C | VAL A | 7 | 67.374 | 75.650 | 56.869 | 1.00 | 24.30 | A |
| ATOM | 43 | O | VAL A | 7 | 68.069 | 74.768 | 56.337 | 1.00 | 21.91 | A |
| ATOM | 44 | N | SER A | 8 | 66.111 | 75.881 | 56.522 | 1.00 | 23.21 | A |
| ATOM | 45 | CA | SER A | 8 | 65.453 | 75.091 | 55.486 | 1.00 | 23.15 | A |
| ATOM | 46 | CB | SER A | 8 | 63.966 | 75.433 | 55.375 | 1.00 | 21.22 | A |
| ATOM | 47 | OG | SER A | 8 | 63.794 | 76.735 | 54.826 | 1.00 | 20.44 | A |
| ATOM | 48 | C | SER A | 8 | 66.093 | 75.428 | 54.167 | 1.00 | 25.17 | A |
| ATOM | 49 | O | SER A | 8 | 66.851 | 76.389 | 54.054 | 1.00 | 27.53 | A |
| ATOM | 50 | N | GLY A | 9 | 65.782 | 74.635 | 53.155 | 1.00 | 26.78 | A |
| ATOM | 51 | CA | GLY A | 9 | 66.329 | 74.914 | 51.847 | 1.00 | 26.99 | A |
| ATOM | 52 | C | GLY A | 9 | 65.726 | 76.208 | 51.320 | 1.00 | 27.95 | A |
| ATOM | 53 | O | GLY A | 9 | 66.120 | 76.682 | 50.256 | 1.00 | 29.65 | A |
| ATOM | 54 | N | GLU A | 10 | 64.762 | 76.782 | 52.039 | 1.00 | 26.66 | A |
| ATOM | 55 | CA | GLU A | 10 | 64.160 | 78.025 | 51.582 | 1.00 | 25.22 | A |
| ATOM | 56 | CB | GLU A | 10 | 62.637 | 77.927 | 51.610 | 1.00 | 24.78 | A |
| ATOM | 57 | CG | GLU A | 10 | 62.045 | 76.721 | 50.905 | 1.00 | 24.26 | A |
| ATOM | 58 | CD | GLU A | 10 | 60.543 | 76.567 | 51.203 | 1.00 | 27.17 | A |
| ATOM | 59 | OE1 | GLU A | 10 | 59.747 | 77.407 | 50.727 | 1.00 | 27.67 | A |
| ATOM | 60 | OE2 | GLU A | 10 | 60.144 | 75.617 | 51.929 | 1.00 | 28.38 | A |
| ATOM | 61 | C | GLU A | 10 | 64.598 | 79.236 | 52.416 | 1.00 | 25.50 | A |
| ATOM | 62 | O | GLU A | 10 | 63.853 | 80.213 | 52.538 | 1.00 | 24.72 | A |
| ATOM | 63 | N | GLY A | 11 | 65.794 | 79.164 | 52.995 | 1.00 | 25.06 | A |
| ATOM | 64 | CA | GLY A | 11 | 66.299 | 80.264 | 53.792 | 1.00 | 25.04 | A |
| ATOM | 65 | C | GLY A | 11 | 65.740 | 80.473 | 55.192 | 1.00 | 25.64 | A |
| ATOM | 66 | O | GLY A | 11 | 66.399 | 81.110 | 56.016 | 1.00 | 25.60 | A |
| ATOM | 67 | N | GLU A | 12 | 64.552 | 79.946 | 55.483 | 1.00 | 26.56 | A |
| ATOM | 68 | CA | GLU A | 12 | 63.945 | 80.113 | 56.806 | 1.00 | 27.47 | A |
| ATOM | 69 | CB | GLU A | 12 | 62.510 | 79.614 | 56.782 | 1.00 | 26.92 | A |
| ATOM | 70 | CG | GLU A | 12 | 61.661 | 80.451 | 55.874 | 1.00 | 32.01 | A |
| ATOM | 71 | CD | GLU A | 12 | 60.215 | 80.003 | 55.841 | 1.00 | 35.92 | A |
| ATOM | 72 | OE1 | GLU A | 12 | 59.912 | 78.942 | 55.244 | 1.00 | 37.74 | A |
| ATOM | 73 | OE2 | GLU A | 12 | 59.367 | 80.716 | 56.419 | 1.00 | 39.01 | A |
| ATOM | 74 | C | GLU A | 12 | 64.705 | 79.459 | 57.951 | 1.00 | 28.74 | A |
| ATOM | 75 | O | GLU A | 12 | 65.222 | 78.345 | 57.826 | 1.00 | 29.61 | A |
| ATOM | 76 | N | ARG A | 13 | 64.804 | 80.170 | 59.069 | 1.00 | 29.55 | A |
| ATOM | 77 | CA | ARG A | 13 | 65.513 | 79.615 | 60.207 | 1.00 | 30.68 | A |
| ATOM | 78 | CB | ARG A | 13 | 66.457 | 80.651 | 60.832 | 1.00 | 32.30 | A |
| ATOM | 79 | CG | ARG A | 13 | 66.654 | 81.907 | 60.002 | 1.00 | 34.52 | A |
| ATOM | 80 | CD | ARG A | 13 | 67.459 | 82.959 | 60.756 | 1.00 | 35.08 | A |
| ATOM | 81 | NE | ARG A | 13 | 68.816 | 82.514 | 61.051 | 1.00 | 36.91 | A |
| ATOM | 82 | CZ | ARG A | 13 | 69.454 | 82.778 | 62.188 | 1.00 | 37.09 | A |
| ATOM | 83 | NH1 | ARG A | 13 | 68.846 | 83.485 | 63.129 | 1.00 | 35.87 | A |
| ATOM | 84 | NH2 | ARG A | 13 | 70.691 | 82.328 | 62.391 | 1.00 | 37.00 | A |
| ATOM | 85 | C | ARG A | 13 | 64.511 | 79.130 | 61.238 | 1.00 | 29.61 | A |
| ATOM | 86 | O | ARG A | 13 | 63.494 | 79.783 | 61.506 | 1.00 | 29.30 | A |
| ATOM | 87 | N | PHE A | 14 | 64.809 | 77.962 | 61.789 | 1.00 | 28.74 | A |
| ATOM | 88 | CA | PHE A | 14 | 63.980 | 77.337 | 62.797 | 1.00 | 28.90 | A |
| ATOM | 89 | CB | PHE A | 14 | 63.507 | 75.945 | 62.356 | 1.00 | 27.81 | A |
| ATOM | 90 | CG | PHE A | 14 | 62.614 | 75.946 | 61.147 | 1.00 | 26.25 | A |
| ATOM | 91 | CD1 | PHE A | 14 | 63.149 | 75.925 | 59.867 | 1.00 | 25.67 | A |
| ATOM | 92 | CD2 | PHE A | 14 | 61.235 | 75.955 | 61.291 | 1.00 | 27.94 | A |
| ATOM | 93 | CE1 | PHE A | 14 | 62.324 | 75.912 | 58.745 | 1.00 | 25.90 | A |
| ATOM | 94 | CE2 | PHE A | 14 | 60.393 | 75.942 | 60.174 | 1.00 | 27.89 | A |
| ATOM | 95 | CZ | PHE A | 14 | 60.940 | 75.921 | 58.896 | 1.00 | 26.23 | A |
| ATOM | 96 | C | PHE A | 14 | 64.847 | 77.185 | 64.032 | 1.00 | 30.32 | A |
| ATOM | 97 | O | PHE A | 14 | 66.004 | 76.760 | 63.959 | 1.00 | 30.71 | A |
| ATOM | 98 | N | THR A | 15 | 64.305 | 77.558 | 65.176 | 1.00 | 32.94 | A |
| ATOM | 99 | CA | THR A | 15 | 65.067 | 77.412 | 66.396 | 1.00 | 34.55 | A |
| ATOM | 100 | CB | THR A | 15 | 64.910 | 78.650 | 67.275 | 1.00 | 34.81 | A |
| ATOM | 101 | OG1 | THR A | 15 | 65.362 | 79.797 | 66.548 | 1.00 | 35.82 | A |
| ATOM | 102 | CG2 | THR A | 15 | 65.737 | 78.509 | 68.541 | 1.00 | 36.02 | A |
| ATOM | 103 | C | THR A | 15 | 64.535 | 76.179 | 67.119 | 1.00 | 34.29 | A |
| ATOM | 104 | O | THR A | 15 | 63.358 | 75.830 | 66.984 | 1.00 | 35.01 | A |
| ATOM | 105 | N | VAL A | 16 | 65.398 | 75.501 | 67.859 | 1.00 | 34.16 | A |
| ATOM | 106 | CA | VAL A | 16 | 64.954 | 74.330 | 68.592 | 1.00 | 36.47 | A |
| ATOM | 107 | CB | VAL A | 16 | 64.597 | 73.166 | 67.628 | 1.00 | 38.19 | A |
| ATOM | 108 | CG1 | VAL A | 16 | 65.697 | 72.981 | 66.579 | 1.00 | 37.94 | A |
| ATOM | 109 | CG2 | VAL A | 16 | 64.403 | 71.884 | 68.421 | 1.00 | 40.71 | A |
| ATOM | 110 | C | VAL A | 16 | 65.992 | 73.862 | 69.601 | 1.00 | 36.55 | A |
| ATOM | 111 | O | VAL A | 16 | 67.199 | 74.020 | 69.398 | 1.00 | 36.18 | A |
| ATOM | 112 | N | ASP A | 17 | 65.511 | 73.294 | 70.699 | 1.00 | 36.09 | A |
| ATOM | 113 | CA | ASP A | 17 | 66.398 | 72.817 | 71.750 | 1.00 | 36.61 | A |
| ATOM | 114 | CB | ASP A | 17 | 65.586 | 72.082 | 72.812 | 1.00 | 38.44 | A |
| ATOM | 115 | CG | ASP A | 17 | 66.458 | 71.416 | 73.840 | 1.00 | 40.95 | A |
| ATOM | 116 | OD1 | ASP A | 17 | 66.418 | 70.164 | 73.924 | 1.00 | 43.10 | A |
| ATOM | 117 | OD2 | ASP A | 17 | 67.184 | 72.137 | 74.556 | 1.00 | 41.69 | A |
| ATOM | 118 | C | ASP A | 17 | 67.499 | 71.902 | 71.196 | 1.00 | 35.77 | A |
| ATOM | 119 | O | ASP A | 17 | 67.218 | 70.903 | 70.543 | 1.00 | 33.05 | A |
| ATOM | 120 | N | LYS A | 18 | 68.757 | 72.245 | 71.471 | 1.00 | 36.17 | A |
| ATOM | 121 | CA | LYS A | 18 | 69.897 | 71.473 | 70.972 | 1.00 | 35.91 | A |
| ATOM | 122 | CB | LYS A | 18 | 71.208 | 72.003 | 71.541 | 1.00 | 35.90 | A |
| ATOM | 123 | CG | LYS A | 18 | 72.397 | 71.086 | 71.239 | 1.00 | 36.70 | A |
| ATOM | 124 | CD | LYS A | 18 | 73.679 | 71.525 | 71.964 | 1.00 | 37.63 | A |
| ATOM | 125 | CE | LYS A | 18 | 74.131 | 72.918 | 71.523 | 1.00 | 38.26 | A |
| ATOM | 126 | NZ | LYS A | 18 | 75.528 | 73.223 | 71.956 | 1.00 | 38.28 | A |
| ATOM | 127 | C | LYS A | 18 | 69.776 | 70.010 | 71.319 | 1.00 | 36.47 | A |
| ATOM | 128 | O | LYS A | 18 | 70.048 | 69.129 | 70.497 | 1.00 | 35.04 | A |
| ATOM | 129 | N | LYS A | 19 | 69.388 | 69.756 | 72.559 | 1.00 | 38.15 | A |
| ATOM | 130 | CA | LYS A | 19 | 69.220 | 68.393 | 73.011 | 1.00 | 38.79 | A |
| ATOM | 131 | CB | LYS A | 19 | 68.733 | 68.374 | 74.456 | 1.00 | 39.52 | A |
| ATOM | 132 | CG | LYS A | 19 | 68.597 | 66.980 | 75.018 | 0.00 | 40.07 | A |
| ATOM | 133 | CD | LYS A | 19 | 68.074 | 66.996 | 76.439 | 0.00 | 40.81 | A |
| ATOM | 134 | CE | LYS A | 19 | 66.638 | 67.489 | 76.505 | 0.00 | 41.30 | A |
| ATOM | 135 | NZ | LYS A | 19 | 66.086 | 67.373 | 77.885 | 0.00 | 41.65 | A |
| ATOM | 136 | C | LYS A | 19 | 68.202 | 67.734 | 72.084 | 1.00 | 38.59 | A |
| ATOM | 137 | O | LYS A | 19 | 68.508 | 66.734 | 71.445 | 1.00 | 39.10 | A |
| ATOM | 138 | N | ILE A | 20 | 67.004 | 68.304 | 71.984 | 1.00 | 37.46 | A |
| ATOM | 139 | CA | ILE A | 20 | 65.991 | 67.725 | 71.112 | 1.00 | 37.39 | A |
| ATOM | 140 | CB | ILE A | 20 | 64.685 | 68.503 | 71.168 | 1.00 | 36.72 | A |
| ATOM | 141 | CG2 | ILE A | 20 | 63.687 | 67.918 | 70.167 | 1.00 | 36.45 | A |
| ATOM | 142 | CG1 | ILE A | 20 | 64.118 | 68.440 | 72.572 | 1.00 | 35.02 | A |
| ATOM | 143 | CD1 | ILE A | 20 | 62.910 | 69.280 | 72.751 | 1.00 | 36.54 | A |
| ATOM | 144 | C | ILE A | 20 | 66.432 | 67.678 | 69.660 | 1.00 | 37.89 | A |
| ATOM | 145 | O | ILE A | 20 | 66.157 | 66.718 | 68.954 | 1.00 | 38.16 | A |
| ATOM | 146 | N | ALA A | 21 | 67.125 | 68.717 | 69.218 | 1.00 | 39.24 | A |
| ATOM | 147 | CA | ALA A | 21 | 67.591 | 68.793 | 67.843 | 1.00 | 38.28 | A |
| ATOM | 148 | CB | ALA A | 21 | 68.197 | 70.154 | 67.570 | 1.00 | 38.24 | A |
| ATOM | 149 | C | ALA A | 21 | 68.609 | 67.719 | 67.589 | 1.00 | 38.38 | A |
| ATOM | 150 | O | ALA A | 21 | 68.798 | 67.294 | 66.455 | 1.00 | 38.91 | A |
| ATOM | 151 | N | GLU A | 22 | 69.281 | 67.276 | 68.640 | 1.00 | 38.98 | A |
| ATOM | 152 | CA | GLU A | 22 | 70.280 | 66.242 | 68.449 | 1.00 | 39.28 | A |
| ATOM | 153 | CB | GLU A | 22 | 71.102 | 66.063 | 69.717 | 1.00 | 42.76 | A |
| ATOM | 154 | CG | GLU A | 22 | 72.116 | 67.162 | 69.962 | 1.00 | 47.53 | A |
| ATOM | 155 | CD | GLU A | 22 | 72.856 | 66.948 | 71.259 | 1.00 | 50.36 | A |
| ATOM | 156 | OE1 | GLU A | 22 | 73.381 | 65.827 | 71.444 | 1.00 | 50.70 | A |
| ATOM | 157 | OE2 | GLU A | 22 | 72.907 | 67.891 | 72.089 | 1.00 | 52.73 | A |
| ATOM | 158 | C | GLU A | 22 | 69.666 | 64.913 | 68.021 | 1.00 | 37.58 | A |
| ATOM | 159 | O | GLU A | 22 | 70.391 | 63.955 | 67.805 | 1.00 | 37.40 | A |
| ATOM | 160 | N | ARG A | 23 | 68.342 | 64.850 | 67.890 | 1.00 | 36.03 | A |
| ATOM | 161 | CA | ARG A | 23 | 67.706 | 63.615 | 67.459 | 1.00 | 34.62 | A |
| ATOM | 162 | CB | ARG A | 23 | 66.200 | 63.765 | 67.306 | 1.00 | 33.59 | A |
| ATOM | 163 | CG | ARG A | 23 | 65.509 | 62.431 | 67.022 | 1.00 | 33.69 | A |
| ATOM | 164 | CD | ARG A | 23 | 65.708 | 61.463 | 68.191 | 1.00 | 33.70 | A |
| ATOM | 165 | NE | ARG A | 23 | 64.457 | 61.196 | 68.901 | 1.00 | 34.45 | A |
| ATOM | 166 | CZ | ARG A | 23 | 64.360 | 60.622 | 70.100 | 1.00 | 33.77 | A |
| ATOM | 167 | NH1 | ARG A | 23 | 65.447 | 60.243 | 70.764 | 1.00 | 34.73 | A |
| ATOM | 168 | NH2 | ARG A | 23 | 63.164 | 60.414 | 70.633 | 1.00 | 29.73 | A |
| ATOM | 169 | C | ARG A | 23 | 68.270 | 63.248 | 66.116 | 1.00 | 35.80 | A |
| ATOM | 170 | O | ARG A | 23 | 68.362 | 62.077 | 65.769 | 1.00 | 37.71 | A |
| ATOM | 171 | N | SER A | 24 | 68.629 | 64.266 | 65.345 | 1.00 | 37.45 | A |
| ATOM | 172 | CA | SER A | 24 | 69.208 | 64.072 | 64.018 | 1.00 | 37.69 | A |
| ATOM | 173 | CB | SER A | 24 | 69.047 | 65.344 | 63.187 | 1.00 | 36.12 | A |
| ATOM | 174 | OG | SER A | 24 | 69.929 | 65.337 | 62.084 | 1.00 | 32.96 | A |
| ATOM | 175 | C | SER A | 24 | 70.686 | 63.729 | 64.127 | 1.00 | 38.12 | A |
| ATOM | 176 | O | SER A | 24 | 71.506 | 64.581 | 64.466 | 1.00 | 36.69 | A |
| ATOM | 177 | N | LEU A | 25 | 71.022 | 62.478 | 63.837 | 1.00 | 40.01 | A |
| ATOM | 178 | CA | LEU A | 25 | 72.409 | 62.036 | 63.901 | 1.00 | 42.65 | A |
| ATOM | 179 | CB | LEU A | 25 | 72.534 | 60.580 | 63.436 | 1.00 | 43.67 | A |
| ATOM | 180 | CG | LEU A | 25 | 71.996 | 59.493 | 64.383 | 1.00 | 43.11 | A |
| ATOM | 181 | CD1 | LEU A | 25 | 72.208 | 58.104 | 63.794 | 1.00 | 41.75 | A |
| ATOM | 182 | CD2 | LEU A | 25 | 72.703 | 59.615 | 65.713 | 1.00 | 41.72 | A |
| ATOM | 183 | C | LEU A | 25 | 73.313 | 62.916 | 63.054 | 1.00 | 42.76 | A |
| ATOM | 184 | O | LEU A | 25 | 74.475 | 63.112 | 63.394 | 1.00 | 42.58 | A |
| ATOM | 185 | N | LEU A | 26 | 72.779 | 63.442 | 61.954 | 1.00 | 44.29 | A |
| ATOM | 186 | CA | LEU A | 26 | 73.554 | 64.309 | 61.070 | 1.00 | 46.11 | A |
| ATOM | 187 | CB | LEU A | 26 | 72.701 | 64.742 | 59.875 | 1.00 | 46.16 | A |
| ATOM | 188 | CG | LEU A | 26 | 73.318 | 65.721 | 58.868 | 1.00 | 45.56 | A |
| ATOM | 189 | CD1 | LED A | 26 | 74.571 | 65.123 | 58.268 | 1.00 | 44.90 | A |
| ATOM | 190 | CD2 | LED A | 26 | 72.303 | 66.050 | 57.774 | 1.00 | 45.90 | A |
| ATOM | 191 | C | LED A | 26 | 73.992 | 65.524 | 61.866 | 1.00 | 47.66 | A |
| ATOM | 192 | O | LED A | 26 | 75.140 | 65.962 | 61.770 | 1.00 | 47.34 | A |
| ATOM | 193 | N | LEU A | 27 | 73.060 | 66.055 | 62.655 | 1.00 | 50.27 | A |
| ATOM | 194 | CA | LED A | 27 | 73.312 | 67.212 | 63.508 | 1.00 | 52.62 | A |
| ATOM | 195 | CB | LEU A | 27 | 71.985 | 67.831 | 63.963 | 1.00 | 52.60 | A |
| ATOM | 196 | CG | LED A | 27 | 72.112 | 68.911 | 65.046 | 1.00 | 52.80 | A |
| ATOM | 197 | CD1 | LEU A | 27 | 72.872 | 70.114 | 64.501 | 1.00 | 52.04 | A |
| ATOM | 198 | CD2 | LED A | 27 | 70.729 | 69.314 | 65.513 | 1.00 | 52.96 | A |
| ATOM | 199 | C | LED A | 27 | 74.145 | 66.835 | 64.742 | 1.00 | 54.44 | A |
| ATOM | 200 | O | LED A | 27 | 75.085 | 67.540 | 65.100 | 1.00 | 54.34 | A |
| ATOM | 201 | N | LYS A | 28 | 73.786 | 65.726 | 65.387 | 1.00 | 56.05 | A |
| ATOM | 202 | CA | LYS A | 28 | 74.491 | 65.255 | 66.574 | 1.00 | 57.53 | A |
| ATOM | 203 | CB | LYS A | 28 | 73.880 | 63.942 | 67.059 | 1.00 | 58.27 | A |
| ATOM | 204 | CG | LYS A | 28 | 74.486 | 63.418 | 68.347 | 0.00 | 58.77 | A |
| ATOM | 205 | CD | LYS A | 28 | 73.864 | 62.093 | 68.750 | 0.00 | 59.35 | A |
| ATOM | 206 | CE | LYS A | 28 | 74.520 | 61.545 | 70.004 | 0.00 | 59.70 | A |
| ATOM | 207 | NZ | LYS A | 28 | 73.986 | 60.205 | 70.367 | 0.00 | 59.95 | A |
| ATOM | 208 | C | LYS A | 28 | 75.974 | 65.049 | 66.297 | 0.00 | 58.99 | A |
| ATOM | 209 | O | LYS A | 28 | 76.811 | 65.183 | 67.191 | 0.00 | 58.95 | A |
| ATOM | 210 | N | ASN A | 29 | 76.290 | 64.716 | 65.052 | 1.00 | 60.36 | A |
| ATOM | 211 | CA | ASN A | 29 | 77.667 | 64.489 | 64.647 | 1.00 | 62.38 | A |
| ATOM | 212 | CB | ASN A | 29 | 77.729 | 63.509 | 63.484 | 1.00 | 63.15 | A |
| ATOM | 213 | CG | ASN A | 29 | 77.458 | 62.085 | 63.908 | 1.00 | 64.88 | A |
| ATOM | 214 | OD1 | ASN A | 29 | 77.410 | 61.185 | 63.068 | 1.00 | 67.16 | A |
| ATOM | 215 | ND2 | ASN A | 29 | 77.285 | 61.865 | 65.215 | 1.00 | 64.20 | A |
| ATOM | 216 | C | ASN A | 29 | 78.326 | 65.778 | 64.227 | 1.00 | 63.19 | A |
| ATOM | 217 | O | ASN A | 29 | 79.547 | 65.852 | 64.106 | 1.00 | 63.26 | A |
| ATOM | 218 | N | TYR A | 30 | 77.514 | 66.797 | 63.993 | 1.00 | 64.46 | A |
| ATOM | 219 | CA | TYR A | 30 | 78.049 | 68.083 | 63.583 | 1.00 | 65.53 | A |
| ATOM | 220 | CB | TYR A | 30 | 76.898 | 69.085 | 63.417 | 1.00 | 67.82 | A |
| ATOM | 221 | CG | TYR A | 30 | 77.127 | 70.172 | 62.379 | 1.00 | 68.70 | A |
| ATOM | 222 | CD1 | TYR A | 30 | 77.416 | 69.853 | 61.050 | 1.00 | 68.47 | A |
| ATOM | 223 | CE1 | TYR A | 30 | 77.595 | 70.854 | 60.092 | 1.00 | 69.31 | A |
| ATOM | 224 | CD2 | TYR A | 30 | 77.024 | 71.522 | 62.722 | 1.00 | 68.53 | A |
| ATOM | 225 | CE2 | TYR A | 30 | 77.201 | 72.528 | 61.774 | 1.00 | 69.17 | A |
| ATOM | 226 | CZ | TYR A | 30 | 77.485 | 72.190 | 60.464 | 1.00 | 69.54 | A |
| ATOM | 227 | OH | TYR A | 30 | 77.651 | 73.191 | 59.534 | 1.00 | 69.67 | A |
| ATOM | 228 | C | TYR A | 30 | 79.043 | 68.550 | 64.658 | 1.00 | 65.21 | A |
| ATOM | 229 | O | TYR A | 30 | 79.894 | 69.395 | 64.391 | 1.00 | 64.91 | A |
| ATOM | 230 | N | VAL A | 31 | 78.935 | 67.964 | 65.856 | 1.00 | 64.85 | A |
| ATOM | 231 | CA | VAL A | 31 | 79.786 | 68.271 | 67.019 | 1.00 | 62.98 | A |
| ATOM | 232 | CB | VAL A | 31 | 81.049 | 67.351 | 67.096 | 1.00 | 62.18 | A |
| ATOM | 233 | CG1 | VAL A | 31 | 80.624 | 65.903 | 67.216 | 1.00 | 61.36 | A |
| ATOM | 234 | CG2 | VAL A | 31 | 81.961 | 67.562 | 65.882 | 1.00 | 60.58 | A |
| ATOM | 235 | C | VAL A | 31 | 80.256 | 69.712 | 67.123 | 1.00 | 62.08 | A |
| ATOM | 236 | O | VAL A | 31 | 80.624 | 70.168 | 68.203 | 1.00 | 60.74 | A |
| ATOM | 237 | N | ILE A | 45 | 76.524 | 76.394 | 65.711 | 1.00 | 42.05 | A |
| ATOM | 238 | CA | ILE A | 45 | 75.216 | 76.668 | 66.290 | 1.00 | 41.90 | A |
| ATOM | 239 | CB | ILE A | 45 | 75.276 | 77.864 | 67.310 | 1.00 | 42.05 | A |
| ATOM | 240 | CG2 | ILE A | 45 | 75.089 | 79.191 | 66.601 | 1.00 | 40.06 | A |
| ATOM | 241 | CG1 | ILE A | 45 | 74.171 | 77.726 | 68.363 | 1.00 | 43.22 | A |
| ATOM | 242 | CD1 | ILE A | 45 | 74.407 | 76.620 | 69.388 | 1.00 | 43.84 | A |
| ATOM | 243 | C | ILE A | 45 | 74.180 | 76.963 | 65.195 | 1.00 | 41.36 | A |
| ATOM | 244 | O | ILE A | 45 | 73.021 | 77.252 | 65.488 | 1.00 | 41.48 | A |
| ATOM | 245 | N | VAL A | 46 | 74.592 | 76.918 | 63.933 | 1.00 | 39.64 | A |
| ATOM | 246 | CA | VAL A | 46 | 73.635 | 77.132 | 62.849 | 1.00 | 39.19 | A |
| ATOM | 247 | CB | VAL A | 46 | 73.675 | 78.579 | 62.293 | 1.00 | 37.60 | A |
| ATOM | 248 | CG1 | VAL A | 46 | 72.665 | 78.729 | 61.166 | 1.00 | 35.40 | A |
| ATOM | 249 | CG2 | VAL A | 46 | 73.346 | 79.567 | 63.390 | 1.00 | 38.18 | A |
| ATOM | 250 | C | VAL A | 46 | 73.917 | 76.159 | 61.715 | 1.00 | 39.41 | A |
| ATOM | 251 | O | VAL A | 46 | 74.815 | 76.385 | 60.905 | 1.00 | 41.28 | A |
| ATOM | 252 | N | MSE A | 47 | 73.160 | 75.069 | 61.655 | 1.00 | 38.13 | A |
| ATOM | 253 | CA | MSE A | 47 | 73.390 | 74.093 | 60.606 | 1.00 | 37.60 | A |
| ATOM | 254 | CB | MSE A | 47 | 73.419 | 72.680 | 61.169 | 1.00 | 40.79 | A |
| ATOM | 255 | CG | MSE A | 47 | 73.777 | 71.642 | 60.118 | 1.00 | 44.02 | A |
| ATOM | 256 | SE | MSE A | 47 | 73.314 | 69.875 | 60.679 | 1.00 | 50.37 | A |
| ATOM | 257 | CE | MSE A | 47 | 71.388 | 70.111 | 60.653 | 1.00 | 48.04 | A |
| ATOM | 258 | C | MSE A | 47 | 72.402 | 74.138 | 59.465 | 1.00 | 36.07 | A |
| ATOM | 259 | O | MSE A | 47 | 71.195 | 74.213 | 59.670 | 1.00 | 36.45 | A |
| ATOM | 260 | N | PRO A | 48 | 72.917 | 74.093 | 58.229 | 1.00 | 34.97 | A |
| ATOM | 261 | CD | PRO A | 48 | 74.305 | 74.434 | 57.870 | 1.00 | 34.19 | A |
| ATOM | 262 | CA | PRO A | 48 | 72.066 | 74.128 | 57.040 | 1.00 | 34.14 | A |
| ATOM | 263 | CB | PRO A | 48 | 73.041 | 74.516 | 55.929 | 1.00 | 33.07 | A |
| ATOM | 264 | CG | PRO A | 48 | 74.103 | 75.290 | 56.654 | 1.00 | 33.93 | A |
| ATOM | 265 | C | PRO A | 48 | 71.369 | 72.806 | 56.729 | 1.00 | 33.52 | A |
| ATOM | 266 | O | PRO A | 48 | 71.922 | 71.727 | 56.946 | 1.00 | 32.95 | A |
| ATOM | 267 | N | VAL A | 49 | 70.140 | 72.906 | 56.231 | 1.00 | 32.78 | A |
| ATOM | 268 | CA | VAL A | 49 | 69.383 | 71.731 | 55.828 | 1.00 | 32.43 | A |
| ATOM | 269 | CB | VAL A | 49 | 68.073 | 71.538 | 56.671 | 1.00 | 31.33 | A |
| ATOM | 270 | CG1 | VAL A | 49 | 67.575 | 70.120 | 56.539 | 1.00 | 30.50 | A |
| ATOM | 271 | CG2 | VAL A | 49 | 68.327 | 71.845 | 58.141 | 1.00 | 34.13 | A |
| ATOM | 272 | C | VAL A | 49 | 69.062 | 71.991 | 54.340 | 1.00 | 32.95 | A |
| ATOM | 273 | O | VAL A | 49 | 67.958 | 72.412 | 53.977 | 1.00 | 32.24 | A |
| ATOM | 274 | N | PRO A | 50 | 70.060 | 71.757 | 53.460 | 1.00 | 33.40 | A |
| ATOM | 275 | CD | PRO A | 50 | 71.393 | 71.237 | 53.811 | 1.00 | 33.34 | A |
| ATOM | 276 | CA | PRO A | 50 | 69.971 | 71.942 | 52.010 | 1.00 | 32.39 | A |
| ATOM | 277 | CB | PRO A | 50 | 71.310 | 71.409 | 51.512 | 1.00 | 32.52 | A |
| ATOM | 278 | CG | PRO A | 50 | 72.217 | 71.682 | 52.627 | 1.00 | 33.15 | A |
| ATOM | 279 | C | PRO A | 50 | 68.813 | 71.229 | 51.343 | 1.00 | 31.05 | A |
| ATOM | 280 | O | PRO A | 50 | 68.509 | 70.079 | 51.648 | 1.00 | 31.50 | A |
| ATOM | 281 | N | ASN A | 51 | 68.176 | 71.936 | 50.420 | 1.00 | 30.37 | A |
| ATOM | 282 | CA | ASN A | 51 | 67.071 | 71.387 | 49.645 | 1.00 | 30.66 | A |
| ATOM | 283 | CB | ASN A | 51 | 67.648 | 70.432 | 48.595 | 1.00 | 31.76 | A |
| ATOM | 284 | CG | ASN A | 51 | 69.069 | 70.813 | 48.175 | 1.00 | 30.85 | A |
| ATOM | 285 | OD1 | ASN A | 51 | 69.262 | 71.794 | 47.478 | 1.00 | 27.98 | A |
| ATOM | 286 | ND2 | ASN A | 51 | 70.072 | 70.031 | 48.620 | 1.00 | 33.14 | A |
| ATOM | 287 | C | ASN A | 51 | 65.978 | 70.665 | 50.450 | 1.00 | 29.20 | A |
| ATOM | 288 | O | ASN A | 51 | 65.588 | 69.551 | 50.120 | 1.00 | 26.62 | A |
| ATOM | 289 | N | VAL A | 52 | 65.500 | 71.292 | 51.512 | 1.00 | 29.33 | A |
| ATOM | 290 | CA | VAL A | 52 | 64.435 | 70.692 | 52.283 | 1.00 | 29.38 | A |
| ATOM | 291 | CB | VAL A | 52 | 64.913 | 70.157 | 53.640 | 1.00 | 30.20 | A |
| ATOM | 292 | CG1 | VAL A | 52 | 63.747 | 69.506 | 54.390 | 1.00 | 26.29 | A |
| ATOM | 293 | CG2 | VAL A | 52 | 66.022 | 69.129 | 53.420 | 1.00 | 29.10 | A |
| ATOM | 294 | C | VAL A | 52 | 63.401 | 71.774 | 52.492 | 1.00 | 29.83 | A |
| ATOM | 295 | O | VAL A | 52 | 63.693 | 72.821 | 53.058 | 1.00 | 29.62 | A |
| ATOM | 296 | N | ARG A | 53 | 62.199 | 71.504 | 51.992 | 1.00 | 30.70 | A |
| ATOM | 297 | CA | ARG A | 53 | 61.064 | 72.411 | 52.063 | 1.00 | 31.16 | A |
| ATOM | 298 | CB | ARG A | 53 | 59.814 | 71.667 | 51.580 | 1.00 | 35.30 | A |
| ATOM | 299 | CG | ARG A | 53 | 58.775 | 72.499 | 50.846 | 1.00 | 39.79 | A |
| ATOM | 300 | CD | ARG A | 53 | 57.564 | 71.636 | 50.474 | 1.00 | 42.92 | A |
| ATOM | 301 | NE | ARG A | 53 | 56.700 | 72.283 | 49.491 | 1.00 | 47.91 | A |
| ATOM | 302 | CZ | ARG A | 53 | 55.889 | 73.308 | 49.749 | 1.00 | 50.95 | A |
| ATOM | 303 | NH1 | ARG A | 53 | 55.815 | 73.814 | 50.972 | 1.00 | 53.87 | A |
| ATOM | 304 | NH2 | ARC A | 53 | 55.158 | 73.844 | 48.776 | 1.00 | 52.85 | A |
| ATOM | 305 | C | ARC A | 53 | 60.864 | 72.876 | 53.505 | 1.00 | 30.38 | A |
| ATOM | 306 | O | ARC A | 53 | 60.860 | 72.068 | 54.425 | 1.00 | 31.84 | A |
| ATOM | 307 | N | SER A | 54 | 60.694 | 74.173 | 53.708 | 1.00 | 27.65 | A |
| ATOM | 308 | CA | SER A | 54 | 60.481 | 74.670 | 55.050 | 1.00 | 24.25 | A |
| ATOM | 309 | CB | SER A | 54 | 60.094 | 76.142 | 55.013 | 1.00 | 23.48 | A |
| ATOM | 310 | OC | SER A | 54 | 61.197 | 76.935 | 54.642 | 1.00 | 22.07 | A |
| ATOM | 311 | C | SER A | 54 | 59.386 | 73.866 | 55.742 | 1.00 | 22.74 | A |
| ATOM | 312 | O | SER A | 54 | 59.611 | 73.280 | 56.792 | 1.00 | 21.74 | A |
| ATOM | 313 | N | SER A | 55 | 58.204 | 73.835 | 55.143 | 1.00 | 22.72 | A |
| ATOM | 314 | CA | SER A | 55 | 57.078 | 73.114 | 55.707 | 1.00 | 22.82 | A |
| ATOM | 315 | CB | SER A | 55 | 55.913 | 73.117 | 54.736 | 1.00 | 20.88 | A |
| ATOM | 316 | OC | SER A | 55 | 56.159 | 72.211 | 53.683 | 1.00 | 23.15 | A |
| ATOM | 317 | C | SER A | 55 | 57.432 | 71.670 | 56.042 | 1.00 | 24.40 | A |
| ATOM | 318 | O | SER A | 55 | 56.780 | 71.034 | 56.877 | 1.00 | 27.21 | A |
| ATOM | 319 | N | VAL A | 56 | 58.457 | 71.144 | 55.387 | 1.00 | 21.45 | A |
| ATOM | 320 | CA | VAL A | 56 | 58.862 | 69.782 | 55.662 | 1.00 | 20.79 | A |
| ATOM | 321 | CB | VAL A | 56 | 59.648 | 69.194 | 54.488 | 1.00 | 19.14 | A |
| ATOM | 322 | CG1 | VAL A | 56 | 60.254 | 67.875 | 54.889 | 1.00 | 16.99 | A |
| ATOM | 323 | CG2 | VAL A | 56 | 58.738 | 69.029 | 53.301 | 1.00 | 18.04 | A |
| ATOM | 324 | C | VAL A | 56 | 59.719 | 69.727 | 56.924 | 1.00 | 21.47 | A |
| ATOM | 325 | O | VAL A | 56 | 59.498 | 68.903 | 57.797 | 1.00 | 21.73 | A |
| ATOM | 326 | N | LEU A | 57 | 60.703 | 70.606 | 57.010 | 1.00 | 22.90 | A |
| ATOM | 327 | CA | LEU A | 57 | 61.573 | 70.645 | 58.159 | 1.00 | 24.53 | A |
| ATOM | 328 | CB | LEU A | 57 | 62.647 | 71.696 | 57.937 | 1.00 | 24.08 | A |
| ATOM | 329 | CG | LEU A | 57 | 63.681 | 71.859 | 59.042 | 1.00 | 24.59 | A |
| ATOM | 330 | CD1 | LEU A | 57 | 64.030 | 70.523 | 59.664 | 1.00 | 23.09 | A |
| ATOM | 331 | CD2 | LEU A | 57 | 64.924 | 72.530 | 58.438 | 1.00 | 23.83 | A |
| ATOM | 332 | C | LEU A | 57 | 60.760 | 70.949 | 59.411 | 1.00 | 27.00 | A |
| ATOM | 333 | O | LEU A | 57 | 61.044 | 70.424 | 60.486 | 1.00 | 27.31 | A |
| ATOM | 334 | N | GLN A | 58 | 59.740 | 71.790 | 59.270 | 1.00 | 28.60 | A |
| ATOM | 335 | CA | GLN A | 58 | 58.885 | 72.133 | 60.402 | 1.00 | 29.92 | A |
| ATOM | 336 | CB | GLN A | 58 | 57.789 | 73.112 | 59.979 | 1.00 | 29.50 | A |
| ATOM | 337 | CG | GLN A | 58 | 56.729 | 73.358 | 61.044 | 1.00 | 29.08 | A |
| ATOM | 338 | CD | GLN A | 58 | 55.975 | 74.654 | 60.815 | 1.00 | 27.90 | A |
| ATOM | 339 | OE1 | GLN A | 58 | 56.591 | 75.700 | 60.657 | 1.00 | 30.17 | A |
| ATOM | 340 | NE2 | GLN A | 58 | 54.648 | 74.594 | 60.802 | 1.00 | 25.55 | A |
| ATOM | 341 | C | GLN A | 58 | 58.257 | 70.845 | 60.886 | 1.00 | 30.93 | A |
| ATOM | 342 | O | GLN A | 58 | 58.354 | 70.483 | 62.065 | 1.00 | 33.35 | A |
| ATOM | 343 | N | LYS A | 59 | 57.609 | 70.156 | 59.955 | 1.00 | 30.29 | A |
| ATOM | 344 | CA | LYS A | 59 | 56.976 | 68.880 | 60.248 | 1.00 | 29.48 | A |
| ATOM | 345 | CB | LYS A | 59 | 56.581 | 68.209 | 58.937 | 1.00 | 28.21 | A |
| ATOM | 346 | CG | LYS A | 59 | 55.283 | 67.470 | 58.978 | 1.00 | 29.03 | A |
| ATOM | 347 | CD | LYS A | 59 | 54.108 | 68.397 | 59.081 | 1.00 | 27.32 | A |
| ATOM | 348 | CE | LYS A | 59 | 52.834 | 67.608 | 58.857 | 1.00 | 26.80 | A |
| ATOM | 349 | NZ | LYS A | 59 | 52.804 | 66.508 | 59.824 | 1.00 | 25.00 | A |
| ATOM | 350 | C | LYS A | 59 | 58.004 | 68.017 | 61.006 | 1.00 | 29.46 | A |
| ATOM | 351 | O | LYS A | 59 | 57.709 | 67.465 | 62.070 | 1.00 | 29.02 | A |
| ATOM | 352 | N | VAL A | 60 | 59.217 | 67.929 | 60.467 | 1.00 | 28.15 | A |
| ATOM | 353 | CA | VAL A | 60 | 60.259 | 67.142 | 61.106 | 1.00 | 28.14 | A |
| ATOM | 354 | CB | VAL A | 60 | 61.573 | 67.226 | 60.319 | 1.00 | 28.15 | A |
| ATOM | 355 | CG1 | VAL A | 60 | 62.731 | 66.715 | 61.152 | 1.00 | 27.46 | A |
| ATOM | 356 | CG2 | VAL A | 60 | 61.455 | 66.386 | 59.060 | 1.00 | 29.05 | A |
| ATOM | 357 | C | VAL A | 60 | 60.495 | 67.574 | 62.543 | 1.00 | 28.57 | A |
| ATOM | 358 | O | VAL A | 60 | 60.434 | 66.762 | 63.465 | 1.00 | 29.28 | A |
| ATOM | 359 | N | ILE A | 61 | 60.768 | 68.853 | 62.734 | 1.00 | 28.79 | A |
| ATOM | 360 | CA | ILE A | 61 | 61.007 | 69.375 | 64.068 | 1.00 | 28.54 | A |
| ATOM | 361 | CB | ILE A | 61 | 61.195 | 70.903 | 64.001 | 1.00 | 27.31 | A |
| ATOM | 362 | CG2 | ILE A | 61 | 61.238 | 71.501 | 65.391 | 1.00 | 26.36 | A |
| ATOM | 363 | CG1 | ILE A | 61 | 62.482 | 71.202 | 63.223 | 1.00 | 27.68 | A |
| ATOM | 364 | CD1 | ILE A | 61 | 62.737 | 72.660 | 62.979 | 1.00 | 27.54 | A |
| ATOM | 365 | C | ILE A | 61 | 59.860 | 69.020 | 65.016 | 1.00 | 29.35 | A |
| ATOM | 366 | O | ILE A | 61 | 60.086 | 68.538 | 66.131 | 1.00 | 27.58 | A |
| ATOM | 367 | N | GLU A | 62 | 58.627 | 69.247 | 64.572 | 1.00 | 30.00 | A |
| ATOM | 368 | CA | GLU A | 62 | 57.473 | 68.949 | 65.415 | 1.00 | 31.73 | A |
| ATOM | 369 | CB | GLU A | 62 | 56.169 | 69.203 | 64.667 | 1.00 | 29.92 | A |
| ATOM | 370 | CG | GLU A | 62 | 55.043 | 68.328 | 65.181 | 1.00 | 30.57 | A |
| ATOM | 371 | CD | GLU A | 62 | 53.688 | 68.775 | 64.703 | 1.00 | 31.86 | A |
| ATOM | 372 | OE1 | GLU A | 62 | 53.530 | 68.966 | 63.470 | 1.00 | 33.09 | A |
| ATOM | 373 | OE2 | GLU A | 62 | 52.781 | 68.934 | 65.562 | 1.00 | 31.63 | A |
| ATOM | 374 | C | GLU A | 62 | 57.480 | 67.505 | 65.907 | 1.00 | 32.78 | A |
| ATOM | 375 | O | GLU A | 62 | 57.035 | 67.207 | 67.017 | 1.00 | 32.73 | A |
| ATOM | 376 | N | TRP A | 63 | 57.965 | 66.610 | 65.057 | 1.00 | 34.15 | A |
| ATOM | 377 | CA | TRP A | 63 | 58.029 | 65.204 | 65.394 | 1.00 | 34.37 | A |
| ATOM | 378 | CB | TRP A | 63 | 58.259 | 64.380 | 64.127 | 1.00 | 33.74 | A |
| ATOM | 379 | CG | TRP A | 63 | 58.189 | 62.915 | 64.354 | 1.00 | 31.94 | A |
| ATOM | 380 | CD2 | TRP A | 63 | 59.254 | 62.071 | 64.798 | 1.00 | 30.34 | A |
| ATOM | 381 | CE2 | TRP A | 63 | 58.729 | 60.775 | 64.937 | 1.00 | 29.79 | A |
| ATOM | 382 | CE3 | TRP A | 63 | 60.601 | 62.287 | 65.104 | 1.00 | 29.39 | A |
| ATOM | 383 | CD1 | TRP A | 63 | 57.092 | 62.120 | 64.235 | 1.00 | 31.15 | A |
| ATOM | 384 | NE1 | TRP A | 63 | 57.406 | 60.829 | 64.584 | 1.00 | 30.29 | A |
| ATOM | 385 | CZ2 | TRP A | 63 | 59.506 | 59.698 | 65.361 | 1.00 | 29.60 | A |
| ATOM | 386 | CZ3 | TRP A | 63 | 61.372 | 61.211 | 65.528 | 1.00 | 28.85 | A |
| ATOM | 387 | CH2 | TRP A | 63 | 60.821 | 59.937 | 65.654 | 1.00 | 27.95 | A |
| ATOM | 388 | C | TRP A | 63 | 59.181 | 64.989 | 66.372 | 1.00 | 34.92 | A |
| ATOM | 389 | O | TRP A | 63 | 59.146 | 64.078 | 67.182 | 1.00 | 36.00 | A |
| ATOM | 390 | N | ALA A | 64 | 60.195 | 65.840 | 66.316 | 1.00 | 35.06 | A |
| ATOM | 391 | CA | ALA A | 64 | 61.332 | 65.673 | 67.207 | 1.00 | 36.52 | A |
| ATOM | 392 | CB | ALA A | 64 | 62.567 | 66.371 | 66.621 | 1.00 | 35.61 | A |
| ATOM | 393 | C | ALA A | 64 | 61.071 | 66.166 | 68.636 | 1.00 | 38.06 | A |
| ATOM | 394 | O | ALA A | 64 | 61.606 | 65.607 | 69.602 | 1.00 | 38.15 | A |
| ATOM | 395 | N | GLU A | 65 | 60.266 | 67.218 | 68.764 | 1.00 | 38.68 | A |
| ATOM | 396 | CA | GLU A | 65 | 59.934 | 67.776 | 70.063 | 1.00 | 38.24 | A |
| ATOM | 397 | CB | GLU A | 65 | 59.405 | 69.201 | 69.912 | 1.00 | 38.18 | A |
| ATOM | 398 | CG | GLU A | 65 | 60.459 | 70.151 | 69.397 | 1.00 | 38.80 | A |
| ATOM | 399 | CD | GLU A | 65 | 59.895 | 71.454 | 68.840 | 1.00 | 39.78 | A |
| ATOM | 400 | OE1 | GLU A | 65 | 58.673 | 71.527 | 68.547 | 1.00 | 38.29 | A |
| ATOM | 401 | OE2 | GLU A | 65 | 60.695 | 72.408 | 68.683 | 1.00 | 39.80 | A |
| ATOM | 402 | C | GLU A | 65 | 58.879 | 66.902 | 70.708 | 1.00 | 38.37 | A |
| ATOM | 403 | O | GLU A | 65 | 58.835 | 66.742 | 71.925 | 1.00 | 39.26 | A |
| ATOM | 404 | N | HIS A | 66 | 58.024 | 66.312 | 69.896 | 1.00 | 38.38 | A |
| ATOM | 405 | CA | HIS A | 66 | 57.006 | 65.482 | 70.479 | 1.00 | 40.16 | A |
| ATOM | 406 | CB | HIS A | 66 | 55.929 | 65.179 | 69.465 | 1.00 | 39.56 | A |
| ATOM | 407 | CG | HIS A | 66 | 54.902 | 64.215 | 69.955 | 1.00 | 39.86 | A |
| ATOM | 408 | CD2 | HIS A | 66 | 53.613 | 64.399 | 70.320 | 1.00 | 39.39 | A |
| ATOM | 409 | ND1 | HIS A | 66 | 55.137 | 62.859 | 70.036 | 1.00 | 38.45 | A |
| ATOM | 410 | CE1 | HIS A | 66 | 54.031 | 62.248 | 70.418 | 1.00 | 38.21 | A |
| ATOM | 411 | NE2 | HIS A | 66 | 53.091 | 63.159 | 70.595 | 1.00 | 39.69 | A |
| ATOM | 412 | C | HIS A | 66 | 57.616 | 64.205 | 71.002 | 1.00 | 42.15 | A |
| ATOM | 413 | O | HIS A | 66 | 57.115 | 63.619 | 71.959 | 1.00 | 44.74 | A |
| ATOM | 414 | N | HIS A | 67 | 58.701 | 63.775 | 70.377 | 1.00 | 43.11 | A |
| ATOM | 415 | CA | HIS A | 67 | 59.388 | 62.573 | 70.807 | 1.00 | 44.64 | A |
| ATOM | 416 | CB | HIS A | 67 | 59.711 | 61.681 | 69.598 | 1.00 | 43.57 | A |
| ATOM | 417 | CG | HIS A | 67 | 58.524 | 60.951 | 69.046 | 1.00 | 42.88 | A |
| ATOM | 418 | CD2 | HIS A | 67 | 58.088 | 59.684 | 69.241 | 1.00 | 42.35 | A |
| ATOM | 419 | ND1 | HIS A | 67 | 57.615 | 61.540 | 68.194 | 1.00 | 43.39 | A |
| ATOM | 420 | CE1 | HIS A | 67 | 56.673 | 60.667 | 67.886 | 1.00 | 41.93 | A |
| ATOM | 421 | NE2 | HIS A | 67 | 56.936 | 59.533 | 68.508 | 1.00 | 40.92 | A |
| ATOM | 422 | C | HIS A | 67 | 60.673 | 62.989 | 71.512 | 1.00 | 46.18 | A |
| ATOM | 423 | O | HIS A | 67 | 61.767 | 62.552 | 71.144 | 1.00 | 47.55 | A |
| ATOM | 424 | N | ARG A | 68 | 60.541 | 63.842 | 72.524 | 1.00 | 47.19 | A |
| ATOM | 425 | CA | ARG A | 68 | 61.704 | 64.322 | 73.270 | 1.00 | 48.05 | A |
| ATOM | 426 | CB | ARG A | 68 | 61.390 | 65.665 | 73.959 | 1.00 | 47.55 | A |
| ATOM | 427 | CG | ARG A | 68 | 60.459 | 65.576 | 75.163 | 1.00 | 47.12 | A |
| ATOM | 428 | CD | ARG A | 68 | 60.219 | 66.943 | 75.813 | 1.00 | 45.40 | A |
| ATOM | 429 | NE | ARG A | 68 | 59.137 | 67.657 | 75.151 | 1.00 | 44.72 | A |
| ATOM | 430 | CZ | ARG A | 68 | 57.849 | 67.387 | 75.342 | 1.00 | 44.29 | A |
| ATOM | 431 | NH1 | ARG A | 68 | 57.485 | 66.434 | 76.179 | 1.00 | 46.93 | A |
| ATOM | 432 | NH2 | ARG A | 68 | 56.915 | 68.043 | 74.678 | 1.00 | 45.61 | A |
| ATOM | 433 | C | ARG A | 68 | 62.187 | 63.308 | 74.303 | 1.00 | 49.19 | A |
| ATOM | 434 | O | ARG A | 68 | 63.383 | 63.228 | 74.593 | 1.00 | 48.48 | A |
| ATOM | 435 | N | ASP A | 69 | 61.261 | 62.525 | 74.848 | 1.00 | 51.12 | A |
| ATOM | 436 | CA | ASP A | 69 | 61.626 | 61.531 | 75.853 | 1.00 | 52.05 | A |
| ATOM | 437 | CB | ASP A | 69 | 60.967 | 61.862 | 77.187 | 1.00 | 50.12 | A |
| ATOM | 438 | CG | ASP A | 69 | 61.351 | 63.221 | 77.684 | 1.00 | 49.36 | A |
| ATOM | 439 | OD1 | ASP A | 69 | 62.572 | 63.496 | 77.736 | 1.00 | 48.50 | A |
| ATOM | 440 | OD2 | ASP A | 69 | 60.440 | 64.010 | 78.015 | 1.00 | 49.39 | A |
| ATOM | 441 | C | ASP A | 69 | 61.245 | 60.126 | 75.442 | 1.00 | 52.86 | A |
| ATOM | 442 | O | ASP A | 69 | 60.787 | 59.335 | 76.261 | 1.00 | 52.94 | A |
| ATOM | 443 | N | SER A | 70 | 61.437 | 59.812 | 74.170 | 1.00 | 54.02 | A |
| ATOM | 444 | CA | SER A | 70 | 61.106 | 58.488 | 73.690 | 1.00 | 55.97 | A |
| ATOM | 445 | CB | SER A | 70 | 60.259 | 58.588 | 72.425 | 1.00 | 54.70 | A |
| ATOM | 446 | OG | SER A | 70 | 59.031 | 59.239 | 72.703 | 1.00 | 54.60 | A |
| ATOM | 447 | C | SER A | 70 | 62.379 | 57.702 | 73.425 | 1.00 | 58.22 | A |
| ATOM | 448 | O | SER A | 70 | 63.463 | 58.273 | 73.306 | 1.00 | 58.71 | A |
| ATOM | 449 | N | ASN A | 71 | 62.252 | 56.385 | 73.362 | 1.00 | 60.80 | A |
| ATOM | 450 | CA | ASN A | 71 | 63.403 | 55.545 | 73.105 | 1.00 | 64.11 | A |
| ATOM | 451 | CB | ASN A | 71 | 63.999 | 55.049 | 74.421 | 1.00 | 62.74 | A |
| ATOM | 452 | CG | ASN A | 71 | 64.012 | 56.121 | 75.495 | 1.00 | 62.42 | A |
| ATOM | 453 | OD1 | ASN A | 71 | 63.016 | 56.332 | 76.183 | 1.00 | 61.70 | A |
| ATOM | 454 | ND2 | ASN A | 71 | 65.139 | 56.811 | 75.636 | 1.00 | 62.41 | A |
| ATOM | 455 | C | ASN A | 71 | 62.957 | 54.377 | 72.247 | 1.00 | 67.50 | A |
| ATOM | 456 | O | ASN A | 71 | 62.140 | 53.558 | 72.669 | 1.00 | 68.91 | A |
| ATOM | 457 | N | PHE A | 72 | 63.486 | 54.313 | 71.030 | 1.00 | 70.54 | A |
| ATOM | 458 | CA | PHE A | 72 | 63.134 | 53.245 | 70.110 | 1.00 | 72.15 | A |
| ATOM | 459 | CB | PHE A | 72 | 62.792 | 53.827 | 68.721 | 1.00 | 72.15 | A |
| ATOM | 460 | CG | PHE A | 72 | 61.940 | 55.087 | 68.759 | 1.00 | 71.12 | A |
| ATOM | 461 | CD1 | PHE A | 72 | 62.539 | 56.349 | 68.784 | 1.00 | 70.74 | A |
| ATOM | 462 | CD2 | PHE A | 72 | 60.546 | 55.012 | 68.765 | 1.00 | 70.32 | A |
| ATOM | 463 | CE1 | PHE A | 72 | 61.767 | 57.514 | 68.814 | 1.00 | 70.31 | A |
| ATOM | 464 | CE2 | PHE A | 72 | 59.768 | 56.173 | 68.796 | 1.00 | 70.64 | A |
| ATOM | 465 | CZ | PHE A | 72 | 60.383 | 57.425 | 68.821 | 1.00 | 70.21 | A |
| ATOM | 466 | C | PHE A | 72 | 64.307 | 52.261 | 70.001 | 1.00 | 73.60 | A |
| ATOM | 467 | O | PHE A | 72 | 65.466 | 52.623 | 70.226 | 1.00 | 73.93 | A |
| ATOM | 468 | N | PRO A | 73 | 64.012 | 50.992 | 69.685 | 1.00 | 75.25 | A |
| ATOM | 469 | CD | PRO A | 73 | 62.656 | 50.410 | 69.635 | 1.00 | 75.90 | A |
| ATOM | 470 | CA | PRO A | 73 | 65.039 | 49.952 | 69.545 | 1.00 | 75.83 | A |
| ATOM | 471 | CB | PRO A | 73 | 64.230 | 48.732 | 69.122 | 1.00 | 76.63 | A |
| ATOM | 472 | CG | PRO A | 73 | 62.924 | 48.937 | 69.852 | 1.00 | 76.34 | A |
| ATOM | 473 | C | PRO A | 73 | 66.099 | 50.325 | 68.511 | 0.00 | 76.16 | A |
| ATOM | 474 | O | PRO A | 73 | 67.292 | 50.365 | 68.813 | 0.00 | 76.37 | A |
| ATOM | 475 | N | VAL A | 86 | 52.596 | 52.678 | 63.351 | 1.00 | 68.06 | A |
| ATOM | 476 | CA | VAL A | 86 | 52.119 | 51.882 | 64.474 | 1.00 | 69.23 | A |
| ATOM | 477 | CB | VAL A | 86 | 53.259 | 50.998 | 65.073 | 1.00 | 69.13 | A |
| ATOM | 478 | CG1 | VAL A | 86 | 52.716 | 50.130 | 66.203 | 1.00 | 68.76 | A |
| ATOM | 479 | CG2 | VAL A | 86 | 53.864 | 50.111 | 63.989 | 1.00 | 69.85 | A |
| ATOM | 480 | C | VAL A | 86 | 51.576 | 52.809 | 65.562 | 1.00 | 69.62 | A |
| ATOM | 481 | O | VAL A | 86 | 50.459 | 52.627 | 66.061 | 1.00 | 69.53 | A |
| ATOM | 482 | N | ASP A | 87 | 52.368 | 53.821 | 65.907 | 1.00 | 69.73 | A |
| ATOM | 483 | CA | ASP A | 87 | 51.995 | 54.792 | 66.936 | 1.00 | 68.97 | A |
| ATOM | 484 | CB | ASP A | 87 | 53.177 | 55.726 | 67.223 | 1.00 | 70.05 | A |
| ATOM | 485 | CG | ASP A | 87 | 52.982 | 56.555 | 68.487 | 1.00 | 70.55 | A |
| ATOM | 486 | OD1 | ASP A | 87 | 52.047 | 57.386 | 68.514 | 1.00 | 70.97 | A |
| ATOM | 487 | OD2 | ASP A | 87 | 53.768 | 56.374 | 69.447 | 1.00 | 69.47 | A |
| ATOM | 488 | C | ASP A | 87 | 50.766 | 55.609 | 66.528 | 1.00 | 67.85 | A |
| ATOM | 489 | O | ASP A | 87 | 50.574 | 55.925 | 65.353 | 1.00 | 67.51 | A |
| ATOM | 490 | N | SER A | 88 | 49.936 | 55.948 | 67.510 | 1.00 | 66.47 | A |
| ATOM | 491 | CA | SER A | 88 | 48.714 | 56.708 | 67.263 | 1.00 | 64.69 | A |
| ATOM | 492 | CB | SER A | 88 | 47.872 | 56.772 | 68.537 | 1.00 | 65.29 | A |
| ATOM | 493 | OG | SER A | 88 | 46.724 | 57.579 | 68.337 | 1.00 | 66.37 | A |
| ATOM | 494 | C | SER A | 88 | 48.976 | 58.124 | 66.770 | 1.00 | 62.66 | A |
| ATOM | 495 | O | SER A | 88 | 48.420 | 58.553 | 65.755 | 1.00 | 61.70 | A |
| ATOM | 496 | N | TRP A | 89 | 49.824 | 58.843 | 67.498 | 1.00 | 60.57 | A |
| ATOM | 497 | CA | TRP A | 89 | 50.161 | 60.221 | 67.160 | 1.00 | 58.68 | A |
| ATOM | 498 | CB | TRP A | 89 | 50.999 | 60.836 | 68.281 | 1.00 | 57.33 | A |
| ATOM | 499 | CG | TRP A | 89 | 51.130 | 62.296 | 68.148 | 1.00 | 56.37 | A |
| ATOM | 500 | CD2 | TRP A | 89 | 52.229 | 62.998 | 67.571 | 1.00 | 56.15 | A |
| ATOM | 501 | CE2 | TRP A | 89 | 51.892 | 64.365 | 67.574 | 1.00 | 55.64 | A |
| ATOM | 502 | CE3 | TRP A | 89 | 53.463 | 62.605 | 67.044 | 1.00 | 55.77 | A |
| ATOM | 503 | CD1 | TRP A | 89 | 50.201 | 63.231 | 68.477 | 1.00 | 54.82 | A |
| ATOM | 504 | NE1 | TRP A | 89 | 50.649 | 64.480 | 68.136 | 1.00 | 54.93 | A |
| ATOM | 505 | CZ2 | TRP A | 89 | 52.755 | 65.342 | 67.072 | 1.00 | 55.82 | A |
| ATOM | 506 | CZ3 | TRP A | 89 | 54.315 | 63.579 | 66.544 | 1.00 | 55.23 | A |
| ATOM | 507 | CH2 | TRP A | 89 | 53.957 | 64.927 | 66.561 | 1.00 | 55.54 | A |
| ATOM | 508 | C | TRP A | 89 | 50.915 | 60.332 | 65.831 | 1.00 | 57.32 | A |
| ATOM | 509 | O | TRP A | 89 | 50.679 | 61.248 | 65.045 | 1.00 | 57.65 | A |
| ATOM | 510 | NA | SP A | 90 | 51.827 | 59.398 | 65.593 | 1.00 | 55.46 | A |
| ATOM | 511 | CA | ASP A | 90 | 52.602 | 59.387 | 64.366 | 1.00 | 53.21 | A |
| ATOM | 512 | CB | ASP A | 90 | 53.710 | 58.353 | 64.472 | 1.00 | 51.45 | A |
| ATOM | 513 | CG | ASP A | 90 | 54.795 | 58.795 | 65.396 | 1.00 | 51.44 | A |
| ATOM | 514 | OD1 | ASP A | 90 | 54.462 | 59.439 | 66.401 | 1.00 | 53.33 | A |
| ATOM | 515 | OD2 | ASP A | 90 | 55.975 | 58.511 | 65.134 | 1.00 | 51.16 | A |
| ATOM | 516 | C | ASP A | 90 | 51.731 | 59.082 | 63.162 | 1.00 | 53.01 | A |
| ATOM | 517 | O | ASP A | 90 | 51.943 | 59.625 | 62.076 | 1.00 | 53.15 | A |
| ATOM | 518 | N | ARG A | 91 | 50.746 | 58.214 | 63.362 | 1.00 | 52.99 | A |
| ATOM | 519 | CA | ARG A | 91 | 49.835 | 57.822 | 62.293 | 1.00 | 52.82 | A |
| ATOM | 520 | CB | ARG A | 91 | 48.865 | 56.747 | 62.802 | 1.00 | 53.81 | A |
| ATOM | 521 | CG | ARG A | 91 | 47.856 | 56.264 | 61.770 | 1.00 | 56.18 | A |
| ATOM | 522 | CD | ARG A | 91 | 47.412 | 54.828 | 62.031 | 1.00 | 58.29 | A |
| ATOM | 523 | NE | ARG A | 91 | 46.995 | 54.617 | 63.412 | 1.00 | 61.35 | A |
| ATOM | 524 | CZ | ARG A | 91 | 45.922 | 55.168 | 63.972 | 1.00 | 62.36 | A |
| ATOM | 525 | NH1 | ARG A | 91 | 45.138 | 55.972 | 63.267 | 1.00 | 64.03 | A |
| ATOM | 526 | NH2 | ARG A | 91 | 45.640 | 54.925 | 65.246 | 1.00 | 63.29 | A |
| ATOM | 527 | C | ARG A | 91 | 49.065 | 59.014 | 61.728 | 1.00 | 51.91 | A |
| ATOM | 528 | O | ARG A | 91 | 48.829 | 59.086 | 60.522 | 1.00 | 51.24 | A |
| ATOM | 529 | N | GLU A | 92 | 48.683 | 59.946 | 62.599 | 1.00 | 50.83 | A |
| ATOM | 530 | CA | GLU A | 92 | 47.955 | 61.136 | 62.172 | 1.00 | 50.40 | A |
| ATOM | 531 | CB | GLU A | 92 | 47.181 | 61.750 | 63.337 | 1.00 | 52.44 | A |
| ATOM | 532 | CG | GLU A | 92 | 46.026 | 60.914 | 63.856 | 1.00 | 58.05 | A |
| ATOM | 533 | CD | GLU A | 92 | 44.989 | 60.594 | 62.787 | 1.00 | 60.95 | A |
| ATOM | 534 | OE1 | GLU A | 92 | 44.493 | 61.541 | 62.125 | 1.00 | 61.85 | A |
| ATOM | 535 | OE2 | GLU A | 92 | 44.670 | 59.392 | 62.621 | 1.00 | 61.47 | A |
| ATOM | 536 | C | GLU A | 92 | 48.929 | 62.174 | 61.632 | 1.00 | 48.17 | A |
| ATOM | 537 | O | GLU A | 92 | 48.689 | 62.784 | 60.589 | 1.00 | 48.86 | A |
| ATOM | 538 | N | PHE A | 93 | 50.020 | 62.376 | 62.364 | 1.00 | 45.11 | A |
| ATOM | 539 | CA | PHE A | 93 | 51.056 | 63.325 | 61.989 | 1.00 | 42.22 | A |
| ATOM | 540 | CB | PHE A | 93 | 52.241 | 63.197 | 62.930 | 1.00 | 39.86 | A |
| ATOM | 541 | CG | PHE A | 93 | 53.473 | 63.881 | 62.431 | 1.00 | 38.61 | A |
| ATOM | 542 | CD1 | PHE A | 93 | 53.560 | 65.270 | 62.434 | 1.00 | 38.78 | A |
| ATOM | 543 | CD2 | PHE A | 93 | 54.550 | 63.139 | 61.949 | 1.00 | 38.41 | A |
| ATOM | 544 | CE1 | PHE A | 93 | 54.706 | 65.920 | 61.962 | 1.00 | 37.98 | A |
| ATOM | 545 | CE2 | PHE A | 93 | 55.702 | 63.771 | 61.474 | 1.00 | 37.53 | A |
| ATOM | 546 | CZ | PHE A | 93 | 55.782 | 65.169 | 61.482 | 1.00 | 37.66 | A |
| ATOM | 547 | C | PHE A | 93 | 51.529 | 63.039 | 60.586 | 1.00 | 41.92 | A |
| ATOM | 548 | O | PHE A | 93 | 51.915 | 63.940 | 59.847 | 1.00 | 42.62 | A |
| ATOM | 549 | N | LEU A | 94 | 51.513 | 61.766 | 60.229 | 1.00 | 41.18 | A |
| ATOM | 550 | CA | LEU A | 94 | 51.938 | 61.368 | 58.913 | 1.00 | 40.65 | A |
| ATOM | 551 | CB | LEU A | 94 | 52.602 | 60.008 | 58.986 | 1.00 | 39.81 | A |
| ATOM | 552 | CG | LEU A | 94 | 53.987 | 60.082 | 59.611 | 1.00 | 40.12 | A |
| ATOM | 553 | CD1 | LEU A | 94 | 54.560 | 58.682 | 59.692 | 1.00 | 40.00 | A |
| ATOM | 554 | CD2 | LEU A | 94 | 54.889 | 60.998 | 58.785 | 1.00 | 38.29 | A |
| ATOM | 555 | C | LEU A | 94 | 50.792 | 61.333 | 57.927 | 1.00 | 41.17 | A |
| ATOM | 556 | O | LEU A | 94 | 51.010 | 61.120 | 56.737 | 1.00 | 41.27 | A |
| ATOM | 557 | N | LYS A | 95 | 49.575 | 61.553 | 58.413 | 1.00 | 41.25 | A |
| ATOM | 558 | CA | LYS A | 95 | 48.420 | 61.534 | 57.532 | 1.00 | 42.22 | A |
| ATOM | 559 | CB | LYS A | 95 | 47.120 | 61.460 | 58.342 | 1.00 | 43.14 | A |
| ATOM | 560 | CG | LYS A | 95 | 45.925 | 61.168 | 57.438 | 1.00 | 47.48 | A |
| ATOM | 561 | CD | LYS A | 95 | 44.586 | 61.331 | 58.138 | 1.00 | 51.30 | A |
| ATOM | 562 | CE | LYS A | 95 | 44.392 | 60.305 | 59.252 | 1.00 | 53.68 | A |
| ATOM | 563 | NZ | LYS A | 95 | 43.042 | 60.431 | 59.895 | 1.00 | 54.53 | A |
| ATOM | 564 | C | LYS A | 95 | 48.390 | 62.762 | 56.616 | 1.00 | 41.83 | A |
| ATOM | 565 | O | LYS A | 95 | 47.450 | 63.552 | 56.650 | 1.00 | 42.87 | A |
| ATOM | 566 | N | VAL A | 96 | 49.406 | 62.890 | 55.770 | 1.00 | 41.34 | A |
| ATOM | 567 | CA | VAL A | 96 | 49.545 | 64.023 | 54.856 | 1.00 | 40.71 | A |
| ATOM | 568 | CB | VAL A | 96 | 50.909 | 64.673 | 55.057 | 1.00 | 40.36 | A |
| ATOM | 569 | CG1 | VAL A | 96 | 51.034 | 65.180 | 56.471 | 1.00 | 39.98 | A |
| ATOM | 570 | CG2 | VAL A | 96 | 51.990 | 63.651 | 54.777 | 1.00 | 38.65 | A |
| ATOM | 571 | C | VAL A | 96 | 49.446 | 63.611 | 53.385 | 1.00 | 41.10 | A |
| ATOM | 572 | O | VAL A | 96 | 49.074 | 62.482 | 53.077 | 1.00 | 39.99 | A |
| ATOM | 573 | N | ASP A | 97 | 49.789 | 64.518 | 52.471 | 1.00 | 41.74 | A |
| ATOM | 574 | CA | ASP A | 97 | 49.729 | 64.155 | 51.064 | 1.00 | 43.35 | A |
| ATOM | 575 | CB | ASP A | 97 | 49.500 | 65.386 | 50.168 | 1.00 | 46.03 | A |
| ATOM | 576 | CG | ASP A | 97 | 50.694 | 66.307 | 50.088 | 1.00 | 49.27 | A |
| ATOM | 577 | OD1 | ASP A | 97 | 51.762 | 65.862 | 49.621 | 1.00 | 51.92 | A |
| ATOM | 578 | OD2 | ASP A | 97 | 50.560 | 67.490 | 50.476 | 1.00 | 50.75 | A |
| ATOM | 579 | C | ASP A | 97 | 50.976 | 63.369 | 50.642 | 1.00 | 42.71 | A |
| ATOM | 580 | O | ASP A | 97 | 52.039 | 63.493 | 51.249 | 1.00 | 42.39 | A |
| ATOM | 581 | N | GLN A | 98 | 50.815 | 62.549 | 49.606 | 1.00 | 41.92 | A |
| ATOM | 582 | CA | GLN A | 98 | 51.867 | 61.690 | 49.097 | 1.00 | 40.58 | A |
| ATOM | 583 | CB | GLN A | 98 | 51.392 | 60.988 | 47.807 | 1.00 | 42.66 | A |
| ATOM | 584 | CG | GLN A | 98 | 49.949 | 60.414 | 47.882 | 1.00 | 43.12 | A |
| ATOM | 585 | CD | GLN A | 98 | 49.666 | 59.270 | 46.905 | 1.00 | 43.62 | A |
| ATOM | 586 | OE1 | CLN A | 98 | 48.516 | 58.876 | 46.720 | 1.00 | 42.97 | A |
| ATOM | 587 | NE2 | GLN A | 98 | 50.712 | 58.732 | 46.290 | 1.00 | 44.27 | A |
| ATOM | 588 | C | GLN A | 98 | 53.173 | 62.428 | 48.870 | 1.00 | 39.89 | A |
| ATOM | 589 | O | GLN A | 98 | 54.234 | 61.905 | 49.180 | 1.00 | 38.30 | A |
| ATOM | 590 | N | GLU A | 99 | 53.114 | 63.640 | 48.330 | 1.00 | 40.88 | A |
| ATOM | 591 | CA | GLU A | 99 | 54.343 | 64.404 | 48.118 | 1.00 | 40.74 | A |
| ATOM | 592 | CB | GLU A | 99 | 54.035 | 65.695 | 47.377 | 1.00 | 41.69 | A |
| ATOM | 593 | CG | GLU A | 99 | 53.880 | 65.494 | 45.903 | 1.00 | 47.00 | A |
| ATOM | 594 | CD | GLU A | 99 | 55.182 | 65.074 | 45.254 | 1.00 | 48.98 | A |
| ATOM | 595 | OE1 | GLU A | 99 | 56.236 | 65.569 | 45.721 | 1.00 | 50.30 | A |
| ATOM | 596 | OE2 | GLU A | 99 | 55.154 | 64.274 | 44.284 | 1.00 | 48.68 | A |
| ATOM | 597 | C | GLU A | 99 | 54.984 | 64.710 | 49.474 | 1.00 | 39.87 | A |
| ATOM | 598 | O | GLU A | 99 | 56.164 | 64.426 | 49.691 | 1.00 | 39.94 | A |
| ATOM | 599 | N | MSE A | 100 | 54.187 | 65.272 | 50.382 | 1.00 | 39.05 | A |
| ATOM | 600 | CA | MSE A | 100 | 54.637 | 65.617 | 51.729 | 1.00 | 39.03 | A |
| ATOM | 601 | CB | MSE A | 100 | 53.434 | 66.000 | 52.603 | 1.00 | 45.07 | A |
| ATOM | 602 | CG | MSE A | 100 | 53.726 | 66.188 | 54.093 | 1.00 | 50.78 | A |
| ATOM | 603 | SE | MSE A | 100 | 54.480 | 67.900 | 54.500 | 1.00 | 61.41 | A |
| ATOM | 604 | CE | MSE A | 100 | 56.351 | 67.467 | 54.330 | 1.00 | 55.82 | A |
| ATOM | 605 | C | MSE A | 100 | 55.366 | 64.446 | 52.371 | 1.00 | 36.07 | A |
| ATOM | 606 | O | MSE A | 100 | 56.517 | 64.561 | 52.789 | 1.00 | 34.98 | A |
| ATOM | 607 | N | LEU A | 101 | 54.681 | 63.316 | 52.436 | 1.00 | 32.38 | A |
| ATOM | 608 | CA | LEU A | 101 | 55.238 | 62.126 | 53.031 | 1.00 | 31.39 | A |
| ATOM | 609 | CB | LEU A | 101 | 54.241 | 60.993 | 52.880 | 1.00 | 30.22 | A |
| ATOM | 610 | CG | LEU A | 101 | 54.616 | 59.676 | 53.538 | 1.00 | 29.99 | A |
| ATOM | 611 | CD1 | LEU A | 101 | 54.859 | 59.906 | 55.028 | 1.00 | 29.67 | A |
| ATOM | 612 | CD2 | LEU A | 101 | 53.501 | 58.674 | 53.287 | 1.00 | 26.62 | A |
| ATOM | 613 | C | LEU A | 101 | 56.571 | 61.745 | 52.394 | 1.00 | 31.59 | A |
| ATOM | 614 | O | LEU A | 101 | 57.523 | 61.341 | 53.077 | 1.00 | 32.03 | A |
| ATOM | 615 | N | TYR A | 102 | 56.624 | 61.872 | 51.076 | 1.00 | 30.32 | A |
| ATOM | 616 | CA | TYR A | 102 | 57.814 | 61.556 | 50.311 | 1.00 | 29.70 | A |
| ATOM | 617 | CB | TYR A | 102 | 57.550 | 61.840 | 48.838 | 1.00 | 29.09 | A |
| ATOM | 618 | CG | TYR A | 102 | 58.765 | 61.747 | 47.940 | 1.00 | 27.92 | A |
| ATOM | 619 | CD1 | TYR A | 102 | 59.402 | 60.530 | 47.709 | 1.00 | 27.22 | A |
| ATOM | 620 | CE1 | TYR A | 102 | 60.478 | 60.441 | 46.831 | 1.00 | 27.22 | A |
| ATOM | 621 | CD2 | TYR A | 102 | 59.243 | 62.873 | 47.274 | 1.00 | 26.92 | A |
| ATOM | 622 | CE2 | TYR A | 102 | 60.314 | 62.792 | 46.401 | 1.00 | 24.90 | A |
| ATOM | 623 | CZ | TYR A | 102 | 60.921 | 61.580 | 46.181 | 1.00 | 26.49 | A |
| ATOM | 624 | OH | TYR A | 102 | 61.959 | 61.497 | 45.299 | 1.00 | 26.37 | A |
| ATOM | 625 | C | TYR A | 102 | 58.970 | 62.409 | 50.796 | 1.00 | 30.50 | A |
| ATOM | 626 | O | TYR A | 102 | 60.057 | 61.905 | 51.125 | 1.00 | 30.28 | A |
| ATOM | 627 | N | GLU A | 103 | 58.731 | 63.715 | 50.839 | 1.00 | 31.34 | A |
| ATOM | 628 | CA | GLU A | 103 | 59.763 | 64.645 | 51.276 | 1.00 | 32.16 | A |
| ATOM | 629 | CB | GLU A | 103 | 59.314 | 66.088 | 50.966 | 1.00 | 32.79 | A |
| ATOM | 630 | CG | GLU A | 103 | 58.921 | 66.251 | 49.492 | 1.00 | 35.11 | A |
| ATOM | 631 | CD | GLU A | 103 | 58.538 | 67.673 | 49.065 | 1.00 | 37.94 | A |
| ATOM | 632 | OE1 | GLU A | 103 | 57.628 | 68.279 | 49.685 | 1.00 | 37.38 | A |
| ATOM | 633 | OE2 | GLU A | 103 | 59.138 | 68.175 | 48.084 | 1.00 | 37.07 | A |
| ATOM | 634 | C | GLU A | 103 | 60.128 | 64.438 | 52.760 | 1.00 | 30.98 | A |
| ATOM | 635 | O | GLU A | 103 | 61.292 | 64.600 | 53.136 | 1.00 | 30.87 | A |
| ATOM | 636 | N | ILE A | 104 | 59.159 | 64.038 | 53.588 | 1.00 | 29.27 | A |
| ATOM | 637 | CA | ILE A | 104 | 59.426 | 63.804 | 55.011 | 1.00 | 28.70 | A |
| ATOM | 638 | CB | ILE A | 104 | 58.113 | 63.589 | 55.813 | 1.00 | 28.55 | A |
| ATOM | 639 | CG2 | ILE A | 104 | 58.424 | 62.958 | 57.189 | 1.00 | 29.44 | A |
| ATOM | 640 | CG1 | ILE A | 104 | 57.396 | 64.942 | 55.975 | 1.00 | 26.70 | A |
| ATOM | 641 | CD1 | ILE A | 104 | 56.098 | 64.917 | 56.782 | 1.00 | 22.17 | A |
| ATOM | 642 | C | ILE A | 104 | 60.359 | 62.609 | 55.221 | 1.00 | 27.96 | A |
| ATOM | 643 | O | ILE A | 104 | 61.180 | 62.591 | 56.144 | 1.00 | 26.87 | A |
| ATOM | 644 | N | ILE A | 105 | 60.225 | 61.609 | 54.360 | 1.00 | 27.92 | A |
| ATOM | 645 | CA | ILE A | 105 | 61.082 | 60.439 | 54.435 | 1.00 | 26.71 | A |
| ATOM | 646 | CB | ILE A | 105 | 60.543 | 59.321 | 53.534 | 1.00 | 26.61 | A |
| ATOM | 647 | CG2 | ILE A | 105 | 61.590 | 58.215 | 53.388 | 1.00 | 25.95 | A |
| ATOM | 648 | CG1 | ILE A | 105 | 59.200 | 58.836 | 54.106 | 1.00 | 26.49 | A |
| ATOM | 649 | CD1 | ILE A | 105 | 58.546 | 57.701 | 53.349 | 1.00 | 26.37 | A |
| ATOM | 650 | C | ILE A | 105 | 62.500 | 60.825 | 54.017 | 1.00 | 26.46 | A |
| ATOM | 651 | O | ILE A | 105 | 63.484 | 60.401 | 54.639 | 1.00 | 26.10 | A |
| ATOM | 652 | N | LEU A | 106 | 62.603 | 61.648 | 52.975 | 1.00 | 25.01 | A |
| ATOM | 653 | CA | LEU A | 106 | 63.915 | 62.084 | 52.505 | 1.00 | 24.17 | A |
| ATOM | 654 | CB | LEU A | 106 | 63.802 | 62.917 | 51.230 | 1.00 | 23.30 | A |
| ATOM | 655 | CG | LEU A | 106 | 63.211 | 62.283 | 49.961 | 1.00 | 22.86 | A |
| ATOM | 656 | CD1 | LEU A | 106 | 63.415 | 63.252 | 48.829 | 1.00 | 19.46 | A |
| ATOM | 657 | CD2 | LEU A | 106 | 63.875 | 60.950 | 49.639 | 1.00 | 17.99 | A |
| ATOM | 658 | C | LEU A | 106 | 64.612 | 62.904 | 53.574 | 1.00 | 24.05 | A |
| ATOM | 659 | O | LEU A | 106 | 65.792 | 62.701 | 53.860 | 1.00 | 24.80 | A |
| ATOM | 660 | N | ALA A | 107 | 63.875 | 63.835 | 54.168 | 1.00 | 23.58 | A |
| ATOM | 661 | CA | ALA A | 107 | 64.430 | 64.676 | 55.218 | 1.00 | 23.64 | A |
| ATOM | 662 | CB | ALA A | 107 | 63.388 | 65.684 | 55.684 | 1.00 | 21.11 | A |
| ATOM | 663 | C | ALA A | 107 | 64.901 | 63.817 | 56.393 | 1.00 | 23.90 | A |
| ATOM | 664 | O | ALA A | 107 | 65.978 | 64.046 | 56.952 | 1.00 | 23.39 | A |
| ATOM | 665 | N | ALA A | 108 | 64.093 | 62.838 | 56.779 | 1.00 | 24.83 | A |
| ATOM | 666 | CA | ALA A | 108 | 64.479 | 61.970 | 57.879 | 1.00 | 27.43 | A |
| ATOM | 667 | CB | ALA A | 108 | 63.343 | 60.984 | 58.215 | 1.00 | 27.65 | A |
| ATOM | 668 | C | ALA A | 108 | 65.727 | 61.211 | 57.456 | 1.00 | 28.80 | A |
| ATOM | 669 | O | ALA A | 108 | 66.646 | 61.009 | 58.249 | 1.00 | 29.92 | A |
| ATOM | 670 | N | ASN A | 109 | 65.763 | 60.794 | 56.196 | 1.00 | 28.89 | A |
| ATOM | 671 | CA | ASN A | 109 | 66.920 | 60.062 | 55.719 | 1.00 | 29.68 | A |
| ATOM | 672 | CB | ASN A | 109 | 66.631 | 59.416 | 54.361 | 1.00 | 31.27 | A |
| ATOM | 673 | CG | ASN A | 109 | 67.744 | 58.480 | 53.915 | 1.00 | 31.80 | A |
| ATOM | 674 | OD1 | ASN A | 109 | 68.755 | 58.912 | 53.362 | 1.00 | 31.60 | A |
| ATOM | 675 | ND2 | ASN A | 109 | 67.568 | 57.186 | 54.178 | 1.00 | 32.93 | A |
| ATOM | 676 | C | ASN A | 109 | 68.153 | 60.956 | 55.628 | 1.00 | 30.29 | A |
| ATOM | 677 | O | ASN A | 109 | 69.248 | 60.524 | 55.959 | 1.00 | 30.81 | A |
| ATOM | 678 | N | TYR A | 110 | 67.992 | 62.197 | 55.177 | 1.00 | 31.42 | A |
| ATOM | 679 | CA | TYR A | 110 | 69.133 | 63.105 | 55.080 | 1.00 | 31.10 | A |
| ATOM | 680 | CB | TYR A | 110 | 68.756 | 64.363 | 54.312 | 1.00 | 29.98 | A |
| ATOM | 681 | CG | TYR A | 110 | 69.819 | 65.437 | 54.351 | 1.00 | 30.69 | A |
| ATOM | 682 | CD1 | TYR A | 110 | 71.057 | 65.237 | 53.736 | 1.00 | 29.71 | A |
| ATOM | 683 | CE1 | TYR A | 110 | 72.022 | 66.241 | 53.716 | 1.00 | 30.17 | A |
| ATOM | 684 | CD2 | TYR A | 110 | 69.571 | 66.674 | 54.965 | 1.00 | 31.52 | A |
| ATOM | 685 | CE2 | TYR A | 110 | 70.526 | 67.686 | 54.954 | 1.00 | 31.71 | A |
| ATOM | 686 | CZ | TYR A | 110 | 71.750 | 67.463 | 54.318 | 1.00 | 32.53 | A |
| ATOM | 687 | OH | TYR A | 110 | 72.680 | 68.481 | 54.226 | 1.00 | 33.63 | A |
| ATOM | 688 | C | TYR A | 110 | 69.620 | 63.515 | 56.463 | 1.00 | 31.73 | A |
| ATOM | 689 | O | TYR A | 110 | 70.818 | 63.603 | 56.694 | 1.00 | 32.81 | A |
| ATOM | 690 | N | LEU A | 111 | 68.682 | 63.770 | 57.371 | 1.00 | 30.92 | A |
| ATOM | 691 | CA | LEU A | 111 | 69.016 | 64.184 | 58.719 | 1.00 | 31.21 | A |
| ATOM | 692 | CB | LEU A | 111 | 67.870 | 64.994 | 59.317 | 1.00 | 28.89 | A |
| ATOM | 693 | CG | LEU A | 111 | 67.630 | 66.380 | 58.734 | 1.00 | 26.61 | A |
| ATOM | 694 | CD1 | LEU A | 111 | 66.251 | 66.831 | 59.141 | 1.00 | 24.78 | A |
| ATOM | 695 | CD2 | LEU A | 111 | 68.694 | 67.360 | 59.200 | 1.00 | 24.10 | A |
| ATOM | 696 | C | LEU A | 111 | 69.337 | 63.006 | 59.624 | 1.00 | 33.12 | A |
| ATOM | 697 | O | LEU A | 111 | 69.590 | 63.181 | 60.816 | 1.00 | 34.02 | A |
| ATOM | 698 | N | ASN A | 112 | 69.319 | 61.805 | 59.062 | 1.00 | 33.71 | A |
| ATOM | 699 | CA | ASN A | 112 | 69.620 | 60.588 | 59.820 | 1.00 | 34.83 | A |
| ATOM | 700 | CB | ASN A | 112 | 71.102 | 60.570 | 60.204 | 1.00 | 33.95 | A |
| ATOM | 701 | CG | ASN A | 112 | 71.644 | 59.164 | 60.365 | 1.00 | 33.26 | A |
| ATOM | 702 | OD1 | ASN A | 112 | 71.015 | 58.322 | 60.977 | 1.00 | 34.03 | A |
| ATOM | 703 | ND2 | ASN A | 112 | 72.820 | 58.913 | 59.813 | 1.00 | 34.72 | A |
| ATOM | 704 | C | ASN A | 112 | 68.764 | 60.460 | 61.091 | 1.00 | 35.67 | A |
| ATOM | 705 | O | ASN A | 112 | 69.272 | 60.582 | 62.209 | 1.00 | 37.75 | A |
| ATOM | 706 | N | ILE A | 113 | 67.472 | 60.203 | 60.906 | 1.00 | 35.48 | A |
| ATOM | 707 | CA | ILE A | 113 | 66.517 | 60.055 | 62.005 | 1.00 | 35.39 | A |
| ATOM | 708 | CB | ILE A | 113 | 65.531 | 61.253 | 62.034 | 1.00 | 35.97 | A |
| ATOM | 709 | CG2 | ILE A | 113 | 64.563 | 61.125 | 63.200 | 1.00 | 35.08 | A |
| ATOM | 710 | CG1 | ILE A | 113 | 66.307 | 62.567 | 62.154 | 1.00 | 36.19 | A |
| ATOM | 711 | CD1 | ILE A | 113 | 65.427 | 63.795 | 62.132 | 1.00 | 33.68 | A |
| ATOM | 712 | C | ILE A | 113 | 65.738 | 58.761 | 61.763 | 1.00 | 35.67 | A |
| ATOM | 713 | O | ILE A | 113 | 64.564 | 58.789 | 61.381 | 1.00 | 33.98 | A |
| ATOM | 714 | N | LYS A | 114 | 66.408 | 57.632 | 61.988 | 1.00 | 37.77 | A |
| ATOM | 715 | CA | LYS A | 114 | 65.827 | 56.299 | 61.775 | 1.00 | 39.09 | A |
| ATOM | 716 | CB | LYS A | 114 | 66.697 | 55.238 | 62.461 | 1.00 | 41.82 | A |
| ATOM | 717 | CG | LYS A | 114 | 67.835 | 54.703 | 61.601 | 1.00 | 45.96 | A |
| ATOM | 718 | CD | LYS A | 114 | 68.786 | 55.789 | 61.081 | 1.00 | 47.81 | A |
| ATOM | 719 | CE | LYS A | 114 | 69.952 | 55.145 | 60.306 | 1.00 | 49.70 | A |
| ATOM | 720 | NZ | LYS A | 114 | 69.498 | 54.249 | 59.161 | 1.00 | 51.87 | A |
| ATOM | 721 | C | LYS A | 114 | 64.372 | 56.128 | 62.216 | 1.00 | 37.68 | A |
| ATOM | 722 | O | LYS A | 114 | 63.521 | 55.697 | 61.438 | 1.00 | 38.17 | A |
| ATOM | 723 | N | PRO A | 115 | 64.076 | 56.435 | 63.484 | 1.00 | 36.13 | A |
| ATOM | 724 | CD | PRO A | 115 | 65.015 | 56.886 | 64.526 | 1.00 | 34.15 | A |
| ATOM | 725 | CA | PRO A | 115 | 62.715 | 56.314 | 64.011 | 1.00 | 34.44 | A |
| ATOM | 726 | CB | PRO A | 115 | 62.815 | 57.022 | 65.351 | 1.00 | 34.06 | A |
| ATOM | 727 | CG | PRO A | 115 | 64.220 | 56.680 | 65.777 | 1.00 | 35.33 | A |
| ATOM | 728 | C | PRO A | 115 | 61.694 | 56.968 | 63.076 | 1.00 | 33.56 | A |
| ATOM | 729 | O | PRO A | 115 | 60.658 | 56.384 | 62.763 | 1.00 | 33.50 | A |
| ATOM | 730 | N | LEU A | 116 | 62.001 | 58.179 | 62.622 | 1.00 | 31.71 | A |
| ATOM | 731 | CA | LEU A | 116 | 61.108 | 58.903 | 61.733 | 1.00 | 30.40 | A |
| ATOM | 732 | CB | LEU A | 116 | 61.583 | 60.347 | 61.567 | 1.00 | 29.42 | A |
| ATOM | 733 | CG | LEU A | 116 | 60.603 | 61.229 | 60.796 | 1.00 | 28.24 | A |
| ATOM | 734 | CD1 | LEU A | 116 | 59.268 | 61.223 | 61.507 | 1.00 | 28.18 | A |
| ATOM | 735 | CD2 | LEU A | 116 | 61.144 | 62.633 | 60.674 | 1.00 | 29.52 | A |
| ATOM | 736 | C | LEU A | 116 | 61.005 | 58.211 | 60.375 | 1.00 | 30.54 | A |
| ATOM | 737 | O | LEU A | 116 | 59.909 | 58.046 | 59.823 | 1.00 | 29.43 | A |
| ATOM | 738 | N | LEU A | 117 | 62.150 | 57.804 | 59.836 | 1.00 | 30.80 | A |
| ATOM | 739 | CA | LEU A | 117 | 62.193 | 57.097 | 58.555 | 1.00 | 30.78 | A |
| ATOM | 740 | CB | LEU A | 117 | 63.632 | 56.678 | 58.223 | 1.00 | 28.60 | A |
| ATOM | 741 | CG | LEU A | 117 | 63.843 | 55.833 | 56.966 | 1.00 | 27.90 | A |
| ATOM | 742 | CD1 | LEU A | 117 | 63.388 | 56.587 | 55.756 | 1.00 | 26.40 | A |
| ATOM | 743 | CD2 | LEU A | 117 | 65.304 | 55.467 | 56.830 | 1.00 | 29.33 | A |
| ATOM | 744 | C | LEU A | 117 | 61.311 | 55.856 | 58.624 | 1.00 | 32.57 | A |
| ATOM | 745 | O | LEU A | 117 | 60.403 | 55.675 | 57.800 | 1.00 | 33.66 | A |
| ATOM | 746 | N | ASP A | 118 | 61.588 | 55.002 | 59.608 | 1.00 | 32.30 | A |
| ATOM | 747 | CA | ASP A | 118 | 60.821 | 53.781 | 59.777 | 1.00 | 32.82 | A |
| ATOM | 748 | CB | ASP A | 118 | 61.241 | 53.035 | 61.046 | 1.00 | 34.51 | A |
| ATOM | 749 | CG | ASP A | 118 | 62.660 | 52.481 | 60.958 | 1.00 | 38.01 | A |
| ATOM | 750 | OD1 | ASP A | 118 | 63.075 | 52.088 | 59.841 | 1.00 | 37.92 | A |
| ATOM | 751 | OD2 | ASP A | 118 | 63.355 | 52.432 | 62.007 | 1.00 | 38.31 | A |
| ATOM | 752 | C | ASP A | 118 | 59.345 | 54.104 | 59.836 | 1.00 | 32.67 | A |
| ATOM | 753 | O | ASP A | 118 | 58.533 | 53.458 | 59.160 | 1.00 | 33.71 | A |
| ATOM | 754 | N | ALA A | 119 | 58.997 | 55.109 | 60.632 | 1.00 | 31.48 | A |
| ATOM | 755 | CA | ALA A | 119 | 57.600 | 55.512 | 60.774 | 1.00 | 32.03 | A |
| ATOM | 756 | CB | ALA A | 119 | 57.512 | 56.753 | 61.625 | 1.00 | 31.77 | A |
| ATOM | 757 | C | ALA A | 119 | 56.961 | 55.774 | 59.418 | 1.00 | 32.00 | A |
| ATOM | 758 | O | ALA A | 119 | 55.932 | 55.185 | 59.071 | 1.00 | 31.09 | A |
| ATOM | 759 | N | GLY A | 120 | 57.582 | 56.673 | 58.660 | 1.00 | 32.93 | A |
| ATOM | 760 | CA | GLY A | 120 | 57.081 | 57.007 | 57.341 | 1.00 | 33.63 | A |
| ATOM | 761 | C | GLY A | 120 | 57.019 | 55.773 | 56.469 | 1.00 | 33.78 | A |
| ATOM | 762 | O | GLY A | 120 | 56.045 | 55.571 | 55.748 | 1.00 | 33.76 | A |
| ATOM | 763 | N | CYS A | 121 | 58.056 | 54.944 | 56.522 | 1.00 | 34.09 | A |
| ATOM | 764 | CA | CYS A | 121 | 58.048 | 53.735 | 55.714 | 1.00 | 37.06 | A |
| ATOM | 765 | CB | CYS A | 121 | 59.370 | 52.985 | 55.852 | 1.00 | 37.59 | A |
| ATOM | 766 | SG | CYS A | 121 | 60.672 | 53.684 | 54.825 | 1.00 | 37.93 | A |
| ATOM | 767 | C | CYS A | 121 | 56.882 | 52.814 | 56.063 | 1.00 | 36.49 | A |
| ATOM | 768 | O | CYS A | 121 | 56.222 | 52.272 | 55.177 | 1.00 | 37.36 | A |
| ATOM | 769 | N | LYS A | 122 | 56.617 | 52.645 | 57.348 | 1.00 | 36.54 | A |
| ATOM | 770 | CA | LYS A | 122 | 55.525 | 51.786 | 57.766 | 1.00 | 37.44 | A |
| ATOM | 771 | CB | LYS A | 122 | 55.445 | 51.751 | 59.288 | 1.00 | 38.22 | A |
| ATOM | 772 | CG | LYS A | 122 | 56.652 | 51.150 | 59.950 | 1.00 | 38.67 | A |
| ATOM | 773 | CD | LYS A | 122 | 56.424 | 51.081 | 61.441 | 1.00 | 42.04 | A |
| ATOM | 774 | CE | LYS A | 122 | 57.663 | 50.592 | 62.185 | 1.00 | 42.25 | A |
| ATOM | 775 | NZ | LYS A | 122 | 57.394 | 50.419 | 63.636 | 1.00 | 41.74 | A |
| ATOM | 776 | C | LYS A | 122 | 54.197 | 52.276 | 57.208 | 1.00 | 36.82 | A |
| ATOM | 777 | O | LYS A | 122 | 53.340 | 51.479 | 56.812 | 1.00 | 36.50 | A |
| ATOM | 778 | N | VAL A | 123 | 54.035 | 53.595 | 57.187 | 1.00 | 36.30 | A |
| ATOM | 779 | CA | VAL A | 123 | 52.810 | 54.208 | 56.699 | 1.00 | 35.72 | A |
| ATOM | 780 | CB | VAL A | 123 | 52.832 | 55.718 | 56.967 | 1.00 | 36.26 | A |
| ATOM | 781 | CG1 | VAL A | 123 | 51.483 | 56.319 | 56.621 | 1.00 | 36.90 | A |
| ATOM | 782 | CG2 | VAL A | 123 | 53.170 | 55.966 | 58.429 | 1.00 | 35.63 | A |
| ATOM | 783 | C | VAL A | 123 | 52.576 | 53.929 | 55.208 | 1.00 | 35.46 | A |
| ATOM | 784 | O | VAL A | 123 | 51.448 | 53.633 | 54.793 | 1.00 | 33.96 | A |
| ATOM | 785 | N | VAL A | 124 | 53.639 | 54.007 | 54.406 | 1.00 | 35.31 | A |
| ATOM | 786 | CA | VAL A | 124 | 53.510 | 53.737 | 52.970 | 1.00 | 34.89 | A |
| ATOM | 787 | CB | VAL A | 124 | 54.815 | 54.026 | 52.177 | 1.00 | 34.43 | A |
| ATOM | 788 | CG1 | VAL A | 124 | 54.564 | 53.832 | 50.682 | 1.00 | 32.55 | A |
| ATOM | 789 | CG2 | VAL A | 124 | 55.294 | 55.442 | 52.449 | 1.00 | 34.56 | A |
| ATOM | 790 | C | VAL A | 124 | 53.135 | 52.273 | 52.767 | 1.00 | 34.07 | A |
| ATOM | 791 | O | VAL A | 124 | 52.317 | 51.947 | 51.898 | 1.00 | 34.52 | A |
| ATOM | 792 | N | ALA A | 125 | 53.734 | 51.401 | 53.575 | 1.00 | 32.52 | A |
| ATOM | 793 | CA | ALA A | 125 | 53.452 | 49.974 | 53.500 | 1.00 | 33.46 | A |
| ATOM | 794 | CB | ALA A | 125 | 54.294 | 49.221 | 54.496 | 1.00 | 31.49 | A |
| ATOM | 795 | C | ALA A | 125 | 51.975 | 49.757 | 53.805 | 1.00 | 35.59 | A |
| ATOM | 796 | O | ALA A | 125 | 51.283 | 48.987 | 53.127 | 1.00 | 35.62 | A |
| ATOM | 797 | N | GLU A | 126 | 51.495 | 50.462 | 54.823 | 1.00 | 37.35 | A |
| ATOM | 798 | CA | GLU A | 126 | 50.107 | 50.357 | 55.213 | 1.00 | 37.88 | A |
| ATOM | 799 | CB | GLU A | 126 | 49.864 | 51.130 | 56.494 | 1.00 | 39.94 | A |
| ATOM | 800 | CG | GLU A | 126 | 50.589 | 50.522 | 57.658 | 1.00 | 44.44 | A |
| ATOM | 801 | CD | GLU A | 126 | 49.884 | 50.789 | 58.974 | 1.00 | 47.25 | A |
| ATOM | 802 | OE1 | GLU A | 126 | 49.926 | 51.949 | 59.454 | 1.00 | 46.76 | A |
| ATOM | 803 | OE2 | GLU A | 126 | 49.271 | 49.832 | 59.516 | 1.00 | 49.61 | A |
| ATOM | 804 | C | GLU A | 126 | 49.149 | 50.812 | 54.142 | 1.00 | 37.12 | A |
| ATOM | 805 | O | GLU A | 126 | 47.974 | 50.472 | 54.189 | 1.00 | 36.85 | A |
| ATOM | 806 | N | MSE A | 127 | 49.648 | 51.573 | 53.176 | 1.00 | 37.83 | A |
| ATOM | 807 | CA | MSE A | 127 | 48.814 | 52.035 | 52.085 | 1.00 | 38.50 | A |
| ATOM | 808 | CB | MSE A | 127 | 49.473 | 53.225 | 51.396 | 1.00 | 39.64 | A |
| ATOM | 809 | CG | MSE A | 127 | 49.682 | 54.471 | 52.264 | 1.00 | 40.86 | A |
| ATOM | 810 | SE | MSE A | 127 | 50.428 | 55.994 | 51.160 | 1.00 | 44.01 | A |
| ATOM | 811 | CE | MSE A | 127 | 48.774 | 56.477 | 50.261 | 1.00 | 43.47 | A |
| ATOM | 812 | C | MSE A | 127 | 48.621 | 50.885 | 51.092 | 1.00 | 38.76 | A |
| ATOM | 813 | O | MSE A | 127 | 47.586 | 50.792 | 50.443 | 1.00 | 38.95 | A |
| ATOM | 814 | N | ILE A | 128 | 49.607 | 49.997 | 51.003 | 1.00 | 39.74 | A |
| ATOM | 815 | CA | ILE A | 128 | 49.552 | 48.849 | 50.090 | 1.00 | 40.70 | A |
| ATOM | 816 | CB | ILE A | 128 | 50.989 | 48.359 | 49.755 | 1.00 | 39.21 | A |
| ATOM | 817 | CG2 | ILE A | 128 | 50.935 | 47.158 | 48.828 | 1.00 | 39.17 | A |
| ATOM | 818 | CG1 | ILE A | 128 | 51.797 | 49.518 | 49.163 | 1.00 | 37.59 | A |
| ATOM | 819 | CD1 | ILE A | 128 | 53.148 | 49.159 | 48.595 | 1.00 | 35.03 | A |
| ATOM | 820 | C | ILE A | 128 | 48.777 | 47.698 | 50.736 | 1.00 | 42.24 | A |
| ATOM | 821 | O | ILE A | 128 | 47.886 | 47.098 | 50.132 | 1.00 | 40.92 | A |
| ATOM | 822 | N | ARG A | 129 | 49.147 | 47.412 | 51.980 | 1.00 | 46.16 | A |
| ATOM | 823 | CA | ARG A | 129 | 48.556 | 46.350 | 52.792 | 1.00 | 49.18 | A |
| ATOM | 824 | CB | ARG A | 129 | 48.696 | 46.701 | 54.268 | 1.00 | 50.29 | A |
| ATOM | 825 | CG | ARG A | 129 | 48.224 | 45.614 | 55.205 | 1.00 | 53.92 | A |
| ATOM | 826 | CD | ARG A | 129 | 48.566 | 45.978 | 56.643 | 1.00 | 58.68 | A |
| ATOM | 827 | NE | ARG A | 129 | 49.936 | 46.490 | 56.743 | 1.00 | 62.87 | A |
| ATOM | 828 | CZ | ARG A | 129 | 50.553 | 46.798 | 57.881 | 1.00 | 64.32 | A |
| ATOM | 829 | NH1 | ARG A | 129 | 49.927 | 46.647 | 59.045 | 1.00 | 64.82 | A |
| ATOM | 830 | NH2 | ARG A | 129 | 51.799 | 47.258 | 57.850 | 1.00 | 64.62 | A |
| ATOM | 831 | C | ARG A | 129 | 47.094 | 46.044 | 52.498 | 1.00 | 49.27 | A |
| ATOM | 832 | O | ARG A | 129 | 46.230 | 46.920 | 52.589 | 1.00 | 49.24 | A |
| ATOM | 833 | N | GLY A | 130 | 46.839 | 44.785 | 52.158 | 1.00 | 48.73 | A |
| ATOM | 834 | CA | GLY A | 130 | 45.492 | 44.354 | 51.873 | 1.00 | 49.33 | A |
| ATOM | 835 | C | GLY A | 130 | 44.843 | 44.937 | 50.632 | 1.00 | 50.07 | A |
| ATOM | 836 | O | GLY A | 130 | 43.619 | 44.856 | 50.486 | 1.00 | 49.22 | A |
| ATOM | 837 | N | ARG A | 131 | 45.630 | 45.523 | 49.735 | 1.00 | 49.98 | A |
| ATOM | 838 | CA | ARG A | 131 | 45.048 | 46.086 | 48.524 | 1.00 | 50.73 | A |
| ATOM | 839 | CB | ARG A | 131 | 45.402 | 47.573 | 48.410 | 1.00 | 51.87 | A |
| ATOM | 840 | CG | ARG A | 131 | 44.801 | 48.429 | 49.545 | 1.00 | 54.75 | A |
| ATOM | 841 | CD | ARG A | 131 | 45.035 | 49.929 | 49.340 | 1.00 | 57.52 | A |
| ATOM | 842 | NE | ARG A | 131 | 44.412 | 50.438 | 48.116 | 1.00 | 60.43 | A |
| ATOM | 843 | CZ | ARG A | 131 | 43.098 | 50.531 | 47.910 | 1.00 | 61.10 | A |
| ATOM | 844 | NH1 | ARG A | 131 | 42.248 | 50.147 | 48.855 | 1.00 | 60.79 | A |
| ATOM | 845 | NH2 | ARG A | 131 | 42.634 | 51.009 | 46.755 | 1.00 | 61.77 | A |
| ATOM | 846 | C | ARG A | 131 | 45.447 | 45.325 | 47.261 | 1.00 | 50.69 | A |
| ATOM | 847 | O | ARG A | 131 | 46.432 | 44.592 | 47.249 | 1.00 | 51.15 | A |
| ATOM | 848 | N | SER A | 132 | 44.670 | 45.494 | 46.199 | 1.00 | 50.35 | A |
| ATOM | 849 | CA | SER A | 132 | 44.941 | 44.798 | 44.950 | 1.00 | 50.32 | A |
| ATOM | 850 | CB | SER A | 132 | 43.630 | 44.489 | 44.224 | 1.00 | 49.61 | A |
| ATOM | 851 | OG | SER A | 132 | 43.082 | 45.662 | 43.648 | 1.00 | 48.59 | A |
| ATOM | 852 | G | SER A | 132 | 45.837 | 45.608 | 44.023 | 1.00 | 50.87 | A |
| ATOM | 853 | O | SER A | 132 | 46.056 | 46.808 | 44.235 | 1.00 | 50.23 | A |
| ATOM | 854 | N | PRO A | 133 | 46.359 | 44.957 | 42.970 | 1.00 | 50.57 | A |
| ATOM | 855 | CD | PRO A | 133 | 46.253 | 43.520 | 42.659 | 1.00 | 49.65 | A |
| ATOM | 856 | CA | PRO A | 133 | 47.230 | 45.628 | 42.006 | 1.00 | 50.93 | A |
| ATOM | 857 | CB | PRO A | 133 | 47.392 | 44.581 | 40.914 | 1.00 | 50.37 | A |
| ATOM | 858 | CG | PRO A | 133 | 47.405 | 43.315 | 41.698 | 1.00 | 50.36 | A |
| ATOM | 859 | C | PRO A | 133 | 46.616 | 46.915 | 41.483 | 1.00 | 51.56 | A |
| ATOM | 860 | O | PRO A | 133 | 47.263 | 47.964 | 41.480 | 1.00 | 52.20 | A |
| ATOM | 861 | N | GLU A | 134 | 45.364 | 46.839 | 41.044 | 1.00 | 51.94 | A |
| ATOM | 862 | CA | GLU A | 134 | 44.693 | 48.021 | 40.516 | 1.00 | 52.22 | A |
| ATOM | 863 | CB | GLU A | 134 | 43.395 | 47.637 | 39.811 | 1.00 | 54.08 | A |
| ATOM | 864 | CG | GLU A | 134 | 43.436 | 47.886 | 38.314 | 1.00 | 58.97 | A |
| ATOM | 865 | CD | GLU A | 134 | 43.821 | 49.320 | 37.974 | 1.00 | 62.39 | A |
| ATOM | 866 | OE1 | GLU A | 134 | 43.122 | 50.254 | 38.436 | 1.00 | 63.15 | A |
| ATOM | 867 | OE2 | GLU A | 134 | 44.823 | 49.513 | 37.243 | 1.00 | 64.67 | A |
| ATOM | 868 | C | GLU A | 134 | 44.406 | 49.035 | 41.610 | 1.00 | 50.15 | A |
| ATOM | 869 | O | GLU A | 134 | 44.400 | 50.244 | 41.355 | 1.00 | 49.58 | A |
| ATOM | 870 | N | GLU A | 135 | 44.177 | 48.539 | 42.823 | 1.00 | 47.26 | A |
| ATOM | 871 | CA | GLU A | 135 | 43.904 | 49.412 | 43.948 | 1.00 | 45.19 | A |
| ATOM | 872 | CB | GLU A | 135 | 43.390 | 48.613 | 45.142 | 1.00 | 44.69 | A |
| ATOM | 873 | CG | GLU A | 135 | 41.973 | 48.135 | 44.948 | 1.00 | 47.13 | A |
| ATOM | 874 | CD | GLU A | 135 | 41.333 | 47.585 | 46.207 | 1.00 | 49.00 | A |
| ATOM | 875 | OE1 | GLU A | 135 | 41.857 | 46.590 | 46.766 | 1.00 | 49.49 | A |
| ATOM | 876 | OE2 | GLU A | 135 | 40.295 | 48.153 | 46.633 | 1.00 | 50.00 | A |
| ATOM | 877 | C | GLU A | 135 | 45.160 | 50.162 | 44.328 | 1.00 | 43.66 | A |
| ATOM | 878 | O | GLU A | 135 | 45.113 | 51.349 | 44.650 | 1.00 | 42.57 | A |
| ATOM | 879 | N | ILE A | 136 | 46.288 | 49.467 | 44.269 | 1.00 | 41.78 | A |
| ATOM | 880 | CA | ILE A | 136 | 47.558 | 50.077 | 44.622 | 1.00 | 41.23 | A |
| ATOM | 881 | CB | ILE A | 136 | 48.646 | 49.014 | 44.782 | 1.00 | 39.22 | A |
| ATOM | 882 | CG2 | ILE A | 136 | 49.957 | 49.675 | 45.197 | 1.00 | 37.75 | A |
| ATOM | 883 | CG1 | ILE A | 136 | 48.180 | 47.970 | 45.801 | 1.00 | 37.01 | A |
| ATOM | 884 | CD1 | ILE A | 136 | 49.154 | 46.847 | 46.033 | 1.00 | 34.37 | A |
| ATOM | 885 | C | ILE A | 136 | 47.993 | 51.065 | 43.558 | 1.00 | 42.50 | A |
| ATOM | 886 | O | ILE A | 136 | 48.490 | 52.162 | 43.856 | 1.00 | 42.93 | A |
| ATOM | 887 | N | ARG A | 137 | 47.787 50.651 | 42.314 | 1.00 | 42.34 | A | |
| ATOM | 888 | CA | ARG A | 137 | 48.144 | 51.439 | 41.151 | 1.00 | 41.82 | A |
| ATOM | 889 | CB | ARG A | 137 | 47.844 | 50.636 | 39.884 | 1.00 | 42.65 | A |
| ATOM | 890 | CG | ARG A | 137 | 48.949 | 50.635 | 38.849 | 1.00 | 43.90 | A |
| ATOM | 891 | CD | ARG A | 137 | 48.594 | 49.715 | 37.679 | 1.00 | 45.63 | A |
| ATOM | 892 | NE | ARG A | 137 | 48.503 | 48.315 | 38.092 | 1.00 | 47.76 | A |
| ATOM | 893 | CZ | ARG A | 137 | 47.567 | 47.477 | 37.664 | 1.00 | 48.21 | A |
| ATOM | 894 | NH1 | ARG A | 137 | 46.644 | 47.901 | 36.810 | 1.00 | 49.10 | A |
| ATOM | 895 | NH2 | ARG A | 137 | 47.546 | 46.226 | 38.099 | 1.00 | 48.10 | A |
| ATOM | 896 | C | ARG A | 137 | 47.348 | 52.736 | 41.156 | 1.00 | 40.67 | A |
| ATOM | 897 | O | ARG A | 137 | 47.821 | 53.773 | 40.685 | 1.00 | 39.93 | A |
| ATOM | 898 | N | ARG A | 138 | 46.134 | 52.677 | 41.694 | 1.00 | 40.67 | A |
| ATOM | 899 | CA | ARG A | 138 | 45.276 | 53.864 | 41.756 | 1.00 | 40.88 | A |
| ATOM | 900 | CB | ARG A | 138 | 43.807 | 53.456 | 41.932 | 1.00 | 39.95 | A |
| ATOM | 901 | CG | ARG A | 138 | 43.140 | 52.934 | 40.660 | 0.00 | 41.86 | A |
| ATOM | 902 | CD | ARG A | 138 | 43.070 | 53.989 | 39.550 | 0.00 | 42.76 | A |
| ATOM | 903 | NE | ARG A | 138 | 44.301 | 54.072 | 38.764 | 0.00 | 43.72 | A |
| ATOM | 904 | CZ | ARG A | 138 | 44.443 | 54.802 | 37.659 | 0.00 | 44.10 | A |
| ATOM | 905 | NH1 | ARG A | 138 | 43.430 | 55.523 | 37.196 | 0.00 | 44.35 | A |
| ATOM | 906 | NH2 | ARG A | 138 | 45.602 | 54.812 | 37.015 | 0.00 | 44.35 | A |
| ATOM | 907 | C | ARG A | 138 | 45.706 | 54.791 | 42.895 | 1.00 | 39.80 | A |
| ATOM | 908 | O | ARG A | 138 | 45.705 | 56.016 | 42.762 | 1.00 | 39.09 | A |
| ATOM | 909 | N | THR A | 139 | 46.083 | 54.177 | 44.008 | 1.00 | 39.87 | A |
| ATOM | 910 | CA | THR A | 139 | 46.544 | 54.877 | 45.193 | 1.00 | 39.93 | A |
| ATOM | 911 | CB | THR A | 139 | 47.058 | 53.872 | 46.234 | 1.00 | 40.30 | A |
| ATOM | 912 | OG1 | THR A | 139 | 45.971 | 53.054 | 46.689 | 1.00 | 40.58 | A |
| ATOM | 913 | CG2 | THR A | 139 | 47.684 | 54.602 | 47.420 | 1.00 | 41.79 | A |
| ATOM | 914 | C | THR A | 139 | 47.674 | 55.849 | 44.881 | 1.00 | 41.13 | A |
| ATOM | 915 | O | THR A | 139 | 47.752 | 56.941 | 45.455 | 1.00 | 42.14 | A |
| ATOM | 916 | N | PHE A | 140 | 48.559 | 55.443 | 43.978 | 1.00 | 41.08 | A |
| ATOM | 917 | CA | PHE A | 140 | 49.705 | 56.267 | 43.607 | 1.00 | 39.57 | A |
| ATOM | 918 | CB | PHE A | 140 | 50.986 | 55.449 | 43.755 | 1.00 | 38.91 | A |
| ATOM | 919 | CG | PHE A | 140 | 51.220 | 54.943 | 45.145 | 1.00 | 38.38 | A |
| ATOM | 920 | CD1 | PHE A | 140 | 51.519 | 55.824 | 46.174 | 1.00 | 38.13 | A |
| ATOM | 921 | CD2 | PHE A | 140 | 51.122 | 53.588 | 45.432 | 1.00 | 37.94 | A |
| ATOM | 922 | GE1 | PHE A | 140 | 51.722 | 55.364 | 47.464 | 1.00 | 38.18 | A |
| ATOM | 923 | GE2 | PHE A | 140 | 51.323 | 53.118 | 46.721 | 1.00 | 37.42 | A |
| ATOM | 924 | CZ | PHE A | 140 | 51.622 | 54.008 | 47.739 | 1.00 | 38.51 | A |
| ATOM | 925 | C | PHE A | 140 | 49.600 | 56.779 | 42.185 | 1.00 | 39.01 | A |
| ATOM | 926 | O | PHE A | 140 | 50.512 | 57.434 | 41.685 | 1.00 | 39.06 | A |
| ATOM | 927 | N | ASN A | 141 | 48.485 | 56.483 | 41.537 | 1.00 | 38.54 | A |
| ATOM | 928 | CA | ASN A | 141 | 48.294 | 56.894 | 40.162 | 1.00 | 38.58 | A |
| ATOM | 929 | CB | ASN A | 141 | 48.220 | 58.407 | 40.049 | 1.00 | 39.46 | A |
| ATOM | 930 | CG | ASN A | 141 | 47.787 | 58.847 | 38.669 | 1.00 | 41.82 | A |
| ATOM | 931 | OD1 | ASN A | 141 | 46.655 | 58.592 | 38.257 | 1.00 | 44.53 | A |
| ATOM | 932 | ND2 | ASN A | 141 | 48.687 | 59.495 | 37.938 | 1.00 | 42.34 | A |
| ATOM | 933 | C | ASN A | 141 | 49.439 | 56.378 | 39.296 | 1.00 | 38.74 | A |
| ATOM | 934 | O | ASN A | 141 | 50.031 | 57.122 | 38.503 | 1.00 | 38.59 | A |
| ATOM | 935 | N | ILE A | 142 | 49.738 | 55.093 | 39.459 | 1.00 | 38.05 | A |
| ATOM | 936 | CA | ILE A | 142 | 50.800 | 54.429 | 38.715 | 1.00 | 37.68 | A |
| ATOM | 937 | CB | ILE A | 142 | 51.425 | 53.321 | 39.561 | 1.00 | 36.00 | A |
| ATOM | 938 | CG2 | ILE A | 142 | 52.451 | 52.563 | 38.755 | 1.00 | 34.95 | A |
| ATOM | 939 | CG1 | ILE A | 142 | 52.021 | 53.921 | 40.829 | 1.00 | 35.82 | A |
| ATOM | 940 | CD1 | ILE A | 142 | 52.449 | 52.875 | 41.837 | 1.00 | 36.82 | A |
| ATOM | 941 | C | ILE A | 142 | 50.239 | 53.819 | 37.440 | 1.00 | 38.81 | A |
| ATOM | 942 | O | ILE A | 142 | 49.121 | 53.321 | 37.441 | 1.00 | 39.45 | A |
| ATOM | 943 | N | VAL A | 143 | 51.022 | 53.855 | 36.361 | 1.00 | 40.31 | A |
| ATOM | 944 | CA | VAL A | 143 | 50.620 | 53.310 | 35.053 | 1.00 | 40.27 | A |
| ATOM | 945 | CB | VAL A | 143 | 51.373 | 54.017 | 33.905 | 1.00 | 42.11 | A |
| ATOM | 946 | CG1 | VAL A | 143 | 51.035 | 53.348 | 32.567 | 1.00 | 42.72 | A |
| ATOM | 947 | CG2 | VAL A | 143 | 51.016 | 55.493 | 33.877 | 1.00 | 41.43 | A |
| ATOM | 948 | C | VAL A | 143 | 50.868 | 51.808 | 34.881 | 1.00 | 39.19 | A |
| ATOM | 949 | O | VAL A | 143 | 51.955 | 51.309 | 35.172 | 1.00 | 38.15 | A |
| ATOM | 950 | N | ASN A | 144 | 49.869 | 51.107 | 34.359 | 1.00 | 38.94 | A |
| ATOM | 951 | CA | ASN A | 144 | 49.968 | 49.670 | 34.137 | 1.00 | 39.16 | A |
| ATOM | 952 | CB | ASN A | 144 | 48.575 | 49.048 | 34.256 | 1.00 | 39.51 | A |
| ATOM | 953 | CG | ASN A | 144 | 48.570 | 47.573 | 33.959 | 1.00 | 40.78 | A |
| ATOM | 954 | OD1 | ASN A | 144 | 49.586 | 46.903 | 34.090 | 1.00 | 44.00 | A |
| ATOM | 955 | ND2 | ASN A | 144 | 47.421 | 47.052 | 33.574 | 1.00 | 41.05 | A |
| ATOM | 956 | C | ASN A | 144 | 50.582 | 49.341 | 32.775 | 1.00 | 38.55 | A |
| ATOM | 957 | O | ASN A | 144 | 49.876 | 48.975 | 31.839 | 1.00 | 37.91 | A |
| ATOM | 958 | N | ASP A | 145 | 51.902 | 49.448 | 32.685 | 1.00 | 38.25 | A |
| ATOM | 959 | CA | ASP A | 145 | 52.624 | 49.190 | 31.440 | 1.00 | 39.85 | A |
| ATOM | 960 | CB | ASP A | 145 | 53.996 | 49.862 | 31.498 | 1.00 | 39.46 | A |
| ATOM | 961 | CG | ASP A | 145 | 54.766 | 49.490 | 32.750 | 1.00 | 39.69 | A |
| ATOM | 962 | OD1 | ASP A | 145 | 54.213 | 48.735 | 33.587 | 1.00 | 39.21 | A |
| ATOM | 963 | OD2 | ASP A | 145 | 55.918 | 49.947 | 32.895 | 1.00 | 38.21 | A |
| ATOM | 964 | C | ASP A | 145 | 52.798 | 47.712 | 31.092 | 1.00 | 40.13 | A |
| ATOM | 965 | O | ASP A | 145 | 53.702 | 47.343 | 30.337 | 1.00 | 39.56 | A |
| ATOM | 966 | N | PHE A | 146 | 51.938 | 46.870 | 31.652 | 1.00 | 40.47 | A |
| ATOM | 967 | CA | PHE A | 146 | 51.978 | 45.440 | 31.377 | 1.00 | 40.10 | A |
| ATOM | 968 | CB | PHE A | 146 | 51.371 | 44.644 | 32.541 | 1.00 | 39.31 | A |
| ATOM | 969 | CG | PHE A | 146 | 52.301 | 44.453 | 33.699 | 1.00 | 40.02 | A |
| ATOM | 970 | CD1 | PHE A | 146 | 53.534 | 43.850 | 33.520 | 1.00 | 39.21 | A |
| ATOM | 971 | CD2 | PHE A | 146 | 51.952 | 44.889 | 34.971 | 1.00 | 39.97 | A |
| ATOM | 972 | CE1 | PHE A | 146 | 54.407 | 43.690 | 34.591 | 1.00 | 39.41 | A |
| ATOM | 973 | CE2 | PHE A | 146 | 52.827 | 44.731 | 36.051 | 1.00 | 38.93 | A |
| ATOM | 974 | CZ | PHE A | 146 | 54.053 | 44.131 | 35.858 | 1.00 | 38.17 | A |
| ATOM | 975 | C | PHE A | 146 | 51.167 | 45.171 | 30.113 | 1.00 | 40.82 | A |
| ATOM | 976 | O | PHE A | 146 | 50.013 | 45.603 | 29.999 | 1.00 | 41.89 | A |
| ATOM | 977 | N | THR A | 147 | 51.773 | 44.476 | 29.154 | 1.00 | 40.38 | A |
| ATOM | 978 | CA | THR A | 147 | 51.073 | 44.137 | 27.922 | 1.00 | 38.81 | A |
| ATOM | 979 | CB | THR A | 147 | 51.999 | 43.509 | 26.877 | 1.00 | 38.98 | A |
| ATOM | 980 | OG1 | THR A | 147 | 52.651 | 42.373 | 27.456 | 1.00 | 39.24 | A |
| ATOM | 981 | CG2 | THR A | 147 | 53.030 | 44.498 | 26.395 | 1.00 | 36.50 | A |
| ATOM | 982 | C | THR A | 147 | 50.062 | 43.070 | 28.286 | 1.00 | 38.78 | A |
| ATOM | 983 | O | THR A | 147 | 50.233 | 42.343 | 29.268 | 1.00 | 37.08 | A |
| ATOM | 984 | N | PRO A | 148 | 49.003 | 42.943 | 27.485 | 1.00 | 39.53 | A |
| ATOM | 985 | CD | PRO A | 148 | 48.718 | 43.722 | 26.266 | 1.00 | 39.08 | A |
| ATOM | 986 | CA | PRO A | 148 | 47.958 | 41.947 | 27.732 | 1.00 | 40.59 | A |
| ATOM | 987 | CB | PRO A | 148 | 47.166 | 41.970 | 26.428 | 1.00 | 39.52 | A |
| ATOM | 988 | CG | PRO A | 148 | 47.260 | 43.419 | 26.034 | 1.00 | 39.40 | A |
| ATOM | 989 | C | PRO A | 148 | 48.505 | 40.545 | 28.078 | 1.00 | 42.69 | A |
| ATOM | 990 | O | PRO A | 148 | 48.007 | 39.876 | 28.984 | 1.00 | 43.05 | A |
| ATOM | 991 | N | GLU A | 149 | 49.546 | 40.123 | 27.370 | 1.00 | 45.32 | A |
| ATOM | 992 | CA | GLU A | 149 | 50.155 | 38.814 | 27.572 | 1.00 | 46.47 | A |
| ATOM | 993 | CB | GLU A | 149 | 51.059 | 38.496 | 26.395 | 1.00 | 44.18 | A |
| ATOM | 994 | CG | GLU A | 149 | 51.681 | 37.137 | 26.430 | 1.00 | 43.56 | A |
| ATOM | 995 | CD | GLU A | 149 | 52.660 | 36.955 | 25.311 | 1.00 | 43.86 | A |
| ATOM | 996 | OE1 | GLU A | 149 | 53.660 | 37.699 | 25.279 | 1.00 | 43.12 | A |
| ATOM | 997 | OE2 | GLU A | 149 | 52.430 | 36.075 | 24.458 | 1.00 | 44.44 | A |
| ATOM | 998 | C | GLU A | 149 | 50.974 | 38.773 | 28.845 | 1.00 | 48.79 | A |
| ATOM | 999 | O | GLU A | 149 | 50.852 | 37.856 | 29.660 | 1.00 | 49.11 | A |
| ATOM | 1000 | N | GLU A | 150 | 51.826 | 39.773 | 29.006 | 1.00 | 52.88 | A |
| ATOM | 1001 | CA | GLU A | 150 | 52.676 | 39.855 | 30.180 | 1.00 | 56.79 | A |
| ATOM | 1002 | CB | GLU A | 150 | 53.548 | 41.105 | 30.090 | 1.00 | 56.56 | A |
| ATOM | 1003 | CG | GLU A | 150 | 54.707 | 41.109 | 31.045 | 1.00 | 57.96 | A |
| ATOM | 1004 | CD | GLU A | 150 | 55.774 | 40.103 | 30.660 | 1.00 | 59.33 | A |
| ATOM | 1005 | OE1 | GLU A | 150 | 55.489 | 38.887 | 30.639 | 1.00 | 60.34 | A |
| ATOM | 1006 | OE2 | GLU A | 150 | 56.907 | 40.533 | 30.372 | 1.00 | 60.87 | A |
| ATOM | 1007 | C | GLU A | 150 | 51.804 | 39.913 | 31.433 | 1.00 | 59.23 | A |
| ATOM | 1008 | O | GLU A | 150 | 52.187 | 39.417 | 32.488 | 1.00 | 59.36 | A |
| ATOM | 1009 | N | GLU A | 151 | 50.627 | 40.519 | 31.305 | 1.00 | 62.33 | A |
| ATOM | 1010 | CA | GLU A | 151 | 49.701 | 40.651 | 32.424 | 1.00 | 64.96 | A |
| ATOM | 1011 | CB | GLU A | 151 | 48.477 | 41.452 | 31.982 | 1.00 | 65.44 | A |
| ATOM | 1012 | CG | GLU A | 151 | 48.102 | 42.579 | 32.923 | 1.00 | 66.38 | A |
| ATOM | 1013 | CD | GLU A | 151 | 47.141 | 43.563 | 32.287 | 1.00 | 67.52 | A |
| ATOM | 1014 | OE1 | GLU A | 151 | 47.510 | 44.187 | 31.269 | 1.00 | 67.49 | A |
| ATOM | 1015 | OE2 | GLU A | 151 | 46.014 | 43.713 | 32.803 | 1.00 | 68.18 | A |
| ATOM | 1016 | C | GLU A | 151 | 49.270 | 39.286 | 32.954 | 1.00 | 66.62 | A |
| ATOM | 1017 | O | GLU A | 151 | 49.543 | 38.943 | 34.104 | 1.00 | 66.87 | A |
| ATOM | 1018 | N | ALA A | 152 | 48.602 | 38.512 | 32.106 | 1.00 | 68.29 | A |
| ATOM | 1019 | CA | ALA A | 152 | 48.137 | 37.181 | 32.474 | 1.00 | 70.15 | A |
| ATOM | 1020 | CB | ALA A | 152 | 47.511 | 36.503 | 31.268 | 1.00 | 70.09 | A |
| ATOM | 1021 | C | ALA A | 152 | 49.280 | 36.327 | 33.014 | 1.00 | 71.79 | A |
| ATOM | 1022 | O | ALA A | 152 | 49.085 | 35.503 | 33.903 | 1.00 | 71.18 | A |
| ATOM | 1023 | N | ALA A | 153 | 50.477 | 36.526 | 32.475 | 1.00 | 74.38 | A |
| ATOM | 1024 | CA | ALA A | 153 | 51.632 | 35.761 | 32.917 | 1.00 | 76.96 | A |
| ATOM | 1025 | CB | ALA A | 153 | 52.834 | 36.056 | 32.020 | 1.00 | 76.52 | A |
| ATOM | 1026 | C | ALA A | 153 | 51.969 | 36.069 | 34.376 | 1.00 | 79.23 | A |
| ATOM | 1027 | O | ALA A | 153 | 53.040 | 35.700 | 34.863 | 1.00 | 80.18 | A |
| ATOM | 1028 | N | ILE A | 154 | 51.061 | 36.759 | 35.067 | 1.00 | 80.86 | A |
| ATOM | 1029 | CA | ILE A | 154 | 51.257 | 37.103 | 36.479 | 1.00 | 82.60 | A |
| ATOM | 1030 | CB | ILE A | 154 | 51.680 | 38.592 | 36.656 | 1.00 | 81.99 | A |
| ATOM | 1031 | CG2 | ILE A | 154 | 51.889 | 38.898 | 38.134 | 0.00 | 82.34 | A |
| ATOM | 1032 | CG1 | ILE A | 154 | 52.986 | 38.866 | 35.905 | 1.00 | 81.41 | A |
| ATOM | 1033 | CD1 | ILE A | 154 | 53.347 | 40.336 | 35.822 | 0.00 | 81.80 | A |
| ATOM | 1034 | C | ILE A | 154 | 49.976 | 36.834 | 37.285 | 1.00 | 83.80 | A |
| ATOM | 1035 | O | ILE A | 154 | 49.036 | 37.633 | 37.272 | 1.00 | 83.56 | A |
| ATOM | 1036 | N | ARG A | 155 | 49.956 | 35.697 | 37.982 | 1.00 | 85.60 | A |
| ATOM | 1037 | CA | ARG A | 155 | 48.807 | 35.284 | 38.783 | 1.00 | 86.76 | A |
| ATOM | 1038 | CB | ARG A | 155 | 48.632 | 36.208 | 39.992 | 0.00 | 87.40 | A |
| ATOM | 1039 | CG | ARG A | 155 | 49.431 | 35.788 | 41.222 | 0.00 | 88.39 | A |
| ATOM | 1040 | CD | ARG A | 155 | 50.925 | 35.725 | 40.946 | 0.00 | 89.22 | A |
| ATOM | 1041 | NE | ARG A | 155 | 51.668 | 35.196 | 42.087 | 0.00 | 89.99 | A |
| ATOM | 1042 | CZ | ARG A | 155 | 51.561 | 33.949 | 42.535 | 0.00 | 90.38 | A |
| ATOM | 1043 | NH1 | ARG A | 155 | 50.740 | 33.095 | 41.937 | 0.00 | 90.63 | A |
| ATOM | 1044 | NH2 | ARG A | 155 | 52.272 | 33.555 | 43.584 | 0.00 | 90.63 | A |
| ATOM | 1045 | C | ARG A | 155 | 47.543 | 35.286 | 37.933 | 1.00 | 87.04 | A |
| ATOM | 1046 | O | ARG A | 155 | 46.710 | 36.199 | 38.125 | 1.00 | 87.33 | A |
| ATOM | 1047 | OXT | ARG A | 155 | 47.413 | 34.380 | 37.075 | 1.00 | 86.76 | A |
| ATOM | 1048 | CB | LED B | 270 | 49.350 | 65.486 | 42.241 | 1.00 | 56.04 | B |
| ATOM | 1049 | CG | LED B | 270 | 48.859 | 64.640 | 41.059 | 1.00 | 56.55 | B |
| ATOM | 1050 | CD1 | LED B | 270 | 47.337 | 64.629 | 41.020 | 1.00 | 55.59 | B |
| ATOM | 1051 | CD2 | LEU B | 270 | 49.424 | 65.209 | 39.762 | 1.00 | 56.75 | B |
| ATOM | 1052 | C | LED B | 270 | 49.537 | 63.882 | 44.208 | 1.00 | 54.79 | B |
| ATOM | 1053 | O | LEU B | 270 | 50.136 | 63.918 | 45.287 | 1.00 | 55.23 | B |
| ATOM | 1054 | N | LED B | 270 | 49.056 | 66.304 | 44.560 | 1.00 | 56.05 | B |
| ATOM | 1055 | CA | LED B | 270 | 48.835 | 65.142 | 43.652 | 1.00 | 55.53 | B |
| ATOM | 1056 | N | LYS B | 271 | 49.463 | 62.779 | 43.462 | 1.00 | 52.96 | B |
| ATOM | 1057 | CA | LYS B | 271 | 50.053 | 61.495 | 43.865 | 1.00 | 49.38 | B |
| ATOM | 1058 | CB | LYS B | 271 | 49.292 | 60.338 | 43.210 | 1.00 | 48.76 | B |
| ATOM | 1059 | CG | LYS B | 271 | 47.793 | 60.523 | 43.065 | 1.00 | 48.20 | B |
| ATOM | 1060 | CD | LYS B | 271 | 47.049 | 59.967 | 44.264 | 1.00 | 48.26 | B |
| ATOM | 1061 | CE | LYS B | 271 | 45.545 | 59.892 | 44.014 | 1.00 | 46.84 | B |
| ATOM | 1062 | NZ | LYS B | 271 | 45.188 | 59.152 | 42.766 | 1.00 | 47.61 | B |
| ATOM | 1063 | C | LYS B | 271 | 51.521 | 61.364 | 43.462 | 1.00 | 47.55 | B |
| ATOM | 1064 | O | LYS B | 271 | 52.069 | 62.218 | 42.764 | 1.00 | 47.27 | B |
| ATOM | 1065 | N | ARG B | 272 | 52.138 | 60.263 | 43.883 | 1.00 | 45.44 | B |
| ATOM | 1066 | CA | ARG B | 272 | 53.529 | 59.981 | 43.558 | 1.00 | 42.97 | B |
| ATOM | 1067 | CB | ARG B | 272 | 54.444 | 60.845 | 44.404 | 1.00 | 42.52 | B |
| ATOM | 1068 | CG | ARG B | 272 | 55.897 | 60.542 | 44.181 | 1.00 | 44.05 | B |
| ATOM | 1069 | CD | ARG B | 272 | 56.741 | 61.677 | 44.691 | 1.00 | 45.19 | B |
| ATOM | 1070 | NE | ARG B | 272 | 57.927 | 61.836 | 43.866 | 1.00 | 46.68 | B |
| ATOM | 1071 | CZ | ARG B | 272 | 58.416 | 63.009 | 43.491 | 1.00 | 47.36 | B |
| ATOM | 1072 | NH1 | ARG B | 272 | 57.816 | 64.130 | 43.868 | 1.00 | 46.52 | B |
| ATOM | 1073 | NH2 | ARG B | 272 | 59.505 | 63.056 | 42.737 | 1.00 | 48.34 | B |
| ATOM | 1074 | C | ARG B | 272 | 53.911 | 58.517 | 43.753 | 1.00 | 41.43 | B |
| ATOM | 1075 | O | ARG B | 272 | 53.678 | 57.944 | 44.816 | 1.00 | 41.22 | B |
| ATOM | 1076 | N | ASP B | 273 | 54.495 | 57.911 | 42.724 | 1.00 | 39.99 | B |
| ATOM | 1077 | CA | ASP B | 273 | 54.914 | 56.517 | 42.821 | 1.00 | 37.49 | B |
| ATOM | 1078 | CB | ASP B | 273 | 55.435 | 56.006 | 41.487 | 1.00 | 38.41 | B |
| ATOM | 1079 | CG | ASP B | 273 | 55.631 | 54.508 | 41.482 | 1.00 | 40.70 | B |
| ATOM | 1080 | OD1 | ASP B | 273 | 56.269 | 53.969 | 42.423 | 1.00 | 39.39 | B |
| ATOM | 1081 | OD2 | ASP B | 273 | 55.143 | 53.874 | 40.525 | 1.00 | 43.85 | B |
| ATOM | 1082 | C | ASP B | 273 | 56.040 | 56.474 | 43.829 | 1.00 | 35.40 | B |
| ATOM | 1083 | O | ASP B | 273 | 57.218 | 56.453 | 43.463 | 1.00 | 33.99 | B |
| ATOM | 1084 | N | LEU B | 274 | 55.671 | 56.460 | 45.101 | 1.00 | 33.28 | B |
| ATOM | 1085 | CA | LEU B | 274 | 56.646 | 56.450 | 46.170 | 1.00 | 32.55 | B |
| ATOM | 1086 | CB | LEU B | 274 | 55.937 | 56.322 | 47.514 | 1.00 | 32.85 | B |
| ATOM | 1087 | CG | LEU B | 274 | 55.728 | 57.659 | 48.229 | 1.00 | 34.16 | B |
| ATOM | 1088 | CD1 | LEU B | 274 | 54.930 | 58.598 | 47.361 | 1.00 | 33.65 | B |
| ATOM | 1089 | CD2 | LEU B | 274 | 55.035 | 57.421 | 49.561 | 1.00 | 35.07 | B |
| ATOM | 1090 | C | LEU B | 274 | 57.723 | 55.391 | 46.066 | 1.00 | 31.70 | B |
| ATOM | 1091 | O | LEU B | 274 | 58.903 | 55.687 | 46.250 | 1.00 | 31.68 | B |
| ATOM | 1092 | N | ILE B | 275 | 57.338 | 54.160 | 45.756 | 1.00 | 31.28 | B |
| ATOM | 1093 | CA | ILE B | 275 | 58.338 | 53.115 | 45.687 | 1.00 | 30.61 | B |
| ATOM | 1094 | CB | ILE B | 275 | 57.703 | 51.700 | 45.636 | 1.00 | 31.77 | B |
| ATOM | 1095 | CG2 | ILE B | 275 | 56.364 | 51.696 | 46.322 | 1.00 | 29.79 | B |
| ATOM | 1096 | CG1 | ILE B | 275 | 57.579 | 51.245 | 44.196 | 1.00 | 34.36 | B |
| ATOM | 1097 | CD1 | ILE B | 275 | 58.058 | 49.838 | 44.000 | 1.00 | 34.82 | B |
| ATOM | 1098 | C | ILE B | 275 | 59.325 | 53.301 | 44.535 | 1.00 | 29.10 | B |
| ATOM | 1099 | O | ILE B | 275 | 60.495 | 52.944 | 44.675 | 1.00 | 28.41 | B |
| ATOM | 1100 | N | THR B | 276 | 58.893 | 53.846 | 43.398 | 1.00 | 28.07 | B |
| ATOM | 1101 | CA | THR B | 276 | 59.875 | 54.056 | 42.336 | 1.00 | 29.79 | B |
| ATOM | 1102 | CB | THR B | 276 | 59.259 | 54.160 | 40.906 | 1.00 | 29.82 | B |
| ATOM | 1103 | OG1 | THR B | 276 | 58.327 | 55.236 | 40.859 | 1.00 | 33.40 | B |
| ATOM | 1104 | CG2 | THR B | 276 | 58.569 | 52.869 | 40.507 | 1.00 | 31.42 | B |
| ATOM | 1105 | C | THR B | 276 | 60.718 | 55.314 | 42.580 | 1.00 | 28.87 | B |
| ATOM | 1106 | O | THR B | 276 | 61.885 | 55.341 | 42.222 | 1.00 | 29.74 | B |
| ATOM | 1107 | N | SER B | 277 | 60.145 | 56.333 | 43.211 | 1.00 | 27.47 | B |
| ATOM | 1108 | CA | SER B | 277 | 60.867 | 57.567 | 43.454 | 1.00 | 27.08 | B |
| ATOM | 1109 | CB | SER B | 277 | 59.877 | 58.706 | 43.664 | 1.00 | 28.54 | B |
| ATOM | 1110 | OG | SER B | 277 | 59.027 | 58.861 | 42.533 | 1.00 | 29.07 | B |
| ATOM | 1111 | C | SER B | 277 | 61.843 | 57.511 | 44.622 | 1.00 | 28.04 | B |
| ATOM | 1112 | O | SER B | 277 | 62.850 | 58.200 | 44.607 | 1.00 | 29.44 | B |
| ATOM | 1113 | N | LEU B | 278 | 61.546 | 56.717 | 45.644 | 1.00 | 28.55 | B |
| ATOM | 1114 | CA | LEU B | 278 | 62.443 | 56.606 | 46.786 | 1.00 | 28.04 | B |
| ATOM | 1115 | CB | LEU B | 278 | 61.777 | 55.854 | 47.921 | 1.00 | 27.87 | B |
| ATOM | 1116 | CG | LEU B | 278 | 61.098 | 56.650 | 49.043 | 1.00 | 29.93 | B |
| ATOM | 1117 | CD1 | LEU B | 278 | 62.130 | 57.593 | 49.658 | 1.00 | 30.19 | B |
| ATOM | 1118 | CD2 | LEU B | 278 | 59.898 | 57.407 | 48.526 | 1.00 | 28.70 | B |
| ATOM | 1119 | C | LEU B | 278 | 63.697 | 55.857 | 46.401 | 1.00 | 29.78 | B |
| ATOM | 1120 | O | LEU B | 278 | 63.728 | 55.140 | 45.398 | 1.00 | 30.41 | B |
| ATOM | 1121 | N | PRO B | 279 | 64.771 | 56.039 | 47.171 | 1.00 | 30.02 | B |
| ATOM | 1122 | CD | PRO B | 279 | 65.103 | 57.050 | 48.187 | 1.00 | 29.94 | B |
| ATOM | 1123 | CA | PRO B | 279 | 65.948 | 55.281 | 46.764 | 1.00 | 30.32 | B |
| ATOM | 1124 | CB | PRO B | 279 | 67.068 | 55.907 | 47.584 | 1.00 | 29.20 | B |
| ATOM | 1125 | CG | PRO B | 279 | 66.371 | 56.499 | 48.763 | 1.00 | 31.41 | B |
| ATOM | 1126 | C | PRO B | 279 | 65.690 | 53.813 | 47.094 | 1.00 | 32.64 | B |
| ATOM | 1127 | O | PRO B | 279 | 64.946 | 53.494 | 48.026 | 1.00 | 32.21 | B |
| ATOM | 1128 | N | PHE B | 280 | 66.291 | 52.927 | 46.308 | 1.00 | 34.93 | B |
| ATOM | 1129 | CA | PHE B | 280 | 66.122 | 51.495 | 46.476 | 1.00 | 35.83 | B |
| ATOM | 1130 | CB | PHE B | 280 | 67.177 | 50.745 | 45.671 | 1.00 | 35.18 | B |
| ATOM | 1131 | CG | PHE B | 280 | 67.040 | 49.266 | 45.752 | 1.00 | 35.73 | B |
| ATOM | 1132 | CD1 | PHE B | 280 | 65.871 | 48.644 | 45.307 | 1.00 | 35.82 | B |
| ATOM | 1133 | CD2 | PHE B | 280 | 68.050 | 48.493 | 46.319 | 1.00 | 34.88 | B |
| ATOM | 1134 | CE1 | PHE B | 280 | 65.703 | 47.266 | 45.430 | 1.00 | 37.94 | B |
| ATOM | 1135 | CE2 | PHE B | 280 | 67.897 | 47.111 | 46.452 | 1.00 | 36.63 | B |
| ATOM | 1136 | CZ | PHE B | 280 | 66.717 | 46.489 | 46.006 | 1.00 | 36.74 | B |
| ATOM | 1137 | C | PHE B | 280 | 66.172 | 51.017 | 47.917 | 1.00 | 37.41 | B |
| ATOM | 1138 | O | PHE B | 280 | 65.263 | 50.331 | 48.371 | 1.00 | 39.64 | B |
| ATOM | 1139 | N | GLU B | 281 | 67.235 | 51.366 | 48.634 | 1.00 | 38.02 | B |
| ATOM | 1140 | CA | GLU B | 281 | 67.391 | 50.934 | 50.017 | 1.00 | 38.96 | B |
| ATOM | 1141 | CB | GLU B | 281 | 68.622 | 51.584 | 50.659 | 1.00 | 42.67 | B |
| ATOM | 1142 | CG | GLU B | 281 | 69.932 | 51.424 | 49.875 | 1.00 | 49.17 | B |
| ATOM | 1143 | CD | GLU B | 281 | 70.188 | 49.992 | 49.394 | 1.00 | 51.93 | B |
| ATOM | 1144 | OE1 | GLU B | 281 | 69.840 | 49.030 | 50.127 | 1.00 | 51.55 | B |
| ATOM | 1145 | OE2 | GLU B | 281 | 70.747 | 49.845 | 48.276 | 1.00 | 53.97 | B |
| ATOM | 1146 | C | GLU B | 281 | 66.169 | 51.251 | 50.859 | 1.00 | 38.70 | B |
| ATOM | 1147 | O | GLU B | 281 | 65.863 | 50.516 | 51.796 | 1.00 | 40.56 | B |
| ATOM | 1148 | N | ILE B | 282 | 65.472 | 52.338 | 50.531 | 1.00 | 36.39 | B |
| ATOM | 1149 | CA | ILE B | 282 | 64.289 | 52.728 | 51.291 | 1.00 | 33.47 | B |
| ATOM | 1150 | CB | ILE B | 282 | 64.019 | 54.250 | 51.194 | 1.00 | 33.48 | B |
| ATOM | 1151 | CG2 | ILE B | 282 | 62.633 | 54.581 | 51.721 | 1.00 | 31.07 | B |
| ATOM | 1152 | CG1 | ILE B | 282 | 65.091 | 55.007 | 51.974 | 1.00 | 33.33 | B |
| ATOM | 1153 | CD1 | ILE B | 282 | 64.849 | 56.494 | 52.090 | 1.00 | 35.69 | B |
| ATOM | 1154 | C | ILE B | 282 | 63.030 | 51.984 | 50.891 | 1.00 | 32.60 | B |
| ATOM | 1155 | O | ILE B | 282 | 62.238 | 51.614 | 51.748 | 1.00 | 33.74 | B |
| ATOM | 1156 | N | SER B | 283 | 62.833 | 51.753 | 49.602 | 1.00 | 32.28 | B |
| ATOM | 1157 | CA | SER B | 283 | 61.632 | 51.052 | 49.180 | 1.00 | 31.76 | B |
| ATOM | 1158 | CB | SER B | 283 | 61.531 | 51.043 | 47.665 | 1.00 | 30.24 | B |
| ATOM | 1159 | OG | SER B | 283 | 60.948 | 52.260 | 47.227 | 1.00 | 30.65 | B |
| ATOM | 1160 | C | SER B | 283 | 61.554 | 49.642 | 49.732 | 1.00 | 32.64 | B |
| ATOM | 1161 | O | SER B | 283 | 60.460 | 49.145 | 50.033 | 1.00 | 31.74 | B |
| ATOM | 1162 | N | LEU B | 284 | 62.715 | 49.011 | 49.891 | 1.00 | 34.08 | B |
| ATOM | 1163 | CA | LEU B | 284 | 62.761 | 47.661 | 50.433 | 1.00 | 36.79 | B |
| ATOM | 1164 | CB | LEU B | 284 | 64.158 | 47.070 | 50.303 | 1.00 | 38.54 | B |
| ATOM | 1165 | CG | LEU B | 284 | 64.620 | 46.663 | 48.913 | 1.00 | 40.73 | B |
| ATOM | 1166 | CO1 | LEU B | 284 | 65.953 | 45.935 | 49.085 | 1.00 | 41.39 | B |
| ATOM | 1167 | CD2 | LEU B | 284 | 63.598 | 45.746 | 48.237 | 1.00 | 39.81 | B |
| ATOM | 1168 | C | LEU B | 284 | 62.331 | 47.606 | 51.903 | 1.00 | 37.57 | B |
| ATOM | 1169 | O | LEU B | 284 | 61.731 | 46.629 | 52.333 | 1.00 | 37.16 | B |
| ATOM | 1170 | N | LYS B | 285 | 62.654 | 48.636 | 52.679 | 1.00 | 37.42 | B |
| ATOM | 1171 | CA | LYS B | 285 | 62.258 | 48.638 | 54.071 | 1.00 | 37.32 | B |
| ATOM | 1172 | CB | LYS B | 285 | 62.706 | 49.927 | 54.757 | 1.00 | 37.90 | B |
| ATOM | 1173 | CG | LYS B | 285 | 64.221 | 50.101 | 54.711 | 1.00 | 41.29 | B |
| ATOM | 1174 | CD | LYS B | 285 | 64.714 | 51.417 | 55.305 | 1.00 | 43.06 | B |
| ATOM | 1175 | CE | LYS B | 285 | 64.504 | 51.487 | 56.811 | 1.00 | 44.78 | B |
| ATOM | 1176 | NZ | LYS B | 285 | 63.347 | 52.365 | 57.139 | 1.00 | 44.95 | B |
| ATOM | 1177 | C | LYS B | 285 | 60.754 | 48.531 | 54.078 | 1.00 | 36.58 | B |
| ATOM | 1178 | O | LYS B | 285 | 60.170 | 47.774 | 54.859 | 1.00 | 36.75 | B |
| ATOM | 1179 | N | ILE B | 286 | 60.137 | 49.268 | 53.163 | 1.00 | 34.81 | B |
| ATOM | 1180 | CA | ILE B | 286 | 58.687 | 49.286 | 53.038 | 1.00 | 34.65 | B |
| ATOM | 1181 | CB | ILE B | 286 | 58.233 | 50.340 | 51.988 | 1.00 | 35.18 | B |
| ATOM | 1182 | CG2 | ILE B | 286 | 56.723 | 50.435 | 51.961 | 1.00 | 34.75 | B |
| ATOM | 1183 | CG1 | ILE B | 286 | 58.778 | 51.721 | 52.361 | 1.00 | 36.78 | B |
| ATOM | 1184 | CD1 | ILE B | 286 | 58.517 | 52.777 | 51.304 | 1.00 | 35.35 | B |
| ATOM | 1185 | C | ILE B | 286 | 58.141 | 47.909 | 52.646 | 1.00 | 34.49 | B |
| ATOM | 1186 | O | ILE B | 286 | 57.109 | 47.469 | 53.163 | 1.00 | 34.87 | B |
| ATOM | 1187 | N | PHE B | 287 | 58.825 | 47.232 | 51.726 | 1.00 | 32.88 | B |
| ATOM | 1188 | CA | PHE B | 287 | 58.373 | 45.915 | 51.300 | 1.00 | 30.31 | B |
| ATOM | 1189 | CB | PHE B | 287 | 59.086 | 45.501 | 50.002 | 1.00 | 26.38 | B |
| ATOM | 1190 | CG | PHE B | 287 | 58.530 | 46.184 | 48.785 | 1.00 | 23.19 | B |
| ATOM | 1191 | CD1 | PHE B | 287 | 57.189 | 46.000 | 48.444 | 1.00 | 21.80 | B |
| ATOM | 1192 | CD2 | PHE B | 287 | 59.300 | 47.078 | 48.043 | 1.00 | 20.28 | B |
| ATOM | 1193 | CE1 | PHE B | 287 | 56.617 | 46.699 | 47.397 | 1.00 | 21.72 | B |
| ATOM | 1194 | CE2 | PHE B | 287 | 58.740 | 47.788 | 46.985 | 1.00 | 21.62 | B |
| ATOM | 1195 | CZ | PHE B | 287 | 57.389 | 47.599 | 46.659 | 1.00 | 23.65 | B |
| ATOM | 1196 | C | PHE B | 287 | 58.566 | 44.899 | 52.419 | 1.00 | 31.61 | B |
| ATOM | 1197 | O | PHE B | 287 | 57.864 | 43.887 | 52.487 | 1.00 | 32.92 | B |
| ATOM | 1198 | N | ASN B | 288 | 59.498 | 45.191 | 53.320 | 1.00 | 32.92 | B |
| ATOM | 1199 | CA | ASN B | 288 | 59.747 | 44.316 | 54.460 | 1.00 | 34.33 | B |
| ATOM | 1200 | CB | ASN B | 288 | 61.117 | 44.574 | 55.067 | 1.00 | 34.27 | B |
| ATOM | 1201 | CG | ASN B | 288 | 62.228 | 43.993 | 54.243 | 1.00 | 35.15 | B |
| ATOM | 1202 | OD1 | ASN B | 288 | 62.325 | 42.768 | 54.077 | 1.00 | 34.77 | B |
| ATOM | 1203 | ND2 | ASN B | 288 | 63.086 | 44.865 | 53.717 | 1.00 | 37.02 | B |
| ATOM | 1204 | C | ASN B | 288 | 58.699 | 44.515 | 55.538 | 1.00 | 34.60 | B |
| ATOM | 1205 | O | ASN B | 288 | 58.700 | 43.801 | 56.537 | 1.00 | 35.55 | B |
| ATOM | 1206 | N | TYR B | 289 | 57.833 | 45.507 | 55.347 | 1.00 | 33.60 | B |
| ATOM | 1207 | CA | TYR B | 289 | 56.757 | 45.786 | 56.286 | 1.00 | 32.28 | B |
| ATOM | 1208 | CB | TYR B | 289 | 56.597 | 47.288 | 56.536 | 1.00 | 30.88 | B |
| ATOM | 1209 | CG | TYR B | 289 | 57.639 | 47.906 | 57.444 | 1.00 | 31.04 | B |
| ATOM | 1210 | CD1 | TYR B | 289 | 57.824 | 47.445 | 58.743 | 1.00 | 29.76 | B |
| ATOM | 1211 | CE1 | TYR B | 289 | 58.783 | 48.005 | 59.577 | 1.00 | 28.80 | B |
| ATOM | 1212 | CD2 | TYR B | 289 | 58.441 | 48.954 | 57.001 | 1.00 | 30.42 | B |
| ATOM | 1213 | CE2 | TYR B | 289 | 59.404 | 49.520 | 57.828 | 1.00 | 30.11 | B |
| ATOM | 1214 | CZ | TYR B | 289 | 59.572 | 49.036 | 59.117 | 1.00 | 29.29 | B |
| ATOM | 1215 | OH | TYR B | 289 | 60.550 | 49.565 | 59.927 | 1.00 | 27.49 | B |
| ATOM | 1216 | C | TYR B | 289 | 55.454 | 45.259 | 55.712 | 1.00 | 33.20 | B |
| ATOM | 1217 | O | TYR B | 289 | 54.381 | 45.681 | 56.131 | 1.00 | 34.61 | B |
| ATOM | 1218 | N | LEU B | 290 | 55.538 | 44.348 | 54.750 | 1.00 | 32.98 | B |
| ATOM | 1219 | CA | LEU B | 290 | 54.335 | 43.794 | 54.148 | 1.00 | 33.98 | B |
| ATOM | 1220 | CB | LEU B | 290 | 54.181 | 44.325 | 52.736 | 1.00 | 33.99 | B |
| ATOM | 1221 | CG | LEU B | 290 | 53.894 | 45.813 | 52.594 | 1.00 | 34.25 | B |
| ATOM | 1222 | CD1 | LEU B | 290 | 53.690 | 46.102 | 51.123 | 1.00 | 32.35 | B |
| ATOM | 1223 | CD2 | LEU B | 290 | 52.629 | 46.192 | 53.393 | 1.00 | 35.31 | B |
| ATOM | 1224 | C | LEU B | 290 | 54.340 | 42.284 | 54.112 | 1.00 | 35.59 | B |
| ATOM | 1225 | O | LEU B | 290 | 55.366 | 41.686 | 53.832 | 1.00 | 37.73 | B |
| ATOM | 1226 | N | GLN B | 291 | 53.200 | 41.660 | 54.390 | 1.00 | 36.68 | B |
| ATOM | 1227 | CA | GLN B | 291 | 53.126 | 40.198 | 54.364 | 1.00 | 37.99 | B |
| ATOM | 1228 | CB | GLN B | 291 | 51.808 | 39.715 | 54.956 | 1.00 | 39.90 | B |
| ATOM | 1229 | CGG | LN B | 291 | 51.514 | 40.243 | 56.338 | 1.00 | 42.72 | B |
| ATOM | 1230 | CD | GLN B | 291 | 50.510 | 39.378 | 57.067 | 1.00 | 45.38 | B |
| ATOM | 1231 | OE1 | GLN B | 291 | 49.376 | 39.196 | 56.609 | 1.00 | 45.17 | B |
| ATOM | 1232 | NE2 | GLN B | 291 | 50.925 | 38.824 | 58.209 | 1.00 | 48.06 | B |
| ATOM | 1233 | C | GLN B | 291 | 53.254 | 39.696 | 52.937 | 1.00 | 37.97 | B |
| ATOM | 1234 | O | GLN B | 291 | 52.727 | 40.304 | 52.005 | 1.00 | 37.21 | B |
| ATOM | 1235 | N | PHE B | 292 | 53.924 | 38.566 | 52.769 | 1.00 | 39.53 | B |
| ATOM | 1236 | CA | PHE B | 292 | 54.154 | 38.038 | 51.430 | 1.00 | 42.10 | B |
| ATOM | 1237 | CB | PHE B | 292 | 54.592 | 36.565 | 51.510 | 1.00 | 43.75 | B |
| ATOM | 1238 | CG | PHE B | 292 | 53.463 | 35.605 | 51.727 | 1.00 | 44.90 | B |
| ATOM | 1239 | CD1 | PHE B | 292 | 52.799 | 35.038 | 50.641 | 1.00 | 43.90 | B |
| ATOM | 1240 | CD2 | PHE B | 292 | 53.050 | 35.280 | 53.014 | 1.00 | 45.10 | B |
| ATOM | 1241 | CE1 | PHE B | 292 | 51.742 | 34.161 | 50.831 | 1.00 | 43.67 | B |
| ATOM | 1242 | CE2 | PHE B | 292 | 51.992 | 34.403 | 53.217 | 1.00 | 44.65 | B |
| ATOM | 1243 | CZ | PHE B | 292 | 51.336 | 33.842 | 52.121 | 1.00 | 44.41 | B |
| ATOM | 1244 | C | PHE B | 292 | 52.990 | 38.217 | 50.441 | 1.00 | 42.01 | B |
| ATOM | 1245 | O | PHE B | 292 | 53.214 | 38.553 | 49.280 | 1.00 | 43.29 | B |
| ATOM | 1246 | N | GLU B | 293 | 51.753 | 38.018 | 50.884 | 1.00 | 41.02 | B |
| ATOM | 1247 | CA | GLU B | 293 | 50.605 | 38.176 | 49.985 | 1.00 | 40.82 | B |
| ATOM | 1248 | CB | GLU B | 293 | 49.289 | 38.028 | 50.769 | 1.00 | 41.39 | B |
| ATOM | 1249 | CG | GLU B | 293 | 49.056 | 36.617 | 51.350 | 1.00 | 42.89 | B |
| ATOM | 1250 | CD | GLU B | 293 | 49.451 | 36.480 | 52.818 | 1.00 | 43.53 | B |
| ATOM | 1251 | OE1 | GLU B | 293 | 50.507 | 37.006 | 53.222 | 1.00 | 44.09 | B |
| ATOM | 1252 | OE2 | GLU B | 293 | 48.704 | 35.825 | 53.572 | 1.00 | 45.55 | B |
| ATOM | 1253 | C | GLU B | 293 | 50.641 | 39.524 | 49.258 | 1.00 | 40.05 | B |
| ATOM | 1254 | O | GLU B | 293 | 50.375 | 39.603 | 48.056 | 1.00 | 38.85 | B |
| ATOM | 1255 | N | ASP B | 294 | 50.991 | 40.573 | 50.000 | 1.00 | 40.23 | B |
| ATOM | 1256 | CA | ASP B | 294 | 51.074 | 41.923 | 49.450 | 1.00 | 40.33 | B |
| ATOM | 1257 | CB | ASP B | 294 | 51.198 | 42.958 | 50.579 | 1.00 | 41.12 | B |
| ATOM | 1258 | CG | ASP B | 294 | 49.949 | 43.030 | 51.464 | 1.00 | 42.16 | B |
| ATOM | 1259 | OD1 | ASP B | 294 | 48.815 | 42.987 | 50.925 | 1.00 | 43.30 | B |
| ATOM | 1260 | OD2 | ASP B | 294 | 50.099 | 43.150 | 52.699 | 1.00 | 41.57 | B |
| ATOM | 1261 | C | ASP B | 294 | 52.259 | 42.074 | 48.490 | 1.00 | 38.83 | B |
| ATOM | 1262 | O | ASP B | 294 | 52.151 | 42.714 | 47.446 | 1.00 | 37.73 | B |
| ATOM | 1263 | N | ILE B | 295 | 53.390 | 41.477 | 48.840 | 1.00 | 37.53 | B |
| ATOM | 1264 | CA | ILE B | 295 | 54.561 | 41.573 | 47.994 | 1.00 | 36.43 | B |
| ATOM | 1265 | CB | ILE B | 295 | 55.751 | 40.853 | 48.638 | 1.00 | 35.75 | B |
| ATOM | 1266 | CG2 | ILE B | 295 | 56.926 | 40.801 | 47.660 | 1.00 | 35.40 | B |
| ATOM | 1267 | CG1 | ILE B | 295 | 56.107 | 41.574 | 49.950 | 1.00 | 36.24 | B |
| ATOM | 1268 | CD1 | ILE B | 295 | 57.303 | 41.033 | 50.709 | 1.00 | 36.08 | B |
| ATOM | 1269 | C | ILE B | 295 | 54.257 | 40.987 | 46.623 | 1.00 | 36.37 | B |
| ATOM | 1270 | O | ILE B | 295 | 54.707 | 41.504 | 45.593 | 1.00 | 36.01 | B |
| ATOM | 1271 | N | ILE B | 296 | 53.470 | 39.919 | 46.616 | 1.00 | 36.36 | B |
| ATOM | 1272 | CA | ILE B | 296 | 53.094 | 39.262 | 45.375 | 1.00 | 36.96 | B |
| ATOM | 1273 | CB | ILE B | 296 | 52.329 | 37.967 | 45.665 | 1.00 | 37.13 | B |
| ATOM | 1274 | CG2 | ILE B | 296 | 51.771 | 37.366 | 44.389 | 1.00 | 37.11 | B |
| ATOM | 1275 | CG1 | ILE B | 296 | 53.276 | 36.961 | 46.288 | 1.00 | 36.41 | B |
| ATOM | 1276 | CD1 | ILE B | 296 | 52.601 | 35.679 | 46.620 | 1.00 | 38.88 | B |
| ATOM | 1277 | C | ILE B | 296 | 52.256 | 40.159 | 44.473 | 1.00 | 36.89 | B |
| ATOM | 1278 | O | ILE B | 296 | 52.554 | 40.297 | 43.293 | 1.00 | 37.07 | B |
| ATOM | 1279 | N | ASN B | 297 | 51.208 | 40.769 | 45.008 | 1.00 | 37.95 | B |
| ATOM | 1280 | CA | ASN B | 297 | 50.385 | 41.649 | 44.186 | 1.00 | 38.86 | B |
| ATOM | 1281 | CB | ASN B | 297 | 49.240 | 42.246 | 44.980 | 1.00 | 41.69 | B |
| ATOM | 1282 | CG | ASN B | 297 | 48.368 | 41.213 | 45.583 | 1.00 | 43.05 | B |
| ATOM | 1283 | OD1 | ASN B | 297 | 47.914 | 40.293 | 44.897 | 1.00 | 44.19 | B |
| ATOM | 1284 | ND2 | ASN B | 297 | 48.114 | 41.345 | 46.880 | 1.00 | 43.88 | B |
| ATOM | 1285 | C | ASN B | 297 | 51.207 | 42.810 | 43.705 | 1.00 | 37.72 | B |
| ATOM | 1286 | O | ASN B | 297 | 50.953 | 43.360 | 42.632 | 1.00 | 36.87 | B |
| ATOM | 1287 | N | SER B | 298 | 52.167 | 43.207 | 44.531 | 1.00 | 36.60 | B |
| ATOM | 1288 | CA | SER B | 298 | 53.006 | 44.339 | 44.194 | 1.00 | 35.31 | B |
| ATOM | 1289 | CB | SER B | 298 | 53.926 | 44.673 | 45.366 | 1.00 | 32.10 | B |
| ATOM | 1290 | OG | SER B | 298 | 53.156 | 45.195 | 46.435 | 1.00 | 29.27 | B |
| ATOM | 1291 | C | SER B | 298 | 53.784 | 44.022 | 42.939 | 1.00 | 35.81 | B |
| ATOM | 1292 | O | SER B | 298 | 54.037 | 44.901 | 42.111 | 1.00 | 35.32 | B |
| ATOM | 1293 | N | LEU B | 299 | 54.136 | 42.750 | 42.793 | 1.00 | 36.25 | B |
| ATOM | 1294 | CA | LEU B | 299 | 54.871 | 42.291 | 41.625 | 1.00 | 36.42 | B |
| ATOM | 1295 | CB | LEU B | 299 | 55.218 | 40.814 | 41.771 | 1.00 | 35.83 | B |
| ATOM | 1296 | CG | LEU B | 299 | 56.431 | 40.528 | 42.654 | 1.00 | 36.52 | B |
| ATOM | 1297 | CD1 | LEU B | 299 | 56.674 | 39.028 | 42.727 | 1.00 | 37.64 | B |
| ATOM | 1298 | CD2 | LEU B | 299 | 57.655 | 41.224 | 42.065 | 1.00 | 35.78 | B |
| ATOM | 1299 | C | LEU B | 299 | 54.082 | 42.509 | 40.343 | 1.00 | 36.00 | B |
| ATOM | 1300 | O | LEU B | 299 | 54.660 | 42.539 | 39.256 | 1.00 | 36.92 | B |
| ATOM | 1301 | N | CLY B | 300 | 52.769 | 42.678 | 40.474 | 1.00 | 35.04 | B |
| ATOM | 1302 | CA | GLY B | 300 | 51.940 | 42.890 | 39.303 | 1.00 | 35.67 | B |
| ATOM | 1303 | C | GLY B | 300 | 51.417 | 44.305 | 39.186 | 1.00 | 35.69 | B |
| ATOM | 1304 | O | GLY B | 300 | 50.538 | 44.602 | 38.379 | 1.00 | 35.37 | B |
| ATOM | 1305 | N | VAL B | 301 | 51.958 | 45.194 | 39.998 | 1.00 | 36.32 | B |
| ATOM | 1306 | CA | VAL B | 301 | 51.523 | 46.569 | 39.969 | 1.00 | 36.62 | B |
| ATOM | 1307 | CB | VAL B | 301 | 51.953 | 47.291 | 41.247 | 1.00 | 37.56 | B |
| ATOM | 1308 | CG1 | VAL B | 301 | 51.752 | 48.789 | 41.087 | 1.00 | 37.50 | B |
| ATOM | 1309 | CG2 | VAL B | 301 | 51.133 | 46.752 | 42.436 | 1.00 | 35.20 | B |
| ATOM | 1310 | C | VAL B | 301 | 52.071 | 47.288 | 38.751 | 1.00 | 36.72 | B |
| ATOM | 1311 | O | VAL B | 301 | 51.321 | 47.910 | 38.017 | 1.00 | 37.30 | B |
| ATOM | 1312 | N | SER B | 302 | 53.378 | 47.201 | 38.534 | 1.00 | 37.60 | B |
| ATOM | 1313 | CA | SER B | 302 | 54.005 | 47.844 | 37.382 | 1.00 | 37.90 | B |
| ATOM | 1314 | CB | SER B | 302 | 54.317 | 49.311 | 37.684 | 1.00 | 37.39 | B |
| ATOM | 1315 | OG | SER B | 302 | 55.530 | 49.417 | 38.419 | 1.00 | 36.20 | B |
| ATOM | 1316 | C | SER B | 302 | 55.316 | 47.129 | 37.067 | 1.00 | 39.64 | B |
| ATOM | 1317 | O | SER B | 302 | 55.867 | 46.392 | 37.901 | 1.00 | 38.64 | B |
| ATOM | 1318 | N | GLN B | 303 | 55.821 | 47.371 | 35.861 | 1.00 | 40.39 | B |
| ATOM | 1319 | CA | GLN B | 303 | 57.087 | 46.781 | 35.428 | 1.00 | 41.46 | B |
| ATOM | 1320 | CB | GLN B | 303 | 57.436 | 47.239 | 34.013 | 1.00 | 41.20 | B |
| ATOM | 1321 | CG | GLN B | 303 | 56.696 | 46.481 | 32.950 | 1.00 | 43.64 | B |
| ATOM | 1322 | CD | GLN B | 303 | 57.199 | 45.068 | 32.815 | 1.00 | 44.54 | B |
| ATOM | 1323 | OE1 | GLN B | 303 | 57.771 | 44.517 | 33.751 | 1.00 | 46.50 | B |
| ATOM | 1324 | NE2 | GLN B | 303 | 56.986 | 44.466 | 31.650 | 1.00 | 44.23 | B |
| ATOM | 1325 | C | GLN B | 303 | 58.241 | 47.140 | 36.360 | 1.00 | 41.74 | B |
| ATOM | 1326 | O | GLN B | 303 | 59.163 | 46.345 | 36.561 | 1.00 | 41.80 | B |
| ATOM | 1327 | N | ASN B | 304 | 58.194 | 48.338 | 36.928 | 1.00 | 42.15 | B |
| ATOM | 1328 | CA | ASN B | 304 | 59.251 | 48.758 | 37.826 | 1.00 | 41.69 | B |
| ATOM | 1329 | CB | ASN B | 304 | 59.237 | 50.263 | 38.014 | 1.00 | 43.58 | B |
| ATOM | 1330 | CG | ASN B | 304 | 60.623 | 50.861 | 37.924 | 1.00 | 45.74 | B |
| ATOM | 1331 | OD1 | ASN B | 304 | 61.599 | 50.302 | 38.459 | 1.00 | 46.54 | B |
| ATOM | 1332 | ND2 | ASN B | 304 | 60.728 | 52.009 | 37.240 | 1.00 | 47.34 | B |
| ATOM | 1333 | G | ASN B | 304 | 59.130 | 48.100 | 39.174 | 1.00 | 41.16 | B |
| ATOM | 1334 | O | ASN B | 304 | 60.126 | 47.650 | 39.739 | 1.00 | 40.69 | B |
| ATOM | 1335 | N | TRP B | 305 | 57.910 | 48.060 | 39.700 | 1.00 | 40.22 | B |
| ATOM | 1336 | CA | TRP B | 305 | 57.687 | 47.439 | 40.992 | 1.00 | 39.44 | B |
| ATOM | 1337 | CB | TRP B | 305 | 56.201 | 47.464 | 41.357 | 1.00 | 40.05 | B |
| ATOM | 1338 | CG | TRP B | 305 | 55.742 | 48.762 | 41.945 | 1.00 | 41.46 | B |
| ATOM | 1339 | CD2 | TRP B | 305 | 54.933 | 48.939 | 43.120 | 1.00 | 42.85 | B |
| ATOM | 1340 | CE2 | TRP B | 305 | 54.724 | 50.324 | 43.277 | 1.00 | 43.18 | B |
| ATOM | 1341 | CE3 | TRP B | 305 | 54.363 | 48.060 | 44.054 | 1.00 | 43.70 | B |
| ATOM | 1342 | CD1 | TRP B | 305 | 55.983 | 50.007 | 41.456 | 1.00 | 41.95 | B |
| ATOM | 1343 | NE1 | TRP B | 305 | 55.375 | 50.953 | 42.248 | 1.00 | 43.45 | B |
| ATOM | 1344 | CZ2 | TRP B | 305 | 53.966 | 50.858 | 44.336 | 1.00 | 43.29 | B |
| ATOM | 1345 | CZ3 | TRP B | 305 | 53.608 | 48.591 | 45.108 | 1.00 | 43.59 | B |
| ATOM | 1346 | CH2 | TRP B | 305 | 53.419 | 49.976 | 45.237 | 1.00 | 43.13 | B |
| ATOM | 1347 | C | TRP B | 305 | 58.186 | 46.007 | 40.930 | 1.00 | 39.39 | B |
| ATOM | 1348 | O | TRP B | 305 | 58.909 | 45.556 | 41.818 | 1.00 | 40.05 | B |
| ATOM | 1349 | N | ASN B | 306 | 57.808 | 45.300 | 39.868 | 1.00 | 38.31 | B |
| ATOM | 1350 | CA | ASN B | 306 | 58.213 | 43.915 | 39.699 | 1.00 | 36.89 | B |
| ATOM | 1351 | CB | ASN B | 306 | 57.558 | 43.318 | 38.449 | 1.00 | 39.69 | B |
| ATOM | 1352 | CG | ASN B | 306 | 57.874 | 41.836 | 38.265 | 1.00 | 41.87 | B |
| ATOM | 1353 | OD1 | ASN B | 306 | 58.898 | 41.465 | 37.690 | 1.00 | 42.36 | B |
| ATOM | 1354 | ND2 | ASN B | 306 | 56.989 | 40.983 | 38.763 | 1.00 | 44.37 | B |
| ATOM | 1355 | C | ASN B | 306 | 59.730 | 43.817 | 39.608 | 1.00 | 35.41 | B |
| ATOM | 1356 | O | ASN B | 306 | 60.333 | 42.902 | 40.170 | 1.00 | 35.33 | B |
| ATOM | 1357 | N | LYS B | 307 | 60.353 | 44.765 | 38.917 | 1.00 | 34.02 | B |
| ATOM | 1358 | CA | LYS B | 307 | 61.804 | 44.750 | 38.786 | 1.00 | 34.18 | B |
| ATOM | 1359 | CB | LYS B | 307 | 62.245 | 45.720 | 37.686 | 1.00 | 34.16 | B |
| ATOM | 1360 | CG | LYS B | 307 | 63.753 | 45.889 | 37.572 | 1.00 | 34.46 | B |
| ATOM | 1361 | CD | LYS B | 307 | 64.121 | 46.934 | 36.524 | 1.00 | 36.66 | B |
| ATOM | 1362 | CE | LYS B | 307 | 63.783 | 46.479 | 35.089 | 1.00 | 39.17 | B |
| ATOM | 1363 | NZ | LYS B | 307 | 62.308 | 46.327 | 34.816 | 1.00 | 41.27 | B |
| ATOM | 1364 | C | LYS B | 307 | 62.491 | 45.096 | 40.110 | 1.00 | 34.15 | B |
| ATOM | 1365 | O | LYS B | 307 | 63.484 | 44.476 | 40.486 | 1.00 | 35.79 | B |
| ATOM | 1366 | N | ILE B | 308 | 61.967 | 46.089 | 40.814 | 1.00 | 33.56 | B |
| ATOM | 1367 | CA | ILE B | 308 | 62.539 | 46.483 | 42.090 | 1.00 | 32.11 | B |
| ATOM | 1368 | CB | ILE B | 308 | 61.790 | 47.706 | 42.670 | 1.00 | 31.25 | B |
| ATOM | 1369 | CG2 | ILE B | 308 | 62.235 | 47.954 | 44.118 | 1.00 | 29.28 | B |
| ATOM | 1370 | CG1 | ILE B | 308 | 62.010 | 48.918 | 41.746 | 1.00 | 30.74 | B |
| ATOM | 1371 | CD1 | ILE B | 308 | 61.163 | 50.148 | 42.057 | 1.00 | 30.81 | B |
| ATOM | 1372 | C | ILE B | 308 | 62.455 | 45.330 | 43.083 | 1.00 | 32.53 | B |
| ATOM | 1373 | O | ILE B | 308 | 63.432 | 44.996 | 43.741 | 1.00 | 33.24 | B |
| ATOM | 1374 | N | ILE B | 309 | 61.288 | 44.706 | 43.174 | 1.00 | 32.71 | B |
| ATOM | 1375 | CA | ILE B | 309 | 61.094 | 43.616 | 44.120 | 1.00 | 32.29 | B |
| ATOM | 1376 | CB | ILE B | 309 | 59.605 | 43.255 | 44.224 | 1.00 | 31.48 | B |
| ATOM | 1377 | CG2 | ILE B | 309 | 59.436 | 41.944 | 44.993 | 1.00 | 28.42 | B |
| ATOM | 1378 | CG1 | ILE B | 309 | 58.860 | 44.419 | 44.894 | 1.00 | 29.43 | B |
| ATOM | 1379 | CD1 | ILE B | 309 | 57.377 | 44.244 | 45.030 | 1.00 | 29.32 | B |
| ATOM | 1380 | C | ILE B | 309 | 61.881 | 42.360 | 43.801 | 1.00 | 32.95 | B |
| ATOM | 1381 | O | ILE B | 309 | 62.297 | 41.616 | 44.705 | 1.00 | 33.10 | B |
| ATOM | 1382 | N | ARG B | 310 | 62.088 | 42.120 | 42.515 | 1.00 | 32.48 | B |
| ATOM | 1383 | CA | ARG B | 310 | 62.807 | 40.929 | 42.113 | 1.00 | 31.41 | B |
| ATOM | 1384 | CB | ARG B | 310 | 62.401 | 40.562 | 40.701 | 1.00 | 30.53 | B |
| ATOM | 1385 | CG | ARG B | 310 | 61.047 | 39.954 | 40.749 | 1.00 | 32.30 | B |
| ATOM | 1386 | CD | ARG B | 310 | 60.974 | 38.823 | 39.812 | 1.00 | 33.94 | B |
| ATOM | 1387 | NE | ARG B | 310 | 60.659 | 39.310 | 38.485 | 1.00 | 36.53 | B |
| ATOM | 1388 | CZ | ARG B | 310 | 60.989 | 38.682 | 37.369 | 1.00 | 37.75 | B |
| ATOM | 1389 | NH1 | ARG B | 310 | 61.656 | 37.535 | 37.428 | 1.00 | 36.97 | B |
| ATOM | 1390 | NH2 | ARG B | 310 | 60.649 | 39.205 | 36.200 | 1.00 | 38.45 | B |
| ATOM | 1391 | C | ARG B | 310 | 64.302 | 41.065 | 42.259 | 1.00 | 30.16 | B |
| ATOM | 1392 | O | ARG B | 310 | 65.059 | 40.091 | 42.152 | 1.00 | 28.61 | B |
| ATOM | 1393 | N | LYS B | 311 | 64.717 | 42.283 | 42.552 | 1.00 | 29.95 | B |
| ATOM | 1394 | CA | LYS B | 311 | 66.122 | 42.542 | 42.727 | 1.00 | 31.78 | B |
| ATOM | 1395 | CB | LYS B | 311 | 66.419 | 44.014 | 42.471 | 1.00 | 30.98 | B |
| ATOM | 1396 | CG | LYS B | 311 | 67.876 | 44.355 | 42.676 | 1.00 | 34.65 | B |
| ATOM | 1397 | CD | LYS B | 311 | 68.141 | 45.839 | 42.539 | 0.00 | 34.39 | B |
| ATOM | 1398 | CE | LYS B | 311 | 68.033 | 46.305 | 41.095 | 0.00 | 35.04 | B |
| ATOM | 1399 | NZ | LYS B | 311 | 66.665 | 46.176 | 40.520 | 0.00 | 35.23 | B |
| ATOM | 1400 | C | LYS B | 311 | 66.589 | 42.154 | 44.127 | 1.00 | 31.89 | B |
| ATOM | 1401 | O | LYS B | 311 | 67.761 | 41.815 | 44.325 | 1.00 | 31.11 | B |
| ATOM | 1402 | N | SER B | 312 | 65.666 | 42.181 | 45.088 | 1.00 | 32.44 | B |
| ATOM | 1403 | CA | SER B | 312 | 65.997 | 41.868 | 46.479 | 1.00 | 34.40 | B |
| ATOM | 1404 | CB | SER B | 312 | 64.998 | 42.541 | 47.433 | 1.00 | 35.97 | B |
| ATOM | 1405 | OG | SER B | 312 | 65.318 | 42.268 | 48.793 | 1.00 | 37.24 | B |
| ATOM | 1406 | C | SER B | 312 | 66.068 | 40.389 | 46.825 | 1.00 | 33.58 | B |
| ATOM | 1407 | O | SER B | 312 | 65.154 | 39.629 | 46.532 | 1.00 | 35.14 | B |
| ATOM | 1408 | N | THR B | 313 | 67.160 | 39.990 | 47.460 | 1.00 | 32.17 | B |
| ATOM | 1409 | CA | THR B | 313 | 67.314 | 38.613 | 47.867 | 1.00 | 32.11 | B |
| ATOM | 1410 | CB | THR B | 313 | 68.760 | 38.142 | 47.682 | 1.00 | 34.02 | B |
| ATOM | 1411 | OG1 | THR B | 313 | 68.921 | 37.648 | 46.346 | 1.00 | 35.15 | B |
| ATOM | 1412 | CG2 | THR B | 313 | 69.114 | 37.055 | 48.685 | 1.00 | 33.55 | B |
| ATOM | 1413 | C | THR B | 313 | 66.913 | 38.532 | 49.329 | 1.00 | 30.50 | B |
| ATOM | 1414 | O | THR B | 313 | 66.318 | 37.553 | 49.764 | 1.00 | 31.15 | B |
| ATOM | 1415 | N | SER B | 314 | 67.219 | 39.583 | 50.078 | 1.00 | 29.28 | B |
| ATOM | 1416 | CA | SER B | 314 | 66.881 | 39.648 | 51.493 | 1.00 | 28.64 | B |
| ATOM | 1417 | CB | SER B | 314 | 67.307 | 40.990 | 52.076 | 1.00 | 28.48 | B |
| ATOM | 1418 | OG | SER B | 314 | 68.696 | 40.979 | 52.349 | 1.00 | 33.47 | B |
| ATOM | 1419 | C | SER B | 314 | 65.397 | 39.479 | 51.710 | 1.00 | 27.68 | B |
| ATOM | 1420 | O | SER B | 314 | 64.966 | 38.738 | 52.592 | 1.00 | 28.00 | B |
| ATOM | 1421 | N | LEU B | 315 | 64.629 | 40.182 | 50.890 | 1.00 | 25.95 | B |
| ATOM | 1422 | CA | LEU B | 315 | 63.190 | 40.158 | 50.967 | 1.00 | 24.83 | B |
| ATOM | 1423 | CB | LEU B | 315 | 62.614 | 40.721 | 49.676 | 1.00 | 22.38 | B |
| ATOM | 1424 | CG | LEU B | 315 | 61.151 | 41.129 | 49.740 | 1.00 | 21.41 | B |
| ATOM | 1425 | CD1 | LEU B | 315 | 61.006 | 42.400 | 50.604 | 1.00 | 20.01 | B |
| ATOM | 1426 | CD2 | LEU B | 315 | 60.662 | 41.376 | 48.345 | 1.00 | 19.89 | B |
| ATOM | 1427 | C | LEU B | 315 | 62.675 | 38.745 | 51.206 | 1.00 | 26.71 | B |
| ATOM | 1428 | O | LEU B | 315 | 61.894 | 38.494 | 52.141 | 1.00 | 27.54 | B |
| ATOM | 1429 | N | TRP B | 316 | 63.110 | 37.813 | 50.369 | 1.00 | 26.88 | B |
| ATOM | 1430 | CA | TRP B | 316 | 62.665 | 36.439 | 50.534 | 1.00 | 27.42 | B |
| ATOM | 1431 | CB | TRP B | 316 | 62.776 | 35.691 | 49.204 | 1.00 | 27.04 | B |
| ATOM | 1432 | CG | TRP B | 316 | 62.021 | 36.416 | 48.176 | 1.00 | 25.52 | B |
| ATOM | 1433 | CD2 | TRP B | 316 | 60.600 | 36.519 | 48.088 | 1.00 | 25.34 | B |
| ATOM | 1434 | CE2 | TRP B | 316 | 60.313 | 37.456 | 47.071 | 1.00 | 24.36 | B |
| ATOM | 1435 | CE3 | TRP B | 316 | 59.540 | 35.920 | 48.778 | 1.00 | 26.16 | B |
| ATOM | 1436 | CD1 | TRP B | 316 | 62.526 | 37.262 | 47.232 | 1.00 | 25.54 | B |
| ATOM | 1437 | NE1 | TRP B | 316 | 61.507 | 37.892 | 46.566 | 1.00 | 23.85 | B |
| ATOM | 1438 | CZ2 | TRP B | 316 | 59.003 | 37.808 | 46.728 | 1.00 | 23.50 | B |
| ATOM | 1439 | CZ3 | TRP B | 316 | 58.236 | 36.272 | 48.438 | 1.00 | 24.82 | B |
| ATOM | 1440 | CH2 | TRP B | 316 | 57.981 | 37.209 | 47.421 | 1.00 | 24.99 | B |
| ATOM | 1441 | C | TRP B | 316 | 63.402 | 35.720 | 51.656 | 1.00 | 27.65 | B |
| ATOM | 1442 | O | TRP B | 316 | 62.786 | 34.989 | 52.446 | 1.00 | 27.76 | B |
| ATOM | 1443 | N | LYS B | 317 | 64.709 | 35.934 | 51.744 | 1.00 | 27.10 | B |
| ATOM | 1444 | CA | LYS B | 317 | 65.455 | 35.304 | 52.817 | 1.00 | 28.92 | B |
| ATOM | 1445 | CB | LYS B | 317 | 66.907 | 35.788 | 52.830 | 1.00 | 30.51 | B |
| ATOM | 1446 | CG | LYS B | 317 | 67.768 | 35.175 | 53.935 | 1.00 | 32.68 | B |
| ATOM | 1447 | CD | LYS B | 317 | 69.209 | 35.655 | 53.820 | 1.00 | 35.89 | B |
| ATOM | 1448 | CE | LYS B | 317 | 69.849 | 35.839 | 55.186 | 1.00 | 37.32 | B |
| ATOM | 1449 | NZ | LYS B | 317 | 69.169 | 36.927 | 55.978 | 1.00 | 39.48 | B |
| ATOM | 1450 | C | LYS B | 317 | 64.764 | 35.597 | 54.164 | 1.00 | 29.21 | B |
| ATOM | 1451 | O | LYS B | 317 | 64.560 | 34.682 | 54.975 | 1.00 | 28.72 | B |
| ATOM | 1452 | N | LYS B | 318 | 64.373 | 36.851 | 54.394 | 1.00 | 27.41 | B |
| ATOM | 1453 | CA | LYS B | 318 | 63.698 | 37.182 | 55.640 | 1.00 | 27.23 | B |
| ATOM | 1454 | CB | LYS B | 318 | 63.287 | 38.657 | 55.685 | 1.00 | 28.22 | B |
| ATOM | 1455 | CG | LYS B | 318 | 64.448 | 39.580 | 55.982 | 1.00 | 29.33 | B |
| ATOM | 1456 | CD | LYS B | 318 | 64.011 | 40.989 | 56.263 | 1.00 | 29.29 | B |
| ATOM | 1457 | CE | LYS B | 318 | 65.211 | 41.805 | 56.726 | 1.00 | 31.20 | B |
| ATOM | 1458 | NZ | LYS B | 318 | 66.332 | 41.748 | 55.749 | 1.00 | 29.62 | B |
| ATOM | 1459 | C | LYS B | 318 | 62.475 | 36.318 | 55.846 | 1.00 | 28.19 | B |
| ATOM | 1460 | O | LYS B | 318 | 62.325 | 35.651 | 56.881 | 1.00 | 29.19 | B |
| ATOM | 1461 | N | LEU B | 319 | 61.598 | 36.346 | 54.847 | 1.00 | 28.31 | B |
| ATOM | 1462 | CA | LEU B | 319 | 60.353 | 35.576 | 54.851 | 1.00 | 26.08 | B |
| ATOM | 1463 | CB | LEU B | 319 | 59.644 | 35.756 | 53.499 | 1.00 | 24.60 | B |
| ATOM | 1464 | CG | LEU B | 319 | 58.949 | 37.106 | 53.299 | 1.00 | 23.31 | B |
| ATOM | 1465 | CD1 | LEU B | 319 | 58.929 | 37.501 | 51.828 | 1.00 | 25.42 | B |
| ATOM | 1466 | CD2 | LEU B | 319 | 57.550 | 37.030 | 53.849 | 1.00 | 20.66 | B |
| ATOM | 1467 | C | LEU B | 319 | 60.661 | 34.104 | 55.115 | 1.00 | 24.68 | B |
| ATOM | 1468 | O | LEU B | 319 | 60.126 | 33.511 | 56.051 | 1.00 | 24.33 | B |
| ATOM | 1469 | N | LEU B | 320 | 61.521 | 33.515 | 54.292 | 1.00 | 24.37 | B |
| ATOM | 1470 | CA | LEU B | 320 | 61.887 | 32.119 | 54.496 | 1.00 | 24.33 | B |
| ATOM | 1471 | CB | LEU B | 320 | 63.119 | 31.777 | 53.678 | 1.00 | 20.97 | B |
| ATOM | 1472 | CG | LEU B | 320 | 62.711 | 31.425 | 52.272 | 1.00 | 20.93 | B |
| ATOM | 1473 | CD1 | LEU B | 320 | 63.945 | 31.333 | 51.431 | 1.00 | 21.63 | B |
| ATOM | 1474 | CD2 | LEU B | 320 | 61.936 | 30.117 | 52.276 | 1.00 | 17.66 | B |
| ATOM | 1475 | C | LEU B | 320 | 62.166 | 31.800 | 55.967 | 1.00 | 24.55 | B |
| ATOM | 1476 | O | LEU B | 320 | 61.744 | 30.769 | 56.491 | 1.00 | 25.31 | B |
| ATOM | 1477 | N | ILE B | 321 | 62.883 | 32.706 | 56.615 | 1.00 | 24.47 | B |
| ATOM | 1478 | CA | ILE B | 321 | 63.253 | 32.579 | 58.009 | 1.00 | 23.96 | B |
| ATOM | 1479 | CB | ILE B | 321 | 64.450 | 33.531 | 58.315 | 1.00 | 25.52 | B |
| ATOM | 1480 | CG2 | ILE B | 321 | 64.759 | 33.556 | 59.806 | 1.00 | 25.19 | B |
| ATOM | 1481 | CG1 | ILE B | 321 | 65.671 | 33.094 | 57.498 | 1.00 | 23.92 | B |
| ATOM | 1482 | CD1 | ILE B | 321 | 66.951 | 33.873 | 57.782 | 1.00 | 24.41 | B |
| ATOM | 1483 | C | ILE B | 321 | 62.065 | 32.882 | 58.928 | 1.00 | 23.22 | B |
| ATOM | 1484 | O | ILE B | 321 | 61.902 | 32.240 | 59.952 | 1.00 | 24.18 | B |
| ATOM | 1485 | N | SER B | 322 | 61.239 | 33.853 | 58.563 | 1.00 | 23.04 | B |
| ATOM | 1486 | CA | SER B | 322 | 60.079 | 34.200 | 59.370 | 1.00 | 23.65 | B |
| ATOM | 1487 | CB | SER B | 322 | 59.248 | 35.266 | 58.670 | 1.00 | 21.62 | B |
| ATOM | 1488 | OG | SER B | 322 | 60.030 | 36.403 | 58.387 | 1.00 | 24.32 | B |
| ATOM | 1489 | C | SER B | 322 | 59.203 | 32.990 | 59.581 | 1.00 | 24.95 | B |
| ATOM | 1490 | O | SER B | 322 | 58.789 | 32.691 | 60.696 | 1.00 | 28.00 | B |
| ATOM | 1491 | N | GLD B | 323 | 58.926 | 32.302 | 58.485 | 1.00 | 25.30 | B |
| ATOM | 1492 | CA | GLD B | 323 | 58.066 | 31.135 | 58.486 | 1.00 | 25.97 | B |
| ATOM | 1493 | CB | GLD B | 323 | 57.480 | 30.974 | 57.091 | 1.00 | 26.35 | B |
| ATOM | 1494 | CG | GLD B | 323 | 56.792 | 32.239 | 56.646 | 1.00 | 27.43 | B |
| ATOM | 1495 | CD | GLD B | 323 | 55.441 | 32.431 | 57.307 | 1.00 | 27.80 B | |
| ATOM | 1496 | OE1 | GLD B | 323 | 55.239 | 31.878 | 58.409 | 1.00 | 25.90 | B |
| ATOM | 1497 | OE2 | GLD B | 323 | 54.590 | 33.142 | 56.718 | 1.00 | 28.13 | B |
| ATOM | 1498 | C | GLD B | 323 | 58.745 | 29.855 | 58.917 | 1.00 | 25.13 | B |
| ATOM | 1499 | O | GLD B | 323 | 58.123 | 28.792 | 58.931 | 1.00 | 25.20 | B |
| ATOM | 1500 | N | ASN B | 324 | 60.019 | 29.966 | 59.276 | 1.00 | 24.08 | B |
| ATOM | 1501 | CA | ASN B | 324 | 60.808 | 28.816 | 59.708 | 1.00 | 24.96 | B |
| ATOM | 1502 | CB | ASN B | 324 | 60.203 | 28.185 | 60.974 | 1.00 | 26.02 | B |
| ATOM | 1503 | CG | ASN B | 324 | 60.051 | 29.187 | 62.108 | 1.00 | 28.82 | B |
| ATOM | 1504 | OD1 | ASN B | 324 | 61.030 | 29.766 | 62.605 | 1.00 | 29.61 | B |
| ATOM | 1505 | ND2 | ASN B | 324 | 58.813 | 29.402 | 62.518 | 1.00 | 29.76 | B |
| ATOM | 1506 | C | ASN B | 324 | 60.906 | 27.776 | 58.588 | 1.00 | 24.92 | B |
| ATOM | 1507 | O | ASN B | 324 | 60.642 | 26.596 | 58.786 | 1.00 | 23.18 | B |
| ATOM | 1508 | N | PHE B | 325 | 61.269 | 28.226 | 57.395 | 1.00 | 25.71 | B |
| ATOM | 1509 | CA | PHE B | 325 | 61.406 | 27.304 | 56.287 | 1.00 | 25.91 | B |
| ATOM | 1510 | CB | PHE B | 325 | 60.839 | 27.913 | 55.008 | 1.00 | 24.25 | B |
| ATOM | 1511 | CG | PHE B | 325 | 59.334 | 27.935 | 54.987 | 1.00 | 23.73 | B |
| ATOM | 1512 | CD1 | PHE B | 325 | 58.644 | 28.984 | 54.394 | 1.00 | 23.04 | B |
| ATOM | 1513 | CD2 | PHE B | 325 | 58.604 | 26.925 | 55.616 | 1.00 | 20.21 | B |
| ATOM | 1514 | CE1 | PHE B | 325 | 57.259 | 29.025 | 54.437 | 1.00 | 20.96 | B |
| ATOM | 1515 | CE2 | PHE B | 325 | 57.227 | 26.964 | 55.658 | 1.00 | 18.74 | B |
| ATOM | 1516 | CZ | PHE B | 325 | 56.555 | 28.015 | 55.073 | 1.00 | 19.51 | B |
| ATOM | 1517 | C | PHE B | 325 | 62.861 | 26.972 | 56.156 | 1.00 | 26.92 | B |
| ATOM | 1518 | O | PHE B | 325 | 63.232 | 26.019 | 55.478 | 1.00 | 28.62 | B |
| ATOM | 1519 | N | VAL B | 326 | 63.683 | 27.766 | 56.833 | 1.00 | 27.50 | B |
| ATOM | 1520 | CA | VAL B | 326 | 65.127 | 27.577 | 56.849 | 1.00 | 27.18 | B |
| ATOM | 1521 | CB | VAL B | 326 | 65.773 | 28.060 | 55.532 | 1.00 | 25.75 | B |
| ATOM | 1522 | CG1 | VAL B | 326 | 66.019 | 29.553 | 55.587 | 1.00 | 23.02 | B |
| ATOM | 1523 | CG2 | VAL B | 326 | 67.061 | 27.316 | 55.288 | 1.00 | 26.04 | B |
| ATOM | 1524 | C | VAL B | 326 | 65.685 | 28.408 | 57.993 | 1.00 | 28.22 | B |
| ATOM | 1525 | O | VAL B | 326 | 64.998 | 29.274 | 58.533 | 1.00 | 28.23 | B |
| ATOM | 1526 | N | SER B | 327 | 66.932 | 28.154 | 58.361 | 1.00 | 29.87 | B |
| ATOM | 1527 | CA | SER B | 327 | 67.547 | 28.926 | 59.427 | 1.00 | 32.04 | B |
| ATOM | 1528 | CB | SER B | 327 | 67.867 | 28.053 | 60.643 | 1.00 | 32.92 | B |
| ATOM | 1529 | OG | SER B | 327 | 69.012 | 27.244 | 60.404 | 1.00 | 35.60 | B |
| ATOM | 1530 | C | SER B | 327 | 68.837 | 29.511 | 58.905 | 1.00 | 33.44 | B |
| ATOM | 1531 | O | SER B | 327 | 69.404 | 29.024 | 57.925 | 1.00 | 35.13 | B |
| ATOM | 1532 | N | PRO B | 328 | 69.328 | 30.561 | 59.568 | 1.00 | 34.95 | B |
| ATOM | 1533 | CD | PRO B | 328 | 68.703 | 31.277 | 60.698 | 1.00 | 34.72 | B |
| ATOM | 1534 | CA | PRO B | 328 | 70.570 | 31.208 | 59.161 | 1.00 | 35.43 | B |
| ATOM | 1535 | CB | PRO B | 328 | 70.906 | 32.056 | 60.372 | 1.00 | 34.44 | B |
| ATOM | 1536 | CG | PRO B | 328 | 69.541 | 32.530 | 60.792 | 1.00 | 34.00 | B |
| ATOM | 1537 | C | PRO B | 328 | 71.674 | 30.211 | 58.789 | 1.00 | 37.33 | B |
| ATOM | 1538 | O | PRO B | 328 | 72.413 | 30.428 | 57.820 | 1.00 | 38.07 | B |
| ATOM | 1539 | N | LYS B | 329 | 71.779 | 29.106 | 59.524 | 1.00 | 38.13 | B |
| ATOM | 1540 | CA | LYS B | 329 | 72.830 | 28.126 | 59.222 | 1.00 | 39.44 | B |
| ATOM | 1541 | CB | LYS B | 329 | 73.088 | 27.201 | 60.423 | 1.00 | 41.96 | B |
| ATOM | 1542 | CG | LYS B | 329 | 73.334 | 27.911 | 61.736 | 1.00 | 44.54 | B |
| ATOM | 1543 | CD | LYS B | 329 | 74.564 | 28.801 | 61.693 | 1.00 | 46.08 | B |
| ATOM | 1544 | CE | LYS B | 329 | 74.745 | 29.475 | 63.047 | 1.00 | 47.88 | B |
| ATOM | 1545 | NZ | LYS B | 329 | 73.510 | 30.241 | 63.384 | 1.00 | 48.71 | B |
| ATOM | 1546 | C | LYS B | 329 | 72.539 | 27.255 | 58.005 | 1.00 | 38.23 | B |
| ATOM | 1547 | O | LYS B | 329 | 73.444 | 26.922 | 57.230 | 1.00 | 37.43 | B |
| ATOM | 1548 | N | GLY B | 330 | 71.279 | 26.863 | 57.858 | 1.00 | 36.83 | B |
| ATOM | 1549 | CA | GLY B | 330 | 70.904 | 26.026 | 56.733 | 1.00 | 34.91 | B |
| ATOM | 1550 | C | GLY B | 330 | 70.671 | 26.809 | 55.458 | 1.00 | 33.34 | B |
| ATOM | 1551 | O | GLY B | 330 | 70.432 | 26.217 | 54.402 | 1.00 | 31.96 | B |
| ATOM | 1552 | N | PHE B | 331 | 70.742 | 28.136 | 55.554 | 1.00 | 32.27 | B |
| ATOM | 1553 | CA | PHE B | 331 | 70.517 | 28.977 | 54.388 | 1.00 | 32.81 | B |
| ATOM | 1554 | CB | PHE B | 331 | 70.778 | 30.454 | 54.688 | 1.00 | 32.71 | B |
| ATOM | 1555 | CG | PHE B | 331 | 70.365 | 31.374 | 53.561 | 1.00 | 35.02 | B |
| ATOM | 1556 | CD1 | PHE B | 331 | 69.013 | 31.631 | 53.319 | 1.00 | 34.88 | B |
| ATOM | 1557 | CD2 | PHE B | 331 | 71.323 | 31.971 | 52.727 | 1.00 | 36.79 | B |
| ATOM | 1558 | CE1 | PHE B | 331 | 68.609 | 32.466 | 52.274 | 1.00 | 35.03 | B |
| ATOM | 1559 | CE2 | PHE B | 331 | 70.933 | 32.816 | 51.670 | 1.00 | 36.82 | B |
| ATOM | 1560 | CZ | PHE B | 331 | 69.568 | 33.061 | 51.446 | 1.00 | 36.85 | B |
| ATOM | 1561 | C | PHE B | 331 | 71.333 | 28.600 | 53.164 | 1.00 | 32.72 | B |
| ATOM | 1562 | O | PHE B | 331 | 70.779 | 28.197 | 52.143 | 1.00 | 32.53 | B |
| ATOM | 1563 | N | ASN B | 332 | 72.650 | 28.725 | 53.261 | 1.00 | 33.63 | B |
| ATOM | 1564 | CA | ASN B | 332 | 73.495 | 28.430 | 52.116 | 1.00 | 34.96 | B |
| ATOM | 1565 | CB | ASN B | 332 | 74.956 | 28.510 | 52.503 | 1.00 | 35.07 | B |
| ATOM | 1566 | CG | ASN B | 332 | 75.386 | 29.925 | 52.769 | 1.00 | 36.45 | B |
| ATOM | 1567 | OD1 | ASN B | 332 | 74.860 | 30.864 | 52.165 | 1.00 | 36.09 | B |
| ATOM | 1568 | ND2 | ASN B | 332 | 76.352 | 30.096 | 53.666 | 1.00 | 37.34 | B |
| ATOM | 1569 | C | ASN B | 332 | 73.204 | 27.105 | 51.457 | 1.00 | 35.37 | B |
| ATOM | 1570 | O | ASN B | 332 | 73.006 | 27.034 | 50.240 | 1.00 | 34.82 | B |
| ATOM | 1571 | N | SER B | 333 | 73.174 | 26.050 | 52.253 | 1.00 | 36.35 | B |
| ATOM | 1572 | CA | SER B | 333 | 72.897 | 24.749 | 51.696 | 1.00 | 38.23 | B |
| ATOM | 1573 | CB | SER B | 333 | 72.767 | 23.724 | 52.804 | 1.00 | 37.83 | B |
| ATOM | 1574 | OG | SER B | 333 | 72.509 | 22.458 | 52.236 | 1.00 | 43.08 | B |
| ATOM | 1575 | C | SER B | 333 | 71.615 | 24.785 | 50.867 | 1.00 | 38.81 | B |
| ATOM | 1576 | O | SER B | 333 | 71.522 | 24.144 | 49.814 | 1.00 | 39.26 | B |
| ATOM | 1577 | N | LEU B | 334 | 70.629 | 25.533 | 51.357 | 1.00 | 38.86 | B |
| ATOM | 1578 | CA | LEU B | 334 | 69.338 | 25.688 | 50.685 | 1.00 | 37.16 | B |
| ATOM | 1579 | CB | LEU B | 334 | 68.446 | 26.645 | 51.486 | 1.00 | 34.21 | B |
| ATOM | 1580 | CG | LEU B | 334 | 67.149 | 27.126 | 50.830 | 1.00 | 33.30 | B |
| ATOM | 1581 | CD1 | LEU B | 334 | 66.189 | 25.957 | 50.642 | 1.00 | 29.33 | B |
| ATOM | 1582 | CD2 | LEU B | 334 | 66.528 | 28.209 | 51.688 | 1.00 | 31.65 | B |
| ATOM | 1583 | C | LEU B | 334 | 69.574 | 26.251 | 49.291 | 1.00 | 37.44 | B |
| ATOM | 1584 | O | LEU B | 334 | 69.105 | 25.700 | 48.293 | 1.00 | 36.40 | B |
| ATOM | 1585 | N | ASN B | 335 | 70.306 | 27.361 | 49.248 | 1.00 | 38.10 | B |
| ATOM | 1586 | CA | ASN B | 335 | 70.647 | 28.032 | 48.001 | 1.00 | 38.02 | B |
| ATOM | 1587 | CB | ASN B | 335 | 71.590 | 29.188 | 48.289 | 1.00 | 37.88 | B |
| ATOM | 1588 | CG | ASN B | 335 | 70.871 | 30.496 | 48.329 | 1.00 | 40.34 | B |
| ATOM | 1589 | OD1 | ASN B | 335 | 71.266 | 31.422 | 49.033 | 1.00 | 43.76 | B |
| ATOM | 1590 | ND2 | ASN B | 335 | 69.800 | 30.591 | 47.556 | 1.00 | 41.28 | B |
| ATOM | 1591 | C | ASN B | 335 | 71.292 | 27.070 | 47.013 | 1.00 | 38.24 | B |
| ATOM | 1592 | O | ASN B | 335 | 70.906 | 27.003 | 45.847 | 1.00 | 38.64 | B |
| ATOM | 1593 | N | LEU B | 336 | 72.274 | 26.325 | 47.498 | 1.00 | 36.78 | B |
| ATOM | 1594 | CA | LEU B | 336 | 72.978 | 25.364 | 46.683 | 1.00 | 35.71 | B |
| ATOM | 1595 | CB | LEU B | 336 | 74.041 | 24.658 | 47.522 | 1.00 | 35.78 | B |
| ATOM | 1596 | CG | LEU B | 336 | 74.991 | 23.682 | 46.827 | 1.00 | 35.54 | B |
| ATOM | 1597 | CD1 | LEU B | 336 | 75.977 | 24.436 | 45.942 | 1.00 | 35.55 | B |
| ATOM | 1598 | CD2 | LEU B | 336 | 75.716 | 22.899 | 47.883 | 1.00 | 36.31 | B |
| ATOM | 1599 | C | LEU B | 336 | 71.997 | 24.336 | 46.123 | 1.00 | 35.77 | B |
| ATOM | 1600 | O | LEU B | 336 | 72.075 | 23.961 | 44.954 | 1.00 | 37.71 | B |
| ATOM | 1601 | N | LYS B | 337 | 71.068 | 23.876 | 46.953 | 1.00 | 34.49 | B |
| ATOM | 1602 | CA | LYS B | 337 | 70.097 | 22.878 | 46.508 | 1.00 | 31.85 | B |
| ATOM | 1603 | CB | LYS B | 337 | 69.310 | 22.320 | 47.704 | 1.00 | 30.25 | B |
| ATOM | 1604 | CG | LYS B | 337 | 68.298 | 21.237 | 47.321 | 1.00 | 32.14 | B |
| ATOM | 1605 | CD | LYS B | 337 | 67.438 | 20.697 | 48.512 | 1.00 | 35.02 | B |
| ATOM | 1606 | CE | LYS B | 337 | 66.544 | 21.789 | 49.173 | 1.00 | 35.41 | B |
| ATOM | 1607 | NZ | LYS B | 337 | 65.466 | 21.260 | 50.081 | 1.00 | 32.74 | B |
| ATOM | 1608 | C | LYS B | 337 | 69.129 | 23.433 | 45.461 | 1.00 | 30.95 | B |
| ATOM | 1609 | O | LYS B | 337 | 68.793 | 22.740 | 44.519 | 1.00 | 29.84 | B |
| ATOM | 1610 | N | LEU B | 338 | 68.690 | 24.679 | 45.636 | 1.00 | 31.30 | B |
| ATOM | 1611 | CA | LEU B | 338 | 67.755 | 25.324 | 44.714 | 1.00 | 30.02 | B |
| ATOM | 1612 | CB | LEU B | 338 | 67.218 | 26.621 | 45.317 | 1.00 | 29.04 | B |
| ATOM | 1613 | CG | LEU B | 338 | 66.157 | 26.563 | 46.434 | 1.00 | 29.18 | B |
| ATOM | 1614 | CD1 | LEU B | 338 | 65.834 | 27.996 | 46.909 | 1.00 | 25.48 | B |
| ATOM | 1615 | CD2 | LEU B | 338 | 64.897 | 25.881 | 45.919 | 1.00 | 26.03 | B |
| ATOM | 1616 | C | LEU B | 338 | 68.433 | 25.639 | 43.400 | 1.00 | 30.20 | B |
| ATOM | 1617 | O | LEU B | 338 | 67.809 | 25.643 | 42.340 | 1.00 | 29.78 | B |
| ATOM | 1618 | N | SER B | 339 | 69.724 | 25.918 | 43.487 | 1.00 | 30.16 | B |
| ATOM | 1619 | CA | SER B | 339 | 70.510 | 26.227 | 42.317 | 1.00 | 29.06 | B |
| ATOM | 1620 | CB | SER B | 339 | 71.913 | 26.656 | 42.712 | 1.00 | 27.05 | B |
| ATOM | 1621 | OG | SER B | 339 | 72.665 | 26.965 | 41.560 | 1.00 | 25.97 | B |
| ATOM | 1622 | C | SER B | 339 | 70.598 | 24.979 | 41.483 | 1.00 | 30.90 | B |
| ATOM | 1623 | O | SER B | 339 | 70.580 | 25.029 | 40.254 | 1.00 | 32.89 | B |
| ATOM | 1624 | N | GLN B | 340 | 70.701 | 23.842 | 42.149 | 1.00 | 30.57 | B |
| ATOM | 1625 | CA | GLN B | 340 | 70.796 | 22.605 | 41.409 | 1.00 | 31.13 | B |
| ATOM | 1626 | CB | GLN B | 340 | 71.238 | 21.496 | 42.334 | 1.00 | 32.53 | B |
| ATOM | 1627 | CG | GLN B | 340 | 72.619 | 21.768 | 42.867 | 1.00 | 35.89 | B |
| ATOM | 1628 | CD | GLN B | 340 | 73.040 | 20.758 | 43.900 | 1.00 | 36.30 | B |
| ATOM | 1629 | OE1 | GLN B | 340 | 72.209 | 19.975 | 44.393 | 1.00 | 37.07 | B |
| ATOM | 1630 | NE2 | GLN B | 340 | 74.332 | 20.765 | 44.248 | 1.00 | 34.68 | B |
| ATOM | 1631 | C | GLN B | 340 | 69.471 | 22.276 | 40.774 | 1.00 | 31.71 | B |
| ATOM | 1632 | O | GLN B | 340 | 69.418 | 21.644 | 39.726 | 1.00 | 32.80 | B |
| ATOM | 1633 | N | LYS B | 341 | 68.403 | 22.738 | 41.408 | 1.00 | 32.39 | B |
| ATOM | 1634 | CA | LYS B | 341 | 67.047 | 22.505 | 40.942 | 1.00 | 33.18 | B |
| ATOM | 1635 | CB | LYS B | 341 | 66.086 | 22.760 | 42.094 | 1.00 | 34.86 | B |
| ATOM | 1636 | CG | LYS B | 341 | 64.697 | 22.223 | 41.889 | 1.00 | 38.21 | B |
| ATOM | 1637 | CD | LYS B | 341 | 63.793 | 22.574 | 43.061 | 1.00 | 40.03 | B |
| ATOM | 1638 | CE | LYS B | 341 | 62.406 | 21.980 | 42.836 | 1.00 | 42.58 | B |
| ATOM | 1639 | NZ | LYS B | 341 | 61.380 | 22.609 | 43.719 | 1.00 | 42.83 | B |
| ATOM | 1640 | C | LYS B | 341 | 66.661 | 23.392 | 39.764 | 1.00 | 34.36 | B |
| ATOM | 1641 | O | LYS B | 341 | 66.041 | 22.939 | 38.805 | 1.00 | 34.14 | B |
| ATOM | 1642 | N | TYR B | 342 | 67.043 | 24.658 | 39.857 | 1.00 | 34.90 | B |
| ATOM | 1643 | CA | TYR B | 342 | 66.734 | 25.660 | 38.853 | 1.00 | 35.01 | B |
| ATOM | 1644 | CB | TYR B | 342 | 65.844 | 26.695 | 39.497 | 1.00 | 37.87 | B |
| ATOM | 1645 | CG | TYR B | 342 | 64.647 | 26.082 | 40.153 | 1.00 | 41.65 | B |
| ATOM | 1646 | CD1 | TYR B | 342 | 63.646 | 25.507 | 39.384 | 1.00 | 42.60 | B |
| ATOM | 1647 | CE1 | TYR B | 342 | 62.525 | 24.958 | 39.968 | 1.00 | 45.40 | B |
| ATOM | 1648 | CD2 | TYR B | 342 | 64.501 | 26.088 | 41.538 | 1.00 | 42.64 | B |
| ATOM | 1649 | CE2 | TYR B | 342 | 63.376 | 25.540 | 42.139 | 1.00 | 44.31 | B |
| ATOM | 1650 | CZ | TYR B | 342 | 62.386 | 24.978 | 41.343 | 1.00 | 45.73 | B |
| ATOM | 1651 | OH | TYR B | 342 | 61.232 | 24.462 | 41.896 | 1.00 | 46.50 | B |
| ATOM | 1652 | C | TYR B | 342 | 67.991 | 26.339 | 38.343 | 1.00 | 34.71 | B |
| ATOM | 1653 | O | TYR B | 342 | 68.287 | 27.467 | 38.729 | 1.00 | 35.57 | B |
| ATOM | 1654 | N | PRO B | 343 | 68.754 | 25.663 | 37.474 | 1.00 | 34.09 | B |
| ATOM | 1655 | CD | PRO B | 343 | 68.568 | 24.298 | 36.955 | 1.00 | 33.06 | B |
| ATOM | 1656 | CA | PRO B | 343 | 69.982 | 26.256 | 36.942 | 1.00 | 34.15 | B |
| ATOM | 1657 | CB | PRO B | 343 | 70.427 | 25.231 | 35.902 | 1.00 | 32.61 | B |
| ATOM | 1658 | CG | PRO B | 343 | 69.940 | 23.953 | 36.458 | 1.00 | 30.81 | B |
| ATOM | 1659 | C | PRO B | 343 | 69.805 | 27.655 | 36.332 | 1.00 | 34.54 | B |
| ATOM | 1660 | O | PRO B | 343 | 70.562 | 28.577 | 36.639 | 1.00 | 34.01 | B |
| ATOM | 1661 | N | LYS B | 344 | 68.793 | 27.803 | 35.483 | 1.00 | 34.28 | B |
| ATOM | 1662 | CA | LYS B | 344 | 68.537 | 29.063 | 34.799 | 1.00 | 34.41 | B |
| ATOM | 1663 | CB | LYS B | 344 | 67.823 | 28.791 | 33.483 | 1.00 | 34.70 | B |
| ATOM | 1664 | CG | LYS B | 344 | 68.487 | 27.758 | 32.623 | 1.00 | 32.84 | B |
| ATOM | 1665 | CD | LYS B | 344 | 67.566 | 27.409 | 31.505 | 1.00 | 32.13 | B |
| ATOM | 1666 | CE | LYS B | 344 | 67.769 | 25.984 | 31.078 | 1.00 | 32.95 | B |
| ATOM | 1667 | NZ | LYS B | 344 | 66.694 | 25.581 | 30.144 | 1.00 | 35.30 | B |
| ATOM | 1668 | C | LYS B | 344 | 67.778 | 30.156 | 35.547 | 1.00 | 34.52 | B |
| ATOM | 1669 | CD | LYS B | 344 | 67.446 | 31.188 | 34.950 | 1.00 | 33.51 | B |
| ATOM | 1670 | N | LEU B | 345 | 67.475 | 29.921 | 36.825 | 1.00 | 32.93 | B |
| ATOM | 1671 | CA | LEU B | 345 | 66.792 | 30.916 | 37.663 | 1.00 | 30.85 | B |
| ATOM | 1672 | CB | LEU B | 345 | 65.856 | 30.253 | 38.660 | 1.00 | 28.33 | B |
| ATOM | 1673 | CG | LEU B | 345 | 64.506 | 29.850 | 38.111 | 1.00 | 27.26 | B |
| ATOM | 1674 | CD1 | LEU B | 345 | 63.691 | 29.121 | 39.179 | 1.00 | 24.62 | B |
| ATOM | 1675 | CD2 | LEU B | 345 | 63.804 | 31.102 | 37.655 | 1.00 | 26.44 | B |
| ATOM | 1676 | C | LEU B | 345 | 67.834 | 31.674 | 38.440 | 1.00 | 31.07 | B |
| ATOM | 1677 | O | LEU B | 345 | 68.961 | 31.210 | 38.580 | 1.00 | 31.79 | B |
| ATOM | 1678 | N | SER B | 346 | 67.458 | 32.830 | 38.970 | 1.00 | 33.11 | B |
| ATOM | 1679 | CA | SER B | 346 | 68.392 | 33.653 | 39.754 | 1.00 | 34.10 | B |
| ATOM | 1680 | CB | SER B | 346 | 68.283 | 35.114 | 39.349 | 1.00 | 34.20 | B |
| ATOM | 1681 | OG | SER B | 346 | 67.035 | 35.623 | 39.779 | 1.00 | 37.09 | B |
| ATOM | 1682 | C | SER B | 346 | 68.098 | 33.563 | 41.247 | 1.00 | 33.12 | B |
| ATOM | 1683 | O | SER B | 346 | 66.971 | 33.302 | 41.655 | 1.00 | 33.78 | B |
| ATOM | 1684 | N | GLN B | 347 | 69.113 | 33.814 | 42.056 | 1.00 | 32.28 | B |
| ATOM | 1685 | CA | GLN B | 347 | 68.950 | 33.749 | 43.495 | 1.00 | 32.88 | B |
| ATOM | 1686 | CB | GLN B | 347 | 70.071 | 34.502 | 44.177 | 1.00 | 33.73 | B |
| ATOM | 1687 | CG | GLN B | 347 | 70.009 | 34.451 | 45.667 | 1.00 | 33.40 | B |
| ATOM | 1688 | CD | GLN B | 347 | 71.105 | 35.276 | 46.251 | 1.00 | 36.19 | B |
| ATOM | 1689 | OE1 | GLN B | 347 | 71.164 | 36.494 | 46.031 | 1.00 | 38.25 | B |
| ATOM | 1690 | NE2 | GLN B | 347 | 72.006 | 34.627 | 46.984 | 1.00 | 36.38 | B |
| ATOM | 1691 | C | GLN B | 347 | 67.620 | 34.314 | 43.973 | 1.00 | 31.82 | B |
| ATOM | 1692 | O | GLN B | 347 | 66.834 | 33.616 | 44.620 | 1.00 | 31.26 | B |
| ATOM | 1693 | N | GLN B | 348 | 67.370 | 35.581 | 43.661 | 1.00 | 30.61 | B |
| ATOM | 1694 | CA | GLN B | 348 | 66.121 | 36.195 | 44.076 | 1.00 | 30.85 | B |
| ATOM | 1695 | CB | GLN B | 348 | 65.984 | 37.626 | 43.542 | 1.00 | 31.61 | B |
| ATOM | 1696 | CG | GLN B | 348 | 67.092 | 38.583 | 43.958 | 1.00 | 31.41 | B |
| ATOM | 1697 | CD | GLN B | 348 | 68.291 | 38.552 | 43.014 | 1.00 | 30.33 | B |
| ATOM | 1698 | OE1 | GLN B | 348 | 69.055 | 39.512 | 42.942 | 1.00 | 29.04 | B |
| ATOM | 1699 | NE2 | GLN B | 348 | 68.461 | 37.452 | 42.296 | 1.00 | 29.86 | B |
| ATOM | 1700 | C | GLN B | 348 | 64.942 | 35.364 | 43.593 | 1.00 | 29.45 | B |
| ATOM | 1701 | O | GLN B | 348 | 64.183 | 34.831 | 44.400 | 1.00 | 28.92 | B |
| ATOM | 1702 | N | ASP B | 349 | 64.776 | 35.236 | 42.283 | 1.00 | 28.60 | B |
| ATOM | 1703 | CA | ASP B | 349 | 63.646 | 34.458 | 41.805 | 1.00 | 27.39 | B |
| ATOM | 1704 | CB | ASP B | 349 | 63.631 | 34.372 | 40.282 | 1.00 | 28.50 | B |
| ATOM | 1705 | CG | ASP B | 349 | 63.164 | 35.666 | 39.632 | 1.00 | 30.39 | B |
| ATOM | 1706 | OD1 | ASP B | 349 | 62.212 | 36.306 | 40.153 | 1.00 | 29.22 | B |
| ATOM | 1707 | OD2 | ASP B | 349 | 63.744 | 36.034 | 38.590 | 1.00 | 31.83 | B |
| ATOM | 1708 | C | ASP B | 349 | 63.571 | 33.061 | 42.404 | 1.00 | 25.37 | B |
| ATOM | 1709 | O | ASP B | 349 | 62.476 | 32.546 | 42.597 | 1.00 | 24.91 | B |
| ATOM | 1710 | N | ARG B | 350 | 64.720 | 32.454 | 42.695 | 1.00 | 22.81 | B |
| ATOM | 1711 | CA | ARG B | 350 | 64.746 | 31.117 | 43.280 | 1.00 | 21.59 | B |
| ATOM | 1712 | CB | ARG B | 350 | 66.178 | 30.611 | 43.428 | 1.00 | 23.28 | B |
| ATOM | 1713 | CG | ARG B | 350 | 66.787 | 30.106 | 42.133 | 1.00 | 27.24 | B |
| ATOM | 1714 | CD | ARG B | 350 | 68.195 | 29.544 | 42.333 | 1.00 | 26.74 | B |
| ATOM | 1715 | NE | ARG B | 350 | 68.820 | 29.271 | 41.051 | 1.00 | 27.71 | B |
| ATOM | 1716 | CZ | ARG B | 350 | 70.128 | 29.218 | 40.860 | 1.00 | 29.79 | B |
| ATOM | 1717 | NH1 | ARG B | 350 | 70.956 | 29.416 | 41.878 | 1.00 | 31.36 | B |
| ATOM | 1718 | NH2 | ARG B | 350 | 70.604 | 28.990 | 39.642 | 1.00 | 31.57 | B |
| ATOM | 1719 | C | ARG B | 350 | 64.094 | 31.124 | 44.651 | 1.00 | 20.94 | B |
| ATOM | 1720 | O | ARG B | 350 | 63.103 | 30.429 | 44.893 | 1.00 | 20.47 | B |
| ATOM | 1721 | N | LEU B | 351 | 64.672 | 31.909 | 45.549 | 1.00 | 18.79 | B |
| ATOM | 1722 | CA | LEU B | 351 | 64.157 | 32.026 | 46.890 | 1.00 | 17.44 | B |
| ATOM | 1723 | CB | LEU B | 351 | 64.895 | 33.143 | 47.624 | 1.00 | 14.47 | B |
| ATOM | 1724 | CG | LEU B | 351 | 66.358 | 32.799 | 47.897 | 1.00 | 14.78 | B |
| ATOM | 1725 | CD1 | LEU B | 351 | 66.992 | 33.900 | 48.730 | 1.00 | 17.09 | B |
| ATOM | 1726 | CD2 | LEU B | 351 | 66.466 | 31.467 | 48.619 | 1.00 | 12.98 | B |
| ATOM | 1727 | C | LEU B | 351 | 62.638 | 32.247 | 46.942 | 1.00 | 18.50 | B |
| ATOM | 1728 | O | LEU B | 351 | 61.958 | 31.715 | 47.826 | 1.00 | 17.10 | B |
| ATOM | 1729 | N | ARG B | 352 | 62.092 | 33.003 | 45.995 | 1.00 | 18.20 | B |
| ATOM | 1730 | CA | ARG B | 352 | 60.652 | 33.246 | 45.997 | 1.00 | 20.70 | B |
| ATOM | 1731 | CB | ARG B | 352 | 60.301 | 34.404 | 45.060 | 1.00 | 22.90 | B |
| ATOM | 1732 | CG | ARG B | 352 | 58.821 | 34.519 | 44.704 | 1.00 | 21.25 | B |
| ATOM | 1733 | CD | ARG B | 352 | 58.577 | 35.861 | 44.037 | 1.00 | 24.96 | B |
| ATOM | 1734 | NE | ARG B | 352 | 59.336 | 36.027 | 42.793 | 1.00 | 26.60 | B |
| ATOM | 1735 | CZ | ARG B | 352 | 58.840 | 35.772 | 41.588 | 1.00 | 24.08 | B |
| ATOM | 1736 | NH1 | ARG B | 352 | 57.589 | 35.344 | 41.468 | 1.00 | 25.95 | B |
| ATOM | 1737 | NH2 | ARG B | 352 | 59.582 | 35.955 | 40.508 | 1.00 | 23.94 | B |
| ATOM | 1738 | C | ARG B | 352 | 59.868 | 32.014 | 45.590 | 1.00 | 20.80 | B |
| ATOM | 1739 | O | ARG B | 352 | 58.812 | 31.701 | 46.151 | 1.00 | 20.69 | B |
| ATOM | 1740 | N | LEU B | 353 | 60.385 | 31.308 | 44.601 | 1.00 | 22.07 | B |
| ATOM | 1741 | CA | LEU B | 353 | 59.703 | 30.115 | 44.135 | 1.00 | 24.32 | B |
| ATOM | 1742 | CB | LEU B | 353 | 60.494 | 29.510 | 42.971 | 1.00 | 24.67 | B |
| ATOM | 1743 | CG | LEU B | 353 | 59.774 | 28.524 | 42.058 | 1.00 | 26.68 | B |
| ATOM | 1744 | CO1 | LEU B | 353 | 59.623 | 27.208 | 42.767 | 1.00 | 28.84 | B |
| ATOM | 1745 | CD2 | LEU B | 353 | 58.420 | 29.098 | 41.635 | 1.00 | 28.75 | B |
| ATOM | 1746 | C | LEU B | 353 | 59.614 | 29.166 | 45.336 | 1.00 | 25.14 | B |
| ATOM | 1747 | O | LEU B | 353 | 58.568 | 28.566 | 45.604 | 1.00 | 24.06 | B |
| ATOM | 1748 | N | SER B | 354 | 60.728 | 29.085 | 46.063 | 1.00 | 25.53 | B |
| ATOM | 1749 | CA | SER B | 354 | 60.867 | 28.264 | 47.262 | 1.00 | 26.14 | B |
| ATOM | 1750 | CB | SER B | 354 | 62.273 | 28.450 | 47.849 | 1.00 | 25.96 | B |
| ATOM | 1751 | OG | SER B | 354 | 62.454 | 27.730 | 49.061 | 1.00 | 23.97 | B |
| ATOM | 1752 | C | SER B | 354 | 59.830 | 28.656 | 48.313 | 1.00 | 27.19 | B |
| ATOM | 1753 | O | SER B | 354 | 59.098 | 27.818 | 48.837 | 1.00 | 27.24 | B |
| ATOM | 1754 | N | PHE B | 355 | 59.769 | 29.941 | 48.625 | 1.00 | 27.04 | B |
| ATOM | 1755 | CA | PHE B | 355 | 58.822 | 30.390 | 49.616 | 1.00 | 26.23 | B |
| ATOM | 1756 | CB | PHE B | 355 | 58.954 | 31.882 | 49.854 | 1.00 | 27.13 | B |
| ATOM | 1757 | CG | PHE B | 355 | 58.041 | 32.400 | 50.920 | 1.00 | 26.97 | B |
| ATOM | 1758 | CD1 | PHE B | 355 | 56.736 | 32.763 | 50.626 | 1.00 | 26.60 | B |
| ATOM | 1759 | CD2 | PHE B | 355 | 58.495 | 32.522 | 52.228 | 1.00 | 28.32 | B |
| ATOM | 1760 | CE1 | PHE B | 355 | 55.892 | 33.236 | 51.626 | 1.00 | 27.60 | B |
| ATOM | 1761 | CE2 | PHE B | 355 | 57.661 | 32.992 | 53.237 | 1.00 | 26.91 | B |
| ATOM | 1762 | CZ | PHE B | 355 | 56.360 | 33.353 | 52.934 | 1.00 | 27.81 | B |
| ATOM | 1763 | C | PHE B | 355 | 57.413 | 30.087 | 49.203 | 1.00 | 25.95 | B |
| ATOM | 1764 | O | PHE B | 355 | 56.687 | 29.427 | 49.929 | 1.00 | 25.86 | B |
| ATOM | 1765 | N | LEU B | 356 | 57.015 | 30.575 | 48.037 | 1.00 | 27.00 | B |
| ATOM | 1766 | CA | LEU B | 356 | 55.655 | 30.337 | 47.570 | 1.00 | 27.63 | B |
| ATOM | 1767 | CB | LEU B | 356 | 55.487 | 30.886 | 46.144 | 1.00 | 28.43 | B |
| ATOM | 1768 | CG | LEU B | 356 | 55.485 | 32.412 | 45.923 | 1.00 | 28.92 | B |
| ATOM | 1769 | CD1 | LEU B | 356 | 55.449 | 32.704 | 44.421 | 1.00 | 28.25 | B |
| ATOM | 1770 | CD2 | LEU B | 356 | 54.299 | 33.063 | 46.620 | 1.00 | 26.99 | B |
| ATOM | 1771 | C | LEU B | 356 | 55.297 | 28.842 | 47.618 | 1.00 | 26.51 | B |
| ATOM | 1772 | O | LEU B | 356 | 54.170 | 28.492 | 47.910 | 1.00 | 25.42 | B |
| ATOM | 1773 | N | GLU B | 357 | 56.267 | 27.976 | 47.343 | 1.00 | 27.82 | B |
| ATOM | 1774 | CA | GLU B | 357 | 56.051 | 26.537 | 47.357 | 1.00 | 28.89 | B |
| ATOM | 1775 | CB | GLU B | 357 | 57.223 | 25.841 | 46.668 | 1.00 | 30.18 | B |
| ATOM | 1776 | CG | GLU B | 357 | 57.193 | 24.319 | 46.727 | 1.00 | 34.82 | B |
| ATOM | 1777 | CD | GLU B | 357 | 58.032 | 23.659 | 45.640 | 1.00 | 38.48 | B |
| ATOM | 1778 | OE1 | GLU B | 357 | 59.254 | 23.926 | 45.569 | 1.00 | 41.06 | B |
| ATOM | 1779 | OE2 | GLU B | 357 | 57.460 | 22.870 | 44.849 | 1.00 | 40.28 | B |
| ATOM | 1780 | C | GLU B | 357 | 55.899 | 26.016 | 48.790 | 1.00 | 29.56 | B |
| ATOM | 1781 | O | GLU B | 357 | 55.055 | 25.165 | 49.072 | 1.00 | 30.56 | B |
| ATOM | 1782 | N | ASN B | 358 | 56.722 | 26.529 | 49.692 | 1.00 | 27.38 | B |
| ATOM | 1783 | CA | ASN B | 358 | 56.659 | 26.124 | 51.072 | 1.00 | 26.16 | B |
| ATOM | 1784 | CB | ASN B | 358 | 57.913 | 26.583 | 51.804 | 1.00 | 24.91 | B |
| ATOM | 1785 | CG | ASN B | 358 | 58.964 | 25.505 | 51.866 | 1.00 | 24.24 | B |
| ATOM | 1786 | OD1 | ASN B | 358 | 60.149 | 25.794 | 51.827 | 1.00 | 24.48 | B |
| ATOM | 1787 | ND2 | ASN B | 358 | 58.530 | 24.242 | 51.976 | 1.00 | 23.61 | B |
| ATOM | 1788 | C | ASN B | 358 | 55.440 | 26.669 | 51.796 | 1.00 | 27.01 | B |
| ATOM | 1789 | O | ASN B | 358 | 54.743 | 25.940 | 52.500 | 1.00 | 29.45 | B |
| ATOM | 1790 | N | ILE B | 359 | 55.169 | 27.948 | 51.603 | 1.00 | 25.54 | B |
| ATOM | 1791 | CA | ILE B | 359 | 54.066 | 28.576 | 52.287 | 1.00 | 24.86 | B |
| ATOM | 1792 | CB | ILE B | 359 | 53.989 | 30.111 | 51.964 | 1.00 | 23.57 | B |
| ATOM | 1793 | CG2 | ILE B | 359 | 53.438 | 30.361 | 50.544 | 1.00 | 22.38 | B |
| ATOM | 1794 | CG1 | ILE B | 359 | 53.099 | 30.808 | 52.994 | 1.00 | 21.88 | B |
| ATOM | 1795 | CD1 | ILE B | 359 | 53.627 | 30.735 | 54.428 | 1.00 | 17.84 | B |
| ATOM | 1796 | C | ILE B | 359 | 52.726 | 27.929 | 51.992 | 1.00 | 27.18 | B |
| ATOM | 1797 | O | ILE B | 359 | 51.864 | 27.842 | 52.859 | 1.00 | 27.37 | B |
| ATOM | 1798 | N | PHE B | 360 | 52.533 | 27.457 | 50.775 | 1.00 | 29.04 | B |
| ATOM | 1799 | CA | PHE B | 360 | 51.260 | 26.850 | 50.480 | 1.00 | 30.13 | B |
| ATOM | 1800 | CB | PHE B | 360 | 50.977 | 26.994 | 48.976 | 1.00 | 31.09 | B |
| ATOM | 1801 | CG | PHE B | 360 | 50.729 | 28.441 | 48.552 | 1.00 | 35.13 | B |
| ATOM | 1802 | CD1 | PHE B | 360 | 49.750 | 29.214 | 49.196 | 1.00 | 36.04 | B |
| ATOM | 1803 | CD2 | PHE B | 360 | 51.480 | 29.040 | 47.540 | 1.00 | 35.99 | B |
| ATOM | 1804 | CE1 | PHE B | 360 | 49.531 | 30.554 | 48.837 | 1.00 | 36.47 | B |
| ATOM | 1805 | CE2 | PHE B | 360 | 51.265 | 30.380 | 47.177 | 1.00 | 37.16 | B |
| ATOM | 1806 | CZ | PHE B | 360 | 50.290 | 31.138 | 47.827 | 1.00 | 35.98 | B |
| ATOM | 1807 | C | PHE B | 360 | 51.137 | 25.414 | 51.019 | 1.00 | 28.89 | B |
| ATOM | 1808 | O | PHE B | 360 | 50.040 | 24.932 | 51.282 | 1.00 | 29.91 | B |
| ATOM | 1809 | N | ILE B | 361 | 52.258 | 24.739 | 51.221 | 1.00 | 26.91 | B |
| ATOM | 1810 | CA | ILE B | 361 | 52.212 | 23.405 | 51.799 | 1.00 | 25.90 | B |
| ATOM | 1811 | CE | ILE B | 361 | 53.567 | 22.687 | 51.626 | 1.00 | 24.75 | B |
| ATOM | 1812 | CG2 | ILE B | 361 | 53.707 | 21.556 | 52.648 | 1.00 | 20.19 | B |
| ATOM | 1813 | CG1 | ILE B | 361 | 53.695 | 22.198 | 50.187 | 1.00 | 21.38 | B |
| ATOM | 1814 | CD1 | ILE B | 361 | 55.017 | 21.579 | 49.880 | 1.00 | 21.49 | B |
| ATOM | 1815 | C | ILE B | 361 | 51.894 | 23.539 | 53.303 | 1.00 | 25.76 | B |
| ATOM | 1816 | O | ILE B | 361 | 51.165 | 22.724 | 53.854 | 1.00 | 25.25 | B |
| ATOM | 1817 | N | LEU B | 362 | 52.455 | 24.558 | 53.955 | 1.00 | 24.78 | B |
| ATOM | 1818 | CA | LEU B | 362 | 52.200 | 24.780 | 55.367 | 1.00 | 24.80 | B |
| ATOM | 1819 | CB | LEU B | 362 | 53.097 | 25.890 | 55.942 | 1.00 | 24.95 | B |
| ATOM | 1820 | CG | LEU B | 362 | 52.861 | 26.232 | 57.439 | 1.00 | 25.61 | B |
| ATOM | 1821 | CD1 | LEU B | 362 | 53.251 | 25.034 | 58.290 | 1.00 | 23.05 | B |
| ATOM | 1822 | CD2 | LEU B | 362 | 53.682 | 27.448 | 57.891 | 1.00 | 24.66 | B |
| ATOM | 1823 | C | LEU B | 362 | 50.746 | 25.192 | 55.479 | 1.00 | 24.68 | B |
| ATOM | 1824 | O | LEU B | 362 | 50.075 | 24.847 | 56.440 | 1.00 | 25.14 | B |
| ATOM | 1825 | N | LYS B | 363 | 50.262 | 25.932 | 54.485 | 1.00 | 26.90 | B |
| ATOM | 1826 | CA | LYS B | 363 | 48.870 | 26.397 | 54.455 | 1.00 | 28.18 | B |
| ATOM | 1827 | CB | LYS B | 363 | 48.607 | 27.192 | 53.178 | 1.00 | 30.22 | B |
| ATOM | 1828 | CG | LYS B | 363 | 48.946 | 28.677 | 53.262 | 1.00 | 34.21 | B |
| ATOM | 1829 | CD | LYS B | 363 | 47.787 | 29.466 | 53.860 | 1.00 | 35.29 | B |
| ATOM | 1830 | CE | LYS B | 363 | 48.036 | 30.984 | 53.859 | 1.00 | 38.15 | B |
| ATOM | 1831 | NZ | LYS B | 363 | 48.196 | 31.608 | 52.486 | 1.00 | 38.16 | B |
| ATOM | 1832 | C | LYS B | 363 | 47.931 | 25.209 | 54.497 | 1.00 | 27.14 | B |
| ATOM | 1833 | O | LYS B | 363 | 46.852 | 25.288 | 55.076 | 1.00 | 27.91 | B |
| ATOM | 1834 | N | ASN B | 364 | 48.365 | 24.119 | 53.872 | 1.00 | 26.50 | B |
| ATOM | 1835 | CA | ASN B | 364 | 47.602 | 22.893 | 53.809 | 1.00 | 28.02 | B |
| ATOM | 1836 | CB | ASN B | 364 | 48.119 | 21.990 | 52.692 | 1.00 | 29.30 | B |
| ATOM | 1837 | CG | ASN B | 364 | 47.742 | 22.485 | 51.311 | 1.00 | 30.37 | B |
| ATOM | 1838 | OD1 | ASN B | 364 | 48.127 | 21.882 | 50.299 | 1.00 | 29.53 | B |
| ATOM | 1839 | ND2 | ASN B | 364 | 46.982 | 23.584 | 51.255 | 1.00 | 31.60 | B |
| ATOM | 1840 | C | ASN B | 364 | 47.696 | 22.129 | 55.106 | 1.00 | 27.69 | B |
| ATOM | 1841 | O | ASN B | 364 | 46.716 | 21.551 | 55.559 | 1.00 | 28.47 | B |
| ATOM | 1842 | N | TRP B | 365 | 48.882 | 22.093 | 55.693 | 1.00 | 26.51 | B |
| ATOM | 1843 | CA | TRP B | 365 | 49.039 | 21.371 | 56.938 | 1.00 | 27.73 | B |
| ATOM | 1844 | CB | TRP B | 365 | 50.489 | 21.397 | 57.400 | 1.00 | 29.20 | B |
| ATOM | 1845 | CG | TRP B | 365 | 51.351 | 20.381 | 56.790 | 1.00 | 29.13 | B |
| ATOM | 1846 | CD2 | TRP B | 365 | 51.742 | 19.142 | 57.379 | 1.00 | 29.19 | B |
| ATOM | 1847 | CE2 | TRP B | 365 | 52.638 | 18.523 | 56.489 | 1.00 | 28.54 | B |
| ATOM | 1848 | CE3 | TRP B | 365 | 51.425 | 18.495 | 58.579 | 1.00 | 29.88 | B |
| ATOM | 1849 | CD1 | TRP B | 365 | 51.989 | 20.460 | 55.598 | 1.00 | 28.45 | B |
| ATOM | 1850 | NE1 | TRP B | 365 | 52.771 | 19.351 | 55.406 | 1.00 | 27.81 | B |
| ATOM | 1851 | CZ2 | TRP B | 365 | 53.230 | 17.282 | 56.758 | 1.00 | 28.80 | B |
| ATOM | 1852 | CZ3 | TRP B | 365 | 52.018 | 17.257 | 58.842 | 1.00 | 29.52 | B |
| ATOM | 1853 | CH2 | TRP B | 365 | 52.909 | 16.671 | 57.934 | 1.00 | 27.59 | B |
| ATOM | 1854 | C | TRP B | 365 | 48.183 | 21.976 | 58.037 | 1.00 | 27.25 | B |
| ATOM | 1855 | O | TRP B | 365 | 47.686 | 21.262 | 58.898 | 1.00 | 27.54 | B |
| ATOM | 1856 | N | TYR B | 366 | 48.015 | 23.293 | 57.985 | 1.00 | 25.59 | B |
| ATOM | 1857 | CA | TYR B | 366 | 47.271 | 24.038 | 58.989 | 1.00 | 25.83 | B |
| ATOM | 1858 | CB | TYR B | 366 | 47.858 | 25.433 | 59.127 | 1.00 | 24.61 | B |
| ATOM | 1859 | CG | TYR B | 366 | 49.220 | 25.559 | 59.761 | 1.00 | 22.97 | B |
| ATOM | 1860 | CD1 | TYR B | 366 | 49.803 | 24.511 | 60.469 | 1.00 | 23.61 | B |
| ATOM | 1861 | CE1 | TYR B | 366 | 50.999 | 24.699 | 61.170 | 1.00 | 21.04 | B |
| ATOM | 1862 | CD2 | TYR B | 366 | 49.871 | 26.779 | 59.760 | 1.00 | 21.89 | B |
| ATOM | 1863 | CE2 | TYR B | 366 | 51.050 | 26.968 | 60.452 | 1.00 | 20.89 | B |
| ATOM | 1864 | CZ | TYR B | 366 | 51.603 | 25.940 | 61.153 | 1.00 | 20.92 | B |
| ATOM | 1865 | OH | TYR B | 366 | 52.741 | 26.179 | 61.878 | 1.00 | 21.34 | B |
| ATOM | 1866 | C | TYR B | 366 | 45.781 | 24.205 | 58.730 | 1.00 | 26.75 | B |
| ATOM | 1867 | O | TYR B | 366 | 45.053 | 24.659 | 59.618 | 1.00 | 25.47 | B |
| ATOM | 1868 | N | ASN B | 367 | 45.343 | 23.874 | 57.516 | 1.00 | 27.78 | B |
| ATOM | 1869 | CA | ASN B | 367 | 43.935 | 23.993 | 57.137 | 1.00 | 28.96 | B |
| ATOM | 1870 | CB | ASN B | 367 | 43.789 | 24.062 | 55.611 | 1.00 | 27.81 | B |
| ATOM | 1871 | CG | ASN B | 367 | 42.420 | 24.542 | 55.172 | 1.00 | 27.70 | B |
| ATOM | 1872 | OD1 | ASN B | 367 | 41.462 | 24.533 | 55.944 | 1.00 | 29.38 | B |
| ATOM | 1873 | ND2 | ASN B | 367 | 42.321 | 24.957 | 53.915 | 1.00 | 27.06 | B |
| ATOM | 1874 | C | ASN B | 367 | 43.184 | 22.772 | 57.668 | 1.00 | 29.83 | B |
| ATOM | 1875 | O | ASN B | 367 | 43.454 | 21.638 | 57.253 | 1.00 | 30.52 | B |
| ATOM | 1876 | N | PRO B | 368 | 42.246 | 22.988 | 58.613 | 1.00 | 30.10 | B |
| ATOM | 1877 | CD | PRO B | 368 | 41.884 | 24.278 | 59.227 | 1.00 | 28.86 | B |
| ATOM | 1878 | CA | PRO B | 368 | 41.458 | 21.889 | 59.200 | 1.00 | 29.62 | B |
| ATOM | 1879 | CB | PRO B | 368 | 40.654 | 22.578 | 60.304 | 1.00 | 28.09 | B |
| ATOM | 1880 | CG | PRO B | 368 | 41.398 | 23.839 | 60.573 | 1.00 | 28.33 | B |
| ATOM | 1881 | C | PRO B | 368 | 40.534 | 21.318 | 58.133 | 1.00 | 29.34 | B |
| ATOM | 1882 | O | PRO B | 368 | 40.223 | 20.133 | 58.118 | 1.00 | 28.44 | B |
| ATOM | 1883 | N | LYS B | 369 | 40.083 | 22.202 | 57.256 | 1.00 | 29.08 | B |
| ATOM | 1884 | CA | LYS B | 369 | 39.202 | 21.814 | 56.187 | 1.00 | 30.72 | B |
| ATOM | 1885 | CB | LYS B | 369 | 38.417 | 23.044 | 55.697 | 1.00 | 32.78 | B |
| ATOM | 1886 | CG | LYS B | 369 | 37.095 | 23.310 | 56.487 | 1.00 | 34.07 | B |
| ATOM | 1887 | CD | LYS B | 369 | 36.649 | 24.778 | 56.462 | 1.00 | 36.76 | B |
| ATOM | 1888 | CE | LYS B | 369 | 36.882 | 25.442 | 55.098 | 1.00 | 38.43 | B |
| ATOM | 1889 | NZ | LYS B | 369 | 36.568 | 26.906 | 55.151 | 1.00 | 41.21 | B |
| ATOM | 1890 | C | LYS B | 369 | 39.977 | 21.145 | 55.050 | 1.00 | 30.60 | B |
| ATOM | 1891 | O | LYS B | 369 | 39.389 | 20.779 | 54.040 | 1.00 | 32.44 | B |
| ATOM | 1892 | N | PHE B | 370 | 41.293 | 20.985 | 55.207 | 1.00 | 29.96 | B |
| ATOM | 1893 | CA | PHE B | 370 | 42.111 | 20.335 | 54.175 | 1.00 | 28.15 | B |
| ATOM | 1894 | CB | PHE B | 370 | 43.422 | 21.107 | 53.934 | 1.00 | 29.31 | B |
| ATOM | 1895 | CG | PHE B | 370 | 44.447 | 20.330 | 53.124 | 1.00 | 31.81 | B |
| ATOM | 1896 | CD1 | PHE B | 370 | 45.283 | 19.394 | 53.738 | 1.00 | 34.71 | B |
| ATOM | 1897 | CD2 | PHE B | 370 | 44.536 | 20.484 | 51.745 | 1.00 | 31.92 | B |
| ATOM | 1898 | CE1 | PHE B | 370 | 46.185 | 18.625 | 52.992 | 1.00 | 34.80 | B |
| ATOM | 1899 | CE2 | PHE B | 370 | 45.436 | 19.718 | 50.992 | 1.00 | 33.21 | B |
| ATOM | 1900 | CZ | PHE B | 370 | 46.259 | 18.785 | 51.622 | 1.00 | 33.35 | B |
| ATOM | 1901 | C | PHE B | 370 | 42.431 | 18.886 | 54.537 | 1.00 | 26.13 | B |
| ATOM | 1902 | O | PHE B | 370 | 42.750 | 18.584 | 55.682 | 1.00 | 26.19 | B |
| ATOM | 1903 | N | VAL B | 371 | 42.352 | 17.993 | 53.556 | 1.00 | 25.26 | B |
| ATOM | 1904 | CA | VAL B | 371 | 42.656 | 16.590 | 53.811 | 1.00 | 24.76 | B |
| ATOM | 1905 | CB | VAL B | 371 | 41.419 | 15.694 | 53.697 | 1.00 | 25.22 | B |
| ATOM | 1906 | CG1 | VAL B | 371 | 41.736 | 14.297 | 54.237 | 1.00 | 22.91 | B |
| ATOM | 1907 | CG2 | VAL B | 371 | 40.257 | 16.324 | 54.442 | 1.00 | 23.49 | B |
| ATOM | 1908 | C | VAL B | 371 | 43.712 | 16.062 | 52.863 | 1.00 | 24.14 | B |
| ATOM | 1909 | O | VAL B | 371 | 43.585 | 16.188 | 51.653 | 1.00 | 23.89 | B |
| ATOM | 1910 | N | PRO B | 372 | 44.767 | 15.447 | 53.418 | 1.00 | 24.76 | B |
| ATOM | 1911 | CD | PRO B | 372 | 44.861 | 15.087 | 54.840 | 1.00 | 25.65 | B |
| ATOM | 1912 | CA | PRO B | 372 | 45.889 | 14.878 | 52.678 | 1.00 | 25.57 | B |
| ATOM | 1913 | CB | PRO B | 372 | 46.835 | 14.390 | 53.769 | 1.00 | 25.32 | B |
| ATOM | 1914 | CG | PRO B | 372 | 46.318 | 14.998 | 55.041 | 1.00 | 26.54 | B |
| ATOM | 1915 | C | PRO B | 372 | 45.421 | 13.720 | 51.863 | 1.00 | 27.92 | B |
| ATOM | 1916 | O | PRO B | 372 | 44.359 | 13.144 | 52.143 | 1.00 | 29.78 | B |
| ATOM | 1917 | N | GLN B | 373 | 46.223 | 13.375 | 50.858 | 1.00 | 28.98 | B |
| ATOM | 1918 | CA | GLN B | 373 | 45.940 | 12.222 | 50.002 | 1.00 | 29.41 | B |
| ATOM | 1919 | CB | GLN B | 373 | 46.566 | 12.412 | 48.600 | 1.00 | 29.51 | B |
| ATOM | 1920 | CG | GLN B | 373 | 45.660 | 13.145 | 47.586 | 1.00 | 29.73 | B |
| ATOM | 1921 | CD | GLN B | 373 | 46.354 | 13.455 | 46.247 | 1.00 | 30.26 | B |
| ATOM | 1922 | OE1 | GLN B | 373 | 47.091 | 14.429 | 46.129 | 1.00 | 30.70 | B |
| ATOM | 1923 | NE2 | GLN B | 373 | 46.113 | 12.621 | 45.240 | 1.00 | 30.61 | B |
| ATOM | 1924 | C | GLN B | 373 | 46.554 | 11.029 | 50.758 | 1.00 | 28.67 | B |
| ATOM | 1925 | O | GLN B | 373 | 47.662 | 11.119 | 51.274 | 1.00 | 28.24 | B |
| ATOM | 1926 | N | ARG B | 374 | 45.803 | 9.936 | 50.845 | 1.00 | 28.06 | B |
| ATOM | 1927 | CA | ARG B | 374 | 46.213 | 8.728 | 51.557 | 1.00 | 25.68 | B |
| ATOM | 1928 | CB | ARG B | 374 | 45.030 | 8.254 | 52.416 | 1.00 | 25.68 | B |
| ATOM | 1929 | CG | ARG B | 374 | 45.173 | 6.929 | 53.159 | 1.00 | 24.87 | B |
| ATOM | 1930 | CD | ARG B | 374 | 45.727 | 7.099 | 54.544 | 1.00 | 25.14 | B |
| ATOM | 1931 | NE | ARG B | 374 | 45.604 | 5.872 | 55.347 | 1.00 | 26.59 | B |
| ATOM | 1932 | CZ | ARG B | 374 | 44.549 | 5.554 | 56.099 | 1.00 | 24.91 | B |
| ATOM | 1933 | NH1 | ARG B | 374 | 43.503 | 6.370 | 56.171 | 1.00 | 23.91 | B |
| ATOM | 1934 | NH2 | ARG B | 374 | 44.533 | 4.407 | 56.770 | 1.00 | 23.39 | B |
| ATOM | 1935 | C | ARG B | 374 | 46.650 | 7.619 | 50.598 | 1.00 | 25.60 | B |
| ATOM | 1936 | O | ARG B | 374 | 45.978 | 7.352 | 49.591 | 1.00 | 25.61 | B |
| ATOM | 1937 | N | THR B | 375 | 47.790 | 7.000 | 50.909 | 1.00 | 24.83 | B |
| ATOM | 1938 | CA | THR B | 375 | 48.325 | 5.895 | 50.124 | 1.00 | 24.29 | B |
| ATOM | 1939 | CB | THR B | 375 | 49.562 | 6.298 | 49.278 | 1.00 | 24.62 | B |
| ATOM | 1940 | OG1 | THR B | 375 | 49.334 | 7.559 | 48.630 | 1.00 | 26.27 | B |
| ATOM | 1941 | CG2 | THR B | 375 | 49.845 | 5.228 | 48.217 | 1.00 | 22.20 | B |
| ATOM | 1942 | C | THR B | 375 | 48.787 | 4.844 | 51.125 | 1.00 | 25.05 | B |
| ATOM | 1943 | O | THR B | 375 | 49.710 | 5.108 | 51.889 | 1.00 | 25.56 | B |
| ATOM | 1944 | N | THR B | 376 | 48.149 | 3.669 | 51.122 | 1.00 | 25.33 | B |
| ATOM | 1945 | CA | THR B | 376 | 48.509 | 2.571 | 52.034 | 1.00 | 25.61 | B |
| ATOM | 1946 | CB | THR B | 376 | 47.254 | 1.951 | 52.733 | 1.00 | 23.00 | B |
| ATOM | 1947 | OG1 | THR B | 376 | 46.361 | 2.992 | 53.148 | 1.00 | 23.90 | B |
| ATOM | 1948 | CG2 | THR B | 376 | 47.678 | 1.125 | 53.956 | 1.00 | 21.63 | B |
| ATOM | 1949 | C | THR B | 376 | 49.224 | 1.439 | 51.273 | 1.00 | 27.50 | B |
| ATOM | 1950 | O | THR B | 376 | 48.696 | 0.916 | 50.303 | 1.00 | 27.69 | B |
| ATOM | 1951 | N | LEU B | 377 | 50.413 | 1.058 | 51.735 | 1.00 | 29.38 | B |
| ATOM | 1952 | CA | LEU B | 377 | 51.204 | −0.001 | 51.119 | 1.00 | 29.82 | B |
| ATOM | 1953 | CB | LEU B | 377 | 52.540 | 0.574 | 50.650 | 1.00 | 27.89 | B |
| ATOM | 1954 | CG | LEU B | 377 | 52.396 | 1.871 | 49.837 | 1.00 | 27.64 | B |
| ATOM | 1955 | CD1 | LEU B | 377 | 53.754 | 2.507 | 49.549 | 1.00 | 23.10 | B |
| ATOM | 1956 | CD2 | LEU B | 377 | 51.643 | 1.570 | 48.562 | 1.00 | 26.07 | B |
| ATOM | 1957 | C | LEU B | 377 | 51.451 | −1.131 | 52.125 | 1.00 | 32.73 | B |
| ATOM | 1958 | O | LEU B | 377 | 51.328 | −0.925 | 53.327 | 1.00 | 32.89 | B |
| ATOM | 1959 | N | ARG B | 378 | 51.815 | −2.315 | 51.633 | 1.00 | 36.51 | B |
| ATOM | 1960 | CA | ARG B | 378 | 52.071 | −3.463 | 52.498 | 1.00 | 39.20 | B |
| ATOM | 1961 | CB | ARG B | 378 | 52.180 | −4.757 | 51.678 | 1.00 | 42.06 | B |
| ATOM | 1962 | CG | ARG B | 378 | 52.510 | −6.019 | 52.506 | 1.00 | 45.42 | B |
| ATOM | 1963 | CD | ARG B | 378 | 52.472 | −7.335 | 51.682 | 1.00 | 48.17 | B |
| ATOM | 1964 | NE | ARG B | 378 | 51.210 | −7.513 | 50.958 | 1.00 | 51.30 | B |
| ATOM | 1965 | CZ | ARG B | 378 | 50.850 | −8.616 | 50.306 | 1.00 | 52.70 | B |
| ATOM | 1966 | NH1 | ARG B | 378 | 51.655 | −9.673 | 50.281 | 1.00 | 53.25 | B |
| ATOM | 1967 | NH2 | ARG B | 378 | 49.684 | −8.654 | 49.664 | 1.00 | 54.26 | B |
| ATOM | 1968 | C | ARG B | 378 | 53.333 | −3.278 | 53.307 | 1.00 | 40.74 | B |
| ATOM | 1969 | O | ARG B | 378 | 54.339 | −2.747 | 52.819 | 1.00 | 40.15 | B |
| ATOM | 1970 | N | GLY B | 379 | 53.266 | −3.735 | 54.555 | 1.00 | 42.99 | B |
| ATOM | 1971 | CA | GLY B | 379 | 54.394 | −3.628 | 55.463 | 1.00 | 43.31 | B |
| ATOM | 1972 | C | GLY B | 379 | 55.221 | −4.897 | 55.532 | 1.00 | 43.80 | B |
| ATOM | 1973 | O | GLY B | 379 | 55.501 | −5.532 | 54.516 | 1.00 | 42.41 | B |
| ATOM | 1974 | N | HIS B | 380 | 55.609 | −5.264 | 56.747 | 1.00 | 44.62 | B |
| ATOM | 1975 | CA | HIS B | 380 | 56.421 | −6.451 | 56.972 | 1.00 | 45.57 | B |
| ATOM | 1976 | CB | HIS B | 380 | 57.468 | −6.163 | 58.049 | 1.00 | 42.95 | B |
| ATOM | 1977 | CG | HIS B | 380 | 58.489 | −5.148 | 57.640 | 1.00 | 41.43 | B |
| ATOM | 1978 | CD2 | HIS B | 380 | 58.369 | −3.842 | 57.307 | 1.00 | 39.61 | B |
| ATOM | 1979 | ND1 | HIS B | 380 | 59.833 | −5.444 | 57.559 | 1.00 | 40.42 | B |
| ATOM | 1980 | CE1 | HIS B | 380 | 60.497 | −4.362 | 57.195 | 1.00 | 39.52 | B |
| ATOM | 1981 | NE2 | HIS B | 380 | 59.633 | −3.376 | 57.037 | 1.00 | 39.67 | B |
| ATOM | 1982 | C | HIS B | 380 | 55.552 | −7.627 | 57.398 | 1.00 | 47.13 | B |
| ATOM | 1983 | O | HIS B | 380 | 54.374 | −7.457 | 57.690 | 1.00 | 47.01 | B |
| ATOM | 1984 | N | MSE B | 381 | 56.127 | −8.822 | 57.440 | 1.00 | 49.63 | B |
| ATOM | 1985 | CA | MSE B | 381 | 55.350 | −9.979 | 57.826 | 1.00 | 51.34 | B |
| ATOM | 1986 | CB | MSE B | 381 | 56.181 | −11.238 | 57.658 | 1.00 | 54.42 | B |
| ATOM | 1987 | CG | MSE B | 381 | 56.131 | −11.740 | 56.225 | 1.00 | 60.08 | B |
| ATOM | 1988 | SE | MSE B | 381 | 57.583 | −12.923 | 55.737 | 1.00 | 69.56 | B |
| ATOM | 1989 | CE | MSE B | 381 | 56.901 | −14.517 | 56.629 | 1.00 | 64.33 | B |
| ATOM | 1990 | C | MSE B | 381 | 54.800 | −9.860 | 59.227 | 1.00 | 51.47 | B |
| ATOM | 1991 | O | MSE B | 381 | 53.645 | −10.193 | 59.464 | 1.00 | 53.52 | B |
| ATOM | 1992 | N | THR B | 382 | 55.608 | −9.366 | 60.158 | 1.00 | 50.41 | B |
| ATOM | 1993 | CA | THR B | 382 | 55.159 | −9.204 | 61.539 | 1.00 | 48.29 | B |
| ATOM | 1994 | CB | THR B | 382 | 56.367 | −9.222 | 62.513 | 1.00 | 48.92 | B |
| ATOM | 1995 | OG1 | THR B | 382 | 55.931 | −8.858 | 63.825 | 1.00 | 49.73 | B |
| ATOM | 1996 | CG2 | THR B | 382 | 57.457 | −8.259 | 62.048 | 1.00 | 51.19 | B |
| ATOM | 1997 | C | THR B | 382 | 54.361 | −7.903 | 61.674 | 1.00 | 46.73 | B |
| ATOM | 1998 | O | THR B | 382 | 54.511 | −6.990 | 60.858 | 1.00 | 45.72 | B |
| ATOM | 1999 | N | SER B | 383 | 53.510 | −7.818 | 62.695 | 1.00 | 45.75 | B |
| ATOM | 2000 | CA | SER B | 383 | 52.667 | −6.631 | 62.888 | 1.00 | 44.00 | B |
| ATOM | 2001 | CB | SER B | 383 | 51.418 | −6.988 | 63.706 | 1.00 | 44.17 | B |
| ATOM | 2002 | OG | SER B | 383 | 51.707 | −6.998 | 65.093 | 1.00 | 44.67 | B |
| ATOM | 2003 | C | SER B | 383 | 53.363 | −5.440 | 63.552 | 1.00 | 42.52 | B |
| ATOM | 2004 | O | SER B | 383 | 52.739 | −4.400 | 63.769 | 1.00 | 42.16 | B |
| ATOM | 2005 | N | VAL B | 384 | 54.645 | −5.584 | 63.875 | 1.00 | 40.61 | B |
| ATOM | 2006 | CA | VAL B | 384 | 55.368 | −4.496 | 64.519 | 1.00 | 38.88 | B |
| ATOM | 2007 | CB | VAL B | 384 | 55.785 | −4.868 | 65.971 | 1.00 | 37.56 | B |
| ATOM | 2008 | CG1 | VAL B | 384 | 56.490 | −3.706 | 66.635 | 1.00 | 34.97 | B |
| ATOM | 2009 | CG2 | VAL B | 384 | 54.570 | −5.273 | 66.772 | 1.00 | 37.83 | B |
| ATOM | 2010 | C | VAL B | 384 | 56.617 | −4.090 | 63.756 | 1.00 | 38.38 | B |
| ATOM | 2011 | O | VAL B | 384 | 57.507 | −4.906 | 63.506 | 1.00 | 38.18 | B |
| ATOM | 2012 | N | ILE B | 385 | 56.674 | −2.823 | 63.372 | 1.00 | 37.92 | B |
| ATOM | 2013 | CA | ILE B | 385 | 57.842 | −2.330 | 62.674 | 1.00 | 38.47 | B |
| ATOM | 2014 | CB | ILE B | 385 | 57.462 | −1.284 | 61.570 | 1.00 | 38.37 | B |
| ATOM | 2015 | CG2 | ILE B | 385 | 58.692 | −0.795 | 60.853 | 1.00 | 35.43 | B |
| ATOM | 2016 | CG1 | ILE B | 385 | 56.610 | −1.951 | 60.483 | 1.00 | 38.29 | B |
| ATOM | 2017 | CD1 | ILE B | 385 | 56.296 | −1.048 | 59.293 | 1.00 | 40.13 | B |
| ATOM | 2018 | C | ILE B | 385 | 58.752 | −1.744 | 63.758 | 1.00 | 38.62 | B |
| ATOM | 2019 | O | ILE B | 385 | 58.295 | −1.054 | 64.679 | 1.00 | 39.76 | B |
| ATOM | 2020 | N | THR B | 386 | 60.040 | −2.057 | 63.649 | 1.00 | 37.77 | B |
| ATOM | 2021 | CA | THR B | 386 | 61.053 | −1.643 | 64.616 | 1.00 | 34.37 | B |
| ATOM | 2022 | CB | THP. B | 386 | 62.142 | −2.722 | 64.734 | 1.00 | 32.81 | B |
| ATOM | 2023 | OG1 | THR B | 386 | 62.810 | −2.855 | 63.473 | 1.00 | 31.89 | B |
| ATOM | 2024 | CG2 | THR B | 386 | 61.528 | −4.050 | 65.098 | 1.00 | 32.45 | B |
| ATOM | 2025 | C | THR B | 386 | 61.740 | −0.329 | 64.281 | 1.00 | 32.70 | B |
| ATOM | 2026 | O | THR B | 386 | 62.088 | 0.438 | 65.167 | 1.00 | 33.06 | B |
| ATOM | 2027 | N | CYS B | 387 | 61.947 | −0.058 | 63.005 | 1.00 | 31.07 | B |
| ATOM | 2028 | CA | CYS B | 387 | 62.627 | 1.178 | 62.659 | 1.00 | 30.70 | B |
| ATOM | 2029 | CB | CYS B | 387 | 64.140 | 0.964 | 62.769 | 1.00 | 29.49 | B |
| ATOM | 2030 | SG | CYS B | 387 | 64.707 | −0.568 | 62.008 | 1.00 | 32.59 | B |
| ATOM | 2031 | C | CYS B | 387 | 62.250 | 1.702 | 61.268 | 1.00 | 29.94 | B |
| ATOM | 2032 | O | CYS B | 387 | 61.896 | 0.927 | 60.365 | 1.00 | 27.25 | B |
| ATOM | 2033 | N | LEU B | 388 | 62.349 | 3.021 | 61.097 | 1.00 | 29.32 | B |
| ATOM | 2034 | CA | LEU B | 388 | 62.001 | 3.659 | 59.828 | 1.00 | 28.34 | B |
| ATOM | 2035 | CB | LEU B | 388 | 60.534 | 4.097 | 59.878 | 1.00 | 28.15 | B |
| ATOM | 2036 | CG | LEU B | 388 | 59.848 | 4.716 | 58.662 | 1.00 | 25.84 | B |
| ATOM | 2037 | CD1 | LEU B | 388 | 58.358 | 4.729 | 58.925 | 1.00 | 26.04 | B |
| ATOM | 2038 | CD2 | LEU B | 388 | 60.361 | 6.117 | 58.401 | 1.00 | 24.86 | B |
| ATOM | 2039 | C | LEU B | 388 | 62.885 | 4.851 | 59.479 | 1.00 | 26.75 | B |
| ATOM | 2040 | O | LEU B | 388 | 63.211 | 5.661 | 60.336 | 1.00 | 25.66 | B |
| ATOM | 2041 | N | GLN B | 389 | 63.284 | 4.947 | 58.214 | 1.00 | 27.43 | B |
| ATOM | 2042 | CA | GLN B | 389 | 64.112 | 6.067 | 57.753 | 1.00 | 27.85 | B |
| ATOM | 2043 | CB | GLN B | 389 | 65.505 | 5.589 | 57.308 | 1.00 | 28.80 | B |
| ATOM | 2044 | CG | GLN B | 389 | 66.450 | 5.215 | 58.443 | 1.00 | 29.07 | B |
| ATOM | 2045 | CD | GLN B | 389 | 66.900 | 6.425 | 59.249 | 1.00 | 29.82 | B |
| ATOM | 2046 | OE1 | GLN B | 389 | 67.815 | 7.152 | 58.849 | 1.00 | 30.35 | B |
| ATOM | 2047 | NE2 | GLN B | 389 | 66.244 | 6.654 | 60.383 | 1.00 | 27.50 | B |
| ATOM | 2048 | C | GLN B | 389 | 63.417 | 6.718 | 56.577 | 1.00 | 26.75 | B |
| ATOM | 2049 | O | GLN B | 389 | 62.792 | 6.035 | 55.766 | 1.00 | 26.92 | B |
| ATOM | 2050 | N | PHE B | 390 | 63.532 | 8.037 | 56.483 | 1.00 | 26.42 | B |
| ATOM | 2051 | CA | PHE B | 390 | 62.928 | 8.778 | 55.382 | 1.00 | 26.53 | B |
| ATOM | 2052 | CB | PHE B | 390 | 61.606 | 9.405 | 55.833 | 1.00 | 24.52 | B |
| ATOM | 2053 | CG | PHE B | 390 | 60.846 | 10.069 | 54.728 | 1.00 | 23.83 | B |
| ATOM | 2054 | CD1 | PHE B | 390 | 60.614 | 9.408 | 53.530 | 1.00 | 24.31 | B |
| ATOM | 2055 | CD2 | PHE B | 390 | 60.357 | 11.358 | 54.883 | 1.00 | 24.82 | B |
| ATOM | 2056 | CE1 | PHE B | 390 | 59.906 | 10.021 | 52.496 | 1.00 | 24.43 | B |
| ATOM | 2057 | CE2 | PHE B | 390 | 59.643 | 11.985 | 53.851 | 1.00 | 26.18 | B |
| ATOM | 2058 | CZ | PHE B | 390 | 59.418 | 11.316 | 52.657 | 1.00 | 25.05 | B |
| ATOM | 2059 | C | PHE B | 390 | 63.885 | 9.852 | 54.853 | 1.00 | 28.00 | B |
| ATOM | 2060 | O | PHE B | 390 | 64.003 | 10.930 | 55.439 | 1.00 | 26.52 | B |
| ATOM | 2061 | N | GLU B | 391 | 64.591 | 9.532 | 53.761 | 1.00 | 31.44 | B |
| ATOM | 2062 | CA | GLU B | 391 | 65.552 | 10.459 | 53.112 | 1.00 | 34.19 | B |
| ATOM | 2063 | CB | GLU B | 391 | 66.958 | 10.374 | 53.753 | 1.00 | 35.50 | B |
| ATOM | 2064 | CG | GLU B | 391 | 67.082 | 9.731 | 55.143 | 1.00 | 38.57 | B |
| ATOM | 2065 | CD | GLU B | 391 | 66.652 | 10.654 | 56.306 | 1.00 | 40.82 | B |
| ATOM | 2066 | OE1 | GLU B | 391 | 66.842 | 11.896 | 56.199 | 1.00 | 38.26 | B |
| ATOM | 2067 | OE2 | GLU B | 391 | 66.138 | 10.116 | 57.327 | 1.00 | 41.64 | B |
| ATOM | 2068 | C | GLU B | 391 | 65.706 | 10.165 | 51.602 | 1.00 | 34.30 | B |
| ATOM | 2069 | O | GLU B | 391 | 65.415 | 9.060 | 51.141 | 1.00 | 32.53 | B |
| ATOM | 2070 | N | ASP B | 392 | 66.179 | 11.151 | 50.842 | 1.00 | 35.27 | B |
| ATOM | 2071 | CA | ASP B | 392 | 66.388 | 10.979 | 49.395 | 1.00 | 35.87 | B |
| ATOM | 2072 | CB | ASP B | 392 | 67.633 | 10.114 | 49.106 | 1.00 | 35.92 | B |
| ATOM | 2073 | CG | ASP B | 392 | 68.941 | 10.886 | 49.139 | 1.00 | 36.64 | B |
| ATOM | 2074 | OD1 | ASP B | 392 | 68.967 | 12.093 | 48.795 | 1.00 | 37.84 | B |
| ATOM | 2075 | OD2 | ASP B | 392 | 69.964 | 10.253 | 49.492 | 1.00 | 36.21 | B |
| ATOM | 2076 | C | ASP B | 392 | 65.215 | 10.300 | 48.673 | 1.00 | 36.11 | B |
| ATOM | 2077 | O | ASP B | 392 | 65.452 | 9.465 | 47.805 | 1.00 | 36.71 | B |
| ATOM | 2078 | N | ASN B | 393 | 63.968 | 10.618 | 49.018 | 1.00 | 35.71 | B |
| ATOM | 2079 | CA | ASN B | 393 | 62.805 | 9.999 | 48.347 | 1.00 | 35.51 | B |
| ATOM | 2080 | CB | ASN B | 393 | 62.861 | 10.281 | 46.847 | 1.00 | 34.88 | B |
| ATOM | 2081 | CG | ASN B | 393 | 62.762 | 11.755 | 46.544 | 1.00 | 37.09 | B |
| ATOM | 2082 | OD1 | ASN B | 393 | 61.672 | 12.328 | 46.584 | 1.00 | 36.85 | B |
| ATOM | 2083 | ND2 | ASN B | 393 | 63.903 | 12.388 | 46.255 | 1.00 | 37.57 | B |
| ATOM | 2084 | C | ASN B | 393 | 62.633 | 8.489 | 48.570 | 1.00 | 35.26 | B |
| ATOM | 2085 | O | ASN B | 393 | 61.925 | 7.805 | 47.817 | 1.00 | 34.05 | B |
| ATOM | 2086 | N | TYR B | 394 | 63.283 | 7.990 | 49.618 | 1.00 | 35.44 | B |
| ATOM | 2087 | CA | TYR B | 394 | 63.246 | 6.585 | 50.004 | 1.00 | 33.92 | B |
| ATOM | 2088 | CB | TYR B | 394 | 64.660 | 6.006 | 50.087 | 1.00 | 34.53 | B |
| ATOM | 2089 | CG | TYR B | 394 | 65.314 | 5.705 | 48.772 | 1.00 | 35.08 | B |
| ATOM | 2090 | CD1 | TYR B | 394 | 64.808 | 4.707 | 47.944 | 1.00 | 33.82 | B |
| ATOM | 2091 | CE1 | TYR B | 394 | 65.424 | 4.394 | 46.753 | 1.00 | 35.67 | B |
| ATOM | 2092 | CD2 | TYR B | 394 | 66.461 | 6.393 | 48.370 | 1.00 | 33.92 | B |
| ATOM | 2093 | CE2 | TYR B | 394 | 67.093 | 6.091 | 47.179 | 1.00 | 36.23 | B |
| ATOM | 2094 | CZ | TYR B | 394 | 66.571 | 5.090 | 46.369 | 1.00 | 37.49 | B |
| ATOM | 2095 | OH | TYR B | 394 | 67.184 | 4.785 | 45.167 | 1.00 | 39.46 | B |
| ATOM | 2096 | C | TYR B | 394 | 62.652 | 6.473 | 51.393 | 1.00 | 33.94 | B |
| ATOM | 2097 | O | TYR B | 394 | 62.973 | 7.268 | 52.281 | 1.00 | 32.72 | B |
| ATOM | 2098 | N | VAL B | 395 | 61.804 | 5.470 | 51.575 | 1.00 | 33.59 | B |
| ATOM | 2099 | CA | VAL B | 395 | 61.200 | 5.196 | 52.864 | 1.00 | 33.27 | B |
| ATOM | 2100 | CB | VAL B | 395 | 59.655 | 5.170 | 52.787 | 1.00 | 33.84 | B |
| ATOM | 2101 | CG1 | VAL B | 395 | 59.055 | 4.927 | 54.167 | 1.00 | 32.25 | B |
| ATOM | 2102 | CG2 | VAL B | 395 | 59.145 | 6.467 | 52.213 | 1.00 | 34.69 | B |
| ATOM | 2103 | C | VAL B | 395 | 61.708 | 3.803 | 53.182 | 1.00 | 33.68 | B |
| ATOM | 2104 | O | VAL B | 395 | 61.334 | 2.822 | 52.526 | 1.00 | 35.51 | B |
| ATOM | 2105 | N | ILE B | 396 | 62.592 | 3.701 | 54.163 | 1.00 | 33.52 | B |
| ATOM | 2106 | CA | ILE B | 396 | 63.113 | 2.386 | 54.508 | 1.00 | 32.85 | B |
| ATOM | 2107 | CB | ILE B | 396 | 64.666 | 2.403 | 54.579 | 1.00 | 33.18 | B |
| ATOM | 2108 | CG2 | ILE B | 396 | 65.207 | 0.998 | 54.438 | 1.00 | 32.95 | B |
| ATOM | 2109 | CG1 | ILE B | 396 | 65.238 | 3.231 | 53.434 | 1.00 | 32.57 | B |
| ATOM | 2110 | CD1 | ILE B | 396 | 66.704 | 3.519 | 53.594 | 1.00 | 34.98 | B |
| ATOM | 2111 | C | ILE B | 396 | 62.527 | 1.999 | 55.862 | 1.00 | 30.96 | B |
| ATOM | 2112 | O | ILE B | 396 | 62.550 | 2.787 | 56.805 | 1.00 | 29.47 | B |
| ATOM | 2113 | N | THR B | 397 | 61.977 | 0.792 | 55.943 | 1.00 | 29.81 | B |
| ATOM | 2114 | CA | THR B | 397 | 61.397 | 0.303 | 57.188 | 1.00 | 30.52 | B |
| ATOM | 2115 | CB | THR B | 397 | 59.883 | 0.014 | 57.087 | 1.00 | 28.68 | B |
| ATOM | 2116 | OG1 | THR B | 397 | 59.685 | −1.096 | 56.216 | 1.00 | 27.52 | B |
| ATOM | 2117 | CG2 | THR B | 397 | 59.127 | 1.206 | 56.561 | 1.00 | 28.88 | B |
| ATOM | 2118 | C | THR B | 397 | 62.052 | −1.012 | 57.574 | 1.00 | 32.66 | B |
| ATOM | 2119 | O | THR B | 397 | 62.495 | −1.768 | 56.708 | 1.00 | 32.93 | B |
| ATOM | 2120 | N | GLY B | 398 | 62.095 | −1.283 | 58.878 | 1.00 | 34.04 | B |
| ATOM | 2121 | CA | GLY B | 398 | 62.673 | −2.521 | 59.377 | 1.00 | 36.31 | B |
| ATOM | 2122 | C | GLY B | 398 | 61.790 | −3.205 | 60.409 | 1.00 | 38.57 | B |
| ATOM | 2123 | O | GLY B | 398 | 61.203 | −2.535 | 61.257 | 1.00 | 38.84 | B |
| ATOM | 2124 | N | ALA B | 399 | 61.692 | −4.532 | 60.347 | 1.00 | 40.48 | B |
| ATOM | 2125 | CA | ALA B | 399 | 60.870 | −5.274 | 61.304 | 1.00 | 43.09 | B |
| ATOM | 2126 | CB | ALA B | 399 | 59.540 | −5.687 | 60.660 | 1.00 | 43.54 | B |
| ATOM | 2127 | C | ALA B | 399 | 61.577 | −6.506 | 61.870 | 1.00 | 44.04 | B |
| ATOM | 2128 | O | ALA B | 399 | 62.688 | −6.838 | 61.461 | 1.00 | 43.75 | B |
| ATOM | 2129 | N | ASP B | 400 | 60.921 | −7.182 | 62.811 | 1.00 | 45.72 | B |
| ATOM | 2130 | CA | ASP B | 400 | 61.498 | −8.363 | 63.446 | 1.00 | 47.39 | B |
| ATOM | 2131 | CB | ASP B | 400 | 60.854 | −8.596 | 64.820 | 1.00 | 48.07 | B |
| ATOM | 2132 | CG | ASP B | 400 | 61.569 | −9.666 | 65.626 | 1.00 | 48.91 | B |
| ATOM | 2133 | OD1 | ASP B | 400 | 62.791 | −9.515 | 65.869 | 1.00 | 49.38 | B |
| ATOM | 2134 | OD2 | ASP B | 400 | 60.910 | −10.656 | 66.012 | 1.00 | 48.62 | B |
| ATOM | 2135 | C | ASP B | 400 | 61.347 | −9.610 | 62.579 | 1.00 | 47.48 | B |
| ATOM | 2136 | O | ASP B | 400 | 61.522 | −10.735 | 63.048 | 1.00 | 49.06 | B |
| ATOM | 2137 | N | ASP B | 401 | 61.014 | −9.403 | 61.313 | 1.00 | 46.02 | B |
| ATOM | 2138 | CA | ASP B | 401 | 60.851 | −10.501 | 60.378 | 1.00 | 43.80 | B |
| ATOM | 2139 | CB | ASP B | 401 | 59.688 | −10.192 | 59.436 | 1.00 | 45.11 | B |
| ATOM | 2140 | CG | ASP B | 401 | 59.962 | −8.996 | 58.541 | 1.00 | 46.35 | B |
| ATOM | 2141 | OD1 | ASP B | 401 | 60.797 | −8.130 | 58.909 | 1.00 | 46.25 | B |
| ATOM | 2142 | OD2 | ASP B | 401 | 59.325 | −8.919 | 57.465 | 1.00 | 47.47 | B |
| ATOM | 2143 | C | ASP B | 401 | 62.147 | −10.661 | 59.592 | 1.00 | 42.18 | B |
| ATOM | 2144 | O | ASP B | 401 | 62.153 | −11.209 | 58.498 | 1.00 | 40.84 | B |
| ATOM | 2145 | N | LYS B | 402 | 63.241 | −10.171 | 60.174 | 1.00 | 42.03 | B |
| ATOM | 2146 | CA | LYS B | 402 | 64.581 | −10.221 | 59.577 | 1.00 | 41.37 | B |
| ATOM | 2147 | CB | LYS B | 402 | 65.061 | −11.671 | 59.394 | 1.00 | 41.89 | B |
| ATOM | 2148 | CG | LYS B | 402 | 64.777 | −12.595 | 60.546 | 1.00 | 40.16 | B |
| ATOM | 2149 | CD | LYS B | 402 | 65.248 | −14.004 | 60.249 | 1.00 | 39.21 | B |
| ATOM | 2150 | CE | LYS B | 402 | 64.973 | −14.910 | 61.453 | 1.00 | 41.18 | B |
| ATOM | 2151 | NZ | LYS B | 402 | 65.506 | −16.306 | 61.313 | 1.00 | 41.31 | B |
| ATOM | 2152 | C | LYS B | 402 | 64.584 | −9.545 | 58.217 | 1.00 | 41.18 | B |
| ATOM | 2153 | O | LYS B | 402 | 65.387 | −9.880 | 57.350 | 1.00 | 41.03 | B |
| ATOM | 2154 | N | MSE B | 403 | 63.700 | −8.583 | 58.021 | 1.00 | 41.52 | B |
| ATOM | 2155 | CA | MSE B | 403 | 63.643 | −7.932 | 56.731 | 1.00 | 41.52 | B |
| ATOM | 2156 | CB | MSE B | 403 | 62.364 | −8.354 | 56.024 | 1.00 | 44.20 | B |
| ATOM | 2157 | CG | MSE B | 403 | 62.593 | −8.927 | 54.649 | 1.00 | 49.51 | B |
| ATOM | 2158 | CE | MSE B | 403 | 63.401 | −10.704 | 54.630 | 1.00 | 56.96 | B |
| ATOM | 2159 | CE | MSE B | 403 | 61.754 | −11.724 | 54.910 | 1.00 | 52.63 | B |
| ATOM | 2160 | C | MSE B | 403 | 63.728 | −6.408 | 56.765 | 1.00 | 39.82 | B |
| ATOM | 2161 | O | MSE B | 403 | 63.396 | −5.758 | 57.763 | 1.00 | 39.40 | B |
| ATOM | 2162 | N | ILE B | 404 | 64.163 | −5.861 | 55.638 | 1.00 | 37.19 | B |
| ATOM | 2163 | CA | ILE B | 404 | 64.305 | −4.432 | 55.441 | 1.00 | 34.55 | B |
| ATOM | 2164 | CB | ILE B | 404 | 65.790 | −4.049 | 55.290 | 1.00 | 33.79 | B |
| ATOM | 2165 | CG2 | ILE B | 404 | 65.904 | −2.619 | 54.750 | 1.00 | 32.01 | B |
| ATOM | 2166 | CG1 | ILE B | 404 | 66.516 | −4.256 | 56.628 | 1.00 | 32.98 | B |
| ATOM | 2167 | CD1 | ILE B | 404 | 68.003 | −3.868 | 56.634 | 1.00 | 32.54 | B |
| ATOM | 2168 | C | ILE B | 404 | 63.576 | −4.060 | 54.152 | 1.00 | 34.51 | B |
| ATOM | 2169 | O | ILE B | 404 | 63.994 | −4.455 | 53.061 | 1.00 | 33.17 | B |
| ATOM | 2170 | N | ARG B | 405 | 62.489 | −3.303 | 54.275 | 1.00 | 34.07 | B |
| ATOM | 2171 | CA | ARG B | 405 | 61.714 | −2.890 | 53.104 | 1.00 | 34.11 | B |
| ATOM | 2172 | CB | ARG B | 405 | 60.222 | −3.145 | 53.349 | 1.00 | 31.76 | B |
| ATOM | 2173 | CG | ARG B | 405 | 59.877 | −4.612 | 53.569 | 1.00 | 29.64 | B |
| ATOM | 2174 | CD | ARG B | 405 | 58.419 | −4.820 | 53.979 | 1.00 | 28.14 | B |
| ATOM | 2175 | NE | ARG B | 405 | 57.449 | −4.457 | 52.948 | 1.00 | 28.57 | B |
| ATOM | 2176 | CZ | ARG B | 405 | 57.252 | −5.129 | 51.813 | 1.00 | 28.90 | B |
| ATOM | 2177 | NH1 | ARG B | 405 | 57.964 | −6.215 | 51.537 | 1.00 | 25.04 | B |
| ATOM | 2178 | NH2 | ARG B | 405 | 56.311 | −4.728 | 50.960 | 1.00 | 29.02 | B |
| ATOM | 2179 | C | ARG B | 405 | 61.912 | −1.425 | 52.730 | 1.00 | 34.97 | B |
| ATOM | 2180 | O | ARG B | 405 | 61.823 | −0.545 | 53.580 | 1.00 | 37.29 | B |
| ATOM | 2181 | N | VAL B | 406 | 62.180 | −1.157 | 51.460 | 1.00 | 34.65 | B |
| ATOM | 2182 | CA | VAL B | 406 | 62.353 | 0.218 | 51.027 | 1.00 | 35.39 | B |
| ATOM | 2183 | CB | VAL B | 406 | 63.710 | 0.421 | 50.341 | 1.00 | 38.27 | B |
| ATOM | 2184 | CG1 | VAL B | 406 | 63.886 | −0.606 | 49.251 | 1.00 | 39.50 | B |
| ATOM | 2185 | CG2 | VAL B | 406 | 63.789 | 1.831 | 49.759 | 1.00 | 38.99 | B |
| ATOM | 2186 | C | VAL B | 406 | 61.257 | 0.576 | 50.041 | 1.00 | 34.13 | B |
| ATOM | 2187 | O | VAL B | 406 | 60.867 | −0.250 | 49.213 | 1.00 | 35.33 | B |
| ATOM | 2188 | N | TYR B | 407 | 60.763 | 1.806 | 50.127 | 1.00 | 32.42 | B |
| ATOM | 2189 | CA | TYR B | 407 | 59.694 | 2.263 | 49.236 | 1.00 | 30.87 | B |
| ATOM | 2190 | CB | TYR B | 407 | 58.409 | 2.537 | 50.032 | 1.00 | 30.46 | B |
| ATOM | 2191 | CG | TYR B | 407 | 57.953 | 1.435 | 50.967 | 1.00 | 29.77 | B |
| ATOM | 2192 | CD1 | TYR B | 407 | 58.682 | 1.115 | 52.121 | 1.00 | 27.81 | B |
| ATOM | 2193 | CE1 | TYR B | 407 | 58.269 | 0.081 | 52.962 | 1.00 | 27.20 | B |
| ATOM | 2194 | CD2 | TYR B | 407 | 56.795 | 0.695 | 50.682 | 1.00 | 29.18 | B |
| ATOM | 2195 | CE2 | TYR B | 407 | 56.376 | −0.335 | 51.511 | 1.00 | 27.89 | B |
| ATOM | 2196 | CZ | TYR B | 407 | 57.114 | −0.645 | 52.646 | 1.00 | 27.80 | B |
| ATOM | 2197 | OH | TYR B | 407 | 56.711 | −1.705 | 53.430 | 1.00 | 26.41 | B |
| ATOM | 2198 | C | TYR B | 407 | 60.058 | 3.552 | 48.507 | 1.00 | 29.93 | B |
| ATOM | 2199 | O | TYR B | 407 | 61.078 | 4.180 | 48.790 | 1.00 | 30.68 | B |
| ATOM | 2200 | N | ASP B | 408 | 59.189 | 3.947 | 47.583 | 1.00 | 29.96 | B |
| ATOM | 2201 | CA | ASP B | 408 | 59.360 | 5.181 | 46.825 | 1.00 | 30.52 | B |
| ATOM | 2202 | CB | ASP B | 408 | 59.226 | 4.920 | 45.325 | 1.00 | 32.17 | B |
| ATOM | 2203 | CG | ASP B | 408 | 59.430 | 6.178 | 44.504 | 1.00 | 35.42 | B |
| ATOM | 2204 | OD1 | ASP B | 408 | 60.450 | 6.868 | 44.721 | 1.00 | 37.74 | B |
| ATOM | 2205 | OD2 | ASP B | 408 | 58.579 | 6.483 | 43.644 | 1.00 | 37.68 | B |
| ATOM | 2206 | C | ASP B | 408 | 58.296 | 6.195 | 47.264 | 1.00 | 29.92 | B |
| ATOM | 2207 | O | ASP B | 408 | 57.095 | 5.995 | 47.024 | 1.00 | 29.81 | B |
| ATOM | 2208 | N | SER B | 409 | 58.743 | 7.284 | 47.895 | 1.00 | 28.80 | B |
| ATOM | 2209 | CA | SER B | 409 | 57.848 | 8.338 | 48.400 | 1.00 | 28.18 | B |
| ATOM | 2210 | CB | SER B | 409 | 58.581 | 9.202 | 49.442 | 1.00 | 25.85 | B |
| ATOM | 2211 | OG | SER B | 409 | 59.652 | 9.932 | 48.869 | 1.00 | 23.45 | B |
| ATOM | 2212 | C | SER B | 409 | 57.253 | 9.245 | 47.320 | 1.00 | 29.34 | B |
| ATOM | 2213 | O | SER B | 409 | 56.259 | 9.941 | 47.562 | 1.00 | 27.47 | B |
| ATOM | 2214 | N | ILE B | 410 | 57.861 | 9.224 | 46.133 | 1.00 | 31.65 | B |
| ATOM | 2215 | CA | ILE B | 410 | 57.421 | 10.037 | 44.994 | 1.00 | 34.03 | B |
| ATOM | 2216 | CB | ILE B | 410 | 58.558 | 10.242 | 43.972 | 1.00 | 35.56 | B |
| ATOM | 2217 | CG2 | ILE B | 410 | 58.032 | 11.052 | 42.783 | 1.00 | 34.67 | B |
| ATOM | 2218 | CG1 | ILE B | 410 | 59.747 | 10.947 | 44.616 | 1.00 | 34.10 | B |
| ATOM | 2219 | CD1 | ILE B | 410 | 60.988 | 10.910 | 43.752 | 1.00 | 33.88 | B |
| ATOM | 2220 | C | ILE B | 410 | 56.259 | 9.409 | 44.217 | 1.00 | 34.05 | B |
| ATOM | 2221 | O | ILE B | 410 | 55.330 | 10.101 | 43.818 | 1.00 | 33.24 | B |
| ATOM | 2222 | N | ASN B | 411 | 56.354 | 8.107 | 43.972 | 1.00 | 34.44 | B |
| ATOM | 2223 | CA | ASN B | 411 | 55.327 | 7.390 | 43.246 | 1.00 | 36.84 | B |
| ATOM | 2224 | CB | ASN B | 411 | 55.955 | 6.377 | 42.273 | 1.00 | 39.14 | B |
| ATOM | 2225 | CG | ASN B | 411 | 56.312 | 6.993 | 40.911 | 1.00 | 41.27 | B |
| ATOM | 2226 | OD1 | ASN B | 411 | 56.852 | 8.100 | 40.834 | 1.00 | 41.10 | B |
| ATOM | 2227 | ND2 | ASN B | 411 | 56.017 | 6.261 | 39.832 | 1.00 | 42.48 | B |
| ATOM | 2228 | C | ASN B | 411 | 54.428 | 6.663 | 44.238 | 1.00 | 37.14 | B |
| ATOM | 2229 | O | ASN B | 411 | 53.466 | 6.001 | 43.848 | 1.00 | 37.86 | B |
| ATOM | 2230 | N | LYS B | 412 | 54.748 | 6.776 | 45.522 | 1.00 | 36.65 | B |
| ATOM | 2231 | CA | LYS B | 412 | 53.944 | 6.121 | 46.546 | 1.00 | 35.90 | B |
| ATOM | 2232 | CB | LYS B | 412 | 52.537 | 6.742 | 46.595 | 1.00 | 35.46 | B |
| ATOM | 2233 | CG | LYS B | 412 | 52.373 | 7.994 | 47.470 | 1.00 | 34.91 | B |
| ATOM | 2234 | CD | LYS B | 412 | 53.251 | 9.163 | 47.023 | 1.00 | 33.80 | B |
| ATOM | 2235 | CE | LYS B | 412 | 52.905 | 10.407 | 47.809 | 1.00 | 32.17 | B |
| ATOM | 2236 | NZ | LYS B | 412 | 53.762 | 11.553 | 47.463 | 1.00 | 32.35 | B |
| ATOM | 2237 | C | LYS B | 412 | 53.812 | 4.629 | 46.256 | 1.00 | 35.57 | B |
| ATOM | 2238 | O | LYS B | 412 | 52.703 | 4.092 | 46.252 | 1.00 | 35.07 | B |
| ATOM | 2239 | N | LYS B | 413 | 54.932 | 3.959 | 46.007 | 1.00 | 35.95 | B |
| ATOM | 2240 | CA | LYS B | 413 | 54.901 | 2.523 | 45.721 | 1.00 | 36.16 | B |
| ATOM | 2241 | CB | LYS B | 413 | 54.977 | 2.254 | 44.205 | 1.00 | 36.93 | B |
| ATOM | 2242 | CG | LYS B | 413 | 53.730 | 2.627 | 43.387 | 1.00 | 40.07 | B |
| ATOM | 2243 | CD | LYS B | 413 | 53.913 | 2.308 | 41.900 | 1.00 | 41.03 | B |
| ATOM | 2244 | CE | LYS B | 413 | 52.629 | 2.529 | 41.105 | 1.00 | 42.27 | B |
| ATOM | 2245 | NZ | LYS B | 413 | 51.504 | 1.699 | 41.610 | 1.00 | 43.62 | B |
| ATOM | 2246 | C | LYS B | 413 | 56.047 | 1.798 | 46.402 | 1.00 | 35.81 | B |
| ATOM | 2247 | O | LYS B | 413 | 57.012 | 2.417 | 46.841 | 1.00 | 33.26 | B |
| ATOM | 2248 | N | PHE B | 414 | 55.904 | 0.481 | 46.503 | 1.00 | 38.03 | B |
| ATOM | 2249 | CA | PHE B | 414 | 56.909 | −0.402 | 47.090 | 1.00 | 39.76 | B |
| ATOM | 2250 | CB | PHE B | 414 | 56.267 | −1.749 | 47.454 | 1.00 | 40.36 | B |
| ATOM | 2251 | CG | PHE B | 414 | 57.257 | −2.806 | 47.872 | 1.00 | 42.43 | B |
| ATOM | 2252 | CD1 | PHE B | 414 | 57.994 | −2.667 | 49.046 | 1.00 | 43.87 | B |
| ATOM | 2253 | CD2 | PHE B | 414 | 57.499 | −3.914 | 47.058 | 1.00 | 41.81 | B |
| ATOM | 2254 | CE1 | PHE B | 414 | 58.965 | −3.614 | 49.398 | 1.00 | 43.95 | B |
| ATOM | 2255 | CE2 | PHE B | 414 | 58.466 | −4.863 | 47.401 | 1.00 | 41.35 | B |
| ATOM | 2256 | CZ | PHE B | 414 | 59.201 | −4.712 | 48.571 | 1.00 | 42.36 | B |
| ATOM | 2257 | C | PHE B | 414 | 57.970 | −0.598 | 46.003 | 1.00 | 40.17 | B |
| ATOM | 2258 | O | PHE B | 414 | 57.642 | −0.894 | 44.854 | 1.00 | 39.12 | B |
| ATOM | 2259 | N | LEU B | 415 | 59.236 | −0.425 | 46.361 | 1.00 | 41.37 | B |
| ATOM | 2260 | CA | LEU B | 415 | 60.305 | −0.558 | 45.386 | 1.00 | 42.80 | B |
| ATOM | 2261 | CB | LEU B | 415 | 61.350 | 0.539 | 45.608 | 1.00 | 44.70 | B |
| ATOM | 2262 | CG | LEU B | 415 | 62.211 | 0.891 | 44.391 | 1.00 | 46.65 | B |
| ATOM | 2263 | CD1 | LEU B | 415 | 61.335 | 1.453 | 43.273 | 1.00 | 46.10 | B |
| ATOM | 2264 | CD2 | LEU B | 415 | 63.277 | 1.905 | 44.794 | 1.00 | 47.87 | B |
| ATOM | 2265 | C | LEU B | 415 | 60.950 | −1.941 | 45.464 | 1.00 | 43.04 | B |
| ATOM | 2266 | O | LEU B | 415 | 60.930 | −2.699 | 44.503 | 1.00 | 42.67 | B |
| ATOM | 2267 | N | LEU B | 416 | 61.531 | −2.288 | 46.600 | 1.00 | 43.99 | B |
| ATOM | 2268 | CA | LEU B | 416 | 62.140 | −3.607 | 46.711 | 1.00 | 44.67 | B |
| ATOM | 2269 | CB | LEU B | 416 | 63.471 | −3.667 | 45.948 | 1.00 | 43.56 | B |
| ATOM | 2270 | CG | LEU B | 416 | 64.709 | −3.198 | 46.703 | 1.00 | 42.12 | B |
| ATOM | 2271 | CD1 | LEU B | 416 | 65.621 | −4.380 | 46.873 | 1.00 | 45.95 | B |
| ATOM | 2272 | CD2 | LEU B | 416 | 65.421 | −2.101 | 45.951 | 1.00 | 41.53 | B |
| ATOM | 2273 | C | LEU B | 416 | 62.363 | −3.960 | 48.165 | 1.00 | 44.94 | B |
| ATOM | 2274 | O | LEU B | 416 | 62.183 | −3.123 | 49.060 | 1.00 | 45.50 | B |
| ATOM | 2275 | N | GLN B | 417 | 62.747 | −5.212 | 48.393 | 1.00 | 45.21 | B |
| ATOM | 2276 | CA | GLN B | 417 | 62.999 | −5.712 | 49.744 | 1.00 | 45.08 | B |
| ATOM | 2277 | CB | GLN B | 417 | 61.984 | −6.808 | 50.120 | 1.00 | 43.81 | B |
| ATOM | 2278 | CG | GLN B | 417 | 62.106 | −7.321 | 51.555 | 1.00 | 40.41 | B |
| ATOM | 2279 | CD | GLN B | 417 | 61.077 | −8.383 | 51.890 | 1.00 | 39.74 | B |
| ATOM | 2280 | OE1 | GLN B | 417 | 61.135 | −9.507 | 51.390 | 1.00 | 41.92 | B |
| ATOM | 2281 | NE2 | GLN B | 417 | 60.129 | −8.033 | 52.741 | 1.00 | 38.39 | B |
| ATOM | 2282 | C | GLN B | 417 | 64.412 | −6.279 | 49.844 | 1.00 | 44.86 | B |
| ATOM | 2283 | O | GLN B | 417 | 64.906 | −6.895 | 48.897 | 1.00 | 43.18 | B |
| ATOM | 2284 | N | LEU B | 418 | 65.053 | −6.053 | 50.991 | 1.00 | 45.20 | B |
| ATOM | 2285 | CA | LEU B | 418 | 66.408 | −6.536 | 51.246 | 1.00 | 44.82 | B |
| ATOM | 2286 | CB | LEU B | 418 | 67.329 | −5.373 | 51.654 | 1.00 | 42.07 | B |
| ATOM | 2287 | CG | LEU B | 418 | 67.665 | −4.358 | 50.550 | 1.00 | 40.04 | B |
| ATOM | 2288 | CD1 | LEU B | 418 | 67.984 | −2.989 | 51.123 | 1.00 | 38.25 | B |
| ATOM | 2289 | CD2 | LEU B | 418 | 68.804 | −4.885 | 49.746 | 1.00 | 39.20 | B |
| ATOM | 2290 | C | LEU B | 418 | 66.337 | −7.584 | 52.357 | 1.00 | 46.53 | B |
| ATOM | 2291 | O | LEU B | 418 | 65.728 | −7.355 | 53.411 | 1.00 | 46.91 | B |
| ATOM | 2292 | N | SER B | 419 | 66.929 | −8.747 | 52.086 | 1.00 | 47.50 | B |
| ATOM | 2293 | CA | SER B | 419 | 66.966 | −9.854 | 53.037 | 1.00 | 47.26 | B |
| ATOM | 2294 | CB | SER B | 419 | 66.219 | −11.066 | 52.489 | 1.00 | 47.58 | B |
| ATOM | 2295 | OG | SER B | 419 | 66.861 | −11.552 | 51.330 | 1.00 | 47.48 | B |
| ATOM | 2296 | C | SER B | 419 | 68.423 | −10.219 | 53.282 | 1.00 | 47.06 | B |
| ATOM | 2297 | O | SER B | 419 | 69.267 | −10.064 | 52.396 | 1.00 | 46.04 | B |
| ATOM | 2298 | N | GLY B | 420 | 68.700 | −10.697 | 54.493 | 1.00 | 47.22 | B |
| ATOM | 2299 | CA | GLY B | 420 | 70.047 | −11.059 | 54.886 | 1.00 | 46.53 | B |
| ATOM | 2300 | C | GLY B | 420 | 70.121 | −11.386 | 56.366 | 1.00 | 47.16 | B |
| ATOM | 2301 | O | GLY B | 420 | 70.335 | −12.546 | 56.715 | 1.00 | 48.39 | B |
| ATOM | 2302 | N | HIS B | 421 | 69.927 | −10.390 | 57.237 | 1.00 | 46.12 | B |
| ATOM | 2303 | CA | HIS B | 421 | 69.999 | −10.611 | 58.697 | 1.00 | 44.26 | B |
| ATOM | 2304 | CB | HIS B | 421 | 69.340 | −9.451 | 59.469 | 1.00 | 42.53 | B |
| ATOM | 2305 | CG | HIS B | 421 | 70.144 | −8.187 | 59.459 | 1.00 | 41.48 | B |
| ATOM | 2306 | CD2 | HIS B | 421 | 70.083 | −7.102 | 58.653 | 1.00 | 41.32 | B |
| ATOM | 2307 | ND1 | HIS B | 421 | 71.231 | −7.991 | 60.282 | 1.00 | 41.41 | B |
| ATOM | 2308 | CE1 | HIS B | 421 | 71.811 | −6.845 | 59.977 | 1.00 | 41.09 | B |
| ATOM | 2309 | NE2 | HIS B | 421 | 71.135 | −6.286 | 58.990 | 1.00 | 40.64 | B |
| ATOM | 2310 | C | HIS B | 421 | 69.374 | −11.937 | 59.142 | 1.00 | 44.07 | B |
| ATOM | 2311 | O | HIS B | 421 | 68.254 | −12.277 | 58.746 | 1.00 | 43.02 | B |
| ATOM | 2312 | N | ASP B | 422 | 70.103 | −12.687 | 59.964 | 1.00 | 43.55 | B |
| ATOM | 2313 | CA | ASP B | 422 | 69.599 | −13.967 | 60.447 | 1.00 | 43.78 | B |
| ATOM | 2314 | CB | ASP B | 422 | 70.752 | −14.881 | 60.881 | 1.00 | 46.42 | B |
| ATOM | 2315 | CG | ASP B | 422 | 71.843 | −15.013 | 59.811 | 1.00 | 48.64 | B |
| ATOM | 2316 | OD1 | ASP B | 422 | 71.518 | −15.368 | 58.649 | 1.00 | 47.48 | B |
| ATOM | 2317 | OD2 | ASP B | 422 | 73.030 | −14.762 | 60.145 | 1.00 | 50.00 | B |
| ATOM | 2318 | C | ASP B | 422 | 68.634 | −13.762 | 61.609 | 1.00 | 42.50 | B |
| ATOM | 2319 | O | ASP B | 422 | 67.970 | −14.701 | 62.048 | 1.00 | 41.14 | B |
| ATOM | 2320 | N | GLY B | 423 | 68.561 | −12.527 | 62.099 | 1.00 | 42.77 | B |
| ATOM | 2321 | CA | GLY B | 423 | 67.659 | −12.192 | 63.195 | 1.00 | 42.60 | B |
| ATOM | 2322 | C | GLY B | 423 | 66.843 | −10.950 | 62.865 | 1.00 | 41.88 | B |
| ATOM | 2323 | O | GLY B | 423 | 67.107 | −10.300 | 61.855 | 1.00 | 42.25 | B |
| ATOM | 2324 | N | GLY B | 424 | 65.854 | −10.615 | 63.694 | 1.00 | 41.87 | B |
| ATOM | 2325 | CA | GLY B | 424 | 65.036 | −9.432 | 63.433 | 1.00 | 41.62 | B |
| ATOM | 2326 | C | GLY B | 424 | 65.863 | −8.158 | 63.492 | 1.00 | 41.35 | B |
| ATOM | 2327 | O | GLY B | 424 | 66.673 | −7.994 | 64.389 | 1.00 | 41.29 | B |
| ATOM | 2328 | N | VAL B | 425 | 65.682 | −7.254 | 62.541 | 1.00 | 41.10 | B |
| ATOM | 2329 | CA | VAL B | 425 | 66.452 | −6.015 | 62.557 | 1.00 | 42.45 | B |
| ATOM | 2330 | CB | VAL B | 425 | 66.622 | −5.464 | 61.137 | 1.00 | 44.73 | B |
| ATOM | 2331 | CG1 | VAL B | 425 | 65.322 | −5.641 | 60.365 | 1.00 | 45.42 | B |
| ATOM | 2332 | CG2 | VAL B | 425 | 67.028 | −3.987 | 61.187 | 1.00 | 45.68 | B |
| ATOM | 2333 | C | VAL B | 425 | 65.796 | −4.946 | 63.433 | 1.00 | 41.72 | B |
| ATOM | 2334 | O | VAL B | 425 | 64.600 | −4.702 | 63.318 | 1.00 | 42.42 | B |
| ATOM | 2335 | N | TRP B | 426 | 66.575 | −4.305 | 64.301 | 1.00 | 40.55 | B |
| ATOM | 2336 | CA | TRP B | 426 | 66.016 | −3.284 | 65.177 | 1.00 | 39.71 | B |
| ATOM | 2337 | CB | TRP B | 426 | 66.069 | −3.755 | 66.632 | 1.00 | 36.69 | B |
| ATOM | 2338 | CG | TRP B | 426 | 65.117 | −4.874 | 66.920 | 1.00 | 34.11 | B |
| ATOM | 2339 | CD2 | TRP B | 426 | 63.831 | −4.774 | 67.558 | 1.00 | 32.48 | B |
| ATOM | 2340 | CE2 | TRP B | 426 | 63.275 | −6.072 | 67.578 | 1.00 | 30.88 | B |
| ATOM | 2341 | CE3 | TRP B | 426 | 63.098 | −3.715 | 68.110 | 1.00 | 30.22 | B |
| ATOM | 2342 | CD1 | TRP B | 426 | 65.278 | −6.185 | 66.594 | 1.00 | 33.27 | B |
| ATOM | 2343 | NE1 | TRP B | 426 | 64.179 | −6.910 | 66.984 | 1.00 | 32.30 | B |
| ATOM | 2344 | CZ2 | TRP B | 426 | 62.017 | −6.344 | 68.129 | 1.00 | 31.05 | B |
| ATOM | 2345 | CZ3 | TRP B | 426 | 61.845 | −3.984 | 68.661 | 1.00 | 30.71 | B |
| ATOM | 2346 | CH2 | TRP B | 426 | 61.319 | −5.289 | 68.666 | 1.00 | 30.24 | B |
| ATOM | 2347 | C | TRP B | 426 | 66.631 | −1.889 | 65.066 | 1.00 | 40.31 | B |
| ATOM | 2348 | O | TRP B | 426 | 66.089 | −0.930 | 65.607 | 1.00 | 40.76 | B |
| ATOM | 2349 | N | ALA B | 427 | 67.759 | −1.774 | 64.379 | 1.00 | 41.09 | B |
| ATOM | 2350 | CA | ALA B | 427 | 68.400 | −0.482 | 64.198 | 1.00 | 42.24 | B |
| ATOM | 2351 | CB | ALA B | 427 | 69.783 | −0.481 | 64.816 | 1.00 | 42.58 | B |
| ATOM | 2352 | C | ALA B | 427 | 68.488 | −0.284 | 62.695 | 1.00 | 43.39 | B |
| ATOM | 2353 | O | ALA B | 427 | 68.658 | −1.251 | 61.952 | 1.00 | 44.01 | B |
| ATOM | 2354 | N | LEU B | 428 | 68.375 | 0.963 | 62.248 | 1.00 | 44.59 | B |
| ATOM | 2355 | CA | LEU B | 428 | 68.401 | 1.261 | 60.821 | 1.00 | 44.89 | B |
| ATOM | 2356 | CB | LEU B | 428 | 67.015 | 0.996 | 60.230 | 1.00 | 45.56 | B |
| ATOM | 2357 | CG | LEU B | 428 | 66.894 | 0.895 | 58.717 | 1.00 | 46.48 | B |
| ATOM | 2358 | CD1 | LEU B | 428 | 67.730 | −0.281 | 58.221 | 1.00 | 47.28 | B |
| ATOM | 2359 | CD2 | LEU B | 428 | 65.434 | 0.710 | 58.349 | 1.00 | 46.47 | B |
| ATOM | 2360 | C | LEU B | 428 | 68.761 | 2.714 | 60.608 | 1.00 | 44.99 | B |
| ATOM | 2361 | O | LEU B | 428 | 68.132 | 3.596 | 61.178 | 1.00 | 46.41 | B |
| ATOM | 2362 | N | LYS B | 429 | 69.766 | 2.969 | 59.785 | 1.00 | 44.76 | B |
| ATOM | 2363 | CA | LYS B | 429 | 70.189 | 4.341 | 59.519 | 1.00 | 44.20 | B |
| ATOM | 2364 | CB | LYS B | 429 | 71.478 | 4.631 | 60.288 | 1.00 | 43.86 | B |
| ATOM | 2365 | CG | LYS B | 429 | 72.022 | 6.021 | 60.108 | 1.00 | 43.02 | B |
| ATOM | 2366 | CD | LYS B | 429 | 71.151 | 7.060 | 60.779 | 1.00 | 40.87 | B |
| ATOM | 2367 | CE | LYS B | 429 | 71.741 | 8.447 | 60.560 | 1.00 | 38.32 | B |
| ATOM | 2368 | NZ | LYS B | 429 | 71.164 | 9.464 | 61.464 | 1.00 | 34.95 | B |
| ATOM | 2369 | C | LYS B | 429 | 70.406 | 4.457 | 58.021 | 1.00 | 44.44 | B |
| ATOM | 2370 | O | LYS B | 429 | 70.534 | 3.445 | 57.344 | 1.00 | 44.65 | B |
| ATOM | 2371 | N | TYR B | 430 | 70.430 | 5.677 | 57.496 | 1.00 | 45.98 | B |
| ATOM | 2372 | CA | TYR B | 430 | 70.617 | 5.884 | 56.052 | 1.00 | 46.60 | B |
| ATOM | 2373 | CB | TYR B | 430 | 69.403 | 6.616 | 55.458 | 1.00 | 43.95 | B |
| ATOM | 2374 | CG | TYR B | 430 | 69.496 | 6.911 | 53.973 | 1.00 | 41.46 | B |
| ATOM | 2375 | CD1 | TYR B | 430 | 69.092 | 5.976 | 53.027 | 1.00 | 40.19 | B |
| ATOM | 2376 | CE1 | TYR B | 430 | 69.153 | 6.255 | 51.664 | 1.00 | 38.85 | B |
| ATOM | 2377 | CD2 | TYR B | 430 | 69.968 | 8.137 | 53.520 | 1.00 | 40.10 | B |
| ATOM | 2378 | CE2 | TYR B | 430 | 70.034 | 8.430 | 52.164 | 1.00 | 39.82 | B |
| ATOM | 2379 | CZ | TYR B | 430 | 69.625 | 7.486 | 51.232 | 1.00 | 39.47 | B |
| ATOM | 2380 | OH | TYR B | 430 | 69.691 | 7.784 | 49.874 | 1.00 | 37.41 | B |
| ATOM | 2381 | C | TYR B | 430 | 71.872 | 6.696 | 55.767 | 1.00 | 47.43 | B |
| ATOM | 2382 | O | TYR B | 430 | 72.320 | 7.476 | 56.603 | 1.00 | 48.99 | B |
| ATOM | 2383 | N | ALA B | 431 | 72.439 | 6.516 | 54.582 | 1.00 | 48.65 | B |
| ATOM | 2384 | CA | ALA B | 431 | 73.634 | 7.263 | 54.196 | 1.00 | 48.95 | B |
| ATOM | 2385 | CB | ALA B | 431 | 74.882 | 6.412 | 54.420 | 1.00 | 49.04 | B |
| ATOM | 2386 | C | ALA B | 431 | 73.533 | 7.683 | 52.732 | 1.00 | 49.00 | B |
| ATOM | 2387 | O | ALA B | 431 | 73.298 | 6.860 | 51.842 | 1.00 | 48.35 | B |
| ATOM | 2388 | N | HIS B | 432 | 73.729 | 8.967 | 52.482 | 1.00 | 49.81 | B |
| ATOM | 2389 | CA | HIS B | 432 | 73.627 | 9.485 | 51.126 | 1.00 | 52.51 | B |
| ATOM | 2390 | CB | HIS B | 432 | 74.421 | 10.787 | 50.978 | 1.00 | 55.86 | B |
| ATOM | 2391 | CG | HIS B | 432 | 74.148 | 11.508 | 49.695 | 1.00 | 60.52 | B |
| ATOM | 2392 | CD2 | HIS B | 432 | 74.984 | 12.012 | 48.757 | 1.00 | 62.00 | B |
| ATOM | 2393 | ND1 | HIS B | 432 | 72.869 | 11.750 | 49.238 | 1.00 | 62.73 | B |
| ATOM | 2394 | CE1 | HIS B | 432 | 72.930 | 12.368 | 48.072 | 1.00 | 63.55 | B |
| ATOM | 2395 | NE2 | HIS B | 432 | 74.202 | 12.539 | 47.757 | 1.00 | 63.41 | B |
| ATOM | 2396 | C | HIS B | 432 | 74.084 | 8.497 | 50.059 | 1.00 | 51.80 | B |
| ATOM | 2397 | O | HIS B | 432 | 75.031 | 7.737 | 50.266 | 1.00 | 53.10 | B |
| ATOM | 2398 | N | GLY B | 433 | 73.391 | 8.508 | 48.923 | 1.00 | 50.58 | B |
| ATOM | 2399 | CA | GLY B | 433 | 73.741 | 7.633 | 47.819 | 1.00 | 47.93 | B |
| ATOM | 2400 | C | GLY B | 433 | 72.921 | 6.368 | 47.714 | 1.00 | 47.35 | B |
| ATOM | 2401 | O | GLY B | 433 | 73.272 | 5.462 | 46.963 | 1.00 | 47.94 | B |
| ATOM | 2402 | N | GLY B | 434 | 71.823 | 6.293 | 48.452 | 1.00 | 46.94 | B |
| ATOM | 2403 | CA | GLY B | 434 | 71.016 | 5.090 | 48.392 | 1.00 | 45.51 | B |
| ATOM | 2404 | C | GLY B | 434 | 71.722 | 3.980 | 49.147 | 1.00 | 44.93 | B |
| ATOM | 2405 | O | GLY B | 434 | 71.599 | 2.804 | 48.824 | 1.00 | 45.17 | B |
| ATOM | 2406 | N | ILE B | 435 | 72.501 | 4.353 | 50.151 | 1.00 | 44.36 | B |
| ATOM | 2407 | CA | ILE B | 435 | 73.183 | 3.352 | 50.943 | 1.00 | 43.99 | B |
| ATOM | 2408 | CB | ILE B | 435 | 74.701 | 3.520 | 50.874 | 1.00 | 43.64 | B |
| ATOM | 2409 | CG2 | ILE B | 435 | 75.375 | 2.722 | 51.989 | 1.00 | 43.26 | B |
| ATOM | 2410 | CG1 | ILE B | 435 | 75.196 | 3.022 | 49.528 | 1.00 | 43.31 | B |
| ATOM | 2411 | CD1 | ILE B | 435 | 76.656 | 3.223 | 49.321 | 1.00 | 45.00 | B |
| ATOM | 2412 | C | ILE B | 435 | 72.724 | 3.435 | 52.395 | 1.00 | 44.68 | B |
| ATOM | 2413 | O | ILE B | 435 | 72.570 | 4.518 | 52.956 | 1.00 | 42.73 | B |
| ATOM | 2414 | N | LEU B | 436 | 72.486 | 2.283 | 53.000 | 1.00 | 44.69 | B |
| ATOM | 2415 | CA | LEU B | 436 | 72.051 | 2.273 | 54.376 | 1.00 | 46.18 | B |
| ATOM | 2416 | CB | LEU B | 436 | 70.545 | 2.006 | 54.468 | 1.00 | 46.74 | B |
| ATOM | 2417 | CG | LEU B | 436 | 70.020 | 0.623 | 54.084 | 1.00 | 46.70 | B |
| ATOM | 2418 | CD1 | LEU B | 436 | 68.660 | 0.398 | 54.708 | 1.00 | 47.92 | B |
| ATOM | 2419 | CD2 | LEU B | 436 | 69.953 | 0.506 | 52.574 | 1.00 | 48.12 | B |
| ATOM | 2420 | C | LEU B | 436 | 72.816 | 1.224 | 55.176 | 1.00 | 47.09 | B |
| ATOM | 2421 | O | LEU B | 436 | 73.360 | 0.268 | 54.619 | 1.00 | 46.99 | B |
| ATOM | 2422 | N | VAL B | 437 | 72.862 | 1.418 | 56.488 | 1.00 | 46.98 | B |
| ATOM | 2423 | CA | VAL B | 437 | 73.552 | 0.497 | 57.376 | 1.00 | 48.04 | B |
| ATOM | 2424 | CB | VAL B | 437 | 74.617 | 1.244 | 58.228 | 1.00 | 49.43 | B |
| ATOM | 2425 | CG1 | VAL B | 437 | 75.517 | 0.251 | 58.948 | 1.00 | 48.59 | B |
| ATOM | 2426 | CG2 | VAL B | 437 | 75.443 | 2.180 | 57.333 | 1.00 | 49.25 | B |
| ATOM | 2427 | C | VAL B | 437 | 72.481 | −0.062 | 58.299 | 1.00 | 46.89 | B |
| ATOM | 2428 | O | VAL B | 437 | 71.430 | 0.551 | 58.449 | 1.00 | 47.34 | B |
| ATOM | 2429 | N | SER B | 438 | 72.722 | −1.222 | 58.902 | 1.00 | 45.63 | B |
| ATOM | 2430 | CA | SER B | 438 | 71.741 | −1.788 | 59.820 | 1.00 | 44.89 | B |
| ATOM | 2431 | CB | SER B | 438 | 70.729 | −2.644 | 59.072 | 1.00 | 44.08 | B |
| ATOM | 2432 | OG | SER B | 438 | 71.355 | −3.758 | 58.467 | 1.00 | 44.83 | B |
| ATOM | 2433 | C | SER B | 438 | 72.377 | −2.622 | 60.914 | 1.00 | 44.82 | B |
| ATOM | 2434 | O | SER B | 438 | 73.561 | −2.949 | 60.857 | 1.00 | 44.21 | B |
| ATOM | 2435 | N | GLY B | 439 | 71.568 | −2.945 | 61.916 | 1.00 | 45.02 | B |
| ATOM | 2436 | CA | GLY B | 439 | 72.020 | ‘3.751 | 63.027 | 1.00 | 45.90 | B |
| ATOM | 2437 | C | GLY B | 439 | 70.855 | −4.596 | 63.498 | 1.00 | 46.75 | B |
| ATOM | 2438 | O | GLY B | 439 | 69.809 | −4.054 | 63.843 | 1.00 | 45.99 | B |
| ATOM | 2439 | N | SER B | 440 | 71.006 | −5.915 | 63.496 | 1.00 | 49.24 | B |
| ATOM | 2440 | CA | SER B | 440 | 69.919 | −6.773 | 63.957 | 1.00 | 53.65 | B |
| ATOM | 2441 | CB | SER B | 440 | 69.429 | −7.707 | 62.842 | 1.00 | 54.51 | B |
| ATOM | 2442 | OG | SER B | 440 | 70.342 | −8.766 | 62.610 | 1.00 | 56.40 | B |
| ATOM | 2443 | C | SER B | 440 | 70.368 | −7.612 | 65.146 | 1.00 | 54.82 | B |
| ATOM | 2444 | O | SER B | 440 | 71.474 | −7.447 | 65.660 | 1.00 | 54.64 | B |
| ATOM | 2445 | N | T5R B | 441 | 69.490 | −8.509 | 65.576 | 1.00 | 57.06 | B |
| ATOM | 2446 | CA | THR B | 441 | 69.769 | −9.392 | 66.695 | 1.00 | 59.13 | B |
| ATOM | 2447 | CB | THR B | 441 | 68.498 | −10.086 | 67.165 | 1.00 | 57.43 | B |
| ATOM | 2448 | OG1 | THR B | 441 | 67.489 | −9.101 | 67.397 | 1.00 | 55.10 | B |
| ATOM | 2449 | CG2 | THR B | 441 | 68.761 | −10.835 | 68.455 | 1.00 | 59.62 | B |
| ATOM | 2450 | C | T5R B | 441 | 70.778 | −10.442 | 66.262 | 1.00 | 61.73 | B |
| ATOM | 2451 | O | THR B | 441 | 71.212 | −11.280 | 67.048 | 1.00 | 62.25 | B |
| ATOM | 2452 | N | ASP B | 442 | 71.142 | −10.379 | 64.989 | 1.00 | 64.11 | B |
| ATOM | 2453 | CA | ASP B | 442 | 72.098 | −11.292 | 64.403 | 1.00 | 65.34 | B |
| ATOM | 2454 | CB | ASP B | 442 | 72.028 | −11.183 | 62.877 | 1.00 | 69.79 | B |
| ATOM | 2455 | CG | ASP B | 442 | 72.888 | −12.220 | 62.171 | 1.00 | 76.04 | B |
| ATOM | 2456 | OD1 | ASP B | 442 | 72.837 | −13.413 | 62.567 | 1.00 | 78.67 | B |
| ATOM | 2457 | OD2 | ASP B | 442 | 73.607 | −11.846 | 61.208 | 1.00 | 78.25 | B |
| ATOM | 2458 | C | ASP B | 442 | 73.490 | −10.929 | 64.903 | 1.00 | 64.29 | B |
| ATOM | 2459 | O | ASP B | 442 | 74.469 | −11.621 | 64.626 | 1.00 | 65.24 | B |
| ATOM | 2460 | N | ARG B | 443 | 73.561 | −9.843 | 65.662 | 1.00 | 62.52 | B |
| ATOM | 2461 | CA | ARG B | 443 | 74.817 | −9.346 | 66.214 | 1.00 | 60.60 | B |
| ATOM | 2462 | CB | ARG B | 443 | 75.495 | −10.412 | 67.088 | 1.00 | 61.78 | B |
| ATOM | 2463 | CG | ARG B | 443 | 74.629 | −10.891 | 68.231 | 1.00 | 64.13 | B |
| ATOM | 2464 | CD | ARG B | 443 | 75.329 | −11.918 | 69.100 | 1.00 | 65.45 | B |
| ATOM | 2465 | NE | ARG B | 443 | 75.805 | −13.045 | 68.312 | 1.00 | 68.14 | B |
| ATOM | 2466 | CZ | ARG B | 443 | 76.062 | −14.252 | 68.804 | 1.00 | 69.90 | B |
| ATOM | 2467 | NH1 | ARG B | 443 | 75.885 | −14.495 | 70.096 | 1.00 | 69.79 | B |
| ATOM | 2468 | NH2 | ARG B | 443 | 76.493 | −15.219 | 67.998 | 1.00 | 70.62 | B |
| ATOM | 2469 | C | ARG B | 443 | 75.754 | −8.904 | 65.100 | 1.00 | 58.12 | B |
| ATOM | 2470 | O | ARG B | 443 | 76.972 | −8.886 | 65.266 | 1.00 | 58.34 | B |
| ATOM | 2471 | N | THR B | 444 | 75.183 | −8.534 | 63.960 | 1.00 | 55.54 | B |
| ATOM | 2472 | CA | THR B | 444 | 76.005 | −8.092 | 62.841 | 1.00 | 53.90 | B |
| ATOM | 2473 | CB | THR B | 444 | 75.945 | −9.106 | 61.671 | 1.00 | 54.03 | B |
| ATOM | 2474 | OG1 | THR B | 444 | 74.590 | −9.262 | 61.232 | 1.00 | 53.30 | B |
| ATOM | 2475 | CG2 | THR B | 444 | 76.482 | −10.464 | 62.113 | 1.00 | 53.83 | B |
| ATOM | 2476 | C | THR B | 444 | 75.567 | −6.727 | 62.338 | 1.00 | 51.81 | B |
| ATOM | 2477 | O | THR B | 444 | 74.401 | −6.377 | 62.409 | 1.00 | 52.22 | B |
| ATOM | 2478 | N | VAL B | 445 | 76.510 | −5.945 | 61.846 | 1.00 | 49.88 | B |
| ATOM | 2479 | CA | VAL B | 445 | 76.169 | −4.641 | 61.330 | 1.00 | 49.27 | B |
| ATOM | 2480 | CB | VAL B | 445 | 77.158 | −3.569 | 61.825 | 1.00 | 48.34 | B |
| ATOM | 2481 | CG1 | VAL B | 445 | 76.756 | −2.196 | 61.303 | 1.00 | 47.11 | B |
| ATOM | 2482 | CG2 | VAL B | 445 | 77.192 | −3.566 | 63.349 | 1.00 | 48.46 | B |
| ATOM | 2483 | C | VAL B | 445 | 76.233 | −4.745 | 59.813 | 1.00 | 50.36 | B |
| ATOM | 2484 | O | VAL B | 445 | 77.308 | −4.656 | 59.209 | 1.00 | 50.35 | B |
| ATOM | 2485 | N | ARG B | 446 | 75.080 | −4.959 | 59.194 | 1.00 | 50.86 | B |
| ATOM | 2486 | CA | ARG B | 446 | 75.039 | −5.081 | 57.749 | 1.00 | 51.42 | B |
| ATOM | 2487 | CB | ARG B | 446 | 74.024 | −6.152 | 57.329 | 1.00 | 52.81 | B |
| ATOM | 2488 | CG | ARG B | 446 | 74.384 | −7.595 | 57.725 | 1.00 | 53.32 | B |
| ATOM | 2489 | CD | ARG B | 446 | 73.563 | −8.565 | 56.893 | 1.00 | 54.70 | B |
| ATOM | 2490 | NE | ARG B | 446 | 73.801 | −9.970 | 57.206 | 1.00 | 55.46 | B |
| ATOM | 2491 | CZ | ARG B | 446 | 73.462 | −10.545 | 58.353 | 1.00 | 57.14 | B |
| ATOM | 2492 | NH1 | ARG B | 446 | 72.878 | −9.834 | 59.303 | 1.00 | 59.85 | B |
| ATOM | 2493 | NH2 | ARG B | 446 | 73.677 | −11.839 | 58.548 | 1.00 | 57.51 | B |
| ATOM | 2494 | C | ARG B | 446 | 74.712 | −3.759 | 57.058 | 1.00 | 50.94 | B |
| ATOM | 2495 | O | ARG B | 446 | 73.764 | −3.066 | 57.427 | 1.00 | 51.11 | B |
| ATOM | 2496 | N | VAL B | 447 | 75.525 | −3.414 | 56.063 | 1.00 | 49.98 | B |
| ATOM | 2497 | CA | VAL B | 447 | 75.338 | −2.198 | 55.280 | 1.00 | 49.12 | B |
| ATOM | 2498 | CB | VAL B | 447 | 76.661 | −1.360 | 55.196 | 1.00 | 49.77 | B |
| ATOM | 2499 | CG1 | VAL B | 447 | 77.858 | −2.268 | 55.110 | 1.00 | 48.80 | B |
| ATOM | 2500 | CG2 | VAL B | 447 | 76.631 | −0.433 | 53.984 | 1.00 | 50.29 | B |
| ATOM | 2501 | C | VAL B | 447 | 74.876 | −2.650 | 53.893 | 1.00 | 47.59 | B |
| ATOM | 2502 | O | VAL B | 447 | 75.433 | −3.584 | 53.334 | 1.00 | 46.28 | B |
| ATOM | 2503 | N | TRP B | 448 | 73.841 | −2.001 | 53.361 | 1.00 | 47.07 | B |
| ATOM | 2504 | CA | TRP B | 448 | 73.278 | −2.349 | 52.058 | 1.00 | 46.70 | B |
| ATOM | 2505 | CB | TRP B | 448 | 71.807 | −2.704 | 52.201 | 1.00 | 43.15 | B |
| ATOM | 2506 | CG | TRP B | 448 | 71.506 | −3.423 | 53.437 | 1.00 | 40.87 | B |
| ATOM | 2507 | CD2 | TRP B | 448 | 71.126 | −4.793 | 53.554 | 1.00 | 39.93 | B |
| ATOM | 2508 | CE2 | TRP B | 448 | 70.984 | −5.067 | 54.925 | 1.00 | 39.42 | B |
| ATOM | 2509 | CE3 | TRP B | 448 | 70.907 | −5.820 | 52.635 | 1.00 | 38.21 | B |
| ATOM | 2510 | CD1 | TRP B | 448 | 71.565 | −2.930 | 54.698 | 1.00 | 40.70 | B |
| ATOM | 2511 | NE1 | TRP B | 448 | 71.253 | −3.910 | 55.603 | 1.00 | 40.18 | B |
| ATOM | 2512 | CZ2 | TRP B | 448 | 70.620 | −6.324 | 55.400 | 1.00 | 39.77 | B |
| ATOM | 2513 | CZ3 | TRP B | 448 | 70.547 | −7.073 | 53.110 | 1.00 | 38.97 | B |
| ATOM | 2514 | CH2 | TRP B | 448 | 70.413 | −7.314 | 54.480 | 1.00 | 38.38 | B |
| ATOM | 2515 | C | TRP B | 448 | 73.383 | −1.223 | 51.038 | 1.00 | 48.93 | B |
| ATOM | 2516 | O | TRP B | 448 | 73.774 | −0.096 | 51.360 | 1.00 | 49.67 | B |
| ATOM | 2517 | N | ASP B | 449 | 73.001 | −1.542 | 49.803 | 1.00 | 51.05 | B |
| ATOM | 2518 | CA | ASP B | 449 | 73.014 | −0.586 | 48.698 | 1.00 | 52.47 | B |
| ATOM | 2519 | CB | ASP B | 449 | 74.247 | −0.830 | 47.826 | 1.00 | 53.58 | B |
| ATOM | 2520 | CG | ASP B | 449 | 74.259 | 0.024 | 46.568 | 1.00 | 55.71 | B |
| ATOM | 2521 | OD1 | ASP B | 449 | 73.432 | −0.234 | 45.662 | 1.00 | 56.14 | B |
| ATOM | 2522 | OD2 | ASP B | 449 | 75.095 | 0.954 | 46.489 | 1.00 | 55.67 | B |
| ATOM | 2523 | C | ASP B | 449 | 71.733 | −0.750 | 47.867 | 1.00 | 52.24 | B |
| ATOM | 2524 | O | ASP B | 449 | 71.541 | −1.777 | 47.225 | 1.00 | 52.13 | B |
| ATOM | 2525 | N | ILE B | 450 | 70.864 | 0.260 | 47.883 | 1.00 | 52.28 | B |
| ATOM | 2526 | CA | ILE B | 450 | 69.607 | 0.199 | 47.135 | 1.00 | 52.23 | B |
| ATOM | 2527 | CB | ILE B | 450 | 68.729 | 1.477 | 47.359 | 1.00 | 51.25 | B |
| ATOM | 2528 | CG2 | ILE B | 450 | 67.411 | 1.352 | 46.609 | 1.00 | 50.40 | B |
| ATOM | 2529 | CG1 | ILE B | 450 | 68.430 | 1.658 | 48.851 | 1.00 | 50.03 | B |
| ATOM | 2530 | CO1 | ILE B | 450 | 67.617 | 2.890 | 49.180 | 1.00 | 49.00 | B |
| ATOM | 2531 | C | ILE B | 450 | 69.849 | 0.024 | 45.639 | 1.00 | 52.47 | B |
| ATOM | 2532 | O | ILE B | 450 | 69.266 | −0.860 | 45.024 | 1.00 | 52.87 | B |
| ATOM | 2533 | N | LYS B | 451 | 70.705 | 0.863 | 45.060 | 1.00 | 53.03 | B |
| ATOM | 2534 | CA | LYS B | 451 | 71.008 | 0.779 | 43.638 | 1.00 | 53.15 | B |
| ATOM | 2535 | CB | LYS B | 451 | 72.346 | 1.458 | 43.339 | 1.00 | 53.71 | B |
| ATOM | 2536 | CG | LYS B | 451 | 72.315 | 2.978 | 43.471 | 0.00 | 54.77 | B |
| ATOM | 2537 | CD | LYS B | 451 | 71.228 | 3.578 | 42.581 | 0.00 | 55.27 | B |
| ATOM | 2538 | CE | LYS B | 451 | 71.190 | 5.104 | 42.643 | 0.00 | 55.45 | B |
| ATOM | 2539 | NZ | LYS B | 451 | 72.286 | 5.761 | 41.877 | 0.00 | 55.61 | B |
| ATOM | 2540 | C | LYS B | 451 | 71.059 | −0.684 | 43.212 | 1.00 | 53.22 | B |
| ATOM | 2541 | O | LYS B | 451 | 70.241 | −1.139 | 42.415 | 1.00 | 51.85 | B |
| ATOM | 2542 | N | LYS B | 452 | 72.007 | −1.428 | 43.765 | 1.00 | 54.30 | B |
| ATOM | 2543 | CA | LYS B | 452 | 72.138 | −2.833 | 43.431 | 1.00 | 55.55 | B |
| ATOM | 2544 | CB | LYS B | 452 | 73.549 | −3.321 | 43.756 | 1.00 | 56.49 | B |
| ATOM | 2545 | CG | LYS B | 452 | 74.596 | −2.840 | 42.773 | 0.00 | 57.18 | B |
| ATOM | 2546 | CD | LYS B | 452 | 74.345 | −3.420 | 41.393 | 0.00 | 57.78 | B |
| ATOM | 2547 | CE | LYS B | 452 | 75.353 | −2.896 | 40.391 | 0.00 | 58.13 | B |
| ATOM | 2548 | NZ | LYS B | 452 | 75.066 | −3.409 | 39.027 | 0.00 | 58.45 | B |
| ATOM | 2549 | C | LYS B | 452 | 71.113 | −3.701 | 44.147 | 1.00 | 56.44 | B |
| ATOM | 2550 | O | LYS B | 452 | 70.797 | −4.790 | 43.679 | 1.00 | 58.17 | B |
| ATOM | 2551 | N | GLY B | 453 | 70.594 | −3.234 | 45.279 | 1.00 | 56.96 | B |
| ATOM | 2552 | CA | GLY B | 453 | 69.605 | −4.013 | 46.013 | 1.00 | 57.08 | B |
| ATOM | 2553 | C | GLY B | 453 | 70.167 | −5.275 | 46.650 | 1.00 | 57.02 | B |
| ATOM | 2554 | O | GLY B | 453 | 69.573 | −6.353 | 46.568 | 1.00 | 56.26 | B |
| ATOM | 2555 | N | CYS B | 454 | 71.319 | −5.143 | 47.297 | 1.00 | 57.42 | B |
| ATOM | 2556 | CA | CYS B | 454 | 71.958 | −6.281 | 47.945 | 1.00 | 57.82 | B |
| ATOM | 2557 | CB | CYS B | 454 | 72.844 | −7.024 | 46.937 | 1.00 | 59.05 | B |
| ATOM | 2558 | SG | CYS B | 454 | 74.118 | −5.986 | 46.135 | 1.00 | 59.80 | B |
| ATOM | 2559 | C | GY5 B | 454 | 72.800 | −5.834 | 49.136 | 1.00 | 57.12 | B |
| ATOM | 2560 | O | CYS B | 454 | 73.084 | −4.644 | 49.300 | 1.00 | 57.22 | B |
| ATOM | 2561 | N | CYS B | 455 | 73.196 | −6.798 | 49.961 | 1.00 | 55.66 | B |
| ATOM | 2562 | CA | CYS B | 455 | 74.021 | −6.529 | 51.136 | 1.00 | 54.95 | B |
| ATOM | 2563 | CB | CYS B | 455 | 73.831 | −7.667 | 52.140 | 1.00 | 54.92 | B |
| ATOM | 2564 | SG | GY5 B | 455 | 74.504 | −7.375 | 53.769 | 1.00 | 58.03 | B |
| ATOM | 2565 | C | GY5 B | 455 | 75.485 | −6.436 | 50.678 | 1.00 | 54.21 | B |
| ATOM | 2566 | O | CYS B | 455 | 76.024 | −7.404 | 50.146 | 1.00 | 55.39 | B |
| ATOM | 2567 | N | THR B | 456 | 76.120 | −5.277 | 50.875 | 1.00 | 52.48 | B |
| ATOM | 2568 | CA | THR B | 456 | 77.514 | −5.074 | 50.446 | 1.00 | 50.81 | B |
| ATOM | 2569 | CB | THR B | 456 | 77.771 | −3.609 | 49.987 | 1.00 | 50.32 | B |
| ATOM | 2570 | OG1 | THR B | 456 | 77.631 | −2.712 | 51.095 | 1.00 | 50.40 | B |
| ATOM | 2571 | CG2 | THR B | 456 | 76.808 | −3.217 | 48.894 | 1.00 | 49.71 | B |
| ATOM | 2572 | C | THR B | 456 | 78.608 | −5.434 | 51.472 | 1.00 | 49.16 | B |
| ATOM | 2573 | O | THR B | 456 | 79.773 | −5.613 | 51.109 | 1.00 | 49.26 | B |
| ATOM | 2574 | N | HIS B | 457 | 78.242 | −5.537 | 52.743 | 1.00 | 46.55 | B |
| ATOM | 2575 | CA | HIS B | 457 | 79.205 | −5.871 | 53.780 | 1.00 | 43.83 | B |
| ATOM | 2576 | CB | HIS B | 457 | 79.956 | −4.615 | 54.229 | 1.00 | 43.71 | B |
| ATOM | 2577 | CG | HIS B | 457 | 80.731 | −3.947 | 53.136 | 1.00 | 44.34 | B |
| ATOM | 2578 | CD2 | HIS B | 457 | 80.557 | −2.753 | 52.524 | 1.00 | 44.60 | B |
| ATOM | 2579 | ND1 | HIS B | 457 | 81.832 | −4.524 | 52.543 | 1.00 | 44.61 | B |
| ATOM | 2580 | CE1 | HIS B | 457 | 82.305 | −3.711 | 51.615 | 1.00 | 44.83 | B |
| ATOM | 2581 | NE2 | HIS B | 457 | 81.550 | −2.630 | 51.584 | 1.00 | 43.67 | B |
| ATOM | 2582 | C | HIS B | 457 | 78.512 | −6.512 | 54.979 | 1.00 | 42.72 | B |
| ATOM | 2583 | O | HIS B | 457 | 77.307 | −6.342 | 55.178 | 1.00 | 41.66 | B |
| ATOM | 2584 | N | VAL B | 458 | 79.275 | −7.255 | 55.776 | 1.00 | 42.43 | B |
| ATOM | 2585 | CA | VAL B | 458 | 78.725 | −7.918 | 56.958 | 1.00 | 40.96 | B |
| ATOM | 2586 | CB | VAL B | 458 | 78.386 | −9.383 | 56.655 | 1.00 | 41.12 | B |
| ATOM | 2587 | CG1 | VAL B | 458 | 77.331 | −9.883 | 57.628 | 1.00 | 39.96 | B |
| ATOM | 2588 | CG2 | VAL B | 458 | 77.913 | −9.519 | 55.225 | 1.00 | 40.46 | B |
| ATOM | 2589 | C | VAL B | 458 | 79.711 | −7.852 | 58.130 | 1.00 | 39.97 | B |
| ATOM | 2590 | O | VAL B | 458 | 80.391 | −8.820 | 58.456 | 1.00 | 38.56 | B |
| ATOM | 2591 | N | PHE B | 459 | 79.744 | −6.690 | 58.768 | 1.00 | 40.60 | B |
| ATOM | 2592 | CA | PHE B | 459 | 80.623 | −6.413 | 59.892 | 1.00 | 41.18 | B |
| ATOM | 2593 | CB | PHE B | 459 | 80.675 | −4.901 | 60.084 | 1.00 | 38.09 | B |
| ATOM | 2594 | CG | PHE B | 459 | 81.196 | −4.158 | 58.881 | 1.00 | 36.10 | B |
| ATOM | 2595 | CD1 | PHE B | 459 | 81.182 | −2.765 | 58.850 | 1.00 | 34.95 | B |
| ATOM | 2596 | CD2 | PHE B | 459 | 81.767 | −4.847 | 57.813 | 1.00 | 34.29 | B |
| ATOM | 2597 | CE1 | PHE B | 459 | 81.741 | −2.055 | 57.773 | 1.00 | 34.55 | B |
| ATOM | 2598 | CE2 | PHE B | 459 | 82.328 | −4.160 | 56.734 | 1.00 | 35.73 | B |
| ATOM | 2599 | CZ | PHE B | 459 | 82.317 | −2.755 | 56.713 | 1.00 | 36.03 | B |
| ATOM | 2600 | C | PHE B | 459 | 80.237 | −7.104 | 61.209 | 1.00 | 43.45 | B |
| ATOM | 2601 | O | PHE B | 459 | 79.357 | −6.630 | 61.921 | 1.00 | 44.50 | B |
| ATOM | 2602 | N | GLU B | 460 | 80.900 | −8.216 | 61.538 | 1.00 | 46.24 | B |
| ATOM | 2603 | CA | GLU B | 460 | 80.608 | −8.939 | 62.783 | 1.00 | 47.21 | B |
| ATOM | 2604 | CB | GLU B | 460 | 80.750 | −10.456 | 62.599 | 1.00 | 46.96 | B |
| ATOM | 2605 | CG | GLU B | 460 | 79.920 | −11.030 | 61.473 | 1.00 | 51.03 | B |
| ATOM | 2606 | CD | GLU B | 460 | 79.971 | −12.553 | 61.386 | 1.00 | 52.17 | B |
| ATOM | 2607 | OE1 | GLU B | 460 | 81.079 | −13.110 | 61.261 | 1.00 | 54.35 | B |
| ATOM | 2608 | OE2 | GLU B | 460 | 78.897 | −13.194 | 61.428 | 1.00 | 51.82 | B |
| ATOM | 2609 | C | GLU B | 460 | 81.532 | −8.491 | 63.909 | 1.00 | 47.31 | B |
| ATOM | 2610 | O | GUI B | 460 | 82.567 | −7.863 | 63.674 | 1.00 | 45.71 | B |
| ATOM | 2611 | N | GLY B | 461 | 81.147 | −8.835 | 65.134 | 1.00 | 48.17 | B |
| ATOM | 2612 | CA | GLY B | 461 | 81.928 | −8.456 | 66.292 | 1.00 | 49.82 | B |
| ATOM | 2613 | C | GLY B | 461 | 81.102 | −8.363 | 67.561 | 1.00 | 50.83 | B |
| ATOM | 2614 | O | GLY B | 461 | 81.379 | −9.074 | 68.529 | 1.00 | 50.20 | B |
| ATOM | 2615 | N | HIS B | 462 | 80.086 | −7.501 | 67.565 | 1.00 | 51.06 | B |
| ATOM | 2616 | CA | HIS B | 462 | 79.251 | −7.346 | 68.750 | 1.00 | 51.73 | B |
| ATOM | 2617 | CB | HIS B | 462 | 78.010 | −6.487 | 68.443 | 1.00 | 50.07 | B |
| ATOM | 2618 | CG | HIS B | 462 | 78.284 | −5.011 | 68.421 | 1.00 | 48.90 | B |
| ATOM | 2619 | CD2 | HIS B | 462 | 78.451 | −4.151 | 67.388 | 1.00 | 48.64 | B |
| ATOM | 2620 | ND1 | HIS B | 462 | 78.443 | −4.264 | 69.568 | 1.00 | 49.43 | B |
| ATOM | 2621 | CE1 | HIS B | 462 | 78.697 | −3.009 | 69.242 | 1.00 | 50.51 | B |
| ATOM | 2622 | NE2 | HIS B | 462 | 78.708 | −2.914 | 67.925 | 1.00 | 48.70 | B |
| ATOM | 2623 | C | HIS B | 462 | 78.846 | −8.707 | 69.315 | 1.00 | 52.82 | B |
| ATOM | 2624 | O | HIS B | 462 | 78.076 | −9.459 | 68.704 | 1.00 | 53.62 | B |
| ATOM | 2625 | N | ASN B | 463 | 79.394 | −9.013 | 70.490 | 1.00 | 53.69 | B |
| ATOM | 2626 | CA | ASN B | 463 | 79.140 | −10.269 | 71.186 | 1.00 | 53.67 | B |
| ATOM | 2627 | CB | ASN B | 463 | 80.119 | −10.413 | 72.369 | 0.00 | 53.95 | B |
| ATOM | 2628 | CG | ASN B | 463 | 81.588 | −10.299 | 71.945 | 1.00 | 54.43 | B |
| ATOM | 2629 | OD1 | ASN B | 463 | 82.173 | −11.235 | 71.378 | 1.00 | 55.63 | B |
| ATOM | 2630 | ND2 | ASN B | 463 | 82.184 | −9.140 | 72.216 | 1.00 | 52.98 | B |
| ATOM | 2631 | C | ASN B | 463 | 77.699 | −10.312 | 71.686 | 1.00 | 53.05 | B |
| ATOM | 2632 | O | ASN B | 463 | 77.353 | −11.133 | 72.525 | 1.00 | 53.74 | B |
| ATOM | 2633 | N | SER B | 464 | 76.867 | −9.414 | 71.162 | 1.00 | 53.52 | B |
| ATOM | 2634 | CA | SER B | 464 | 75.455 | −9.330 | 71.540 | 1.00 | 51.56 | B |
| ATOM | 2635 | CB | SER B | 464 | 75.324 | −8.732 | 72.946 | 1.00 | 52.15 | B |
| ATOM | 2636 | OG | SER B | 464 | 74.010 | −8.925 | 73.444 | 1.00 | 54.03 | B |
| ATOM | 2637 | C | SER B | 464 | 74.645 | −8.487 | 70.527 | 1.00 | 49.85 | B |
| ATOM | 2638 | O | SER B | 464 | 75.210 | −7.852 | 69.622 | 1.00 | 47.81 | B |
| ATOM | 2639 | N | THR B | 465 | 73.321 | −8.496 | 70.695 | 1.00 | 48.25 | B |
| ATOM | 2640 | CA | THR B | 465 | 72.390 | −7.767 | 69.824 | 1.00 | 47.13 | B |
| ATOM | 2641 | CB | THR B | 465 | 70.924 | −7.847 | 70.344 | 1.00 | 48.31 | B |
| ATOM | 2642 | OG1 | THR B | 465 | 70.510 | −9.214 | 70.418 | 1.00 | 51.43 | B |
| ATOM | 2643 | CG2 | THR B | 465 | 69.971 | −7.098 | 69.412 | 1.00 | 49.57 | B |
| ATOM | 2644 | C | THR B | 465 | 72.734 | −6.292 | 69.669 | 1.00 | 45.19 | B |
| ATOM | 2645 | O | THR B | 465 | 73.067 | −5.617 | 70.643 | 1.00 | 45.46 | B |
| ATOM | 2646 | N | VAL B | 466 | 72.636 | −5.804 | 68.433 | 1.00 | 42.21 | B |
| ATOM | 2647 | CA | VAL B | 466 | 72.912 | −4.406 | 68.125 | 1.00 | 39.22 | B |
| ATOM | 2648 | CB | VAL B | 466 | 73.293 | −4.241 | 66.643 | 1.00 | 38.21 | B |
| ATOM | 2649 | CG1 | VAL B | 466 | 73.798 | −2.829 | 66.378 | 1.00 | 37.18 | B |
| ATOM | 2650 | CG2 | VAL B | 466 | 74.354 | −5.264 | 66.285 | 1.00 | 35.99 | B |
| ATOM | 2651 | C | VAL B | 466 | 71.626 | −3.638 | 68.430 | 1.00 | 37.88 | B |
| ATOM | 2652 | O | VAL B | 466 | 70.655 | −3.738 | 67.691 | 1.00 | 35.76 | B |
| ATOM | 2653 | N | ARG B | 467 | 71.623 | −2.886 | 69.530 | 1.00 | 37.28 | B |
| ATOM | 2654 | CA | ARG B | 467 | 70.440 | −2.145 | 69.938 | 1.00 | 36.20 | B |
| ATOM | 2655 | CB | ARG B | 467 | 70.399 | −1.987 | 71.468 | 1.00 | 36.76 | B |
| ATOM | 2656 | CG | ARG B | 467 | 69.038 | −1.513 | 72.012 | 1.00 | 36.98 | B |
| ATOM | 2657 | CD | ARG B | 467 | 67.945 | −2.559 | 71.762 | 1.00 | 39.61 | B |
| ATOM | 2658 | NE | ARG B | 467 | 66.563 | −2.103 | 71.979 | 1.00 | 40.08 | B |
| ATOM | 2659 | CZ | ARG B | 467 | 65.928 | −1.213 | 71.220 | 1.00 | 38.58 | B |
| ATOM | 2660 | NH1 | ARG B | 467 | 66.539 | −0.654 | 70.184 | 1.00 | 36.43 | B |
| ATOM | 2661 | NH2 | ARG B | 467 | 64.664 | −0.913 | 71.476 | 1.00 | 39.24 | B |
| ATOM | 2662 | C | ARG B | 467 | 70.314 | −0.773 | 69.283 | 1.00 | 35.21 | B |
| ATOM | 2663 | O | ARG B | 467 | 69.225 | −0.213 | 69.247 | 1.00 | 36.67 | B |
| ATOM | 2664 | N | CYS B | 468 | 71.400 | −0.222 | 68.758 | 1.00 | 33.04 | B |
| ATOM | 2665 | CA | CYS B | 468 | 71.304 | 1.088 | 68.127 | 1.00 | 33.09 | B |
| ATOM | 2666 | CB | CYS B | 468 | 71.069 | 2.167 | 69.186 | 1.00 | 32.72 | B |
| ATOM | 2667 | SG | CYS B | 468 | 72.414 | 2.394 | 70.339 | 1.00 | 34.24 | B |
| ATOM | 2668 | C | CYS B | 468 | 72.533 | 1.406 | 67.292 | 1.00 | 32.45 | B |
| ATOM | 2669 | O | CYS B | 468 | 73.473 | 0.618 | 67.263 | 1.00 | 33.24 | B |
| ATOM | 2670 | N | LEU B | 469 | 72.522 | 2.546 | 66.605 | 1.00 | 31.75 | B |
| ATOM | 2671 | CA | LEU B | 469 | 73.643 | 2.908 | 65.747 | 1.00 | 32.36 | B |
| ATOM | 2672 | CB | LEU B | 469 | 73.750 | 1.917 | 64.576 | 1.00 | 32.24 | B |
| ATOM | 2673 | CG | LEU B | 469 | 72.822 | 2.067 | 63.362 | 1.00 | 31.67 | B |
| ATOM | 2674 | CD1 | LEU B | 469 | 73.563 | 2.673 | 62.174 | 1.00 | 33.01 | B |
| ATOM | 2675 | CD2 | LEU B | 469 | 72.311 | 0.704 | 62.982 | 1.00 | 33.19 | B |
| ATOM | 2676 | C | LEU B | 469 | 73.491 | 4.309 | 65.179 | 1.00 | 32.21 | B |
| ATOM | 2677 | O | LEU B | 469 | 72.434 | 4.910 | 65.285 | 1.00 | 32.62 | B |
| ATOM | 2678 | N | ASP B | 470 | 74.552 | 4.833 | 64.581 | 1.00 | 33.12 | B |
| ATOM | 2679 | CA | ASP B | 470 | 74.481 | 6.149 | 63.972 | 1.00 | 34.33 | B |
| ATOM | 2680 | CB | ASP B | 470 | 74.431 | 7.223 | 65.055 | 1.00 | 33.47 | B |
| ATOM | 2681 | CG | ASP B | 470 | 74.098 | 8.585 | 64.501 | 1.00 | 32.01 | B |
| ATOM | 2682 | OD1 | ASP B | 470 | 73.302 | 8.647 | 63.551 | 1.00 | 35.18 | B |
| ATOM | 2683 | OD2 | ASP B | 470 | 74.614 | 9.589 | 65.021 | 1.00 | 29.33 | B |
| ATOM | 2684 | C | ASP B | 470 | 75.704 | 6.330 | 63.083 | 1.00 | 35.86 | B |
| ATOM | 2685 | O | ASP B | 470 | 76.619 | 5.509 | 63.124 | 1.00 | 36.56 | B |
| ATOM | 2686 | N | ILE B | 471 | 75.712 | 7.384 | 62.269 | 1.00 | 37.44 | B |
| ATOM | 2687 | CA | ILE B | 471 | 76.841 | 7.663 | 61.380 | 1.00 | 37.88 | B |
| ATOM | 2688 | CB | ILE B | 471 | 76.465 | 7.465 | 59.889 | 1.00 | 36.84 | B |
| ATOM | 2689 | CG2 | ILE B | 471 | 77.698 | 7.683 | 59.005 | 1.00 | 36.56 | B |
| ATOM | 2690 | CG1 | ILE B | 471 | 75.942 | 6.050 | 59.649 | 1.00 | 36.63 | B |
| ATOM | 2691 | CD1 | ILE B | 471 | 75.305 | 5.868 | 58.283 | 1.00 | 35.99 | B |
| ATOM | 2692 | C | ILE B | 471 | 77.309 | 9.106 | 61.569 | 1.00 | 38.95 | B |
| ATOM | 2693 | O | ILE B | 471 | 76.493 | 10.013 | 61.769 | 1.00 | 39.67 | B |
| ATOM | 2694 | N | VAL B | 472 | 78.625 | 9.305 | 61.522 | 1.00 | 39.62 | B |
| ATOM | 2695 | CA | VAL B | 472 | 79.219 | 10.631 | 61.670 | 1.00 | 40.41 | B |
| ATOM | 2696 | CB | VAL B | 472 | 79.932 | 10.786 | 63.018 | 1.00 | 39.84 | B |
| ATOM | 2697 | CG1 | VAL B | 472 | 78.967 | 10.468 | 64.158 | 1.00 | 37.97 | B |
| ATOM | 2698 | CG2 | VAL B | 472 | 81.150 | 9.862 | 63.066 | 1.00 | 38.84 | B |
| ATOM | 2699 | C | VAL B | 472 | 80.255 | 10.802 | 60.579 | 1.00 | 42.22 | B |
| ATOM | 2700 | O | VAL B | 472 | 80.780 | 9.820 | 60.064 | 1.00 | 41.41 | B |
| ATOM | 2701 | N | GLU B | 473 | 80.544 | 12.048 | 60.227 | 1.00 | 44.76 | B |
| ATOM | 2702 | CA | GLU B | 473 | 81.539 | 12.329 | 59.207 | 1.00 | 48.54 | B |
| ATOM | 2703 | CB | GLU B | 473 | 80.872 | 12.865 | 57.934 | 1.00 | 49.21 | B |
| ATOM | 2704 | CG | GLU B | 473 | 81.851 | 13.193 | 56.784 | 1.00 | 51.32 | B |
| ATOM | 2705 | CD | GLU B | 473 | 81.143 | 13.514 | 55.465 | 1.00 | 51.78 | B |
| ATOM | 2706 | OE1 | GLU B | 473 | 80.489 | 12.609 | 54.902 | 1.00 | 52.52 | B |
| ATOM | 2707 | OE2 | GLU B | 473 | 81.232 | 14.670 | 54.996 | 1.00 | 52.59 | B |
| ATOM | 2708 | C | GLU B | 473 | 82.568 | 13.329 | 59.737 | 1.00 | 50.91 | B |
| ATOM | 2709 | O | GLU B | 473 | 82.225 | 14.419 | 60.205 | 1.00 | 50.21 | B |
| ATOM | 2710 | N | TYR B | 474 | 83.835 | 12.933 | 59.683 | 1.00 | 53.85 | B |
| ATOM | 2711 | CA | TYR B | 474 | 84.927 | 13.775 | 60.151 | 1.00 | 56.31 | B |
| ATOM | 2712 | CB | TYR B | 474 | 85.504 | 13.214 | 61.450 | 1.00 | 57.48 | B |
| ATOM | 2713 | CG | TYR B | 474 | 86.410 | 14.180 | 62.164 | 1.00 | 59.70 | B |
| ATOM | 2714 | CD1 | TYR B | 474 | 85.925 | 15.408 | 62.613 | 1.00 | 60.47 | B |
| ATOM | 2715 | CE1 | TYR B | 474 | 86.744 | 16.305 | 63.285 | 1.00 | 60.97 | B |
| ATOM | 2716 | CD2 | TYR B | 474 | 87.749 | 13.870 | 62.402 | 1.00 | 60.68 | B |
| ATOM | 2717 | CE2 | TYR B | 474 | 88.581 | 14.763 | 63.075 | 1.00 | 61.50 | B |
| ATOM | 2718 | CZ | TYR B | 474 | 88.068 | 15.978 | 63.514 | 1.00 | 61.92 | B |
| ATOM | 2719 | OH | TYR B | 474 | 88.875 | 16.861 | 64.194 | 1.00 | 63.90 | B |
| ATOM | 2720 | C | TYR B | 474 | 86.001 | 13.795 | 59.075 | 1.00 | 56.54 | B |
| ATOM | 2721 | O | TYR B | 474 | 86.293 | 12.768 | 58.460 | 1.00 | 56.18 | B |
| ATOM | 2722 | N | LYS B | 475 | 86.589 | 14.966 | 58.856 | 1.00 | 58.01 | B |
| ATOM | 2723 | CA | LYS B | 475 | 87.623 | 15.128 | 57.840 | 1.00 | 59.54 | B |
| ATOM | 2724 | CB | LYS B | 475 | 88.958 | 14.579 | 58.351 | 1.00 | 60.12 | B |
| ATOM | 2725 | CG | LYS B | 475 | 89.453 | 15.213 | 59.653 | 1.00 | 60.74 | B |
| ATOM | 2726 | CD | LYS B | 475 | 89.934 | 16.642 | 59.449 | 1.00 | 61.48 | B |
| ATOM | 2727 | CE | LYS B | 475 | 90.341 | 17.283 | 60.765 | 0.00 | 61.92 | B |
| ATOM | 2728 | NZ | LYS B | 475 | 90.797 | 18.687 | 60.568 | 0.00 | 62.37 | B |
| ATOM | 2729 | C | LYS B | 475 | 87.200 | 14.393 | 56.563 | 1.00 | 60.31 | B |
| ATOM | 2730 | O | LYS B | 475 | 87.882 | 13.481 | 56.093 | 1.00 | 61.02 | B |
| ATOM | 2731 | N | ASN B | 476 | 86.057 | 14.796 | 56.020 | 1.00 | 60.89 | B |
| ATOM | 2732 | CA | ASN B | 476 | 85.523 | 14.199 | 54.813 | 1.00 | 60.87 | B |
| ATOM | 2733 | CB | ASN B | 476 | 86.260 | 14.759 | 53.594 | 1.00 | 63.50 | B |
| ATOM | 2734 | CG | ASN B | 476 | 85.792 | 16.172 | 53.224 | 1.00 | 66.46 | B |
| ATOM | 2735 | OD1 | ASN B | 476 | 84.649 | 16.361 | 52.785 | 1.00 | 68.68 | B |
| ATOM | 2736 | ND2 | ASN B | 476 | 86.668 | 17.167 | 53.405 | 1.00 | 65.78 | B |
| ATOM | 2737 | C | ASN B | 476 | 85.593 | 12.676 | 54.843 | 1.00 | 60.30 | B |
| ATOM | 2738 | O | ASN B | 476 | 85.828 | 12.036 | 53.822 | 1.00 | 61.52 | B |
| ATOM | 2739 | N | ILE B | 477 | 85.379 | 12.095 | 56.018 | 1.00 | 58.81 | B |
| ATOM | 2740 | CA | ILE B | 477 | 85.395 | 10.642 | 56.155 | 1.00 | 57.40 | B |
| ATOM | 2741 | CB | ILE B | 477 | 86.720 | 10.148 | 56.780 | 1.00 | 58.27 | B |
| ATOM | 2742 | CG2 | ILE B | 477 | 86.741 | 8.618 | 56.804 | 1.00 | 58.03 | B |
| ATOM | 2743 | CG1 | ILE B | 477 | 87.903 | 10.659 | 55.960 | 0.00 | 58.14 | B |
| ATOM | 2744 | CD1 | ILE B | 477 | 89.253 | 10.422 | 56.599 | 0.00 | 58.25 | B |
| ATOM | 2745 | C | ILE B | 477 | 84.224 | 10.183 | 57.030 | 1.00 | 55.59 | B |
| ATOM | 2746 | O | ILE B | 477 | 84.050 | 10.661 | 58.151 | 1.00 | 55.65 | B |
| ATOM | 2747 | N | LYS B | 478 | 83.423 | 9.257 | 56.515 | 1.00 | 52.92 | B |
| ATOM | 2748 | CA | LYS B | 478 | 82.281 | 8.760 | 57.264 | 1.00 | 51.69 | B |
| ATOM | 2749 | CB | LYS B | 478 | 81.106 | 8.479 | 56.322 | 1.00 | 50.04 | B |
| ATOM | 2750 | CG | LYS B | 478 | 80.324 | 9.717 | 55.899 | 1.00 | 47.12 | B |
| ATOM | 2751 | CD | LYS B | 478 | 78.972 | 9.329 | 55.324 | 1.00 | 46.70 | B |
| ATOM | 2752 | CE | LYS B | 478 | 78.106 | 10.553 | 55.048 | 1.00 | 45.79 | B |
| ATOM | 2753 | NZ | LYS B | 478 | 76.681 | 10.181 | 54.803 | 1.00 | 44.56 | B |
| ATOM | 2754 | C | LYS B | 478 | 82.591 | 7.509 | 58.090 | 1.00 | 52.07 | B |
| ATOM | 2755 | O | LYS B | 478 | 83.279 | 6.593 | 57.620 | 1.00 | 53.19 | B |
| ATOM | 2756 | N | TYR B | 479 | 82.076 | 7.485 | 59.323 | 1.00 | 51.09 | B |
| ATOM | 2757 | CA | TYR B | 479 | 82.273 | 6.363 | 60.250 | 1.00 | 49.59 | B |
| ATOM | 2758 | CB | TYR B | 479 | 83.083 | 6.815 | 61.492 | 1.00 | 50.00 | B |
| ATOM | 2759 | CG | TYR B | 479 | 84.438 | 7.442 | 61.183 | 1.00 | 49.78 | B |
| ATOM | 2760 | CD1 | TYR B | 479 | 84.526 | 8.594 | 60.402 | 1.00 | 50.71 | B |
| ATOM | 2761 | CE1 | TYR B | 479 | 85.754 | 9.165 | 60.062 | 1.00 | 49.52 | B |
| ATOM | 2762 | CD2 | TYR B | 479 | 85.629 | 6.869 | 61.630 | 1.00 | 49.70 | B |
| ATOM | 2763 | CE2 | TYR B | 479 | 86.870 | 7.438 | 61.291 | 1.00 | 49.93 | B |
| ATOM | 2764 | CZ | TYR B | 479 | 86.918 | 8.586 | 60.502 | 1.00 | 49.57 | B |
| ATOM | 2765 | OH | TYR B | 479 | 88.115 | 9.155 | 60.126 | 1.00 | 48.42 | B |
| ATOM | 2766 | C | TYR B | 479 | 80.922 | 5.790 | 60.698 | 1.00 | 48.39 | B |
| ATOM | 2767 | O | TYR B | 479 | 79.877 | 6.381 | 60.463 | 1.00 | 47.95 | B |
| ATOM | 2768 | N | ILE B | 480 | 80.954 | 4.629 | 61.337 | 1.00 | 48.14 | B |
| ATOM | 2769 | CA | ILE B | 480 | 79.742 | 3.986 | 61.828 | 1.00 | 47.98 | B |
| ATOM | 2770 | CB | ILE B | 480 | 79.470 | 2.669 | 61.090 | 1.00 | 47.82 | B |
| ATOM | 2771 | CG2 | ILE B | 480 | 78.250 | 1.983 | 61.679 | 1.00 | 48.23 | B |
| ATOM | 2772 | CG1 | ILE B | 480 | 79.275 | 2.929 | 59.599 | 1.00 | 47.92 | B |
| ATOM | 2773 | CD1 | ILE B | 480 | 79.279 | 1.675 | 58.768 | 1.00 | 46.66 | B |
| ATOM | 2774 | C | ILE B | 480 | 79.930 | 3.651 | 63.308 | 1.00 | 48.77 | B |
| ATOM | 2775 | O | ILE B | 480 | 80.961 | 3.089 | 63.699 | 1.00 | 49.54 | B |
| ATOM | 2776 | N | VAL B | 481 | 78.931 | 3.982 | 64.124 | 1.00 | 48.38 | B |
| ATOM | 2777 | CA | VAL B | 481 | 78.975 | 3.718 | 65.566 | 1.00 | 46.50 | B |
| ATOM | 2778 | CB | VAL B | 481 | 78.917 | 5.041 | 66.382 | 1.00 | 46.27 | B |
| ATOM | 2779 | CG1 | VAL B | 481 | 79.048 | 4.749 | 67.871 | 1.00 | 44.39 | B |
| ATOM | 2780 | CG2 | VAL B | 481 | 80.004 | 5.990 | 65.914 | 1.00 | 45.50 | B |
| ATOM | 2781 | C | VAL B | 481 | 77.778 | 2.855 | 65.962 | 1.00 | 45.57 | B |
| ATOM | 2782 | O | VAL B | 481 | 76.634 | 3.281 | 65.823 | 1.00 | 45.94 | B |
| ATOM | 2783 | N | THR B | 482 | 78.039 | 1.652 | 66.460 | 1.00 | 43.78 | B |
| ATOM | 2784 | CA | THR B | 482 | 76.963 | 0.752 | 66.865 | 1.00 | 43.35 | B |
| ATOM | 2785 | CB | THR B | 482 | 77.050 | −0.608 | 66.091 | 1.00 | 44.57 | B |
| ATOM | 2786 | OG1 | THR B | 482 | 78.422 | −0.943 | 65.851 | 1.00 | 44.68 | B |
| ATOM | 2787 | CG2 | THR B | 482 | 76.315 | −0.523 | 64.748 | 1.00 | 44.18 | B |
| ATOM | 2788 | C | THR B | 482 | 76.946 | 0.475 | 68.379 | 1.00 | 42.32 | B |
| ATOM | 2789 | O | THR B | 482 | 77.979 | 0.184 | 68.977 | 1.00 | 42.34 | B |
| ATOM | 2790 | N | GLY B | 483 | 75.769 | 0.583 | 68.991 | 1.00 | 41.18 | B |
| ATOM | 2791 | CA | GLY B | 483 | 75.643 | 0.319 | 70.410 | 1.00 | 42.21 | B |
| ATOM | 2792 | C | GLY B | 483 | 75.021 | −1.057 | 70.556 | 1.00 | 43.25 | B |
| ATOM | 2793 | O | GLY B | 483 | 74.069 | −1.385 | 69.837 | 1.00 | 44.15 | B |
| ATOM | 2794 | N | SER B | 484 | 75.536 | −1.865 | 71.479 | 1.00 | 42.55 | B |
| ATOM | 2795 | CA | SER B | 484 | 75.018 | −3.216 | 71.650 | 1.00 | 42.61 | B |
| ATOM | 2796 | CB | SER B | 484 | 76.015 | −4.236 | 71.071 | 1.00 | 42.00 | B |
| ATOM | 2797 | OG | SER B | 484 | 75.676 | −5.572 | 71.425 | 1.00 | 39.48 | B |
| ATOM | 2798 | C | SER B | 484 | 74.721 | −3.560 | 73.099 | 1.00 | 43.04 | B |
| ATOM | 2799 | O | SER B | 484 | 75.118 | −2.833 | 74.007 | 1.00 | 43.14 | B |
| ATOM | 2800 | N | ARG B | 485 | 73.993 | −4.664 | 73.294 | 1.00 | 43.38 | B |
| ATOM | 2801 | CA | ARG B | 485 | 73.628 | −5.182 | 74.618 | 1.00 | 42.66 | B |
| ATOM | 2802 | CB | ARG B | 485 | 72.532 | −6.233 | 74.484 | 1.00 | 41.35 | B |
| ATOM | 2803 | CG | ARG B | 485 | 71.309 | −5.751 | 73.737 | 1.00 | 41.63 | B |
| ATOM | 2804 | CD | ARG B | 485 | 70.170 | −5.535 | 74.696 | 1.00 | 40.30 | B |
| ATOM | 2805 | NE | ARG B | 485 | 69.101 | −6.499 | 74.462 | 1.00 | 41.39 | B |
| ATOM | 2806 | CZ | ARG B | 485 | 67.859 | −6.154 | 74.146 | 1.00 | 42.42 | B |
| ATOM | 2807 | NH1 | ARG B | 485 | 67.547 | −4.874 | 74.029 | 1.00 | 44.59 | B |
| ATOM | 2808 | NH2 | ARG B | 485 | 66.927 | −7.076 | 73.964 | 1.00 | 41.56 | B |
| ATOM | 2809 | C | ARG B | 485 | 74.879 | −5.837 | 75.200 | 1.00 | 43.12 | B |
| ATOM | 2810 | O | ARG B | 485 | 74.845 | −6.448 | 76.264 | 1.00 | 42.36 | B |
| ATOM | 2811 | N | ASP B | 486 | 75.974 | −5.714 | 74.463 | 1.00 | 43.89 | B |
| ATOM | 2812 | CA | ASP B | 486 | 77.232 | −6.263 | 74.880 | 1.00 | 44.86 | B |
| ATOM | 2813 | CB | ASP B | 486 | 78.100 | −6.572 | 73.646 | 1.00 | 45.64 | B |
| ATOM | 2814 | CG | ASP B | 486 | 78.713 | −5.327 | 73.012 | 1.00 | 46.63 | B |
| ATOM | 2815 | OD1 | ASP B | 486 | 78.016 | −4.298 | 72.888 | 1.00 | 46.93 | B |
| ATOM | 2816 | OD2 | ASP B | 486 | 79.897 | −5.387 | 72.619 | 1.00 | 47.67 | B |
| ATOM | 2817 | C | ASP B | 486 | 77.887 | −5.242 | 75.807 | 1.00 | 45.67 | B |
| ATOM | 2818 | O | ASP B | 486 | 78.967 | −5.486 | 76.344 | 1.00 | 46.05 | B |
| ATOM | 2819 | N | ASN B | 487 | 77.207 | −4.108 | 76.003 | 1.00 | 45.49 | B |
| ATOM | 2820 | CA | ASN B | 487 | 77.673 | −3.017 | 76.873 | 1.00 | 44.88 | B |
| ATOM | 2821 | CB | ASN B | 487 | 78.223 | −3.571 | 78.188 | 1.00 | 44.56 | B |
| ATOM | 2822 | CG | ASN B | 487 | 77.171 | −4.261 | 79.021 | 1.00 | 45.76 | B |
| ATOM | 2823 | OD1 | ASN B | 487 | 76.153 | −4.717 | 78.508 | 1.00 | 47.63 | B |
| ATOM | 2824 | ND2 | ASN B | 487 | 77.421 | −4.359 | 80.317 | 1.00 | 45.91 | B |
| ATOM | 2825 | C | ASN B | 487 | 78.748 | −2.146 | 76.242 | 1.00 | 44.09 | B |
| ATOM | 2826 | O | ASN B | 487 | 79.333 | −1.306 | 76.920 | 1.00 | 43.58 | B |
| ATOM | 2827 | N | THR B | 488 | 79.009 | −2.347 | 74.953 | 1.00 | 44.03 | B |
| ATOM | 2828 | CA | THR B | 488 | 80.038 | −1.576 | 74.267 | 1.00 | 45.88 | B |
| ATOM | 2829 | CB | THR B | 488 | 81.264 | −2.458 | 73.895 | 1.00 | 46.72 | B |
| ATOM | 2830 | OG1 | THR B | 488 | 81.026 | −3.098 | 72.633 | 1.00 | 46.03 | B |
| ATOM | 2831 | CG2 | THR B | 488 | 81.498 | −3.545 | 74.958 | 1.00 | 47.35 | B |
| ATOM | 2832 | C | THR B | 488 | 79.525 | −0.958 | 72.971 | 1.00 | 46.36 | B |
| ATOM | 2833 | O | THR B | 488 | 78.403 | −1.224 | 72.542 | 1.00 | 47.28 | B |
| ATOM | 2834 | N | LEU B | 489 | 80.369 | −0.135 | 72.355 | 1.00 | 45.88 | B |
| ATOM | 2835 | CA | LEU B | 489 | 80.053 | 0.511 | 71.090 | 1.00 | 46.03 | B |
| ATOM | 2836 | CB | LEU B | 489 | 79.848 | 2.015 | 71.269 | 1.00 | 45.02 | B |
| ATOM | 2837 | CG | LEU B | 489 | 78.651 | 2.443 | 72.110 | 1.00 | 44.47 | B |
| ATOM | 2838 | CD1 | LEU B | 489 | 79.093 | 2.732 | 73.518 | 1.00 | 43.21 | B |
| ATOM | 2839 | CD2 | LEU B | 489 | 78.011 | 3.655 | 71.484 | 1.00 | 45.05 | B |
| ATOM | 2840 | C | LEU B | 489 | 81.186 | 0.293 | 70.102 | 1.00 | 47.37 | B |
| ATOM | 2841 | O | LEU B | 489 | 82.281 | 0.840 | 70.280 | 1.00 | 48.75 | B |
| ATOM | 2842 | N | HIS B | 490 | 80.930 | −0.507 | 69.067 | 1.00 | 46.28 | B |
| ATOM | 2843 | CA | HIS B | 490 | 81.940 | −0.766 | 68.040 | 1.00 | 44.43 | B |
| ATOM | 2844 | CB | HIS B | 490 | 81.680 | −2.105 | 67.363 | 1.00 | 46.31 | B |
| ATOM | 2845 | CG | HIS B | 490 | 81.979 | −3.288 | 68.226 | 1.00 | 48.42 | B |
| ATOM | 2846 | CD2 | HIS B | 490 | 82.601 | −4.456 | 67.944 | 1.00 | 48.94 | B |
| ATOM | 2847 | ND1 | HIS B | 490 | 81.584 | −3.371 | 69.544 | 1.00 | 48.72 | B |
| ATOM | 2848 | CE1 | HIS B | 490 | 81.947 | −4.542 | 70.035 | 1.00 | 48.74 | B |
| ATOM | 2849 | NE2 | HIS B | 490 | 82.566 | −5.220 | 69.084 | 1.00 | 49.32 | B |
| ATOM | 2850 | C | HIS B | 490 | 81.932 | 0.332 | 66.988 | 1.00 | 41.63 | B |
| ATOM | 2851 | O | HIS B | 490 | 80.873 | 0.804 | 66.598 | 1.00 | 39.88 | B |
| ATOM | 2852 | N | VAL B | 491 | 83.119 | 0.727 | 66.537 | 1.00 | 41.79 | B |
| ATOM | 2853 | CA | VAL B | 491 | 83.268 | 1.774 | 65.523 | 1.00 | 43.45 | B |
| ATOM | 2854 | CB | VAL B | 491 | 84.258 | 2.873 | 65.987 | 1.00 | 43.37 | B |
| ATOM | 2855 | CG1 | VAL B | 491 | 84.565 | 3.838 | 64.843 | 1.00 | 43.14 | B |
| ATOM | 2856 | CG2 | VAL B | 491 | 83.661 | 3.642 | 67.158 | 1.00 | 42.84 | B |
| ATOM | 2857 | C | VAL B | 491 | 83.773 | 1.199 | 64.201 | 1.00 | 43.94 | B |
| ATOM | 2858 | O | VAL B | 491 | 84.794 | 0.515 | 64.165 | 1.00 | 45.06 | B |
| ATOM | 2859 | N | TRP B | 492 | 83.056 | 1.479 | 63.115 | 1.00 | 44.26 | B |
| ATOM | 2860 | CA | TRP B | 492 | 83.453 | 0.982 | 61.804 | 1.00 | 43.81 | B |
| ATOM | 2861 | CB | TRP B | 492 | 82.481 | −0.086 | 61.312 | 1.00 | 42.52 | B |
| ATOM | 2862 | CG | TRP B | 492 | 81.869 | −0.922 | 62.399 | 1.00 | 42.97 | B |
| ATOM | 2863 | CD2 | TRP B | 492 | 82.119 | −2.308 | 62.661 | 1.00 | 43.02 | B |
| ATOM | 2864 | CE2 | TRP B | 492 | 81.258 | −2.696 | 63.721 | 1.00 | 42.64 | B |
| ATOM | 2865 | CE3 | TRP B | 492 | 82.980 | −3.262 | 62.102 | 1.00 | 42.84 | B |
| ATOM | 2866 | CD1 | TRP B | 492 | 80.906 | −0.535 | 63.289 | 1.00 | 42.27 | B |
| ATOM | 2867 | NE1 | TRP B | 492 | 80.532 | −1.593 | 64.081 | 1.00 | 42.22 | B |
| ATOM | 2868 | CZ2 | TRP B | 492 | 81.231 | −4.000 | 64.235 | 1.00 | 43.33 | B |
| ATOM | 2869 | CZ3 | TRP B | 492 | 82.955 | −4.562 | 62.613 | 1.00 | 43.23 | B |
| ATOM | 2870 | CR2 | TRP B | 492 | 82.083 | −4.917 | 63.670 | 1.00 | 43.67 | B |
| ATOM | 2871 | C | TRP B | 492 | 83.493 | 2.123 | 60.804 | 1.00 | 44.22 | B |
| ATOM | 2872 | O | TRP B | 492 | 83.061 | 3.233 | 61.094 | 1.00 | 43.90 | B |
| ATOM | 2873 | N | LYS B | 493 | 84.033 | 1.847 | 59.627 | 1.00 | 45.31 | B |
| ATOM | 2874 | CA | LYS B | 493 | 84.115 | 2.855 | 58.596 | 1.00 | 46.28 | B |
| ATOM | 2875 | CB | LYS B | 493 | 85.515 | 2.896 | 57.997 | 1.00 | 47.20 | B |
| ATOM | 2876 | CG | LYS B | 493 | 86.600 | 3.104 | 59.021 | 1.00 | 48.93 | B |
| ATOM | 2877 | CD | LYS B | 493 | 87.962 | 3.179 | 58.363 | 0.00 | 48.94 | B |
| ATOM | 2878 | CE | LYS B | 493 | 89.061 | 3.374 | 59.394 | 0.00 | 49.30 | B |
| ATOM | 2879 | NZ | LYS B | 493 | 90.390 | 3.550 | 58.746 | 0.00 | 49.52 | B |
| ATOM | 2880 | C | LYS B | 493 | 83.108 | 2.522 | 57.518 | 1.00 | 47.32 | B |
| ATOM | 2881 | O | LYS B | 493 | 82.863 | 1.349 | 57.220 | 1.00 | 47.63 | B |
| ATOM | 2882 | N | LEU B | 494 | 82.514 | 3.563 | 56.952 | 1.00 | 47.99 | B |
| ATOM | 2883 | CA | LEU B | 494 | 81.539 | 3.395 | 55.895 | 1.00 | 48.33 | B |
| ATOM | 2884 | CB | LEU B | 494 | 80.733 | 4.674 | 55.717 | 1.00 | 48.56 | B |
| ATOM | 2885 | CG | LEU B | 494 | 79.698 | 4.594 | 54.604 | 1.00 | 48.11 | B |
| ATOM | 2886 | CD1 | LEU B | 494 | 78.587 | 3.650 | 55.032 | 1.00 | 47.88 | B |
| ATOM | 2887 | CD2 | LEU B | 494 | 79.156 | 5.988 | 54.313 | 1.00 | 49.20 | B |
| ATOM | 2888 | C | LEU B | 494 | 82.298 | 3.086 | 54.618 | 1.00 | 48.60 | B |
| ATOM | 2889 | O | LEU B | 494 | 83.009 | 3.938 | 54.096 | 1.00 | 48.84 | B |
| ATOM | 2890 | N | PRO B | 495 | 82.170 | 1.856 | 54.109 | 1.00 | 48.59 | B |
| ATOM | 2891 | CD | PRO B | 495 | 81.485 | 0.713 | 54.729 | 1.00 | 48.94 | B |
| ATOM | 2892 | CA | PRO B | 495 | 82.850 | 1.438 | 52.887 | 1.00 | 50.38 | B |
| ATOM | 2893 | CB | PRO B | 495 | 82.345 | 0.014 | 52.693 | 1.00 | 49.35 | B |
| ATOM | 2894 | CG | PRO B | 495 | 82.201 | −0.461 | 54.107 | 1.00 | 48.48 | B |
| ATOM | 2895 | C | PRO B | 495 | 82.561 | 2.342 | 51.693 | 1.00 | 52.37 | B |
| ATOM | 2896 | O | PRO B | 495 | 81.415 | 2.741 | 51.456 | 1.00 | 53.23 | B |
| ATOM | 2897 | N | LYS B | 496 | 83.625 | 2.669 | 50.963 | 1.00 | 53.80 | B |
| ATOM | 2898 | CA | LYS B | 496 | 83.557 | 3.519 | 49.784 | 1.00 | 54.63 | B |
| ATOM | 2899 | CB | LYS B | 496 | 84.934 | 4.155 | 49.529 | 1.00 | 55.28 | B |
| ATOM | 2900 | CG | LYS B | 496 | 86.118 | 3.196 | 49.670 | 1.00 | 54.90 | B |
| ATOM | 2901 | CD | LYS B | 496 | 87.443 | 3.945 | 49.624 | 1.00 | 54.86 | B |
| ATOM | 2902 | CE | LYS B | 496 | 88.625 | 3.017 | 49.840 | 0.00 | 55.42 | B |
| ATOM | 2903 | NZ | LYS B | 496 | 89.917 | 3.759 | 49.828 | 0.00 | 55.70 | B |
| ATOM | 2904 | C | LYS B | 496 | 83.100 | 2.731 | 48.559 | 1.00 | 54.70 | B |
| ATOM | 2905 | O | LYS B | 496 | 82.693 | 3.317 | 47.553 | 1.00 | 54.41 | B |
| ATOM | 2906 | N | ASP B | 508 | 80.076 | −10.950 | 44.143 | 1.00 | 87.67 | B |
| ATOM | 2907 | CA | ASP B | 508 | 81.314 | −10.630 | 44.843 | 1.00 | 87.56 | B |
| ATOM | 2908 | CB | ASP B | 508 | 82.398 | −10.224 | 43.855 | 1.00 | 85.57 | B |
| ATOM | 2909 | CG | ASP B | 508 | 82.370 | −8.742 | 43.565 | 1.00 | 84.40 | B |
| ATOM | 2910 | OD1 | ASP B | 508 | 81.334 | −8.260 | 43.072 | 1.00 | 83.24 | B |
| ATOM | 2911 | OD2 | ASP B | 508 | 83.371 | −8.056 | 43.855 | 1.00 | 83.59 | B |
| ATOM | 2912 | C | ASP B | 508 | 81.080 | −9.451 | 45.781 | 1.00 | 88.88 | B |
| ATOM | 2913 | O | ASP B | 508 | 82.036 | −8.810 | 46.225 | 1.00 | 89.19 | B |
| ATOM | 2914 | N | TYR B | 509 | 79.812 | −9.155 | 46.066 | 1.00 | 89.80 | B |
| ATOM | 2915 | CA | TYR B | 509 | 79.455 | −8.033 | 46.939 | 1.00 | 89.61 | B |
| ATOM | 2916 | CB | TYR B | 509 | 78.210 | −7.309 | 46.397 | 1.00 | 90.86 | B |
| ATOM | 2917 | CG | TYR B | 509 | 78.488 | −5.981 | 45.705 | 1.00 | 92.99 | B |
| ATOM | 2918 | CD1 | TYR B | 509 | 77.576 | −5.455 | 44.774 | 1.00 | 94.09 | B |
| ATOM | 2919 | CE1 | TYR B | 509 | 77.817 | −4.236 | 44.120 | 1.00 | 94.93 | B |
| ATOM | 2920 | CD2 | TYR B | 509 | 79.653 | −5.252 | 45.974 | 1.00 | 93.03 | B |
| ATOM | 2921 | CE2 | TYR B | 509 | 79.906 | −4.032 | 45.329 | 1.00 | 94.38 | B |
| ATOM | 2922 | CZ | TYR B | 509 | 78.986 | −3.530 | 44.401 | 1.00 | 95.32 | B |
| ATOM | 2923 | OH | TYR B | 509 | 79.241 | −2.342 | 43.743 | 1.00 | 95.61 | B |
| ATOM | 2924 | C | TYR B | 509 | 79.236 | −8.425 | 48.404 | 1.00 | 88.37 | B |
| ATOM | 2925 | O | TYR B | 509 | 79.772 | −7.770 | 49.301 | 1.00 | 89.80 | B |
| ATOM | 2926 | N | PRO B | 510 | 78.448 | −9.487 | 48.672 | 1.00 | 86.15 | B |
| ATOM | 2927 | CD | PRO B | 510 | 77.598 | −10.261 | 47.751 | 1.00 | 84.82 | B |
| ATOM | 2928 | CA | PRO B | 510 | 78.214 | −9.897 | 50.067 | 1.00 | 83.98 | B |
| ATOM | 2929 | CB | PRO B | 510 | 77.276 | −11.093 | 49.923 | 1.00 | 84.08 | B |
| ATOM | 2930 | CG | PRO B | 510 | 76.518 | −10.763 | 48.676 | 1.00 | 84.98 | B |
| ATOM | 2931 | C | PRO B | 510 | 79.508 | −10.265 | 50.800 | 1.00 | 81.81 | B |
| ATOM | 2932 | O | PRO B | 510 | 79.788 | −11.441 | 51.017 | 1.00 | 81.76 | B |
| ATOM | 2933 | N | LEU B | 511 | 80.289 | −9.254 | 51.176 | 1.00 | 79.52 | B |
| ATOM | 2934 | CA | LEU B | 511 | 81.556 | −9.462 | 51.875 | 1.00 | 77.45 | B |
| ATOM | 2935 | CB | LEU B | 511 | 82.466 | −8.246 | 51.701 | 1.00 | 75.86 | B |
| ATOM | 2936 | CG | LEU B | 511 | 82.803 | −7.866 | 50.264 | 1.00 | 74.15 | B |
| ATOM | 2937 | CD1 | LEU B | 511 | 83.709 | −6.653 | 50.229 | 1.00 | 72.84 | B |
| ATOM | 2938 | CD2 | LEU B | 511 | 83.463 | −9.053 | 49.601 | 1.00 | 74.63 | B |
| ATOM | 2939 | C | LEU B | 511 | 81.323 | −9.689 | 53.355 | 1.00 | 77.25 | B |
| ATOM | 2940 | O | LEU B | 511 | 80.682 | −8.878 | 54.020 | 1.00 | 77.95 | B |
| ATOM | 2941 | N | VAL B | 512 | 81.856 | −10.785 | 53.877 | 1.00 | 76.40 | B |
| ATOM | 2942 | CA | VAL B | 512 | 81.683 | −11.100 | 55.286 | 1.00 | 75.66 | B |
| ATOM | 2943 | CB | VAL B | 512 | 81.075 | −12.495 | 55.458 | 1.00 | 74.68 | B |
| ATOM | 2944 | CG1 | VAL B | 512 | 80.748 | −12.736 | 56.917 | 1.00 | 73.65 | B |
| ATOM | 2945 | CG2 | VAL B | 512 | 79.843 | −12.625 | 54.587 | 1.00 | 73.90 | B |
| ATOM | 2946 | C | VAL B | 512 | 83.009 | −11.040 | 56.029 | 1.00 | 75.90 | B |
| ATOM | 2947 | O | VAL B | 512 | 84.015 | −11.558 | 55.560 | 1.00 | 76.65 | B |
| ATOM | 2948 | N | PHE B | 513 | 83.007 | −10.394 | 57.187 | 1.00 | 75.87 | B |
| ATOM | 2949 | CA | PHE B | 513 | 84.218 | −10.275 | 57.986 | 1.00 | 75.67 | B |
| ATOM | 2950 | CB | PHE B | 513 | 84.669 | −8.820 | 58.058 | 1.00 | 74.40 | B |
| ATOM | 2951 | CG | PHE B | 513 | 84.758 | −8.148 | 56.722 | 1.00 | 74.06 | B |
| ATOM | 2952 | CD1 | PHE B | 513 | 83.611 | −7.885 | 55.981 | 1.00 | 74.40 | B |
| ATOM | 2953 | CD2 | PHE B | 513 | 85.987 | −7.765 | 56.208 | 1.00 | 74.00 | B |
| ATOM | 2954 | CE1 | PHE B | 513 | 83.686 | −7.246 | 54.752 | 1.00 | 74.32 | B |
| ATOM | 2955 | CE2 | PHE B | 513 | 86.077 | −7.126 | 54.979 | 1.00 | 73.93 | B |
| ATOM | 2956 | CZ | PHE B | 513 | 84.921 | −6.865 | 54.249 | 1.00 | 74.51 | B |
| ATOM | 2957 | C | PHE B | 513 | 83.893 | −10.780 | 59.378 | 1.00 | 76.17 | B |
| ATOM | 2958 | O | PHE B | 513 | 83.367 | −10.041 | 60.207 | 1.00 | 76.66 | B |
| ATOM | 2959 | N | HIS B | 514 | 84.203 | −12.043 | 59.631 | 1.00 | 76.08 | B |
| ATOM | 2960 | CA | HIS B | 514 | 83.906 | −12.636 | 60.919 | 1.00 | 76.38 | B |
| ATOM | 2961 | CB | HIS B | 514 | 83.697 | −14.139 | 60.758 | 1.00 | 78.44 | B |
| ATOM | 2962 | CG | HIS B | 514 | 84.803 | −14.820 | 60.022 | 1.00 | 81.60 | B |
| ATOM | 2963 | CD2 | HIS B | 514 | 85.579 | −15.876 | 60.360 | 1.00 | 82.68 | B |
| ATOM | 2964 | ND1 | HIS B | 514 | 85.232 | −14.407 | 58.780 | 1.00 | 82.91 | B |
| ATOM | 2965 | CE1 | HIS B | 514 | 86.228 | −15.180 | 58.384 | 1.00 | 84.06 | B |
| ATOM | 2966 | NE2 | HIS B | 514 | 86.458 | −16.079 | 59.324 | 1.00 | 83.92 | B |
| ATOM | 2967 | C | HIS B | 514 | 84.981 | −12.355 | 61.948 | 1.00 | 75.65 | B |
| ATOM | 2968 | O | HIS B | 514 | 84.997 | −12.964 | 63.016 | 1.00 | 75.17 | B |
| ATOM | 2969 | N | THR B | 515 | 85.880 | −11.431 | 61.632 | 1.00 | 75.12 | B |
| ATOM | 2970 | CA | THR B | 515 | 86.931 | −11.076 | 62.573 | 1.00 | 75.68 | B |
| ATOM | 2971 | CB | THR B | 515 | 88.219 | −11.871 | 62.315 | 1.00 | 75.87 | B |
| ATOM | 2972 | OG1 | THR B | 515 | 87.936 | −13.276 | 62.363 | 1.00 | 75.87 | B |
| ATOM | 2973 | CG2 | THR B | 515 | 89.255 | −11.538 | 63.375 | 1.00 | 74.95 | B |
| ATOM | 2974 | C | THR B | 515 | 87.245 | −9.590 | 62.492 | 1.00 | 75.55 | B |
| ATOM | 2975 | O | THR B | 515 | 87.642 | −9.087 | 61.445 | 1.00 | 75.60 | B |
| ATOM | 2976 | N | PRO B | 516 | 87.061 | −8.869 | 63.606 | 1.00 | 75.61 | B |
| ATOM | 2977 | CD | PRO B | 516 | 86.520 | −9.405 | 64.867 | 1.00 | 76.30 | B |
| ATOM | 2978 | CA | PRO B | 516 | 87.308 | −7.430 | 63.722 | 1.00 | 75.87 | B |
| ATOM | 2979 | CB | PRO B | 516 | 87.056 | −7.161 | 65.202 | 1.00 | 76.07 | B |
| ATOM | 2980 | CG | PRO B | 516 | 86.008 | −8.162 | 65.550 | 1.00 | 76.54 | B |
| ATOM | 2981 | C | PRO B | 516 | 88.719 | −7.025 | 63.299 | 1.00 | 76.37 | B |
| ATOM | 2982 | O | PRO B | 516 | 88.897 | −6.183 | 62.415 | 1.00 | 75.84 | B |
| ATOM | 2983 | N | GLU B | 517 | 89.714 | −7.626 | 63.949 | 1.00 | 76.93 | B |
| ATOM | 2984 | CA | GLU B | 517 | 91.126 | −7.356 | 63.676 | 1.00 | 76.76 | B |
| ATOM | 2985 | CB | GLU B | 517 | 91.996 | −8.320 | 64.492 | 1.00 | 78.63 | B |
| ATOM | 2986 | CG | GLU B | 517 | 93.496 | −8.083 | 64.384 | 1.00 | 81.28 | B |
| ATOM | 2987 | CD | GLU B | 517 | 93.944 | −6.839 | 65.123 | 1.00 | 82.27 | B |
| ATOM | 2988 | OE1 | GLU B | 517 | 95.171 | −6.593 | 65.192 | 1.00 | 83.01 | B |
| ATOM | 2989 | OE2 | GLU B | 517 | 93.066 | −6.107 | 65.630 | 1.00 | 82.62 | B |
| ATOM | 2990 | C | GLU B | 517 | 91.448 | −7.505 | 62.189 | 1.00 | 75.46 | B |
| ATOM | 2991 | O | GLU B | 517 | 92.318 | −6.812 | 61.663 | 1.00 | 75.08 | B |
| ATOM | 2992 | N | GLU B | 518 | 90.733 | −8.411 | 61.525 | 1.00 | 73.54 | B |
| ATOM | 2993 | CA | GLU B | 518 | 90.927 | −8.681 | 60.106 | 1.00 | 71.48 | B |
| ATOM | 2994 | CB | GLU B | 518 | 90.530 | −10.124 | 59.793 | 0.00 | 72.44 | B |
| ATOM | 2995 | CG | GLU B | 518 | 90.797 | −10.526 | 58.359 | 0.00 | 73.10 | B |
| ATOM | 2996 | CD | GLU B | 518 | 90.441 | −11.970 | 58.079 | 0.00 | 73.51 | B |
| ATOM | 2997 | OE1 | GLU B | 518 | 89.258 | −12.335 | 58.243 | 0.00 | 73.72 | B |
| ATOM | 2998 | OE2 | GLU B | 518 | 91.346 | −12.741 | 57.695 | 0.00 | 73.72 | B |
| ATOM | 2999 | C | GLU B | 518 | 90.145 | −7.730 | 59.199 | 1.00 | 70.28 | B |
| ATOM | 3000 | O | GLU B | 518 | 90.596 | −7.400 | 58.098 | 1.00 | 69.72 | B |
| ATOM | 3001 | N | ASN B | 519 | 88.977 | −7.296 | 59.676 | 1.00 | 68.04 | B |
| ATOM | 3002 | CA | ASN B | 519 | 88.087 | −6.380 | 58.947 | 1.00 | 65.05 | B |
| ATOM | 3003 | CB | ASN B | 519 | 86.773 | −6.224 | 59.718 | 1.00 | 63.79 | B |
| ATOM | 3004 | CG | ASN B | 519 | 85.805 | −5.267 | 59.053 | 1.00 | 62.26 | B |
| ATOM | 3005 | OD1 | ASN B | 519 | 84.702 | −5.066 | 59.548 | 1.00 | 62.79 | B |
| ATOM | 3006 | ND2 | ASN B | 519 | 86.204 | −4.676 | 57.933 | 1.00 | 60.05 | B |
| ATOM | 3007 | C | ASN B | 519 | 88.716 | −5.000 | 58.728 | 1.00 | 63.93 | B |
| ATOM | 3008 | O | ASN B | 519 | 88.842 | −4.220 | 59.669 | 1.00 | 64.23 | B |
| ATOM | 3009 | N | PRO B | 520 | 89.090 | −4.673 | 57.473 | 1.00 | 62.40 | B |
| ATOM | 3010 | CD | PRO B | 520 | 88.866 | −5.499 | 56.274 | 1.00 | 61.90 | B |
| ATOM | 3011 | CA | PRO B | 520 | 89.711 | −3.396 | 57.096 | 1.00 | 60.68 | B |
| ATOM | 3012 | CB | PRO B | 520 | 89.820 | −3.497 | 55.577 | 1.00 | 60.59 | B |
| ATOM | 3013 | CG | PRO B | 520 | 89.895 | −4.957 | 55.331 | 1.00 | 61.94 | B |
| ATOM | 3014 | C | PRO B | 520 | 88.906 | −2.174 | 57.500 | 1.00 | 60.35 | B |
| ATOM | 3015 | O | PRO B | 520 | 89.403 | −1.049 | 57.445 | 1.00 | 60.91 | B |
| ATOM | 3016 | N | TYR B | 521 | 87.657 | −2.399 | 57.888 | 1.00 | 58.53 | B |
| ATOM | 3017 | CA | TYR B | 521 | 86.774 | −1.314 | 58.276 | 1.00 | 56.62 | B |
| ATOM | 3018 | CB | TYR B | 521 | 85.410 | −1.538 | 57.636 | 1.00 | 56.52 | B |
| ATOM | 3019 | CG | TYR B | 521 | 85.470 | −1.631 | 56.133 | 1.00 | 55.43 | B |
| ATOM | 3020 | CD1 | TYR B | 521 | 85.625 | −0.493 | 55.356 | 1.00 | 53.35 | B |
| ATOM | 3021 | CE1 | TYR B | 521 | 85.684 | −0.568 | 53.980 | 1.00 | 52.25 | B |
| ATOM | 3022 | CD2 | TYR B | 521 | 85.378 | −2.860 | 55.487 | 1.00 | 55.47 | B |
| ATOM | 3023 | CE2 | TYR B | 521 | 85.437 | −2.946 | 54.109 | 1.00 | 54.04 | B |
| ATOM | 3024 | CZ | TYR B | 521 | 85.587 | −1.792 | 53.360 | 1.00 | 53.20 | B |
| ATOM | 3025 | OH | TYR B | 521 | 85.611 | −1.862 | 51.986 | 1.00 | 53.19 | B |
| ATOM | 3026 | C | TYR B | 521 | 86.618 | −1.168 | 59.782 | 1.00 | 56.07 | B |
| ATOM | 3027 | O | TYR B | 521 | 86.225 | −0.109 | 60.264 | 1.00 | 55.68 | B |
| ATOM | 3028 | N | PHE B | 522 | 86.929 | −2.227 | 60.521 | 1.00 | 55.73 | B |
| ATOM | 3029 | CA | PHE B | 522 | 86.797 | −2.207 | 61.969 | 1.00 | 55.79 | B |
| ATOM | 3030 | CB | PHE B | 522 | 86.993 | −3.605 | 62.543 | 1.00 | 57.01 | B |
| ATOM | 3031 | CG | PHE B | 522 | 86.935 | −3.654 | 64.046 | 1.00 | 59.45 | B |
| ATOM | 3032 | CD1 | PHE B | 522 | 85.730 | −3.458 | 64.719 | 1.00 | 60.43 | B |
| ATOM | 3033 | CD2 | PHE B | 522 | 88.084 | −3.895 | 64.794 | 1.00 | 59.23 | B |
| ATOM | 3034 | CE1 | PHE B | 522 | 85.672 | −3.505 | 66.119 | 1.00 | 60.50 | B |
| ATOM | 3035 | CE2 | PHE B | 522 | 88.032 | −3.943 | 66.192 | 1.00 | 59.21 | B |
| ATOM | 3036 | CZ | PHE B | 522 | 86.825 | −3.749 | 66.856 | 1.00 | 58.74 | B |
| ATOM | 3037 | C | PHE B | 522 | 87.764 | −1.267 | 62.668 | 1.00 | 55.79 | B |
| ATOM | 3038 | O | PHE B | 522 | 88.899 | −1.643 | 62.962 | 1.00 | 56.44 | B |
| ATOM | 3039 | N | VAL B | 523 | 87.314 | −0.048 | 62.951 | 1.00 | 56.06 | B |
| ATOM | 3040 | CA | VAL B | 523 | 88.155 | 0.924 | 63.650 | 1.00 | 55.15 | B |
| ATOM | 3041 | CB | VAL B | 523 | 87.445 | 2.271 | 63.817 | 1.00 | 53.14 | B |
| ATOM | 3042 | CG1 | VAL B | 523 | 88.324 | 3.223 | 64.601 | 1.00 | 51.48 | B |
| ATOM | 3043 | CG2 | VAL B | 523 | 87.113 | 2.844 | 62.460 | 1.00 | 53.68 | B |
| ATOM | 3044 | C | VAL B | 523 | 88.506 | 0.384 | 65.035 | 1.00 | 55.15 | B |
| ATOM | 3045 | O | VAL B | 523 | 89.675 | 0.140 | 65.331 | 1.00 | 55.89 | B |
| ATOM | 3046 | N | GLY B | 524 | 87.489 | 0.187 | 65.870 | 1.00 | 53.67 | B |
| ATOM | 3047 | CA | GLY B | 524 | 87.726 | −0.325 | 67.205 | 1.00 | 52.15 | B |
| ATOM | 3048 | C | GLY B | 524 | 86.521 | −0.286 | 68.127 | 1.00 | 50.48 | B |
| ATOM | 3049 | O | GLY B | 524 | 85.504 | 0.329 | 67.821 | 1.00 | 50.52 | B |
| ATOM | 3050 | N | VAL B | 525 | 86.643 | −0.933 | 69.277 | 1.00 | 48.79 | B |
| ATOM | 3051 | CA | VAL B | 525 | 85.555 | −0.975 | 70.232 | 1.00 | 48.07 | B |
| ATOM | 3052 | CB | VAL B | 525 | 85.354 | −2.418 | 70.744 | 1.00 | 46.89 | B |
| ATOM | 3053 | CG1 | VAL B | 525 | 86.674 | −3.138 | 70.750 | 1.00 | 45.99 | B |
| ATOM | 3054 | CG2 | VAL B | 525 | 84.732 | −2.403 | 72.130 | 1.00 | 45.90 | B |
| ATOM | 3055 | C | VAL B | 525 | 85.800 | −0.015 | 71.392 | 1.00 | 47.57 | B |
| ATOM | 3056 | O | VAL B | 525 | 86.938 | 0.175 | 71.805 | 1.00 | 46.79 | B |
| ATOM | 3057 | N | LEU B | 526 | 84.727 | 0.603 | 71.894 | 1.00 | 48.52 | B |
| ATOM | 3058 | CA | LEU B | 526 | 84.811 | 1.567 | 73.005 | 1.00 | 48.97 | B |
| ATOM | 3059 | CB | LEU B | 526 | 84.199 | 2.910 | 72.583 | 1.00 | 47.57 | B |
| ATOM | 3060 | CG | LEU B | 526 | 84.696 | 3.601 | 71.310 | 1.00 | 47.85 | B |
| ATOM | 3061 | CD1 | LEU B | 526 | 83.713 | 4.694 | 70.936 | 1.00 | 48.90 | B |
| ATOM | 3062 | CD2 | LEU B | 526 | 86.082 | 4.170 | 71.496 | 1.00 | 46.34 | B |
| ATOM | 3063 | C | LEU B | 526 | 84.078 | 1.047 | 74.257 | 1.00 | 49.07 | B |
| ATOM | 3064 | O | LEU B | 526 | 82.862 | 1.207 | 74.382 | 1.00 | 49.55 | B |
| ATOM | 3065 | N | ARG B | 527 | 84.820 | 0.425 | 75.174 | 1.00 | 48.77 | B |
| ATOM | 3066 | CA | ARG B | 527 | 84.239 | −0.119 | 76.402 | 1.00 | 48.18 | B |
| ATOM | 3067 | CB | ARG B | 527 | 85.016 | −1.349 | 76.877 | 1.00 | 47.43 | B |
| ATOM | 3068 | CG | ARG B | 527 | 84.725 | −2.604 | 76.074 | 1.00 | 47.62 | B |
| ATOM | 3069 | CD | ARG B | 527 | 85.540 | −3.794 | 76.557 | 1.00 | 45.88 | B |
| ATOM | 3070 | NE | ARG B | 527 | 86.528 | −4.196 | 75.561 | 1.00 | 44.94 | B |
| ATOM | 3071 | CZ | ARG B | 527 | 86.259 | −4.860 | 74.439 | 1.00 | 43.61 | B |
| ATOM | 3072 | NH1 | ARG B | 527 | 85.014 | −5.218 | 74.152 | 1.00 | 41.64 | B |
| ATOM | 3073 | NH2 | ARG B | 527 | 87.246 | −5.158 | 73.594 | 1.00 | 41.55 | B |
| ATOM | 3074 | C | ARG B | 527 | 84.214 | 0.907 | 77.515 | 1.00 | 47.73 | B |
| ATOM | 3075 | O | ARG B | 527 | 85.162 | 1.662 | 77.703 | 1.00 | 46.73 | B |
| ATOM | 3076 | N | GLY B | 528 | 83.120 | 0.929 | 78.259 | 1.00 | 48.05 | B |
| ATOM | 3077 | CA | GLY B | 528 | 83.013 | 1.888 | 79.338 | 1.00 | 49.61 | B |
| ATOM | 3078 | C | GLY B | 528 | 81.677 | 1.823 | 80.047 | 1.00 | 50.20 | B |
| ATOM | 3079 | O | GLY B | 528 | 81.510 | 2.397 | 81.120 | 1.00 | 50.44 | B |
| ATOM | 3080 | N | HIS B | 529 | 80.718 | 1.126 | 79.448 | 1.00 | 51.40 | B |
| ATOM | 3081 | CA | HIS B | 529 | 79.392 | 0.990 | 80.040 | 1.00 | 52.69 | B |
| ATOM | 3082 | CB | HIS B | 529 | 78.308 | 1.270 | 78.997 | 1.00 | 53.42 | B |
| ATOM | 3083 | CG | HIS B | 529 | 77.908 | 2.711 | 78.931 | 1.00 | 54.66 | B |
| ATOM | 3084 | CD2 | HIS B | 529 | 78.036 | 3.710 | 79.836 | 1.00 | 54.96 | B |
| ATOM | 3085 | ND1 | HIS B | 529 | 77.284 | 3.264 | 77.835 | 1.00 | 54.53 | B |
| ATOM | 3086 | CE1 | HIS B | 529 | 77.046 | 4.542 | 78.066 | 1.00 | 54.69 | B |
| ATOM | 3087 | NE2 | HIS B | 529 | 77.493 | 4.837 | 79.273 | 1.00 | 55.98 | B |
| ATOM | 3088 | C | HIS B | 529 | 79.174 | −0.376 | 80.664 | 1.00 | 52.47 | B |
| ATOM | 3089 | O | HIS B | 529 | 79.576 | −1.396 | 80.112 | 1.00 | 53.72 | B |
| ATOM | 3090 | N | MSE B | 530 | 78.531 | −0.379 | 81.825 | 1.00 | 52.35 | B |
| ATOM | 3091 | CA | MSE B | 530 | 78.260 | −1.603 | 82.568 | 1.00 | 51.79 | B |
| ATOM | 3092 | CB | MSE B | 530 | 78.325 | −1.325 | 84.068 | 1.00 | 54.89 | B |
| ATOM | 3093 | CG | MSE B | 530 | 79.640 | −0.727 | 84.520 | 1.00 | 59.05 | B |
| ATOM | 3094 | SE | MSE B | 530 | 81.160 | −1.831 | 84.066 | 1.00 | 66.13 | B |
| ATOM | 3095 | CE | MSE B | 530 | 81.008 | −3.077 | 85.560 | 1.00 | 62.20 | B |
| ATOM | 3096 | C | MSE B | 530 | 76.894 | −2.153 | 82.241 | 1.00 | 50.13 | B |
| ATOM | 3097 | O | MSE B | 530 | 76.318 | −2.887 | 83.036 | 1.00 | 49.11 | B |
| ATOM | 3098 | N | ALA B | 531 | 76.379 | −1.800 | 81.070 | 1.00 | 49.94 | B |
| ATOM | 3099 | CA | ALA B | 531 | 75.055 | −2.246 | 80.658 | 1.00 | 48.69 | B |
| ATOM | 3100 | CB | ALA B | 531 | 73.999 | −1.553 | 81.498 | 1.00 | 48.43 | B |
| ATOM | 3101 | C | ALA B | 531 | 74.843 | −1.928 | 79.189 | 1.00 | 47.77 | B |
| ATOM | 3102 | O | ALA B | 531 | 75.669 | −1.256 | 78.566 | 1.00 | 48.05 | B |
| ATOM | 3103 | N | SER B | 532 | 73.731 | −2.414 | 78.645 | 1.00 | 46.82 | B. |
| ATOM | 3104 | CA | SER B | 532 | 73.380 | −2.196 | 77.242 | 1.00 | 45.70 | B |
| ATOM | 3105 | CB | SER B | 532 | 72.036 | −2.855 | 76.921 | 1.00 | 45.79 | B |
| ATOM | 3106 | OG | SER B | 532 | 71.413 | −2.223 | 75.811 | 1.00 | 44.70 | B |
| ATOM | 3107 | C | SER B | 532 | 73.306 | −0.733 | 76.839 | 1.00 | 44.93 | B |
| ATOM | 3108 | O | SER B | 532 | 72.834 | 0.118 | 77.595 | 1.00 | 45.32 | B |
| ATOM | 3109 | N | VAL B | 533 | 73.775 | −0.469 | 75.627 | 1.00 | 43.79 | B |
| ATOM | 3110 | CA | VAL B | 533 | 73.771 | 0.865 | 75.038 | 1.00 | 43.92 | B |
| ATOM | 3111 | CB | VAL B | 533 | 75.002 | 1.045 | 74.123 | 1.00 | 42.68 | B |
| ATOM | 3112 | CG1 | VAL B | 533 | 74.893 | 2.335 | 73.336 | 1.00 | 42.48 | B |
| ATOM | 3113 | CG2 | VAL B | 533 | 76.274 | 1.027 | 74.965 | 1.00 | 41.21 | B |
| ATOM | 3114 | C | VAL B | 533 | 72.480 | 1.013 | 74.215 | 1.00 | 43.88 | B |
| ATOM | 3115 | O | VAL B | 533 | 72.398 | 0.518 | 73.085 | 1.00 | 43.85 | B |
| ATOM | 3116 | N | ARG B | 534 | 71.479 | 1.689 | 74.787 | 1.00 | 42.61 | B |
| ATOM | 3117 | CA | ARG B | 534 | 70.174 | 1.867 | 74.136 | 1.00 | 40.98 | B |
| ATOM | 3118 | CB | ARG B | 534 | 69.074 | 2.081 | 75.177 | 1.00 | 41.23 | B |
| ATOM | 3119 | CG | ARG B | 534 | 67.896 | 1.146 | 75.017 | 1.00 | 42.21 | B |
| ATOM | 3120 | CD | ARG B | 534 | 66.586 | 1.862 | 74.752 | 1.00 | 43.49 | B |
| ATOM | 3121 | NE | ARG B | 534 | 66.301 | 2.077 | 73.331 | 1.00 | 45.72 | B |
| ATOM | 3122 | CZ | ARG B | 534 | 65.097 | 1.903 | 72.792 | 1.00 | 47.54 | B |
| ATOM | 3123 | NH1 | ARG B | 534 | 64.093 | 1.508 | 73.563 | 1.00 | 46.82 | B |
| ATOM | 3124 | NH2 | ARG B | 534 | 64.884 | 2.136 | 71.499 | 1.00 | 50.38 | B |
| ATOM | 3125 | C | ARG B | 534 | 70.104 | 3.015 | 73.162 | 1.00 | 39.36 | B |
| ATOM | 3126 | O | ARG B | 534 | 69.339 | 2.975 | 72.200 | 1.00 | 38.05 | B |
| ATOM | 3127 | N | THR B | 535 | 70.889 | 4.050 | 73.426 | 1.00 | 38.74 | B |
| ATOM | 3128 | CA | THR B | 535 | 70.869 | 5.217 | 72.566 | 1.00 | 38.25 | B |
| ATOM | 3129 | CB | THR B | 535 | 70.062 | 6.362 | 73.233 | 1.00 | 39.86 | B |
| ATOM | 3130 | OG1 | THR B | 535 | 70.130 | 7.534 | 72.411 | 1.00 | 42.15 | B |
| ATOM | 3131 | CG2 | THR B | 535 | 70.596 | 6.661 | 74.630 | 1.00 | 39.79 | B |
| ATOM | 3132 | C | THR B | 535 | 72.263 | 5.686 | 72.189 | 1.00 | 36.40 | B |
| ATOM | 3133 | O | THR B | 535 | 73.212 | 5.478 | 72.934 | 1.00 | 34.48 | B |
| ATOM | 3134 | N | VAL B | 536 | 72.369 | 6.300 | 71.013 | 1.00 | 36.97 | B |
| ATOM | 3135 | CA | VAL B | 536 | 73.637 | 6.798 | 70.482 | 1.00 | 38.48 | B |
| ATOM | 3136 | CB | VAL B | 536 | 74.365 | 5.725 | 69.663 | 1.00 | 39.79 | B |
| ATOM | 3137 | CG1 | VAL B | 536 | 75.570 | 6.344 | 68.958 | 1.00 | 39.98 | B |
| ATOM | 3138 | CG2 | VAL B | 536 | 74.807 | 4.588 | 70.561 | 1.00 | 42.70 | B |
| ATOM | 3139 | C | VAL B | 536 | 73.417 | 7.969 | 69.538 | 1.00 | 37.98 | B |
| ATOM | 3140 | O | VAL B | 536 | 72.705 | 7.836 | 68.544 | 1.00 | 39.94 | B |
| ATOM | 3141 | N | SER B | 537 | 74.037 | 9.105 | 69.832 | 1.00 | 36.75 | B |
| ATOM | 3142 | CA | SER B | 537 | 73.904 | 10.281 | 68.978 | 1.00 | 35.47 | B |
| ATOM | 3143 | CB | SER B | 537 | 72.974 | 11.296 | 69.646 | 1.00 | 36.17 | B |
| ATOM | 3144 | OG | SER B | 537 | 73.057 | 12.572 | 69.032 | 1.00 | 36.43 | B |
| ATOM | 3145 | C | SER B | 537 | 75.280 | 10.889 | 68.744 | 1.00 | 34.85 | B |
| ATOM | 3146 | O | SER B | 537 | 76.071 | 11.009 | 69.671 | 1.00 | 33.64 | B |
| ATOM | 3147 | N | GLY B | 538 | 75.575 | 11.258 | 67.507 | 1.00 | 34.65 | B |
| ATOM | 3148 | CA | GLY B | 538 | 76.875 | 11.836 | 67.243 | 1.00 | 37.80 | B |
| ATOM | 3149 | C | GLY B | 538 | 76.900 | 12.740 | 66.030 | 1.00 | 39.65 | B |
| ATOM | 3150 | O | GLY B | 538 | 75.937 | 12.783 | 65.270 | 1.00 | 39.10 | B |
| ATOM | 3151 | N | HIS B | 539 | 78.004 | 13.462 | 65.857 | 1.00 | 42.51 | B |
| ATOM | 3152 | CA | HIS B | 539 | 78.193 | 14.382 | 64.736 | 1.00 | 44.95 | B |
| ATOM | 3153 | CB | HIS B | 539 | 77.370 | 15.657 | 64.927 | 1.00 | 48.76 | B |
| ATOM | 3154 | CG | HIS B | 539 | 75.912 | 15.402 | 65.134 | 1.00 | 54.64 | B |
| ATOM | 3155 | CD2 | HIS B | 539 | 75.121 | 15.566 | 66.221 | 1.00 | 55.81 | B |
| ATOM | 3156 | ND1 | HIS B | 539 | 75.112 | 14.845 | 64.158 | 1.00 | 55.89 | B |
| ATOM | 3157 | CE1 | HIS B | 539 | 73.891 | 14.677 | 64.635 | 1.00 | 56.19 | B |
| ATOM | 3158 | NE2 | HIS B | 539 | 73.870 | 15.106 | 65.885 | 1.00 | 56.92 | B |
| ATOM | 3159 | C | HIS B | 539 | 79.665 | 14.753 | 64.709 | 1.00 | 45.08 | B |
| ATOM | 3160 | O | HIS B | 539 | 80.167 | 15.351 | 65.659 | 1.00 | 45.61 | B |
| ATOM | 3161 | N | GLY B | 540 | 80.357 | 14.409 | 63.627 | 1.00 | 44.60 | B |
| ATOM | 3162 | CA | GLY B | 540 | 81.773 | 14.727 | 63.538 | 1.00 | 44.51 | B |
| ATOM | 3163 | C | GLY B | 540 | 82.612 | 13.714 | 64.300 | 1.00 | 44.45 | B |
| ATOM | 3164 | O | GLY B | 540 | 82.356 | 12.519 | 64.188 | 1.00 | 44.43 | B |
| ATOM | 3165 | N | ASN B | 541 | 83.612 | 14.182 | 65.054 | 1.00 | 44.24 | B |
| ATOM | 3166 | CA | ASN B | 541 | 84.482 | 13.304 | 65.841 | 1.00 | 43.77 | B |
| ATOM | 3167 | CB | ASN B | 541 | 85.914 | 13.855 | 65.925 | 1.00 | 43.91 | B |
| ATOM | 3168 | CG | ASN B | 541 | 86.011 | 15.119 | 66.773 | 1.00 | 45.62 | B |
| ATOM | 3169 | OD1 | ASN B | 541 | 85.399 | 16.139 | 66.449 | 1.00 | 46.42 | B |
| ATOM | 3170 | ND2 | ASN B | 541 | 86.783 | 15.054 | 67.867 | 1.00 | 45.00 | B |
| ATOM | 3171 | C | ASN B | 541 | 83.949 | 13.134 | 67.257 | 1.00 | 43.38 | B |
| ATOM | 3172 | O | ASN B | 541 | 84.662 | 12.651 | 68.139 | 1.00 | 44.20 | B |
| ATOM | 3173 | N | ILE B | 542 | 82.697 | 13.530 | 67.475 | 1.00 | 41.65 | B |
| ATOM | 3174 | CA | ILE B | 542 | 82.067 | 13.418 | 68.789 | 1.00 | 40.51 | B |
| ATOM | 3175 | CB | ILE B | 542 | 81.681 | 14.821 | 69.350 | 1.00 | 41.10 | B |
| ATOM | 3176 | CG2 | ILE B | 542 | 81.158 | 14.690 | 70.779 | 1.00 | 39.51 | B |
| ATOM | 3177 | CG1 | ILE B | 542 | 82.893 | 15.753 | 69.325 | 1.00 | 39.00 | B |
| ATOM | 3178 | CD1 | ILE B | 542 | 84.043 | 15.278 | 70.192 | 1.00 | 38.09 | B |
| ATOM | 3179 | C | ILE B | 542 | 80.795 | 12.560 | 68.744 | 1.00 | 39.78 | B |
| ATOM | 3180 | O | ILE B | 542 | 79.939 | 12.740 | 67.870 | 1.00 | 38.52 | B |
| ATOM | 3181 | N | VAL B | 543 | 80.687 | 11.630 | 69.691 | 1.00 | 38.75 | B |
| ATOM | 3182 | CA | VAL B | 543 | 79.523 | 10.752 | 69.799 | 1.00 | 38.25 | B |
| ATOM | 3183 | CB | VAL B | 543 | 79.775 | 9.342 | 69.183 | 1.00 | 37.58 | B |
| ATOM | 3184 | CG1 | VAL B | 543 | 78.596 | 8.430 | 69.454 | 1.00 | 34.60 | B |
| ATOM | 3185 | CG2 | VAL B | 543 | 79.995 | 9.459 | 67.678 | 1.00 | 39.64 | B |
| ATOM | 3186 | C | VAL B | 543 | 79.198 | 10.573 | 71.270 | 1.00 | 37.91 | B |
| ATOM | 3187 | O | VAL B | 543 | 80.098 | 10.411 | 72.091 | 1.00 | 40.30 | B |
| ATOM | 3188 | N | VAL B | 544 | 77.913 | 10.602 | 71.605 | 1.00 | 35.82 | B |
| ATOM | 3189 | CA | VAL B | 544 | 77.490 | 10.435 | 72.985 | 1.00 | 32.38 | B |
| ATOM | 3190 | CB | VAL B | 544 | 76.637 | 11.600 | 73.437 | 1.00 | 31.34 | B |
| ATOM | 3191 | CG1 | VAL B | 544 | 76.364 | 11.471 | 74.913 | 1.00 | 31.93 | B |
| ATOM | 3192 | CG2 | VAL B | 544 | 77.321 | 12.904 | 73.104 | 1.00 | 30.92 | B |
| ATOM | 3193 | C | VAL B | 544 | 76.652 | 9.186 | 73.089 | 1.00 | 31.39 | B |
| ATOM | 3194 | O | VAL B | 544 | 75.764 | 8.976 | 72.279 | 1.00 | 32.39 | B |
| ATOM | 3195 | N | SER B | 545 | 76.913 | 8.359 | 74.089 | 1.00 | 31.37 | B |
| ATOM | 3196 | CA | SER B | 545 | 76.136 | 7.135 | 74.238 | 1.00 | 32.82 | B |
| ATOM | 3197 | CB | SER B | 545 | 77.067 | 5.911 | 74.166 | 1.00 | 33.04 | B |
| ATOM | 3198 | OG | SER B | 545 | 77.887 | 5.768 | 75.328 | 1.00 | 31.94 | B |
| ATOM | 3199 | C | SER B | 545 | 75.290 | 7.062 | 75.522 | 1.00 | 34.31 | B |
| ATOM | 3200 | O | SER B | 545 | 75.663 | 7.595 | 76.576 | 1.00 | 35.23 | B |
| ATOM | 3201 | N | GLY B | 546 | 74.157 | 6.373 | 75.421 | 1.00 | 35.10 | B |
| ATOM | 3202 | CA | GLY B | 546 | 73.271 | 6.209 | 76.558 | 1.00 | 35.16 | B |
| ATOM | 3203 | C | GLY B | 546 | 73.064 | 4.744 | 76.889 | 1.00 | 35.12 | B |
| ATOM | 3204 | O | GLY B | 546 | 72.627 | 3.952 | 76.048 | 1.00 | 36.06 | B |
| ATOM | 3205 | N | SER B | 547 | 73.370 | 4.377 | 78.122 | 1.00 | 34.92 | B |
| ATOM | 3206 | CA | SER B | 547 | 73.219 | 2.996 | 78.520 | 1.00 | 35.83 | B |
| ATOM | 3207 | CB | SER B | 547 | 74.542 | 2.450 | 79.054 | 1.00 | 36.42 | B |
| ATOM | 3208 | OG | SER B | 547 | 74.376 | 1.145 | 79.580 | 1.00 | 37.41 | B |
| ATOM | 3209 | C | SER B | 547 | 72.146 | 2.811 | 79.569 | 1.00 | 36.27 | B |
| ATOM | 3210 | O | SER B | 547 | 71.561 | 3.776 | 80.043 | 1.00 | 35.05 | B |
| ATOM | 3211 | N | TYR B | 548 | 71.881 | 1.555 | 79.908 | 1.00 | 38.32 | B |
| ATOM | 3212 | CA | TYR B | 548 | 70.890 | 1.227 | 80.916 | 1.00 | 40.30 | B |
| ATOM | 3213 | CB | TYR B | 548 | 70.288 | −0.146 | 80.629 | 1.00 | 42.68 | B |
| ATOM | 3214 | CG | TYR B | 548 | 69.117 | −0.134 | 79.670 | 1.00 | 45.80 | B |
| ATOM | 3215 | CD1 | TYR B | 548 | 68.593 | 1.068 | 79.174 | 1.00 | 47.24 | B |
| ATOM | 3216 | CE1 | TYR B | 548 | 67.472 | 1.074 | 78.345 | 1.00 | 47.96 | B |
| ATOM | 3217 | CD2 | TYR B | 548 | 68.494 | −1.322 | 79.302 | 1.00 | 45.36 | B |
| ATOM | 3218 | CE2 | TYR B | 548 | 67.385 | −1.325 | 78.481 | 1.00 | 46.33 | B |
| ATOM | 3219 | CZ | TYR B | 548 | 66.873 | −0.135 | 78.008 | 1.00 | 47.54 | B |
| ATOM | 3220 | OH | TYR B | 548 | 65.744 | −0.162 | 77.223 | 1.00 | 49.19 | B |
| ATOM | 3221 | C | TYR B | 548 | 71.550 | 1.228 | 82.289 | 1.00 | 40.55 | B |
| ATOM | 3222 | O | TYR B | 548 | 70.898 | 0.990 | 83.305 | 1.00 | 40.82 | B |
| ATOM | 3223 | N | ASP B | 549 | 72.852 | 1.496 | 82.315 | 1.00 | 40.33 | B |
| ATOM | 3224 | CA | ASP B | 549 | 73.574 | 1.523 | 83.572 | 1.00 | 40.21 | B |
| ATOM | 3225 | CB | ASP B | 549 | 75.053 | 1.177 | 83.352 | 1.00 | 38.64 | B |
| ATOM | 3226 | CG | ASP B | 549 | 75.813 | 2.287 | 82.659 | 1.00 | 39.21 | B |
| ATOM | 3227 | OD1 | ASP B | 549 | 75.195 | 3.348 | 82.399 | 1.00 | 39.42 | B |
| ATOM | 3228 | OD2 | ASP B | 549 | 77.025 | 2.106 | 82.389 | 1.00 | 35.60 | B |
| ATOM | 3229 | C | ASP B | 549 | 73.437 | 2.905 | 84.207 | 1.00 | 41.08 | B |
| ATOM | 3230 | O | ASP B | 549 | 74.180 | 3.251 | 85.125 | 1.00 | 41.91 | B |
| ATOM | 3231 | N | ASN B | 550 | 72.490 | 3.693 | 83.696 | 1.00 | 41.46 | B |
| ATOM | 3232 | CA | ASN B | 550 | 72.213 | 5.043 | 84.208 | 1.00 | 41.90 | B |
| ATOM | 3233 | CB | ASN B | 550 | 71.853 | 4.982 | 85.696 | 1.00 | 42.29 | B |
| ATOM | 3234 | CG | ASN B | 550 | 70.885 | 3.882 | 86.013 | 1.00 | 42.40 | B |
| ATOM | 3235 | OD1 | ASN B | 550 | 70.421 | 3.181 | 85.117 | 1.00 | 44.98 | B |
| ATOM | 3236 | ND2 | ASN B | 550 | 70.563 | 3.723 | 87.289 | 1.00 | 41.54 | B |
| ATOM | 3237 | C | ASN B | 550 | 73.430 | 5.944 | 84.049 | 1.00 | 41.35 | B |
| ATOM | 3238 | O | ASN B | 550 | 73.934 | 6.498 | 85.034 | 1.00 | 41.70 | B |
| ATOM | 3239 | N | THR B | 551 | 73.891 | 6.101 | 82.816 | 1.00 | 40.29 | B |
| ATOM | 3240 | CA | THR B | 551 | 75.081 | 6.890 | 82.562 | 1.00 | 40.46 | B |
| ATOM | 3241 | CB | THR B | 551 | 76.344 | 6.109 | 83.031 | 1.00 | 40.37 | B |
| ATOM | 3242 | OG1 | THR B | 551 | 76.524 | 6.278 | 84.437 | 1.00 | 40.39 | B |
| ATOM | 3243 | CG2 | THR B | 551 | 77.576 | 6.547 | 82.288 | 1.00 | 39.19 | B |
| ATOM | 3244 | C | THR B | 551 | 75.230 | 7.167 | 81.080 | 1.00 | 41.05 | B |
| ATOM | 3245 | O | THR B | 551 | 74.793 | 6.374 | 80.250 | 1.00 | 41.68 | B |
| ATOM | 3246 | N | LEU B | 552 | 75.849 | 8.295 | 80.754 | 1.00 | 40.71 | B |
| ATOM | 3247 | CA | LEU B | 552 | 76.098 | 8.659 | 79.368 | 1.00 | 40.16 | B |
| ATOM | 3248 | CB | LEU B | 552 | 75.354 | 9.943 | 78.994 | 1.00 | 39.03 | B |
| ATOM | 3249 | CG | LEU B | 552 | 73.828 | 9.882 | 78.968 | 1.00 | 38.53 | B |
| ATOM | 3250 | CD1 | LEU B | 552 | 73.257 | 10.693 | 80.130 | 1.00 | 38.23 | B |
| ATOM | 3251 | CD2 | LEU B | 552 | 73.327 | 10.416 | 77.631 | 1.00 | 37.33 | B |
| ATOM | 3252 | C | LEU B | 552 | 77.591 | 8.888 | 79.253 | 1.00 | 40.99 | B |
| ATOM | 3253 | O | LEU B | 552 | 78.225 | 9.325 | 80.213 | 1.00 | 41.44 | B |
| ATOM | 3254 | N | ILE B | 553 | 78.160 | 8.579 | 78.093 | 1.00 | 41.46 | B |
| ATOM | 3255 | CA | ILE B | 553 | 79.589 | 8.788 | 77.879 | 1.00 | 41.69 | B |
| ATOM | 3256 | CB | ILE B | 553 | 80.366 | 7.452 | 77.679 | 1.00 | 41.75 | B |
| ATOM | 3257 | CG2 | ILE B | 553 | 81.854 | 7.746 | 77.598 | 1.00 | 41.32 | B |
| ATOM | 3258 | CG1 | ILE B | 553 | 80.077 | 6.451 | 78.811 | 1.00 | 42.93 | B |
| ATOM | 3259 | CD1 | ILE B | 553 | 80.616 | 6.835 | 80.176 | 1.00 | 42.43 | B |
| ATOM | 3260 | C | ILE B | 553 | 79.774 | 9.615 | 76.612 | 1.00 | 41.85 | B |
| ATOM | 3261 | O | ILE B | 553 | 79.057 | 9.419 | 75.631 | 1.00 | 41.17 | B |
| ATOM | 3262 | N | VAL B | 554 | 80.724 | 10.547 | 76.638 | 1.00 | 42.67 | B |
| ATOM | 3263 | CA | VAL B | 554 | 81.022 | 11.371 | 75.467 | 1.00 | 43.83 | B |
| ATOM | 3264 | CB | VAL B | 554 | 81.216 | 12.850 | 75.832 | 1.00 | 43.23 | B |
| ATOM | 3265 | CG1 | VAL B | 554 | 81.219 | 13.690 | 74.566 | 1.00 | 41.99 | B |
| ATOM | 3266 | CG2 | VAL B | 554 | 80.143 | 13.294 | 76.799 | 1.00 | 43.78 | B |
| ATOM | 3267 | C | VAL B | 554 | 82.344 | 10.858 | 74.907 | 1.00 | 44.98 | B |
| ATOM | 3268 | O | VAL B | 554 | 83.391 | 11.108 | 75.489 | 1.00 | 45.12 | B |
| ATOM | 3269 | N | TRP B | 555 | 82.305 | 10.152 | 73.782 | 1.00 | 46.53 | B |
| ATOM | 3270 | CA | TRP B | 555 | 83.526 | 9.603 | 73.198 | 1.00 | 48.33 | B |
| ATOM | 3271 | CB | TRP B | 555 | 83.264 | 8.193 | 72.652 | 1.00 | 48.73 | B |
| ATOM | 3272 | CG | TRP B | 555 | 82.491 | 7.287 | 73.562 | 1.00 | 48.42 | B |
| ATOM | 3273 | CD2 | TRP B | 555 | 83.024 | 6.244 | 74.390 | 1.00 | 49.05 | B |
| ATOM | 3274 | CE2 | TRP B | 555 | 81.934 | 5.644 | 75.060 | 1.00 | 49.51 | B |
| ATOM | 3275 | CE3 | TRP B | 555 | 84.317 | 5.758 | 74.629 | 1.00 | 48.35 | B |
| ATOM | 3276 | CD1 | TRP B | 555 | 81.140 | 7.281 | 73.762 | 1.00 | 48.53 | B |
| ATOM | 3277 | NE1 | TRP B | 555 | 80.797 | 6.296 | 74.661 | 1.00 | 49.31 | B |
| ATOM | 3278 | CZ2 | TRP B | 555 | 82.099 | 4.579 | 75.958 | 1.00 | 49.91 | B |
| ATOM | 3279 | CZ3 | TRP B | 555 | 84.481 | 4.698 | 75.523 | 1.00 | 47.73 | B |
| ATOM | 3280 | CR2 | TRP B | 555 | 83.378 | 4.121 | 76.174 | 1.00 | 48.67 | B |
| ATOM | 3281 | C | TRP B | 555 | 84.119 | 10.455 | 72.074 | 1.00 | 49.09 | B |
| ATOM | 3282 | O | TRP B | 555 | 83.424 | 11.253 | 71.451 | 1.00 | 48.46 | B |
| ATOM | 3283 | N | ASP B | 556 | 85.411 | 10.265 | 71.816 | 1.00 | 51.15 | B |
| ATOM | 3284 | CA | ASP B | 556 | 86.119 | 10.978 | 70.745 | 1.00 | 52.97 | B |
| ATOM | 3285 | CB | ASP B | 556 | 87.358 | 11.705 | 71.295 | 1.00 | 53.47 | B |
| ATOM | 3286 | CG | ASP B | 556 | 88.047 | 12.594 | 70.247 | 1.00 | 53.63 | B |
| ATOM | 3287 | OD1 | ASP B | 556 | 88.097 | 12.203 | 69.060 | 1.00 | 52.44 | B |
| ATOM | 3288 | OD2 | ASP B | 556 | 88.552 | 13.683 | 70.615 | 1.00 | 53.83 | B |
| ATOM | 3289 | C | ASP B | 556 | 86.569 | 9.933 | 69.726 | 1.00 | 53.38 | B |
| ATOM | 3290 | O | ASP B | 556 | 87.740 | 9.560 | 69.688 | 1.00 | 54.18 | B |
| ATOM | 3291 | N | VAL B | 557 | 85.635 | 9.460 | 68.909 | 1.00 | 54.03 | B |
| ATOM | 3292 | CA | VAL B | 557 | 85.925 | 8.446 | 67.896 | 1.00 | 53.93 | B |
| ATOM | 3293 | CB | VAL B | 557 | 84.859 | 8.493 | 66.780 | 1.00 | 53.70 | B |
| ATOM | 3294 | CG1 | VAL B | 557 | 85.210 | 7.519 | 65.660 | 1.00 | 54.83 | B |
| ATOM | 3295 | CG2 | VAL B | 557 | 83.502 | 8.151 | 67.370 | 1.00 | 52.75 | B |
| ATOM | 3296 | C | VAL B | 557 | 87.327 | 8.578 | 67.295 | 1.00 | 54.01 | B |
| ATOM | 3297 | O | VAL B | 557 | 88.014 | 7.575 | 67.065 | 1.00 | 53.12 | B |
| ATOM | 3298 | N | ALA B | 558 | 87.752 | 9.817 | 67.063 | 1.00 | 54.57 | B |
| ATOM | 3299 | CA | ALA B | 558 | 89.071 | 10.087 | 66.497 | 1.00 | 55.27 | B |
| ATOM | 3300 | CB | ALA B | 558 | 89.249 | 11.577 | 66.286 | 1.00 | 54.44 | B |
| ATOM | 3301 | C | ALA B | 558 | 90.189 | 9.554 | 67.393 | 1.00 | 56.09 | B |
| ATOM | 3302 | O | ALA B | 558 | 91.154 | 8.948 | 66.917 | 1.00 | 57.14 | B |
| ATOM | 3303 | N | GLN B | 559 | 90.054 | 9.779 | 68.693 | 1.00 | 56.27 | B |
| ATOM | 3304 | CA | GLN B | 559 | 91.052 | 9.325 | 69.652 | 1.00 | 56.00 | B |
| ATOM | 3305 | CB | GLN B | 559 | 91.358 | 10.445 | 70.643 | 1.00 | 57.58 | B |
| ATOM | 3306 | CG | GLN B | 559 | 91.679 | 11.771 | 69.977 | 1.00 | 60.76 | B |
| ATOM | 3307 | CD | GLN B | 559 | 92.288 | 12.773 | 70.944 | 1.00 | 63.21 | B |
| ATOM | 3308 | OE1 | GLN B | 559 | 91.798 | 12.942 | 72.072 | 1.00 | 63.29 | B |
| ATOM | 3309 | NE2 | GLN B | 559 | 93.360 | 13.449 | 70.510 | 1.00 | 62.95 | B |
| ATOM | 3310 | C | GLN B | 559 | 90.556 | 8.090 | 70.400 | 1.00 | 54.82 | B |
| ATOM | 3311 | O | CLN B | 559 | 91.131 | 7.679 | 71.407 | 1.00 | 53.08 | B |
| ATOM | 3312 | N | MSE B | 560 | 89.486 | 7.500 | 69.885 | 1.00 | 54.44 | B |
| ATOM | 3313 | CA | MSE B | 560 | 88.881 | 6.322 | 70.487 | 1.00 | 54.74 | B |
| ATOM | 3314 | CB | MSE B | 560 | 89.450 | 5.055 | 69.844 | 1.00 | 54.65 | B |
| ATOM | 3315 | CG | MSE B | 560 | 89.124 | 4.912 | 68.360 | 1.00 | 54.73 | B |
| ATOM | 3316 | SE | MSE B | 560 | 87.225 | 4.845 | 67.991 | 1.00 | 57.83 | B |
| ATOM | 3317 | CE | MSE B | 560 | 86.882 | 2.985 | 68.379 | 1.00 | 53.96 | B |
| ATOM | 3318 | C | MSE B | 560 | 89.041 | 6.275 | 72.013 | 1.00 | 54.33 | B |
| ATOM | 3319 | O | MSE B | 560 | 89.330 | 5.230 | 72.600 | 1.00 | 54.09 | B |
| ATOM | 3320 | N | LYS B | 561 | 88.838 | 7.419 | 72.654 | 1.00 | 53.76 | B |
| ATOM | 3321 | CA | LYS B | 561 | 88.945 | 7.494 | 74.100 | 1.00 | 53.03 | B |
| ATOM | 3322 | CB | LYS B | 561 | 90.168 | 8.331 | 74.473 | 0.00 | 53.61 | B |
| ATOM | 3323 | CG | LYS B | 561 | 90.538 | 8.294 | 75.933 | 0.00 | 54.07 | B |
| ATOM | 3324 | CD | LYS B | 561 | 91.867 | 8.981 | 76.146 | 0.00 | 54.52 | B |
| ATOM | 3325 | CE | LYS B | 561 | 92.219 | 9.039 | 77.612 | 0.00 | 54.82 | B |
| ATOM | 3326 | NZ | LYS B | 561 | 91.225 | 9.852 | 78.361 | 0.00 | 55.11 | B |
| ATOM | 3327 | C | LYS B | 561 | 87.663 | 8.092 | 74.690 | 1.00 | 52.48 | B |
| ATOM | 3328 | O | LYS B | 561 | 86.786 | 8.548 | 73.952 | 1.00 | 53.43 | B |
| ATOM | 3329 | N | CYS B | 562 | 87.553 | 8.063 | 76.018 | 1.00 | 50.98 | B |
| ATOM | 3330 | CA | CYS B | 562 | 86.394 | 8.601 | 76.740 | 1.00 | 48.94 | B |
| ATOM | 3331 | CB | CYS B | 562 | 86.209 | 7.834 | 78.056 | 1.00 | 49.61 | B |
| ATOM | 3332 | SG | CYS B | 562 | 85.037 | 8.536 | 79.259 | 1.00 | 51.47 | B |
| ATOM | 3333 | C | CYS B | 562 | 86.646 | 10.076 | 77.029 | 1.00 | 47.64 | B |
| ATOM | 3334 | O | CYS B | 562 | 87.772 | 10.450 | 77.339 | 1.00 | 48.22 | B |
| ATOM | 3335 | N | LEU B | 563 | 85.616 | 10.915 | 76.909 | 1.00 | 46.12 | B |
| ATOM | 3336 | CA | LEU B | 563 | 85.771 | 12.346 | 77.175 | 1.00 | 44.98 | B |
| ATOM | 3337 | CB | LEU B | 563 | 85.149 | 13.187 | 76.051 | 1.00 | 44.96 | B |
| ATOM | 3338 | CG | LEU B | 563 | 85.894 | 13.179 | 74.706 | 1.00 | 46.73 | B |
| ATOM | 3339 | CD1 | LEU B | 563 | 85.234 | 14.142 | 73.721 | 1.00 | 45.90 | B |
| ATOM | 3340 | CD2 | LEU B | 563 | 87.346 | 13.583 | 74.931 | 1.00 | 45.81 | B |
| ATOM | 3341 | C | LEU B | 563 | 85.165 | 12.731 | 78.518 | 1.00 | 43.88 | B |
| ATOM | 3342 | O | LEU B | 563 | 85.823 | 13.367 | 79.348 | 1.00 | 43.62 | B |
| ATOM | 3343 | N | TYR B | 564 | 83.913 | 12.345 | 78.731 | 1.00 | 42.63 | B |
| ATOM | 3344 | CA | TYR B | 564 | 83.244 | 12.632 | 79.986 | 1.00 | 41.64 | B |
| ATOM | 3345 | CB | TYR B | 564 | 82.429 | 13.918 | 79.887 | 1.00 | 41.63 | B |
| ATOM | 3346 | CG | TYR B | 564 | 83.248 | 15.149 | 79.592 | 1.00 | 42.86 | B |
| ATOM | 3347 | CD1 | TYR B | 564 | 83.298 | 15.684 | 78.308 | 1.00 | 43.33 | B |
| ATOM | 3348 | CE1 | TYR B | 564 | 84.049 | 16.819 | 78.036 | 1.00 | 45.08 | B |
| ATOM | 3349 | CD2 | TYR B | 564 | 83.976 | 15.782 | 80.602 | 1.00 | 43.76 | B |
| ATOM | 3350 | CE2 | TYR B | 564 | 84.732 | 16.916 | 80.341 | 1.00 | 43.78 | B |
| ATOM | 3351 | CZ | TYR B | 564 | 84.760 | 17.426 | 79.060 | 1.00 | 46.05 | B |
| ATOM | 3352 | OH | TYR B | 564 | 85.492 | 18.558 | 78.795 | 1.00 | 48.58 | B |
| ATOM | 3353 | C | TYR B | 564 | 82.321 | 11.491 | 80.356 | 1.00 | 41.77 | B |
| ATOM | 3354 | O | TYR B | 564 | 81.899 | 10.722 | 79.489 | 1.00 | 42.27 | B |
| ATOM | 3355 | N | ILE B | 565 | 82.011 | 11.391 | 81.647 | 1.00 | 41.33 | B |
| ATOM | 3356 | CA | ILE B | 565 | 81.116 | 10.358 | 82.156 | 1.00 | 40.82 | B |
| ATOM | 3357 | CB | ILE B | 565 | 81.828 | 9.414 | 83.169 | 1.00 | 41.48 | B |
| ATOM | 3358 | CG2 | ILE B | 565 | 80.934 | 8.234 | 83.486 | 1.00 | 40.38 | B |
| ATOM | 3359 | CG1 | ILE B | 565 | 83.126 | 8.862 | 82.582 | 1.00 | 40.59 | B |
| ATOM | 3360 | CD1 | ILE B | 565 | 83.907 | 8.008 | 83.571 | 1.00 | 41.08 | B |
| ATOM | 3361 | C | ILE B | 565 | 79.938 | 11.020 | 82.860 | 1.00 | 40.09 | B |
| ATOM | 3362 | O | ILE B | 565 | 79.853 | 11.020 | 84.089 | 1.00 | 41.84 | B |
| ATOM | 3363 | N | LEU B | 566 | 79.033 | 11.582 | 82.067 | 1.00 | 38.80 | B |
| ATOM | 3364 | CA | LEU B | 566 | 77.841 | 12.256 | 82.580 | 1.00 | 36.98 | B |
| ATOM | 3365 | CB | LEU B | 566 | 76.990 | 12.754 | 81.421 | 1.00 | 36.01 | B |
| ATOM | 3366 | CG | LEU B | 566 | 77.792 | 13.369 | 80.272 | 1.00 | 35.51 | B |
| ATOM | 3367 | CD1 | LEU B | 566 | 76.846 | 13.709 | 79.138 | 1.00 | 34.67 | B |
| ATOM | 3368 | CD2 | LEU B | 566 | 78.541 | 14.592 | 80.757 | 1.00 | 33.68 | B |
| ATOM | 3369 | C | LEU B | 566 | 77.010 | 11.306 | 83.411 | 1.00 | 36.96 | B |
| ATOM | 3370 | O | LEU B | 566 | 76.354 | 10.414 | 82.877 | 1.00 | 36.93 | B |
| ATOM | 3371 | N | SER B | 567 | 77.021 | 11.498 | 84.721 | 1.00 | 37.95 | B |
| ATOM | 3372 | CA | SER B | 567 | 76.252 | 10.628 | 85.601 | 1.00 | 39.30 | B |
| ATOM | 3373 | CB | SER B | 567 | 77.191 | 9.655 | 86.305 | 1.00 | 39.73 | B |
| ATOM | 3374 | OG | SER B | 567 | 78.238 | 10.359 | 86.940 | 1.00 | 40.54 | B |
| ATOM | 3375 | C | SER B | 567 | 75.466 | 11.417 | 86.635 | 1.00 | 39.56 | B |
| ATOM | 3376 | O | SER B | 567 | 76.007 | 12.260 | 87.344 | 1.00 | 39.82 | B |
| ATOM | 3377 | N | GLY B | 568 | 74.181 | 11.116 | 86.728 | 1.00 | 40.79 | B |
| ATOM | 3378 | CA | GLY B | 568 | 73.338 | 11.798 | 87.685 | 1.00 | 40.25 | B |
| ATOM | 3379 | C | GLY B | 568 | 71.949 | 11.190 | 87.704 | 1.00 | 40.41 | B |
| ATOM | 3380 | O | GLY B | 568 | 71.270 | 11.232 | 88.723 | 1.00 | 41.14 | B |
| ATOM | 3381 | N | HIS B | 569 | 71.513 | 10.636 | 86.575 | 1.00 | 40.65 | B |
| ATOM | 3382 | CA | HIS B | 569 | 70.197 | 10.017 | 86.508 | 1.00 | 40.96 | B |
| ATOM | 3383 | CB | HIS B | 569 | 69.865 | 9.568 | 85.077 | 1.00 | 40.33 | B |
| ATOM | 3384 | CG | HIS B | 569 | 69.511 | 10.695 | 84.151 | 1.00 | 40.43 | B |
| ATOM | 3385 | CD2 | HIS B | 569 | 68.558 | 11.657 | 84.232 | 1.00 | 39.98 | B |
| ATOM | 3386 | ND1 | HIS B | 569 | 70.194 | 10.936 | 82.977 | 1.00 | 40.06 | B |
| ATOM | 3387 | CE1 | HIS B | 569 | 69.681 | 11.995 | 82.378 | 1.00 | 38.53 | B |
| ATOM | 3388 | NE2 | HIS B | 569 | 68.687 | 12.452 | 83.118 | 1.00 | 38.90 | B |
| ATOM | 3389 | C | HIS B | 569 | 70.193 | 8.822 | 87.448 | 1.00 | 40.82 | B |
| ATOM | 3390 | O | HIS B | 569 | 71.239 | 8.242 | 87.743 | 1.00 | 39.68 | B |
| ATOM | 3391 | N | THR B | 570 | 69.007 | 8.464 | 87.915 | 1.00 | 41.28 | B |
| ATOM | 3392 | CA | THR B | 570 | 68.857 | 7.364 | 88.838 | 1.00 | 41.75 | B |
| ATOM | 3393 | CB | THR B | 570 | 68.025 | 7.806 | 90.042 | 1.00 | 42.58 | B |
| ATOM | 3394 | OG1 | THR B | 570 | 68.417 | 9.135 | 90.414 | 1.00 | 41.17 | B |
| ATOM | 3395 | CG2 | THR B | 570 | 68.265 | 6.872 | 91.226 | 1.00 | 44.09 | B |
| ATOM | 3396 | C | THR B | 570 | 68.201 | 6.169 | 88.168 | 1.00 | 41.63 | B |
| ATOM | 3397 | O | THR B | 570 | 67.554 | 5.348 | 88.824 | 1.00 | 42.87 | B |
| ATOM | 3398 | N | ASP B | 571 | 68.348 | 6.087 | 86.852 | 1.00 | 40.41 | B |
| ATOM | 3399 | CA | ASP B | 571 | 67.795 | 4.969 | 86.116 | 1.00 | 38.73 | B |
| ATOM | 3400 | CB | ASP B | 571 | 66.273 | 4.960 | 86.200 | 1.00 | 37.92 | B |
| ATOM | 3401 | CG | ASP B | 571 | 65.726 | 3.569 | 86.446 | 1.00 | 38.68 | B |
| ATOM | 3402 | OD1 | ASP B | 571 | 66.270 | 2.616 | 85.852 | 1.00 | 37.31 | B |
| ATOM | 3403 | OD2 | ASP B | 571 | 64.757 | 3.425 | 87.225 | 1.00 | 39.84 | B |
| ATOM | 3404 | C | ASP B | 571 | 68.240 | 4.941 | 84.663 | 1.00 | 38.21 | B |
| ATOM | 3405 | O | ASP B | 571 | 68.800 | 5.914 | 84.147 | 1.00 | 37.05 | B |
| ATOM | 3406 | N | ARG B | 572 | 67.994 | 3.806 | 84.016 | 1.00 | 38.06 | B |
| ATOM | 3407 | CA | ARG B | 572 | 68.365 | 3.602 | 82.620 | 1.00 | 38.61 | B |
| ATOM | 3408 | CB | ARG B | 572 | 67.761 | 2.282 | 82.102 | 1.00 | 39.79 | B |
| ATOM | 3409 | CG | ARG B | 572 | 66.409 | 1.944 | 82.735 | 1.00 | 42.08 | B |
| ATOM | 3410 | CD | ARG B | 572 | 65.669 | 0.771 | 82.066 | 1.00 | 43.20 | B |
| ATOM | 3411 | NE | ARG B | 572 | 66.402 | −0.497 | 82.046 | 1.00 | 42.53 | B |
| ATOM | 3412 | CZ | ARG B | 572 | 65.859 | −1.671 | 81.722 | 1.00 | 42.93 | B |
| ATOM | 3413 | NH1 | ARG B | 572 | 64.573 | −1.757 | 81.395 | 1.00 | 41.39 | B |
| ATOM | 3414 | NH2 | ARG B | 572 | 66.605 | −2.764 | 81.708 | 1.00 | 43.19 | B |
| ATOM | 3415 | C | ARG B | 572 | 67.961 | 4.764 | 81.712 | 1.00 | 37.29 | B |
| ATOM | 3416 | O | ARG B | 572 | 66.923 | 5.408 | 81.911 | 1.00 | 37.19 | B |
| ATOM | 3417 | N | ILE B | 573 | 68.803 | 5.022 | 80.716 | 1.00 | 34.87 | B |
| ATOM | 3418 | CA | ILE B | 573 | 68.589 | 6.088 | 79.739 | 1.00 | 32.30 | B |
| ATOM | 3419 | CB | ILE B | 573 | 69.914 | 6.783 | 79.417 | 1.00 | 31.72 | B |
| ATOM | 3420 | CG2 | ILE B | 573 | 69.736 | 7.751 | 78.276 | 1.00 | 29.59 | B |
| ATOM | 3421 | CG1 | ILE B | 573 | 70.434 | 7.465 | 80.678 | 1.00 | 33.36 | B |
| ATOM | 3422 | CD1 | ILE B | 573 | 71.875 | 7.918 | 80.574 | 1.00 | 34.32 | B |
| ATOM | 3423 | C | ILE B | 573 | 68.024 | 5.489 | 78.454 | 1.00 | 30.51 | B |
| ATOM | 3424 | O | ILE B | 573 | 68.467 | 4.441 | 78.009 | 1.00 | 29.57 | B |
| ATOM | 3425 | N | TYR B | 574 | 67.039 | 6.148 | 77.862 | 1.00 | 29.36 | B |
| ATOM | 3426 | CA | TYR B | 574 | 66.453 | 5.629 | 76.638 | 1.00 | 29.00 | B |
| ATOM | 3427 | CB | TYR B | 574 | 64.944 | 5.478 | 76.795 | 1.00 | 29.33 | B |
| ATOM | 3428 | CG | TYR B | 574 | 64.557 | 4.229 | 77.537 | 1.00 | 31.08 | B |
| ATOM | 3429 | CD1 | TYR B | 574 | 65.140 | 3.926 | 78.754 | 1.00 | 33.15 | B |
| ATOM | 3430 | CE1 | TYR B | 574 | 64.782 | 2.797 | 79.465 | 1.00 | 33.55 | B |
| ATOM | 3431 | CD2 | TYR B | 574 | 63.596 | 3.358 | 77.036 | 1.00 | 32.28 | B |
| ATOM | 3432 | CE2 | TYR B | 574 | 63.225 | 2.209 | 77.748 | 1.00 | 32.21 | B |
| ATOM | 3433 | CZ | TYR B | 574 | 63.828 | 1.947 | 78.970 | 1.00 | 32.73 | B |
| ATOM | 3434 | OH | TYR B | 574 | 63.457 | 0.871 | 79.743 | 1.00 | 34.36 | B |
| ATOM | 3435 | C | TYR B | 574 | 66.745 | 6.452 | 75.393 | 1.00 | 28.82 | B |
| ATOM | 3436 | O | TYR B | 574 | 66.611 | 5.965 | 74.264 | 1.00 | 28.32 | B |
| ATOM | 3437 | N | SER B | 575 | 67.169 | 7.692 | 75.590 | 1.00 | 28.07 | B |
| ATOM | 3438 | CA | SER B | 575 | 67.431 | 8.564 | 74.461 | 1.00 | 27.39 | B |
| ATOM | 3439 | CB | SER B | 575 | 66.101 | 9.147 | 73.980 | 1.00 | 27.09 | B |
| ATOM | 3440 | OC | SER B | 575 | 66.260 | 9.948 | 72.833 | 1.00 | 23.40 | B |
| ATOM | 3441 | C | SER B | 575 | 68.368 | 9.690 | 74.832 | 1.00 | 27.21 | B |
| ATOM | 3442 | O | SER B | 575 | 68.400 | 10.124 | 75.979 | 1.00 | 29.42 | B |
| ATOM | 3443 | N | THR B | 576 | 69.131 | 10.169 | 73.859 | 1.00 | 26.42 | B |
| ATOM | 3444 | CA | THR B | 576 | 70.036 | 11.279 | 74.101 | 1.00 | 24.44 | B |
| ATOM | 3445 | CB | THR B | 576 | 71.350 | 10.794 | 74.680 | 1.00 | 23.42 | B |
| ATOM | 3446 | OG1 | THR B | 576 | 72.176 | 11.925 | 74.958 | 1.00 | 22.28 | B |
| ATOM | 3447 | CG2 | THR B | 576 | 72.070 | 9.898 | 73.680 | 1.00 | 23.95 | B |
| ATOM | 3448 | C | THR B | 576 | 70.314 | 11.995 | 72.782 | 1.00 | 24.66 | B |
| ATOM | 3449 | O | THR B | 576 | 70.225 | 11.405 | 71.712 | 1.00 | 23.66 | B |
| ATOM | 3450 | N | ILE B | 577 | 70.639 | 13.274 | 72.843 | 1.00 | 25.19 | B |
| ATOM | 3451 | CA | ILE B | 577 | 70.938 | 13.979 | 71.615 | 1.00 | 24.85 | B |
| ATOM | 3452 | CB | ILE B | 577 | 69.767 | 14.869 | 71.172 | 1.00 | 23.89 | B |
| ATOM | 3453 | CG2 | ILE B | 577 | 70.271 | 15.939 | 70.202 | 1.00 | 21.80 | B |
| ATOM | 3454 | CG1 | ILE B | 577 | 68.671 | 14.011 | 70.538 | 1.00 | 23.81 | B |
| ATOM | 3455 | CD1 | ILE B | 577 | 67.376 | 14.750 | 70.224 | 1.00 | 21.31 | B |
| ATOM | 3456 | C | ILE B | 577 | 72.163 | 14.851 | 71.802 | 1.00 | 26.44 | B |
| ATOM | 3457 | O | ILE B | 577 | 72.324 | 15.497 | 72.833 | 1.00 | 26.92 | B |
| ATOM | 3458 | N | TYR B | 578 | 73.037 | 14.850 | 70.802 | 1.00 | 27.45 | B |
| ATOM | 3459 | CA | TYR B | 578 | 74.235 | 15.672 | 70.848 | 1.00 | 27.93 | B |
| ATOM | 3460 | CB | TYR B | 578 | 75.423 | 14.925 | 70.226 | 1.00 | 28.17 | B |
| ATOM | 3461 | CG | TYR B | 578 | 76.688 | 15.757 | 70.084 | 1.00 | 30.25 | B |
| ATOM | 3462 | CD1 | TYR B | 578 | 77.096 | 16.629 | 71.089 | 1.00 | 28.62 | B |
| ATOM | 3463 | CE1 | TYR B | 578 | 78.235 | 17.416 | 70.934 | 1.00 | 29.38 | B |
| ATOM | 3464 | CD2 | TYR B | 578 | 77.464 | 15.690 | 68.922 | 1.00 | 31.91 | B |
| ATOM | 3465 | CE2 | TYR B | 578 | 78.607 | 16.476 | 68.762 | 1.00 | 30.14 | B |
| ATOM | 3466 | CZ | TYR B | 578 | 78.980 | 17.334 | 69.767 | 1.00 | 29.57 | B |
| ATOM | 3467 | OH | TYR B | 578 | 80.082 | 18.135 | 69.605 | 1.00 | 31.32 | B |
| ATOM | 3468 | C | TYR B | 578 | 73.943 | 16.980 | 70.117 | 1.00 | 27.87 | B |
| ATOM | 3469 | O | TYR B | 578 | 73.789 | 17.017 | 68.897 | 1.00 | 27.54 | B |
| ATOM | 3470 | N | ASP B | 579 | 73.832 | 18.045 | 70.901 | 1.00 | 28.90 | B |
| ATOM | 3471 | CA | ASP B | 579 | 73.551 | 19.369 | 70.380 | 1.00 | 30.92 | B |
| ATOM | 3472 | CB | ASP B | 579 | 72.799 | 20.203 | 71.416 | 1.00 | 30.53 | B |
| ATOM | 3473 | CG | ASP B | 579 | 72.248 | 21.473 | 70.839 | 1.00 | 34.02 | B |
| ATOM | 3474 | OD1 | ASP B | 579 | 72.845 | 21.995 | 69.868 | 1.00 | 35.60 | B |
| ATOM | 3475 | OD2 | ASP B | 579 | 71.219 | 21.968 | 71.357 | 1.00 | 37.17 | B |
| ATOM | 3476 | C | ASP B | 579 | 74.871 | 20.026 | 70.035 | 1.00 | 31.94 | B |
| ATOM | 3477 | O | ASP B | 579 | 75.344 | 20.923 | 70.732 | 1.00 | 30.49 | B |
| ATOM | 3478 | N | HIS B | 580 | 75.461 | 19.557 | 68.945 | 1.00 | 34.88 | B |
| ATOM | 3479 | CA | HIS B | 580 | 76.731 | 20.085 | 68.492 | 1.00 | 38.67 | B |
| ATOM | 3480 | CB | HIS B | 580 | 77.133 | 19.422 | 67.170 | 1.00 | 40.80 | B |
| ATOM | 3481 | CG | HIS B | 580 | 76.123 | 19.579 | 66.080 | 1.00 | 44.94 | B |
| ATOM | 3482 | CD2 | HIS B | 580 | 76.268 | 19.900 | 64.773 | 1.00 | 46.55 | B |
| ATOM | 3483 | ND1 | HIS B | 580 | 74.778 | 19.359 | 66.276 | 1.00 | 47.64 | B |
| ATOM | 3484 | CE1 | HIS B | 580 | 74.135 | 19.532 | 65.132 | 1.00 | 47.95 | B |
| ATOM | 3485 | NE2 | HIS B | 580 | 75.016 | 19.860 | 64.206 | 1.00 | 46.75 | B |
| ATOM | 3486 | C | HIS B | 580 | 76.684 | 21.601 | 68.364 | 1.00 | 39.17 | B |
| ATOM | 3487 | O | HIS B | 580 | 77.545 | 22.297 | 68.907 | 1.00 | 40.61 | B |
| ATOM | 3488 | N | GLU B | 581 | 75.673 | 22.122 | 67.681 | 1.00 | 39.97 | B |
| ATOM | 3489 | CA | GLU B | 581 | 75.563 | 23.566 | 67.529 | 1.00 | 40.63 | B |
| ATOM | 3490 | CB | GLU B | 581 | 74.162 | 23.960 | 67.078 | 1.00 | 42.60 | B |
| ATOM | 3491 | CG | GLU B | 581 | 73.991 | 25.469 | 66.960 | 1.00 | 46.26 | B |
| ATOM | 3492 | CD | GLU B | 581 | 72.848 | 25.853 | 66.052 | 1.00 | 47.68 | B |
| ATOM | 3493 | OE1 | GLU B | 581 | 72.857 | 25.407 | 64.880 | 1.00 | 48.93 | B |
| ATOM | 3494 | OE2 | GLU B | 581 | 71.952 | 26.598 | 66.508 | 1.00 | 48.37 | B |
| ATOM | 3495 | C | GLU B | 581 | 75.871 | 24.279 | 68.836 | 1.00 | 40.05 | B |
| ATOM | 3496 | O | GLU B | 581 | 76.810 | 25.063 | 68.916 | 1.00 | 40.37 | B |
| ATOM | 3497 | N | ARG B | 582 | 75.074 | 23.998 | 69.859 | 1.00 | 39.74 | B |
| ATOM | 3498 | CA | ARG B | 582 | 75.266 | 24.622 | 71.151 | 1.00 | 38.74 | B |
| ATOM | 3499 | CB | ARG B | 582 | 73.939 | 24.647 | 71.899 | 1.00 | 37.10 | B |
| ATOM | 3500 | CG | ARG B | 582 | 73.131 | 25.905 | 71.619 | 1.00 | 37.06 | B |
| ATOM | 3501 | CD | ARG B | 582 | 71.654 | 25.749 | 71.970 | 1.00 | 35.83 | B |
| ATOM | 3502 | NE | ARG B | 582 | 70.962 | 24.890 | 71.012 | 1.00 | 35.08 | B |
| ATOM | 3503 | CZ | ARG B | 582 | 70.041 | 25.312 | 70.154 | 1.00 | 34.96 | B |
| ATOM | 3504 | NH1 | ARG B | 582 | 69.691 | 26.591 | 70.130 | 1.00 | 32.75 | B |
| ATOM | 3505 | NH2 | ARG B | 582 | 69.466 | 24.448 | 69.327 | 1.00 | 35.91 | B |
| ATOM | 3506 | C | ARG B | 582 | 76.356 | 23.971 | 71.989 | 1.00 | 39.71 | B |
| ATOM | 3507 | O | ARG B | 582 | 76.775 | 24.529 | 73.003 | 1.00 | 40.75 | B |
| ATOM | 3508 | N | LYS B | 583 | 76.825 | 22.804 | 71.548 | 1.00 | 40.22 | B |
| ATOM | 3509 | CA | LYS B | 583 | 77.881 | 22.056 | 72.240 | 1.00 | 39.65 | B |
| ATOM | 3510 | CB | LYS B | 583 | 79.089 | 22.961 | 72.487 | 1.00 | 41.30 | B |
| ATOM | 3511 | CG | LYS B | 583 | 80.401 | 22.212 | 72.649 | 1.00 | 43.06 | B |
| ATOM | 3512 | CD | LYS B | 583 | 80.866 | 21.664 | 71.311 | 1.00 | 46.02 | B |
| ATOM | 3513 | CE | LYS B | 583 | 81.196 | 22.806 | 70.367 | 1.00 | 46.16 | B |
| ATOM | 3514 | NZ | LYS B | 583 | 82.256 | 23.657 | 70.976 | 1.00 | 46.38 | B |
| ATOM | 3515 | C | LYS B | 583 | 77.357 | 21.524 | 73.574 | 1.00 | 38.48 | B |
| ATOM | 3516 | O | LYS B | 583 | 77.990 | 21.689 | 74.615 | 1.00 | 37.90 | B |
| ATOM | 3517 | N | ARG B | 584 | 76.202 | 20.871 | 73.526 | 1.00 | 36.80 | B |
| ATOM | 3518 | CA | ARG B | 584 | 75.575 | 20.343 | 74.728 | 1.00 | 36.72 | B |
| ATOM | 3519 | CB | ARG B | 584 | 74.436 | 21.278 | 75.140 | 1.00 | 35.58 | B |
| ATOM | 3520 | CG | ARG B | 584 | 74.930 | 22.481 | 75.916 | 1.00 | 38.40 | B |
| ATOM | 3521 | CD | ARG B | 584 | 74.542 | 23.820 | 75.330 | 1.00 | 37.40 | B |
| ATOM | 3522 | NE | ARG B | 584 | 73.160 | 24.166 | 75.635 | 1.00 | 39.31 | B |
| ATOM | 3523 | CZ | ARG B | 584 | 72.723 | 25.408 | 75.833 | 1.00 | 40.87 | B |
| ATOM | 3524 | NH1 | ARG B | 584 | 73.556 | 26.437 | 75.759 | 1.00 | 40.61 | B |
| ATOM | 3525 | NH2 | ARG B | 584 | 71.448 | 25.617 | 76.122 | 1.00 | 40.77 | B |
| ATOM | 3526 | C | ARG B | 584 | 75.045 | 18.915 | 74.573 | 1.00 | 36.65 | B |
| ATOM | 3527 | O | ARG B | 584 | 75.211 | 18.298 | 73.522 | 1.00 | 36.06 | B |
| ATOM | 3528 | N | CYS B | 585 | 74.443 | 18.372 | 75.631 | 1.00 | 36.04 | B |
| ATOM | 3529 | CA | CYS B | 585 | 73.847 | 17.043 | 75.534 | 1.00 | 34.78 | B |
| ATOM | 3530 | CB | CYS B | 585 | 74.778 | 15.936 | 75.994 | 1.00 | 36.01 | B |
| ATOM | 3531 | SG | CYS B | 585 | 73.955 | 14.322 | 75.938 | 1.00 | 38.57 | B |
| ATOM | 3532 | C | CYS B | 585 | 72.566 | 16.929 | 76.330 | 1.00 | 33.64 | B |
| ATOM | 3533 | O | CYS B | 585 | 72.503 | 17.328 | 77.495 | 1.00 | 33.83 | B |
| ATOM | 3534 | N | ILE B | 586 | 71.544 | 16.397 | 75.668 | 1.00 | 31.40 | B |
| ATOM | 3535 | CA | ILE B | 586 | 70.247 | 16.182 | 76.277 | 1.00 | 31.62 | B |
| ATOM | 3536 | CB | ILE B | 586 | 69.079 | 16.610 | 75.325 | 1.00 | 29.83 | B |
| ATOM | 3537 | CG2 | ILE B | 586 | 67.725 | 16.305 | 75.961 | 1.00 | 25.17 | B |
| ATOM | 3538 | CG1 | ILE B | 586 | 69.155 | 18.111 | 75.009 | 1.00 | 28.59 | B |
| ATOM | 3539 | CD1 | ILE B | 586 | 69.610 | 18.438 | 73.588 | 1.00 | 27.79 | B |
| ATOM | 3540 | C | ILE B | 586 | 70.135 | 14.680 | 76.534 | 1.00 | 33.85 | B |
| ATOM | 3541 | O | ILE B | 586 | 70.530 | 13.882 | 75.679 | 1.00 | 35.25 | B |
| ATOM | 3542 | N | SER B | 587 | 69.634 | 14.291 | 77.712 | 1.00 | 34.29 | B |
| ATOM | 3543 | CA | SER B | 587 | 69.428 | 12.870 | 78.031 | 1.00 | 34.28 | B |
| ATOM | 3544 | CB | SER B | 587 | 70.548 | 12.320 | 78.933 | 1.00 | 33.74 | B |
| ATOM | 3545 | OG | SER B | 587 | 70.727 | 13.066 | 80.122 | 1.00 | 34.80 | B |
| ATOM | 3546 | C | SER B | 587 | 68.060 | 12.648 | 78.685 | 1.00 | 33.99 | B |
| ATOM | 3547 | O | SER B | 587 | 67.639 | 13.419 | 79.546 | 1.00 | 32.52 | B |
| ATOM | 3548 | N | ALA B | 588 | 67.355 | 11.615 | 78.233 | 1.00 | 34.19 | B |
| ATOM | 3549 | CA | ALA B | 588 | 66.054 | 11.286 | 78.793 | 1.00 | 35.71 | B |
| ATOM | 3550 | CB | ALA B | 588 | 65.006 | 11.155 | 77.699 | 1.00 | 35.73 | B |
| ATOM | 3551 | C | ALA B | 588 | 66.259 | 9.960 | 79.495 | 1.00 | 36.25 | B |
| ATOM | 3552 | O | ALA B | 588 | 66.752 | 9.016 | 78.884 | 1.00 | 37.28 | B |
| ATOM | 3553 | N | SER B | 589 | 65.929 | 9.906 | 80.786 | 1.00 | 36.81 | B |
| ATOM | 3554 | CA | SER B | 589 | 66.084 | 8.684 | 81.582 | 1.00 | 35.90 | B |
| ATOM | 3555 | CB | SER B | 589 | 66.888 | 8.950 | 82.851 | 1.00 | 34.91 | B |
| ATOM | 3556 | OG | SER B | 589 | 66.512 | 8.030 | 83.870 | 1.00 | 32.54 | B |
| ATOM | 3557 | C | SER B | 589 | 64.761 | 8.093 | 82.005 | 1.00 | 35.49 | B |
| ATOM | 3558 | O | SER B | 589 | 63.714 | 8.712 | 81.844 | 1.00 | 34.24 | B |
| ATOM | 3559 | N | MSE B | 590 | 64.826 | 6.883 | 82.550 | 1.00 | 36.42 | B |
| ATOM | 3560 | CA | MSE B | 590 | 63.648 | 6.188 | 83.040 | 1.00 | 36.64 | B |
| ATOM | 3561 | CB | MSE B | 590 | 63.949 | 4.702 | 83.249 | 1.00 | 39.18 | B |
| ATOM | 3562 | CG | MSE B | 590 | 62.896 | 3.948 | 84.060 | 1.00 | 41.10 | B |
| ATOM | 3563 | SE | MSE B | 590 | 63.091 | 2.012 | 83.900 | 1.00 | 46.30 | B |
| ATOM | 3564 | CE | MSE B | 590 | 61.725 | 1.645 | 82.565 | 1.00 | 45.20 | B |
| ATOM | 3565 | C | MSE B | 590 | 63.241 | 6.819 | 84.361 | 1.00 | 35.83 | B |
| ATOM | 3566 | O | MSE B | 590 | 62.152 | 6.554 | 84.860 | 1.00 | 35.48 | B |
| ATOM | 3567 | N | ASP B | 591 | 64.116 | 7.650 | 84.932 | 1.00 | 35.51 | B |
| ATOM | 3568 | CA | ASP B | 591 | 63.791 | 8.301 | 86.198 | 1.00 | 34.59 | B |
| ATOM | 3569 | CB | ASP B | 591 | 65.051 | 8.767 | 86.951 | 1.00 | 34.80 | B |
| ATOM | 3570 | CG | ASP B | 591 | 65.874 | 9.763 | 86.177 | 1.00 | 34.10 | B |
| ATOM | 3571 | OD1 | ASP B | 591 | 65.329 | 10.459 | 85.300 | 1.00 | 35.66 | B |
| ATOM | 3572 | OD2 | ASP B | 591 | 67.081 | 9.869 | 86.466 | 1.00 | 33.64 | B |
| ATOM | 3573 | C | ASP B | 591 | 62.838 | 9.463 | 85.986 | 1.00 | 33.76 | B |
| ATOM | 3574 | O | ASP B | 591 | 62.767 | 10.373 | 86.801 | 1.00 | 31.76 | B |
| ATOM | 3575 | N | THR B | 592 | 62.120 | 9.418 | 84.866 | 1.00 | 35.29 | B |
| ATOM | 3576 | CA | THR B | 592 | 61.120 | 10.419 | 84.536 | 1.00 | 34.85 | B |
| ATOM | 3577 | CB | THR B | 592 | 59.984 | 10.372 | 85.631 | 1.00 | 34.66 | B |
| ATOM | 3578 | OG1 | THR B | 592 | 58.742 | 10.820 | 85.079 | 1.00 | 38.41 | B |
| ATOM | 3579 | CG2 | THR B | 592 | 60.338 | 11.235 | 86.830 | 1.00 | 34.65 | B |
| ATOM | 3580 | C | THR B | 592 | 61.745 | 11.811 | 84.403 | 1.00 | 33.52 | B |
| ATOM | 3581 | O | THR B | 592 | 61.049 | 12.815 | 84.455 | 1.00 | 33.23 | B |
| ATOM | 3582 | N | THR B | 593 | 63.053 | 11.858 | 84.169 | 1.00 | 32.55 | B |
| ATOM | 3583 | CA | THR B | 593 | 63.764 | 13.130 | 84.070 | 1.00 | 32.86 | B |
| ATOM | 3584 | CB | THR B | 593 | 64.786 | 13.239 | 85.217 | 1.00 | 34.19 | B |
| ATOM | 3585 | OG1 | THR B | 593 | 64.082 | 13.389 | 86.454 | 1.00 | 36.50 | B |
| ATOM | 3586 | CG2 | THR B | 593 | 65.730 | 14.430 | 85.018 | 1.00 | 38.69 | B |
| ATOM | 3587 | C | THR B | 593 | 64.500 | 13.423 | 82.765 | 1.00 | 33.21 | B |
| ATOM | 3588 | O | THR B | 593 | 64.856 | 12.519 | 82.015 | 1.00 | 32.79 | B |
| ATOM | 3589 | N | ILE B | 594 | 64.714 | 14.705 | 82.488 | 1.00 | 33.30 | B |
| ATOM | 3590 | CA | ILE B | 594 | 65.471 | 15.103 | 81.309 | 1.00 | 33.34 | B |
| ATOM | 3591 | CB | ILE B | 594 | 64.612 | 15.829 | 80.249 | 1.00 | 31.46 | B |
| ATOM | 3592 | CG2 | ILE B | 594 | 65.496 | 16.322 | 79.132 | 1.00 | 29.37 | B |
| ATOM | 3593 | CO1 | ILE B | 594 | 63.534 | 14.902 | 79.690 | 1.00 | 32.39 | B |
| ATOM | 3594 | CD1 | ILE B | 594 | 62.436 | 15.642 | 78.920 | 1.00 | 27.89 | B |
| ATOM | 3595 | C | ILE B | 594 | 66.497 | 16.104 | 81.824 | 1.00 | 35.21 | B |
| ATOM | 3596 | O | ILE B | 594 | 66.161 | 16.973 | 82.628 | 1.00 | 37.01 | B |
| ATOM | 3597 | N | ARG B | 595 | 67.749 | 15.977 | 81.402 | 1.00 | 35.67 | B |
| ATOM | 3598 | CA | ARG B | 595 | 68.735 | 16.947 | 81.834 | 1.00 | 36.60 | B |
| ATOM | 3599 | CB | ARG B | 595 | 69.447 | 16.482 | 83.104 | 1.00 | 39.57 | B |
| ATOM | 3600 | CO | ARG B | 595 | 70.237 | 15.219 | 83.020 | 1.00 | 41.84 | B |
| ATOM | 3601 | CD | ARG B | 595 | 70.672 | 14.865 | 84.428 | 1.00 | 43.91 | B |
| ATOM | 3602 | NE | ARG B | 595 | 69.554 | 14.397 | 85.240 | 1.00 | 44.22 | B |
| ATOM | 3603 | CZ | ARG B | 595 | 69.658 | 14.054 | 86.518 | 1.00 | 45.26 | B |
| ATOM | 3604 | NH1 | ARG B | 595 | 70.848 | 14.147 | 87.121 | 1.00 | 44.50 | B |
| ATOM | 3605 | NH2 | ARG B | 595 | 68.586 | 13.589 | 87.170 | 1.00 | 41.38 | B |
| ATOM | 3606 | C | ARG B | 595 | 69.731 | 17.324 | 80.751 | 1.00 | 36.11 | B |
| ATOM | 3607 | O | ARG B | 595 | 70.067 | 16.512 | 79.880 | 1.00 | 36.08 | B |
| ATOM | 3608 | N | ILE B | 596 | 70.173 | 18.579 | 80.791 | 1.00 | 34.47 | B |
| ATOM | 3609 | CA | ILE B | 596 | 71.116 | 19.084 | 79.807 | 1.00 | 34.38 | B |
| ATOM | 3610 | CB | ILE B | 596 | 70.662 | 20.479 | 79.263 | 1.00 | 35.15 | B |
| ATOM | 3611 | CG2 | ILE B | 596 | 71.746 | 21.058 | 78.357 | 1.00 | 35.63 | B |
| ATOM | 3612 | CG1 | ILE B | 596 | 69.390 | 20.334 | 78.401 | 1.00 | 36.12 | B |
| ATOM | 3613 | CD1 | ILE B | 596 | 68.175 | 19.723 | 79.073 | 1.00 | 29.57 | B |
| ATOM | 3614 | C | ILE B | 596 | 72.515 | 19.174 | 80.415 | 1.00 | 32.98 | B |
| ATOM | 3615 | O | ILE B | 596 | 72.679 | 19.563 | 81.570 | 1.00 | 30.78 | B |
| ATOM | 3616 | N | TRP B | 597 | 73.520 | 18.795 | 79.638 | 1.00 | 33.10 | B |
| ATOM | 3617 | CA | TRP B | 597 | 74.892 | 18.821 | 80.124 | 1.00 | 34.71 | B |
| ATOM | 3618 | CB | TRP B | 597 | 75.493 | 17.404 | 80.117 | 1.00 | 34.39 | B |
| ATOM | 3619 | CG | TRP B | 597 | 74.577 | 16.341 | 80.645 | 1.00 | 33.10 | B |
| ATOM | 3620 | CD2 | TRP B | 597 | 74.691 | 15.653 | 81.893 | 1.00 | 33.20 | B |
| ATOM | 3621 | CE2 | TRP B | 597 | 73.607 | 14.752 | 81.974 | 1.00 | 33.38 | B |
| ATOM | 3622 | CE3 | TRP B | 597 | 75.599 | 15.713 | 82.956 | 1.00 | 33.08 | B |
| ATOM | 3623 | CD1 | TRP B | 597 | 73.463 | 15.842 | 80.035 | 1.00 | 33.47 | B |
| ATOM | 3624 | NE1 | TRP B | 597 | 72.874 | 14.884 | 80.826 | 1.00 | 32.94 | B |
| ATOM | 3625 | CZ2 | TRP B | 597 | 73.411 | 13.912 | 83.072 | 1.00 | 33.63 | B |
| ATOM | 3626 | CZ3 | TRP B | 597 | 75.406 | 14.878 | 84.051 | 1.00 | 32.87 | B |
| ATOM | 3627 | CH2 | TRP B | 597 | 74.314 | 13.989 | 84.101 | 1.00 | 33.13 | B |
| ATOM | 3628 | C | TRP B | 597 | 75.725 | 19.726 | 79.229 | 1.00 | 35.90 | B |
| ATOM | 3629 | O | TRP B | 597 | 75.455 | 19.837 | 78.028 | 1.00 | 36.19 | B |
| ATOM | 3630 | N | ASP B | 598 | 76.731 | 20.372 | 79.817 | 1.00 | 36.61 | B |
| ATOM | 3631 | CA | ASP B | 598 | 77.631 | 21.265 | 79.082 | 1.00 | 38.19 | B |
| ATOM | 3632 | CB | ASP B | 598 | 78.156 | 22.369 | 80.018 | 1.00 | 36.98 | B |
| ATOM | 3633 | CG | ASP B | 598 | 78.999 | 23.410 | 79.291 | 1.00 | 37.20 | B |
| ATOM | 3634 | OD1 | ASP B | 598 | 79.604 | 23.089 | 78.240 | 1.00 | 36.53 | B |
| ATOM | 3635 | OD2 | ASP B | 598 | 79.059 | 24.553 | 79.786 | 1.00 | 37.52 | B |
| ATOM | 3636 | C | ASP B | 598 | 78.806 | 20.426 | 78.574 | 1.00 | 39.00 | B |
| ATOM | 3637 | O | ASP B | 598 | 79.670 | 20.050 | 79.349 | 1.00 | 39.73 | B |
| ATOM | 3638 | N | LEU B | 599 | 78.847 | 20.126 | 77.282 | 1.00 | 40.71 | B |
| ATOM | 3639 | CA | LEU B | 599 | 79.942 | 19.320 | 76.747 | 1.00 | 42.08 | B |
| ATOM | 3640 | CB | LEU B | 599 | 79.593 | 18.805 | 75.344 | 1.00 | 40.52 | B |
| ATOM | 3641 | CG | LEU B | 599 | 78.606 | 17.628 | 75.251 | 1.00 | 40.62 | B |
| ATOM | 3642 | CO1 | LEU B | 599 | 79.264 | 16.437 | 74.600 | 1.00 | 38.01 | B |
| ATOM | 3643 | CO2 | LEU B | 599 | 78.096 | 17.258 | 76.638 | 1.00 | 38.10 | B |
| ATOM | 3644 | C | LEU B | 599 | 81.295 | 20.025 | 76.721 | 1.00 | 43.84 | B |
| ATOM | 3645 | O | LEU B | 599 | 82.256 | 19.494 | 76.165 | 1.00 | 44.70 | B |
| ATOM | 3646 | N | GLU B | 600 | 81.373 | 21.216 | 77.315 | 1.00 | 44.72 | B |
| ATOM | 3647 | CA | GLU B | 600 | 82.637 | 21.941 | 77.358 | 1.00 | 45.68 | B |
| ATOM | 3648 | CB | GLU B | 600 | 82.412 | 23.413 | 77.697 | 1.00 | 48.89 | B |
| ATOM | 3649 | CG | GLU B | 600 | 83.460 | 24.367 | 77.128 | 1.00 | 52.63 | B |
| ATOM | 3650 | CD | GLU B | 600 | 84.762 | 24.375 | 77.924 | 1.00 | 56.18 | B |
| ATOM | 3651 | OE1 | GLU B | 600 | 84.739 | 24.763 | 79.120 | 1.00 | 58.19 | B |
| ATOM | 3652 | OE2 | GLU B | 600 | 85.811 | 23.997 | 77.353 | 1.00 | 57.88 | B |
| ATOM | 3653 | C | GLU B | 600 | 83.480 | 21.254 | 78.419 | 1.00 | 45.43 | B |
| ATOM | 3654 | O | GLU B | 600 | 84.683 | 21.083 | 78.242 | 1.00 | 46.22 | B |
| ATOM | 3655 | N | ASN B | 605 | 82.844 | 20.855 | 79.518 | 1.00 | 44.31 | B |
| ATOM | 3656 | CA | ASN B | 605 | 83.543 | 20.124 | 80.568 | 1.00 | 43.89 | B |
| ATOM | 3657 | CB | ASN B | 605 | 84.285 | 21.077 | 81.520 | 1.00 | 46.09 | B |
| ATOM | 3658 | CG | ASN B | 605 | 85.808 | 21.121 | 81.248 | 1.00 | 50.14 | B |
| ATOM | 3659 | OD1 | ASN B | 605 | 86.508 | 20.093 | 81.348 | 1.00 | 50.14 | B |
| ATOM | 3660 | ND2 | ASN B | 605 | 86.320 | 22.314 | 80.900 | 1.00 | 49.96 | B |
| ATOM | 3661 | C | ASN B | 605 | 82.630 | 19.186 | 81.346 | 1.00 | 41.67 | B |
| ATOM | 3662 | O | ASN B | 605 | 82.756 | 19.054 | 82.554 | 1.00 | 43.82 | B |
| ATOM | 3663 | N | GLY B | 606 | 81.718 | 18.525 | 80.638 | 1.00 | 38.96 | B |
| ATOM | 3664 | CA | GLY B | 606 | 80.804 | 17.584 | 81.268 | 1.00 | 35.37 | B |
| ATOM | 3665 | C | GLY B | 606 | 80.059 | 18.044 | 82.512 | 1.00 | 34.56 | B |
| ATOM | 3666 | O | GLY B | 606 | 79.668 | 17.231 | 83.350 | 1.00 | 32.65 | B |
| ATOM | 3667 | N | GLU B | 607 | 79.846 | 19.348 | 82.622 | 1.00 | 34.50 | B |
| ATOM | 3668 | CA | GLU B | 607 | 79.147 | 19.921 | 83.757 | 1.00 | 35.96 | B |
| ATOM | 3669 | CB | GLU B | 607 | 79.616 | 21.367 | 83.976 | 1.00 | 36.06 | B |
| ATOM | 3670 | CG | GLU B | 607 | 81.114 | 21.618 | 83.763 | 1.00 | 39.20 | B |
| ATOM | 3671 | CD | GLU B | 607 | 81.468 | 21.981 | 82.321 | 1.00 | 39.57 | B |
| ATOM | 3672 | OE1 | GLU B | 607 | 80.924 | 21.353 | 81.405 | 1.00 | 41.63 | B |
| ATOM | 3673 | OE2 | GLU B | 607 | 82.301 | 22.877 | 82.097 | 1.00 | 38.72 | B |
| ATOM | 3674 | C | GLU B | 607 | 77.622 | 19.892 | 83.504 | 1.00 | 37.13 | B |
| ATOM | 3675 | O | GLU B | 607 | 77.157 | 20.165 | 82.391 | 1.00 | 36.86 | B |
| ATOM | 3676 | N | LEU B | 608 | 76.850 | 19.539 | 84.530 | 1.00 | 37.44 | B |
| ATOM | 3677 | CA | LEU B | 608 | 75.395 | 19.513 | 84.412 | 1.00 | 37.50 | B |
| ATOM | 3678 | CB | LEU B | 608 | 74.753 | 18.822 | 85.622 | 1.00 | 37.08 | B |
| ATOM | 3679 | CG | LEU B | 608 | 73.218 | 18.925 | 85.723 | 1.00 | 37.03 | B |
| ATOM | 3680 | CD1 | LEU B | 608 | 72.593 | 18.036 | 84.658 | 1.00 | 36.12 | B |
| ATOM | 3681 | CD2 | LEU B | 608 | 72.724 | 18.533 | 87.127 | 1.00 | 34.40 | B |
| ATOM | 3682 | C | LEU B | 608 | 74.912 | 20.962 | 84.339 | 1.00 | 38.65 | B |
| ATOM | 3683 | O | LEU B | 608 | 75.442 | 21.833 | 85.030 | 1.00 | 38.26 | B |
| ATOM | 3684 | N | MSE B | 625 | 73.902 | 21.210 | 83.510 | 1.00 | 39.30 | B |
| ATOM | 3685 | CA | MSE B | 625 | 73.368 | 22.553 | 83.329 | 1.00 | 39.07 | B |
| ATOM | 3686 | CB | MSE B | 625 | 73.266 | 22.890 | 81.842 | 1.00 | 41.00 | B |
| ATOM | 3687 | CG | MSE B | 625 | 74.571 | 23.323 | 81.217 | 1.00 | 43.67 | B |
| ATOM | 3688 | SE | MSE B | 625 | 74.423 | 23.805 | 79.359 | 1.00 | 50.89 | B |
| ATOM | 3689 | CE | MSE B | 625 | 73.882 | 25.650 | 79.578 | 1.00 | 45.39 | B |
| ATOM | 3690 | C | MSE B | 625 | 72.008 | 22.737 | 83.946 | 1.00 | 38.75 | B |
| ATOM | 3691 | O | MSE B | 625 | 71.803 | 23.605 | 84.795 | 1.00 | 39.63 | B |
| ATOM | 3692 | N | TYR B | 626 | 71.068 | 21.919 | 83.495 | 1.00 | 38.88 | B |
| ATOM | 3693 | CA | TYR B | 626 | 69.700 | 22.003 | 83.975 | 1.00 | 37.07 | B |
| ATOM | 3694 | CB | TYR B | 626 | 68.861 | 22.841 | 83.016 | 1.00 | 35.69 | B |
| ATOM | 3695 | CG | TYR B | 626 | 69.457 | 24.160 | 82.618 | 1.00 | 34.35 | B |
| ATOM | 3696 | CD1 | TYR B | 626 | 69.560 | 25.199 | 83.535 | 1.00 | 33.06 | B |
| ATOM | 3697 | CE1 | TYR B | 626 | 70.013 | 26.447 | 83.150 | 1.00 | 32.14 | B |
| ATOM | 3698 | CD2 | TYR B | 626 | 69.840 | 24.397 | 81.295 | 1.00 | 34.51 | B |
| ATOM | 3699 | CE2 | TYR B | 626 | 70.296 | 25.646 | 80.896 | 1.00 | 33.07 | B |
| ATOM | 3700 | CZ | TYR B | 626 | 70.373 | 26.671 | 81.833 | 1.00 | 31.79 | B |
| ATOM | 3701 | OH | TYR B | 626 | 70.753 | 27.933 | 81.446 | 1.00 | 28.57 | B |
| ATOM | 3702 | C | TYR B | 626 | 69.067 | 20.635 | 84.040 | 1.00 | 36.34 | B |
| ATOM | 3703 | O | TYR B | 626 | 69.528 | 19.690 | 83.410 | 1.00 | 37.33 | B |
| ATOM | 3704 | N | THR B | 627 | 67.992 | 20.560 | 84.809 | 1.00 | 36.28 | B |
| ATOM | 3705 | CA | THR B | 627 | 67.203 | 19.357 | 84.958 | 1.00 | 34.33 | B |
| ATOM | 3706 | CB | THR B | 627 | 67.340 | 18.744 | 86.373 | 1.00 | 32.94 | B |
| ATOM | 3707 | OG1 | THR B | 627 | 68.684 | 18.292 | 86.562 | 1.00 | 30.38 | B |
| ATOM | 3708 | CG2 | THR B | 627 | 66.391 | 17.544 | 86.535 | 1.00 | 32.04 | B |
| ATOM | 3709 | C | THR B | 627 | 65.764 | 19.808 | 84.719 | 1.00 | 34.24 | B |
| ATOM | 3710 | O | THR B | 627 | 65.248 | 20.700 | 85.400 | 1.00 | 33.46 | B |
| ATOM | 3711 | N | LEU B | 628 | 65.137 | 19.204 | 83.722 | 1.00 | 34.53 | B |
| ATOM | 3712 | CA | LEU B | 628 | 63.772 | 19.525 | 83.355 | 1.00 | 34.30 | B |
| ATOM | 3713 | CB | LEU B | 628 | 63.636 | 19.558 | 81.835 | 1.00 | 35.54 | B |
| ATOM | 3714 | CG | LEU B | 628 | 64.814 | 20.120 | 81.056 | 1.00 | 36.04 | B |
| ATOM | 3715 | CO1 | LEU B | 628 | 64.459 | 20.071 | 79.591 | 1.00 | 37.79 | B |
| ATOM | 3716 | CD2 | LEU B | 628 | 65.137 | 21.538 | 81.500 | 1.00 | 35.35 | B |
| ATOM | 3717 | C | LEU B | 628 | 62.844 | 18.454 | 83.886 | 1.00 | 32.68 | B |
| ATOM | 3718 | O | LEU B | 628 | 63.055 | 17.261 | 83.647 | 1.00 | 33.72 | B |
| ATOM | 3719 | N | GLN B | 629 | 61.805 | 18.882 | 84.582 | 1.00 | 30.62 | B |
| ATOM | 3720 | CA | GLN B | 629 | 60.849 | 17.949 | 85.127 | 1.00 | 30.70 | B |
| ATOM | 3721 | CB | GLN B | 629 | 60.774 | 18.082 | 86.647 | 1.00 | 28.58 | B |
| ATOM | 3722 | CG | GLN B | 629 | 59.867 | 17.050 | 87.257 | 1.00 | 27.57 | B |
| ATOM | 3723 | CD | GLN B | 629 | 60.402 | 15.655 | 87.063 | 1.00 | 28.68 | B |
| ATOM | 3724 | OE1 | GLN B | 629 | 59.733 | 14.775 | 86.486 | 1.00 | 28.51 | B |
| ATOM | 3725 | NE2 | GLN B | 629 | 61.620 | 15.434 | 87.545 | 1.00 | 27.17 | B |
| ATOM | 3726 | C | GLN B | 629 | 59.473 | 18.204 | 84.544 | 1.00 | 31.36 | B |
| ATOM | 3727 | O | GLN B | 629 | 58.677 | 18.958 | 85.104 | 1.00 | 31.90 | B |
| ATOM | 3728 | N | GLY B | 630 | 59.183 | 17.567 | 83.424 | 1.00 | 31.87 | B |
| ATOM | 3729 | CA | GLY B | 630 | 57.889 | 17.767 | 82.804 | 1.00 | 33.15 | B |
| ATOM | 3730 | C | GLY B | 630 | 57.254 | 16.467 | 82.364 | 1.00 | 33.55 | B |
| ATOM | 3731 | O | GLY B | 630 | 56.511 | 16.425 | 81.385 | 1.00 | 35.60 | B |
| ATOM | 3732 | N | HIS B | 631 | 57.553 | 15.400 | 83.092 | 1.00 | 32.53 | B |
| ATOM | 3733 | CA | HIS B | 631 | 57.023 | 14.083 | 82.784 | 1.00 | 30.78 | B |
| ATOM | 3734 | CB | HIS B | 631 | 57.943 | 13.368 | 81.792 | 1.00 | 28.27 | B |
| ATOM | 3735 | CG | HIS B | 631 | 58.054 | 14.052 | 80.466 | 1.00 | 24.31 | B |
| ATOM | 3736 | CD2 | HIS B | 631 | 59.003 | 14.874 | 79.965 | 1.00 | 21.91 | B |
| ATOM | 3737 | ND1 | HIS B | 631 | 57.118 | 13.895 | 79.467 | 1.00 | 26.41 | B |
| ATOM | 3738 | CE1 | HIS B | 631 | 57.487 | 14.587 | 78.405 | 1.00 | 22.90 | B |
| ATOM | 3739 | NE2 | HIS B | 631 | 58.629 | 15.190 | 78.681 | 1.00 | 22.42 | B |
| ATOM | 3740 | C | HIS B | 631 | 56.957 | 13.302 | 84.091 | 1.00 | 30.77 | B |
| ATOM | 3741 | O | HIS B | 631 | 57.834 | 13.437 | 84.948 | 1.00 | 30.31 | B |
| ATOM | 3742 | N | THR B | 632 | 55.916 | 12.489 | 84.239 | 1.00 | 31.40 | B |
| ATOM | 3743 | CA | THR B | 632 | 55.716 | 11.690 | 85.447 | 1.00 | 32.08 | B |
| ATOM | 3744 | CB | THR B | 632 | 54.281 | 11.742 | 85.914 | 1.00 | 32.58 | B |
| ATOM | 3745 | OG1 | THR B | 632 | 53.446 | 11.255 | 84.860 | 1.00 | 33.44 | B |
| ATOM | 3746 | CG2 | THR B | 632 | 53.877 | 13.151 | 86.274 | 1.00 | 31.80 | B |
| ATOM | 3747 | C | THR B | 632 | 55.990 | 10.233 | 85.152 | 1.00 | 32.55 | B |
| ATOM | 3748 | O | THR B | 632 | 55.790 | 9.367 | 86.000 | 1.00 | 33.19 | B |
| ATOM | 3749 | N | ALA B | 633 | 56.442 | 9.963 | 83.941 | 1.00 | 32.22 | B |
| ATOM | 3750 | CA | ALA B | 633 | 56.721 | 8.604 | 83.537 | 1.00 | 32.78 | B |
| ATOM | 3751 | CB | ALA B | 633 | 55.596 | 8.106 | 82.627 | 1.00 | 33.46 | B |
| ATOM | 3752 | C | ALA B | 633 | 58.048 | 8.649 | 82.786 | 1.00 | 33.18 | B |
| ATOM | 3753 | O | ALA B | 633 | 58.570 | 9.730 | 82.526 | 1.00 | 34.34 | B |
| ATOM | 3754 | N | LEU B | 634 | 58.606 | 7.502 | 82.419 | 1.00 | 32.23 | B |
| ATOM | 3755 | CA | LEU B | 634 | 59.881 | 7.559 | 81.718 | 1.00 | 31.56 | B |
| ATOM | 3756 | CE | LEU B | 634 | 60.531 | 6.176 | 81.610 | 1.00 | 32.84 | B |
| ATOM | 3757 | CG | LEU B | 634 | 59.855 | 5.099 | 80.783 | 1.00 | 33.81 | B |
| ATOM | 3758 | CD1 | LEU B | 634 | 58.372 | 5.151 | 81.095 | 1.00 | 37.82 | B |
| ATOM | 3759 | CD2 | LEU B | 634 | 60.122 | 5.300 | 79.304 | 1.00 | 32.77 | B |
| ATOM | 3760 | C | LEU B | 634 | 59.787 | 8.214 | 80.353 | 1.00 | 29.60 | B |
| ATOM | 3761 | O | LEU B | 634 | 58.807 | 8.072 | 79.620 | 1.00 | 27.99 | B |
| ATOM | 3762 | N | VAL B | 635 | 60.835 | 8.961 | 80.045 | 1.00 | 27.96 | B |
| ATOM | 3763 | CA | VAL B | 635 | 60.945 | 9.688 | 78.803 | 1.00 | 25.69 | B |
| ATOM | 3764 | CB | VAL B | 635 | 61.584 | 11.046 | 79.038 | 1.00 | 27.20 | B |
| ATOM | 3765 | CG1 | VAL B | 635 | 61.377 | 11.926 | 77.810 | 1.00 | 28.07 | B |
| ATOM | 3766 | CG2 | VAL B | 635 | 61.000 | 11.679 | 80.302 | 1.00 | 25.75 | B |
| ATOM | 3767 | C | VAL B | 635 | 61.827 | 8.948 | 77.840 | 1.00 | 23.61 | B |
| ATOM | 3768 | O | VAL B | 635 | 63.046 | 9.033 | 77.942 | 1.00 | 23.52 | B |
| ATOM | 3769 | N | GLY B | 636 | 61.219 | 8.248 | 76.891 | 1.00 | 22.88 | B |
| ATOM | 3770 | CA | GLY B | 636 | 61.998 | 7.488 | 75.928 | 1.00 | 22.35 | B |
| ATOM | 3771 | C | GLY B | 636 | 62.224 | 8.089 | 74.550 | 1.00 | 23.37 | B |
| ATOM | 3772 | O | GLY B | 636 | 62.907 | 7.480 | 73.736 | 1.00 | 24.09 | B |
| ATOM | 3773 | N | LEU B | 637 | 61.656 | 9.262 | 74.277 | 1.00 | 23.96 | B |
| ATOM | 3774 | CA | LEU B | 637 | 61.827 | 9.904 | 72.981 | 1.00 | 23.22 | B |
| ATOM | 3775 | CB | LEU B | 637 | 60.503 | 9.934 | 72.228 | 1.00 | 22.08 | B |
| ATOM | 3776 | CG | LEU B | 637 | 59.913 | 8.534 | 72.102 | 1.00 | 21.07 | B |
| ATOM | 3777 | CD1 | LEU B | 637 | 58.494 | 8.554 | 71.516 | 1.00 | 18.52 | B |
| ATOM | 3778 | CD2 | LEU B | 637 | 60.863 | 7.724 | 71.261 | 1.00 | 18.91 | B |
| ATOM | 3779 | C | LEU B | 637 | 62.328 | 11.317 | 73.164 | 1.00 | 25.24 | B |
| ATOM | 3780 | O | LEU B | 637 | 62.058 | 11.945 | 74.197 | 1.00 | 25.36 | B |
| ATOM | 3781 | N | LEU B | 638 | 63.048 | 11.797 | 72.142 | 1.00 | 26.06 | B |
| ATOM | 3782 | CA | LEU B | 638 | 63.635 | 13.147 | 72.077 | 1.00 | 25.52 | B |
| ATOM | 3783 | CB | LEU B | 638 | 64.907 | 13.258 | 72.926 | 1.00 | 25.76 | B |
| ATOM | 3784 | CG | LEU B | 638 | 64.857 | 13.321 | 74.453 | 1.00 | 26.07 | B |
| ATOM | 3785 | CD1 | LEU B | 638 | 66.285 | 13.384 | 74.983 | 1.00 | 27.82 | B |
| ATOM | 3786 | CD2 | LEU B | 638 | 64.073 | 14.535 | 74.897 | 1.00 | 25.88 | B |
| ATOM | 3787 | C | LEU B | 638 | 64.022 | 13.494 | 70.653 | 1.00 | 25.17 | B |
| ATOM | 3788 | O | LEU B | 638 | 64.529 | 12.644 | 69.919 | 1.00 | 25.44 | B |
| ATOM | 3789 | N | ARG B | 639 | 63.771 | 14.744 | 70.268 | 1.00 | 25.50 | B |
| ATOM | 3790 | CA | ARG B | 639 | 64.126 | 15.260 | 68.942 | 1.00 | 25.66 | B |
| ATOM | 3791 | CB | ARG B | 639 | 63.033 | 14.936 | 67.901 | 1.00 | 25.21 | B |
| ATOM | 3792 | CG | ARG B | 639 | 62.720 | 13.427 | 67.878 | 1.00 | 28.60 | B |
| ATOM | 3793 | CD | ARG B | 639 | 62.173 | 12.811 | 66.583 | 1.00 | 28.49 | B |
| ATOM | 3794 | NE | ARG B | 639 | 63.252 | 12.293 | 65.730 | 1.00 | 30.47 | B |
| ATOM | 3795 | CZ | ARG B | 639 | 63.131 | 11.295 | 64.851 | 1.00 | 30.02 | B |
| ATOM | 3796 | NH1 | ARG B | 639 | 61.970 | 10.668 | 64.684 | 1.00 | 27.71 | B |
| ATOM | 3797 | NH2 | ARG B | 639 | 64.183 | 10.926 | 64.123 | 1.00 | 32.50 | B |
| ATOM | 3798 | C | ARG B | 639 | 64.379 | 16.760 | 69.078 | 1.00 | 25.33 | B |
| ATOM | 3799 | O | ARG B | 639 | 63.738 | 17.442 | 69.877 | 1.00 | 24.41 | B |
| ATOM | 3800 | N | LEU B | 640 | 65.355 | 17.258 | 68.332 | 1.00 | 26.36 | B |
| ATOM | 3801 | CA | LEU B | 640 | 65.707 | 18.661 | 68.408 | 1.00 | 27.00 | B |
| ATOM | 3802 | CB | LEU B | 640 | 67.214 | 18.808 | 68.563 | 1.00 | 28.00 | B |
| ATOM | 3803 | CG | LEU B | 640 | 67.688 | 19.504 | 69.834 | 1.00 | 27.72 | B |
| ATOM | 3804 | CD1 | LEU B | 640 | 69.188 | 19.757 | 69.737 | 1.00 | 25.31 | B |
| ATOM | 3805 | CD2 | LEU B | 640 | 66.914 | 20.802 | 70.019 | 1.00 | 25.63 | B |
| ATOM | 3806 | C | LEU B | 640 | 65.263 | 19.432 | 67.189 | 1.00 | 27.04 | B |
| ATOM | 3807 | O | LEU B | 640 | 65.759 | 19.210 | 66.097 | 1.00 | 27.98 | B |
| ATOM | 3808 | N | SER B | 641 | 64.310 | 20.329 | 67.373 | 1.00 | 29.24 | B |
| ATOM | 3809 | CA | SER B | 641 | 63.828 | 21.138 | 66.271 | 1.00 | 30.57 | B |
| ATOM | 3810 | CB | SER B | 641 | 62.359 | 21.509 | 66.504 | 1.00 | 31.54 | B |
| ATOM | 3811 | OG | SER B | 641 | 62.025 | 22.763 | 65.944 | 1.00 | 30.17 | B |
| ATOM | 3812 | C | SER B | 641 | 64.718 | 22.366 | 66.277 | 1.00 | 31.05 | B |
| ATOM | 3813 | O | SER B | 641 | 65.499 | 22.560 | 67.198 | 1.00 | 32.13 | B |
| ATOM | 3814 | N | ASP B | 642 | 64.628 | 23.194 | 65.254 | 1.00 | 32.29 | B |
| ATOM | 3815 | CA | ASP B | 642 | 65.472 | 24.373 | 65.210 | 1.00 | 32.45 | B |
| ATOM | 3816 | CB | ASP B | 642 | 65.293 | 25.059 | 63.870 | 1.00 | 36.58 | B |
| ATOM | 3817 | CG | ASP B | 642 | 66.575 | 25.652 | 63.360 | 1.00 | 40.19 | B |
| ATOM | 3818 | OD1 | ASP B | 642 | 67.336 | 24.914 | 62.681 | 1.00 | 41.18 | B |
| ATOM | 3819 | OD2 | ASP B | 642 | 66.816 | 26.848 | 63.661 | 1.00 | 42.03 | B |
| ATOM | 3820 | C | ASP B | 642 | 65.140 | 25.348 | 66.344 | 1.00 | 31.30 | B |
| ATOM | 3821 | O | ASP B | 642 | 65.996 | 26.095 | 66.809 | 1.00 | 28.33 | B |
| ATOM | 3822 | N | LYS B | 643 | 63.876 | 25.332 | 66.759 | 1.00 | 30.65 | B |
| ATOM | 3823 | CA | LYS B | 643 | 63.373 | 26.190 | 67.818 | 1.00 | 28.54 | B |
| ATOM | 3824 | CB | LYS B | 643 | 62.069 | 26.888 | 67.407 | 1.00 | 28.11 | B |
| ATOM | 3825 | CG | LYS B | 643 | 62.141 | 27.807 | 66.210 | 1.00 | 30.60 | B |
| ATOM | 3826 | CD | LYS B | 643 | 63.196 | 28.902 | 66.364 | 1.00 | 32.54 | B |
| ATOM | 3827 | CE | LYS B | 643 | 63.332 | 29.736 | 65.070 | 1.00 | 33.58 | B |
| ATOM | 3828 | NZ | LYS B | 643 | 63.760 | 28.917 | 63.873 | 1.00 | 33.86 | B |
| ATOM | 3829 | C | LYS B | 643 | 63.047 | 25.391 | 69.077 | 1.00 | 28.15 | B |
| ATOM | 3830 | O | LYS B | 643 | 63.019 | 25.951 | 70.163 | 1.00 | 28.92 | B |
| ATOM | 3831 | N | PHE B | 644 | 62.787 | 24.094 | 68.957 | 1.00 | 26.09 | B |
| ATOM | 3832 | CA | PHE B | 644 | 62.413 | 23.353 | 70.152 | 1.00 | 24.49 | B |
| ATOM | 3833 | CB | PHE B | 644 | 60.931 | 22.958 | 70.115 | 1.00 | 21.46 | B |
| ATOM | 3834 | CG | PHE B | 644 | 60.015 | 24.023 | 69.613 | 1.00 | 20.17 | B |
| ATOM | 3835 | CD1 | PHE B | 644 | 59.720 | 24.125 | 68.258 | 1.00 | 22.46 | B |
| ATOM | 3836 | CD2 | PHE B | 644 | 59.404 | 24.896 | 70.493 | 1.00 | 20.58 | B |
| ATOM | 3837 | CE1 | PHE B | 644 | 58.816 | 25.079 | 67.783 | 1.00 | 20.21 | B |
| ATOM | 3838 | CE2 | PHE B | 644 | 58.501 | 25.855 | 70.035 | 1.00 | 20.45 | B |
| ATOM | 3839 | CZ | PHE B | 644 | 58.207 | 25.942 | 68.674 | 1.00 | 20.84 | B |
| ATOM | 3840 | C | PHE B | 644 | 63.186 | 22.087 | 70.457 | 1.00 | 25.20 | B |
| ATOM | 3841 | O | PHE B | 644 | 63.886 | 21.534 | 69.613 | 1.00 | 24.80 | B |
| ATOM | 3842 | N | LEU B | 645 | 63.054 | 21.649 | 71.701 | 1.00 | 25.69 | B |
| ATOM | 3843 | CA | LEU B | 645 | 63.630 | 20.396 | 72.151 | 1.00 | 25.88 | B |
| ATOM | 3844 | CB | LEU B | 645 | 64.547 | 20.586 | 73.359 | 1.00 | 26.05 | B |
| ATOM | 3845 | CG | LEU B | 645 | 64.777 | 19.327 | 74.233 | 1.00 | 27.77 | B |
| ATOM | 3846 | CD1 | LEU B | 645 | 66.044 | 18.602 | 73.852 | 1.00 | 28.34 | B |
| ATOM | 3847 | CD2 | LEU B | 645 | 64.864 | 19.739 | 75.692 | 1.00 | 29.93 | B |
| ATOM | 3848 | C | LEU B | 645 | 62.349 | 19.704 | 72.590 | 1.00 | 26.32 | B |
| ATOM | 3849 | O | LEU B | 645 | 61.743 | 20.096 | 73.591 | 1.00 | 27.97 | B |
| ATOM | 3850 | N | VAL B | 646 | 61.910 | 18.706 | 71.834 | 1.00 | 25.54 | B |
| ATOM | 3851 | CA | VAL B | 646 | 60.688 | 18.005 | 72.191 | 1.00 | 25.91 | B |
| ATOM | 3852 | CB | VAL B | 646 | 59.892 | 17.651 | 70.918 | 1.00 | 25.69 | B |
| ATOM | 3853 | CG1 | VAL B | 646 | 58.554 | 17.036 | 71.293 | 1.00 | 26.37 | B |
| ATOM | 3854 | CG2 | VAL B | 646 | 59.681 | 18.909 | 70.069 | 1.00 | 23.92 | B |
| ATOM | 3855 | C | VAL B | 646 | 61.003 | 16.734 | 72.986 | 1.00 | 26.60 | B |
| ATOM | 3856 | O | VAL B | 646 | 62.051 | 16.130 | 72.780 | 1.00 | 27.76 | B |
| ATOM | 3857 | N | SER B | 647 | 60.121 | 16.358 | 73.916 | 1.00 | 27.17 | B |
| ATOM | 3858 | CA | SER B | 647 | 60.281 | 15.130 | 74.711 | 1.00 | 27.29 | B |
| ATOM | 3859 | CB | SER B | 647 | 60.956 | 15.417 | 76.054 | 1.00 | 27.08 | B |
| ATOM | 3860 | OG | SER B | 647 | 60.210 | 16.325 | 76.850 | 1.00 | 27.93 | B |
| ATOM | 3861 | C | SER B | 647 | 58.905 | 14.512 | 74.942 | 1.00 | 28.49 | B |
| ATOM | 3862 | O | SER B | 647 | 57.943 | 15.227 | 75.219 | 1.00 | 29.23 | B |
| ATOM | 3863 | N | ALA B | 648 | 58.819 | 13.189 | 74.800 | 1.00 | 28.67 | B |
| ATOM | 3864 | CA | ALA B | 648 | 57.568 | 12.454 | 74.976 | 1.00 | 28.62 | B |
| ATOM | 3865 | CB | ALA B | 648 | 57.093 | 11.906 | 73.630 | 1.00 | 28.17 | B |
| ATOM | 3866 | C | ALA B | 648 | 57.760 | 11.306 | 75.972 | 1.00 | 30.51 | B |
| ATOM | 3867 | O | ALA B | 648 | 58.756 | 10.577 | 75.898 | 1.00 | 31.42 | B |
| ATOM | 3868 | N | ALA B | 649 | 56.807 | 11.146 | 76.894 | 1.00 | 30.32 | B |
| ATOM | 3869 | CA | ALA B | 649 | 56.883 | 10.098 | 77.911 | 1.00 | 30.33 | B |
| ATOM | 3870 | CB | ALA B | 649 | 56.720 | 10.700 | 79.305 | 1.00 | 29.35 | B |
| ATOM | 3871 | C | ALA B | 649 | 55.863 | 8.988 | 77.711 | 1.00 | 31.09 | B |
| ATOM | 3872 | O | ALA B | 649 | 54.995 | 9.080 | 76.850 | 1.00 | 30.54 | B |
| ATOM | 3873 | N | ALA B | 650 | 55.981 | 7.941 | 78.530 | 1.00 | 32.95 | B |
| ATOM | 3874 | CA | ALA B | 650 | 55.103 | 6.768 | 78.483 | 1.00 | 32.69 | B |
| ATOM | 3875 | CB | ALA B | 650 | 55.783 | 5.597 | 79.194 | 1.00 | 30.66 | B |
| ATOM | 3876 | C | ALA B | 650 | 53.733 | 7.019 | 79.098 | 1.00 | 33.02 | B |
| ATOM | 3877 | O | ALA B | 650 | 52.941 | 6.091 | 79.251 | 1.00 | 32.69 | B |
| ATOM | 3878 | N | ASP B | 651 | 53.471 | 8.270 | 79.463 | 1.00 | 34.34 | B |
| ATOM | 3879 | CA | ASP B | 651 | 52.200 | 8.661 | 80.063 | 1.00 | 35.15 | B |
| ATOM | 3880 | CB | ASP B | 651 | 52.456 | 9.528 | 81.287 | 1.00 | 36.84 | B |
| ATOM | 3881 | CG | ASP B | 651 | 53.402 | 10.663 | 80.993 | 1.00 | 40.07 | B |
| ATOM | 3882 | OD1 | ASP B | 651 | 53.652 | 10.935 | 79.800 | 1.00 | 40.99 | B |
| ATOM | 3883 | OD2 | ASP B | 651 | 53.895 | 11.299 | 81.950 | 1.00 | 43.26 | B |
| ATOM | 3884 | C | ASP B | 651 | 51.347 | 9.441 | 79.068 | 1.00 | 35.49 | B |
| ATOM | 3885 | O | ASP B | 651 | 50.282 | 9.948 | 79.418 | 1.00 | 35.89 | B |
| ATOM | 3886 | N | GLY B | 652 | 51.833 | 9.542 | 77.834 | 1.00 | 34.99 | B |
| ATOM | 3887 | CA | GLY B | 652 | 51.110 | 10.260 | 76.802 | 1.00 | 34.08 | B |
| ATOM | 3888 | C | GLY B | 652 | 51.434 | 11.738 | 76.730 | 1.00 | 34.36 | B |
| ATOM | 3889 | O | GLY B | 652 | 50.877 | 12.449 | 75.891 | 1.00 | 36.08 | B |
| ATOM | 3890 | N | SER B | 653 | 52.336 | 12.198 | 77.595 | 1.00 | 32.03 | B |
| ATOM | 3891 | CA | SER B | 653 | 52.720 | 13.605 | 77.635 | 1.00 | 30.49 | B |
| ATOM | 3892 | CB | SER B | 653 | 53.176 | 13.995 | 79.050 | 1.00 | 31.53 | B |
| ATOM | 3893 | OG | SER B | 653 | 54.387 | 13.358 | 79.428 | 1.00 | 31.63 | B |
| ATOM | 3894 | C | SER B | 653 | 53.810 | 13.962 | 76.633 | 1.00 | 29.75 | B |
| ATOM | 3895 | O | SER B | 653 | 54.702 | 13.165 | 76.361 | 1.00 | 28.99 | B |
| ATOM | 3896 | N | ILE B | 654 | 53.733 | 15.175 | 76.097 | 1.00 | 30.15 | B |
| ATOM | 3897 | CA | ILE B | 654 | 54.703 | 15.655 | 75.115 | 1.00 | 30.00 | B |
| ATOM | 3898 | CB | ILE B | 654 | 54.083 | 15.708 | 73.706 | 1.00 | 29.09 | B |
| ATOM | 3899 | CG2 | ILE B | 654 | 55.115 | 16.144 | 72.686 | 1.00 | 28.47 | B |
| ATOM | 3900 | CG1 | ILE B | 654 | 53.548 | 14.336 | 73.318 | 1.00 | 28.15 | B |
| ATOM | 3901 | CD1 | ILE B | 654 | 52.204 | 14.400 | 72.675 | 1.00 | 30.38 | B |
| ATOM | 3902 | C | ILE B | 654 | 55.109 | 17.061 | 75.508 | 1.00 | 30.87 | B |
| ATOM | 3903 | O | ILE B | 654 | 54.285 | 17.974 | 75.484 | 1.00 | 32.52 | B |
| ATOM | 3904 | N | ARG B | 655 | 56.371 | 17.241 | 75.878 | 1.00 | 30.22 | B |
| ATOM | 3905 | CA | ARG B | 655 | 56.835 | 18.570 | 76.271 | 1.00 | 29.48 | B |
| ATOM | 3906 | CB | ARG B | 655 | 57.614 | 18.492 | 77.589 | 1.00 | 31.26 | B |
| ATOM | 3907 | CG | ARG B | 655 | 56.773 | 18.219 | 78.827 | 1.00 | 34.00 | B |
| ATOM | 3908 | CD | ARG B | 655 | 56.140 | 19.500 | 79.287 | 1.00 | 40.70 | B |
| ATOM | 3909 | NE | ARG B | 655 | 55.601 | 19.484 | 80.657 | 1.00 | 45.23 | B |
| ATOM | 3910 | CZ | ARG B | 655 | 54.582 | 18.735 | 81.075 | 1.00 | 48.41 | B |
| ATOM | 3911 | NH1 | ARG B | 655 | 53.979 | 17.909 | 80.234 | 1.00 | 49.81 | B |
| ATOM | 3912 | NH2 | ARG B | 655 | 54.137 | 18.847 | 82.324 | 1.00 | 50.20 | B |
| ATOM | 3913 | C | ARG B | 655 | 57.712 | 19.195 | 75.187 | 1.00 | 28.14 | B |
| ATOM | 3914 | O | ARG B | 655 | 58.462 | 18.501 | 74.500 | 1.00 | 27.90 | B |
| ATOM | 3915 | N | GLY B | 656 | 57.582 | 20.507 | 75.033 | 1.00 | 26.00 | B |
| ATOM | 3916 | CA | GLY B | 656 | 58.365 | 21.237 | 74.065 | 1.00 | 24.28 | B |
| ATOM | 3917 | C | GLY B | 656 | 59.047 | 22.348 | 74.830 | 1.00 | 24.73 | B |
| ATOM | 3918 | O | GLY B | 656 | 58.384 | 23.228 | 75.386 | 1.00 | 26.11 | B |
| ATOM | 3919 | N | TRP B | 657 | 60.373 | 22.296 | 74.883 | 1.00 | 24.11 | B |
| ATOM | 3920 | CA | TRP B | 657 | 61.163 | 23.294 | 75.601 | 1.00 | 23.50 | B |
| ATOM | 3921 | CB | TRP B | 657 | 62.139 | 22.602 | 76.550 | 1.00 | 22.75 | B |
| ATOM | 3922 | CG | TRP B | 657 | 61.579 | 21.381 | 77.189 | 1.00 | 21.95 | B |
| ATOM | 3923 | CD2 | TRP B | 657 | 61.213 | 21.230 | 78.562 | 1.00 | 19.79 | B |
| ATOM | 3924 | CE2 | TRP B | 657 | 60.639 | 19.948 | 78.698 | 1.00 | 20.88 | B |
| ATOM | 3925 | CE3 | TRP B | 657 | 61.320 | 22.048 | 79.691 | 1.00 | 19.86 | B |
| ATOM | 3926 | CD1 | TRP B | 657 | 61.231 | 20.213 | 76.566 | 1.00 | 20.79 | B |
| ATOM | 3927 | NE1 | TRP B | 657 | 60.657 | 19.350 | 77.469 | 1.00 | 20.21 | B |
| ATOM | 3928 | CZ2 | TRP B | 657 | 60.160 | 19.478 | 79.926 | 1.00 | 21.49 | B |
| ATOM | 3929 | CZ3 | TRP B | 657 | 60.846 | 21.570 | 80.915 | 1.00 | 20.44 | B |
| ATOM | 3930 | CR2 | TRP B | 657 | 60.278 | 20.301 | 81.019 | 1.00 | 20.36 | B |
| ATOM | 3931 | C | TRP B | 657 | 61.966 | 24.122 | 74.610 | 1.00 | 24.96 | B |
| ATOM | 3932 | O | TRP B | 657 | 62.262 | 23.673 | 73.498 | 1.00 | 24.34 | B |
| ATOM | 3933 | N | ASP B | 658 | 62.331 | 25.331 | 75.014 | 1.00 | 26.03 | B |
| ATOM | 3934 | CA | ASP B | 658 | 63.126 | 26.181 | 74.137 | 1.00 | 28.03 | B |
| ATOM | 3935 | CB | ASP B | 658 | 63.307 | 27.565 | 74.741 | 1.00 | 28.12 | B |
| ATOM | 3936 | CG | ASP B | 658 | 64.031 | 28.494 | 73.805 | 1.00 | 29.75 | B |
| ATOM | 3937 | OD1 | ASP B | 658 | 65.264 | 28.356 | 73.641 | 1.00 | 28.58 | B |
| ATOM | 3938 | OD2 | ASP B | 658 | 63.352 | 29.351 | 73.209 | 1.00 | 31.24 | B |
| ATOM | 3939 | C | ASP B | 658 | 64.498 | 25.538 | 73.961 | 1.00 | 28.55 | B |
| ATOM | 3940 | O | ASP B | 658 | 65.168 | 25.253 | 74.948 | 1.00 | 31.11 | B |
| ATOM | 3941 | N | ALA B | 659 | 64.924 | 25.334 | 72.719 | 1.00 | 27.30 | B |
| ATOM | 3942 | CA | ALA B | 659 | 66.209 | 24.689 | 72.443 | 1.00 | 28.28 | B |
| ATOM | 3943 | CB | ALA B | 659 | 66.413 | 24.549 | 70.945 | 1.00 | 26.59 | B |
| ATOM | 3944 | C | ALA B | 659 | 67.430 | 25.362 | 73.047 | 1.00 | 29.08 | B |
| ATOM | 3945 | O | ALA B | 659 | 68.518 | 24.784 | 73.067 | 1.00 | 30.37 | B |
| ATOM | 3946 | N | ASN B | 660 | 67.272 | 26.577 | 73.544 | 1.00 | 29.04 | B |
| ATOM | 3947 | CA | ASN B | 660 | 68.419 | 27.256 | 74.090 | 1.00 | 29.15 | B |
| ATOM | 3948 | CB | ASN B | 660 | 68.618 | 28.573 | 73.361 | 1.00 | 32.02 | B |
| ATOM | 3949 | CG | ASN B | 660 | 69.953 | 29.185 | 73.646 | 1.00 | 36.15 | B |
| ATOM | 3950 | OD1 | ASN B | 660 | 70.999 | 28.600 | 73.342 | 1.00 | 39.04 | B |
| ATOM | 3951 | ND2 | ASN B | 660 | 69.939 | 30.371 | 74.238 | 1.00 | 38.31 | B |
| ATOM | 3952 | C | ASN B | 660 | 68.334 | 27.474 | 75.582 | 1.00 | 28.96 | B |
| ATOM | 3953 | O | ASN B | 660 | 69.262 | 27.138 | 76.301 | 1.00 | 30.76 | B |
| ATOM | 3954 | N | ASP B | 661 | 67.225 | 28.024 | 76.059 | 1.00 | 28.20 | B |
| ATOM | 3955 | CA | ASP B | 661 | 67.073 | 28.249 | 77.485 | 1.00 | 26.87 | B |
| ATOM | 3956 | CB | ASP B | 661 | 66.553 | 29.663 | 77.745 | 1.00 | 29.19 | B |
| ATOM | 3957 | CG | ASP B | 661 | 65.311 | 29.984 | 76.942 | 1.00 | 30.92 | B |
| ATOM | 3958 | OD1 | ASP B | 661 | 65.314 | 31.019 | 76.232 | 1.00 | 32.39 | B |
| ATOM | 3959 | OD2 | ASP B | 661 | 64.334 | 29.209 | 77.023 | 1.00 | 30.85 | B |
| ATOM | 3960 | C | ASP B | 661 | 66.147 | 27.217 | 78.129 | 1.00 | 26.15 | B |
| ATOM | 3961 | O | ASP B | 661 | 66.041 | 27.165 | 79.346 | 1.00 | 28.27 | B |
| ATOM | 3962 | N | TYR B | 662 | 65.474 | 26.409 | 77.317 | 1.00 | 22.94 | B |
| ATOM | 3963 | CA | TYR B | 662 | 64.590 | 25.365 | 77.819 | 1.00 | 21.57 | B |
| ATOM | 3964 | CB | TYR B | 662 | 65.407 | 24.340 | 78.623 | 1.00 | 20.55 | B |
| ATOM | 3965 | CG | TYR B | 662 | 66.647 | 23.879 | 77.871 | 1.00 | 21.52 | B |
| ATOM | 3966 | CD1 | TYR B | 662 | 67.867 | 24.533 | 78.019 | 1.00 | 19.76 | B |
| ATOM | 3967 | CE1 | TYR B | 662 | 68.960 | 24.193 | 77.231 | 1.00 | 18.58 | B |
| ATOM | 3968 | CD2 | TYR B | 662 | 66.567 | 22.861 | 76.919 | 1.00 | 20.80 | B |
| ATOM | 3969 | CE2 | TYR B | 662 | 67.659 | 22.519 | 76.126 | 1.00 | 18.36 | B |
| ATOM | 3970 | CZ | TYR B | 662 | 68.851 | 23.192 | 76.287 | 1.00 | 18.65 | B |
| ATOM | 3971 | OH | TYR B | 662 | 69.935 | 22.893 | 75.493 | 1.00 | 18.77 | B |
| ATOM | 3972 | C | TYR B | 662 | 63.346 | 25.808 | 78.604 | 1.00 | 21.16 | B |
| ATOM | 3973 | O | TYR B | 662 | 62.835 | 25.068 | 79.443 | 1.00 | 20.62 | B |
| ATOM | 3974 | N | SER B | 663 | 62.864 | 27.015 | 78.327 | 1.00 | 21.52 | B |
| ATOM | 3975 | CA | SER B | 663 | 61.642 | 27.500 | 78.954 | 1.00 | 21.24 | B |
| ATOM | 3976 | CB | SER B | 663 | 61.458 | 28.993 | 78.699 | 1.00 | 19.14 | B |
| ATOM | 3977 | OG | SER B | 663 | 61.441 | 29.273 | 77.312 | 1.00 | 19.19 | B |
| ATOM | 3978 | C | SER B | 663 | 60.496 | 26.728 | 78.294 | 1.00 | 21.60 | B |
| ATOM | 3979 | O | SER B | 663 | 60.585 | 26.352 | 77.123 | 1.00 | 20.87 | B |
| ATOM | 3980 | N | ARG B | 664 | 59.436 | 26.489 | 79.057 | 1.00 | 22.75 | B |
| ATOM | 3981 | CA | ARG B | 664 | 58.267 | 25.755 | 78.592 | 1.00 | 23.75 | B |
| ATOM | 3982 | CB | ARG B | 664 | 57.317 | 25.544 | 79.764 | 1.00 | 26.18 | B |
| ATOM | 3983 | CG | ARG B | 664 | 58.032 | 24.928 | 80.948 | 1.00 | 33.56 | B |
| ATOM | 3984 | CD | ARG B | 664 | 57.130 | 24.061 | 81.812 | 1.00 | 39.29 | B |
| ATOM | 3985 | NE | ARG B | 664 | 57.925 | 23.331 | 82.801 | 1.00 | 43.83 | B |
| ATOM | 3986 | CZ | ARG B | 664 | 57.446 | 22.404 | 83.625 | 1.00 | 47.40 | B |
| ATOM | 3987 | NH1 | ARG B | 664 | 56.158 | 22.076 | 83.594 | 1.00 | 48.81 | B |
| ATOM | 3988 | NH2 | ARG B | 664 | 58.265 | 21.796 | 84.474 | 1.00 | 48.40 | B |
| ATOM | 3989 | C | ARG B | 664 | 57.540 | 26.436 | 77.433 | 1.00 | 23.79 | B |
| ATOM | 3990 | O | ARG B | 664 | 56.829 | 27.420 | 77.632 | 1.00 | 23.72 | B |
| ATOM | 3991 | N | LYS B | 665 | 57.702 | 25.886 | 76.228 | 1.00 | 23.87 | B |
| ATOM | 3992 | CA | LYS B | 665 | 57.091 | 26.453 | 75.021 | 1.00 | 24.18 | B |
| ATOM | 3993 | CB | LYS B | 665 | 58.099 | 26.400 | 73.869 | 1.00 | 25.26 | B |
| ATOM | 3994 | CG | LYS B | 665 | 59.088 | 27.551 | 73.869 | 1.00 | 26.13 | B |
| ATOM | 3995 | CD | LYS B | 665 | 58.423 | 28.779 | 73.292 | 1.00 | 31.91 | B |
| ATOM | 3996 | CE | LYS B | 665 | 59.389 | 29.964 | 73.126 | 1.00 | 34.51 | B |
| ATOM | 3997 | NZ | LYS B | 665 | 59.804 | 30.608 | 74.426 | 1.00 | 40.98 | B |
| ATOM | 3998 | C | LYS B | 665 | 55.760 | 25.844 | 74.588 | 1.00 | 22.25 | B |
| ATOM | 3999 | O | LYS B | 665 | 54.943 | 26.514 | 73.956 | 1.00 | 21.84 | B |
| ATOM | 4000 | N | PHE B | 666 | 55.555 | 24.574 | 74.917 | 1.00 | 22.07 | B |
| ATOM | 4001 | CA | PHE B | 666 | 54.308 | 23.875 | 74.604 | 1.00 | 22.00 | B |
| ATOM | 4002 | CB | PHE B | 666 | 54.109 | 23.776 | 73.076 | 1.00 | 21.53 | B |
| ATOM | 4003 | CG | PHE B | 666 | 55.017 | 22.786 | 72.373 | 1.00 | 21.45 | B |
| ATOM | 4004 | CD1 | PHE B | 666 | 54.744 | 21.420 | 72.408 | 1.00 | 21.12 | B |
| ATOM | 4005 | CD2 | PHE B | 666 | 56.134 | 23.218 | 71.664 | 1.00 | 20.63 | B |
| ATOM | 4006 | CE1 | PHE B | 666 | 55.562 | 20.506 | 71.758 | 1.00 | 17.08 | B |
| ATOM | 4007 | CE2 | PHE B | 666 | 56.951 | 22.303 | 71.015 | 1.00 | 19.89 | B |
| ATOM | 4008 | CZ | PHE B | 666 | 56.659 | 20.947 | 71.066 | 1.00 | 17.19 | B |
| ATOM | 4009 | C | PHE B | 666 | 54.272 | 22.494 | 75.288 | 1.00 | 22.61 | B |
| ATOM | 4010 | O | PHE B | 666 | 55.323 | 21.938 | 75.618 | 1.00 | 24.01 | B |
| ATOM | 4011 | N | SER B | 667 | 53.068 | 21.973 | 75.538 | 1.00 | 22.74 | B |
| ATOM | 4012 | CA | SER B | 667 | 52.891 | 20.674 | 76.200 | 1.00 | 23.29 | B |
| ATOM | 4013 | CB | SER B | 667 | 52.827 | 20.831 | 77.717 | 1.00 | 22.23 | B |
| ATOM | 4014 | OG | SER B | 667 | 51.487 | 20.937 | 78.145 | 1.00 | 23.02 | B |
| ATOM | 4015 | C | SER B | 667 | 51.596 | 20.014 | 75.737 | 1.00 | 24.06 | B |
| ATOM | 4016 | O | SER B | 667 | 50.525 | 20.635 | 75.772 | 1.00 | 23.18 | B |
| ATOM | 4017 | N | TYR B | 668 | 51.703 | 18.751 | 75.321 | 1.00 | 24.19 | B |
| ATOM | 4018 | CA | TYR B | 668 | 50.554 | 17.999 | 74.834 | 1.00 | 24.45 | B |
| ATOM | 4019 | CB | TYR B | 668 | 50.633 | 17.810 | 73.324 | 1.00 | 24.25 | B |
| ATOM | 4020 | CG | TYR B | 668 | 50.638 | 19.089 | 72.543 | 1.00 | 24.38 | B |
| ATOM | 4021 | CD1 | TYR B | 668 | 51.752 | 19.452 | 71.785 | 1.00 | 23.71 | B |
| ATOM | 4022 | CE1 | TYR B | 668 | 51.759 | 20.610 | 71.024 | 1.00 | 24.24 | B |
| ATOM | 4023 | CD2 | TYR B | 668 | 49.519 | 19.924 | 72.528 | 1.00 | 24.84 | B |
| ATOM | 4024 | CE2 | TYR B | 668 | 49.514 | 21.091 | 71.770 | 1.00 | 25.84 | B |
| ATOM | 4025 | CZ | TYR B | 668 | 50.643 | 21.426 | 71.017 | 1.00 | 26.29 | B |
| ATOM | 4026 | OH | TYR B | 668 | 50.656 | 22.572 | 70.263 | 1.00 | 27.29 | B |
| ATOM | 4027 | C | TYR B | 668 | 50.391 | 16.634 | 75.462 | 1.00 | 23.76 | B |
| ATOM | 4028 | O | TYR B | 668 | 51.364 | 15.944 | 75.751 | 1.00 | 23.84 | B |
| ATOM | 4029 | N | HIS B | 669 | 49.144 | 16.223 | 75.612 | 1.00 | 24.21 | B |
| ATOM | 4030 | CA | HIS B | 669 | 48.864 | 14.948 | 76.225 | 1.00 | 26.26 | B |
| ATOM | 4031 | CB | HIS B | 669 | 48.329 | 15.214 | 77.623 | 1.00 | 27.79 | B |
| ATOM | 4032 | CG | HIS B | 669 | 48.682 | 14.159 | 78.607 | 1.00 | 30.20 | B |
| ATOM | 4033 | CD2 | HIS B | 669 | 48.174 | 12.921 | 78.797 | 1.00 | 32.47 | B |
| ATOM | 4034 | ND1 | HIS B | 669 | 49.695 | 14.309 | 79.526 | 1.00 | 30.16 | B |
| ATOM | 4035 | CE1 | HIS B | 669 | 49.796 | 13.205 | 80.244 | 1.00 | 31.69 | B |
| ATOM | 4036 | NE2 | HIS B | 669 | 48.884 | 12.347 | 79.822 | 1.00 | 32.65 | B |
| ATOM | 4037 | C | HIS B | 669 | 47.836 | 14.157 | 75.421 | 1.00 | 25.01 | B |
| ATOM | 4038 | O | HIS B | 669 | 46.743 | 14.662 | 75.167 | 1.00 | 25.41 | B |
| ATOM | 4039 | N | HIS B | 670 | 48.163 | 12.936 | 75.003 | 1.00 | 24.99 | B |
| ATOM | 4040 | CA | HIS B | 670 | 47.173 | 12.152 | 74.262 | 1.00 | 26.67 | B |
| ATOM | 4041 | CB | HIS B | 670 | 47.788 | 10.894 | 73.644 | 1.00 | 26.67 | B |
| ATOM | 4042 | CG | HIS B | 670 | 48.526 | 11.149 | 72.367 | 1.00 | 28.25 | B |
| ATOM | 4043 | CD2 | HIS B | 670 | 49.149 | 12.266 | 71.911 | 1.00 | 27.02 | B |
| ATOM | 4044 | ND1 | HIS B | 670 | 48.704 | 10.187 | 71.399 | 1.00 | 28.10 | B |
| ATOM | 4045 | CE1 | HIS B | 670 | 49.404 | 10.697 | 70.398 | 1.00 | 27.39 | B |
| ATOM | 4046 | NE2 | HIS B | 670 | 49.685 | 11.955 | 70.687 | 1.00 | 25.82 | B |
| ATOM | 4047 | C | HIS B | 670 | 46.033 | 11.766 | 75.198 | 1.00 | 28.34 | B |
| ATOM | 4048 | O | HIS B | 670 | 46.255 | 11.292 | 76.320 | 1.00 | 28.98 | B |
| ATOM | 4049 | N | THR B | 671 | 44.812 | 11.970 | 74.717 | 1.00 | 29.18 | B |
| ATOM | 4050 | CA | THR B | 671 | 43.597 | 11.706 | 75.479 | 1.00 | 29.09 | B |
| ATOM | 4051 | CB | THR B | 671 | 42.363 | 11.963 | 74.600 | 1.00 | 28.97 | B |
| ATOM | 4052 | OG1 | THR B | 671 | 42.582 | 11.400 | 73.299 | 1.00 | 29.48 | B |
| ATOM | 4053 | CG2 | THR B | 671 | 42.108 | 13.457 | 74.470 | 1.00 | 28.78 | B |
| ATOM | 4054 | C | THR B | 671 | 43.434 | 10.341 | 76.127 | 1.00 | 27.51 | B |
| ATOM | 4055 | O | THR B | 671 | 42.911 | 10.245 | 77.234 | 1.00 | 26.92 | B |
| ATOM | 4056 | N | ASN B | 672 | 43.857 | 9.291 | 75.436 | 1.00 | 28.14 | B |
| ATOM | 4057 | CA | ASN B | 672 | 43.715 | 7.936 | 75.961 | 1.00 | 29.14 | B |
| ATOM | 4058 | CB | ASN B | 672 | 43.528 | 6.943 | 74.808 | 1.00 | 30.07 | B |
| ATOM | 4059 | CG | ASN B | 672 | 44.774 | 6.802 | 73.954 | 1.00 | 31.70 | B |
| ATOM | 4060 | OD1 | ASN B | 672 | 45.721 | 7.592 | 74.082 | 1.00 | 32.55 | B |
| ATOM | 4061 | ND2 | ASN B | 672 | 44.779 | 5.803 | 73.067 | 1.00 | 30.68 | B |
| ATOM | 4062 | C | ASN B | 672 | 44.911 | 7.538 | 76.819 | 1.00 | 27.80 | B |
| ATOM | 4063 | O | ASN B | 672 | 45.184 | 6.354 | 77.040 | 1.00 | 25.74 | B |
| ATOM | 4064 | N | LEU B | 673 | 45.624 | 8.554 | 77.283 | 1.00 | 27.49 | B |
| ATOM | 4065 | CA | LEU B | 673 | 46.771 | 8.370 | 78.152 | 1.00 | 28.57 | B |
| ATOM | 4066 | CB | LEU B | 673 | 46.267 | 8.132 | 79.581 | 1.00 | 26.43 | B |
| ATOM | 4067 | CG | LEU B | 673 | 45.295 | 9.183 | 80.132 | 1.00 | 27.88 | B |
| ATOM | 4068 | CD1 | LEU B | 673 | 44.949 | 8.826 | 81.572 | 1.00 | 28.51 | B |
| ATOM | 4069 | CD2 | LEU B | 673 | 45.898 | 10.581 | 80.067 | 1.00 | 26.47 | B |
| ATOM | 4070 | C | LEU B | 673 | 47.711 | 7.243 | 77.727 | 1.00 | 28.78 | B |
| ATOM | 4071 | O | LEU B | 673 | 48.353 | 6.613 | 78.560 | 1.00 | 28.06 | B |
| ATOM | 4072 | N | SER B | 674 | 47.805 | 6.992 | 76.431 | 1.00 | 29.36 | B |
| ATOM | 4073 | CA | SER B | 674 | 48.676 | 5.926 | 75.960 | 1.00 | 29.70 | B |
| ATOM | 4074 | CB | SER B | 674 | 48.207 | 5.459 | 74.585 | 1.00 | 32.41 | B |
| ATOM | 4075 | OG | SER B | 674 | 46.859 | 5.039 | 74.652 | 1.00 | 35.35 | B |
| ATOM | 4076 | G | SER B | 674 | 50.140 | 6.353 | 75.892 | 1.00 | 27.54 | B |
| ATOM | 4077 | O | SER B | 674 | 50.438 | 7.522 | 75.678 | 1.00 | 27.24 | B |
| ATOM | 4078 | N | ALA B | 675 | 51.053 | 5.407 | 76.074 | 1.00 | 25.24 | B |
| ATOM | 4079 | CA | ALA B | 675 | 52.455 | 5.748 | 76.006 | 1.00 | 24.53 | B |
| ATOM | 4080 | CB | ALA B | 675 | 53.304 | 4.573 | 76.393 | 1.00 | 25.14 | B |
| ATOM | 4081 | C | ALA B | 675 | 52.793 | 6.179 | 74.589 | 1.00 | 25.51 | B |
| ATOM | 4082 | O | ALA B | 675 | 52.484 | 5.472 | 73.610 | 1.00 | 26.90 | B |
| ATOM | 4083 | N | ILE B | 676 | 53.429 | 7.340 | 74.485 | 1.00 | 23.80 | B |
| ATOM | 4084 | CA | ILE B | 676 | 53.824 | 7.880 | 73.200 | 1.00 | 23.18 | B |
| ATOM | 4085 | CB | ILE B | 676 | 54.457 | 9.276 | 73.354 | 1.00 | 22.28 | B |
| ATOM | 4086 | CG2 | ILE B | 676 | 54.960 | 9.749 | 71.999 | 1.00 | 26.37 | B |
| ATOM | 4087 | CG1 | ILE B | 676 | 53.437 | 10.265 | 73.913 | 1.00 | 20.36 | B |
| ATOM | 4088 | CD1 | ILE B | 676 | 52.158 | 10.388 | 73.112 | 1.00 | 19.30 | B |
| ATOM | 4089 | C | ILE B | 676 | 54.835 | 6.966 | 72.505 | 1.00 | 23.03 | B |
| ATOM | 4090 | O | ILE B | 676 | 55.939 | 6.770 | 73.005 | 1.00 | 22.66 | B |
| ATOM | 4091 | N | THR B | 677 | 54.454 | 6.432 | 71.344 | 1.00 | 23.91 | B |
| ATOM | 4092 | CA | THR B | 677 | 55.310 | 5.536 | 70.554 | 1.00 | 24.45 | B |
| ATOM | 4093 | CB | THR B | 677 | 54.495 | 4.625 | 69.663 | 1.00 | 21.85 | B |
| ATOM | 4094 | OG1 | THR B | 677 | 53.512 | 3.966 | 70.447 | 1.00 | 21.96 | B |
| ATOM | 4095 | CG2 | THR B | 677 | 55.406 | 3.590 | 69.020 | 1.00 | 27.86 | B |
| ATOM | 4096 | C | THR B | 677 | 56.291 | 6.286 | 69.642 | 1.00 | 26.36 | B |
| ATOM | 4097 | O | THR B | 677 | 57.469 | 5.953 | 69.580 | 1.00 | 26.98 | B |
| ATOM | 4098 | N | THR B | 678 | 55.787 | 7.252 | 68.884 | 1.00 | 27.47 | B |
| ATOM | 4099 | CA | THR B | 678 | 56.647 | 8.070 | 68.046 | 1.00 | 28.85 | B |
| ATOM | 4100 | CB | THR B | 678 | 56.939 | 7.483 | 66.615 | 1.00 | 31.08 | B |
| ATOM | 4101 | OG1 | THR B | 678 | 55.731 | 7.007 | 66.003 | 1.00 | 33.29 | B |
| ATOM | 4102 | CG2 | THR B | 678 | 57.971 | 6.378 | 66.698 | 1.00 | 35.12 | B |
| ATOM | 4103 | C | THR B | 678 | 56.070 | 9.447 | 67.831 | 1.00 | 28.29 | B |
| ATOM | 4104 | O | THR B | 678 | 54.934 | 9.742 | 68.215 | 1.00 | 29.19 | B |
| ATOM | 4105 | N | PHE B | 679 | 56.889 | 10.273 | 67.194 | 1.00 | 27.12 | B |
| ATOM | 4106 | CA | PHE B | 679 | 56.552 | 11.621 | 66.835 | 1.00 | 24.71 | B |
| ATOM | 4107 | CB | PHE B | 679 | 56.246 | 12.453 | 68.085 | 1.00 | 25.37 | B |
| ATOM | 4108 | CG | PHE B | 679 | 57.481 | 12.945 | 68.829 | 1.00 | 24.77 | B |
| ATOM | 4109 | CD1 | PHE B | 679 | 58.206 | 14.061 | 68.374 | 1.00 | 21.98 | B |
| ATOM | 4110 | CD2 | PHE B | 679 | 57.916 | 12.293 | 69.990 | 1.00 | 24.36 | B |
| ATOM | 4111 | CE1 | PHE B | 679 | 59.331 | 14.509 | 69.063 | 1.00 | 19.92 | B |
| ATOM | 4112 | CE2 | PHE B | 679 | 59.032 | 12.736 | 70.671 | 1.00 | 22.18 | B |
| ATOM | 4113 | CZ | PHE B | 679 | 59.739 | 13.845 | 70.207 | 1.00 | 21.94 | B |
| ATOM | 4114 | C | PHE B | 679 | 57.762 | 12.174 | 66.095 | 1.00 | 24.89 | B |
| ATOM | 4115 | O | PHE B | 679 | 58.894 | 11.669 | 66.212 | 1.00 | 21.91 | B |
| ATOM | 4116 | N | TYR B | 680 | 57.504 | 13.231 | 65.338 | 1.00 | 25.98 | B |
| ATOM | 4117 | CA | TYR B | 680 | 58.516 | 13.918 | 64.557 | 1.00 | 25.00 | B |
| ATOM | 4118 | CB | TYR B | 680 | 58.418 | 13.438 | 63.104 | 1.00 | 23.81 | B |
| ATOM | 4119 | CG | TYR B | 680 | 59.668 | 13.711 | 62.317 | 1.00 | 27.68 | B |
| ATOM | 4120 | CD1 | TYR B | 680 | 60.795 | 12.881 | 62.429 | 1.00 | 26.50 | B |
| ATOM | 4121 | CE1 | TYR B | 680 | 62.003 | 13.232 | 61.812 | 1.00 | 27.92 | B |
| ATOM | 4122 | CD2 | TYR B | 680 | 59.780 | 14.881 | 61.559 | 1.00 | 28.23 | B |
| ATOM | 4123 | CE2 | TYR B | 680 | 60.971 | 15.237 | 60.949 | 1.00 | 27.91 | B |
| ATOM | 4124 | CZ | TYR B | 680 | 62.074 | 14.427 | 61.080 | 1.00 | 28.40 | B |
| ATOM | 4125 | OH | TYR B | 680 | 63.253 | 14.870 | 60.531 | 1.00 | 31.86 | B |
| ATOM | 4126 | C | TYR B | 680 | 58.122 | 15.405 | 64.735 | 1.00 | 23.73 | B |
| ATOM | 4127 | O | TYR B | 680 | 56.975 | 15.684 | 65.090 | 1.00 | 22.88 | B |
| ATOM | 4128 | N | VAL B | 681 | 59.053 | 16.340 | 64.520 | 1.00 | 23.01 | B |
| ATOM | 4129 | CA | VAL B | 681 | 58.785 | 17.781 | 64.698 | 1.00 | 21.87 | B |
| ATOM | 4130 | CB | VAL B | 681 | 59.211 | 18.315 | 66.071 | 1.00 | 20.14 | B |
| ATOM | 4131 | CG1 | VAL B | 681 | 58.030 | 18.505 | 66.979 | 1.00 | 18.83 | B |
| ATOM | 4132 | CG2 | VAL B | 681 | 60.254 | 17.397 | 66.641 | 1.00 | 21.44 | B |
| ATOM | 4133 | C | VAL B | 681 | 59.556 | 18.695 | 63.781 | 1.00 | 21.58 | B |
| ATOM | 4134 | O | VAL B | 681 | 60.694 | 18.427 | 63.399 | 1.00 | 19.29 | B |
| ATOM | 4135 | N | SER 8 | 682 | 58.928 | 19.818 | 63.480 | 1.00 | 21.27 | B |
| ATOM | 4136 | CA | SER B | 682 | 59.554 | 20.864 | 62.685 | 1.00 | 20.48 | B |
| ATOM | 4137 | CB | SER B | 682 | 58.923 | 20.956 | 61.300 | 1.00 | 19.96 | B |
| ATOM | 4138 | OG | SER B | 682 | 57.535 | 21.230 | 61.396 | 1.00 | 20.38 | B |
| ATOM | 4139 | C | SER B | 682 | 59.174 | 22.065 | 63.518 | 1.00 | 20.44 | B |
| ATOM | 4140 | O | SER B | 682 | 58.332 | 21.944 | 64.407 | 1.00 | 22.67 | B |
| ATOM | 4141 | N | ASP B | 683 | 59.797 | 23.207 | 63.275 | 1.00 | 19.98 | B |
| ATOM | 4142 | CA | ASP B | 683 | 59.441 | 24.394 | 64.036 | 1.00 | 19.73 | B |
| ATOM | 4143 | CB | ASP B | 683 | 60.212 | 25.638 | 63.547 | 1.00 | 20.26 | B |
| ATOM | 4144 | CG | ASP B | 683 | 61.708 | 25.650 | 63.959 | 1.00 | 21.54 | B |
| ATOM | 4145 | OD1 | ASP B | 683 | 62.132 | 24.891 | 64.860 | 1.00 | 19.41 | B |
| ATOM | 4146 | OD2 | ASP B | 683 | 62.468 | 26.460 | 63.377 | 1.00 | 22.23 | B |
| ATOM | 4147 | C | ASP B | 683 | 57.936 | 24.665 | 63.889 | 1.00 | 18.98 | B |
| ATOM | 4148 | O | ASP B | 683 | 57.351 | 25.254 | 64.782 | 1.00 | 19.03 | B |
| ATOM | 4149 | N | ASN B | 684 | 57.311 | 24.214 | 62.800 | 1.00 | 18.67 | B |
| ATOM | 4150 | CA | ASN B | 684 | 55.888 | 24.482 | 62.579 | 1.00 | 20.77 | B |
| ATOM | 4151 | CB | ASN B | 684 | 55.644 | 24.891 | 61.136 | 1.00 | 20.60 | B |
| ATOM | 4152 | CG | ASN B | 684 | 56.303 | 26.196 | 60.780 | 1.00 | 23.83 | B |
| ATOM | 4153 | OD1 | ASN B | 684 | 56.062 | 27.233 | 61.419 | 1.00 | 24.57 | B |
| ATOM | 4154 | ND2 | ASN B | 684 | 57.134 | 26.165 | 59.741 | 1.00 | 23.71 | B |
| ATOM | 4155 | C | ASN B | 684 | 54.874 | 23.392 | 62.886 | 1.00 | 22.90 | B |
| ATOM | 4156 | O | ASN B | 684 | 53.687 | 23.690 | 63.077 | 1.00 | 22.24 | B |
| ATOM | 4157 | N | ILE B | 685 | 55.327 | 22.140 | 62.890 | 1.00 | 23.24 | B |
| ATOM | 4158 | CA | ILE B | 685 | 54.449 | 20.997 | 63.118 | 1.00 | 20.82 | B |
| ATOM | 4159 | CB | ILE B | 685 | 54.156 | 20.257 | 61.778 | 1.00 | 19.24 | B |
| ATOM | 4160 | CG2 | ILE B | 685 | 53.256 | 19.057 | 62.014 | 1.00 | 18.99 | B |
| ATOM | 4161 | CG1 | ILE B | 685 | 53.488 | 21.211 | 60.784 | 1.00 | 17.58 | B |
| ATOM | 4162 | CD1 | ILE B | 685 | 52.067 | 21.562 | 61.120 | 1.00 | 17.99 | B |
| ATOM | 4163 | C | ILE B | 685 | 55.037 | 19.993 | 64.100 | 1.00 | 21.40 | B |
| ATOM | 4164 | O | ILE B | 685 | 56.263 | 19.961 | 64.315 | 1.00 | 21.31 | B |
| ATOM | 4165 | N | LEU B | 686 | 54.137 | 19.207 | 64.697 | 1.00 | 20.50 | B |
| ATOM | 4166 | CA | LEU B | 686 | 54.461 | 18.147 | 65.648 | 1.00 | 21.98 | B |
| ATOM | 4167 | CB | LEU B | 686 | 54.273 | 18.612 | 67.108 | 1.00 | 23.37 | B |
| ATOM | 4168 | CG | LEU B | 686 | 54.138 | 17.518 | 68.189 | 1.00 | 24.30 | B |
| ATOM | 4169 | CD1 | LEU B | 686 | 55.479 | 16.866 | 68.430 | 1.00 | 24.15 | B |
| ATOM | 4170 | CD2 | LEU B | 686 | 53.595 | 18.098 | 69.475 | 1.00 | 22.84 | B |
| ATOM | 4171 | C | LEU B | 686 | 53.475 | 17.026 | 65.361 | 1.00 | 22.92 | B |
| ATOM | 4172 | O | LEU B | 686 | 52.297 | 17.167 | 65.650 | 1.00 | 25.29 | B |
| ATOM | 4173 | N | VAL B | 687 | 53.927 | 15.924 | 64.780 | 1.00 | 21.99 | B |
| ATOM | 4174 | CA | VAL B | 687 | 53.018 | 14.817 | 64.511 | 1.00 | 21.64 | B |
| ATOM | 4175 | CB | VAL B | 687 | 53.186 | 14.346 | 63.000 | 1.00 | 22.41 | B |
| ATOM | 4176 | CG1 | VAL B | 687 | 52.604 | 12.949 | 62.796 | 1.00 | 21.78 | B |
| ATOM | 4177 | CG2 | VAL B | 687 | 52.476 | 15.325 | 62.052 | 1.00 | 19.88 | B |
| ATOM | 4178 | C | VAL B | 687 | 53.345 | 13.700 | 65.542 | 1.00 | 21.43 | B |
| ATOM | 4179 | O | VAL B | 687 | 54.476 | 13.202 | 65.599 | 1.00 | 21.65 | B |
| ATOM | 4180 | N | SER B | 688 | 52.387 | 13.330 | 66.390 | 1.00 | 21.20 | B |
| ATOM | 4181 | CA | SER B | 688 | 52.654 | 12.302 | 67.412 | 1.00 | 21.07 | B |
| ATOM | 4182 | CB | SER B | 688 | 52.601 | 12.920 | 68.812 | 1.00 | 21.19 | B |
| ATOM | 4183 | OG | SER B | 688 | 51.336 | 13.509 | 69.074 | 1.00 | 19.67 | B |
| ATOM | 4184 | C | SER B | 688 | 51.706 | 11.118 | 67.373 | 1.00 | 21.57 | B |
| ATOM | 4185 | O | SER B | 688 | 50.511 | 11.265 | 67.104 | 1.00 | 21.58 | B |
| ATOM | 4186 | N | GLY B | 689 | 52.221 | 9.935 | 67.674 | 1.00 | 23.31 | B |
| ATOM | 4187 | CA | GLY B | 689 | 51.348 | 8.778 | 67.635 | 1.00 | 25.20 | B |
| ATOM | 4188 | C | GLY B | 689 | 51.498 | 7.793 | 68.779 | 1.00 | 26.22 | B |
| ATOM | 4189 | O | GLY B | 689 | 52.590 | 7.549 | 69.295 | 1.00 | 26.74 | B |
| ATOM | 4190 | N | SER B | 690 | 50.372 | 7.245 | 69.200 | 1.00 | 26.47 | B |
| ATOM | 4191 | CA | SER B | 690 | 50.350 | 6.247 | 70.253 | 1.00 | 29.04 | B |
| ATOM | 4192 | CB | SER B | 690 | 49.977 | 6.881 | 71.601 | 1.00 | 30.51 | B |
| ATOM | 4193 | OG | SER B | 690 | 48.577 | 7.150 | 71.674 | 1.00 | 31.85 | B |
| ATOM | 4194 | C | SER B | 690 | 49.261 | 5.262 | 69.818 | 1.00 | 29.64 | B |
| ATOM | 4195 | O | SER B | 690 | 48.624 | 5.462 | 68.788 | 1.00 | 31.71 | B |
| ATOM | 4196 | N | GLU B | 691 | 49.037 | 4.213 | 70.599 | 1.00 | 29.52 | B |
| ATOM | 4197 | CA | GLU B | 691 | 48.005 | 3.239 | 70.280 | 1.00 | 28.36 | B |
| ATOM | 4198 | CB | GLU B | 691 | 47.857 | 2.282 | 71.452 | 1.00 | 28.07 | B |
| ATOM | 4199 | CG | GLU B | 691 | 46.563 | 1.512 | 71.496 | 1.00 | 32.75 | B |
| ATOM | 4200 | CD | GLU B | 691 | 46.531 | 0.499 | 72.635 | 1.00 | 35.90 | B |
| ATOM | 4201 | OE1 | GLU B | 691 | 47.285 | −0.507 | 72.575 | 1.00 | 37.07 | B |
| ATOM | 4202 | OE2 | GLU B | 691 | 45.751 | 0.712 | 73.594 | 1.00 | 36.72 | B |
| ATOM | 4203 | C | GLU B | 691 | 46.689 | 3.970 | 70.013 | 1.00 | 27.98 | B |
| ATOM | 4204 | O | GLU B | 691 | 46.356 | 4.921 | 70.713 | 1.00 | 29.12 | B |
| ATOM | 4205 | N | ASN B | 692 | 45.968 | 3.554 | 68.974 | 1.00 | 27.34 | B |
| ATOM | 4206 | CA | ASN B | 692 | 44.678 | 4.159 | 68.617 | 1.00 | 27.55 | B |
| ATOM | 4207 | CB | ASN B | 692 | 43.625 | 3.832 | 69.671 | 1.00 | 30.51 | B |
| ATOM | 4208 | CG | ASN B | 692 | 43.363 | 2.347 | 69.797 | 1.00 | 33.53 | B |
| ATOM | 4209 | OD1 | ASN B | 692 | 42.997 | 1.866 | 70.874 | 1.00 | 34.93 | B |
| ATOM | 4210 | ND2 | ASN B | 692 | 43.537 | 1.607 | 68.694 | 1.00 | 36.41 | B |
| ATOM | 4211 | C | ASN B | 692 | 44.671 | 5.671 | 68.417 | 1.00 | 28.06 | B |
| ATOM | 4212 | O | ASN B | 692 | 43.611 | 6.271 | 68.227 | 1.00 | 27.85 | B |
| ATOM | 4213 | N | GLN B | 693 | 45.836 | 6.300 | 68.455 | 1.00 | 27.40 | B |
| ATOM | 4214 | CA | GLN B | 693 | 45.883 | 7.737 | 68.270 | 1.00 | 26.47 | B |
| ATOM | 4215 | CB | GLN B | 693 | 46.046 | 8.410 | 69.627 | 1.00 | 26.45 | B |
| ATOM | 4216 | CG | GLN B | 693 | 44.860 | 8.235 | 70.547 | 1.00 | 27.10 | B |
| ATOM | 4217 | CD | GLN B | 693 | 44.499 | 9.523 | 71.245 | 1.00 | 27.21 | B |
| ATOM | 4218 | OE1 | GLN B | 693 | 45.360 | 10.211 | 71.776 | 1.00 | 28.92 | B |
| ATOM | 4219 | NE2 | GLN B | 693 | 43.224 | 9.859 | 71.243 | 1.00 | 29.30 | B |
| ATOM | 4220 | C | GLN B | 693 | 46.999 | 8.200 | 67.320 | 1.00 | 26.29 | B |
| ATOM | 4221 | O | GLN B | 693 | 48.105 | 7.655 | 67.332 | 1.00 | 27.55 | B |
| ATOM | 4222 | N | PHE B | 694 | 46.700 | 9.196 | 66.487 | 1.00 | 24.60 | B |
| ATOM | 4223 | CA | PHE B | 694 | 47.675 | 9.771 | 65.551 | 1.00 | 22.51 | B |
| ATOM | 4224 | CB | PHE B | 694 | 47.612 | 9.073 | 64.184 | 1.00 | 20.48 | B |
| ATOM | 4225 | CG | PHE B | 694 | 48.701 | 9.499 | 63.210 | 1.00 | 20.42 | B |
| ATOM | 4226 | CD1 | PHE B | 694 | 49.967 | 8.922 | 63.241 | 1.00 | 20.85 | B |
| ATOM | 4227 | CD2 | PHE B | 694 | 48.441 | 10.465 | 62.237 | 1.00 | 21.71 | B |
| ATOM | 4228 | CE1 | PHE B | 694 | 50.956 | 9.301 | 62.312 | 1.00 | 21.35 | B |
| ATOM | 4229 | CE2 | PHE B | 694 | 49.421 | 10.851 | 61.304 | 1.00 | 20.94 | B |
| ATOM | 4230 | CZ | PHE B | 694 | 50.674 | 10.269 | 61.343 | 1.00 | 20.97 | B |
| ATOM | 4231 | C | PHE B | 694 | 47.234 | 11.227 | 65.445 | 1.00 | 22.04 | B |
| ATOM | 4232 | O | PHE B | 694 | 46.146 | 11.508 | 64.951 | 1.00 | 22.64 | B |
| ATOM | 4233 | N | ASN B | 695 | 48.065 | 12.142 | 65.947 | 1.00 | 22.40 | B |
| ATOM | 4234 | CA | ASN B | 695 | 47.741 | 13.570 | 65.946 | 1.00 | 22.71 | B |
| ATOM | 4235 | CB | ASN B | 695 | 47.542 | 14.102 | 67.383 | 1.00 | 24.16 | B |
| ATOM | 4236 | CG | ASN B | 695 | 46.687 | 13.194 | 68.246 | 1.00 | 27.03 | B |
| ATOM | 4237 | OD1 | ASN B | 695 | 47.145 | 12.735 | 69.297 | 1.00 | 30.17 | B |
| ATOM | 4238 | ND2 | ASN B | 695 | 45.441 | 12.930 | 67.817 | 1.00 | 28.07 | B |
| ATOM | 4239 | C | ASN B | 695 | 48.762 | 14.477 | 65.261 | 1.00 | 21.75 | B |
| ATOM | 4240 | O | ASN B | 695 | 49.960 | 14.194 | 65.232 | 1.00 | 20.80 | B |
| ATOM | 4241 | N | ILE B | 696 | 48.248 | 15.588 | 64.734 | 1.00 | 21.52 | B |
| ATOM | 4242 | CA | ILE B | 696 | 49.024 | 16.622 | 64.045 | 1.00 | 20.16 | B |
| ATOM | 4243 | CB | ILE B | 696 | 48.592 | 16.711 | 62.556 | 1.00 | 17.92 | B |
| ATOM | 4244 | CG2 | ILE B | 696 | 49.367 | 17.768 | 61.821 | 1.00 | 16.21 | B |
| ATOM | 4245 | CG1 | ILE B | 696 | 48.860 | 15.371 | 61.883 | 1.00 | 16.96 | B |
| ATOM | 4246 | CD1 | ILE B | 696 | 48.335 | 15.277 | 60.471 | 1.00 | 14.78 | B |
| ATOM | 4247 | C | ILE B | 696 | 48.732 | 17.922 | 64.803 | 1.00 | 20.71 | B |
| ATOM | 4248 | O | ILE B | 696 | 47.589 | 18.207 | 65.167 | 1.00 | 20.57 | B |
| ATOM | 4249 | N | TYR B | 697 | 49.779 | 18.690 | 65.057 | 1.00 | 21.04 | B |
| ATOM | 4250 | CA | TYR B | 697 | 49.651 | 19.916 | 65.816 | 1.00 | 22.01 | B |
| ATOM | 4251 | CB | TYR B | 697 | 50.334 | 19.746 | 67.174 | 1.00 | 22.18 | B |
| ATOM | 4252 | CG | TYR B | 697 | 49.748 | 18.654 | 68.027 | 1.00 | 22.67 | B |
| ATOM | 4253 | CD1 | TYR B | 697 | 48.702 | 18.919 | 68.917 | 1.00 | 21.46 | B |
| ATOM | 4254 | CE1 | TYR B | 697 | 48.167 | 17.910 | 69.717 | 1.00 | 20.34 | B |
| ATOM | 4255 | CD2 | TYR B | 697 | 50.244 | 17.349 | 67.952 | 1.00 | 22.41 | B |
| ATOM | 4256 | CE2 | TYR B | 697 | 49.717 | 16.331 | 68.746 | 1.00 | 22.87 | B |
| ATOM | 4257 | CZ | TYR B | 697 | 48.685 | 16.615 | 69.627 | 1.00 | 21.85 | B |
| ATOM | 4258 | OH | TYR B | 697 | 48.212 | 15.607 | 70.431 | 1.00 | 22.33 | B |
| ATOM | 4259 | C | TYR B | 697 | 50.244 | 21.121 | 65.112 | 1.00 | 23.61 | B |
| ATOM | 4260 | O | TYR B | 697 | 51.334 | 21.080 | 64.538 | 1.00 | 25.65 | B |
| ATOM | 4261 | N | ASN B | 698 | 49.491 | 22.202 | 65.162 | 1.00 | 23.81 | B |
| ATOM | 4262 | CA | ASN B | 698 | 49.913 | 23.449 | 64.594 | 1.00 | 23.42 | B |
| ATOM | 4263 | CB | ASN B | 698 | 48.680 | 24.254 | 64.224 | 1.00 | 26.51 | B |
| ATOM | 4264 | CG | ASN B | 698 | 49.020 | 25.531 | 63.512 | 1.00 | 28.61 | B |
| ATOM | 4265 | OD1 | ASN B | 698 | 49.959 | 26.238 | 63.897 | 1.00 | 28.06 | B |
| ATOM | 4266 | ND2 | ASN B | 698 | 48.255 | 25.849 | 62.472 | 1.00 | 29.88 | B |
| ATOM | 4267 | C | ASN B | 698 | 50.658 | 24.079 | 65.778 | 1.00 | 23.71 | B |
| ATOM | 4268 | O | ASN B | 698 | 50.047 | 24.586 | 66.726 | 1.00 | 22.24 | B |
| ATOM | 4269 | N | LEU B | 699 | 51.980 | 23.982 | 65.732 | 1.00 | 23.60 | B |
| ATOM | 4270 | CA | LEU B | 699 | 52.841 | 24.497 | 66.773 | 1.00 | 24.06 | B |
| ATOM | 4271 | CB | LEU B | 699 | 54.270 | 24.037 | 66.517 | 1.00 | 22.91 | B |
| ATOM | 4272 | CG | LEU B | 699 | 54.837 | 22.996 | 67.475 | 1.00 | 23.91 | B |
| ATOM | 4273 | CD1 | LEU B | 699 | 53.784 | 21.972 | 67.874 | 1.00 | 24.32 | B |
| ATOM | 4274 | CD2 | LEU B | 699 | 56.028 | 22.362 | 66.832 | 1.00 | 22.79 | B |
| ATOM | 4275 | C | LEU B | 699 | 52.801 | 26.001 | 66.862 | 1.00 | 25.53 | B |
| ATOM | 4276 | O | LEU B | 699 | 53.498 | 26.580 | 67.681 | 1.00 | 27.08 | B |
| ATOM | 4277 | N | ARG B | 700 | 52.004 | 26.631 | 66.002 | 1.00 | 26.51 | B |
| ATOM | 4278 | CA | ARG B | 700 | 51.863 | 28.087 | 65.997 | 1.00 | 25.40 | B |
| ATOM | 4279 | CB | ARG B | 700 | 51.489 | 28.600 | 64.609 | 1.00 | 27.54 | B |
| ATOM | 4280 | CG | ARG B | 700 | 52.549 | 28.502 | 63.543 | 1.00 | 28.72 | B |
| ATOM | 4281 | CD | ARG B | 700 | 53.706 | 29.422 | 63.820 | 1.00 | 31.33 | B |
| ATOM | 4282 | NE | ARG B | 700 | 54.598 | 29.480 | 62.671 | 1.00 | 34.27 | B |
| ATOM | 4283 | CZ | ARG B | 700 | 54.233 | 29.908 | 61.464 | 1.00 | 36.22 | B |
| ATOM | 4284 | NH1 | ARG B | 700 | 52.989 | 30.318 | 61.259 | 1.00 | 35.49 | B |
| ATOM | 4285 | NH2 | ARG B | 700 | 55.107 | 29.920 | 60.457 | 1.00 | 38.36 | B |
| ATOM | 4286 | C | ARG B | 700 | 50.724 | 28.431 | 66.942 | 1.00 | 24.24 | B |
| ATOM | 4287 | O | ARG B | 700 | 50.914 | 29.118 | 67.928 | 1.00 | 22.77 | B |
| ATOM | 4288 | N | SER B | 701 | 49.532 | 27.949 | 66.620 | 1.00 | 23.91 | B |
| ATOM | 4289 | CA | SER B | 701 | 48.358 | 28.202 | 67.435 | 1.00 | 23.69 | B |
| ATOM | 4290 | CB | SER B | 701 | 47.105 | 27.928 | 66.633 | 1.00 | 21.95 | B |
| ATOM | 4291 | OG | SER B | 701 | 47.096 | 26.574 | 66.220 | 1.00 | 21.41 | B |
| ATOM | 4292 | C | SER B | 701 | 48.342 | 27.281 | 68.627 | 1.00 | 25.56 | B |
| ATOM | 4293 | O | SER B | 701 | 47.546 | 27.467 | 69.541 | 1.00 | 27.95 | B |
| ATOM | 4294 | N | GLY B | 702 | 49.207 | 26.273 | 68.606 | 1.00 | 25.80 | B |
| ATOM | 4295 | CA | GLY B | 702 | 49.252 | 25.311 | 69.688 | 1.00 | 25.16 | B |
| ATOM | 4296 | C | GLY B | 702 | 48.080 | 24.351 | 69.601 | 1.00 | 25.57 | B |
| ATOM | 4297 | O | GLY B | 702 | 47.963 | 23.464 | 70.427 | 1.00 | 27.20 | B |
| ATOM | 4298 | N | LYS B | 703 | 47.221 | 24.498 | 68.597 | 1.00 | 26.86 | B |
| ATOM | 4299 | CA | LYS B | 703 | 46.049 | 23.630 | 68.485 | 1.00 | 29.85 | B |
| ATOM | 4300 | CB | LYS B | 703 | 44.846 | 24.452 | 67.982 | 1.00 | 31.50 | B |
| ATOM | 4301 | CG | LYS B | 703 | 44.521 | 25.679 | 68.857 | 1.00 | 35.32 | B |
| ATOM | 4302 | CD | LYS B | 703 | 43.066 | 26.160 | 68.704 | 1.00 | 35.78 | B |
| ATOM | 4303 | CE | LYS B | 703 | 42.529 | 26.768 | 70.007 | 1.00 | 39.24 | B |
| ATOM | 4304 | NZ | LYS B | 703 | 42.643 | 25.841 | 71.218 | 1.00 | 39.02 | B |
| ATOM | 4305 | C | LYS B | 703 | 46.199 | 22.357 | 67.631 | 1.00 | 29.87 | B |
| ATOM | 4306 | O | LYS B | 703 | 47.079 | 22.245 | 66.786 | 1.00 | 31.67 | B |
| ATOM | 4307 | N | LEU B | 704 | 45.314 | 21.403 | 67.868 | 1.00 | 29.38 | B |
| ATOM | 4308 | CA | LEU B | 704 | 45.319 | 20.152 | 67.149 | 1.00 | 29.31 | B |
| ATOM | 4309 | CB | LEU B | 704 | 44.445 | 19.169 | 67.892 | 1.00 | 28.56 | B |
| ATOM | 4310 | CG | LEU B | 704 | 44.409 | 17.731 | 67.419 | 1.00 | 29.17 | B |
| ATOM | 4311 | CD1 | LEU B | 704 | 45.768 | 17.030 | 67.620 | 1.00 | 29.83 | B |
| ATOM | 4312 | CD2 | LEU B | 704 | 43.340 | 17.041 | 68.206 | 1.00 | 28.68 | B |
| ATOM | 4313 | C | LEU B | 704 | 44.732 | 20.390 | 65.772 | 1.00 | 30.18 | B |
| ATOM | 4314 | O | LEU B | 704 | 43.610 | 20.880 | 65.669 | 1.00 | 32.27 | B |
| ATOM | 4315 | N | VAL B | 705 | 45.458 | 20.053 | 64.709 | 1.00 | 30.79 | B |
| ATOM | 4316 | CA | VAL B | 705 | 44.908 | 20.275 | 63.371 | 1.00 | 31.43 | B |
| ATOM | 4317 | CB | VAL B | 705 | 46.012 | 20.551 | 62.322 | 1.00 | 30.01 | B |
| ATOM | 4318 | CG1 | VAL B | 705 | 45.368 | 20.883 | 60.976 | 1.00 | 28.32 | B |
| ATOM | 4319 | CG2 | VAL B | 705 | 46.889 | 21.685 | 62.776 | 1.00 | 29.54 | B |
| ATOM | 4320 | C | VAL B | 705 | 44.069 | 19.087 | 62.904 | 1.00 | 32.61 | B |
| ATOM | 4321 | O | VAL B | 705 | 42.930 | 19.252 | 62.462 | 1.00 | 32.10 | B |
| ATOM | 4322 | N | HIS B | 706 | 44.641 | 17.890 | 63.003 | 1.00 | 35.60 | B |
| ATOM | 4323 | CA | HIS B | 706 | 43.949 | 16.665 | 62.613 | 1.00 | 37.51 | B |
| ATOM | 4324 | CB | HIS B | 706 | 44.515 | 16.131 | 61.302 | 1.00 | 40.51 | B |
| ATOM | 4325 | CG | HIS B | 706 | 44.191 | 16.972 | 60.110 | 1.00 | 43.09 | B |
| ATOM | 4326 | CD2 | HIS B | 706 | 44.947 | 17.845 | 59.402 | 1.00 | 44.48 | B |
| ATOM | 4327 | ND1 | HIS B | 706 | 42.946 | 16.973 | 59.521 | 1.00 | 43.58 | B |
| ATOM | 4328 | CE1 | HIS B | 706 | 42.948 | 17.814 | 58.501 | 1.00 | 44.83 | B |
| ATOM | 4329 | NE2 | HIS B | 706 | 44.149 | 18.357 | 58.407 | 1.00 | 45.93 | B |
| ATOM | 4330 | C | HIS B | 706 | 44.109 | 15.583 | 63.676 | 1.00 | 37.67 | B |
| ATOM | 4331 | O | HIS B | 706 | 45.189 | 15.396 | 64.267 | 1.00 | 38.10 | B |
| ATOM | 4332 | N | ALA B | 707 | 43.032 | 14.852 | 63.917 | 1.00 | 37.33 | B |
| ATOM | 4333 | CA | ALA B | 707 | 43.104 | 13.773 | 64.891 | 1.00 | 35.31 | B |
| ATOM | 4334 | CB | ALA B | 707 | 42.368 | 14.163 | 66.163 | 1.00 | 33.61 | B |
| ATOM | 4335 | C | ALA B | 707 | 42.540 | 12.479 | 64.322 | 1.00 | 33.93 | B |
| ATOM | 4336 | O | ALA B | 707 | 42.616 | 11.455 | 64.979 | 1.00 | 33.97 | B |
| ATOM | 4337 | N | ASN B | 708 | 42.000 | 12.508 | 63.103 | 1.00 | 32.93 | B |
| ATOM | 4338 | CA | ASN B | 708 | 41.438 | 11.278 | 62.566 | 1.00 | 32.08 | B |
| ATOM | 4339 | CB | ASN B | 708 | 39.900 | 11.381 | 62.527 | 1.00 | 30.21 | B |
| ATOM | 4340 | CG | ASN B | 708 | 39.282 | 11.431 | 63.935 | 1.00 | 29.73 | B |
| ATOM | 4341 | OD1 | ASN B | 708 | 39.641 | 10.644 | 64.808 | 1.00 | 32.17 | B |
| ATOM | 4342 | ND2 | ASN B | 708 | 38.369 | 12.355 | 64.155 | 1.00 | 27.91 | B |
| ATOM | 4343 | C | ASN B | 708 | 41.998 | 10.810 | 61.227 | 1.00 | 31.64 | B |
| ATOM | 4344 | O | ASN B | 708 | 41.468 | 9.888 | 60.605 | 1.00 | 30.96 | B |
| ATOM | 4345 | N | ILE B | 709 | 43.104 | 11.412 | 60.803 | 1.00 | 31.19 | B |
| ATOM | 4346 | CA | ILE B | 709 | 43.709 | 11.050 | 59.526 | 1.00 | 29.76 | B |
| ATOM | 4347 | CB | ILE B | 709 | 45.027 | 11.813 | 59.279 | 1.00 | 30.79 | B |
| ATOM | 4348 | CG2 | ILE B | 709 | 45.414 | 11.698 | 57.830 | 1.00 | 30.87 | B |
| ATOM | 4349 | CG1 | ILE B | 709 | 44.846 | 13.292 | 59.585 | 1.00 | 31.89 | B |
| ATOM | 4350 | CD1 | ILE B | 709 | 43.831 | 13.966 | 58.690 | 1.00 | 33.84 | B |
| ATOM | 4351 | C | ILE B | 709 | 43.992 | 9.554 | 59.404 | 1.00 | 27.56 | B |
| ATOM | 4352 | O | ILE B | 709 | 43.923 | 9.003 | 58.316 | 1.00 | 28.85 | B |
| ATOM | 4353 | N | LEU B | 710 | 44.335 | 8.892 | 60.498 | 1.00 | 24.88 | B |
| ATOM | 4354 | CA | LEU B | 710 | 44.600 | 7.464 | 60.406 | 1.00 | 23.14 | B |
| ATOM | 4355 | CB | LEU B | 710 | 46.101 | 7.207 | 60.581 | 1.00 | 19.23 | B |
| ATOM | 4356 | CG | LEU B | 710 | 47.171 | 7.770 | 59.617 | 1.00 | 17.21 | B |
| ATOM | 4357 | CD1 | LEU B | 710 | 48.565 | 7.336 | 60.119 | 1.00 | 12.65 | B |
| ATOM | 4358 | CD2 | LEU B | 710 | 46.952 | 7.271 | 58.159 | 1.00 | 11.32 | B |
| ATOM | 4359 | C | LEU B | 710 | 43.787 | 6.738 | 61.491 | 1.00 | 24.82 | B |
| ATOM | 4360 | O | LEU B | 710 | 44.278 | 5.820 | 62.165 | 1.00 | 26.12 | B |
| ATOM | 4361 | N | LYS B | 711 | 42.540 | 7.172 | 61.668 | 1.00 | 24.22 | B |
| ATOM | 4362 | CA | LYS B | 711 | 41.672 | 6.583 | 62.678 | 1.00 | 23.87 | B |
| ATOM | 4363 | CB | LYS B | 711 | 40.223 | 7.113 | 62.551 | 1.00 | 23.07 | B |
| ATOM | 4364 | CG | LYS B | 711 | 39.704 | 7.286 | 61.127 | 0.00 | 24.07 | B |
| ATOM | 4365 | CD | LYS B | 711 | 39.138 | 6.000 | 60.567 | 0.00 | 24.51 | B |
| ATOM | 4366 | CE | LYS B | 711 | 38.815 | 6.171 | 59.096 | 0.00 | 24.91 | B |
| ATOM | 4367 | NZ | LYS B | 711 | 38.073 | 5.005 | 58.557 | 0.00 | 25.25 | B |
| ATOM | 4368 | C | LYS B | 711 | 41.697 | 5.066 | 62.640 | 1.00 | 22.54 | B |
| ATOM | 4369 | O | LYS B | 711 | 41.789 | 4.435 | 63.682 | 1.00 | 21.78 | B |
| ATOM | 4370 | N | ASP B | 712 | 41.656 | 4.474 | 61.450 | 1.00 | 24.59 | B |
| ATOM | 4371 | CA | ASP B | 712 | 41.649 | 3.009 | 61.346 | 1.00 | 26.66 | B |
| ATOM | 4372 | CB | ASP B | 712 | 41.358 | 2.577 | 59.902 | 1.00 | 27.40 | B |
| ATOM | 4373 | CG | ASP B | 712 | 42.533 | 2.779 | 58.971 | 1.00 | 29.97 | B |
| ATOM | 4374 | OD1 | ASP B | 712 | 43.376 | 3.660 | 59.238 | 1.00 | 31.04 | B |
| ATOM | 4375 | OD2 | ASP B | 712 | 42.601 | 2.059 | 57.945 | 1.00 | 32.95 | B |
| ATOM | 4376 | C | ASP B | 712 | 42.908 | 2.320 | 61.846 | 1.00 | 27.18 | B |
| ATOM | 4377 | O | ASP B | 712 | 42.933 | 1.100 | 61.992 | 1.00 | 28.41 | B |
| ATOM | 4378 | N | ALA B | 713 | 43.943 | 3.099 | 62.131 | 1.00 | 26.51 | B |
| ATOM | 4379 | CA | ALA B | 713 | 45.192 | 2.526 | 62.600 | 1.00 | 27.91 | B |
| ATOM | 4380 | CB | ALA B | 713 | 46.334 | 3.505 | 62.337 | 1.00 | 25.17 | B |
| ATOM | 4381 | C | ALA B | 713 | 45.117 | 2.185 | 64.091 | 1.00 | 29.57 | B |
| ATOM | 4382 | O | ALA B | 713 | 44.650 | 3.003 | 64.889 | 1.00 | 30.09 | B |
| ATOM | 4383 | N | ASP B | 714 | 45.584 | 0.991 | 64.467 | 1.00 | 30.92 | B |
| ATOM | 4384 | CA | ASP B | 714 | 45.568 | 0.564 | 65.873 | 1.00 | 33.02 | B |
| ATOM | 4385 | CB | ASP B | 714 | 45.300 | −0.947 | 65.975 | 1.00 | 34.69 | B |
| ATOM | 4386 | CG | ASP B | 714 | 43.997 | −1.348 | 65.333 | 1.00 | 36.43 | B |
| ATOM | 4387 | OD1 | ASP B | 714 | 42.933 | −0.920 | 65.810 | 1.00 | 38.16 | B |
| ATOM | 4388 | OD2 | ASP B | 714 | 44.033 | −2.089 | 64.337 | 1.00 | 40.68 | B |
| ATOM | 4389 | C | ASP B | 714 | 46.871 | 0.908 | 66.620 | 1.00 | 33.35 | B |
| ATOM | 4390 | O | ASP B | 714 | 46.871 | 1.124 | 67.837 | 1.00 | 33.65 | B |
| ATOM | 4391 | N | GLN B | 715 | 47.979 | 0.948 | 65.888 | 1.00 | 32.50 | B |
| ATOM | 4392 | CA | GLN B | 715 | 49.272 | 1.292 | 66.470 | 1.00 | 31.33 | B |
| ATOM | 4393 | CB | GLN B | 715 | 50.053 | 0.028 | 66.851 | 1.00 | 32.90 | B |
| ATOM | 4394 | CG | GLN B | 715 | 49.555 | −0.657 | 68.107 | 1.00 | 36.42 | B |
| ATOM | 4395 | CD | GLN B | 715 | 49.806 | 0.172 | 69.355 | 1.00 | 39.19 | B |
| ATOM | 4396 | OE1 | GLN B | 715 | 49.528 | −0.273 | 70.465 | 1.00 | 39.45 | B |
| ATOM | 4397 | NE2 | GLN B | 715 | 50.342 | 1.389 | 69.175 | 1.00 | 41.12 | B |
| ATOM | 4398 | CG | LN B | 715 | 50.063 | 2.123 | 65.465 | 1.00 | 29.87 | B |
| ATOM | 4399 | O | GLN B | 715 | 49.897 | 1.967 | 64.254 | 1.00 | 28.89 | B |
| ATOM | 4400 | N | ILE B | 716 | 50.885 | 3.038 | 65.968 | 1.00 | 28.35 | B |
| ATOM | 4401 | CA | ILE B | 716 | 51.712 | 3.875 | 65.102 | 1.00 | 26.58 | B |
| ATOM | 4402 | CB | ILE B | 716 | 51.360 | 5.388 | 65.216 | 1.00 | 26.71 | B |
| ATOM | 4403 | CG2 | ILE B | 716 | 52.387 | 6.226 | 64.452 | 1.00 | 24.23 | B |
| ATOM | 4404 | CG1 | ILE B | 716 | 49.979 | 5.655 | 64.611 | 1.00 | 27.09 | B |
| ATOM | 4405 | CD1 | ILE B | 716 | 48.830 | 5.042 | 65.381 | 1.00 | 29.06 | B |
| ATOM | 4406 | C | ILE B | 716 | 53.137 | 3.644 | 65.567 | 1.00 | 26.24 | B |
| ATOM | 4407 | O | ILE B | 716 | 53.563 | 4.184 | 66.583 | 1.00 | 25.69 | B |
| ATOM | 4408 | N | TRP B | 717 | 53.870 | 2.833 | 64.818 | 1.00 | 25.09 | B |
| ATOM | 4409 | CA | TRP B | 717 | 55.219 | 2.505 | 65.198 | 1.00 | 25.49 | B |
| ATOM | 4410 | CB | TRP B | 717 | 55.658 | 1.210 | 64.497 | 1.00 | 28.30 | B |
| ATOM | 4411 | CG | TRP B | 717 | 54.780 | 0.010 | 64.877 | 1.00 | 31.04 | B |
| ATOM | 4412 | CD2 | TRP B | 717 | 54.454 | −0.438 | 66.203 | 1.00 | 29.45 | B |
| ATOM | 4413 | CE2 | TRP B | 717 | 53.559 | −1.521 | 66.071 | 1.00 | 29.85 | B |
| ATOM | 4414 | CE3 | TRP B | 717 | 54.828 | −0.029 | 67.483 | 1.00 | 30.32 | B |
| ATOM | 4415 | CD1 | TRP B | 717 | 54.091 | −0.807 | 64.024 | 1.00 | 30.88 | B |
| ATOM | 4416 | NE1 | TRP B | 717 | 53.353 | −1.724 | 64.736 | 1.00 | 29.32 | B |
| ATOM | 4417 | CZ2 | TRP B | 717 | 53.027 | −2.199 | 67.172 | 1.00 | 33.01 | B |
| ATOM | 4418 | CZ3 | TRP B | 717 | 54.295 | −0.706 | 68.583 | 1.00 | 33.00 | B |
| ATOM | 4419 | CH2 | TRP B | 717 | 53.406 | −1.778 | 68.416 | 1.00 | 32.26 | B |
| ATOM | 4420 | C | TRP B | 717 | 56.207 | 3.611 | 64.925 | 1.00 | 25.13 | B |
| ATOM | 4421 | O | TRP B | 717 | 56.995 | 3.961 | 65.782 | 1.00 | 27.18 | B |
| ATOM | 4422 | N | SER B | 718 | 56.163 | 4.189 | 63.743 | 1.00 | 24.84 | B |
| ATOM | 4423 | CA | SER B | 718 | 57.127 | 5.212 | 63.423 | 1.00 | 24.34 | B |
| ATOM | 4424 | CB | SER B | 718 | 58.314 | 4.536 | 62.727 | 1.00 | 24.77 | B |
| ATOM | 4425 | OG | SER B | 718 | 59.315 | 5.464 | 62.352 | 1.00 | 29.36 | B |
| ATOM | 4426 | C | SER B | 718 | 56.477 | 6.265 | 62.533 | 1.00 | 23.95 | B |
| ATOM | 4427 | O | SER B | 718 | 55.668 | 5.949 | 61.663 | 1.00 | 24.34 | B |
| ATOM | 4428 | N | VAL B | 719 | 56.813 | 7.521 | 62.754 | 1.00 | 21.34 | B |
| ATOM | 4429 | CA | VAL B | 719 | 56.236 | 8.549 | 61.934 | 1.00 | 20.74 | B |
| ATOM | 4430 | CB | VAL B | 719 | 55.041 | 9.202 | 62.663 | 1.00 | 20.34 | B |
| ATOM | 4431 | CG1 | VAL B | 719 | 55.531 | 10.069 | 63.799 | 1.00 | 20.79 | B |
| ATOM | 4432 | CG2 | VAL B | 719 | 54.182 | 9.964 | 61.685 | 1.00 | 20.98 | B |
| ATOM | 4433 | C | VAL B | 719 | 57.355 | 9.538 | 61.608 | 1.00 | 21.48 | B |
| ATOM | 4434 | O | VAL B | 719 | 58.204 | 9.840 | 62.452 | 1.00 | 21.37 | B |
| ATOM | 4435 | N | ASN B | 720 | 57.391 | 10.001 | 60.368 | 1.00 | 20.75 | B |
| ATOM | 4436 | CA | ASN B | 720 | 58.434 | 10.915 | 59.950 | 1.00 | 20.33 | B |
| ATOM | 4437 | CB | ASN B | 720 | 59.582 | 10.130 | 59.331 | 1.00 | 19.95 | B |
| ATOM | 4438 | CG | ASN B | 720 | 60.744 | 11.013 | 58.939 | 1.00 | 20.71 | B |
| ATOM | 4439 | OD1 | ASN B | 720 | 60.570 | 12.186 | 58.613 | 1.00 | 22.75 | B |
| ATOM | 4440 | ND2 | ASN B | 720 | 61.934 | 10.452 | 58.960 | 1.00 | 19.81 | B |
| ATOM | 4441 | C | ASN B | 720 | 57.831 | 11.792 | 58.885 | 1.00 | 21.17 | B |
| ATOM | 4442 | O | ASN B | 720 | 57.037 | 11.318 | 58.078 | 1.00 | 23.00 | B |
| ATOM | 4443 | N | PHE B | 721 | 58.187 | 13.067 | 58.850 | 1.00 | 20.66 | B |
| ATOM | 4444 | CA | PHE B | 721 | 57.630 | 13.907 | 57.791 | 1.00 | 21.75 | B |
| ATOM | 4445 | CB | PHE B | 721 | 56.339 | 14.591 | 58.289 | 1.00 | 17.46 | B |
| ATOM | 4446 | CG | PHE B | 721 | 56.556 | 15.653 | 59.312 | 1.00 | 14.32 | B |
| ATOM | 4447 | CO1 | PHE B | 721 | 56.870 | 16.939 | 58.921 | 1.00 | 10.85 | B |
| ATOM | 4448 | CD2 | PHE B | 721 | 56.404 | 15.387 | 60.660 | 1.00 | 11.84 | B |
| ATOM | 4449 | CE1 | PHE B | 721 | 57.028 | 17.953 | 59.855 | 1.00 | 10.06 | B |
| ATOM | 4450 | CE2 | PHE B | 721 | 56.561 | 16.405 | 61.607 | 1.00 | 13.14 | B |
| ATOM | 4451 | CZ | PHE B | 721 | 56.873 | 17.693 | 61.198 | 1.00 | 12.02 | B |
| ATOM | 4452 | C | PHE B | 721 | 58.656 | 14.902 | 57.259 | 1.00 | 22.48 | B |
| ATOM | 4453 | O | PHE B | 721 | 59.528 | 15.344 | 58.000 | 1.00 | 21.67 | B |
| ATOM | 4454 | N | LYS B | 722 | 58.584 | 15.205 | 55.961 | 1.00 | 24.01 | B |
| ATOM | 4455 | CA | LYS B | 722 | 59.508 | 16.161 | 55.320 | 1.00 | 24.93 | B |
| ATOM | 4456 | CB | LYS B | 722 | 60.752 | 15.450 | 54.783 | 1.00 | 25.59 | B |
| ATOM | 4457 | CG | LYS B | 722 | 61.838 | 15.134 | 55.800 | 1.00 | 27.22 | B |
| ATOM | 4458 | CD | LYS B | 722 | 62.834 | 16.283 | 55.958 | 0.00 | 27.08 | B |
| ATOM | 4459 | CE | LYS B | 722 | 62.210 | 17.519 | 56.576 | 0.00 | 27.46 | B |
| ATOM | 4460 | NZ | LYS B | 722 | 63.225 | 18.601 | 56.697 | 0.00 | 27.62 | B |
| ATOM | 4461 | C | LYS B | 722 | 58.838 | 16.863 | 54.151 | 1.00 | 24.47 | B |
| ATOM | 4462 | O | LYS B | 722 | 58.571 | 16.242 | 53.121 | 1.00 | 25.21 | B |
| ATOM | 4463 | N | GLY B | 723 | 58.569 | 18.153 | 54.298 | 1.00 | 24.23 | B |
| ATOM | 4464 | CA | GLY B | 723 | 57.937 | 18.872 | 53.209 | 1.00 | 26.31 | B |
| ATOM | 4465 | C | GLY B | 723 | 56.464 | 18.536 | 53.019 | 1.00 | 28.74 | B |
| ATOM | 4466 | O | GLY B | 723 | 55.694 | 18.529 | 53.979 | 1.00 | 29.75 | B |
| ATOM | 4467 | N | LYS B | 724 | 56.057 | 18.235 | 51.794 | 1.00 | 28.84 | B |
| ATOM | 4468 | CA | LYS B | 724 | 54.652 | 17.941 | 51.530 | 1.00 | 29.80 | B |
| ATOM | 4469 | CB | LYS B | 724 | 54.352 | 18.219 | 50.074 | 1.00 | 31.52 | B |
| ATOM | 4470 | CG | LYS B | 724 | 55.436 | 17.698 | 49.171 | 1.00 | 32.34 | B |
| ATOM | 4471 | CD | LYS B | 724 | 54.881 | 16.783 | 48.115 | 1.00 | 33.78 | B |
| ATOM | 4472 | CE | LYS B | 724 | 55.974 | 16.447 | 47.107 | 1.00 | 35.08 | B |
| ATOM | 4473 | NZ | LYS B | 724 | 56.379 | 17.659 | 46.344 | 1.00 | 33.47 | B |
| ATOM | 4474 | C | LYS B | 724 | 54.184 | 16.535 | 51.841 | 1.00 | 29.68 | B |
| ATOM | 4475 | O | LYS B | 724 | 52.988 | 16.260 | 51.801 | 1.00 | 28.58 | B |
| ATOM | 4476 | N | THR B | 725 | 55.121 | 15.643 | 52.135 | 1.00 | 29.01 | B |
| ATOM | 4477 | CA | THR B | 725 | 54.768 | 14.261 | 52.412 | 1.00 | 28.07 | B |
| ATOM | 4478 | CB | THR B | 725 | 55.459 | 13.315 | 51.372 | 1.00 | 28.24 | B |
| ATOM | 4479 | OG1 | THR B | 725 | 55.740 | 12.040 | 51.957 | 1.00 | 28.44 | B |
| ATOM | 4480 | CG2 | THR B | 725 | 56.732 | 13.943 | 50.850 | 1.00 | 29.05 | B |
| ATOM | 4481 | C | THR B | 725 | 55.081 | 13.853 | 53.847 | 1.00 | 27.60 | B |
| ATOM | 4482 | O | THR B | 725 | 56.070 | 14.292 | 54.445 | 1.00 | 27.85 | B |
| ATOM | 4483 | N | LEU B | 726 | 54.190 | 13.036 | 54.399 | 1.00 | 26.00 | B |
| ATOM | 4484 | CA | LEU B | 726 | 54.310 | 12.528 | 55.755 | 1.00 | 24.86 | B |
| ATOM | 4485 | CB | LEU B | 726 | 53.147 | 13.030 | 56.612 | 1.00 | 23.41 | B |
| ATOM | 4486 | CG | LEU B | 726 | 52.907 | 12.266 | 57.924 | 1.00 | 21.65 | B |
| ATOM | 4487 | OD1 | LEU B | 726 | 54.123 | 12.369 | 58.805 | 1.00 | 24.41 | B |
| ATOM | 4488 | CD2 | LEU B | 726 | 51.698 | 12.816 | 58.643 | 1.00 | 21.21 | B |
| ATOM | 4489 | C | LEU B | 726 | 54.254 | 11.014 | 55.677 | 1.00 | 25.90 | B |
| ATOM | 4490 | O | LEU B | 726 | 53.370 | 10.464 | 55.019 | 1.00 | 27.58 | B |
| ATOM | 4491 | N | VAL B | 727 | 55.196 | 10.329 | 56.315 | 1.00 | 24.78 | B |
| ATOM | 4492 | CA | VAL B | 727 | 55.151 | 8.875 | 56.283 | 1.00 | 25.06 | B |
| ATOM | 4493 | CB | VAL B | 727 | 56.388 | 8.256 | 55.556 | 1.00 | 23.86 | B |
| ATOM | 4494 | CG1 | VAL B | 727 | 56.432 | 8.730 | 54.129 | 1.00 | 22.56 | B |
| ATOM | 4495 | CG2 | VAL B | 727 | 57.678 | 8.571 | 56.296 | 1.00 | 23.51 | B |
| ATOM | 4496 | C | VAL B | 727 | 55.009 | 8.257 | 57.680 | 1.00 | 25.61 | B |
| ATOM | 4497 | O | VAL B | 727 | 55.637 | 8.717 | 58.641 | 1.00 | 25.40 | B |
| ATOM | 4498 | N | ALA B | 728 | 54.163 | 7.224 | 57.775 | 1.00 | 26.51 | B |
| ATOM | 4499 | CA | ALA B | 728 | 53.898 | 6.501 | 59.028 | 1.00 | 27.01 | B |
| ATOM | 4500 | CB | ALA B | 728 | 52.656 | 7.087 | 59.692 | 1.00 | 25.86 | B |
| ATOM | 4501 | C | ALA B | 728 | 53.734 | 4.960 | 58.886 | 1.00 | 27.72 | B |
| ATOM | 4502 | O | ALA B | 728 | 52.981 | 4.480 | 58.039 | 1.00 | 28.25 | B |
| ATOM | 4503 | N | ALA B | 729 | 54.457 | 4.204 | 59.718 | 1.00 | 29.53 | B |
| ATOM | 4504 | CA | ALA B | 729 | 54.401 | 2.735 | 59.745 | 1.00 | 30.38 | B |
| ATOM | 4505 | CB | ALA B | 729 | 55.761 | 2.150 | 60.042 | 1.00 | 26.88 | B |
| ATOM | 4506 | C | ALA B | 729 | 53.441 | 2.404 | 60.876 | 1.00 | 32.48 | B |
| ATOM | 4507 | O | ALA B | 729 | 53.692 | 2.754 | 62.036 | 1.00 | 32.95 | B |
| ATOM | 4508 | N | VAL B | 730 | 52.358 | 1.710 | 60.541 | 1.00 | 34.76 | B |
| ATOM | 4509 | CA | VAL B | 730 | 51.326 | 1.409 | 61.520 | 1.00 | 36.48 | B |
| ATOM | 4510 | CB | VAL B | 730 | 50.133 | 2.339 | 61.277 | 1.00 | 35.80 | B |
| ATOM | 4511 | CG1 | VAL B | 730 | 50.546 | 3.802 | 61.484 | 1.00 | 35.67 | B |
| ATOM | 4512 | CG2 | VAL B | 730 | 49.642 | 2.148 | 59.859 | 1.00 | 35.12 | B |
| ATOM | 4513 | C | VAL B | 730 | 50.819 | −0.022 | 61.471 | 1.00 | 37.73 | B |
| ATOM | 4514 | O | VAL B | 730 | 51.069 | −0.738 | 60.507 | 1.00 | 39.94 | B |
| ATOM | 4515 | N | GLU B | 731 | 50.102 | −0.422 | 62.520 | 1.00 | 38.43 | B |
| ATOM | 4516 | CA | GLU B | 731 | 49.509 | −1.755 | 62.608 | 1.00 | 38.21 | B |
| ATOM | 4517 | CB | GLU B | 731 | 49.851 | −2.413 | 63.929 | 1.00 | 37.52 | B |
| ATOM | 4518 | CG | GLU B | 731 | 49.515 | −3.869 | 63.933 | 1.00 | 39.46 | B |
| ATOM | 4519 | CD | GLU B | 731 | 49.255 | −4.398 | 65.328 | 1.00 | 41.40 | B |
| ATOM | 4520 | OE1 | GLU B | 731 | 48.163 | −4.105 | 65.873 | 1.00 | 41.33 | B |
| ATOM | 4521 | OE2 | GLU B | 731 | 50.140 | −5.101 | 65.877 | 1.00 | 42.25 | B |
| ATOM | 4522 | C | GLU B | 731 | 47.996 | −1.611 | 62.504 | 1.00 | 39.22 | B |
| ATOM | 4523 | O | GLU B | 731 | 47.393 | −0.781 | 63.189 | 1.00 | 38.41 | B |
| ATOM | 4524 | N | LYS B | 732 | 47.382 | −2.424 | 61.652 | 1.00 | 41.10 | B |
| ATOM | 4525 | CA | LYS B | 732 | 45.938 | −2.358 | 61.449 | 1.00 | 43.00 | B |
| ATOM | 4526 | CB | LYS B | 732 | 45.635 | −1.485 | 60.226 | 1.00 | 42.61 | B |
| ATOM | 4527 | CG | LYS B | 732 | 46.197 | −0.077 | 60.333 | 0.00 | 43.37 | B |
| ATOM | 4528 | CD | LYS B | 732 | 46.607 | 0.492 | 58.981 | 0.00 | 43.64 | B |
| ATOM | 4529 | CE | LYS B | 732 | 45.431 | 0.678 | 58.036 | 0.00 | 43.89 | B |
| ATOM | 4530 | NZ | LYS B | 732 | 45.868 | 1.358 | 56.783 | 0.00 | 44.01 | B |
| ATOM | 4531 | C | LYS B | 732 | 45.381 | −3.753 | 61.237 | 1.00 | 44.67 | B |
| ATOM | 4532 | O | LYS B | 732 | 45.879 | −4.500 | 60.393 | 1.00 | 45.72 | B |
| ATOM | 4533 | N | ASP B | 733 | 44.350 | −4.100 | 62.004 | 1.00 | 46.86 | B |
| ATOM | 4534 | CA | ASP B | 733 | 43.717 | −5.417 | 61.898 | 1.00 | 48.20 | B |
| ATOM | 4535 | CB | ASP B | 733 | 43.098 | −5.602 | 60.502 | 1.00 | 51.83 | B |
| ATOM | 4536 | CG | ASP B | 733 | 42.192 | −6.824 | 60.411 | 1.00 | 54.62 | B |
| ATOM | 4537 | OD1 | ASP B | 733 | 42.658 | −7.894 | 59.947 | 1.00 | 55.49 | B |
| ATOM | 4538 | OD2 | ASP B | 733 | 41.009 | −6.708 | 60.812 | 1.00 | 55.81 | B |
| ATOM | 4539 | C | ASP B | 733 | 44.744 | −6.520 | 62.159 | 1.00 | 46.65 | B |
| ATOM | 4540 | O | ASP B | 733 | 44.653 | −7.621 | 61.614 | 1.00 | 46.27 | B |
| ATOM | 4541 | N | GLY B | 734 | 45.739 | −6.203 | 62.977 | 1.00 | 45.55 | B |
| ATOM | 4542 | CA | GLY B | 734 | 46.749 | −7.182 | 63.308 | 1.00 | 45.42 | B |
| ATOM | 4543 | C | GLY B | 734 | 47.792 | −7.446 | 62.245 | 1.00 | 45.85 | B |
| ATOM | 4544 | O | GLY B | 734 | 48.334 | −8.548 | 62.183 | 1.00 | 46.44 | B |
| ATOM | 4545 | N | GLN B | 735 | 48.073 | −6.454 | 61.401 | 1.00 | 46.81 | B |
| ATOM | 4546 | CA | GLN B | 735 | 49.093 | −6.599 | 60.355 | 1.00 | 47.05 | B |
| ATOM | 4547 | CB | GLN B | 735 | 48.461 | −7.018 | 59.028 | 1.00 | 48.17 | B |
| ATOM | 4548 | CG | GLN B | 735 | 48.028 | −8.461 | 59.027 | 1.00 | 52.88 | B |
| ATOM | 4549 | CD | GLN B | 735 | 46.961 | −8.755 | 57.990 | 1.00 | 56.44 | B |
| ATOM | 4550 | OE1 | GLN B | 735 | 46.245 | −9.766 | 58.087 | 1.00 | 58.95 | B |
| ATOM | 4551 | NE2 | GLN B | 735 | 46.840 | −7.878 | 56.993 | 1.00 | 55.74 | B |
| ATOM | 4552 | C | GLN B | 735 | 49.897 | −5.317 | 60.176 | 1.00 | 45.22 | B |
| ATOM | 4553 | O | GLN B | 735 | 49.597 | −4.297 | 60.789 | 1.00 | 46.16 | B |
| ATOM | 4554 | N | SER B | 736 | 50.924 | −5.394 | 59.336 | 1.00 | 42.48 | B |
| ATOM | 4555 | CA | SER B | 736 | 51.817 | −4.278 | 59.061 | 1.00 | 39.38 | B |
| ATOM | 4556 | CB | SER B | 736 | 53.256 | −4.791 | 59.003 | 1.00 | 40.16 | B |
| ATOM | 4557 | OG | SER B | 736 | 54.176 | −3.797 | 59.394 | 1.00 | 40.72 | B |
| ATOM | 4558 | C | SER B | 736 | 51.468 | −3.563 | 57.753 | 1.00 | 37.61 | B |
| ATOM | 4559 | O | SER B | 736 | 51.155 | −4.193 | 56.734 | 1.00 | 37.66 | B |
| ATOM | 4560 | N | PHE B | 737 | 51.549 | −2.239 | 57.797 | 1.00 | 33.84 | B |
| ATOM | 4561 | CA | PHE B | 737 | 51.228 | −1.405 | 56.655 | 1.00 | 31.74 | B |
| ATOM | 4562 | CB | PHE B | 737 | 49.753 | −1.006 | 56.716 | 1.00 | 32.02 | B |
| ATOM | 4563 | CG | PHE B | 737 | 48.802 | −2.099 | 56.337 | 1.00 | 33.36 | B |
| ATOM | 4564 | CD1 | PHE B | 737 | 48.776 | −2.582 | 55.041 | 1.00 | 34.70 | B |
| ATOM | 4565 | CD2 | PHE B | 737 | 47.894 | −2.606 | 57.255 | 1.00 | 35.15 | B |
| ATOM | 4566 | CE1 | PHE B | 737 | 47.857 | −3.545 | 54.655 | 1.00 | 36.59 | B |
| ATOM | 4567 | CE2 | PHE B | 737 | 46.964 | −3.573 | 56.878 | 1.00 | 35.49 | B |
| ATOM | 4568 | CZ | PHE B | 737 | 46.946 | −4.044 | 55.573 | 1.00 | 36.38 | B |
| ATOM | 4569 | C | PHE B | 737 | 52.077 | −0.134 | 56.644 | 1.00 | 30.68 | B |
| ATOM | 4570 | O | PHE B | 737 | 52.650 | 0.254 | 57.665 | 1.00 | 31.62 | B |
| ATOM | 4571 | N | LEU B | 738 | 52.165 | 0.510 | 55.485 | 1.00 | 27.59 | B |
| ATOM | 4572 | CA | LEU B | 738 | 52.905 | 1.758 | 55.373 | 1.00 | 25.56 | B |
| ATOM | 4573 | CB | LEU B | 738 | 54.042 | 1.679 | 54.355 | 1.00 | 25.25 | B |
| ATOM | 4574 | CG | LEU B | 738 | 54.884 | 2.973 | 54.305 | 1.00 | 25.05 | B |
| ATOM | 4575 | CD1 | LEU B | 738 | 55.668 | 3.104 | 55.614 | 1.00 | 22.91 | B |
| ATOM | 4576 | CD2 | LEU B | 738 | 55.839 | 2.973 | 53.131 | 1.00 | 21.80 | B |
| ATOM | 4577 | C | LEU B | 738 | 51.947 | 2.841 | 54.922 | 1.00 | 24.87 | B |
| ATOM | 4578 | O | LEU B | 738 | 51.336 | 2.743 | 53.864 | 1.00 | 23.89 | B |
| ATOM | 4579 | N | GLU B | 739 | 51.820 | 3.882 | 55.728 | 1.00 | 24.71 | B |
| ATOM | 4580 | CA | GLU B | 739 | 50.940 | 4.976 | 55.372 | 1.00 | 23.29 | B |
| ATOM | 4581 | CB | GLU B | 739 | 50.206 | 5.458 | 56.612 | 1.00 | 24.09 | B |
| ATOM | 4582 | CG | GLU B | 739 | 48.692 | 5.452 | 56.495 | 1.00 | 28.17 | B |
| ATOM | 4583 | CD | GLU B | 739 | 48.058 | 4.066 | 56.517 | 1.00 | 28.62 | B |
| ATOM | 4584 | OE1 | GLU B | 739 | 48.352 | 3.273 | 57.426 | 1.00 | 31.20 | B |
| ATOM | 4585 | OE2 | GLU B | 739 | 47.232 | 3.782 | 55.633 | 1.00 | 28.53 | B |
| ATOM | 4586 | C | GLU B | 739 | 51.795 | 6.094 | 54.780 | 1.00 | 20.71 | B |
| ATOM | 4587 | O | GLU B | 739 | 52.906 | 6.329 | 55.235 | 1.00 | 20.52 | B |
| ATOM | 4588 | N | ILE B | 740 | 51.290 | 6.740 | 53.732 | 1.00 | 19.91 | B |
| ATOM | 4589 | CA | ILE B | 740 | 51.968 | 7.868 | 53.075 | 1.00 | 19.31 | B |
| ATOM | 4590 | CB | ILE B | 740 | 52.572 | 7.473 | 51.720 | 1.00 | 17.66 | B |
| ATOM | 4591 | CG2 | ILE B | 740 | 53.171 | 8.701 | 51.030 | 1.00 | 15.65 | B |
| ATOM | 4592 | CG1 | ILE B | 740 | 53.604 | 6.379 | 51.926 | 1.00 | 16.52 | B |
| ATOM | 4593 | CD1 | ILE B | 740 | 54.155 | 5.833 | 50.629 | 1.00 | 18.73 | B |
| ATOM | 4594 | C | ILE B | 740 | 50.891 | 8.923 | 52.849 | 1.00 | 19.07 | B |
| ATOM | 4595 | O | ILE B | 740 | 49.886 | 8.655 | 52.209 | 1.00 | 19.78 | B |
| ATOM | 4596 | N | LEU B | 741 | 51.106 | 10.111 | 53.395 | 1.00 | 19.72 | B |
| ATOM | 4597 | CA | LEU B | 741 | 50.151 | 11.188 | 53.259 | 1.00 | 21.30 | B |
| ATOM | 4598 | CB | LEU B | 741 | 49.728 | 11.648 | 54.645 | 1.00 | 22.07 | B |
| ATOM | 4599 | CG | LEU B | 741 | 49.077 | 10.572 | 55.530 | 1.00 | 23.25 | B |
| ATOM | 4600 | CD1 | LEU B | 741 | 48.660 | 11.188 | 56.855 | 1.00 | 23.41 | B |
| ATOM | 4601 | CD2 | LEU B | 741 | 47.856 | 9.989 | 54.825 | 1.00 | 23.48 | B |
| ATOM | 4602 | C | LEU B | 741 | 50.732 | 12.357 | 52.461 | 1.00 | 22.75 | B |
| ATOM | 4603 | O | LEU B | 741 | 51.891 | 12.745 | 52.656 | 1.00 | 24.38 | B |
| ATOM | 4604 | N | ASP B | 742 | 49.918 | 12.918 | 51.571 | 1.00 | 23.48 | B |
| ATOM | 4605 | CA | ASP B | 742 | 50.324 | 14.032 | 50.711 | 1.00 | 24.55 | B |
| ATOM | 4606 | CB | ASP B | 742 | 49.931 | 13.723 | 49.276 | 1.00 | 26.67 | B |
| ATOM | 4607 | CG | ASP B | 742 | 50.733 | 14.515 | 48.271 | 1.00 | 28.63 | B |
| ATOM | 4608 | OD1 | ASP B | 742 | 50.840 | 15.760 | 48.424 | 1.00 | 28.82 | B |
| ATOM | 4609 | OD2 | ASP B | 742 | 51.245 | 13.879 | 47.328 | 1.00 | 28.63 | B |
| ATOM | 4610 | C | ASP B | 742 | 49.688 | 15.363 | 51.103 | 1.00 | 25.06 | B |
| ATOM | 4611 | O | ASP B | 742 | 48.481 | 15.436 | 51.305 | 1.00 | 26.84 | B |
| ATOM | 4612 | N | PHE B | 743 | 50.484 | 16.423 | 51.183 | 1.00 | 25.71 | B |
| ATOM | 4613 | CA | PHE B | 743 | 49.943 | 17.731 | 51.558 | 1.00 | 27.52 | B |
| ATOM | 4614 | CB | PHE B | 743 | 50.486 | 18.191 | 52.912 | 1.00 | 26.16 | B |
| ATOM | 4615 | CG | PHE B | 743 | 49.997 | 17.388 | 54.073 | 1.00 | 24.94 | B |
| ATOM | 4616 | CD1 | PHE B | 743 | 50.565 | 16.158 | 54.378 | 1.00 | 24.17 | B |
| ATOM | 4617 | CD2 | PHE B | 743 | 48.980 | 17.887 | 54.890 | 1.00 | 23.91 | B |
| ATOM | 4618 | CE1 | PHE B | 743 | 50.132 | 15.437 | 55.490 | 1.00 | 24.90 | B |
| ATOM | 4619 | CE2 | PHE B | 743 | 48.537 | 17.184 | 56.000 | 1.00 | 21.83 | B |
| ATOM | 4620 | CZ | PHE B | 743 | 49.111 | 15.957 | 56.306 | 1.00 | 23.09 | B |
| ATOM | 4621 | C | PHE B | 743 | 50.249 | 18.808 | 50.530 | 1.00 | 29.26 | B |
| ATOM | 4622 | O | PHE B | 743 | 50.140 | 20.005 | 50.808 | 1.00 | 29.97 | B |
| ATOM | 4623 | N | SER B | 744 | 50.646 | 18.372 | 49.344 | 1.00 | 30.92 | B |
| ATOM | 4624 | CA | SER B | 744 | 50.955 | 19.277 | 48.245 | 1.00 | 32.47 | B |
| ATOM | 4625 | CB | SER B | 744 | 51.761 | 18.531 | 47.188 | 1.00 | 32.25 | B |
| ATOM | 4626 | OG | SER B | 744 | 51.080 | 17.346 | 46.824 | 1.00 | 29.83 | B |
| ATOM | 4627 | C | SER B | 744 | 49.649 | 19.790 | 47.629 | 1.00 | 34.01 | B |
| ATOM | 4628 | O | SER B | 744 | 49.524 | 21.028 | 47.432 | 1.00 | 35.67 | B |
| ATOM | 4629 | OXT | SER B | 744 | 48.778 | 18.934 | 47.348 | 1.00 | 33.32 | B |
| ATOM | 4630 | CB | SER C | 2 | 79.559 | 36.638 | 46.340 | 1.00 | 43.57 | C |
| ATOM | 4631 | OG | SER C | 2 | 79.993 | 37.690 | 45.505 | 1.00 | 44.36 | C |
| ATOM | 4632 | C | SER C | 2 | 78.062 | 35.376 | 44.763 | 1.00 | 44.40 | C |
| ATOM | 4633 | O | SER C | 2 | 78.864 | 34.452 | 44.588 | 1.00 | 45.41 | C |
| ATOM | 4634 | N | SER C | 2 | 77.555 | 35.428 | 47.144 | 1.00 | 45.64 | C |
| ATOM | 4635 | CA | SER C | 2 | 78.115 | 36.241 | 46.021 | 1.00 | 44.62 | C |
| ATOM | 4636 | N | ASN C | 3 | 77.108 | 35.678 | 43.890 | 1.00 | 43.20 | C |
| ATOM | 4637 | CA | ASN C | 3 | 76.921 | 34.908 | 42.666 | 1.00 | 41.97 | C |
| ATOM | 4638 | CB | ASN C | 3 | 75.435 | 34.551 | 42.505 | 1.00 | 43.04 | C |
| ATOM | 4639 | CG | ASN C | 3 | 74.952 | 33.552 | 43.535 | 1.00 | 43.55 | C |
| ATOM | 4640 | OD1 | ASN C | 3 | 75.330 | 32.375 | 43.512 | 1.00 | 43.93 | C |
| ATOM | 4641 | ND2 | ASN C | 3 | 74.105 | 34.017 | 44.448 | 1.00 | 43.67 | C |
| ATOM | 4642 | C | ASN C | 3 | 77.392 | 35.617 | 41.399 | 1.00 | 40.54 | C |
| ATOM | 4643 | O | ASN C | 3 | 77.723 | 36.800 | 41.401 | 1.00 | 40.39 | C |
| ATOM | 4644 | N | VAL C | 4 | 77.408 | 34.868 | 40.306 | 1.00 | 39.54 | C |
| ATOM | 4645 | CA | VAL C | 4 | 77.794 | 35.409 | 39.014 | 1.00 | 37.85 | C |
| ATOM | 4646 | CB | VAL C | 4 | 79.259 | 35.135 | 38.677 | 1.00 | 37.25 | C |
| ATOM | 4647 | CG1 | VAL C | 4 | 80.128 | 36.252 | 39.212 | 1.00 | 37.46 | C |
| ATOM | 4648 | CG2 | VAL C | 4 | 79.678 | 33.798 | 39.261 | 1.00 | 36.73 | C |
| ATOM | 4649 | C | VAL C | 4 | 76.959 | 34.717 | 37.975 | 1.00 | 36.19 | C |
| ATOM | 4650 | O | VAL C | 4 | 76.617 | 33.547 | 38.128 | 1.00 | 37.44 | C |
| ATOM | 4651 | N | VAL C | 5 | 76.627 | 35.440 | 36.917 | 1.00 | 33.92 | C |
| ATOM | 4652 | CA | VAL C | 5 | 75.844 | 34.864 | 35.846 | 1.00 | 31.07 | C |
| ATOM | 4653 | CB | VAL C | 5 | 74.812 | 35.871 | 35.315 | 1.00 | 29.79 | C |
| ATOM | 4654 | CG1 | VAL C | 5 | 74.186 | 35.342 | 34.041 | 1.00 | 30.11 | C |
| ATOM | 4655 | CG2 | VAL C | 5 | 73.735 | 36.100 | 36.359 | 1.00 | 28.61 | C |
| ATOM | 4656 | C | VAL C | 5 | 76.731 | 34.410 | 34.693 | 1.00 | 29.84 | C |
| ATOM | 4657 | O | VAL C | 5 | 77.601 | 35.147 | 34.233 | 1.00 | 29.90 | C |
| ATOM | 4658 | N | LEU C | 6 | 76.513 | 33.177 | 34.251 | 1.00 | 29.14 | C |
| ATOM | 4659 | CA | LEU C | 6 | 77.236 | 32.597 | 33.116 | 1.00 | 28.08 | C |
| ATOM | 4660 | CB | LEU C | 6 | 77.863 | 31.253 | 33.510 | 1.00 | 27.11 | C |
| ATOM | 4661 | CG | LEU C | 6 | 78.872 | 31.221 | 34.675 | 1.00 | 24.97 | C |
| ATOM | 4662 | CD1 | LEU C | 6 | 79.528 | 29.845 | 34.700 | 1.00 | 24.15 | C |
| ATOM | 4663 | CD2 | LEU C | 6 | 79.919 | 32.293 | 34.529 | 1.00 | 22.63 | C |
| ATOM | 4664 | C | LEU C | 6 | 76.201 | 32.405 | 31.990 | 1.00 | 27.51 | C |
| ATOM | 4665 | O | LEU C | 6 | 75.218 | 31.673 | 32.139 | 1.00 | 26.06 | C |
| ATOM | 4666 | N | VAL C | 7 | 76.422 | 33.083 | 30.869 | 1.00 | 28.04 | C |
| ATOM | 4667 | CA | VAL C | 7 | 75.498 | 33.035 | 29.732 | 1.00 | 29.08 | C |
| ATOM | 4668 | CB | VAL C | 7 | 75.410 | 34.425 | 29.046 | 1.00 | 30.10 | C |
| ATOM | 4669 | CG1 | VAL C | 7 | 74.517 | 34.349 | 27.805 | 1.00 | 30.31 | C |
| ATOM | 4670 | CG2 | VAL C | 7 | 74.887 | 35.454 | 30.033 | 1.00 | 28.75 | C |
| ATOM | 4671 | C | VAL C | 7 | 75.861 | 31.988 | 28.678 | 1.00 | 29.01 | C |
| ATOM | 4672 | O | VAL C | 7 | 77.008 | 31.913 | 28.226 | 1.00 | 28.23 | C |
| ATOM | 4673 | N | SER C | 8 | 74.865 | 31.191 | 28.291 | 1.00 | 28.51 | C |
| ATOM | 4674 | CA | SER C | 8 | 75.063 | 30.147 | 27.296 | 1.00 | 27.88 | C |
| ATOM | 4675 | CB | SER C | 8 | 74.009 | 29.037 | 27.418 | 1.00 | 28.20 | C |
| ATOM | 4676 | OG | SER C | 8 | 72.804 | 29.396 | 26.748 | 1.00 | 25.94 | C |
| ATOM | 4677 | C | SER C | 8 | 74.965 | 30.741 | 25.898 | 1.00 | 28.40 | C |
| ATOM | 4678 | O | SER C | 8 | 74.359 | 31.803 | 25.707 | 1.00 | 27.07 | C |
| ATOM | 4679 | N | GLY C | 9 | 75.574 | 30.034 | 24.941 | 1.00 | 27.46 | C |
| ATOM | 4680 | CA | GLY C | 9 | 75.569 | 30.450 | 23.553 | 1.00 | 26.64 | C |
| ATOM | 4681 | C | GLY C | 9 | 74.142 | 30.602 | 23.067 | 1.00 | 26.05 | C |
| ATOM | 4682 | O | GLY C | 9 | 73.874 | 31.296 | 22.085 | 1.00 | 26.15 | C |
| ATOM | 4683 | N | GLU C | 10 | 73.219 | 29.941 | 23.752 | 1.00 | 24.38 | C |
| ATOM | 4684 | CA | GLU C | 10 | 71.824 | 30.034 | 23.400 | 1.00 | 23.90 | C |
| ATOM | 4685 | CB | GLU C | 10 | 71.116 | 28.716 | 23.732 | 1.00 | 24.63 | C |
| ATOM | 4686 | CG | GLU C | 10 | 71.338 | 27.635 | 22.678 | 1.00 | 29.19 | C |
| ATOM | 4687 | CD | GLU C | 10 | 71.102 | 26.213 | 23.204 | 1.00 | 33.43 | C |
| ATOM | 4688 | OE1 | GLU C | 10 | 70.758 | 25.307 | 22.400 | 1.00 | 35.21 | C |
| ATOM | 4689 | OE2 | GLU C | 10 | 71.281 | 25.992 | 24.420 | 1.00 | 36.33 | C |
| ATOM | 4690 | C | GLU C | 10 | 71.186 | 31.221 | 24.133 | 1.00 | 23.12 | C |
| ATOM | 4691 | O | GLU C | 10 | 69.975 | 31.374 | 24.128 | 1.00 | 20.55 | C |
| ATOM | 4692 | N | GLY C | 11 | 72.008 | 32.064 | 24.756 | 1.00 | 25.05 | C |
| ATOM | 4693 | CA | GLY C | 11 | 71.491 | 33.228 | 25.458 | 1.00 | 26.75 | C |
| ATOM | 4694 | C | GLY C | 11 | 70.939 | 33.036 | 26.861 | 1.00 | 29.02 | C |
| ATOM | 4695 | O | GLY C | 11 | 70.676 | 34.022 | 27.557 | 1.00 | 28.98 | C |
| ATOM | 4696 | N | GLU C | 12 | 70.757 | 31.782 | 27.276 | 1.00 | 29.73 | C |
| ATOM | 4697 | CA | GLU C | 12 | 70.238 | 31.469 | 28.604 | 1.00 | 29.91 | C |
| ATOM | 4698 | CB | GLU C | 12 | 69.778 | 30.009 | 28.657 | 1.00 | 29.67 | C |
| ATOM | 4699 | CG | GLU C | 12 | 68.537 | 29.805 | 27.817 | 1.00 | 29.78 | C |
| ATOM | 4700 | CD | GLU C | 12 | 67.792 | 28.516 | 28.112 | 1.00 | 31.45 | C |
| ATOM | 4701 | OE1 | GLU C | 12 | 68.320 | 27.419 | 27.801 | 1.00 | 31.80 | C |
| ATOM | 4702 | OE2 | GLU C | 12 | 66.663 | 28.603 | 28.651 | 1.00 | 32.30 | C |
| ATOM | 4703 | C | GLU C | 12 | 71.231 | 31.769 | 29.730 | 1.00 | 31.37 | C |
| ATOM | 4704 | O | GLU C | 12 | 72.364 | 31.288 | 29.723 | 1.00 | 31.63 | C |
| ATOM | 4705 | N | ARG C | 13 | 70.793 | 32.579 | 30.693 | 1.00 | 32.45 | C |
| ATOM | 4706 | CA | ARG C | 13 | 71.642 | 32.983 | 31.807 | 1.00 | 33.31 | C |
| ATOM | 4707 | CB | ARG C | 13 | 71.215 | 34.341 | 32.335 | 1.00 | 33.47 | C |
| ATOM | 4708 | CG | ARG C | 13 | 70.328 | 35.124 | 31.392 | 1.00 | 35.00 | C |
| ATOM | 4709 | CD | ARG C | 13 | 69.909 | 36.383 | 32.092 | 1.00 | 35.56 | C |
| ATOM | 4710 | NE | ARG C | 13 | 71.082 | 37.195 | 32.375 | 1.00 | 39.21 | C |
| ATOM | 4711 | CZ | ARG C | 13 | 71.210 | 37.997 | 33.423 | 1.00 | 40.03 | C |
| ATOM | 4712 | NH1 | ARG C | 13 | 70.231 | 38.093 | 34.310 | 1.00 | 39.27 | C |
| ATOM | 4713 | NH2 | ARG C | 13 | 72.310 | 38.726 | 33.566 | 1.00 | 40.62 | C |
| ATOM | 4714 | C | ARG C | 13 | 71.607 | 31.997 | 32.949 | 1.00 | 33.28 | C |
| ATOM | 4715 | O | ARG C | 13 | 70.534 | 31.584 | 33.387 | 1.00 | 33.83 | C |
| ATOM | 4716 | N | PHE C | 14 | 72.790 | 31.626 | 33.431 | 1.00 | 33.28 | C |
| ATOM | 4717 | CA | PHE C | 14 | 72.916 | 30.692 | 34.545 | 1.00 | 32.01 | C |
| ATOM | 4718 | CB | PHE C | 14 | 73.791 | 29.488 | 34.180 | 1.00 | 32.03 | C |
| ATOM | 4719 | CG | PHE C | 14 | 73.173 | 28.552 | 33.175 | 1.00 | 32.79 | C |
| ATOM | 4720 | CD1 | PHE C | 14 | 73.005 | 28.935 | 31.850 | 1.00 | 33.70 | C |
| ATOM | 4721 | CD2 | PHE C | 14 | 72.815 | 27.256 | 33.545 | 1.00 | 32.47 | C |
| ATOM | 4722 | CE1 | PHE C | 14 | 72.491 | 28.032 | 30.910 | 1.00 | 34.61 | C |
| ATOM | 4723 | CE2 | PHE C | 14 | 72.301 | 26.349 | 32.620 | 1.00 | 30.80 | C |
| ATOM | 4724 | CZ | PHE C | 14 | 72.141 | 26.731 | 31.300 | 1.00 | 31.82 | C |
| ATOM | 4725 | C | PHE C | 14 | 73.560 | 31.394 | 35.733 | 1.00 | 31.30 | C |
| ATOM | 4726 | O | PHE C | 14 | 74.669 | 31.918 | 35.633 | 1.00 | 30.38 | C |
| ATOM | 4727 | N | THR C | 15 | 72.860 | 31.403 | 36.857 | 1.00 | 30.28 | C |
| ATOM | 4728 | CA | THR C | 15 | 73.387 | 32.013 | 38.066 | 1.00 | 29.27 | C |
| ATOM | 4729 | CB | THR C | 15 | 72.253 | 32.635 | 38.900 | 1.00 | 28.06 | C |
| ATOM | 4730 | OG1 | THR C | 15 | 71.567 | 33.612 | 38.111 | 1.00 | 27.43 | C |
| ATOM | 4731 | CG2 | THR C | 15 | 72.801 | 33.282 | 40.158 | 1.00 | 25.34 | C |
| ATOM | 4732 | C | THR C | 15 | 74.060 | 30.925 | 38.901 | 1.00 | 29.98 | C |
| ATOM | 4733 | O | THR C | 15 | 73.521 | 29.827 | 39.032 | 1.00 | 30.73 | C |
| ATOM | 4734 | N | VAL C | 16 | 75.244 | 31.211 | 39.435 | 1.00 | 30.04 | C |
| ATOM | 4735 | CA | VAL C | 16 | 75.943 | 30.254 | 40.291 | 1.00 | 31.88 | C |
| ATOM | 4736 | CB | VAL C | 16 | 76.853 | 29.283 | 39.491 | 1.00 | 32.07 | C |
| ATOM | 4737 | CG1 | VAL C | 16 | 76.035 | 28.547 | 38.455 | 1.00 | 35.42 | C |
| ATOM | 4738 | CG2 | VAL C | 16 | 77.988 | 30.034 | 38.823 | 1.00 | 34.64 | C |
| ATOM | 4739 | C | VAL C | 16 | 76.793 | 30.991 | 41.327 | 1.00 | 32.89 | C |
| ATOM | 4740 | O | VAL C | 16 | 77.097 | 32.176 | 41.168 | 1.00 | 32.60 | C |
| ATOM | 4741 | N | ASP C | 17 | 77.170 | 30.286 | 42.390 | 1.00 | 33.45 | C |
| ATOM | 4742 | CA | ASP C | 17 | 77.976 | 30.874 | 43.455 | 1.00 | 33.69 | C |
| ATOM | 4743 | CB | ASP C | 17 | 78.117 | 29.888 | 44.618 | 1.00 | 35.66 | C |
| ATOM | 4744 | CG | ASP C | 17 | 79.027 | 30.419 | 45.727 | 1.00 | 38.16 | C |
| ATOM | 4745 | OD1 | ASP C | 17 | 78.721 | 31.520 | 46.269 | 1.00 | 38.20 | C |
| ATOM | 4746 | OD2 | ASP C | 17 | 80.037 | 29.741 | 46.047 | 1.00 | 36.91 | C |
| ATOM | 4747 | C | ASP C | 17 | 79.347 | 31.221 | 42.911 | 1.00 | 33.45 | C |
| ATOM | 4748 | O | ASP C | 17 | 80.034 | 30.352 | 42.391 | 1.00 | 32.08 | C |
| ATOM | 4749 | N | LYS C | 18 | 79.768 | 32.475 | 43.045 | 1.00 | 35.48 | C |
| ATOM | 4750 | CA | LYS C | 18 | 81.068 | 32.853 | 42.495 | 1.00 | 39.15 | C |
| ATOM | 4751 | CB | LYS C | 18 | 81.378 | 34.325 | 42.766 | 1.00 | 40.50 | C |
| ATOM | 4752 | CG | LYS C | 18 | 81.886 | 34.630 | 44.144 | 1.00 | 45.70 | C |
| ATOM | 4753 | CD | LYS C | 18 | 82.169 | 36.128 | 44.285 | 1.00 | 48.13 | C |
| ATOM | 4754 | CE | LYS C | 18 | 83.245 | 36.589 | 43.318 | 1.00 | 48.38 | C |
| ATOM | 4755 | NZ | LYS C | 18 | 84.546 | 35.898 | 43.581 | 1.00 | 49.06 | C |
| ATOM | 4756 | C | LYS C | 18 | 82.212 | 31.978 | 42.993 | 1.00 | 38.99 | C |
| ATOM | 4757 | O | LYS C | 18 | 83.109 | 31.638 | 42.228 | 1.00 | 38.11 | C |
| ATOM | 4758 | N | LYS C | 19 | 82.177 | 31.596 | 44.263 | 1.00 | 39.59 | C |
| ATOM | 4759 | CA | LYS C | 19 | 83.232 | 30.759 | 44.813 | 1.00 | 38.98 | C |
| ATOM | 4760 | CB | LYS C | 19 | 82.940 | 30.429 | 46.274 | 0.00 | 39.52 | C |
| ATOM | 4761 | CG | LYS C | 19 | 84.077 | 29.703 | 46.968 | 0.00 | 40.25 | C |
| ATOM | 4762 | CD | LYS C | 19 | 83.738 | 29.363 | 48.404 | 0.00 | 40.84 | C |
| ATOM | 4763 | CE | LYS C | 19 | 82.586 | 28.380 | 48.468 | 0.00 | 41.18 | C |
| ATOM | 4764 | NZ | LYS C | 19 | 82.206 | 28.077 | 49.871 | 0.00 | 41.44 | C |
| ATOM | 4765 | C | LYS C | 19 | 83.364 | 29.470 | 44.010 | 1.00 | 38.49 | C |
| ATOM | 4766 | O | LYS C | 19 | 84.475 | 29.025 | 43.725 | 1.00 | 37.49 | C |
| ATOM | 4767 | N | ILE C | 20 | 82.223 | 28.877 | 43.651 | 1.00 | 39.86 | C |
| ATOM | 4768 | CA | ILE C | 20 | 82.167 | 27.631 | 42.858 | 1.00 | 40.16 | C |
| ATOM | 4769 | CB | ILE C | 20 | 80.725 | 27.037 | 42.836 | 1.00 | 39.38 | C |
| ATOM | 4770 | CG2 | ILE C | 20 | 80.705 | 25.698 | 42.109 | 1.00 | 38.50 | C |
| ATOM | 4771 | CG1 | ILE C | 20 | 80.225 | 26.858 | 44.270 | 1.00 | 39.70 | C |
| ATOM | 4772 | CD1 | ILE C | 20 | 78.801 | 26.351 | 44.371 | 1.00 | 41.15 | C |
| ATOM | 4773 | C | ILE C | 20 | 82.588 | 27.949 | 41.421 | 1.00 | 40.51 | C |
| ATOM | 4774 | O | ILE C | 20 | 83.424 | 27.272 | 40.841 | 1.00 | 39.84 | C |
| ATOM | 4775 | N | ALA C | 21 | 81.999 | 28.999 | 40.865 | 1.00 | 41.64 | C |
| ATOM | 4776 | CA | ALA C | 21 | 82.312 | 29.423 | 39.524 | 1.00 | 43.06 | C |
| ATOM | 4777 | CB | ALA C | 21 | 81.635 | 30.753 | 39.229 | 1.00 | 43.43 | C |
| ATOM | 4778 | C | ALA C | 21 | 83.818 | 29.554 | 39.349 | 1.00 | 43.95 | C |
| ATOM | 4779 | O | ALA C | 21 | 84.353 | 29.186 | 38.307 | 1.00 | 45.27 | C |
| ATOM | 4780 | N | GLU C | 22 | 84.506 | 30.090 | 40.354 | 1.00 | 44.60 | C |
| ATOM | 4781 | CA | GLU C | 22 | 85.958 | 30.254 | 40.266 | 1.00 | 44.95 | C |
| ATOM | 4782 | CB | GLU C | 22 | 86.546 | 30.773 | 41.582 | 1.00 | 47.05 | C |
| ATOM | 4783 | CG | GLU C | 22 | 86.200 | 32.217 | 41.915 | 1.00 | 50.65 | C |
| ATOM | 4784 | CD | GLU C | 22 | 87.063 | 32.769 | 43.035 | 1.00 | 52.85 | C |
| ATOM | 4785 | OE1 | GLU C | 22 | 87.098 | 32.164 | 44.125 | 1.00 | 54.88 | C |
| ATOM | 4786 | OE2 | GLU C | 22 | 87.716 | 33.809 | 42.826 | 1.00 | 54.85 | C |
| ATOM | 4787 | C | GLU C | 22 | 86.652 | 28.949 | 39.898 | 1.00 | 43.86 | C |
| ATOM | 4788 | O | GLU C | 22 | 87.851 | 28.933 | 39.637 | 1.00 | 43.78 | C |
| ATOM | 4789 | N | ARG C | 23 | 85.912 | 27.849 | 39.893 | 1.00 | 42.42 | C |
| ATOM | 4790 | CA | ARG C | 23 | 86.498 | 26.576 | 39.517 | 1.00 | 42.71 | C |
| ATOM | 4791 | CB | ARG C | 23 | 85.442 | 25.496 | 39.602 | 1.00 | 43.00 | C |
| ATOM | 4792 | CG | ARG C | 23 | 85.870 | 24.197 | 39.043 | 1.00 | 44.32 | C |
| ATOM | 4793 | CD | ARG C | 23 | 86.100 | 23.258 | 40.161 | 1.00 | 46.65 | C |
| ATOM | 4794 | NE | ARG C | 23 | 87.502 | 23.204 | 40.508 | 1.00 | 47.94 | C |
| ATOM | 4795 | CZ | ARG C | 23 | 87.967 | 22.532 | 41.548 | 1.00 | 48.45 | C |
| ATOM | 4796 | NE1 | ARG C | 23 | 87.122 | 21.875 | 42.338 | 1.00 | 46.40 | C |
| ATOM | 4797 | NH2 | ARG C | 23 | 89.274 | 22.493 | 41.768 | 1.00 | 50.58 | C |
| ATOM | 4798 | C | ARG C | 23 | 86.956 | 26.742 | 38.072 | 1.00 | 42.33 | C |
| ATOM | 4799 | O | ARG C | 23 | 87.703 | 25.926 | 37.536 | 1.00 | 41.75 | C |
| ATOM | 4800 | N | SER C | 24 | 86.461 | 27.812 | 37.458 | 1.00 | 42.76 | C |
| ATOM | 4801 | CA | SER C | 24 | 86.773 | 28.177 | 36.087 | 1.00 | 43.35 | C |
| ATOM | 4802 | CB | SER C | 24 | 85.531 | 28.757 | 35.406 | 1.00 | 42.29 | C |
| ATOM | 4803 | OC | SER C | 24 | 85.845 | 29.241 | 34.116 | 1.00 | 39.65 | C |
| ATOM | 4804 | C | SER C | 24 | 87.868 | 29.235 | 36.113 | 1.00 | 44.08 | C |
| ATOM | 4805 | O | SER C | 24 | 87.591 | 30.403 | 36.361 | 1.00 | 44.37 | C |
| ATOM | 4806 | N | LEU C | 25 | 89.109 | 28.830 | 35.875 | 1.00 | 45.48 | C |
| ATOM | 4807 | CA | LEU C | 25 | 90.217 | 29.773 | 35.875 | 1.00 | 47.35 | C |
| ATOM | 4808 | CB | LEU C | 25 | 91.490 | 29.117 | 35.350 | 1.00 | 48.27 | C |
| ATOM | 4809 | CG | LEU C | 25 | 92.107 | 28.027 | 36.219 | 1.00 | 48.77 | C |
| ATOM | 4810 | CD1 | LEU C | 25 | 93.392 | 27.506 | 35.595 | 0.00 | 48.78 | C |
| ATOM | 4811 | CD2 | LEU C | 25 | 92.365 | 28.610 | 37.598 | 1.00 | 48.53 | C |
| ATOM | 4812 | C | LEU C | 25 | 89.871 | 30.932 | 34.979 | 1.00 | 48.70 | C |
| ATOM | 4813 | O | LEU C | 25 | 90.293 | 32.064 | 35.233 | 1.00 | 48.05 | C |
| ATOM | 4814 | N | LEU C | 26 | 89.116 | 30.635 | 33.919 | 1.00 | 50.30 | C |
| ATOM | 4815 | CA | LEU C | 26 | 88.703 | 31.658 | 32.956 | 1.00 | 50.59 | C |
| ATOM | 4816 | CB | LEU C | 26 | 87.901 | 31.046 | 31.787 | 1.00 | 50.13 | C |
| ATOM | 4817 | CG | LEU C | 26 | 87.543 | 31.994 | 30.621 | 1.00 | 49.31 | C |
| ATOM | 4818 | CD1 | LEU C | 26 | 88.810 | 32.356 | 29.880 | 1.00 | 49.37 | C |
| ATOM | 4819 | CD2 | LEU C | 26 | 86.542 | 31.346 | 29.655 | 1.00 | 49.24 | C |
| ATOM | 4820 | C | LEU C | 26 | 87.857 | 32.687 | 33.689 | 1.00 | 50.59 | C |
| ATOM | 4821 | O | LEU C | 26 | 88.005 | 33.888 | 33.478 | 1.00 | 50.43 | C |
| ATOM | 4822 | N | LEU C | 27 | 86.980 | 32.210 | 34.562 | 1.00 | 51.20 | C |
| ATOM | 4823 | CA | LEU C | 27 | 86.132 | 33.103 | 35.325 | 1.00 | 53.37 | C |
| ATOM | 4824 | CB | LEU C | 27 | 84.930 | 32.324 | 35.871 | 1.00 | 54.39 | C |
| ATOM | 4825 | CG | LEU C | 27 | 83.811 | 33.095 | 36.583 | 1.00 | 55.55 | C |
| ATOM | 4826 | CD1 | LEU C | 27 | 84.258 | 33.408 | 37.990 | 1.00 | 56.54 | C |
| ATOM | 4827 | CD2 | LEU C | 27 | 83.445 | 34.376 | 35.817 | 1.00 | 55.58 | C |
| ATOM | 4828 | C | LEU C | 27 | 86.947 | 33.775 | 36.446 | 1.00 | 54.11 | C |
| ATOM | 4829 | O | LEU C | 27 | 86.669 | 34.918 | 36.822 | 1.00 | 54.13 | C |
| ATOM | 4830 | N | LYS C | 28 | 87.960 | 33.077 | 36.961 | 1.00 | 54.29 | C |
| ATOM | 4831 | CA | LYS C | 28 | 88.816 | 33.650 | 37.993 | 1.00 | 54.46 | C |
| ATOM | 4832 | CB | LYS C | 28 | 89.825 | 32.623 | 38.506 | 1.00 | 54.18 | C |
| ATOM | 4833 | CG | LYS C | 28 | 89.252 | 31.586 | 39.446 | 1.00 | 54.09 | C |
| ATOM | 4834 | CD | LYS C | 28 | 90.359 | 30.727 | 40.048 | 0.00 | 54.84 | C |
| ATOM | 4835 | CE | LYS C | 28 | 91.404 | 31.584 | 40.756 | 0.00 | 55.16 | C |
| ATOM | 4836 | NZ | LYS C | 28 | 90.817 | 32.412 | 41.846 | 0.00 | 55.54 | C |
| ATOM | 4837 | C | LYS C | 28 | 89.565 | 34.851 | 37.408 | 1.00 | 55.03 | C |
| ATOM | 4838 | O | LYS C | 28 | 89.502 | 35.943 | 37.956 | 1.00 | 54.89 | C |
| ATOM | 4839 | N | ASN C | 29 | 90.269 | 34.657 | 36.295 | 1.00 | 56.14 | C |
| ATOM | 4840 | CA | ASN C | 29 | 91.006 | 35.758 | 35.671 | 1.00 | 57.16 | C |
| ATOM | 4841 | CB | ASN C | 29 | 91.659 | 35.319 | 34.354 | 1.00 | 57.83 | C |
| ATOM | 4842 | CG | ASN C | 29 | 92.642 | 34.173 | 34.532 | 1.00 | 58.99 | C |
| ATOM | 4843 | OD1 | ASN C | 29 | 93.170 | 33.633 | 33.558 | 1.00 | 59.61 | C |
| ATOM | 4844 | ND2 | ASN C | 29 | 92.895 | 33.800 | 35.776 | 1.00 | 59.68 | C |
| ATOM | 4845 | C | ASN C | 29 | 90.071 | 36.923 | 35.375 | 1.00 | 57.45 | C |
| ATOM | 4846 | O | ASN C | 29 | 90.455 | 38.079 | 35.525 | 1.00 | 57.22 | C |
| ATOM | 4847 | N | TYR C | 30 | 88.849 | 36.620 | 34.943 | 1.00 | 57.89 | C |
| ATOM | 4848 | CA | TYR C | 30 | 87.882 | 37.664 | 34.615 | 1.00 | 58.66 | C |
| ATOM | 4849 | CB | TYR C | 30 | 86.516 | 37.039 | 34.317 | 1.00 | 57.87 | C |
| ATOM | 4850 | CG | TYR C | 30 | 85.636 | 37.872 | 33.409 | 1.00 | 56.71 | C |
| ATOM | 4851 | CD1 | TYR C | 30 | 86.054 | 38.229 | 32.133 | 1.00 | 55.67 | C |
| ATOM | 4852 | CE1 | TYR C | 30 | 85.231 | 38.968 | 31.282 | 1.00 | 55.50 | C |
| ATOM | 4853 | CD2 | TYR C | 30 | 84.373 | 38.276 | 33.818 | 1.00 | 56.61 | C |
| ATOM | 4854 | CE2 | TYR C | 30 | 83.539 | 39.014 | 32.979 | 1.00 | 56.59 | C |
| ATOM | 4855 | CZ | TYR C | 30 | 83.972 | 39.355 | 31.712 | 1.00 | 56.92 | C |
| ATOM | 4856 | OH | TYR C | 30 | 83.132 | 40.065 | 30.873 | 1.00 | 57.63 | C |
| ATOM | 4857 | C | TYR C | 30 | 87.787 | 38.649 | 35.783 | 1.00 | 60.59 | C |
| ATOM | 4858 | O | TYR C | 30 | 87.482 | 39.830 | 35.590 | 1.00 | 59.71 | C |
| ATOM | 4859 | N | LEU C | 31 | 88.062 | 38.146 | 36.989 | 1.00 | 62.33 | C |
| ATOM | 4860 | CA | LEU C | 31 | 88.047 | 38.932 | 38.230 | 1.00 | 62.29 | C |
| ATOM | 4861 | CB | LEU C | 31 | 89.371 | 39.712 | 38.370 | 1.00 | 62.08 | C |
| ATOM | 4862 | CG | LEU C | 31 | 89.853 | 40.603 | 37.215 | 1.00 | 61.04 | C |
| ATOM | 4863 | CO1 | LEU C | 31 | 89.303 | 42.000 | 37.370 | 1.00 | 60.84 | C |
| ATOM | 4864 | CD2 | LEU C | 31 | 91.363 | 40.639 | 37.205 | 1.00 | 61.22 | C |
| ATOM | 4865 | C | LEU C | 31 | 86.860 | 39.875 | 38.358 | 1.00 | 62.55 | C |
| ATOM | 4866 | O | LEU C | 31 | 85.754 | 39.555 | 37.917 | 1.00 | 63.88 | C |
| ATOM | 4867 | N | ILE C | 45 | 80.545 | 40.215 | 37.240 | 1.00 | 45.54 | C |
| ATOM | 4868 | CA | ILE C | 45 | 79.354 | 39.523 | 37.719 | 1.00 | 45.75 | C |
| ATOM | 4869 | CB | ILE C | 45 | 78.323 | 40.511 | 38.318 | 1.00 | 46.06 | C |
| ATOM | 4870 | CG2 | ILE C | 45 | 77.704 | 41.376 | 37.213 | 1.00 | 43.89 | C |
| ATOM | 4871 | CG1 | ILE C | 45 | 77.248 | 39.723 | 39.075 | 1.00 | 46.70 | C |
| ATOM | 4872 | CD1 | ILE C | 45 | 76.128 | 40.574 | 39.614 | 1.00 | 48.94 | C |
| ATOM | 4873 | C | ILE C | 45 | 78.665 | 38.702 | 36.617 | 1.00 | 45.24 | C |
| ATOM | 4874 | O | ILE C | 45 | 78.069 | 37.659 | 36.904 | 1.00 | 45.32 | C |
| ATOM | 4875 | N | VAL C | 46 | 78.758 | 39.158 | 35.364 | 1.00 | 43.40 | C |
| ATOM | 4876 | CA | VAL C | 46 | 78.154 | 38.438 | 34.237 | 1.00 | 41.39 | C |
| ATOM | 4877 | CB | VAL C | 46 | 76.974 | 39.226 | 33.613 | 1.00 | 41.64 | C |
| ATOM | 4878 | CG1 | VAL C | 46 | 76.308 | 38.378 | 32.531 | 1.00 | 41.02 | C |
| ATOM | 4879 | CG2 | VAL C | 46 | 75.962 | 39.604 | 34.690 | 1.00 | 41.38 | C |
| ATOM | 4880 | C | VAL C | 46 | 79.154 | 38.131 | 33.123 | 1.00 | 39.66 | C |
| ATOM | 4881 | O | VAL C | 46 | 79.480 | 39.005 | 32.318 | 1.00 | 40.66 | C |
| ATOM | 4882 | N | MSE C | 47 | 79.635 | 36.892 | 33.071 | 1.00 | 38.75 | C |
| ATOM | 4883 | CA | MSE C | 47 | 80.597 | 36.501 | 32.039 | 1.00 | 37.26 | C |
| ATOM | 4884 | CB | MSE C | 47 | 81.753 | 35.691 | 32.641 | 1.00 | 38.19 | C |
| ATOM | 4885 | CG | MSE C | 47 | 82.901 | 35.488 | 31.668 | 1.00 | 40.50 | C |
| ATOM | 4886 | SE | MSE C | 47 | 84.215 | 34.179 | 32.219 | 1.00 | 47.69 | C |
| ATOM | 4887 | CE | MSE C | 47 | 83.332 | 32.527 | 31.723 | 1.00 | 41.93 | C |
| ATOM | 4888 | C | MSE C | 47 | 79.924 | 35.669 | 30.954 | 1.00 | 34.30 | C |
| ATOM | 4889 | O | MSE C | 47 | 79.108 | 34.793 | 31.246 | 1.00 | 32.32 | C |
| ATOM | 4890 | N | PRO C | 48 | 80.240 | 35.954 | 29.682 | 1.00 | 32.53 | C |
| ATOM | 4891 | CD | PRO C | 48 | 80.789 | 37.227 | 29.204 | 1.00 | 31.72 | C |
| ATOM | 4892 | CA | PRO C | 48 | 79.655 | 35.207 | 28.566 | 1.00 | 32.32 | C |
| ATOM | 4893 | CB | PRO C | 48 | 79.787 | 36.176 | 27.389 | 1.00 | 31.12 | C |
| ATOM | 4894 | CG | PRO C | 48 | 79.872 | 37.514 | 28.049 | 1.00 | 31.73 | C |
| ATOM | 4895 | C | PRO C | 48 | 80.408 | 33.895 | 28.302 | 1.00 | 31.55 | C |
| ATOM | 4896 | O | PRO C | 48 | 81.615 | 33.809 | 28.506 | 1.00 | 32.35 | C |
| ATOM | 4897 | N | VAL C | 49 | 79.696 | 32.864 | 27.874 | 1.00 | 30.22 | C |
| ATOM | 4898 | CA | VAL C | 49 | 80.347 | 31.609 | 27.553 | 1.00 | 29.45 | C |
| ATOM | 4899 | CB | VAL C | 49 | 79.832 | 30.440 | 28.418 | 1.00 | 30.65 | C |
| ATOM | 4900 | CG1 | VAL C | 49 | 80.731 | 29.222 | 28.234 | 1.00 | 29.09 | C |
| ATOM | 4901 | CG2 | VAL C | 49 | 79.803 | 30.851 | 29.872 | 1.00 | 31.28 | C |
| ATOM | 4902 | C | VAL C | 49 | 79.958 | 31.389 | 26.105 | 1.00 | 28.38 | C |
| ATOM | 4903 | O | VAL C | 49 | 79.011 | 30.660 | 25.798 | 1.00 | 28.07 | C |
| ATOM | 4904 | N | PRO C | 50 | 80.673 | 32.049 | 25.191 | 1.00 | 27.28 | C |
| ATOM | 4905 | CD | PRO C | 50 | 81.736 | 33.036 | 25.433 | 1.00 | 27.20 | C |
| ATOM | 4906 | CA | PRO C | 50 | 80.410 | 31.937 | 23.765 | 1.00 | 26.24 | C |
| ATOM | 4907 | CB | PRO C | 50 | 81.504 | 32.810 | 23.158 | 1.00 | 26.55 | C |
| ATOM | 4908 | CG | PRO C | 50 | 81.687 | 33.866 | 24.182 | 1.00 | 25.75 | C |
| ATOM | 4909 | C | PRO C | 50 | 80.426 | 30.516 | 23.202 | 1.00 | 26.07 | C |
| ATOM | 4910 | O | PRO C | 50 | 81.292 | 29.698 | 23.529 | 1.00 | 25.12 | C |
| ATOM | 4911 | N | ASN C | 51 | 79.447 | 30.247 | 22.347 | 1.00 | 25.23 | C |
| ATOM | 4912 | CA | ASN C | 51 | 79.317 | 28.975 | 21.668 | 1.00 | 25.26 | C |
| ATOM | 4913 | CB | ASN C | 51 | 80.446 | 28.820 | 20.660 | 1.00 | 26.35 | C |
| ATOM | 4914 | CG | ASN C | 51 | 80.554 | 30.005 | 19.737 | 1.00 | 27.98 | C |
| ATOM | 4915 | OD1 | ASN C | 51 | 79.567 | 30.413 | 19.116 | 1.00 | 28.79 | C |
| ATOM | 4916 | ND2 | ASN C | 51 | 81.752 | 30.578 | 19.644 | 1.00 | 27.02 | C |
| ATOM | 4917 | C | ASN C | 51 | 79.299 | 27.762 | 22.558 | 1.00 | 24.62 | C |
| ATOM | 4918 | O | ASN C | 51 | 80.090 | 26.861 | 22.374 | 1.00 | 25.28 | C |
| ATOM | 4919 | N | VAL C | 52 | 78.388 | 27.726 | 23.510 | 1.00 | 25.26 | C |
| ATOM | 4920 | CA | VAL C | 52 | 78.284 | 26.592 | 24.395 | 1.00 | 25.46 | C |
| ATOM | 4921 | CB | VAL C | 52 | 78.983 | 26.866 | 25.714 | 1.00 | 27.00 | C |
| ATOM | 4922 | CG1 | VAL C | 52 | 78.759 | 25.698 | 26.668 | 1.00 | 26.64 | C |
| ATOM | 4923 | CG2 | VAL C | 52 | 80.473 | 27.098 | 25.487 | 1.00 | 27.68 | C |
| ATOM | 4924 | C | VAL C | 52 | 76.808 | 26.404 | 24.685 | 1.00 | 27.92 | C |
| ATOM | 4925 | O | VAL C | 52 | 76.181 | 27.275 | 25.305 | 1.00 | 30.25 | C |
| ATOM | 4926 | N | ARG C | 53 | 76.246 | 25.278 | 24.249 | 1.00 | 28.81 | C |
| ATOM | 4927 | CA | ARG C | 53 | 74.824 | 25.012 | 24.478 | 1.00 | 29.52 | C |
| ATOM | 4928 | CB | ARG C | 53 | 74.393 | 23.688 | 23.845 | 1.00 | 32.39 | C |
| ATOM | 4929 | CG | ARG C | 53 | 74.401 | 23.661 | 22.337 | 1.00 | 37.27 | C |
| ATOM | 4930 | CD | ARG C | 53 | 73.908 | 22.307 | 21.839 | 1.00 | 42.50 | C |
| ATOM | 4931 | NE | ARG C | 53 | 74.746 | 21.181 | 22.276 | 1.00 | 47.26 | C |
| ATOM | 4932 | CZ | ARG C | 53 | 74.285 | 20.079 | 22.871 | 1.00 | 49.64 | C |
| ATOM | 4933 | NH1 | ARG C | 53 | 72.984 | 19.945 | 23.114 | 1.00 | 48.76 | C |
| ATOM | 4934 | NH2 | ARG C | 53 | 75.126 | 19.106 | 23.216 | 1.00 | 50.78 | C |
| ATOM | 4935 | C | ARG C | 53 | 74.465 | 24.959 | 25.964 | 1.00 | 28.83 | C |
| ATOM | 4936 | O | ARG C | 53 | 75.226 | 24.471 | 26.794 | 1.00 | 27.65 | C |
| ATOM | 4937 | N | SER C | 54 | 73.274 | 25.454 | 26.268 | 1.00 | 27.01 | C |
| ATOM | 4938 | CA | SER C | 54 | 72.769 | 25.467 | 27.613 | 1.00 | 24.06 | C |
| ATOM | 4939 | CB | SER C | 54 | 71.302 | 25.871 | 27.612 | 1.00 | 22.38 | C |
| ATOM | 4940 | OC | SER C | 54 | 71.159 | 27.162 | 27.056 | 1.00 | 20.31 | C |
| ATOM | 4941 | C | SER C | 54 | 72.946 | 24.093 | 28.246 | 1.00 | 24.61 | C |
| ATOM | 4942 | O | SER C | 54 | 73.676 | 23.948 | 29.223 | 1.00 | 26.72 | C |
| ATOM | 4943 | N | SER C | 55 | 72.306 | 23.071 | 27.698 | 1.00 | 23.42 | C |
| ATOM | 4944 | CA | SER C | 55 | 72.455 | 21.735 | 28.268 | 1.00 | 22.33 | C |
| ATOM | 4945 | CB | SER C | 55 | 71.922 | 20.684 | 27.304 | 1.00 | 19.49 | C |
| ATOM | 4946 | OG | SER C | 55 | 72.598 | 20.765 | 26.075 | 1.00 | 22.52 | C |
| ATOM | 4947 | C | SER C | 55 | 73.909 | 21.414 | 28.610 | 1.00 | 21.51 | C |
| ATOM | 4948 | O | SER C | 55 | 74.168 | 20.766 | 29.613 | 1.00 | 23.53 | C |
| ATOM | 4949 | N | VAL C | 56 | 74.851 | 21.884 | 27.795 | 1.00 | 20.84 | C |
| ATOM | 4950 | CA | VAL C | 56 | 76.274 | 21.624 | 28.031 | 1.00 | 20.83 | C |
| ATOM | 4951 | CB | VAL C | 56 | 77.137 | 21.998 | 26.757 | 1.00 | 20.76 | C |
| ATOM | 4952 | CG1 | VAL C | 56 | 78.593 | 21.624 | 26.953 | 1.00 | 16.97 | C |
| ATOM | 4953 | CG2 | VAL C | 56 | 76.583 | 21.284 | 25.514 | 1.00 | 20.88 | C |
| ATOM | 4954 | C | VAL C | 56 | 76.768 | 22.418 | 29.260 | 1.00 | 19.61 | C |
| ATOM | 4955 | O | VAL C | 56 | 77.375 | 21.849 | 30.172 | 1.00 | 18.87 | C |
| ATOM | 4956 | N | LEU C | 57 | 76.515 | 23.724 | 29.271 | 1.00 | 19.82 | C |
| ATOM | 4957 | CA | LEU C | 57 | 76.906 | 24.588 | 30.379 | 1.00 | 20.45 | C |
| ATOM | 4958 | CB | LEU C | 57 | 76.487 | 26.026 | 30.075 | 1.00 | 17.81 | C |
| ATOM | 4959 | CG | LEU C | 57 | 76.896 | 27.099 | 31.084 | 1.00 | 15.76 | C |
| ATOM | 4960 | CD1 | LEU C | 57 | 78.375 | 27.066 | 31.281 | 1.00 | 14.13 | C |
| ATOM | 4961 | CD2 | LEU C | 57 | 76.451 | 28.463 | 30.589 | 1.00 | 17.83 | C |
| ATOM | 4962 | C | LEU C | 57 | 76.231 | 24.116 | 31.678 | 1.00 | 21.81 | C |
| ATOM | 4963 | O | LEU C | 57 | 76.842 | 24.076 | 32.744 | 1.00 | 22.15 | C |
| ATOM | 4964 | N | GLN C | 58 | 74.956 | 23.777 | 31.588 | 1.00 | 22.64 | C |
| ATOM | 4965 | CA | GLN C | 58 | 74.255 | 23.311 | 32.757 | 1.00 | 24.92 | C |
| ATOM | 4966 | CB | GLN C | 58 | 72.811 | 22.991 | 32.418 | 1.00 | 25.24 | C |
| ATOM | 4967 | CG | GLN C | 58 | 72.079 | 22.366 | 33.567 | 1.00 | 27.25 | C |
| ATOM | 4968 | CD | GLN C | 58 | 70.636 | 22.144 | 33.271 | 1.00 | 28.40 | C |
| ATOM | 4969 | OE1 | GLN C | 58 | 70.047 | 21.177 | 33.740 | 1.00 | 31.18 | C |
| ATOM | 4970 | NE2 | GLN C | 58 | 70.043 | 23.041 | 32.500 | 1.00 | 29.28 | C |
| ATOM | 4971 | C | GLN C | 58 | 74.938 | 22.065 | 33.282 | 1.00 | 26.17 | C |
| ATOM | 4972 | O | GLN C | 58 | 75.060 | 21.872 | 34.487 | 1.00 | 27.72 | C |
| ATOM | 4973 | N | LYS C | 59 | 75.375 | 21.209 | 32.373 | 1.00 | 27.32 | C |
| ATOM | 4974 | CA | LYS C | 59 | 76.047 | 19.990 | 32.771 | 1.00 | 29.09 | C |
| ATOM | 4975 | CB | LYS C | 59 | 76.229 | 19.093 | 31.549 | 1.00 | 29.32 | C |
| ATOM | 4976 | CG | LYS C | 59 | 76.548 | 17.651 | 31.869 | 1.00 | 32.80 | C |
| ATOM | 4977 | CD | LYS C | 59 | 75.309 | 16.788 | 31.972 | 1.00 | 32.66 | C |
| ATOM | 4978 | CE | LYS C | 59 | 75.708 | 15.346 | 32.280 | 1.00 | 34.41 | C |
| ATOM | 4979 | NZ | LYS C | 59 | 74.566 | 14.374 | 32.116 | 1.00 | 36.74 | C |
| ATOM | 4980 | C | LYS C | 59 | 77.406 | 20.346 | 33.412 | 1.00 | 31.23 | C |
| ATOM | 4981 | O | LYS C | 59 | 77.793 | 19.750 | 34.418 | 1.00 | 32.05 | C |
| ATOM | 4982 | N | VAL C | 60 | 78.116 | 21.320 | 32.837 | 1.00 | 31.14 | C |
| ATOM | 4983 | CA | VAL C | 60 | 79.416 | 21.769 | 33.350 | 1.00 | 30.44 | C |
| ATOM | 4984 | CB | VAL C | 60 | 80.022 | 22.893 | 32.461 | 1.00 | 30.16 | C |
| ATOM | 4985 | CG1 | VAL C | 60 | 81.236 | 23.514 | 33.139 | 1.00 | 28.33 | C |
| ATOM | 4986 | CG2 | VAL C | 60 | 80.422 | 22.325 | 31.109 | 1.00 | 32.07 | C |
| ATOM | 4987 | C | VAL C | 60 | 79.310 | 22.314 | 34.767 | 1.00 | 31.68 | C |
| ATOM | 4988 | O | VAL C | 60 | 80.078 | 21.953 | 35.650 | 1.00 | 32.71 | C |
| ATOM | 4989 | N | ILE C | 61 | 78.356 | 23.204 | 34.969 | 1.00 | 32.12 | C |
| ATOM | 4990 | CA | ILE C | 61 | 78.140 | 23.804 | 36.262 | 1.00 | 31.24 | C |
| ATOM | 4991 | CB | ILE C | 61 | 77.021 | 24.824 | 36.170 | 1.00 | 31.54 | G |
| ATOM | 4992 | CG2 | ILE C | 61 | 76.601 | 25.279 | 37.564 | 1.00 | 28.63 | G |
| ATOM | 4993 | CG1 | ILE C | 61 | 77.497 | 25.982 | 35.297 | 1.00 | 31.49 | G |
| ATOM | 4994 | CD1 | ILE C | 61 | 76.388 | 26.850 | 34.795 | 1.00 | 32.68 | G |
| ATOM | 4995 | C | ILE C | 61 | 77.780 | 22.749 | 37.289 | 1.00 | 31.52 | G |
| ATOM | 4996 | O | ILE C | 61 | 78.360 | 22.707 | 38.368 | 1.00 | 31.59 | G |
| ATOM | 4997 | N | GLU G | 62 | 76.836 | 21.881 | 36.955 | 1.00 | 31.93 | G |
| ATOM | 4998 | CA | GLU G | 62 | 76.443 | 20.861 | 37.907 | 1.00 | 33.23 | G |
| ATOM | 4999 | CB | GLU G | 62 | 75.295 | 20.025 | 37.375 | 1.00 | 32.43 | G |
| ATOM | 5000 | CG | GLU G | 62 | 75.721 | 18.813 | 36.636 | 1.00 | 34.09 | G |
| ATOM | 5001 | CD | GLU G | 62 | 74.649 | 17.762 | 36.649 | 1.00 | 35.90 | G |
| ATOM | 5002 | OE1 | GLU G | 62 | 74.307 | 17.292 | 37.757 | 1.00 | 35.78 | G |
| ATOM | 5003 | OE2 | GLU G | 62 | 74.143 | 17.408 | 35.558 | 1.00 | 37.89 | G |
| ATOM | 5004 | C | GLU G | 62 | 77.595 | 19.939 | 38.278 | 1.00 | 33.56 | G |
| ATOM | 5005 | O | GLU G | 62 | 77.462 | 19.096 | 39.161 | 1.00 | 34.70 | G |
| ATOM | 5006 | N | TRP C | 63 | 78.717 | 20.086 | 37.593 | 1.00 | 33.45 | G |
| ATOM | 5007 | CA | TRP C | 63 | 79.872 | 19.269 | 37.888 | 1.00 | 33.70 | G |
| ATOM | 5008 | CB | TRP C | 63 | 80.670 | 18.991 | 36.622 | 1.00 | 33.66 | G |
| ATOM | 5009 | CG | TRP C | 63 | 81.955 | 18.280 | 36.884 | 1.00 | 33.52 | G |
| ATOM | 5010 | CD2 | TRP C | 63 | 83.211 | 18.876 | 37.231 | 1.00 | 32.28 | G |
| ATOM | 5011 | CE2 | TRP C | 63 | 84.142 | 17.826 | 37.386 | 1.00 | 32.37 | G |
| ATOM | 5012 | CE3 | TRP C | 63 | 83.639 | 20.196 | 37.429 | 1.00 | 31.95 | G |
| ATOM | 5013 | CD1 | TRP C | 63 | 82.167 | 16.935 | 36.846 | 1.00 | 34.55 | G |
| ATOM | 5014 | NE1 | TRP C | 63 | 83.481 | 16.653 | 37.143 | 1.00 | 34.74 | G |
| ATOM | 5015 | CZ2 | TRP C | 63 | 85.476 | 18.049 | 37.726 | 1.00 | 32.07 | G |
| ATOM | 5016 | CZ3 | TRP C | 63 | 84.965 | 20.424 | 37.768 | 1.00 | 31.44 | G |
| ATOM | 5017 | CH2 | TRP C | 63 | 85.871 | 19.351 | 37.914 | 1.00 | 32.65 | G |
| ATOM | 5018 | C | TRP C | 63 | 80.726 | 20.067 | 38.853 | 1.00 | 34.67 | G |
| ATOM | 5019 | O | TRP C | 63 | 81.367 | 19.512 | 39.733 | 1.00 | 36.11 | G |
| ATOM | 5020 | N | ALA C | 64 | 80.737 | 21.380 | 38.686 | 1.00 | 36.01 | C |
| ATOM | 5021 | CA | ALA C | 64 | 81.537 | 22.234 | 39.551 | 1.00 | 38.09 | C |
| ATOM | 5022 | CB | ALA C | 64 | 81.604 | 23.652 | 38.975 | 1.00 | 38.34 | C |
| ATOM | 5023 | C | ALA C | 64 | 80.954 | 22.270 | 40.954 | 1.00 | 38.84 | C |
| ATOM | 5024 | O | ALA C | 64 | 81.653 | 22.010 | 41.928 | 1.00 | 40.17 | C |
| ATOM | 5025 | N | GLU C | 65 | 79.670 | 22.605 | 41.045 | 1.00 | 39.31 | C |
| ATOM | 5026 | CA | GLU C | 65 | 78.981 | 22.675 | 42.322 | 1.00 | 39.42 | C |
| ATOM | 5027 | CB | GLU C | 65 | 77.483 | 22.928 | 42.099 | 1.00 | 37.23 | C |
| ATOM | 5028 | CG | GLU C | 65 | 77.202 | 24.210 | 41.330 | 1.00 | 34.34 | C |
| ATOM | 5029 | CD | GLU C | 65 | 75.724 | 24.533 | 41.190 | 1.00 | 33.67 | C |
| ATOM | 5030 | OE1 | GLU C | 65 | 74.926 | 23.651 | 40.805 | 1.00 | 33.23 | C |
| ATOM | 5031 | OE2 | GLU C | 65 | 75.356 | 25.688 | 41.455 | 1.00 | 33.33 | C |
| ATOM | 5032 | C | GLU C | 65 | 79.196 | 21.376 | 43.101 | 1.00 | 41.07 | C |
| ATOM | 5033 | O | GLU C | 65 | 79.379 | 21.406 | 44.315 | 1.00 | 41.60 | C |
| ATOM | 5034 | N | HIS C | 66 | 79.190 | 20.240 | 42.403 | 1.00 | 42.63 | C |
| ATOM | 5035 | CA | HIS C | 66 | 79.391 | 18.944 | 43.059 | 1.00 | 44.78 | C |
| ATOM | 5036 | CB | HIS C | 66 | 78.988 | 17.796 | 42.119 | 1.00 | 44.20 | C |
| ATOM | 5037 | CG | HIS C | 66 | 79.334 | 16.420 | 42.622 | 1.00 | 44.62 | C |
| ATOM | 5038 | CD2 | HIS C | 66 | 78.548 | 15.363 | 42.937 | 1.00 | 43.92 | C |
| ATOM | 5039 | ND1 | HIS C | 66 | 80.634 | 15.972 | 42.740 | 1.00 | 45.83 | C |
| ATOM | 5040 | CE1 | HIS C | 66 | 80.633 | 14.699 | 43.095 | 1.00 | 43.67 | C |
| ATOM | 5041 | NE2 | HIS C | 66 | 79.379 | 14.305 | 43.221 | 1.00 | 42.62 | C |
| ATOM | 5042 | C | HIS C | 66 | 80.823 | 18.750 | 43.544 | 1.00 | 46.51 | C |
| ATOM | 5043 | O | HIS C | 66 | 81.063 | 18.025 | 44.505 | 1.00 | 47.99 | C |
| ATOM | 5044 | N | HIS C | 67 | 81.781 | 19.393 | 42.894 | 1.00 | 47.66 | C |
| ATOM | 5045 | CA | HIS C | 67 | 83.163 | 19.249 | 43.321 | 1.00 | 49.37 | C |
| ATOM | 5046 | CB | HIS C | 67 | 84.059 | 18.952 | 42.127 | 1.00 | 50.68 | C |
| ATOM | 5047 | CG | HIS C | 67 | 83.964 | 17.541 | 41.652 | 1.00 | 52.46 | C |
| ATOM | 5048 | CD2 | HIS C | 67 | 83.878 | 16.378 | 42.337 | 1.00 | 53.35 | C |
| ATOM | 5049 | ND1 | HIS C | 67 | 83.926 | 17.205 | 40.318 | 1.00 | 54.03 | C |
| ATOM | 5050 | CE1 | HIS C | 67 | 83.815 | 15.894 | 40.199 | 1.00 | 53.47 | C |
| ATOM | 5051 | NE2 | HIS C | 67 | 83.783 | 15.369 | 41.410 | 1.00 | 54.73 | C |
| ATOM | 5052 | C | HIS C | 67 | 83.634 | 20.496 | 44.013 | 1.00 | 50.00 | C |
| ATOM | 5053 | O | HIS C | 67 | 84.831 | 20.793 | 44.022 | 1.00 | 49.65 | C |
| ATOM | 5054 | N | ARG C | 68 | 82.685 | 21.219 | 44.603 | 1.00 | 50.89 | C |
| ATOM | 5055 | CA | ARG C | 68 | 82.996 | 22.468 | 45.295 | 1.00 | 52.52 | C |
| ATOM | 5056 | CB | ARG C | 68 | 81.721 | 23.137 | 45.804 | 1.00 | 51.55 | C |
| ATOM | 5057 | CG | ARG C | 68 | 80.882 | 22.281 | 46.708 | 1.00 | 51.00 | C |
| ATOM | 5058 | CD | ARG C | 68 | 79.609 | 23.021 | 47.114 | 1.00 | 51.32 | C |
| ATOM | 5059 | NE | ARG C | 68 | 78.436 | 22.163 | 46.984 | 1.00 | 50.65 | C |
| ATOM | 5060 | CZ | ARG C | 68 | 78.319 | 20.978 | 47.571 | 1.00 | 49.83 | C |
| ATOM | 5061 | NH1 | ARG C | 68 | 79.299 | 20.514 | 48.331 | 1.00 | 53.30 | C |
| ATOM | 5062 | NH2 | ARG C | 68 | 77.232 | 20.249 | 47.391 | 1.00 | 50.27 | C |
| ATOM | 5063 | C | ARG C | 68 | 83.967 | 22.310 | 46.442 | 1.00 | 53.96 | C |
| ATOM | 5064 | O | ARG C | 68 | 84.685 | 23.243 | 46.784 | 1.00 | 54.35 | C |
| ATOM | 5065 | N | ASP C | 69 | 83.993 | 21.134 | 47.046 | 1.00 | 56.66 | C |
| ATOM | 5066 | CA | ASP C | 69 | 84.905 | 20.921 | 48.150 | 1.00 | 59.49 | C |
| ATOM | 5067 | CB | ASP C | 69 | 84.147 | 20.357 | 49.361 | 1.00 | 58.83 | C |
| ATOM | 5068 | CG | ASP C | 69 | 82.957 | 21.232 | 49.774 | 1.00 | 58.82 | C |
| ATOM | 5069 | OD1 | ASP C | 69 | 83.161 | 22.430 | 50.067 | 1.00 | 57.19 | C |
| ATOM | 5070 | OD2 | ASP C | 69 | 81.814 | 20.718 | 49.806 | 1.00 | 58.80 | C |
| ATOM | 5071 | C | ASP C | 69 | 86.032 | 19.986 | 47.722 | 1.00 | 61.81 | C |
| ATOM | 5072 | O | ASP C | 69 | 87.029 | 19.849 | 48.426 | 1.00 | 64.04 | C |
| ATOM | 5073 | N | SER C | 70 | 85.890 | 19.355 | 46.561 | 1.00 | 62.80 | C |
| ATOM | 5074 | CA | SER C | 70 | 86.928 | 18.445 | 46.089 | 1.00 | 63.95 | C |
| ATOM | 5075 | CB | SER C | 70 | 86.501 | 17.796 | 44.777 | 1.00 | 64.08 | C |
| ATOM | 5076 | OG | SER C | 70 | 85.355 | 16.996 | 44.990 | 1.00 | 65.02 | C |
| ATOM | 5077 | C | SER C | 70 | 88.264 | 19.161 | 45.914 | 1.00 | 64.84 | C |
| ATOM | 5078 | O | SER C | 70 | 88.305 | 20.371 | 45.697 | 1.00 | 65.35 | C |
| ATOM | 5079 | N | ASN C | 71 | 89.360 | 18.418 | 46.029 | 1.00 | 65.33 | C |
| ATOM | 5080 | CA | ASN C | 71 | 90.678 | 19.015 | 45.878 | 1.00 | 65.77 | C |
| ATOM | 5081 | CB | ASN C | 71 | 91.549 | 18.684 | 47.091 | 1.00 | 67.38 | C |
| ATOM | 5082 | CG | ASN C | 71 | 90.805 | 18.874 | 48.409 | 1.00 | 70.03 | C |
| ATOM | 5083 | OD1 | ASN C | 71 | 90.234 | 19.943 | 48.673 | 1.00 | 70.29 | C |
| ATOM | 5084 | ND2 | ASN C | 71 | 90.807 | 17.832 | 49.246 | 1.00 | 70.86 | C |
| ATOM | 5085 | C | ASN C | 71 | 91.311 | 18.488 | 44.603 | 1.00 | 65.48 | C |
| ATOM | 5086 | O | ASN C | 71 | 91.311 | 17.280 | 44.351 | 1.00 | 65.01 | C |
| ATOM | 5087 | N | PHE C | 72 | 91.830 | 19.404 | 43.792 | 1.00 | 64.80 | C |
| ATOM | 5088 | CA | PHE C | 72 | 92.463 | 19.045 | 42.529 | 1.00 | 64.53 | C |
| ATOM | 5089 | CB | PHE C | 72 | 91.585 | 19.473 | 41.342 | 1.00 | 60.50 | C |
| ATOM | 5090 | CG | PHE C | 72 | 90.272 | 18.736 | 41.247 | 1.00 | 55.74 | C |
| ATOM | 5091 | CD1 | PHE C | 72 | 89.068 | 19.433 | 41.235 | 1.00 | 53.23 | C |
| ATOM | 5092 | CD2 | PHE C | 72 | 90.238 | 17.349 | 41.154 | 1.00 | 52.78 | C |
| ATOM | 5093 | CE1 | PHE C | 72 | 87.856 | 18.758 | 41.127 | 1.00 | 51.24 | C |
| ATOM | 5094 | CE2 | PHE C | 72 | 89.027 | 16.672 | 41.047 | 1.00 | 50.62 | C |
| ATOM | 5095 | CZ | PHE C | 72 | 87.837 | 17.378 | 41.034 | 1.00 | 50.12 | C |
| ATOM | 5096 | C | PHE C | 72 | 93.818 | 19.728 | 42.417 | 1.00 | 66.62 | C |
| ATOM | 5097 | O | PHE C | 72 | 93.940 | 20.918 | 42.709 | 1.00 | 67.09 | C |
| ATOM | 5098 | N | PRO C | 73 | 94.853 | 18.981 | 41.988 | 1.00 | 68.12 | C |
| ATOM | 5099 | CD | PRO C | 73 | 94.775 | 17.557 | 41.611 | 1.00 | 68.63 | C |
| ATOM | 5100 | CA | PRO C | 73 | 96.220 | 19.493 | 41.823 | 1.00 | 68.91 | C |
| ATOM | 5101 | CB | PRO C | 73 | 96.935 | 18.342 | 41.118 | 1.00 | 69.55 | C |
| ATOM | 5102 | CG | PRO C | 73 | 96.219 | 17.133 | 41.636 | 1.00 | 69.28 | C |
| ATOM | 5103 | C | PRO C | 73 | 96.257 | 20.778 | 40.990 | 1.00 | 69.17 | C |
| ATOM | 5104 | O | PRO C | 73 | 96.598 | 21.853 | 41.487 | 1.00 | 69.84 | C |
| ATOM | 5105 | N | VAL C | 86 | 90.900 | 7.286 | 36.371 | 1.00 | 88.13 | C |
| ATOM | 5106 | CA | VAL C | 86 | 90.597 | 6.804 | 37.711 | 1.00 | 88.42 | C |
| ATOM | 5107 | CB | VAL C | 86 | 91.894 | 6.677 | 38.553 | 1.00 | 87.81 | C |
| ATOM | 5108 | CG1 | VAL C | 86 | 92.562 | 5.342 | 38.273 | 0.00 | 87.97 | C |
| ATOM | 5109 | CG2 | VAL C | 86 | 92.855 | 7.813 | 38.214 | 1.00 | 86.72 | C |
| ATOM | 5110 | C | VAL C | 86 | 89.573 | 7.685 | 38.445 | 1.00 | 88.62 | C |
| ATOM | 5111 | O | VAL C | 86 | 89.901 | 8.362 | 39.423 | 1.00 | 88.93 | C |
| ATOM | 5112 | N | ASP C | 87 | 88.328 | 7.661 | 37.964 | 1.00 | 88.31 | C |
| ATOM | 5113 | CA | ASP C | 87 | 87.230 | 8.443 | 38.547 | 1.00 | 87.51 | C |
| ATOM | 5114 | CB | ASP C | 87 | 86.568 | 9.310 | 37.475 | 0.00 | 88.63 | C |
| ATOM | 5115 | CG | ASP C | 87 | 87.538 | 10.229 | 36.783 | 1.00 | 89.65 | C |
| ATOM | 5116 | OD1 | ASP C | 87 | 87.832 | 11.313 | 37.331 | 1.00 | 90.42 | C |
| ATOM | 5117 | OD2 | ASP C | 87 | 88.011 | 9.859 | 35.688 | 1.00 | 89.70 | C |
| ATOM | 5118 | C | ASP C | 87 | 86.150 | 7.559 | 39.166 | 1.00 | 86.41 | C |
| ATOM | 5119 | O | ASP C | 87 | 86.396 | 6.413 | 39.543 | 0.00 | 86.24 | C |
| ATOM | 5120 | N | SER C | 88 | 84.944 | 8.110 | 39.245 | 1.00 | 84.26 | C |
| ATOM | 5121 | CA | SER C | 88 | 83.803 | 7.402 | 39.800 | 1.00 | 82.05 | C |
| ATOM | 5122 | CB | SER C | 88 | 83.872 | 7.398 | 41.324 | 1.00 | 81.81 | C |
| ATOM | 5123 | OG | SER C | 88 | 82.634 | 6.987 | 41.874 | 1.00 | 81.31 | C |
| ATOM | 5124 | C | SER C | 88 | 82.508 | 8.067 | 39.360 | 1.00 | 80.69 | C |
| ATOM | 5125 | O | SER C | 88 | 81.919 | 7.704 | 38.342 | 1.00 | 81.28 | C |
| ATOM | 5126 | N | TRP C | 89 | 82.066 | 9.042 | 40.144 | 1.00 | 77.91 | C |
| ATOM | 5127 | CA | TRP C | 89 | 80.848 | 9.780 | 39.848 | 1.00 | 74.10 | C |
| ATOM | 5128 | CB | TRP C | 89 | 80.646 | 10.859 | 40.905 | 1.00 | 73.48 | C |
| ATOM | 5129 | CG | TRP C | 89 | 79.446 | 11.708 | 40.702 | 1.00 | 72.20 | C |
| ATOM | 5130 | CD2 | TRP C | 89 | 79.428 | 13.044 | 40.197 | 1.00 | 71.86 | C |
| ATOM | 5131 | CE2 | TRP C | 89 | 78.088 | 13.476 | 40.209 | 1.00 | 71.45 | C |
| ATOM | 5132 | CE3 | TRP C | 89 | 80.418 | 13.919 | 39.735 | 1.00 | 71.66 | C |
| ATOM | 5133 | CD1 | TRP C | 89 | 78.153 | 11.388 | 40.988 | 1.00 | 71.28 | C |
| ATOM | 5134 | NE1 | TRP C | 89 | 77.329 | 12.446 | 40.698 | 1.00 | 70.67 | C |
| ATOM | 5135 | CZ2 | TRP C | 89 | 77.711 | 14.750 | 39.780 | 1.00 | 71.55 | C |
| ATOM | 5136 | CZ3 | TRP C | 89 | 80.043 | 15.184 | 39.308 | 1.00 | 71.26 | C |
| ATOM | 5137 | CH2 | TRP C | 89 | 78.701 | 15.587 | 39.334 | 1.00 | 71.21 | C |
| ATOM | 5138 | C | TRP C | 89 | 80.995 | 10.415 | 38.473 | 1.00 | 72.28 | C |
| ATOM | 5139 | O | TRP C | 89 | 80.021 | 10.567 | 37.740 | 1.00 | 72.30 | C |
| ATOM | 5140 | N | ASP C | 90 | 82.229 | 10.779 | 38.136 | 1.00 | 70.00 | C |
| ATOM | 5141 | CA | ASP C | 90 | 82.543 | 11.389 | 36.848 | 1.00 | 67.79 | C |
| ATOM | 5142 | CB | ASP C | 90 | 84.011 | 11.810 | 36.808 | 1.00 | 64.52 | C |
| ATOM | 5143 | CG | ASP C | 90 | 84.300 | 13.010 | 37.685 | 1.00 | 61.24 | C |
| ATOM | 5144 | OD1 | ASP C | 90 | 83.453 | 13.363 | 38.530 | 1.00 | 59.13 | C |
| ATOM | 5145 | OD2 | ASP C | 90 | 85.388 | 13.594 | 37.533 | 1.00 | 58.53 | C |
| ATOM | 5146 | C | ASP C | 90 | 82.261 | 10.436 | 35.691 | 1.00 | 67.74 | C |
| ATOM | 5147 | O | ASP C | 90 | 81.888 | 10.868 | 34.597 | 1.00 | 68.39 | C |
| ATOM | 5148 | N | ARG C | 91 | 82.460 | 9.142 | 35.926 | 1.00 | 67.23 | C |
| ATOM | 5149 | CA | ARG C | 91 | 82.197 | 8.140 | 34.895 | 1.00 | 66.43 | C |
| ATOM | 5150 | CB | ARG C | 91 | 82.671 | 6.755 | 35.345 | 0.00 | 67.74 | C |
| ATOM | 5151 | CG | ARG C | 91 | 82.250 | 5.602 | 34.429 | 0.00 | 69.73 | C |
| ATOM | 5152 | CD | ARG C | 91 | 82.769 | 5.731 | 32.997 | 0.00 | 71.43 | C |
| ATOM | 5153 | NE | ARG C | 91 | 81.880 | 6.509 | 32.132 | 1.00 | 73.30 | C |
| ATOM | 5154 | CZ | ARG C | 91 | 81.888 | 6.448 | 30.803 | 1.00 | 73.96 | C |
| ATOM | 5155 | NH1 | ARG C | 91 | 82.739 | 5.645 | 30.176 | 1.00 | 75.13 | C |
| ATOM | 5156 | NH2 | ARG C | 91 | 81.038 | 7.180 | 30.098 | 1.00 | 73.71 | C |
| ATOM | 5157 | C | ARG C | 91 | 80.707 | 8.091 | 34.580 | 1.00 | 64.28 | C |
| ATOM | 5158 | O | ARG C | 91 | 80.322 | 8.005 | 33.416 | 1.00 | 64.65 | C |
| ATOM | 5159 | N | GLU C | 92 | 79.871 | 8.156 | 35.611 | 1.00 | 61.21 | C |
| ATOM | 5160 | CA | GLU C | 92 | 78.434 | 8.111 | 35.398 | 1.00 | 59.24 | C |
| ATOM | 5161 | CB | GLU C | 92 | 77.709 | 7.733 | 36.691 | 1.00 | 61.28 | C |
| ATOM | 5162 | CG | GLU C | 92 | 76.200 | 7.577 | 36.529 | 1.00 | 63.29 | C |
| ATOM | 5163 | CD | GLU C | 92 | 75.816 | 6.698 | 35.345 | 1.00 | 64.91 | C |
| ATOM | 5164 | OE1 | GLU C | 92 | 76.430 | 5.618 | 35.171 | 1.00 | 65.27 | C |
| ATOM | 5165 | OE2 | GLU C | 92 | 74.888 | 7.087 | 34.599 | 1.00 | 65.86 | C |
| ATOM | 5166 | C | GLU C | 92 | 77.947 | 9.463 | 34.908 | 1.00 | 56.10 | C |
| ATOM | 5167 | O | GLU C | 92 | 76.846 | 9.587 | 34.379 | 1.00 | 55.26 | C |
| ATOM | 5168 | N | PHE C | 93 | 78.786 | 10.473 | 35.088 | 1.00 | 52.97 | C |
| ATOM | 5169 | CA | PHE C | 93 | 78.470 | 11.828 | 34.669 | 1.00 | 49.46 | C |
| ATOM | 5170 | CB | PHE C | 93 | 79.271 | 12.821 | 35.504 | 1.00 | 47.72 | C |
| ATOM | 5171 | CG | PHE C | 93 | 79.168 | 14.237 | 35.031 | 1.00 | 46.31 | C |
| ATOM | 5172 | CD1 | PHE C | 93 | 77.975 | 14.936 | 35.134 | 1.00 | 46.80 | C |
| ATOM | 5173 | CD2 | PHE C | 93 | 80.274 | 14.880 | 34.487 | 1.00 | 46.30 | C |
| ATOM | 5174 | GE1 | PHE C | 93 | 77.881 | 16.260 | 34.698 | 1.00 | 46.57 | C |
| ATOM | 5175 | CE2 | PHE C | 93 | 80.194 | 16.200 | 34.048 | 1.00 | 45.89 | C |
| ATOM | 5176 | CZ | PHE C | 93 | 78.995 | 16.891 | 34.155 | 1.00 | 46.39 | C |
| ATOM | 5177 | C | PHE C | 93 | 78.805 | 12.007 | 33.195 | 1.00 | 48.24 | C |
| ATOM | 5178 | O | PHE C | 93 | 78.194 | 12.829 | 32.512 | 1.00 | 48.29 | C |
| ATOM | 5179 | N | LEU C | 94 | 79.777 | 11.241 | 32.706 | 1.00 | 46.95 | C |
| ATOM | 5180 | CA | LEU C | 94 | 80.181 | 11.334 | 31.304 | 1.00 | 46.69 | C |
| ATOM | 5181 | CB | LEU C | 94 | 81.711 | 11.421 | 31.201 | 1.00 | 43.72 | C |
| ATOM | 5182 | CG | LEU C | 94 | 82.347 | 12.602 | 31.931 | 1.00 | 42.20 | C |
| ATOM | 5183 | CD1 | LEU C | 94 | 83.827 | 12.571 | 31.708 | 1.00 | 42.01 | C |
| ATOM | 5184 | CD2 | LEU C | 94 | 81.761 | 13.918 | 31.444 | 1.00 | 40.79 | C |
| ATOM | 5185 | C | LEU C | 94 | 79.657 | 10.163 | 30.470 | 1.00 | 46.61 | C |
| ATOM | 5186 | O | LEU C | 94 | 80.084 | 9.950 | 29.331 | 1.00 | 47.53 | C |
| ATOM | 5187 | N | LYS C | 95 | 78.721 | 9.416 | 31.044 | 1.00 | 46.24 | C |
| ATOM | 5188 | CA | LYS C | 95 | 78.123 | 8.260 | 30.382 | 1.00 | 46.42 | C |
| ATOM | 5189 | CB | LYS C | 95 | 77.447 | 7.372 | 31.436 | 1.00 | 48.23 | C |
| ATOM | 5190 | CG | LYS C | 95 | 77.259 | 5.915 | 31.045 | 1.00 | 48.85 | C |
| ATOM | 5191 | CD | LYS C | 95 | 75.858 | 5.649 | 30.493 | 1.00 | 50.94 | C |
| ATOM | 5192 | CE | LYS C | 95 | 74.749 | 5.845 | 31.556 | 1.00 | 51.20 | C |
| ATOM | 5193 | NZ | LYS C | 95 | 74.537 | 7.259 | 32.014 | 1.00 | 49.42 | C |
| ATOM | 5194 | C | LYS C | 95 | 77.103 | 8.758 | 29.359 | 1.00 | 45.40 | C |
| ATOM | 5195 | O | LYS C | 95 | 76.000 | 8.222 | 29.234 | 1.00 | 45.79 | C |
| ATOM | 5196 | N | VAL C | 96 | 77.495 | 9.797 | 28.631 | 1.00 | 42.97 | C |
| ATOM | 5197 | CA | VAL C | 96 | 76.648 | 10.418 | 27.627 | 1.00 | 39.32 | C |
| ATOM | 5198 | CB | VAL C | 96 | 76.748 | 11.930 | 27.759 | 1.00 | 38.19 | C |
| ATOM | 5199 | CG1 | VAL C | 96 | 76.296 | 12.335 | 29.143 | 1.00 | 37.83 | C |
| ATOM | 5200 | CG2 | VAL C | 96 | 78.182 | 12.369 | 27.543 | 1.00 | 37.61 | C |
| ATOM | 5201 | C | VAL C | 96 | 77.069 | 9.973 | 26.224 | 1.00 | 39.45 | C |
| ATOM | 5202 | O | VAL C | 96 | 77.934 | 9.110 | 26.077 | 1.00 | 39.51 | C |
| ATOM | 5203 | N | ASP C | 97 | 76.448 | 10.545 | 25.197 | 1.00 | 38.96 | C |
| ATOM | 5204 | CA | ASP C | 97 | 76.776 | 10.194 | 23.820 | 1.00 | 38.45 | C |
| ATOM | 5205 | CB | ASP C | 97 | 75.596 | 10.485 | 22.907 | 1.00 | 40.12 | C |
| ATOM | 5206 | CG | ASP C | 97 | 75.212 | 11.951 | 22.907 | 1.00 | 41.57 | C |
| ATOM | 5207 | OD1 | ASP C | 97 | 76.061 | 12.785 | 23.289 | 1.00 | 41.15 | C |
| ATOM | 5208 | OD2 | ASP C | 97 | 74.066 | 12.271 | 22.516 | 1.00 | 42.19 | C |
| ATOM | 5209 | C | ASP C | 97 | 77.986 | 10.982 | 23.328 | 1.00 | 38.55 | C |
| ATOM | 5210 | O | ASP C | 97 | 78.331 | 12.014 | 23.893 | 1.00 | 37.77 | C |
| ATOM | 5211 | N | GLN C | 98 | 78.612 | 10.505 | 22.259 | 1.00 | 39.05 | C |
| ATOM | 5212 | CA | GLN C | 98 | 79.794 | 11.157 | 21.717 | 1.00 | 38.40 | C |
| ATOM | 5213 | CB | GLN C | 98 | 80.225 | 10.485 | 20.415 | 1.00 | 38.10 | C |
| ATOM | 5214 | CG | GLN C | 98 | 80.605 | 9.023 | 20.544 | 1.00 | 36.09 | C |
| ATOM | 5215 | CD | GLN C | 98 | 81.275 | 8.497 | 19.298 | 1.00 | 36.91 | C |
| ATOM | 5216 | OE1 | GLN C | 98 | 81.239 | 9.130 | 18.236 | 1.00 | 36.92 | C |
| ATOM | 5217 | NE2 | GLN C | 98 | 81.881 | 7.324 | 19.412 | 1.00 | 36.42 | C |
| ATOM | 5218 | C | GLN C | 98 | 79.650 | 12.653 | 21.470 | 1.00 | 39.08 | C |
| ATOM | 5219 | O | GLN C | 98 | 80.599 | 13.401 | 21.692 | 1.00 | 40.00 | C |
| ATOM | 5220 | N | GLU C | 99 | 78.486 | 13.088 | 21.001 | 1.00 | 39.22 | C |
| ATOM | 5221 | CA | GLU C | 99 | 78.271 | 14.502 | 20.726 | 1.00 | 40.50 | C |
| ATOM | 5222 | CB | GLU C | 99 | 76.891 | 14.717 | 20.126 | 1.00 | 42.47 | C |
| ATOM | 5223 | CG | GLU C | 99 | 76.918 | 15.343 | 18.756 | 1.00 | 45.54 | C |
| ATOM | 5224 | CD | GLU C | 99 | 77.806 | 16.577 | 18.697 | 1.00 | 47.73 | C |
| ATOM | 5225 | OE1 | GLU C | 99 | 77.552 | 17.541 | 19.462 | 1.00 | 47.01 | C |
| ATOM | 5226 | OE2 | GLU C | 99 | 78.757 | 16.577 | 17.881 | 1.00 | 47.67 | C |
| ATOM | 5227 | C | GLU C | 99 | 78.387 | 15.335 | 21.990 | 1.00 | 41.46 | C |
| ATOM | 5228 | O | GLU C | 99 | 78.980 | 16.418 | 21.993 | 1.00 | 41.51 | C |
| ATOM | 5229 | N | MSE C | 100 | 77.788 | 14.831 | 23.061 | 1.00 | 41.97 | C |
| ATOM | 5230 | CA | MSE C | 100 | 77.818 | 15.518 | 24.335 | 1.00 | 41.69 | C |
| ATOM | 5231 | CB | MSE C | 100 | 76.885 | 14.824 | 25.333 | 1.00 | 45.69 | C |
| ATOM | 5232 | CG | MSE C | 100 | 76.914 | 15.403 | 26.735 | 1.00 | 51.76 | C |
| ATOM | 5233 | SE | MSE C | 100 | 76.090 | 17.140 | 26.925 | 1.00 | 62.68 | C |
| ATOM | 5234 | CE | MSE C | 100 | 77.565 | 18.303 | 26.510 | 1.00 | 57.90 | C |
| ATOM | 5235 | C | MSE C | 100 | 79.243 | 15.546 | 24.867 | 1.00 | 39.87 | C |
| ATOM | 5236 | O | MSE C | 100 | 79.810 | 16.626 | 25.037 | 1.00 | 39.79 | C |
| ATOM | 5237 | N | LEU C | 101 | 79.835 | 14.376 | 25.115 | 1.00 | 37.39 | C |
| ATOM | 5238 | CA | LEU C | 101 | 81.199 | 14.341 | 25.651 | 1.00 | 34.99 | C |
| ATOM | 5239 | CB | LEU C | 101 | 81.809 | 12.938 | 25.539 | 1.00 | 33.34 | C |
| ATOM | 5240 | OG | LEU C | 101 | 83.130 | 12.730 | 26.289 | 1.00 | 30.53 | C |
| ATOM | 5241 | OD1 | LEU C | 101 | 82.896 | 12.845 | 27.780 | 1.00 | 31.52 | C |
| ATOM | 5242 | OD2 | LEU C | 101 | 83.684 | 11.363 | 25.965 | 1.00 | 30.51 | C |
| ATOM | 5243 | C | LEU C | 101 | 82.067 | 15.347 | 24.904 | 1.00 | 34.53 | C |
| ATOM | 5244 | O | LEU C | 101 | 82.753 | 16.184 | 25.516 | 1.00 | 34.23 | C |
| ATOM | 5245 | N | TYR C | 102 | 82.011 | 15.274 | 23.577 | 1.00 | 31.29 | C |
| ATOM | 5246 | CA | TYR C | 102 | 82.770 | 16.179 | 22.716 | 1.00 | 28.61 | C |
| ATOM | 5247 | CB | TYR C | 102 | 82.335 | 15.955 | 21.258 | 1.00 | 27.71 | C |
| ATOM | 5248 | CG | TYR C | 102 | 82.876 | 16.976 | 20.288 | 1.00 | 26.47 | C |
| ATOM | 5249 | CD1 | TYR C | 102 | 84.220 | 16.970 | 19.896 | 1.00 | 24.67 | C |
| ATOM | 5250 | CE1 | TYR C | 102 | 84.708 | 17.930 | 19.005 | 1.00 | 23.69 | C |
| ATOM | 5251 | CD2 | TYR C | 102 | 82.040 | 17.960 | 19.774 | 1.00 | 25.15 | C |
| ATOM | 5252 | CE2 | TYR C | 102 | 82.505 | 18.913 | 18.899 | 1.00 | 23.80 | C |
| ATOM | 5253 | CZ | TYR C | 102 | 83.827 | 18.899 | 18.512 | 1.00 | 23.80 | C |
| ATOM | 5254 | OH | TYR C | 102 | 84.237 | 19.853 | 17.626 | 1.00 | 21.51 | C |
| ATOM | 5255 | C | TYR C | 102 | 82.572 | 17.659 | 23.131 | 1.00 | 27.38 | C |
| ATOM | 5256 | O | TYR C | 102 | 83.536 | 18.383 | 23.433 | 1.00 | 23.78 | C |
| ATOM | 5257 | N | GLU C | 103 | 81.314 | 18.092 | 23.155 | 1.00 | 28.53 | C |
| ATOM | 5258 | CA | GLU C | 103 | 80.979 | 19.462 | 23.532 | 1.00 | 29.26 | C |
| ATOM | 5259 | CB | GLU C | 103 | 79.483 | 19.706 | 23.342 | 1.00 | 30.52 | C |
| ATOM | 5260 | CG | GLU C | 103 | 79.063 | 19.574 | 21.888 | 1.00 | 33.75 | C |
| ATOM | 5261 | CD | GLU C | 103 | 77.781 | 20.318 | 21.565 | 1.00 | 36.95 | C |
| ATOM | 5262 | OE1 | GLU C | 103 | 77.729 | 21.549 | 21.820 | 1.00 | 41.36 | C |
| ATOM | 5263 | OE2 | GLU C | 103 | 76.832 | 19.682 | 21.053 | 1.00 | 35.81 | C |
| ATOM | 5264 | C | GLU C | 103 | 81.397 | 19.817 | 24.954 | 1.00 | 27.19 | C |
| ATOM | 5265 | O | GLU C | 103 | 81.801 | 20.950 | 25.217 | 1.00 | 27.16 | C |
| ATOM | 5266 | N | ILE C | 104 | 81.317 | 18.855 | 25.870 | 1.00 | 26.37 | C |
| ATOM | 5267 | CA | ILE C | 104 | 81.717 | 19.114 | 27.254 | 1.00 | 25.70 | C |
| ATOM | 5268 | CB | ILE C | 104 | 81.356 | 17.913 | 28.167 | 1.00 | 25.25 | C |
| ATOM | 5269 | CG2 | ILE C | 104 | 82.091 | 17.986 | 29.485 | 1.00 | 25.29 | C |
| ATOM | 5270 | CG1 | ILE C | 104 | 79.859 | 17.935 | 28.450 | 1.00 | 25.32 | C |
| ATOM | 5271 | OD1 | ILE C | 104 | 79.346 | 16.709 | 29.127 | 1.00 | 25.12 | C |
| ATOM | 5272 | C | ILE C | 104 | 83.210 | 19.423 | 27.310 | 1.00 | 24.54 | C |
| ATOM | 5273 | O | ILE C | 104 | 83.614 | 20.452 | 27.848 | 1.00 | 22.76 | C |
| ATOM | 5274 | N | ILE C | 105 | 84.011 | 18.528 | 26.731 | 1.00 | 24.67 | C |
| ATOM | 5275 | CA | ILE C | 105 | 85.464 | 18.687 | 26.673 | 1.00 | 23.37 | C |
| ATOM | 5276 | CB | ILE C | 105 | 86.120 | 17.563 | 25.834 | 1.00 | 22.40 | C |
| ATOM | 5277 | CG2 | ILE C | 105 | 87.524 | 17.985 | 25.410 | 1.00 | 22.41 | C |
| ATOM | 5278 | CG1 | ILE C | 105 | 86.143 | 16.249 | 26.630 | 1.00 | 22.74 | C |
| ATOM | 5279 | CD1 | ILE C | 105 | 86.616 | 15.035 | 25.826 | 1.00 | 19.84 | C |
| ATOM | 5280 | C | ILE C | 105 | 85.844 | 20.038 | 26.057 | 1.00 | 24.11 | C |
| ATOM | 5281 | O | ILE C | 105 | 86.885 | 20.601 | 26.383 | 1.00 | 23.21 | C |
| ATOM | 5282 | N | LEU C | 106 | 85.007 | 20.568 | 25.170 | 1.00 | 25.59 | C |
| ATOM | 5283 | CA | LEU C | 106 | 85.341 | 21.845 | 24.546 | 1.00 | 26.22 | C |
| ATOM | 5284 | CB | LEU C | 106 | 84.550 | 22.060 | 23.251 | 1.00 | 24.35 | C |
| ATOM | 5285 | CG | LEU C | 106 | 84.955 | 21.288 | 21.982 | 1.00 | 24.05 | C |
| ATOM | 5286 | CD1 | LEU C | 106 | 84.041 | 21.702 | 20.824 | 1.00 | 19.50 | C |
| ATOM | 5287 | CD2 | LEU C | 106 | 86.411 | 21.582 | 21.631 | 1.00 | 19.81 | C |
| ATOM | 5288 | C | LEU C | 106 | 85.051 | 22.972 | 25.504 | 1.00 | 27.00 | C |
| ATOM | 5289 | O | LEU C | 106 | 85.876 | 23.871 | 25.700 | 1.00 | 28.04 | C |
| ATOM | 5290 | N | ALA C | 107 | 83.870 | 22.908 | 26.107 | 1.00 | 27.32 | C |
| ATOM | 5291 | CA | ALA C | 107 | 83.432 | 23.921 | 27.054 | 1.00 | 27.07 | C |
| ATOM | 5292 | CB | ALA C | 107 | 82.044 | 23.574 | 27.562 | 1.00 | 27.09 | C |
| ATOM | 5293 | C | ALA C | 107 | 84.417 | 23.967 | 28.210 | 1.00 | 26.97 | C |
| ATOM | 5294 | O | ALA C | 107 | 84.759 | 25.040 | 28.720 | 1.00 | 25.71 | C |
| ATOM | 5295 | N | ALA C | 108 | 84.866 | 22.786 | 28.621 | 1.00 | 27.46 | C |
| ATOM | 5296 | CA | ALA C | 108 | 85.809 | 22.688 | 29.726 | 1.00 | 28.55 | C |
| ATOM | 5297 | CB | ALA C | 108 | 86.059 | 21.230 | 30.069 | 1.00 | 27.60 | C |
| ATOM | 5298 | C | ALA C | 108 | 87.119 | 23.383 | 29.363 | 1.00 | 29.01 | C |
| ATOM | 5299 | O | ALA C | 108 | 87.595 | 24.264 | 30.085 | 1.00 | 29.74 | C |
| ATOM | 5300 | N | ASN C | 109 | 87.684 | 22.988 | 28.227 | 1.00 | 28.31 | C |
| ATOM | 5301 | CA | ASN C | 109 | 88.934 | 23.554 | 27.723 | 1.00 | 27.27 | C |
| ATOM | 5302 | CB | ASN C | 109 | 89.213 | 22.988 | 26.323 | 1.00 | 27.05 | C |
| ATOM | 5303 | CG | ASN C | 109 | 90.436 | 23.594 | 25.666 | 1.00 | 24.65 | C |
| ATOM | 5304 | OD1 | ASN C | 109 | 91.542 | 23.472 | 26.167 | 1.00 | 22.66 | C |
| ATOM | 5305 | ND2 | ASN C | 109 | 90.236 | 24.235 | 24.517 | 1.00 | 24.60 | C |
| ATOM | 5306 | C | ASN C | 109 | 88.848 | 25.074 | 27.661 | 1.00 | 27.03 | C |
| ATOM | 5307 | O | ASN C | 109 | 89.755 | 25.770 | 28.109 | 1.00 | 28.21 | C |
| ATOM | 5308 | N | TYR C | 110 | 87.743 | 25.575 | 27.118 | 1.00 | 26.52 | C |
| ATOM | 5309 | CA | TYR C | 110 | 87.528 | 27.003 | 26.969 | 1.00 | 26.57 | C |
| ATOM | 5310 | CE | TYR C | 110 | 86.268 | 27.267 | 26.139 | 1.00 | 25.48 | C |
| ATOM | 5311 | CG | TYR C | 110 | 85.938 | 28.734 | 26.018 | 1.00 | 24.82 | C |
| ATOM | 5312 | CD1 | TYR C | 110 | 86.780 | 29.596 | 25.317 | 1.00 | 26.81 | C |
| ATOM | 5313 | CE1 | TYR C | 110 | 86.543 | 30.968 | 25.273 | 1.00 | 26.16 | C |
| ATOM | 5314 | CD2 | TYR C | 110 | 84.838 | 29.282 | 26.669 | 1.00 | 24.41 | C |
| ATOM | 5315 | CE2 | TYR C | 110 | 84.596 | 30.658 | 26.628 | 1.00 | 24.83 | C |
| ATOM | 5316 | CZ | TYR C | 110 | 85.455 | 31.486 | 25.930 | 1.00 | 24.87 | C |
| ATOM | 5317 | OH | TYR C | 110 | 85.234 | 32.835 | 25.889 | 1.00 | 25.53 | C |
| ATOM | 5318 | C | TYR C | 110 | 87.371 | 27.682 | 28.308 | 1.00 | 27.74 | C |
| ATOM | 5319 | O | TYR C | 110 | 87.907 | 28.767 | 28.546 | 1.00 | 27.37 | C |
| ATOM | 5320 | N | LEU C | 111 | 86.610 | 27.040 | 29.178 | 1.00 | 28.23 | C |
| ATOM | 5321 | CA | LEU C | 111 | 86.348 | 27.593 | 30.485 | 1.00 | 28.18 | C |
| ATOM | 5322 | CE | LEU C | 111 | 85.067 | 26.958 | 31.031 | 1.00 | 25.97 | C |
| ATOM | 5323 | CG | LEU C | 111 | 83.874 | 27.827 | 31.434 | 1.00 | 24.04 | C |
| ATOM | 5324 | CD1 | LEU C | 111 | 83.755 | 29.132 | 30.657 | 1.00 | 19.70 | C |
| ATOM | 5325 | CD2 | LEU C | 111 | 82.653 | 26.963 | 31.273 | 1.00 | 24.33 | C |
| ATOM | 5326 | C | LEU C | 111 | 87.547 | 27.373 | 31.410 | 1.00 | 29.42 | C |
| ATOM | 5327 | O | LEU C | 111 | 87.524 | 27.765 | 32.578 | 1.00 | 31.45 | C |
| ATOM | 5328 | N | ASN C | 112 | 88.601 | 26.761 | 30.875 | 1.00 | 29.81 | C |
| ATOM | 5329 | CA | ASN C | 112 | 89.825 | 26.513 | 31.640 | 1.00 | 31.10 | C |
| ATOM | 5330 | CB | ASN C | 112 | 90.498 | 27.835 | 32.006 | 1.00 | 31.09 | C |
| ATOM | 5331 | CG | ASN C | 112 | 91.974 | 27.673 | 32.371 | 1.00 | 32.09 | C |
| ATOM | 5332 | OD1 | ASN C | 112 | 92.448 | 26.579 | 32.719 | 1.00 | 29.20 | C |
| ATOM | 5333 | ND2 | ASN C | 112 | 92.708 | 28.783 | 32.300 | 1.00 | 33.07 | C |
| ATOM | 5334 | C | ASN C | 112 | 89.563 | 25.740 | 32.927 | 1.00 | 32.17 | C |
| ATOM | 5335 | O | ASN C | 112 | 89.805 | 26.237 | 34.030 | 1.00 | 33.29 | C |
| ATOM | 5336 | N | ILE C | 113 | 89.055 | 24.527 | 32.789 | 1.00 | 31.11 | C |
| ATOM | 5337 | CA | ILE C | 113 | 88.798 | 23.717 | 33.954 | 1.00 | 30.18 | C |
| ATOM | 5338 | CE | ILE C | 113 | 87.322 | 23.467 | 34.163 | 1.00 | 30.05 | C |
| ATOM | 5339 | CG2 | ILE C | 113 | 87.131 | 22.660 | 35.442 | 1.00 | 28.92 | C |
| ATOM | 5340 | CG1 | ILE C | 113 | 86.564 | 24.789 | 34.234 | 1.00 | 28.96 | C |
| ATOM | 5341 | CD1 | ILE C | 113 | 85.076 | 24.575 | 34.283 | 1.00 | 28.52 | C |
| ATOM | 5342 | C | ILE C | 113 | 89.473 | 22.384 | 33.751 | 1.00 | 30.85 | C |
| ATOM | 5343 | O | ILE C | 113 | 88.834 | 21.400 | 33.387 | 1.00 | 29.72 | C |
| ATOM | 5344 | N | LYS C | 114 | 90.778 | 22.368 | 33.998 | 1.00 | 31.38 | C |
| ATOM | 5345 | CA | LYS C | 114 | 91.595 | 21.173 | 33.838 | 1.00 | 31.36 | C |
| ATOM | 5346 | CB | LYS C | 114 | 93.003 | 21.440 | 34.383 | 1.00 | 32.37 | C |
| ATOM | 5347 | CG | LYS C | 114 | 93.901 | 20.219 | 34.344 | 1.00 | 34.25 | C |
| ATOM | 5348 | CD | LYS C | 114 | 95.372 | 20.555 | 34.550 | 1.00 | 35.39 | C |
| ATOM | 5349 | CE | LYS C | 114 | 96.190 | 19.289 | 34.455 | 1.00 | 36.88 | C |
| ATOM | 5350 | NZ | LYS C | 114 | 95.863 | 18.587 | 33.190 | 1.00 | 40.06 | C |
| ATOM | 5351 | C | LYS C | 114 | 91.035 | 19.876 | 34.437 | 1.00 | 30.61 | C |
| ATOM | 5352 | O | LYS C | 114 | 91.054 | 18.841 | 33.785 | 1.00 | 30.70 | C |
| ATOM | 5353 | N | PRO C | 115 | 90.530 | 19.916 | 35.681 | 1.00 | 30.70 | C |
| ATOM | 5354 | CD | PRO C | 115 | 90.401 | 21.106 | 36.545 | 1.00 | 31.09 | C |
| ATOM | 5355 | CA | PRO C | 115 | 89.970 | 18.719 | 36.338 | 1.00 | 30.04 | C |
| ATOM | 5356 | CB | PRO C | 115 | 89.365 | 19.276 | 37.626 | 1.00 | 30.45 | C |
| ATOM | 5357 | CG | PRO C | 115 | 90.220 | 20.489 | 37.912 | 1.00 | 31.85 | C |
| ATOM | 5358 | C | PRO C | 115 | 88.914 | 18.030 | 35.482 | 1.00 | 29.80 | C |
| ATOM | 5359 | O | PRO C | 115 | 88.935 | 16.806 | 35.295 | 1.00 | 30.05 | C |
| ATOM | 5360 | N | LEU C | 116 | 87.981 | 18.833 | 34.974 | 1.00 | 29.91 | C |
| ATOM | 5361 | CA | LEU C | 116 | 86.904 | 18.330 | 34.114 | 1.00 | 29.38 | C |
| ATOM | 5362 | CB | LEU C | 116 | 85.797 | 19.376 | 33.980 | 1.00 | 27.77 | C |
| ATOM | 5363 | CG | LEU C | 116 | 84.628 | 18.909 | 33.138 | 1.00 | 27.38 | C |
| ATOM | 5364 | CD1 | LEU C | 116 | 84.043 | 17.676 | 33.768 | 1.00 | 27.48 | C |
| ATOM | 5365 | CD2 | LEU C | 116 | 83.597 | 20.023 | 33.027 | 1.00 | 27.97 | C |
| ATOM | 5366 | C | LEU C | 116 | 87.464 | 17.974 | 32.731 | 1.00 | 28.24 | C |
| ATOM | 5367 | O | LEU C | 116 | 87.042 | 17.003 | 32.107 | 1.00 | 28.34 | C |
| ATOM | 5368 | N | LEU C | 117 | 88.433 | 18.756 | 32.272 | 1.00 | 26.39 | C |
| ATOM | 5369 | CA | LEU C | 117 | 89.051 | 18.494 | 30.993 | 1.00 | 26.69 | C |
| ATOM | 5370 | CB | LEU C | 117 | 90.112 | 19.547 | 30.701 | 1.00 | 24.99 | C |
| ATOM | 5371 | CG | LEU C | 117 | 90.893 | 19.362 | 29.397 | 1.00 | 24.42 | C |
| ATOM | 5372 | CD1 | LEU C | 117 | 89.943 | 19.112 | 28.225 | 1.00 | 23.60 | C |
| ATOM | 5373 | CD2 | LEU C | 117 | 91.738 | 20.593 | 29.154 | 1.00 | 22.70 | C |
| ATOM | 5374 | C | LEU C | 117 | 89.680 | 17.100 | 30.981 | 1.00 | 28.34 | C |
| ATOM | 5375 | O | LEU C | 117 | 89.397 | 16.288 | 30.093 | 1.00 | 29.22 | C |
| ATOM | 5376 | N | ASP C | 118 | 90.528 | 16.826 | 31.973 | 1.00 | 29.30 | C |
| ATOM | 5377 | CA | ASP C | 118 | 91.207 | 15.532 | 32.105 | 1.00 | 29.52 | C |
| ATOM | 5378 | CB | ASP C | 118 | 92.117 | 15.538 | 33.347 | 1.00 | 32.96 | C |
| ATOM | 5379 | CG | ASP C | 118 | 93.158 | 16.668 | 33.316 | 1.00 | 36.46 | C |
| ATOM | 5380 | OD1 | ASP C | 118 | 93.138 | 17.470 | 32.353 | 1.00 | 37.36 | C |
| ATOM | 5381 | OD2 | ASP C | 118 | 93.990 | 16.757 | 34.253 | 1.00 | 36.80 | C |
| ATOM | 5382 | C | ASP C | 118 | 90.213 | 14.365 | 32.186 | 1.00 | 28.84 | C |
| ATOM | 5383 | O | ASP C | 118 | 90.289 | 13.418 | 31.391 | 1.00 | 28.39 | C |
| ATOM | 5384 | N | ALA C | 119 | 89.288 | 14.419 | 33.142 | 1.00 | 26.34 | C |
| ATOM | 5385 | CA | ALA C | 119 | 88.293 | 13.348 | 33.262 | 1.00 | 25.58 | C |
| ATOM | 5386 | CB | ALA C | 119 | 87.208 | 13.741 | 34.258 | 1.00 | 26.08 | C |
| ATOM | 5387 | C | ALA C | 119 | 87.654 | 13.022 | 31.899 | 1.00 | 24.81 | C |
| ATOM | 5388 | O | ALA C | 119 | 87.489 | 11.852 | 31.540 | 1.00 | 23.57 | C |
| ATOM | 5389 | N | GLY C | 120 | 87.282 | 14.060 | 31.154 | 1.00 | 22.99 | C |
| ATOM | 5390 | CA | GLY C | 120 | 86.695 | 13.840 | 29.850 | 1.00 | 23.36 | C |
| ATOM | 5391 | C | GLY C | 120 | 87.654 | 13.095 | 28.932 | 1.00 | 23.32 | C |
| ATOM | 5392 | O | GLY C | 120 | 87.284 | 12.124 | 28.282 | 1.00 | 22.39 | C |
| ATOM | 5393 | N | CYS C | 121 | 88.902 | 13.541 | 28.901 | 1.00 | 23.17 | C |
| ATOM | 5394 | CA | CYS C | 121 | 89.918 | 12.935 | 28.065 | 1.00 | 23.66 | C |
| ATOM | 5395 | CB | CYS C | 121 | 91.184 | 13.762 | 28.144 | 1.00 | 24.22 | C |
| ATOM | 5396 | SG | CYS C | 121 | 90.981 | 15.370 | 27.390 | 1.00 | 31.59 | C |
| ATOM | 5397 | C | CYS C | 121 | 90.215 | 11.497 | 28.425 | 1.00 | 24.08 | C |
| ATOM | 5398 | O | CYS C | 121 | 90.601 | 10.703 | 27.570 | 1.00 | 25.40 | C |
| ATOM | 5399 | N | LYS C | 122 | 90.026 | 11.155 | 29.692 | 1.00 | 25.04 | C |
| ATOM | 5400 | CA | LYS C | 122 | 90.270 | 9.796 | 30.138 | 1.00 | 24.59 | C |
| ATOM | 5401 | CB | LYS C | 122 | 90.293 | 9.740 | 31.672 | 1.00 | 25.68 | C |
| ATOM | 5402 | CG | LYS C | 122 | 91.396 | 10.599 | 32.270 | 1.00 | 26.14 | C |
| ATOM | 5403 | CD | LYS C | 122 | 91.580 | 10.400 | 33.763 | 1.00 | 27.91 | C |
| ATOM | 5404 | CE | LYS C | 122 | 92.594 | 11.421 | 34.315 | 1.00 | 29.43 | C |
| ATOM | 5405 | NZ | LYS C | 122 | 93.121 | 11.095 | 35.679 | 1.00 | 31.16 | C |
| ATOM | 5406 | C | LYS C | 122 | 89.179 | 8.897 | 29.571 | 1.00 | 24.85 | C |
| ATOM | 5407 | O | LYS C | 122 | 89.451 | 7.798 | 29.080 | 1.00 | 24.38 | C |
| ATOM | 5408 | N | VAL C | 123 | 87.940 | 9.366 | 29.611 | 1.00 | 26.05 | C |
| ATOM | 5409 | CA | VAL C | 123 | 86.856 | 8.555 | 29.071 | 1.00 | 27.70 | C |
| ATOM | 5410 | CE | VAL C | 123 | 85.509 | 9.274 | 29.197 | 1.00 | 28.31 | C |
| ATOM | 5411 | CG1 | VAL C | 123 | 84.389 | 8.383 | 28.667 | 1.00 | 28.23 | C |
| ATOM | 5412 | CG2 | VAL C | 123 | 85.272 | 9.627 | 30.650 | 1.00 | 26.87 | C |
| ATOM | 5413 | C | VAL C | 123 | 87.142 | 8.223 | 27.606 | 1.00 | 27.72 | C |
| ATOM | 5414 | O | VAL C | 123 | 86.946 | 7.083 | 27.165 | 1.00 | 27.83 | C |
| ATOM | 5415 | N | VAL C | 124 | 87.618 | 9.212 | 26.855 | 1.00 | 27.26 | C |
| ATOM | 5416 | CA | VAL C | 124 | 87.947 | 8.982 | 25.452 | 1.00 | 27.88 | C |
| ATOM | 5417 | CB | VAL C | 124 | 88.458 | 10.274 | 24.776 | 1.00 | 26.84 | C |
| ATOM | 5418 | CG1 | VAL C | 124 | 88.953 | 9.959 | 23.368 | 1.00 | 26.47 | C |
| ATOM | 5419 | CG2 | VAL C | 124 | 87.322 | 11.304 | 24.713 | 1.00 | 23.58 | C |
| ATOM | 5420 | C | VAL C | 124 | 89.008 | 7.880 | 25.343 | 1.00 | 28.21 | C |
| ATOM | 5421 | O | VAL C | 124 | 88.830 | 6.898 | 24.599 | 1.00 | 27.09 | C |
| ATOM | 5422 | N | ALA C | 125 | 90.097 | 8.041 | 26.095 | 1.00 | 26.95 | C |
| ATOM | 5423 | CA | ALA C | 125 | 91.170 | 7.050 | 26.102 | 1.00 | 29.33 | C |
| ATOM | 5424 | CB | ALA C | 125 | 92.268 | 7.479 | 27.044 | 1.00 | 27.76 | C |
| ATOM | 5425 | C | ALA C | 125 | 90.653 | 5.661 | 26.501 | 1.00 | 31.95 | C |
| ATOM | 5426 | O | ALA C | 125 | 91.063 | 4.647 | 25.930 | 1.00 | 32.25 | C |
| ATOM | 5427 | N | GLU C | 126 | 89.749 | 5.617 | 27.476 | 1.00 | 34.71 | C |
| ATOM | 5428 | CA | CLU C | 126 | 89.179 | 4.356 | 27.919 | 1.00 | 36.70 | C |
| ATOM | 5429 | CE | GLU C | 126 | 88.333 | 4.572 | 29.173 | 1.00 | 39.48 | C |
| ATOM | 5430 | CG | GLU C | 126 | 87.803 | 3.290 | 29.782 | 1.00 | 45.29 | C |
| ATOM | 5431 | CD | GLU C | 126 | 88.919 | 2.320 | 30.166 | 1.00 | 50.81 | C |
| ATOM | 5432 | OE1 | GLU C | 126 | 89.934 | 2.813 | 30.740 | 1.00 | 52.04 | C |
| ATOM | 5433 | OE2 | GLU C | 126 | 88.780 | 1.083 | 29.904 | 1.00 | 50.45 | C |
| ATOM | 5434 | C | GLU C | 126 | 88.325 | 3.739 | 26.813 | 1.00 | 35.98 | C |
| ATOM | 5435 | O | GLU C | 126 | 87.849 | 2.628 | 26.968 | 1.00 | 33.64 | C |
| ATOM | 5436 | N | MSE C | 127 | 88.123 | 4.477 | 25.717 | 1.00 | 37.74 | C |
| ATOM | 5437 | CA | MSE C | 127 | 87.355 | 3.994 | 24.563 | 1.00 | 39.73 | C |
| ATOM | 5438 | CB | MSE C | 127 | 86.592 | 5.129 | 23.866 | 1.00 | 41.63 | C |
| ATOM | 5439 | CG | MSE C | 127 | 85.526 | 5.848 | 24.700 | 1.00 | 42.19 | C |
| ATOM | 5440 | SE | MSE C | 127 | 84.412 | 7.110 | 23.629 | 1.00 | 43.57 | C |
| ATOM | 5441 | CE | MSE C | 127 | 82.969 | 5.916 | 23.133 | 1.00 | 43.96 | C |
| ATOM | 5442 | C | MSE C | 127 | 88.295 | 3.377 | 23.530 | 1.00 | 40.34 | C |
| ATOM | 5443 | O | MSE C | 127 | 87.885 | 2.517 | 22.753 | 1.00 | 41.30 | C |
| ATOM | 5444 | N | ILE C | 128 | 89.544 | 3.835 | 23.506 | 1.00 | 40.42 | C |
| ATOM | 5445 | CA | ILE C | 128 | 90.516 | 3.309 | 22.559 | 1.00 | 39.85 | C |
| ATOM | 5446 | CE | ILE C | 128 | 91.552 | 4.378 | 22.130 | 1.00 | 39.70 | C |
| ATOM | 5447 | CG2 | ILE C | 128 | 92.689 | 3.746 | 21.349 | 1.00 | 39.51 | C |
| ATOM | 5448 | CG1 | ILE C | 128 | 90.879 | 5.426 | 21.254 | 1.00 | 38.45 | C |
| ATOM | 5449 | CD1 | ILE C | 128 | 90.118 | 6.401 | 22.035 | 1.00 | 40.80 | C |
| ATOM | 5450 | C | ILE C | 128 | 91.245 | 2.174 | 23.224 | 1.00 | 39.58 | C |
| ATOM | 5451 | O | ILE C | 128 | 91.534 | 1.156 | 22.603 | 1.00 | 40.22 | C |
| ATOM | 5452 | N | ARG C | 129 | 91.518 | 2.353 | 24.508 | 1.00 | 39.78 | C |
| ATOM | 5453 | CA | ARG C | 129 | 92.230 | 1.362 | 25.300 | 1.00 | 38.47 | C |
| ATOM | 5454 | CB | ARG C | 129 | 91.959 | 1.599 | 26.781 | 1.00 | 39.40 | C |
| ATOM | 5455 | CG | ARG C | 129 | 93.052 | 1.083 | 27.689 | 1.00 | 38.81 | C |
| ATOM | 5456 | CD | ARG C | 129 | 92.731 | 1.379 | 29.130 | 1.00 | 40.09 | C |
| ATOM | 5457 | NE | ARG C | 129 | 92.640 | 2.812 | 29.456 | 1.00 | 42.40 | C |
| ATOM | 5458 | CZ | ARG C | 129 | 93.651 | 3.680 | 29.437 | 1.00 | 41.54 | C |
| ATOM | 5459 | NH1 | ARG C | 129 | 94.869 | 3.283 | 29.089 | 1.00 | 41.83 | C |
| ATOM | 5460 | NH2 | ARG C | 129 | 93.443 | 4.937 | 29.815 | 1.00 | 39.32 | C |
| ATOM | 5461 | C | ARG C | 129 | 91.863 | −0.066 | 24.940 | 1.00 | 37.14 | C |
| ATOM | 5462 | O | ARG C | 129 | 90.682 | −0.428 | 24.875 | 1.00 | 34.49 | C |
| ATOM | 5463 | N | CLY C | 130 | 92.900 | −0.860 | 24.688 | 1.00 | 36.58 | C |
| ATOM | 5464 | CA | GLY C | 130 | 92.732 | −2.264 | 24.356 | 1.00 | 36.84 | C |
| ATOM | 5465 | C | GLY C | 130 | 92.098 | −2.656 | 23.032 | 1.00 | 36.85 | C |
| ATOM | 5466 | O | GLY C | 130 | 92.210 | −3.816 | 22.626 | 1.00 | 37.50 | C |
| ATOM | 5467 | N | ARG C | 131 | 91.431 | −1.721 | 22.359 | 1.00 | 37.11 | C |
| ATOM | 5468 | CA | ARG C | 131 | 90.787 | −2.017 | 21.073 | 1.00 | 37.10 | C |
| ATOM | 5469 | CB | ARG C | 131 | 89.711 | −0.973 | 20.771 | 1.00 | 39.22 | C |
| ATOM | 5470 | CG | ARG C | 131 | 88.431 | −1.131 | 21.569 | 1.00 | 41.84 | C |
| ATOM | 5471 | CD | ARG C | 131 | 87.839 | −2.509 | 21.363 | 1.00 | 45.09 | C |
| ATOM | 5472 | NE | ARG C | 131 | 86.396 | −2.558 | 21.604 | 1.00 | 47.90 | C |
| ATOM | 5473 | CZ | ARG C | 131 | 85.479 | −2.055 | 20.780 | 1.00 | 49.21 | C |
| ATOM | 5474 | NH1 | ARG C | 131 | 85.846 | −1.459 | 19.656 | 1.00 | 49.53 | C |
| ATOM | 5475 | NH2 | ARG C | 131 | 84.191 | −2.155 | 21.073 | 1.00 | 51.64 | C |
| ATOM | 5476 | C | ARG C | 131 | 91.760 | −2.084 | 19.894 | 1.00 | 35.38 | C |
| ATOM | 5477 | O | ARG C | 131 | 92.917 | −1.676 | 19.989 | 1.00 | 34.16 | C |
| ATOM | 5478 | N | SER C | 132 | 91.277 | −2.610 | 18.781 | 1.00 | 33.40 | C |
| ATOM | 5479 | CA | SER C | 132 | 92.093 | −2.717 | 17.585 | 1.00 | 32.90 | C |
| ATOM | 5480 | CB | SER C | 132 | 91.933 | −4.096 | 16.966 | 1.00 | 31.03 | C |
| ATOM | 5481 | OC | SER C | 132 | 90.581 | −4.307 | 16.607 | 1.00 | 31.69 | C |
| ATOM | 5482 | C | SER C | 132 | 91.646 | −1.661 | 16.576 | 1.00 | 32.11 | C |
| ATOM | 5483 | O | SER C | 132 | 90.585 | −1.054 | 16.738 | 1.00 | 30.91 | C |
| ATOM | 5484 | N | PRO C | 133 | 92.462 | −1.422 | 15.532 | 1.00 | 31.16 | C |
| ATOM | 5485 | CD | PRO C | 133 | 93.848 | −1.900 | 15.407 | 1.00 | 29.35 | C |
| ATOM | 5486 | CA | PRO C | 133 | 92.180 | −0.451 | 14.480 | 1.00 | 32.65 | C |
| ATOM | 5487 | CB | PRO C | 133 | 93.219 | −0.801 | 13.441 | 1.00 | 29.52 | C |
| ATOM | 5488 | CG | PRO C | 133 | 94.400 | −1.021 | 14.307 | 1.00 | 29.23 | C |
| ATOM | 5489 | C | PRO C | 133 | 90.754 | −0.514 | 13.963 | 1.00 | 35.83 | C |
| ATOM | 5490 | O | PRO C | 133 | 90.054 | 0.504 | 13.909 | 1.00 | 36.35 | C |
| ATOM | 5491 | N | GLU C | 134 | 90.318 | −1.709 | 13.588 | 1.00 | 38.45 | C |
| ATOM | 5492 | CA | GLU C | 134 | 88.962 | −1.875 | 13.104 | 1.00 | 41.11 | C |
| ATOM | 5493 | CB | GLU C | 134 | 88.765 | −3.265 | 12.494 | 1.00 | 44.05 | C |
| ATOM | 5494 | CG | GLU C | 134 | 88.697 | −3.264 | 10.965 | 1.00 | 49.94 | C |
| ATOM | 5495 | CD | GLU C | 134 | 87.779 | −2.177 | 10.405 | 1.00 | 52.39 | C |
| ATOM | 5496 | OE1 | GLU C | 134 | 86.664 | −2.011 | 10.958 | 1.00 | 54.43 | C |
| ATOM | 5497 | OE2 | GLU C | 134 | 88.173 | −1.503 | 9.413 | 1.00 | 51.18 | C |
| ATOM | 5498 | C | GLU C | 134 | 87.968 | −1.660 | 14.249 | 1.00 | 41.19 | C |
| ATOM | 5499 | O | GLU C | 134 | 86.913 | −1.040 | 14.062 | 1.00 | 40.64 | C |
| ATOM | 5500 | N | GLU C | 135 | 88.297 | −2.162 | 15.437 | 1.00 | 41.33 | C |
| ATOM | 5501 | CA | GLU C | 135 | 87.399 | −1.978 | 16.575 | 1.00 | 42.01 | C |
| ATOM | 5502 | CB | GLU C | 135 | 87.915 | −2.697 | 17.820 | 1.00 | 42.98 | C |
| ATOM | 5503 | CG | GLU C | 135 | 87.961 | −4.198 | 17.745 | 1.00 | 44.16 | C |
| ATOM | 5504 | CD | GLU C | 135 | 88.393 | −4.796 | 19.073 | 1.00 | 46.95 | C |
| ATOM | 5505 | OE1 | GLU C | 135 | 89.419 | −4.323 | 19.629 | 1.00 | 46.77 | C |
| ATOM | 5506 | OE2 | GLU C | 135 | 87.712 | −5.728 | 19.565 | 1.00 | 48.28 | C |
| ATOM | 5507 | C | GLU C | 135 | 87.226 | −0.489 | 16.908 | 1.00 | 40.82 | C |
| ATOM | 5508 | O | GLU C | 135 | 86.152 | −0.055 | 17.328 | 1.00 | 42.69 | C |
| ATOM | 5509 | N | ILE C | 136 | 88.284 | 0.292 | 16.718 | 1.00 | 37.69 | C |
| ATOM | 5510 | CA | ILE C | 136 | 88.224 | 1.718 | 16.997 | 1.00 | 33.59 | C |
| ATOM | 5511 | CB | ILE C | 136 | 89.638 | 2.316 | 17.096 | 1.00 | 32.05 | C |
| ATOM | 5512 | CG2 | ILE C | 136 | 89.552 | 3.838 | 17.349 | 1.00 | 27.02 | C |
| ATOM | 5513 | CG1 | ILE C | 136 | 90.412 | 1.564 | 18.196 | 1.00 | 29.01 | C |
| ATOM | 5514 | CD1 | ILE C | 136 | 91.859 | 1.992 | 18.390 | 1.00 | 26.23 | C |
| ATOM | 5515 | C | ILE C | 136 | 87.442 | 2.419 | 15.903 | 1.00 | 33.18 | C |
| ATOM | 5516 | O | ILE C | 136 | 86.631 | 3.299 | 16.171 | 1.00 | 31.83 | C |
| ATOM | 5517 | N | ARG C | 137 | 87.669 | 2.021 | 14.661 | 1.00 | 34.42 | C |
| ATOM | 5518 | CA | ARG C | 137 | 86.955 | 2.652 | 13.566 | 1.00 | 34.64 | C |
| ATOM | 5519 | CB | ARG C | 137 | 87.352 | 2.008 | 12.253 | 1.00 | 33.73 | C |
| ATOM | 5520 | CG | ARG C | 137 | 88.809 | 2.218 | 11.916 | 1.00 | 34.55 | C |
| ATOM | 5521 | CD | ARG C | 137 | 89.226 | 1.238 | 10.851 | 1.00 | 34.62 | C |
| ATOM | 5522 | NE | ARG C | 137 | 90.665 | 1.216 | 10.644 | 1.00 | 33.99 | C |
| ATOM | 5523 | CZ | ARG C | 137 | 91.340 | 0.120 | 10.328 | 1.00 | 34.12 | C |
| ATOM | 5524 | NH1 | ARG C | 137 | 90.694 | −1.029 | 10.197 | 1.00 | 32.84 | C |
| ATOM | 5525 | NH2 | ARG C | 137 | 92.651 | 0.176 | 10.125 | 1.00 | 34.77 | C |
| ATOM | 5526 | C | ARG C | 137 | 85.454 | 2.549 | 13.774 | 1.00 | 35.03 | C |
| ATOM | 5527 | O | ARG C | 137 | 84.745 | 3.538 | 13.638 | 1.00 | 34.65 | C |
| ATOM | 5528 | N | ARG C | 138 | 84.968 | 1.366 | 14.132 | 1.00 | 35.13 | C |
| ATOM | 5529 | CA | ARG C | 138 | 83.539 | 1.211 | 14.328 | 1.00 | 36.00 | C |
| ATOM | 5530 | CB | ARG C | 138 | 83.176 | −0.270 | 14.439 | 1.00 | 38.78 | C |
| ATOM | 5531 | CG | ARG C | 138 | 83.339 | −1.037 | 13.114 | 1.00 | 43.46 | C |
| ATOM | 5532 | CD | ARG C | 138 | 82.329 | −0.608 | 12.030 | 0.00 | 47.14 | C |
| ATOM | 5533 | NE | ARG C | 138 | 82.537 | 0.734 | 11.466 | 0.00 | 51.00 | C |
| ATOM | 5534 | CZ | ARG C | 138 | 83.462 | 1.061 | 10.563 | 1.00 | 54.05 | C |
| ATOM | 5535 | NH1 | ARG C | 138 | 84.302 | 0.146 | 10.095 | 1.00 | 55.91 | C |
| ATOM | 5536 | NH2 | ARG C | 138 | 83.532 | 2.307 | 10.105 | 1.00 | 54.20 | C |
| ATOM | 5537 | C | ARG C | 138 | 83.008 | 1.990 | 15.528 | 1.00 | 34.39 | C |
| ATOM | 5538 | O | ARG C | 138 | 81.816 | 2.264 | 15.626 | 1.00 | 34.40 | C |
| ATOM | 5539 | N | THR C | 139 | 83.895 | 2.377 | 16.426 | 1.00 | 31.97 | C |
| ATOM | 5540 | CA | THR C | 139 | 83.486 | 3.123 | 17.596 | 1.00 | 29.98 | C |
| ATOM | 5541 | CB | THR C | 139 | 84.575 | 3.077 | 18.624 | 1.00 | 30.24 | C |
| ATOM | 5542 | OG1 | THR C | 139 | 84.946 | 1.713 | 18.825 | 1.00 | 31.86 | C |
| ATOM | 5543 | CG2 | THR C | 139 | 84.120 | 3.697 | 19.923 | 1.00 | 28.26 | C |
| ATOM | 5544 | C | THR C | 139 | 83.214 | 4.573 | 17.265 | 1.00 | 29.86 | C |
| ATOM | 5545 | O | THR C | 139 | 82.267 | 5.180 | 17.767 | 1.00 | 30.37 | C |
| ATOM | 5546 | N | PHE C | 140 | 84.053 | 5.138 | 16.415 | 1.00 | 29.19 | C |
| ATOM | 5547 | CA | PHE C | 140 | 83.883 | 6.525 | 16.045 | 1.00 | 28.27 | C |
| ATOM | 5548 | CB | PHE C | 140 | 85.229 | 7.230 | 16.152 | 1.00 | 25.82 | C |
| ATOM | 5549 | CG | PHE C | 140 | 85.797 | 7.223 | 17.537 | 1.00 | 23.52 | C |
| ATOM | 5550 | CD1 | PHE C | 140 | 85.228 | 7.990 | 18.543 | 1.00 | 22.27 | C |
| ATOM | 5551 | CD2 | PHE C | 140 | 86.907 | 6.452 | 17.844 | 1.00 | 22.34 | C |
| ATOM | 5552 | CE1 | PHE C | 140 | 85.767 | 7.982 | 19.839 | 1.00 | 20.23 | C |
| ATOM | 5553 | CE2 | PHE C | 140 | 87.450 | 6.441 | 19.135 | 1.00 | 18.70 | C |
| ATOM | 5554 | CZ | PHE C | 140 | 86.880 | 7.204 | 20.126 | 1.00 | 17.04 | C |
| ATOM | 5555 | C | PHE C | 140 | 83.307 | 6.697 | 14.647 | 1.00 | 28.75 | C |
| ATOM | 5556 | O | PHE C | 140 | 83.087 | 7.822 | 14.221 | 1.00 | 30.09 | C |
| ATOM | 5557 | N | ASN C | 141 | 83.027 | 5.585 | 13.964 | 1.00 | 29.40 | C |
| ATOM | 5558 | CA | ASN C | 141 | 82.521 | 5.589 | 12.580 | 1.00 | 30.10 | C |
| ATOM | 5559 | CB | ASN C | 141 | 81.182 | 6.335 | 12.434 | 1.00 | 30.86 | C |
| ATOM | 5560 | CG | ASN C | 141 | 80.620 | 6.279 | 10.991 | 1.00 | 32.35 | C |
| ATOM | 5561 | OD1 | ASN C | 141 | 80.549 | 5.206 | 10.385 | 1.00 | 30.84 | C |
| ATOM | 5562 | ND2 | ASN C | 141 | 80.212 | 7.436 | 10.453 | 1.00 | 32.26 | C |
| ATOM | 5563 | C | ASN C | 141 | 83.554 | 6.256 | 11.680 | 1.00 | 30.32 | C |
| ATOM | 5564 | O | ASN C | 141 | 83.223 | 7.047 | 10.803 | 1.00 | 31.59 | C |
| ATOM | 5565 | N | ILE C | 142 | 84.821 | 5.953 | 11.907 | 1.00 | 29.73 | C |
| ATOM | 5566 | CA | ILE C | 142 | 85.867 | 6.548 | 11.091 | 1.00 | 30.11 | C |
| ATOM | 5567 | CB | ILE C | 142 | 87.151 | 6.830 | 11.983 | 1.00 | 29.75 | C |
| ATOM | 5568 | CG2 | ILE C | 142 | 87.180 | 5.925 | 13.180 | 1.00 | 31.60 | C |
| ATOM | 5569 | CG1 | ILE C | 142 | 88.428 | 6.712 | 11.164 | 1.00 | 29.84 | C |
| ATOM | 5570 | CD1 | ILE C | 142 | 88.925 | 8.056 | 10.663 | 1.00 | 36.02 | C |
| ATOM | 5571 | C | ILE C | 142 | 86.129 | 5.630 | 9.885 | 1.00 | 28.78 | C |
| ATOM | 5572 | O | ILE C | 142 | 86.070 | 4.412 | 10.004 | 1.00 | 27.70 | C |
| ATOM | 5573 | N | VAL C | 143 | 86.355 | 6.201 | 8.708 | 1.00 | 28.81 | C |
| ATOM | 5574 | CA | VAL C | 143 | 86.594 | 5.344 | 7.556 | 1.00 | 28.92 | C |
| ATOM | 5575 | CB | VAL C | 143 | 86.022 | 5.937 | 6.250 | 1.00 | 27.33 | C |
| ATOM | 5576 | CG1 | VAL C | 143 | 86.549 | 5.165 | 5.056 | 1.00 | 27.92 | C |
| ATOM | 5577 | CG2 | VAL C | 143 | 84.494 | 5.866 | 6.269 | 1.00 | 26.26 | C |
| ATOM | 5578 | C | VAL C | 143 | 88.071 | 5.084 | 7.356 | 1.00 | 28.95 | C |
| ATOM | 5579 | O | VAL C | 143 | 88.905 | 5.971 | 7.539 | 1.00 | 31.52 | C |
| ATOM | 5580 | N | ASN C | 144 | 88.387 | 3.845 | 7.006 | 1.00 | 27.50 | C |
| ATOM | 5581 | CA | ASN C | 144 | 89.754 | 3.434 | 6.761 | 1.00 | 26.06 | C |
| ATOM | 5582 | CB | ASN C | 144 | 89.837 | 1.907 | 6.798 | 1.00 | 27.42 | C |
| ATOM | 5583 | CG | ASN C | 144 | 91.258 | 1.377 | 6.661 | 1.00 | 28.39 | C |
| ATOM | 5584 | OD1 | ASN C | 144 | 91.528 | 0.242 | 7.035 | 1.00 | 28.70 | C |
| ATOM | 5585 | ND2 | ASN C | 144 | 92.162 | 2.185 | 6.119 | 1.00 | 30.75 | C |
| ATOM | 5586 | C | ASN C | 144 | 90.074 | 3.942 | 5.371 | 1.00 | 26.48 | C |
| ATOM | 5587 | O | ASN C | 144 | 89.519 | 3.458 | 4.394 | 1.00 | 26.51 | C |
| ATOM | 5588 | N | ASP C | 145 | 90.964 | 4.918 | 5.277 | 1.00 | 26.34 | C |
| ATOM | 5589 | CA | ASP C | 145 | 91.315 | 5.492 | 3.988 | 1.00 | 27.61 | C |
| ATOM | 5590 | CB | ASP C | 145 | 91.245 | 7.018 | 4.058 | 1.00 | 28.15 | C |
| ATOM | 5591 | CG | ASP C | 145 | 91.840 | 7.570 | 5.336 | 1.00 | 28.20 | C |
| ATOM | 5592 | OD1 | ASP C | 145 | 92.508 | 6.808 | 6.080 | 1.00 | 26.63 | C |
| ATOM | 5593 | OD2 | ASP C | 145 | 91.639 | 8.780 | 5.595 | 1.00 | 29.52 | C |
| ATOM | 5594 | C | ASP C | 145 | 92.684 | 5.086 | 3.479 | 1.00 | 28.92 | C |
| ATOM | 5595 | O | ASP C | 145 | 93.208 | 5.708 | 2.549 | 1.00 | 29.06 | C |
| ATOM | 5596 | N | PHE C | 146 | 93.262 | 4.050 | 4.083 | 1.00 | 28.68 | C |
| ATOM | 5597 | CA | PHE C | 146 | 94.585 | 3.572 | 3.685 | 1.00 | 29.48 | C |
| ATOM | 5598 | CB | PHE C | 146 | 95.222 | 2.744 | 4.799 | 1.00 | 31.05 | C |
| ATOM | 5599 | CG | PHE C | 146 | 95.709 | 3.550 | 5.955 | 1.00 | 33.63 | C |
| ATOM | 5600 | CO1 | PHE C | 146 | 96.641 | 4.574 | 5.761 | 1.00 | 34.53 | C |
| ATOM | 5601 | CD2 | PHE C | 146 | 95.277 | 3.254 | 7.250 | 1.00 | 33.58 | C |
| ATOM | 5602 | CE1 | PHE C | 146 | 97.147 | 5.292 | 6.841 | 1.00 | 35.14 | C |
| ATOM | 5603 | CE2 | PHE C | 146 | 95.767 | 3.956 | 8.338 | 1.00 | 34.74 | C |
| ATOM | 5604 | CZ | PHE C | 146 | 96.709 | 4.981 | 8.136 | 1.00 | 36.19 | C |
| ATOM | 5605 | C | PHE C | 146 | 94.596 | 2.727 | 2.431 | 1.00 | 28.86 | C |
| ATOM | 5606 | O | PHE C | 146 | 93.889 | 1.725 | 2.355 | 1.00 | 30.57 | C |
| ATOM | 5607 | N | THR C | 147 | 95.423 | 3.109 | 1.460 | 1.00 | 28.83 | C |
| ATOM | 5608 | CA | THR C | 147 | 95.536 | 2.339 | 0.222 | 1.00 | 30.01 | C |
| ATOM | 5609 | CB | THR C | 147 | 96.556 | 2.946 | −0.783 | 1.00 | 29.36 | C |
| ATOM | 5610 | OG1 | THR C | 147 | 97.817 | 3.147 | −0.128 | 1.00 | 29.97 | C |
| ATOM | 5611 | CG2 | THR C | 147 | 96.046 | 4.252 | −1.355 | 1.00 | 27.00 | C |
| ATOM | 5612 | C | THR C | 147 | 96.049 | 0.948 | 0.552 | 1.00 | 30.37 | C |
| ATOM | 5613 | O | THR C | 147 | 96.708 | 0.745 | 1.563 | 1.00 | 31.13 | C |
| ATOM | 5614 | N | PRO C | 148 | 95.751 | −0.032 | −0.299 | 1.00 | 31.61 | C |
| ATOM | 5615 | CD | PRO C | 148 | 94.879 | −0.031 | −1.485 | 1.00 | 31.67 | C |
| ATOM | 5616 | CA | PRO C | 148 | 96.241 | −1.375 | −0.010 | 1.00 | 32.60 | C |
| ATOM | 5617 | CB | PRO C | 148 | 95.903 | −2.141 | −1.282 | 1.00 | 31.26 | C |
| ATOM | 5618 | CG | PRO C | 148 | 94.611 | −1.527 | −1.697 | 1.00 | 30.19 | C |
| ATOM | 5619 | C | PRO C | 148 | 97.734 | −1.327 | 0.255 | 1.00 | 34.28 | C |
| ATOM | 5620 | O | PRO C | 148 | 98.244 | −2.067 | 1.087 | 1.00 | 35.43 | C |
| ATOM | 5621 | N | GLU C | 149 | 98.441 | −0.445 | −0.437 | 1.00 | 37.73 | C |
| ATOM | 5622 | CA | GLU C | 149 | 99.873 | −0.372 | −0.221 | 1.00 | 41.17 | C |
| ATOM | 5623 | CB | GLU C | 149 | 100.532 | 0.535 | −1.266 | 1.00 | 39.75 | C |
| ATOM | 5624 | CG | GLU C | 149 | 102.038 | 0.662 | −1.083 | 1.00 | 39.00 | C |
| ATOM | 5625 | CD | GLU C | 149 | 102.703 | 1.461 | −2.171 | 1.00 | 39.73 | C |
| ATOM | 5626 | OE1 | GLU C | 149 | 102.443 | 2.683 | −2.265 | 1.00 | 39.67 | C |
| ATOM | 5627 | OE2 | GLU C | 149 | 103.490 | 0.868 | −2.938 | 1.00 | 39.04 | C |
| ATOM | 5628 | C | GLU C | 149 | 100.183 | 0.120 | 1.195 | 1.00 | 44.52 | C |
| ATOM | 5629 | O | GLU C | 149 | 100.646 | −0.656 | 2.032 | 1.00 | 44.69 | C |
| ATOM | 5630 | N | GUI C | 150 | 99.914 | 1.398 | 1.460 | 1.00 | 48.03 | C |
| ATOM | 5631 | CA | GLU C | 150 | 100.161 | 1.995 | 2.774 | 1.00 | 51.71 | C |
| ATOM | 5632 | CB | GLU C | 150 | 99.557 | 3.407 | 2.822 | 1.00 | 52.60 | C |
| ATOM | 5633 | CG | GLU C | 150 | 100.095 | 4.362 | 1.763 | 1.00 | 56.26 | C |
| ATOM | 5634 | CD | GLU C | 150 | 101.508 | 4.869 | 2.052 | 1.00 | 57.57 | C |
| ATOM | 5635 | OE1 | GLU C | 150 | 101.735 | 5.354 | 3.178 | 1.00 | 58.02 | C |
| ATOM | 5636 | OE2 | GLU C | 150 | 102.381 | 4.803 | 1.153 | 1.00 | 58.12 | C |
| ATOM | 5637 | C | GLU C | 150 | 99.603 | 1.136 | 3.938 | 1.00 | 53.38 | C |
| ATOM | 5638 | O | GLU C | 150 | 100.159 | 1.112 | 5.042 | 1.00 | 52.61 | C |
| ATOM | 5639 | N | GLU C | 151 | 98.515 | 0.420 | 3.677 | 1.00 | 55.17 | C |
| ATOM | 5640 | CA | GLU C | 151 | 97.891 | −0.431 | 4.681 | 1.00 | 58.21 | C |
| ATOM | 5641 | CB | GLU C | 151 | 96.636 | −1.087 | 4.097 | 1.00 | 60.47 | C |
| ATOM | 5642 | CG | GLU C | 151 | 95.349 | −0.835 | 4.868 | 1.00 | 62.11 | C |
| ATOM | 5643 | CD | GLU C | 151 | 94.154 | −1.512 | 4.222 | 1.00 | 63.63 | C |
| ATOM | 5644 | OE1 | GLU C | 151 | 94.025 | −1.410 | 2.978 | 1.00 | 64.05 | C |
| ATOM | 5645 | OE2 | GLU C | 151 | 93.347 | −2.135 | 4.955 | 1.00 | 63.74 | C |
| ATOM | 5646 | C | GLU C | 151 | 98.845 | −1.519 | 5.182 | 1.00 | 59.56 | C |
| ATOM | 5647 | O | GLU C | 151 | 99.118 | −1.609 | 6.375 | 1.00 | 60.86 | C |
| ATOM | 5648 | N | ALA C | 152 | 99.345 | −2.346 | 4.269 | 1.00 | 60.60 | C |
| ATOM | 5649 | CA | ALA C | 152 | 100.263 | −3.422 | 4.626 | 1.00 | 61.88 | C |
| ATOM | 5650 | CB | ALA C | 152 | 100.455 | −4.356 | 3.439 | 1.00 | 61.89 | C |
| ATOM | 5651 | C | ALA C | 152 | 101.616 | −2.881 | 5.080 | 1.00 | 62.64 | C |
| ATOM | 5652 | O | ALA C | 152 | 102.398 | −3.589 | 5.699 | 1.00 | 62.91 | C |
| ATOM | 5653 | N | ALA C | 153 | 101.888 | −1.622 | 4.769 | 1.00 | 64.35 | C |
| ATOM | 5654 | CA | ALA C | 153 | 103.146 | −1.005 | 5.144 | 1.00 | 66.65 | C |
| ATOM | 5655 | CB | ALA C | 153 | 103.441 | 0.166 | 4.222 | 1.00 | 66.57 | C |
| ATOM | 5656 | C | ALA C | 153 | 103.114 | −0.534 | 6.593 | 1.00 | 68.35 | C |
| ATOM | 5657 | O | ALA C | 153 | 104.159 | −0.238 | 7.177 | 1.00 | 67.40 | C |
| ATOM | 5658 | N | ILE C | 154 | 101.913 | −0.463 | 7.167 | 1.00 | 70.84 | C |
| ATOM | 5659 | CA | ILE C | 154 | 101.753 | −0.021 | 8.549 | 1.00 | 73.04 | C |
| ATOM | 5660 | CB | ILE C | 154 | 100.276 | 0.333 | 8.874 | 1.00 | 72.44 | C |
| ATOM | 5661 | CG2 | ILE C | 154 | 100.160 | 0.823 | 10.308 | 0.00 | 73.15 | C |
| ATOM | 5662 | CG1 | ILE C | 154 | 99.768 | 1.405 | 7.906 | 0.00 | 73.18 | C |
| ATOM | 5663 | CD1 | ILE C | 154 | 100.596 | 2.681 | 7.891 | 0.00 | 73.32 | C |
| ATOM | 5664 | C | ILE C | 154 | 102.242 | −1.083 | 9.530 | 1.00 | 74.39 | C |
| ATOM | 5665 | O | ILE C | 154 | 103.164 | −0.836 | 10.314 | 1.00 | 75.10 | C |
| ATOM | 5666 | N | ARG C | 155 | 101.638 | −2.267 | 9.481 | 1.00 | 75.33 | C |
| ATOM | 5667 | CA | ARG C | 155 | 102.035 | −3.343 | 10.386 | 1.00 | 77.14 | C |
| ATOM | 5668 | CB | ARG C | 155 | 101.354 | −4.655 | 9.970 | 1.00 | 77.56 | C |
| ATOM | 5669 | CG | ARG C | 155 | 101.473 | −4.969 | 8.491 | 1.00 | 79.06 | C |
| ATOM | 5670 | CD | ARG C | 155 | 100.453 | −6.012 | 8.042 | 1.00 | 79.84 | C |
| ATOM | 5671 | NE | ARG C | 155 | 101.032 | −7.346 | 7.880 | 1.00 | 82.03 | C |
| ATOM | 5672 | CZ | ARG C | 155 | 101.458 | −8.120 | 8.876 | 1.00 | 83.21 | C |
| ATOM | 5673 | NH1 | ARG C | 155 | 101.376 | −7.702 | 10.130 | 1.00 | 83.46 | C |
| ATOM | 5674 | NH2 | ARG C | 155 | 101.967 | −9.320 | 8.617 | 1.00 | 83.34 | C |
| ATOM | 5675 | C | ARG C | 155 | 103.557 | −3.521 | 10.429 | 1.00 | 77.49 | C |
| ATOM | 5676 | O | ARG C | 155 | 104.141 | −3.689 | 11.505 | 1.00 | 76.81 | C |
| ATOM | 5677 | N | ARG C | 156 | 104.185 | −3.454 | 9.256 | 1.00 | 78.72 | C |
| ATOM | 5678 | CA | ARG C | 156 | 105.631 | −3.611 | 9.120 | 1.00 | 80.26 | C |
| ATOM | 5679 | CB | ARG C | 156 | 106.371 | −2.543 | 9.936 | 1.00 | 79.66 | C |
| ATOM | 5680 | CG | ARG C | 156 | 106.197 | −1.128 | 9.442 | 1.00 | 78.84 | C |
| ATOM | 5681 | CD | ARG C | 156 | 106.951 | −0.151 | 10.332 | 1.00 | 78.63 | C |
| ATOM | 5682 | NE | ARG C | 156 | 106.958 | 1.201 | 9.777 | 1.00 | 79.37 | C |
| ATOM | 5683 | CZ | ARG C | 156 | 107.560 | 2.243 | 10.344 | 1.00 | 79.78 | C |
| ATOM | 5684 | NH1 | ARG C | 156 | 108.210 | 2.098 | 11.492 | 1.00 | 80.21 | C |
| ATOM | 5685 | NH2 | ARG C | 156 | 107.513 | 3.434 | 9.763 | 1.00 | 79.89 | C |
| ATOM | 5686 | C | ARG C | 156 | 106.075 | −4.997 | 9.586 | 1.00 | 81.59 | C |
| ATOM | 5687 | O | ARG C | 156 | 106.298 | −5.862 | 8.715 | 0.00 | 81.82 | C |
| ATOM | 5688 | OXT | ARG C 156 | 106.182 | −5.207 | 10.817 | 1.00 | 83.28 | C | |
| ATOM | 5689 | CB | LEU D | 270 | 75.377 | 10.305 | 15.206 | 1.00 | 63.79 | D |
| ATOM | 5690 | CG | LEU D | 270 | 75.038 | 9.447 | 13.983 | 1.00 | 64.88 | D |
| ATOM | 5691 | CD1 | LEU D | 270 | 73.550 | 9.589 | 13.707 | 1.00 | 64.89 | D |
| ATOM | 5692 | CD2 | LEU D | 270 | 75.842 | 9.894 | 12.762 | 1.00 | 64.87 | D |
| ATOM | 5693 | C | LEU D | 270 | 77.526 | 9.458 | 16.216 | 1.00 | 61.89 | D |
| ATOM | 5694 | O | LEU D | 270 | 78.274 | 9.273 | 17.176 | 1.00 | 61.69 | D |
| ATOM | 5695 | N | LEU D | 270 | 75.776 | 10.456 | 17.643 | 1.00 | 62.12 | D |
| ATOM | 5696 | CA | LEU D | 270 | 76.015 | 9.627 | 16.425 | 1.00 | 62.72 | D |
| ATOM | 5697 | N | LYS D | 271 | 77.975 | 9.523 | 14.966 | 1.00 | 60.94 | D |
| ATOM | 5698 | CA | LYS D | 271 | 79.394 | 9.377 | 14.666 | 1.00 | 59.23 | D |
| ATOM | 5699 | CB | LYS D | 271 | 79.635 | 9.177 | 13.157 | 1.00 | 61.06 | D |
| ATOM | 5700 | CG | LYS D | 271 | 79.243 | 10.362 | 12.273 | 1.00 | 61.84 | D |
| ATOM | 5701 | CD | LYS D | 271 | 79.810 | 10.233 | 10.859 | 1.00 | 62.36 | D |
| ATOM | 5702 | CE | LYS D | 271 | 81.303 | 10.589 | 10.782 | 1.00 | 63.95 | D |
| ATOM | 5703 | NZ | LYS D | 271 | 82.195 | 9.772 | 11.664 | 1.00 | 62.34 | D |
| ATOM | 5704 | C | LYS D | 271 | 80.146 | 10.607 | 15.114 | 1.00 | 56.61 | D |
| ATOM | 5705 | O | LYS D | 271 | 79.604 | 11.715 | 15.119 | 1.00 | 56.97 | D |
| ATOM | 5706 | N | ARG D | 272 | 81.403 | 10.412 | 15.483 | 1.00 | 53.78 | D |
| ATOM | 5707 | CA | ARG D | 272 | 82.212 | 11.531 | 15.902 | 1.00 | 51.25 | D |
| ATOM | 5708 | CB | ARG D | 272 | 81.625 | 12.153 | 17.159 | 1.00 | 50.62 | D |
| ATOM | 5709 | CG | ARG D | 272 | 81.295 | 13.617 | 16.977 | 1.00 | 50.35 | D |
| ATOM | 5710 | CD | ARG D | 272 | 82.497 | 14.465 | 17.304 | 1.00 | 50.29 | D |
| ATOM | 5711 | NE | ARG D | 272 | 82.835 | 15.461 | 16.290 | 1.00 | 50.91 | D |
| ATOM | 5712 | CZ | ARG D | 272 | 82.084 | 16.507 | 15.950 | 1.00 | 50.34 | D |
| ATOM | 5713 | NH1 | ARG D | 272 | 80.913 | 16.720 | 16.528 | 1.00 | 50.37 | D |
| ATOM | 5714 | NH2 | ARG D | 272 | 82.528 | 17.376 | 15.049 | 1.00 | 50.67 | D |
| ATOM | 5715 | C | ARG D | 272 | 83.652 | 11.142 | 16.124 | 1.00 | 49.27 | D |
| ATOM | 5716 | O | ARG D | 272 | 83.995 | 10.497 | 17.112 | 1.00 | 50.77 | D |
| ATOM | 5717 | N | ASP D | 273 | 84.498 | 11.523 | 15.180 | 1.00 | 45.44 | D |
| ATOM | 5718 | CA | ASP D | 273 | 85.904 | 11.229 | 15.310 | 1.00 | 40.81 | D |
| ATOM | 5719 | CB | ASP D | 273 | 86.582 | 11.281 | 13.951 | 1.00 | 40.44 | D |
| ATOM | 5720 | CG | ASP D | 273 | 87.998 | 10.765 | 13.997 | 1.00 | 40.32 | D |
| ATOM | 5721 | OD1 | ASP D | 273 | 88.731 | 11.138 | 14.939 | 1.00 | 40.48 | D |
| ATOM | 5722 | OD2 | ASP D | 273 | 88.377 | 9.991 | 13.094 | 1.00 | 40.70 | D |
| ATOM | 5723 | C | ASP D | 273 | 86.486 | 12.295 | 16.231 | 1.00 | 38.02 | D |
| ATOM | 5724 | O | ASP D | 273 | 87.082 | 13.269 | 15.771 | 1.00 | 36.97 | D |
| ATOM | 5725 | N | LEU D | 274 | 86.301 | 12.096 | 17.536 | 1.00 | 35.35 | D |
| ATOM | 5726 | CA | LEU D | 274 | 86.783 | 13.026 | 18.562 | 1.00 | 32.44 | D |
| ATOM | 5727 | CB | LEU D | 274 | 86.596 | 12.405 | 19.957 | 1.00 | 32.29 | D |
| ATOM | 5728 | CG | LEU D | 274 | 85.237 | 11.729 | 20.146 | 1.00 | 33.44 | D |
| ATOM | 5729 | CD1 | LEU D | 274 | 85.148 | 10.949 | 21.454 | 1.00 | 33.06 | D |
| ATOM | 5730 | CD2 | LEU D | 274 | 84.185 | 12.796 | 20.076 | 1.00 | 33.99 | D |
| ATOM | 5731 | C | LEU D | 274 | 88.250 | 13.443 | 18.376 | 1.00 | 30.29 | D |
| ATOM | 5732 | O | LEU D | 274 | 88.589 | 14.620 | 18.487 | 1.00 | 29.35 | D |
| ATOM | 5733 | N | ILE D | 275 | 89.119 | 12.481 | 18.090 | 1.00 | 27.97 | D |
| ATOM | 5734 | CA | ILE D | 275 | 90.529 | 12.790 | 17.919 | 1.00 | 26.53 | D |
| ATOM | 5735 | CB | ILE D | 275 | 91.366 | 11.487 | 17.721 | 1.00 | 26.41 | D |
| ATOM | 5736 | CG2 | ILE D | 275 | 92.798 | 11.829 | 17.316 | 1.00 | 25.43 | D |
| ATOM | 5737 | CG1 | ILE D | 275 | 91.390 | 10.679 | 19.029 | 1.00 | 24.46 | D |
| ATOM | 5738 | CD1 | ILE D | 275 | 92.026 | 11.404 | 20.207 | 1.00 | 19.75 | D |
| ATOM | 5739 | C | ILE D | 275 | 90.738 | 13.764 | 16.748 | 1.00 | 26.48 | D |
| ATOM | 5740 | O | ILE D | 275 | 91.490 | 14.743 | 16.855 | 1.00 | 26.90 | D |
| ATOM | 5741 | N | THR D | 276 | 90.052 | 13.503 | 15.642 | 1.00 | 24.10 | D |
| ATOM | 5742 | CA | THR D | 276 | 90.134 | 14.348 | 14.469 | 1.00 | 22.65 | D |
| ATOM | 5743 | CB | THR D | 276 | 89.447 | 13.632 | 13.273 | 1.00 | 21.40 | D |
| ATOM | 5744 | OG1 | THR D | 276 | 90.221 | 12.483 | 12.942 | 1.00 | 21.35 | D |
| ATOM | 5745 | CG2 | THR D | 276 | 89.321 | 14.528 | 12.052 | 1.00 | 18.26 | D |
| ATOM | 5746 | C | THR D | 276 | 89.477 | 15.711 | 14.769 | 1.00 | 23.50 | D |
| ATOM | 5747 | O | THR D | 276 | 90.041 | 16.768 | 14.462 | 1.00 | 22.31 | D |
| ATOM | 5748 | N | SER D | 277 | 88.305 | 15.673 | 15.397 | 1.00 | 23.60 | D |
| ATOM | 5749 | CA | SER D | 277 | 87.582 | 16.883 | 15.745 | 1.00 | 25.64 | D |
| ATOM | 5750 | CB | SER D | 277 | 86.156 | 16.544 | 16.144 | 1.00 | 25.38 | D |
| ATOM | 5751 | OG | SER D | 277 | 85.440 | 16.087 | 15.019 | 1.00 | 27.48 | D |
| ATOM | 5752 | C | SER D | 277 | 88.215 | 17.739 | 16.837 | 1.00 | 26.65 | D |
| ATOM | 5753 | O | SER D | 277 | 88.380 | 18.939 | 16.657 | 1.00 | 29.61 | D |
| ATOM | 5754 | N | LEU D | 278 | 88.561 | 17.147 | 17.970 | 1.00 | 25.91 | D |
| ATOM | 5755 | CA | LEU D | 278 | 89.180 | 17.922 | 19.035 | 1.00 | 26.38 | D |
| ATOM | 5756 | CB | LEU D | 278 | 89.564 | 17.027 | 20.218 | 1.00 | 26.10 | D |
| ATOM | 5757 | CG | LEU D | 278 | 88.426 | 16.811 | 21.194 | 1.00 | 24.98 | D |
| ATOM | 5758 | CD1 | LEU D | 278 | 88.017 | 18.153 | 21.792 | 1.00 | 26.21 | D |
| ATOM | 5759 | CD2 | LEU D | 278 | 87.257 | 16.190 | 20.458 | 1.00 | 28.73 | D |
| ATOM | 5760 | C | LEU D | 278 | 90.426 | 18.621 | 18.530 | 1.00 | 26.46 | D |
| ATOM | 5761 | O | LEU D | 278 | 91.038 | 18.189 | 17.549 | 1.00 | 27.90 | D |
| ATOM | 5762 | N | PRO D | 279 | 90.803 | 19.733 | 19.177 | 1.00 | 26.62 | D |
| ATOM | 5763 | CD | PRO D | 279 | 89.970 | 20.521 | 20.096 | 1.00 | 26.92 | D |
| ATOM | 5764 | CA | PRO D | 279 | 91.994 | 20.496 | 18.799 | 1.00 | 27.49 | D |
| ATOM | 5765 | CB | PRO D | 279 | 91.940 | 21.686 | 19.739 | 1.00 | 26.12 | D |
| ATOM | 5766 | CG | PRO D | 279 | 90.495 | 21.922 | 19.861 | 1.00 | 25.59 | D |
| ATOM | 5767 | C | PRO D | 279 | 93.212 | 19.620 | 19.047 | 1.00 | 29.22 | D |
| ATOM | 5768 | O | PRO D | 279 | 93.221 | 18.827 | 19.991 | 1.00 | 30.55 | D |
| ATOM | 5769 | N | PHE D | 280 | 94.234 | 19.775 | 18.210 | 1.00 | 29.38 | D |
| ATOM | 5770 | CA | PHE D | 280 | 95.452 | 18.980 | 18.303 | 1.00 | 29.08 | D |
| ATOM | 5771 | CB | PHE D | 280 | 96.567 | 19.668 | 17.518 | 1.00 | 30.38 | D |
| ATOM | 5772 | CG | PHE D | 280 | 97.839 | 18.871 | 17.438 | 1.00 | 33.85 | D |
| ATOM | 5773 | CD1 | PHE D | 280 | 97.849 | 17.585 | 16.890 | 1.00 | 35.64 | D |
| ATOM | 5774 | CD2 | PHE D | 280 | 99.038 | 19.415 | 17.886 | 1.00 | 34.62 | D |
| ATOM | 5775 | CE1 | PHE D | 280 | 99.045 | 16.852 | 16.789 | 1.00 | 36.28 | D |
| ATOM | 5776 | CE2 | PHE D | 280 | 100.237 | 18.696 | 17.790 | 1.00 | 35.86 | D |
| ATOM | 5777 | CZ | PHE D | 280 | 100.242 | 17.410 | 17.239 | 1.00 | 36.27 | D |
| ATOM | 5778 | C | PHE D | 280 | 95.883 | 18.747 | 19.746 | 1.00 | 28.87 | D |
| ATOM | 5779 | O | PHE D | 280 | 96.100 | 17.611 | 20.167 | 1.00 | 28.22 | D |
| ATOM | 5780 | N | GLU D | 281 | 95.977 | 19.824 | 20.513 | 1.00 | 29.70 | D |
| ATOM | 5781 | CA | GLU D | 281 | 96.394 | 19.735 | 21.906 | 1.00 | 30.73 | D |
| ATOM | 5782 | CB | GLU D | 281 | 96.367 | 21.124 | 22.544 | 1.00 | 30.74 | D |
| ATOM | 5783 | CG | GLU D | 281 | 96.958 | 22.225 | 21.686 | 1.00 | 35.82 | D |
| ATOM | 5784 | CD | GLU D | 281 | 96.181 | 22.455 | 20.365 | 1.00 | 39.11 | D |
| ATOM | 5785 | OE1 | GLU D | 281 | 94.927 | 22.371 | 20.364 | 1.00 | 37.33 | D |
| ATOM | 5786 | OE2 | GLU D | 281 | 96.838 | 22.734 | 19.329 | 1.00 | 41.78 | D |
| ATOM | 5787 | C | GLU D | 281 | 95.508 | 18.767 | 22.707 | 1.00 | 30.47 | D |
| ATOM | 5788 | O | GLU D | 281 | 95.998 | 17.800 | 23.291 | 1.00 | 29.60 | D |
| ATOM | 5789 | N | ILE D | 282 | 94.200 | 19.006 | 22.731 | 1.00 | 30.62 | D |
| ATOM | 5790 | CA | ILE D | 282 | 93.326 | 18.115 | 23.495 | 1.00 | 30.51 | D |
| ATOM | 5791 | CB | ILE D | 282 | 91.826 | 18.479 | 23.367 | 1.00 | 28.68 | D |
| ATOM | 5792 | CG2 | ILE D | 282 | 90.973 | 17.454 | 24.096 | 1.00 | 25.40 | D |
| ATOM | 5793 | CG1 | ILE D | 282 | 91.559 | 19.841 | 24.002 | 1.00 | 28.37 | D |
| ATOM | 5794 | CD1 | ILE D | 282 | 92.338 | 20.960 | 23.374 | 1.00 | 29.50 | D |
| ATOM | 5795 | C | ILE D | 282 | 93.506 | 16.667 | 23.085 | 1.00 | 30.93 | D |
| ATOM | 5796 | O | ILE D | 282 | 93.569 | 15.798 | 23.939 | 1.00 | 32.03 | D |
| ATOM | 5797 | N | SER D | 283 | 93.597 | 16.403 | 21.786 | 1.00 | 33.38 | D |
| ATOM | 5798 | CA | SER D | 283 | 93.773 | 15.037 | 21.315 | 1.00 | 33.57 | D |
| ATOM | 5799 | CB | SER D | 283 | 93.729 | 14.991 | 19.793 | 1.00 | 33.09 | D |
| ATOM | 5800 | OG | SER D | 283 | 92.440 | 14.625 | 19.343 | 1.00 | 33.69 | D |
| ATOM | 5801 | C | SER D | 283 | 95.060 | 14.383 | 21.806 | 1.00 | 34.43 | D |
| ATOM | 5802 | O | SER D | 283 | 95.102 | 13.171 | 21.996 | 1.00 | 35.71 | D |
| ATOM | 5803 | N | LEU D | 284 | 96.121 | 15.158 | 21.996 | 1.00 | 33.67 | D |
| ATOM | 5804 | CA | LEU D | 284 | 97.345 | 14.541 | 22.479 | 1.00 | 33.99 | D |
| ATOM | 5805 | CB | LEU D | 284 | 98.511 | 15.514 | 22.412 | 1.00 | 34.78 | D |
| ATOM | 5806 | CG | LEU D | 284 | 98.908 | 15.828 | 20.966 | 1.00 | 37.92 | D |
| ATOM | 5807 | CD1 | LEU D | 284 | 99.965 | 16.921 | 20.972 | 1.00 | 38.44 | D |
| ATOM | 5808 | CD2 | LEU D | 284 | 99.424 | 14.571 | 20.264 | 1.00 | 35.81 | D |
| ATOM | 5809 | C | LEU D | 284 | 97.105 | 14.089 | 23.908 | 1.00 | 34.14 | D |
| ATOM | 5810 | O | LEU D | 284 | 97.469 | 12.972 | 24.268 | 1.00 | 35.96 | D |
| ATOM | 5811 | N | LYS D | 285 | 96.470 | 14.944 | 24.710 | 1.00 | 32.83 | D |
| ATOM | 5812 | CA | LYS D | 285 | 96.180 | 14.598 | 26.094 | 1.00 | 31.02 | D |
| ATOM | 5813 | CB | LYS D | 285 | 95.192 | 15.590 | 26.711 | 1.00 | 32.29 | D |
| ATOM | 5814 | CG | LYS D | 285 | 95.694 | 17.027 | 26.672 | 1.00 | 34.86 | D |
| ATOM | 5815 | CD | LYS D | 285 | 94.977 | 17.944 | 27.659 | 1.00 | 38.81 | D |
| ATOM | 5816 | CE | LYS D | 285 | 95.626 | 17.931 | 29.043 | 1.00 | 41.32 | D |
| ATOM | 5817 | NZ | LYS D | 285 | 95.083 | 19.055 | 29.866 | 1.00 | 43.73 | D |
| ATOM | 5818 | C | LYS D | 285 | 95.603 | 13.194 | 26.129 | 1.00 | 28.79 | D |
| ATOM | 5819 | O | LYS D | 285 | 96.124 | 12.305 | 26.823 | 1.00 | 29.62 | D |
| ATOM | 5820 | N | ILE D | 286 | 94.538 | 12.991 | 25.360 | 1.00 | 25.19 | D |
| ATOM | 5821 | CA | ILE D | 286 | 93.898 | 11.681 | 25.281 | 1.00 | 24.05 | D |
| ATOM | 5822 | CB | ILE D | 286 | 92.834 | 11.660 | 24.148 | 1.00 | 22.82 | D |
| ATOM | 5823 | CG2 | ILE D | 286 | 92.176 | 10.291 | 24.060 | 1.00 | 21.24 | D |
| ATOM | 5824 | CG1 | ILE D | 286 | 91.800 | 12.757 | 24.414 | 1.00 | 23.94 | D |
| ATOM | 5825 | CD1 | ILE D | 286 | 90.731 | 12.886 | 23.321 | 1.00 | 23.24 | D |
| ATOM | 5826 | C | ILE D | 286 | 94.950 | 10.583 | 25.036 | 1.00 | 23.79 | D |
| ATOM | 5827 | O | ILE D | 286 | 94.970 | 9.573 | 25.738 | 1.00 | 22.04 | D |
| ATOM | 5828 | N | PHE D | 287 | 95.825 | 10.788 | 24.051 | 1.00 | 21.07 | D |
| ATOM | 5829 | CA | PHE D | 287 | 96.844 | 9.790 | 23.746 | 1.00 | 21.11 | D |
| ATOM | 5830 | CB | PHE D | 287 | 97.596 | 10.147 | 22.469 | 1.00 | 20.53 | D |
| ATOM | 5831 | CG | PHE D | 287 | 96.842 | 9.818 | 21.221 | 1.00 | 20.94 | D |
| ATOM | 5832 | CD1 | PHE D | 287 | 96.528 | 8.495 | 20.915 | 1.00 | 18.61 | D |
| ATOM | 5833 | CD2 | PHE D | 287 | 96.445 | 10.831 | 20.340 | 1.00 | 20.85 | D |
| ATOM | 5834 | CE1 | PHE D | 287 | 95.835 | 8.184 | 19.754 | 1.00 | 17.26 | D |
| ATOM | 5835 | CE2 | PHE D | 287 | 95.752 | 10.525 | 19.177 | 1.00 | 18.21 | D |
| ATOM | 5836 | CZ | PHE D | 287 | 95.444 | 9.204 | 18.876 | 1.00 | 16.07 | D |
| ATOM | 5837 | C | PHE D | 287 | 97.849 | 9.539 | 24.859 | 1.00 | 21.96 | D |
| ATOM | 5838 | O | PHE D | 287 | 98.314 | 8.418 | 25.035 | 1.00 | 20.26 | D |
| ATOM | 5839 | N | ASN D | 288 | 98.182 | 10.592 | 25.593 | 1.00 | 23.06 | D |
| ATOM | 5840 | CA | ASN D | 288 | 99.128 | 10.494 | 26.677 | 1.00 | 25.24 | D |
| ATOM | 5841 | CB | ASN D | 288 | 99.598 | 11.892 | 27.089 | 1.00 | 25.50 | D |
| ATOM | 5842 | CG | ASN D | 288 | 100.691 | 12.432 | 26.166 | 1.00 | 27.45 | D |
| ATOM | 5843 | OD1 | ASN D | 288 | 101.539 | 11.660 | 25.680 | 1.00 | 29.10 | D |
| ATOM | 5844 | ND2 | ASN D | 288 | 100.696 | 13.750 | 25.936 | 1.00 | 25.61 | D |
| ATOM | 5845 | C | ASN D | 288 | 98.585 | 9.695 | 27.883 | 1.00 | 28.89 | D |
| ATOM | 5846 | O | ASN D | 288 | 99.331 | 9.376 | 28.815 | 1.00 | 28.16 | D |
| ATOM | 5847 | N | TYR D | 289 | 97.289 | 9.368 | 27.846 | 1.00 | 30.81 | D |
| ATOM | 5848 | CA | TYR D | 289 | 96.633 | 8.560 | 28.879 | 1.00 | 33.41 | D |
| ATOM | 5849 | CB | TYR D | 289 | 95.210 | 9.038 | 29.193 | 1.00 | 35.09 | D |
| ATOM | 5850 | CG | TYR D | 289 | 95.100 | 10.237 | 30.098 | 1.00 | 38.19 | D |
| ATOM | 5851 | CD1 | TYR D | 289 | 95.713 | 10.256 | 31.343 | 1.00 | 37.23 | D |
| ATOM | 5852 | CE1 | TYR D | 289 | 95.585 | 11.353 | 32.182 | 1.00 | 37.83 | D |
| ATOM | 5853 | CD2 | TYR D | 289 | 94.353 | 11.348 | 29.713 | 1.00 | 38.71 | D |
| ATOM | 5854 | CE2 | TYR D | 289 | 94.219 | 12.447 | 30.543 | 1.00 | 39.19 | D |
| ATOM | 5855 | CZ | TYR D | 289 | 94.838 | 12.449 | 31.778 | 1.00 | 38.07 | D |
| ATOM | 5856 | OH | TYR D | 289 | 94.716 | 13.558 | 32.596 | 1.00 | 37.65 | D |
| ATOM | 5857 | C | TYR D | 289 | 96.496 | 7.129 | 28.375 | 1.00 | 34.42 | D |
| ATOM | 5858 | O | TYR D | 289 | 95.852 | 6.288 | 29.016 | 1.00 | 34.85 | D |
| ATOM | 5859 | N | LEU D | 290 | 97.076 | 6.857 | 27.215 | 1.00 | 34.95 | D |
| ATOM | 5860 | CA | LEU D | 290 | 96.980 | 5.530 | 26.632 | 1.00 | 36.71 | D |
| ATOM | 5861 | CB | LEU D | 290 | 96.488 | 5.621 | 25.189 | 1.00 | 37.91 | D |
| ATOM | 5862 | CG | LEU D | 290 | 95.004 | 5.925 | 24.991 | 1.00 | 38.82 | D |
| ATOM | 5863 | CD1 | LEU D | 290 | 94.740 | 6.136 | 23.516 | 1.00 | 39.93 | D |
| ATOM | 5864 | CD2 | LEU D | 290 | 94.159 | 4.764 | 25.535 | 1.00 | 37.72 | D |
| ATOM | 5865 | C | LEU D | 290 | 98.302 | 4.802 | 26.664 | 1.00 | 37.19 | D |
| ATOM | 5866 | O | LEU D | 290 | 99.354 | 5.419 | 26.628 | 1.00 | 37.91 | D |
| ATOM | 5867 | N | GLN D | 291 | 98.256 | 3.482 | 26.744 | 1.00 | 38.53 | D |
| ATOM | 5868 | CA | GLN D | 291 | 99.493 | 2.736 | 26.771 | 1.00 | 39.34 | D |
| ATOM | 5869 | CB | GLN D | 291 | 99.251 | 1.308 | 27.236 | 1.00 | 40.51 | D |
| ATOM | 5870 | CG | GLN D | 291 | 98.786 | 1.213 | 28.657 | 1.00 | 43.34 | D |
| ATOM | 5871 | CD | GLN D | 291 | 98.666 | −0.215 | 29.102 | 1.00 | 46.99 | D |
| ATOM | 5872 | OE1 | GLN D | 291 | 99.662 | −0.945 | 29.172 | 1.00 | 50.86 | D |
| ATOM | 5873 | NE2 | GLN D | 291 | 97.444 | −0.638 | 29.398 | 1.00 | 49.26 | D |
| ATOM | 5874 | C | GLN D | 291 | 100.090 | 2.749 | 25.374 | 1.00 | 38.68 | D |
| ATOM | 5875 | O | GLN D | 291 | 99.393 | 2.990 | 24.383 | 1.00 | 39.01 | D |
| ATOM | 5876 | N | PHE D | 292 | 101.386 | 2.482 | 25.308 | 1.00 | 35.62 | D |
| ATOM | 5877 | CA | PHE D | 292 | 102.096 | 2.492 | 24.059 | 1.00 | 32.95 | D |
| ATOM | 5878 | CB | PHE D | 292 | 103.583 | 2.209 | 24.335 | 1.00 | 32.02 | D |
| ATOM | 5879 | CG | PHE D | 292 | 104.010 | 0.795 | 24.109 | 1.00 | 29.67 | D |
| ATOM | 5880 | CD1 | PHE D | 292 | 104.290 | 0.349 | 22.829 | 1.00 | 30.05 | D |
| ATOM | 5881 | CD2 | PHE D | 292 | 104.221 | −0.064 | 25.176 | 1.00 | 29.27 | D |
| ATOM | 5882 | CE1 | PHE D | 292 | 104.788 | −0.932 | 22.613 | 1.00 | 30.66 | D |
| ATOM | 5883 | CE2 | PHE D | 292 | 104.719 | −1.348 | 24.972 | 1.00 | 29.01 | D |
| ATOM | 5884 | CZ | PHE D | 292 | 105.004 | −1.781 | 23.686 | 1.00 | 29.86 | D |
| ATOM | 5885 | C | PHE D | 292 | 101.477 | 1.552 | 23.035 | 1.00 | 32.44 | D |
| ATOM | 5886 | O | PHE D | 292 | 101.384 | 1.894 | 21.852 | 1.00 | 33.62 | D |
| ATOM | 5887 | N | GLU D | 293 | 101.018 | 0.389 | 23.481 | 1.00 | 31.32 | D |
| ATOM | 5888 | CA | GLU D | 293 | 100.396 | −0.551 | 22.560 | 1.00 | 29.89 | D |
| ATOM | 5889 | CB | GLU D | 293 | 99.974 | −1.819 | 23.296 | 1.00 | 29.10 | D |
| ATOM | 5890 | CG | GLU D | 293 | 101.135 | −2.712 | 23.586 | 1.00 | 30.44 | D |
| ATOM | 5891 | CD | GLU D | 293 | 101.534 | −2.717 | 25.053 | 1.00 | 31.93 | D |
| ATOM | 5892 | OE1 | GLU D | 293 | 101.473 | −1.647 | 25.722 | 1.00 | 28.98 | D |
| ATOM | 5893 | OE2 | GLU D | 293 | 101.924 | −3.815 | 25.518 | 1.00 | 32.42 | D |
| ATOM | 5894 | C | GLU D | 293 | 99.187 | 0.044 | 21.864 | 1.00 | 28.52 | D |
| ATOM | 5895 | O | GLU D | 293 | 98.978 | −0.193 | 20.679 | 1.00 | 26.63 | D |
| ATOM | 5896 | N | ASP D | 294 | 98.391 | 0.802 | 22.615 | 1.00 | 29.50 | D |
| ATOM | 5897 | CA | ASP D | 294 | 97.193 | 1.431 | 22.081 | 1.00 | 30.32 | D |
| ATOM | 5898 | CB | ASP D | 294 | 96.330 | 2.030 | 23.210 | 1.00 | 32.63 | D |
| ATOM | 5899 | CG | ASP D | 294 | 95.768 | 0.968 | 24.183 | 1.00 | 36.82 | D |
| ATOM | 5900 | OD1 | ASP D | 294 | 95.503 | −0.192 | 23.772 | 1.00 | 39.44 | D |
| ATOM | 5901 | OD2 | ASP D | 294 | 95.563 | 1.309 | 25.369 | 1.00 | 38.44 | D |
| ATOM | 5902 | C | ASP D | 294 | 97.585 | 2.542 | 21.103 | 1.00 | 29.40 | D |
| ATOM | 5903 | O | ASP D | 294 | 96.796 | 2.930 | 20.245 | 1.00 | 27.84 | D |
| ATOM | 5904 | N | ILE D | 295 | 98.805 | 3.050 | 21.237 | 1.00 | 28.17 | D |
| ATOM | 5905 | CA | ILE D | 295 | 99.266 | 4.114 | 20.362 | 1.00 | 28.34 | D |
| ATOM | 5906 | CB | ILE D | 295 | 100.510 | 4.784 | 20.917 | 1.00 | 28.05 | D |
| ATOM | 5907 | CG2 | ILE D | 295 | 100.845 | 5.998 | 20.077 | 1.00 | 25.51 | D |
| ATOM | 5908 | CG1 | ILE D | 295 | 100.294 | 5.145 | 22.392 | 1.00 | 30.16 | D |
| ATOM | 5909 | CD1 | ILE D | 295 | 99.338 | 6.283 | 22.658 | 1.00 | 31.34 | D |
| ATOM | 5910 | C | ILE D | 295 | 99.605 | 3.549 | 18.990 | 1.00 | 28.98 | D |
| ATOM | 5911 | O | ILE D | 295 | 99.310 | 4.151 | 17.956 | 1.00 | 28.75 | D |
| ATOM | 5912 | N | ILE D | 296 | 100.227 | 2.381 | 18.996 | 1.00 | 29.17 | D |
| ATOM | 5913 | CA | ILE D | 296 | 100.612 | 1.715 | 17.768 | 1.00 | 30.36 | D |
| ATOM | 5914 | CB | ILE D | 296 | 101.521 | 0.491 | 18.085 | 1.00 | 30.31 | D |
| ATOM | 5915 | CG2 | ILE D | 296 | 101.236 | −0.660 | 17.148 | 1.00 | 28.53 | D |
| ATOM | 5916 | CG1 | ILE D | 296 | 102.987 | 0.911 | 17.989 | 1.00 | 31.04 | D |
| ATOM | 5917 | CD1 | ILE D | 296 | 103.353 | 2.115 | 18.829 | 1.00 | 32.07 | D |
| ATOM | 5918 | C | ILE D | 296 | 99.379 | 1.308 | 16.965 | 1.00 | 30.74 | D |
| ATOM | 5919 | O | ILE D | 296 | 99.376 | 1.398 | 15.741 | 1.00 | 30.48 | D |
| ATOM | 5920 | N | ASN D | 297 | 98.330 | 0.865 | 17.642 | 1.00 | 31.12 | D |
| ATOM | 5921 | CA | ASN D | 297 | 97.121 | 0.506 | 16.924 | 1.00 | 33.08 | D |
| ATOM | 5922 | CB | ASN D | 297 | 96.096 | −0.099 | 17.865 | 1.00 | 33.38 | D |
| ATOM | 5923 | CG | ASN D | 297 | 96.393 | −1.537 | 18.171 | 1.00 | 37.25 | D |
| ATOM | 5924 | OD1 | ASN D | 297 | 95.953 | −2.067 | 19.194 | 1.00 | 37.85 | D |
| ATOM | 5925 | ND2 | ASN D | 297 | 97.143 | −2.196 | 17.270 | 1.00 | 37.30 | D |
| ATOM | 5926 | C | ASN D | 297 | 96.548 | 1.767 | 16.316 | 1.00 | 33.81 | D |
| ATOM | 5927 | O | ASN D | 297 | 96.220 | 1.817 | 15.123 | 1.00 | 34.30 | D |
| ATOM | 5928 | N | SER D | 298 | 96.435 | 2.780 | 17.168 | 1.00 | 32.14 | D |
| ATOM | 5929 | CA | SER D | 298 | 95.912 | 4.079 | 16.792 | 1.00 | 30.45 | D |
| ATOM | 5930 | CB | SER D | 298 | 96.100 | 5.073 | 17.939 | 1.00 | 29.96 | D |
| ATOM | 5931 | OG | SER D | 298 | 95.395 | 4.648 | 19.087 | 1.00 | 27.42 | D |
| ATOM | 5932 | C | SER D | 298 | 96.656 | 4.566 | 15.583 | 1.00 | 30.58 | D |
| ATOM | 5933 | O | SER D | 298 | 96.103 | 5.255 | 14.731 | 1.00 | 32.34 | D |
| ATOM | 5934 | N | LEU D | 299 | 97.930 | 4.216 | 15.514 | 1.00 | 29.49 | D |
| ATOM | 5935 | CA | LEU D | 299 | 98.738 | 4.628 | 14.387 | 1.00 | 28.00 | D |
| ATOM | 5936 | CB | LEU D | 299 | 100.164 | 4.131 | 14.570 | 1.00 | 28.12 | D |
| ATOM | 5937 | CG | LEU D | 299 | 101.291 | 5.098 | 14.240 | 1.00 | 28.83 | D |
| ATOM | 5938 | CD1 | LEU D | 299 | 101.105 | 6.442 | 14.938 | 1.00 | 28.49 | D |
| ATOM | 5939 | CD2 | LEU D | 299 | 102.595 | 4.455 | 14.703 | 1.00 | 30.72 | D |
| ATOM | 5940 | C | LEU D | 299 | 98.147 | 4.063 | 13.108 | 1.00 | 27.04 | D |
| ATOM | 5941 | O | LEU D | 299 | 98.382 | 4.608 | 12.045 | 1.00 | 28.76 | D |
| ATOM | 5942 | N | GLY D | 300 | 97.356 | 2.996 | 13.212 | 1.00 | 25.60 | D |
| ATOM | 5943 | CA | GLY D | 300 | 96.783 | 2.387 | 12.018 | 1.00 | 25.16 | D |
| ATOM | 5944 | C | GLY D | 300 | 95.268 | 2.423 | 11.857 | 1.00 | 25.49 | D |
| ATOM | 5945 | O | GLY D | 300 | 94.665 | 1.528 | 11.258 | 1.00 | 24.96 | D |
| ATOM | 5946 | N | VAL D | 301 | 94.661 | 3.476 | 12.381 | 1.00 | 26.14 | D |
| ATOM | 5947 | CA | VAL D | 301 | 93.223 | 3.677 | 12.335 | 1.00 | 25.65 | D |
| ATOM | 5948 | CB | VAL D | 301 | 92.783 | 4.425 | 13.635 | 1.00 | 25.13 | D |
| ATOM | 5949 | CG1 | VAL D | 301 | 91.377 | 4.975 | 13.523 | 1.00 | 23.49 | D |
| ATOM | 5950 | CG2 | VAL D | 301 | 92.881 | 3.470 | 14.818 | 1.00 | 25.09 | D |
| ATOM | 5951 | C | VAL D | 301 | 92.853 | 4.482 | 11.085 | 1.00 | 26.83 | D |
| ATOM | 5952 | O | VAL D | 301 | 91.895 | 4.160 | 10.386 | 1.00 | 29.06 | D |
| ATOM | 5953 | N | SER D | 302 | 93.624 | 5.517 | 10.781 | 1.00 | 27.15 | D |
| ATOM | 5954 | CA | SER D | 302 | 93.321 | 6.327 | 9.611 | 1.00 | 27.14 | D |
| ATOM | 5955 | CB | SER D | 302 | 92.021 | 7.096 | 9.832 | 1.00 | 26.96 | D |
| ATOM | 5956 | OG | SER D | 302 | 92.186 | 8.048 | 10.876 | 1.00 | 28.29 | D |
| ATOM | 5957 | C | SER D | 302 | 94.442 | 7.308 | 9.329 | 1.00 | 26.93 | D |
| ATOM | 5958 | O | SER D | 302 | 95.174 | 7.696 | 10.239 | 1.00 | 27.64 | D |
| ATOM | 5959 | N | GLN D | 303 | 94.558 | 7.720 | 8.067 | 1.00 | 26.82 | D |
| ATOM | 5960 | CA | GLN D | 303 | 95.596 | 8.658 | 7.661 | 1.00 | 25.04 | D |
| ATOM | 5961 | CB | GLN D | 303 | 95.362 | 9.166 | 6.244 | 1.00 | 23.83 | D |
| ATOM | 5962 | CG | GLN D | 303 | 95.865 | 8.261 | 5.154 | 1.00 | 29.99 | D |
| ATOM | 5963 | CD | GLN D | 303 | 97.398 | 8.160 | 5.090 | 1.00 | 32.84 | D |
| ATOM | 5964 | OE1 | GLN D | 303 | 97.942 | 7.457 | 4.239 | 1.00 | 34.49 | D |
| ATOM | 5965 | NE2 | GLN D | 303 | 98.091 | 8.863 | 5.986 | 1.00 | 36.06 | D |
| ATOM | 5966 | C | GLN D | 303 | 95.653 | 9.852 | 8.587 | 1.00 | 23.97 | D |
| ATOM | 5967 | O | GLN D | 303 | 96.743 | 10.343 | 8.901 | 1.00 | 22.53 | D |
| ATOM | 5968 | N | ASN D | 304 | 94.490 | 10.322 | 9.034 | 1.00 | 22.85 | D |
| ATOM | 5969 | CA | ASN D | 304 | 94.479 | 11.487 | 9.908 | 1.00 | 24.54 | D |
| ATOM | 5970 | CB | ASN D | 304 | 93.082 | 12.042 | 10.095 | 1.00 | 25.34 | D |
| ATOM | 5971 | CG | ASN D | 304 | 93.105 | 13.358 | 10.814 | 1.00 | 28.24 | D |
| ATOM | 5972 | OD1 | ASN D | 304 | 92.215 | 13.657 | 11.613 | 1.00 | 30.04 | D |
| ATOM | 5973 | ND2 | ASN D | 304 | 94.143 | 14.163 | 10.544 | 1.00 | 29.46 | D |
| ATOM | 5974 | C | ASN D | 304 | 95.058 | 11.172 | 11.270 | 1.00 | 25.25 | D |
| ATOM | 5975 | O | ASN D | 304 | 95.876 | 11.928 | 11.820 | 1.00 | 24.93 | D |
| ATOM | 5976 | N | TRP D | 305 | 94.609 | 10.063 | 11.835 | 1.00 | 25.15 | D |
| ATOM | 5977 | CA | TRP D | 305 | 95.139 | 9.658 | 13.118 | 1.00 | 25.51 | D |
| ATOM | 5978 | CB | TRP D | 305 | 94.447 | 8.373 | 13.587 | 1.00 | 25.43 | D |
| ATOM | 5979 | CG | TRP D | 305 | 93.138 | 8.630 | 14.311 | 1.00 | 26.00 | D |
| ATOM | 5980 | CD2 | TRP D | 305 | 92.614 | 7.880 | 15.410 | 1.00 | 26.49 | D |
| ATOM | 5981 | CE2 | TRP D | 305 | 91.351 | 8.425 | 15.730 | 1.00 | 24.97 | D |
| ATOM | 5982 | CE3 | TRP D | 305 | 93.090 | 6.796 | 16.157 | 1.00 | 27.62 | D |
| ATOM | 5983 | CD1 | TRP D | 305 | 92.199 | 9.581 | 14.015 | 1.00 | 25.09 | D |
| ATOM | 5984 | NE1 | TRP D | 305 | 91.122 | 9.461 | 14.864 | 1.00 | 24.10 | D |
| ATOM | 5985 | CZ2 | TRP D | 305 | 90.559 | 7.918 | 16.764 | 1.00 | 23.76 | D |
| ATOM | 5986 | CZ3 | TRP D | 305 | 92.299 | 6.297 | 17.183 | 1.00 | 25.00 | D |
| ATOM | 5987 | CH2 | TRP D | 305 | 91.053 | 6.859 | 17.474 | 1.00 | 24.03 | D |
| ATOM | 5988 | C | TRP D | 305 | 96.656 | 9.477 | 12.935 | 1.00 | 24.69 | D |
| ATOM | 5989 | O | TRP D | 305 | 97.445 | 9.992 | 13.734 | 1.00 | 25.68 | D |
| ATOM | 5990 | N | ASN D | 306 | 97.054 | 8.786 | 11.863 | 1.00 | 22.23 | D |
| ATOM | 5991 | CA | ASN D | 306 | 98.471 | 8.583 | 11.555 | 1.00 | 21.36 | D |
| ATOM | 5992 | CB | ASN D | 306 | 98.669 | 7.946 | 10.182 | 1.00 | 19.88 | D |
| ATOM | 5993 | CG | ASN D | 306 | 100.134 | 7.572 | 9.920 | 1.00 | 20.62 | D |
| ATOM | 5994 | OD1 | ASN D | 306 | 100.608 | 6.526 | 10.394 | 1.00 | 21.63 | D |
| ATOM | 5995 | ND2 | ASN D | 306 | 100.860 | 8.427 | 9.176 | 1.00 | 16.92 | D |
| ATOM | 5996 | C | ASN D | 306 | 99.203 | 9.913 | 11.517 | 1.00 | 21.93 | D |
| ATOM | 5997 | O | ASN D | 306 | 100.356 | 10.009 | 11.912 | 1.00 | 21.70 | D |
| ATOM | 5998 | N | LYS D | 307 | 98.533 | 10.930 | 11.000 | 1.00 | 22.01 | D |
| ATOM | 5999 | CA | LYS D | 307 | 99.134 | 12.235 | 10.907 | 1.00 | 23.12 | D |
| ATOM | 6000 | CB | LYS D | 307 | 98.270 | 13.136 | 10.027 | 1.00 | 24.42 | D |
| ATOM | 6001 | CG | LYS D | 307 | 98.897 | 14.469 | 9.657 | 1.00 | 25.07 | D |
| ATOM | 6002 | CD | LYS D | 307 | 97.803 | 15.504 | 9.446 | 1.00 | 29.41 | D |
| ATOM | 6003 | CE | LYS D | 307 | 98.290 | 16.723 | 8.650 | 1.00 | 31.20 | D |
| ATOM | 6004 | NZ | LYS D | 307 | 98.710 | 16.351 | 7.251 | 1.00 | 30.25 | D |
| ATOM | 6005 | C | LYS D | 307 | 99.298 | 12.868 | 12.284 | 1.00 | 24.28 | D |
| ATOM | 6006 | O | LYS D | 307 | 100.387 | 13.292 | 12.654 | 1.00 | 26.10 | D |
| ATOM | 6007 | N | ILE D | 308 | 98.214 | 12.902 | 13.047 | 1.00 | 23.74 | D |
| ATOM | 6008 | CA | ILE D | 308 | 98.217 | 13.520 | 14.359 | 1.00 | 25.31 | D |
| ATOM | 6009 | CB | ILE D | 308 | 96.843 | 13.350 | 15.042 | 1.00 | 27.23 | D |
| ATOM | 6010 | CG2 | ILE D | 308 | 96.899 | 13.810 | 16.475 | 1.00 | 27.28 | D |
| ATOM | 6011 | CG1 | ILE D | 308 | 95.794 | 14.187 | 14.294 | 1.00 | 28.70 | D |
| ATOM | 6012 | CD1 | ILE D | 308 | 94.383 | 14.048 | 14.808 | 1.00 | 26.95 | D |
| ATOM | 6013 | C | ILE D | 308 | 99.298 | 13.018 | 15.292 | 1.00 | 25.96 | D |
| ATOM | 6014 | O | ILE D | 308 | 100.079 | 13.797 | 15.838 | 1.00 | 24.81 | D |
| ATOM | 6015 | N | ILE D | 309 | 99.353 | 11.710 | 15.464 | 1.00 | 26.12 | D |
| ATOM | 6016 | CA | ILE D | 309 | 100.314 | 11.115 | 16.359 | 1.00 | 25.75 | D |
| ATOM | 6017 | CB | ILE D | 309 | 99.990 | 9.603 | 16.538 | 1.00 | 25.30 | D |
| ATOM | 6018 | CG2 | ILE D | 309 | 100.995 | 8.937 | 17.437 | 1.00 | 24.21 | D |
| ATOM | 6019 | CG1 | ILE D | 309 | 98.617 | 9.437 | 17.181 | 1.00 | 21.52 | D |
| ATOM | 6020 | CD1 | ILE D | 309 | 98.160 | 8.023 | 17.261 | 1.00 | 23.28 | D |
| ATOM | 6021 | C | ILE D | 309 | 101.755 | 11.313 | 15.906 | 1.00 | 26.74 | D |
| ATOM | 6022 | O | ILE D | 309 | 102.626 | 11.586 | 16.729 | 1.00 | 27.84 | D |
| ATOM | 6023 | N | ARG D | 310 | 102.026 | 11.177 | 14.610 | 1.00 | 28.38 | D |
| ATOM | 6024 | CA | ARG D | 310 | 103.401 | 11.350 | 14.098 | 1.00 | 29.18 | D |
| ATOM | 6025 | CB | ARG D | 310 | 103.513 | 10.811 | 12.663 | 1.00 | 28.52 | D |
| ATOM | 6026 | CG | ARG D | 310 | 103.531 | 9.277 | 12.560 | 1.00 | 30.67 | D |
| ATOM | 6027 | CD | ARG D | 310 | 103.052 | 8.787 | 11.186 | 1.00 | 34.25 | D |
| ATOM | 6028 | NE | ARG D | 310 | 104.098 | 8.252 | 10.323 | 1.00 | 36.16 | D |
| ATOM | 6029 | CZ | ARG D | 310 | 105.097 | 8.969 | 9.827 | 1.00 | 38.55 | D |
| ATOM | 6030 | NH1 | ARG D | 310 | 105.200 | 10.259 | 10.095 | 1.00 | 42.20 | D |
| ATOM | 6031 | NH2 | ARG D | 310 | 106.009 | 8.391 | 9.069 | 1.00 | 41.89 | D |
| ATOM | 6032 | C | ARG D | 310 | 103.838 | 12.811 | 14.139 | 1.00 | 28.88 | D |
| ATOM | 6033 | O | ARG D | 310 | 104.996 | 13.133 | 13.873 | 1.00 | 28.32 | D |
| ATOM | 6034 | N | LYS D | 311 | 102.900 | 13.681 | 14.496 | 1.00 | 29.76 | D |
| ATOM | 6035 | CA | LYS D | 311 | 103.172 | 15.104 | 14.570 | 1.00 | 32.11 | D |
| ATOM | 6036 | CB | LYS D | 311 | 101.894 | 15.868 | 14.227 | 1.00 | 32.03 | D |
| ATOM | 6037 | CG | LYS D | 311 | 102.061 | 17.362 | 14.098 | 1.00 | 36.15 | D |
| ATOM | 6038 | CD | LYS D | 311 | 100.731 | 18.049 | 13.814 | 1.00 | 39.43 | D |
| ATOM | 6039 | CE | LYS D | 311 | 100.895 | 19.580 | 13.814 | 1.00 | 42.31 | D |
| ATOM | 6040 | NZ | LYS D | 311 | 100.575 | 20.231 | 15.122 | 1.00 | 42.00 | D |
| ATOM | 6041 | C | LYS D | 311 | 103.728 | 15.564 | 15.934 | 1.00 | 32.85 | D |
| ATOM | 6042 | O | LYS D | 311 | 104.467 | 16.545 | 15.992 | 1.00 | 32.33 | D |
| ATOM | 6043 | N | SER D | 312 | 103.395 | 14.855 | 17.017 | 1.00 | 33.76 | D |
| ATOM | 6044 | CA | SER D | 312 | 103.857 | 15.223 | 18.365 | 1.00 | 33.60 | D |
| ATOM | 6045 | CB | SER D | 312 | 102.835 | 14.787 | 19.421 | 1.00 | 35.34 | D |
| ATOM | 6046 | OG | SER D | 312 | 103.198 | 15.271 | 20.712 | 1.00 | 39.47 | D |
| ATOM | 6047 | C | SER D | 312 | 105.222 | 14.667 | 18.781 | 1.00 | 31.68 | D |
| ATOM | 6048 | O | SER D | 312 | 105.456 | 13.468 | 18.728 | 1.00 | 32.40 | D |
| ATOM | 6049 | N | THR D | 313 | 106.125 | 15.543 | 19.198 | 1.00 | 30.17 | D |
| ATOM | 6050 | CA | THR D | 313 | 107.429 | 15.095 | 19.651 | 1.00 | 29.28 | D |
| ATOM | 6051 | CB | THR D | 313 | 108.502 | 16.214 | 19.480 | 1.00 | 30.08 | D |
| ATOM | 6052 | OG1 | THR D | 313 | 109.122 | 16.072 | 18.199 | 1.00 | 31.16 | D |
| ATOM | 6053 | CG2 | THR D | 313 | 109.580 | 16.132 | 20.558 | 1.00 | 30.05 | D |
| ATOM | 6054 | C | THR D | 313 | 107.270 | 14.701 | 21.128 | 1.00 | 27.77 | D |
| ATOM | 6055 | O | THR D | 313 | 107.658 | 13.603 | 21.538 | 1.00 | 26.51 | D |
| ATOM | 6056 | N | SER D | 314 | 106.666 | 15.599 | 21.901 | 1.00 | 26.12 | D |
| ATOM | 6057 | CA | SER D | 314 | 106.420 | 15.370 | 23.311 | 1.00 | 26.42 | D |
| ATOM | 6058 | CB | SER D | 314 | 105.499 | 16.454 | 23.886 | 1.00 | 25.86 | D |
| ATOM | 6059 | OG | SER D | 314 | 104.133 | 16.191 | 23.589 | 1.00 | 25.20 | D |
| ATOM | 6060 | C | SER D | 314 | 105.755 | 14.011 | 23.534 | 1.00 | 26.86 | D |
| ATOM | 6061 | O | SER D | 314 | 106.064 | 13.314 | 24.486 | 1.00 | 27.19 | D |
| ATOM | 6062 | N | LEU D | 315 | 104.830 | 13.633 | 22.668 | 1.00 | 26.78 | D |
| ATOM | 6063 | CA | LEU D | 315 | 104.169 | 12.368 | 22.851 | 1.00 | 26.12 | D |
| ATOM | 6064 | CB | LEU D | 315 | 103.148 | 12.167 | 21.741 | 1.00 | 25.84 | D |
| ATOM | 6065 | CG | LEU D | 315 | 102.212 | 10.960 | 21.711 | 1.00 | 26.39 | D |
| ATOM | 6066 | CD1 | LEU D | 315 | 101.094 | 11.269 | 20.721 | 1.00 | 27.77 | D |
| ATOM | 6067 | CD2 | LEU D | 315 | 102.933 | 9.691 | 21.303 | 1.00 | 24.47 | D |
| ATOM | 6068 | C | LEU D | 315 | 105.186 | 11.229 | 22.899 | 1.00 | 26.05 | D |
| ATOM | 6069 | O | LEU D | 315 | 105.108 | 10.373 | 23.780 | 1.00 | 27.85 | D |
| ATOM | 6070 | N | TRP D | 316 | 106.165 | 11.224 | 21.998 | 1.00 | 24.50 | D |
| ATOM | 6071 | CA | TRP D | 316 | 107.141 | 10.138 | 22.011 | 1.00 | 25.89 | D |
| ATOM | 6072 | CB | TRP D | 316 | 107.751 | 9.952 | 20.607 | 1.00 | 24.16 | D |
| ATOM | 6073 | CG | TRP D | 316 | 106.708 | 9.452 | 19.635 | 1.00 | 21.27 | D |
| ATOM | 6074 | CD2 | TRP D | 316 | 106.177 | 8.123 | 19.565 | 1.00 | 18.61 | D |
| ATOM | 6075 | CE2 | TRP D | 316 | 105.107 | 8.146 | 18.646 | 1.00 | 18.90 | D |
| ATOM | 6076 | CE3 | TRP D | 316 | 106.498 | 6.917 | 20.193 | 1.00 | 20.19 | D |
| ATOM | 6077 | CD1 | TRP D | 316 | 105.959 | 10.206 | 18.767 | 1.00 | 20.28 | D |
| ATOM | 6078 | NE1 | TRP D | 316 | 104.994 | 9.428 | 18.177 | 1.00 | 19.42 | D |
| ATOM | 6079 | CZ2 | TRP D | 316 | 104.351 | 7.007 | 18.344 | 1.00 | 19.58 | D |
| ATOM | 6080 | CZ3 | TRP D | 316 | 105.741 | 5.778 | 19.892 | 1.00 | 21.16 | D |
| ATOM | 6081 | CH2 | TRP D | 316 | 104.682 | 5.835 | 18.975 | 1.00 | 20.34 | D |
| ATOM | 6082 | C | TRP D | 316 | 108.216 | 10.292 | 23.091 | 1.00 | 27.23 | D |
| ATOM | 6083 | O | TRP D | 316 | 108.854 | 9.311 | 23.519 | 1.00 | 27.64 | D |
| ATOM | 6084 | N | LYS D | 317 | 108.374 | 11.525 | 23.556 | 1.00 | 27.58 | D |
| ATOM | 6085 | CA | LYS D | 317 | 109.332 | 11.847 | 24.602 | 1.00 | 27.23 | D |
| ATOM | 6086 | CS | LYS D | 317 | 109.463 | 13.360 | 24.759 | 1.00 | 29.14 | D |
| ATOM | 6087 | CG | LYS D | 317 | 110.589 | 13.760 | 25.682 | 1.00 | 32.80 | D |
| ATOM | 6088 | CD | LYS D | 317 | 110.743 | 15.264 | 25.749 | 1.00 | 35.26 | D |
| ATOM | 6089 | CE | LYS D | 317 | 111.774 | 15.652 | 26.805 | 1.00 | 37.31 | D |
| ATOM | 6090 | NZ | LYS D | 317 | 111.793 | 17.120 | 27.068 | 1.00 | 37.03 | D |
| ATOM | 6091 | C | LYS D | 317 | 108.822 | 11.275 | 25.910 | 1.00 | 25.94 | D |
| ATOM | 6092 | O | LYS D | 317 | 109.570 | 10.685 | 26.689 | 1.00 | 25.94 | D |
| ATOM | 6093 | N | LYS D | 318 | 107.533 | 11.473 | 26.142 | 1.00 | 23.54 | D |
| ATOM | 6094 | CA | LYS D | 318 | 106.921 | 10.980 | 27.345 | 1.00 | 21.52 | D |
| ATOM | 6095 | CB | LYS D | 318 | 105.457 | 11.403 | 27.426 | 1.00 | 19.84 | D |
| ATOM | 6096 | CG | LYS D | 318 | 105.276 | 12.802 | 27.949 | 1.00 | 15.81 | D |
| ATOM | 6097 | CD | LYS D | 318 | 103.822 | 13.127 | 28.039 | 1.00 | 17.16 | D |
| ATOM | 6098 | CE | LYS D | 318 | 103.592 | 14.588 | 28.439 | 1.00 | 19.94 | D |
| ATOM | 6099 | NZ | LYS D | 318 | 104.446 | 15.524 | 27.676 | 1.00 | 22.18 | D |
| ATOM | 6100 | C | LYS D | 318 | 107.026 | 9.474 | 27.389 | 1.00 | 21.47 | D |
| ATOM | 6101 | O | LYS D | 318 | 107.353 | 8.890 | 28.423 | 1.00 | 22.72 | D |
| ATOM | 6102 | N | LEU D | 319 | 106.754 | 8.825 | 26.271 | 1.00 | 20.41 | D |
| ATOM | 6103 | CA | LEU D | 319 | 106.834 | 7.373 | 26.259 | 1.00 | 19.54 | D |
| ATOM | 6104 | CB | LEU D | 319 | 106.427 | 6.839 | 24.894 | 1.00 | 17.41 | D |
| ATOM | 6105 | CG | LEU D | 319 | 104.933 | 6.610 | 24.750 | 1.00 | 14.60 | D |
| ATOM | 6106 | CD1 | LEU D | 319 | 104.590 | 6.417 | 23.307 | 1.00 | 15.84 | D |
| ATOM | 6107 | CD2 | LEU D | 319 | 104.520 | 5.398 | 25.581 | 1.00 | 13.04 | D |
| ATOM | 6108 | C | LEU D | 319 | 108.232 | 6.901 | 26.605 | 1.00 | 19.33 | D |
| ATOM | 6109 | O | LEU D | 319 | 108.395 | 6.014 | 27.441 | 1.00 | 18.41 | D |
| ATOM | 6110 | N | LEU D | 320 | 109.239 | 7.493 | 25.961 | 1.00 | 20.82 | D |
| ATOM | 6111 | CA | LEU D | 320 | 110.624 | 7.099 | 26.217 | 1.00 | 21.24 | D |
| ATOM | 6112 | CB | LEU D | 320 | 111.589 | 7.926 | 25.344 | 1.00 | 17.94 | D |
| ATOM | 6113 | CG | LEU D | 320 | 111.710 | 7.492 | 23.869 | 1.00 | 18.89 | D |
| ATOM | 6114 | CD1 | LEU D | 320 | 112.467 | 8.533 | 23.030 | 1.00 | 16.82 | D |
| ATOM | 6115 | CD2 | LEU D | 320 | 112.402 | 6.158 | 23.797 | 1.00 | 15.44 | D |
| ATOM | 6116 | C | LEU D | 320 | 110.965 | 7.259 | 27.701 | 1.00 | 22.18 | D |
| ATOM | 6117 | O | LEU D | 320 | 111.574 | 6.383 | 28.310 | 1.00 | 23.23 | D |
| ATOM | 6118 | N | ILE D | 321 | 110.545 | 8.375 | 28.281 | 1.00 | 22.59 | D |
| ATOM | 6119 | CA | ILE D | 321 | 110.824 | 8.661 | 29.676 | 1.00 | 22.13 | D |
| ATOM | 6120 | CB | ILE D | 321 | 110.462 | 10.125 | 30.007 | 1.00 | 21.71 | D |
| ATOM | 6121 | CG2 | ILE D | 321 | 110.406 | 10.336 | 31.509 | 1.00 | 20.64 | D |
| ATOM | 6122 | CG1 | ILE D | 321 | 111.471 | 11.053 | 29.324 | 1.00 | 20.39 | D |
| ATOM | 6123 | CD1 | ILE D | 321 | 111.312 | 12.527 | 29.639 | 1.00 | 17.62 | D |
| ATOM | 6124 | C | ILE D | 321 | 110.112 | 7.729 | 30.641 | 1.00 | 21.87 | D |
| ATOM | 6125 | O | ILE D | 321 | 110.726 | 7.204 | 31.551 | 1.00 | 23.61 | D |
| ATOM | 6126 | N | SER D | 322 | 108.819 | 7.526 | 30.449 | 1.00 | 22.79 | D |
| ATOM | 6127 | CA | SER D | 322 | 108.082 | 6.663 | 31.345 | 1.00 | 23.92 | D |
| ATOM | 6128 | CB | SER D | 322 | 106.610 | 6.624 | 30.943 | 1.00 | 24.36 | D |
| ATOM | 6129 | OG | SER D | 322 | 106.464 | 6.398 | 29.554 | 1.00 | 28.99 | D |
| ATOM | 6130 | C | SER D | 322 | 108.668 | 5.256 | 31.377 | 1.00 | 25.68 | D |
| ATOM | 6131 | O | SER D | 322 | 108.780 | 4.660 | 32.440 | 1.00 | 27.76 | D |
| ATOM | 6132 | N | GLU D | 323 | 109.041 | 4.718 | 30.222 | 1.00 | 24.70 | D |
| ATOM | 6133 | CA | GLU D | 323 | 109.625 | 3.389 | 30.188 | 1.00 | 23.55 | D |
| ATOM | 6134 | CB | GLU D | 323 | 109.581 | 2.835 | 28.775 | 1.00 | 24.33 | D |
| ATOM | 6135 | CG | GLU D | 323 | 108.228 | 2.303 | 28.385 | 1.00 | 25.11 | D |
| ATOM | 6136 | CD | GLU D | 323 | 107.953 | 0.951 | 29.008 | 1.00 | 26.37 | D |
| ATOM | 6137 | OE1 | GLU D | 323 | 108.775 | 0.024 | 28.805 | 1.00 | 26.30 | D |
| ATOM | 6138 | OE2 | GLU D | 323 | 106.920 | 0.809 | 29.695 | 1.00 | 27.36 | D |
| ATOM | 6139 | C | GLU D | 323 | 111.059 | 3.448 | 30.678 | 1.00 | 23.31 | D |
| ATOM | 6140 | O | GLU D | 323 | 111.773 | 2.443 | 30.684 | 1.00 | 23.15 | D |
| ATOM | 6141 | N | ASN D | 324 | 111.467 | 4.644 | 31.091 | 1.00 | 22.23 | D |
| ATOM | 6142 | CA | ASN D | 324 | 112.812 | 4.891 | 31.592 | 1.00 | 22.59 | D |
| ATOM | 6143 | CB | ASN D | 324 | 113.038 | 4.090 | 32.877 | 1.00 | 22.90 | D |
| ATOM | 6144 | CG | ASN D | 324 | 111.864 | 4.200 | 33.858 | 1.00 | 26.71 | D |
| ATOM | 6145 | OD1 | ASN D | 324 | 111.346 | 5.294 | 34.109 | 1.00 | 25.11 | D |
| ATOM | 6146 | ND2 | ASN D | 324 | 111.450 | 3.056 | 34.430 | 1.00 | 28.34 | D |
| ATOM | 6147 | C | ASN D | 324 | 113.911 | 4.565 | 30.560 | 1.00 | 23.12 | D |
| ATOM | 6148 | O | ASN D | 324 | 114.971 | 4.031 | 30.914 | 1.00 | 23.08 | D |
| ATOM | 6149 | N | PHE D | 325 | 113.667 | 4.881 | 29.285 | 1.00 | 22.49 | D |
| ATOM | 6150 | CA | PHE D | 325 | 114.673 | 4.613 | 28.246 | 1.00 | 22.20 | D |
| ATOM | 6151 | CB | PHE D | 325 | 114.005 | 4.332 | 26.882 | 1.00 | 18.54 | D |
| ATOM | 6152 | CG | PHE D | 325 | 113.296 | 2.991 | 26.806 | 1.00 | 18.28 | D |
| ATOM | 6153 | CD1 | PHE D | 325 | 112.080 | 2.864 | 26.117 | 1.00 | 17.36 | D |
| ATOM | 6154 | CD2 | PHE D | 325 | 113.828 | 1.860 | 27.425 | 1.00 | 17.01 | D |
| ATOM | 6155 | CE1 | PHE D | 325 | 111.404 | 1.631 | 26.053 | 1.00 | 15.65 | D |
| ATOM | 6156 | CE2 | PHE D | 325 | 113.165 | 0.617 | 27.369 | 1.00 | 16.41 | D |
| ATOM | 6157 | CZ | PHE D | 325 | 111.942 | 0.508 | 26.677 | 1.00 | 16.67 | D |
| ATOM | 6158 | C | PHE D | 325 | 115.640 | 5.798 | 28.136 | 1.00 | 21.95 | D |
| ATOM | 6159 | O | PHE D | 325 | 116.735 | 5.686 | 27.578 | 1.00 | 21.44 | D |
| ATOM | 6160 | N | VAL D | 326 | 115.240 | 6.925 | 28.707 | 1.00 | 21.18 | D |
| ATOM | 6161 | CA | VAL D | 326 | 116.059 | 8.113 | 28.642 | 1.00 | 21.85 | D |
| ATOM | 6162 | CB | VAL D | 326 | 115.922 | 8.765 | 27.238 | 1.00 | 21.90 | D |
| ATOM | 6163 | CG1 | VAL D | 326 | 114.455 | 9.085 | 26.972 | 1.00 | 22.16 | D |
| ATOM | 6164 | CG2 | VAL D | 326 | 116.772 | 10.016 | 27.133 | 1.00 | 19.90 | D |
| ATOM | 6165 | C | VAL D | 326 | 115.619 | 9.084 | 29.725 | 1.00 | 22.99 | D |
| ATOM | 6166 | O | VAL D | 326 | 114.453 | 9.105 | 30.121 | 1.00 | 23.26 | D |
| ATOM | 6167 | N | SER D | 327 | 116.571 | 9.869 | 30.215 | 1.00 | 26.57 | D |
| ATOM | 6168 | CA | SER D | 327 | 116.308 | 10.853 | 31.257 | 1.00 | 30.18 | D |
| ATOM | 6169 | CB | SER D | 327 | 117.552 | 11.111 | 32.099 | 1.00 | 28.88 | D |
| ATOM | 6170 | OG | SER D | 327 | 117.794 | 10.061 | 33.008 | 1.00 | 34.20 | D |
| ATOM | 6171 | C | SER D | 327 | 115.914 | 12.167 | 30.648 | 1.00 | 32.93 | D |
| ATOM | 6172 | O | SER D | 327 | 116.304 | 12.488 | 29.533 | 1.00 | 33.50 | D |
| ATOM | 6173 | N | PRO D | 328 | 115.141 | 12.958 | 31.384 | 1.00 | 36.30 | D |
| ATOM | 6174 | CD | PRO D | 328 | 114.483 | 12.591 | 32.644 | 1.00 | 38.28 | D |
| ATOM | 6175 | CA | PRO D | 328 | 114.693 | 14.269 | 30.923 | 1.00 | 38.59 | D |
| ATOM | 6176 | CB | PRO D | 328 | 113.988 | 14.834 | 32.140 | 1.00 | 38.19 | D |
| ATOM | 6177 | CG | PRO D | 328 | 113.360 | 13.609 | 32.728 | 1.00 | 39.16 | D |
| ATOM | 6178 | C | PRO D | 328 | 115.900 | 15.110 | 30.526 | 1.00 | 40.86 | D |
| ATOM | 6179 | O | PRO D | 328 | 115.886 | 15.787 | 29.496 | 1.00 | 42.18 | D |
| ATOM | 6180 | N | LYS D | 329 | 116.955 | 15.053 | 31.332 | 1.00 | 41.28 | D |
| ATOM | 6181 | CA | LYS D | 329 | 118.143 | 15.826 | 31.027 | 1.00 | 42.86 | D |
| ATOM | 6182 | CB | LYS D | 329 | 118.861 | 16.228 | 32.327 | 1.00 | 46.62 | D |
| ATOM | 6183 | CG | LYS D | 329 | 118.066 | 17.272 | 33.169 | 1.00 | 49.10 | D |
| ATOM | 6184 | CD | LYS D | 329 | 118.615 | 17.445 | 34.594 | 1.00 | 50.67 | D |
| ATOM | 6185 | CE | LYS D | 329 | 120.077 | 17.887 | 34.606 | 1.00 | 51.76 | D |
| ATOM | 6186 | NZ | LYS D | 329 | 120.645 | 17.988 | 35.993 | 1.00 | 51.50 | D |
| ATOM | 6187 | C | LYS D | 329 | 119.078 | 15.102 | 30.073 | 1.00 | 42.35 | D |
| ATOM | 6188 | O | LYS D | 329 | 120.060 | 15.671 | 29.609 | 1.00 | 43.19 | D |
| ATOM | 6189 | N | GLY D | 330 | 118.766 | 13.850 | 29.765 | 1.00 | 41.86 | D |
| ATOM | 6190 | CA | GLY D | 330 | 119.597 | 13.107 | 28.837 | 1.00 | 40.87 | D |
| ATOM | 6191 | C | GLY D | 330 | 118.961 | 13.089 | 27.456 | 1.00 | 42.32 | D |
| ATOM | 6192 | O | GLY D | 330 | 119.470 | 12.461 | 26.528 | 1.00 | 41.14 | D |
| ATOM | 6193 | N | PHE D | 331 | 117.849 | 13.798 | 27.304 | 1.00 | 44.29 | D |
| ATOM | 6194 | CA | PHE D | 331 | 117.148 | 13.809 | 26.026 | 1.00 | 46.40 | D |
| ATOM | 6195 | CB | PHE D | 331 | 115.887 | 14.660 | 26.119 | 1.00 | 47.33 | D |
| ATOM | 6196 | CG | PHE D | 331 | 114.896 | 14.396 | 25.016 | 1.00 | 49.12 | D |
| ATOM | 6197 | CD1 | PHE D | 331 | 114.198 | 13.188 | 24.958 | 1.00 | 48.42 | D |
| ATOM | 6198 | CD2 | PHE D | 331 | 114.658 | 15.359 | 24.033 | 1.00 | 49.78 | D |
| ATOM | 6199 | CE1 | PHE D | 331 | 113.273 | 12.942 | 23.939 | 1.00 | 48.28 | D |
| ATOM | 6200 | CE2 | PHE D | 331 | 113.735 | 15.128 | 23.008 | 1.00 | 49.07 | D |
| ATOM | 6201 | CZ | PHE D | 331 | 113.042 | 13.917 | 22.958 | 1.00 | 49.05 | D |
| ATOM | 6202 | C | PHE D | 331 | 117.983 | 14.276 | 24.846 | 1.00 | 46.95 | D |
| ATOM | 6203 | O | PHE D | 331 | 118.017 | 13.613 | 23.815 | 1.00 | 48.73 | D |
| ATOM | 6204 | N | ASN D | 332 | 118.651 | 15.414 | 24.997 | 1.00 | 47.11 | D |
| ATOM | 6205 | CA | ASN D | 332 | 119.477 | 15.972 | 23.932 | 1.00 | 46.02 | D |
| ATOM | 6206 | CB | ASN D | 332 | 120.255 | 17.161 | 24.463 | 1.00 | 47.82 | D |
| ATOM | 6207 | CG | ASN D | 332 | 119.497 | 18.440 | 24.335 | 1.00 | 49.50 | D |
| ATOM | 6208 | OD1 | ASN D | 332 | 118.322 | 18.519 | 24.692 | 1.00 | 50.47 | D |
| ATOM | 6209 | ND2 | ASN D | 332 | 120.165 | 19.468 | 23.815 | 1.00 | 52.34 | D |
| ATOM | 6210 | C | ASN D | 332 | 120.451 | 14.972 | 23.336 | 1.00 | 45.93 | D |
| ATOM | 6211 | O | ASN D | 332 | 120.420 | 14.681 | 22.140 | 1.00 | 45.54 | D |
| ATOM | 6212 | N | SER D | 333 | 121.326 | 14.443 | 24.175 | 1.00 | 46.43 | D |
| ATOM | 6213 | CA | SER D | 333 | 122.308 | 13.487 | 23.702 | 1.00 | 45.98 | D |
| ATOM | 6214 | CB | SER D | 333 | 123.043 | 12.863 | 24.885 | 1.00 | 46.48 | D |
| ATOM | 6215 | OG | SER D | 333 | 123.853 | 11.783 | 24.454 | 1.00 | 49.00 | D |
| ATOM | 6216 | C | SER D | 333 | 121.659 | 12.392 | 22.851 | 1.00 | 44.33 | D |
| ATOM | 6217 | O | SER D | 333 | 122.129 | 12.099 | 21.751 | 1.00 | 44.40 | D |
| ATOM | 6218 | N | LED D | 334 | 120.577 | 11.795 | 23.344 | 1.00 | 42.68 | D |
| ATOM | 6219 | CA | LED D | 334 | 119.912 | 10.728 | 22.591 | 1.00 | 41.52 | D |
| ATOM | 6220 | CB | LED D | 334 | 118.777 | 10.098 | 23.407 | 1.00 | 39.72 | D |
| ATOM | 6221 | CG | LED D | 334 | 117.873 | 9.152 | 22.604 | 1.00 | 38.51 | D |
| ATOM | 6222 | CD1 | LED D | 334 | 118.687 | 7.997 | 22.053 | 1.00 | 37.25 | D |
| ATOM | 6223 | CD2 | LED D | 334 | 116.746 | 8.651 | 23.475 | 1.00 | 37.57 | D |
| ATOM | 6224 | C | LED D | 334 | 119.352 | 11.228 | 21.268 | 1.00 | 39.88 | D |
| ATOM | 6225 | O | LEU D | 334 | 119.439 | 10.553 | 20.242 | 1.00 | 37.95 | D |
| ATOM | 6226 | N | ASN D | 335 | 118.758 | 12.410 | 21.304 | 1.00 | 39.39 | D |
| ATOM | 6227 | CA | ASN D | 335 | 118.202 | 12.973 | 20.096 | 1.00 | 39.71 | D |
| ATOM | 6228 | CB | ASN D | 335 | 117.463 | 14.263 | 20.399 | 1.00 | 40.92 | D |
| ATOM | 6229 | CG | ASN D | 335 | 116.080 | 14.255 | 19.829 | 1.00 | 42.75 | D |
| ATOM | 6230 | OD1 | ASN D | 335 | 115.879 | 13.877 | 18.676 | 1.00 | 43.99 | D |
| ATOM | 6231 | ND2 | ASN D | 335 | 115.108 | 14.669 | 20.630 | 1.00 | 44.96 | D |
| ATOM | 6232 | C | ASN D | 335 | 119.299 | 13.229 | 19.066 | 1.00 | 38.88 | D |
| ATOM | 6233 | O | ASN D | 335 | 119.209 | 12.772 | 17.926 | 1.00 | 39.28 | D |
| ATOM | 6234 | N | LED D | 336 | 120.341 | 13.945 | 19.469 | 1.00 | 36.54 | D |
| ATOM | 6235 | CA | LED D | 336 | 121.426 | 14.216 | 18.555 | 1.00 | 35.78 | D |
| ATOM | 6236 | CB | LED D | 336 | 122.488 | 15.081 | 19.230 | 1.00 | 35.28 | D |
| ATOM | 6237 | CG | LED D | 336 | 123.704 | 15.571 | 18.421 | 1.00 | 35.06 | D |
| ATOM | 6238 | CD1 | LED D | 336 | 124.755 | 14.482 | 18.364 | 1.00 | 33.88 | D |
| ATOM | 6239 | CD2 | LED D | 336 | 123.284 | 16.002 | 17.027 | 1.00 | 33.93 | D |
| ATOM | 6240 | C | LED D | 336 | 122.021 | 12.905 | 18.034 | 1.00 | 36.03 | D |
| ATOM | 6241 | O | LED D | 336 | 122.542 | 12.857 | 16.920 | 1.00 | 37.69 | D |
| ATOM | 6242 | N | LYS D | 337 | 121.919 | 11.833 | 18.812 | 1.00 | 36.19 | D |
| ATOM | 6243 | CA | LYS D | 337 | 122.439 | 10.529 | 18.378 | 1.00 | 37.20 | D |
| ATOM | 6244 | CB | LYS D | 337 | 122.452 | 9.533 | 19.547 | 1.00 | 38.40 | D |
| ATOM | 6245 | CG | LYS D | 337 | 123.706 | 9.512 | 20.409 | 1.00 | 40.10 | D |
| ATOM | 6246 | CD | LYS D | 337 | 124.743 | 8.543 | 19.848 | 1.00 | 41.01 | D |
| ATOM | 6247 | CE | LYS D | 337 | 125.896 | 8.283 | 20.827 | 1.00 | 41.41 | D |
| ATOM | 6248 | NZ | LYS D | 337 | 125.504 | 7.528 | 22.071 | 1.00 | 40.33 | D |
| ATOM | 6249 | C | LYS D | 337 | 121.576 | 9.939 | 17.257 | 1.00 | 37.31 | D |
| ATOM | 6250 | O | LYS D | 337 | 122.081 | 9.279 | 16.344 | 1.00 | 37.48 | D |
| ATOM | 6251 | N | LED D | 338 | 120.268 | 10.164 | 17.347 | 1.00 | 36.91 | D |
| ATOM | 6252 | CA | LED D | 338 | 119.332 | 9.654 | 16.351 | 1.00 | 36.87 | D |
| ATOM | 6253 | CB | LED D | 338 | 117.893 | 9.797 | 16.863 | 1.00 | 35.09 | D |
| ATOM | 6254 | CG | LED D | 338 | 117.529 | 8.902 | 18.052 | 1.00 | 33.33 | D |
| ATOM | 6255 | CD1 | LED D | 338 | 116.175 | 9.300 | 18.621 | 1.00 | 31.89 | D |
| ATOM | 6256 | CD2 | LED D | 338 | 117.533 | 7.450 | 17.600 | 1.00 | 31.70 | D |
| ATOM | 6257 | C | LED D | 338 | 119.513 | 10.412 | 15.044 | 1.00 | 37.22 | D |
| ATOM | 6258 | O | LED D | 338 | 119.418 | 9.841 | 13.968 | 1.00 | 36.68 | D |
| ATOM | 6259 | N | SER D | 339 | 119.791 | 11.703 | 15.161 | 1.00 | 37.23 | D |
| ATOM | 6260 | CA | SER D | 339 | 120.000 | 12.537 | 14.006 | 1.00 | 35.92 | D |
| ATOM | 6261 | CB | SER D | 339 | 120.390 | 13.933 | 14.426 | 1.00 | 34.24 | D |
| ATOM | 6262 | OG | SER D | 339 | 120.457 | 14.756 | 13.283 | 1.00 | 37.68 | D |
| ATOM | 6263 | C | SER D | 339 | 121.094 | 11.973 | 13.124 | 1.00 | 36.83 | D |
| ATOM | 6264 | O | SER D | 339 | 120.978 | 12.005 | 11.908 | 1.00 | 36.94 | D |
| ATOM | 6265 | N | GLN D | 340 | 122.161 | 11.467 | 13.735 | 1.00 | 36.98 | D |
| ATOM | 6266 | CA | GLN D | 340 | 123.266 | 10.895 | 12.975 | 1.00 | 37.54 | D |
| ATOM | 6267 | CB | GLN D | 340 | 124.443 | 10.581 | 13.883 | 1.00 | 38.76 | D |
| ATOM | 6268 | CG | GLN D | 340 | 124.489 | 11.408 | 15.144 | 1.00 | 42.76 | D |
| ATOM | 6269 | CD | GLN D | 340 | 125.519 | 12.491 | 15.095 | 1.00 | 43.21 | D |
| ATOM | 6270 | OE1 | GLN D | 340 | 125.816 | 13.113 | 16.105 | 1.00 | 43.96 | D |
| ATOM | 6271 | NE2 | GLN D | 340 | 126.077 | 12.727 | 13.917 | 1.00 | 47.75 | D |
| ATOM | 6272 | C | GLN D | 340 | 122.786 | 9.592 | 12.376 | 1.00 | 38.02 | D |
| ATOM | 6273 | O | GLN D | 340 | 123.099 | 9.254 | 11.235 | 1.00 | 36.84 | D |
| ATOM | 6274 | N | LYS D | 341 | 122.023 | 8.857 | 13.172 | 1.00 | 38.91 | D |
| ATOM | 6275 | CA | LYS D | 341 | 121.503 | 7.564 | 12.759 | 1.00 | 40.04 | D |
| ATOM | 6276 | CB | LYS D | 341 | 120.908 | 6.842 | 13.979 | 1.00 | 41.15 | D |
| ATOM | 6277 | CG | LYS D | 341 | 120.440 | 5.417 | 13.722 | 1.00 | 43.83 | D |
| ATOM | 6278 | CD | LYS D | 341 | 121.542 | 4.580 | 13.081 | 1.00 | 46.84 | D |
| ATOM | 6279 | CE | LYS D | 341 | 121.018 | 3.233 | 12.559 | 1.00 | 48.14 | D |
| ATOM | 6280 | NZ | LYS D | 341 | 122.101 | 2.428 | 11.901 | 1.00 | 48.02 | D |
| ATOM | 6281 | C | LYS D | 341 | 120.463 | 7.656 | 11.641 | 1.00 | 40.03 | D |
| ATOM | 6282 | O | LYS D | 341 | 120.548 | 6.935 | 10.647 | 1.00 | 40.71 | D |
| ATOM | 6283 | N | TYR D | 342 | 119.494 | 8.551 | 11.804 | 1.00 | 39.52 | D |
| ATOM | 6284 | CA | TYR D | 342 | 118.413 | 8.726 | 10.840 | 1.00 | 39.22 | D |
| ATOM | 6285 | CB | TYR D | 342 | 117.084 | 8.349 | 11.504 | 1.00 | 40.25 | D |
| ATOM | 6286 | CG | TYR D | 342 | 117.093 | 6.975 | 12.120 | 1.00 | 41.96 | D |
| ATOM | 6287 | CD1 | TYR D | 342 | 117.151 | 5.839 | 11.322 | 1.00 | 44.58 | D |
| ATOM | 6288 | CE1 | TYR D | 342 | 117.195 | 4.559 | 11.887 | 1.00 | 46.16 | D |
| ATOM | 6289 | CD2 | TYR D | 342 | 117.076 | 6.805 | 13.501 | 1.00 | 43.49 | D |
| ATOM | 6290 | CE2 | TYR D | 342 | 117.119 | 5.530 | 14.078 | 1.00 | 44.33 | D |
| ATOM | 6291 | CZ | TYR D | 342 | 117.180 | 4.412 | 13.263 | 1.00 | 45.41 | D |
| ATOM | 6292 | OH | TYR D | 342 | 117.244 | 3.145 | 13.796 | 1.00 | 44.64 | D |
| ATOM | 6293 | C | TYR D | 342 | 118.315 | 10.155 | 10.287 | 1.00 | 38.30 | D |
| ATOM | 6294 | O | TYR D | 342 | 117.437 | 10.926 | 10.687 | 1.00 | 38.68 | D |
| ATOM | 6295 | N | PRO D | 343 | 119.207 | 10.522 | 9.354 | 1.00 | 36.69 | D |
| ATOM | 6296 | CD | PRO D | 343 | 120.303 | 9.690 | 8.831 | 1.00 | 33.93 | D |
| ATOM | 6297 | CA | PRO D | 343 | 119.222 | 11.859 | 8.744 | 1.00 | 35.64 | D |
| ATOM | 6298 | CB | PRO D | 343 | 120.322 | 11.747 | 7.701 | 1.00 | 33.94 | D |
| ATOM | 6299 | CG | PRO D | 343 | 121.227 | 10.708 | 8.264 | 1.00 | 33.00 | D |
| ATOM | 6300 | C | PRO D | 343 | 117.897 | 12.243 | 8.100 | 1.00 | 36.66 | D |
| ATOM | 6301 | O | PRO D | 343 | 117.387 | 13.353 | 8.298 | 1.00 | 36.68 | D |
| ATOM | 6302 | N | LYS D | 344 | 117.345 | 11.306 | 7.333 | 1.00 | 37.98 | D |
| ATOM | 6303 | CA | LYS D | 344 | 116.097 | 11.521 | 6.600 | 1.00 | 38.87 | D |
| ATOM | 6304 | CB | LYS D | 344 | 116.021 | 10.554 | 5.402 | 1.00 | 40.52 | D |
| ATOM | 6305 | CG | LYS D | 344 | 116.552 | 9.140 | 5.658 | 1.00 | 43.94 | D |
| ATOM | 6306 | CD | LYS D | 344 | 115.598 | 8.274 | 6.494 | 1.00 | 44.39 | D |
| ATOM | 6307 | CE | LYS D | 344 | 116.370 | 7.166 | 7.227 | 1.00 | 43.37 | D |
| ATOM | 6308 | NZ | LYS D | 344 | 117.439 | 7.751 | 8.094 | 1.00 | 37.93 | D |
| ATOM | 6309 | C | LYS D | 344 | 114.791 | 11.471 | 7.396 | 1.00 | 37.24 | D |
| ATOM | 6310 | O | LYS D | 344 | 113.779 | 12.011 | 6.947 | 1.00 | 38.60 | D |
| ATOM | 6311 | N | LEU D | 345 | 114.808 | 10.851 | 8.572 | 1.00 | 34.93 | D |
| ATOM | 6312 | CA | LEU D | 345 | 113.597 | 10.766 | 9.382 | 1.00 | 32.64 | D |
| ATOM | 6313 | CB | LEU D | 345 | 113.670 | 9.573 | 10.329 | 1.00 | 31.24 | D |
| ATOM | 6314 | CG | LEU D | 345 | 113.726 | 8.216 | 9.628 | 1.00 | 29.39 | D |
| ATOM | 6315 | CD1 | LEU D | 345 | 113.660 | 7.118 | 10.667 | 1.00 | 30.86 | D |
| ATOM | 6316 | CD2 | LEU D | 345 | 112.560 | 8.084 | 8.666 | 1.00 | 30.92 | D |
| ATOM | 6317 | C | LEU D | 345 | 113.376 | 12.033 | 10.176 | 1.00 | 31.59 | D |
| ATOM | 6318 | O | LEU D | 345 | 114.318 | 12.780 | 10.422 | 1.00 | 32.92 | D |
| ATOM | 6319 | N | SER D | 346 | 112.130 | 12.298 | 10.557 | 1.00 | 30.59 | D |
| ATOM | 6320 | CA | SER D | 346 | 111.836 | 13.500 | 11.338 | 1.00 | 29.97 | D |
| ATOM | 6321 | CB | SER D | 346 | 110.377 | 13.931 | 11.158 | 1.00 | 29.48 | D |
| ATOM | 6322 | OG | SER D | 346 | 109.498 | 12.856 | 11.422 | 1.00 | 30.59 | D |
| ATOM | 6323 | C | SER D | 346 | 112.101 | 13.212 | 12.800 | 1.00 | 29.74 | D |
| ATOM | 6324 | O | SER D | 346 | 112.387 | 12.069 | 13.184 | 1.00 | 28.91 | D |
| ATOM | 6325 | N | GLN D | 347 | 111.992 | 14.233 | 13.634 | 1.00 | 28.38 | D |
| ATOM | 6326 | CA | GLN D | 347 | 112.255 | 13.991 | 15.036 | 1.00 | 28.40 | D |
| ATOM | 6327 | CB | GLN D | 347 | 112.237 | 15.291 | 15.818 | 1.00 | 29.66 | D |
| ATOM | 6328 | CG | GLN D | 347 | 112.819 | 15.144 | 17.190 | 1.00 | 32.02 | D |
| ATOM | 6329 | CD | GLN D | 347 | 112.905 | 16.474 | 17.879 | 1.00 | 35.29 | D |
| ATOM | 6330 | OE1 | GLN D | 347 | 111.889 | 17.161 | 18.035 | 1.00 | 38.15 | D |
| ATOM | 6331 | NE2 | GLN D | 347 | 114.111 | 16.861 | 18.292 | 1.00 | 33.64 | D |
| ATOM | 6332 | C | GLN D | 347 | 111.277 | 13.007 | 15.663 | 1.00 | 26.76 | D |
| ATOM | 6333 | O | GLN D | 347 | 111.694 | 12.085 | 16.357 | 1.00 | 26.57 | D |
| ATOM | 6334 | N | GLN D | 348 | 109.984 | 13.203 | 15.427 | 1.00 | 25.38 | D |
| ATOM | 6335 | CA | GLN D | 348 | 108.996 | 12.306 | 16.001 | 1.00 | 24.96 | D |
| ATOM | 6336 | CS | GLN D | 348 | 107.581 | 12.739 | 15.657 | 1.00 | 24.21 | D |
| ATOM | 6337 | CG | GLN D | 348 | 107.267 | 14.152 | 16.034 | 1.00 | 24.28 | D |
| ATOM | 6338 | CD | GLN D | 348 | 107.648 | 15.126 | 14.947 | 1.00 | 23.84 | D |
| ATOM | 6339 | OE1 | GLN D | 348 | 108.598 | 14.899 | 14.211 | 1.00 | 24.03 | D |
| ATOM | 6340 | NE2 | GLN D | 348 | 106.908 | 16.224 | 14.845 | 1.00 | 24.64 | D |
| ATOM | 6341 | C | GLN D | 348 | 109.209 | 10.896 | 15.483 | 1.00 | 26.54 | D |
| ATOM | 6342 | O | GLN D | 348 | 109.118 | 9.921 | 16.233 | 1.00 | 27.22 | D |
| ATOM | 6343 | N | ASP D | 349 | 109.499 | 10.791 | 14.193 | 1.00 | 26.70 | D |
| ATOM | 6344 | CA | ASP D | 349 | 109.718 | 9.500 | 13.588 | 1.00 | 26.16 | D |
| ATOM | 6345 | CB | ASP D | 349 | 109.918 | 9.666 | 12.086 | 1.00 | 30.05 | D |
| ATOM | 6346 | CG | ASP D | 349 | 108.666 | 9.338 | 11.295 | 1.00 | 33.76 | D |
| ATOM | 6347 | OD1 | ASP D | 349 | 107.598 | 9.889 | 11.612 | 1.00 | 35.36 | D |
| ATOM | 6348 | OD2 | ASP D | 349 | 108.754 | 8.521 | 10.354 | 1.00 | 37.16 | D |
| ATOM | 6349 | C | ASP D | 349 | 110.900 | 8.775 | 14.220 | 1.00 | 25.17 | D |
| ATOM | 6350 | O | ASP D | 349 | 110.815 | 7.578 | 14.508 | 1.00 | 25.67 | D |
| ATOM | 6351 | N | ARG D | 350 | 111.999 | 9.491 | 14.430 | 1.00 | 23.55 | D |
| ATOM | 6352 | CA | ARG D | 350 | 113.194 | 8.906 | 15.047 | 1.00 | 23.54 | D |
| ATOM | 6353 | CS | ARG D | 350 | 114.265 | 9.981 | 15.252 | 1.00 | 25.20 | D |
| ATOM | 6354 | CG | ARG D | 350 | 114.885 | 10.512 | 13.994 | 1.00 | 25.82 | D |
| ATOM | 6355 | CD | ARG D | 350 | 115.595 | 11.837 | 14.220 | 1.00 | 25.92 | D |
| ATOM | 6356 | NE | ARG D | 350 | 116.255 | 12.230 | 12.977 | 1.00 | 28.94 | D |
| ATOM | 6357 | CZ | ARG D | 350 | 116.707 | 13.448 | 12.698 | 1.00 | 29.47 | D |
| ATOM | 6358 | NH1 | ARG D | 350 | 116.590 | 14.440 | 13.568 | 1.00 | 32.83 | D |
| ATOM | 6359 | NH2 | ARG D | 350 | 117.281 | 13.673 | 11.534 | 1.00 | 31.17 | D |
| ATOM | 6360 | C | ARG D | 350 | 112.831 | 8.343 | 16.416 | 1.00 | 23.18 | D |
| ATOM | 6361 | O | ARG D | 350 | 112.968 | 7.148 | 16.679 | 1.00 | 22.24 | D |
| ATOM | 6362 | N | LEU D | 351 | 112.376 | 9.242 | 17.282 | 1.00 | 21.94 | D |
| ATOM | 6363 | CA | LEU D | 351 | 111.991 | 8.890 | 18.627 | 1.00 | 20.55 | D |
| ATOM | 6364 | CB | LEU D | 351 | 111.307 | 10.082 | 19.297 | 1.00 | 18.30 | D |
| ATOM | 6365 | CG | LEU D | 351 | 112.227 | 11.278 | 19.543 | 1.00 | 15.70 | D |
| ATOM | 6366 | CD1 | LEU D | 351 | 111.401 | 12.443 | 19.998 | 1.00 | 14.97 | D |
| ATOM | 6367 | CD2 | LEU D | 351 | 113.315 | 10.927 | 20.561 | 1.00 | 12.36 | D |
| ATOM | 6368 | C | LEU D | 351 | 111.081 | 7.671 | 18.627 | 1.00 | 20.79 | D |
| ATOM | 6369 | O | LEU D | 351 | 111.175 | 6.824 | 19.508 | 1.00 | 21.85 | D |
| ATOM | 6370 | N | ARG D | 352 | 110.211 | 7.569 | 17.631 | 1.00 | 21.27 | D |
| ATOM | 6371 | CA | ARG D | 352 | 109.333 | 6.421 | 17.566 | 1.00 | 22.32 | D |
| ATOM | 6372 | CB | ARG D | 352 | 108.251 | 6.616 | 16.517 | 1.00 | 23.50 | D |
| ATOM | 6373 | CG | ARG D | 352 | 107.278 | 5.440 | 16.398 | 1.00 | 25.41 | D |
| ATOM | 6374 | CD | ARG D | 352 | 106.075 | 5.874 | 15.568 | 1.00 | 26.94 | D |
| ATOM | 6375 | NE | ARG D | 352 | 106.491 | 6.312 | 14.248 | 1.00 | 27.67 | D |
| ATOM | 6376 | CZ | ARG D | 352 | 106.345 | 5.590 | 13.151 | 1.00 | 27.98 | D |
| ATOM | 6377 | NH1 | ARG D | 352 | 105.772 | 4.384 | 13.223 | 1.00 | 28.77 | D |
| ATOM | 6378 | NH2 | ARG D | 352 | 106.801 | 6.071 | 11.997 | 1.00 | 28.35 | D |
| ATOM | 6379 | C | ARG D | 352 | 110.132 | 5.156 | 17.257 | 1.00 | 22.65 | D |
| ATOM | 6380 | O | ARG D | 352 | 109.852 | 4.088 | 17.809 | 1.00 | 22.75 | D |
| ATOM | 6381 | N | LEU D | 353 | 111.129 | 5.236 | 16.389 | 1.00 | 23.22 | D |
| ATOM | 6382 | CA | LEU D | 353 | 111.877 | 4.014 | 16.139 | 1.00 | 25.64 | D |
| ATOM | 6383 | CB | LEUD D | 353 | 112.853 | 4.180 | 14.974 | 1.00 | 27.60 | D |
| ATOM | 6384 | CG | LEU D | 353 | 112.211 | 4.675 | 13.687 | 1.00 | 31.53 | D |
| ATOM | 6385 | CD1 | LEU D | 353 | 113.257 | 4.543 | 12.590 | 1.00 | 34.73 | D |
| ATOM | 6386 | CD2 | LEU D | 353 | 110.948 | 3.847 | 13.321 | 1.00 | 34.02 | D |
| ATOM | 6387 | C | LEU D | 353 | 112.640 | 3.650 | 17.408 | 1.00 | 24.34 | D |
| ATOM | 6388 | O | LEU D | 353 | 112.766 | 2.477 | 17.748 | 1.00 | 24.26 | D |
| ATOM | 6389 | N | SER D | 354 | 113.135 | 4.654 | 18.124 | 1.00 | 24.48 | D |
| ATOM | 6390 | CA | SER D | 354 | 113.888 | 4.379 | 19.339 | 1.00 | 24.64 | D |
| ATOM | 6391 | CB | SER D | 354 | 114.399 | 5.670 | 19.953 | 1.00 | 24.29 | O |
| ATOM | 6392 | OG | SER D | 354 | 115.774 | 5.509 | 20.223 | 1.00 | 27.22 | O |
| ATOM | 6393 | C | SER D | 354 | 113.045 | 3.623 | 20.345 | 1.00 | 23.77 | D |
| ATOM | 6394 | O | SER D | 354 | 113.439 | 2.566 | 20.828 | 1.00 | 22.40 | D |
| ATOM | 6395 | N | PHE D | 355 | 111.879 | 4.169 | 20.646 | 1.00 | 23.57 | D |
| ATOM | 6396 | CA | PHE D | 355 | 110.979 | 3.533 | 21.573 | 1.00 | 25.63 | D |
| ATOM | 6397 | CB | PHE D | 355 | 109.660 | 4.283 | 21.596 | 1.00 | 26.61 | D |
| ATOM | 6398 | CG | PHE D | 355 | 108.725 | 3.795 | 22.636 | 1.00 | 27.78 | D |
| ATOM | 6399 | CD1 | PHE D | 355 | 109.055 | 3.919 | 23.978 | 1.00 | 29.88 | D |
| ATOM | 6400 | CD2 | PHE D | 355 | 107.526 | 3.189 | 22.286 | 1.00 | 29.67 | D |
| ATOM | 6401 | CE1 | PHE D | 355 | 108.205 | 3.443 | 24.972 | 1.00 | 29.34 | D |
| ATOM | 6402 | CE2 | PHE D | 355 | 106.664 | 2.707 | 23.269 | 1.00 | 30.25 | D |
| ATOM | 6403 | CZ | PHE D | 355 | 107.011 | 2.836 | 24.614 | 1.00 | 29.64 | D |
| ATOM | 6404 | C | PHE D | 355 | 110.731 | 2.089 | 21.156 | 1.00 | 26.53 | D |
| ATOM | 6405 | O | PHE D | 355 | 110.970 | 1.156 | 21.920 | 1.00 | 26.93 | D |
| ATOM | 6406 | N | LEU D | 356 | 110.257 | 1.904 | 19.931 | 1.00 | 27.97 | D |
| ATOM | 6407 | CA | LEU D | 356 | 109.975 | 0.557 | 19.441 | 1.00 | 29.40 | D |
| ATOM | 6408 | CB | LEU D | 356 | 109.502 | 0.601 | 17.990 | 1.00 | 29.41 | D |
| ATOM | 6409 | CG | LEU D | 356 | 108.233 | 1.408 | 17.701 | 1.00 | 30.81 | D |
| ATOM | 6410 | CD1 | LEU D | 356 | 108.091 | 1.510 | 16.193 | 1.00 | 30.03 | D |
| ATOM | 6411 | CD2 | LEU D | 356 | 107.001 | 0.766 | 18.334 | 1.00 | 27.79 | D |
| ATOM | 6412 | C | LEU D | 35 | 111.164 | −0.387 | 19.557 | 1.00 | 29.19 | D |
| ATOM | 6413 | O | LEU D | 356 | 111.011 | −1.540 | 19.954 | 1.00 | 29.03 | D |
| ATOM | 6414 | N | GLU D | 357 | 112.348 | 0.106 | 19.223 | 1.00 | 29.15 | D |
| ATOM | 6415 | CA | GLU D | 357 | 113.525 | −0.723 | 19.296 | 1.00 | 30.63 | D |
| ATOM | 6416 | CB | GLU D | 357 | 114.677 | −0.053 | 18.560 | 1.00 | 32.16 | D |
| ATOM | 6417 | CG | GLU D | 357 | 115.697 | −1.025 | 18.007 | 1.00 | 37.30 | D |
| ATOM | 6418 | CD | GLU D | 357 | 117.086 | −0.418 | 17.930 | 1.00 | 40.82 | D |
| ATOM | 6419 | OE1 | GLU D | 357 | 117.248 | 0.639 | 17.262 | 1.00 | 41.37 | D |
| ATOM | 6420 | OE2 | GLU D | 357 | 118.017 | −1.000 | 18.548 | 1.00 | 41.48 | D |
| ATOM | 6421 | C | GLU D | 357 | 113.946 | −1.025 | 20.741 | 1.00 | 32.26 | D |
| ATOM | 6422 | O | GLU D | 357 | 114.599 | −2.041 | 21.016 | 1.00 | 31.90 | D |
| ATOM | 6423 | N | ASN D | 358 | 113.571 | −0.148 | 21.665 | 1.00 | 29.36 | D |
| ATOM | 6424 | CA | ASN D | 358 | 113.939 | −0.341 | 23.046 | 1.00 | 27.42 | D |
| ATOM | 6425 | CB | ASN D | 358 | 114.060 | 1.000 | 23.750 | 1.00 | 27.90 | D |
| ATOM | 6426 | CG | ASN D | 358 | 115.443 | 1.598 | 23.610 | 1.00 | 28.81 | D |
| ATOM | 6427 | OD1 | ASN D | 358 | 115.621 | 2.656 | 23.006 | 1.00 | 29.58 | D |
| ATOM | 6428 | ND2 | ASN D | 358 | 116.439 | 0.916 | 24.171 | 1.00 | 29.80 | D |
| ATOM | 6429 | C | ASN D | 358 | 112.996 | −1.248 | 23.793 | 1.00 | 27.49 | D |
| ATOM | 6430 | O | ASN D | 358 | 113.404 | −1.974 | 24.700 | 1.00 | 27.75 | D |
| ATOM | 6431 | N | ILE D | 359 | 111.732 | −1.217 | 23.408 | 1.00 | 27.57 | D |
| ATOM | 6432 | CA | ILE D | 359 | 110.757 | −2.076 | 24.046 | 1.00 | 28.89 | D |
| ATOM | 6433 | CB | ILE D | 359 | 109.361 | −1.821 | 23.510 | 1.00 | 29.72 | D |
| ATOM | 6434 | CG2 | ILE D | 359 | 108.342 | −2.575 | 24.322 | 1.00 | 28.76 | D |
| ATOM | 6435 | OG1 | ILE D | 359 | 109.058 | −0.336 | 23.581 | 1.00 | 32.82 | D |
| ATOM | 6436 | CD1 | ILE D | 359 | 108.196 | 0.120 | 22.444 | 1.00 | 36.53 | D |
| ATOM | 6437 | C | ILE D | 359 | 111.161 | −3.466 | 23.629 | 1.00 | 28.09 | D |
| ATOM | 6438 | O | ILE D | 359 | 111.164 | −4.389 | 24.425 | 1.00 | 27.60 | D |
| ATOM | 6439 | N | PHE D | 360 | 111.518 | −3.592 | 22.361 | 1.00 | 29.39 | D |
| ATOM | 6440 | CA | PHE D | 360 | 111.926 | −4.864 | 21.810 | 1.00 | 30.15 | D |
| ATOM | 6441 | CB | PHE D | 360 | 112.415 | −4.654 | 20.372 | 1.00 | 32.47 | D |
| ATOM | 6442 | CG | PHE D | 360 | 112.998 | −5.885 | 19.738 | 1.00 | 33.66 | D |
| ATOM | 6443 | CD1 | PHE D | 360 | 112.236 | −7.045 | 19.600 | 1.00 | 34.21 | D |
| ATOM | 6444 | CD2 | PHE D | 360 | 114.327 | −5.898 | 19.326 | 1.00 | 34.66 | D |
| ATOM | 6445 | CE1 | PHE D | 360 | 112.790 | −8.201 | 19.071 | 1.00 | 34.86 | D |
| ATOM | 6446 | CE2 | PHE D | 360 | 114.894 | −7.044 | 18.796 | 1.00 | 36.23 | D |
| ATOM | 6447 | CZ | PHE D | 360 | 114.124 | −8.202 | 18.670 | 1.00 | 35.77 | D |
| ATOM | 6448 | C | PHE D | 360 | 113.015 | −5.503 | 22.654 | 1.00 | 29.32 | D |
| ATOM | 6449 | O | PHE D | 360 | 112.879 | −6.641 | 23.092 | 1.00 | 29.82 | D |
| ATOM | 6450 | N | ILE D | 361 | 114.093 | −4.762 | 22.880 | 1.00 | 28.24 | D |
| ATOM | 6451 | CA | ILE D | 361 | 115.222 | −5.254 | 23.660 | 1.00 | 26.57 | D |
| ATOM | 6452 | CB | ILE D | 361 | 116.376 | −4.219 | 23.597 | 1.00 | 26.56 | D |
| ATOM | 6453 | CG2 | ILE D | 361 | 117.540 | −4.638 | 24.502 | 1.00 | 24.26 | D |
| ATOM | 6454 | CG1 | ILE D | 361 | 116.855 | −4.117 | 22.140 | 1.00 | 25.22 | D |
| ATOM | 6455 | CD1 | ILE D | 361 | 117.841 | −3.000 | 21.868 | 1.00 | 25.88 | D |
| ATOM | 6456 | C | ILE D | 361 | 114.834 | −5.578 | 25.111 | 1.00 | 25.14 | D |
| ATOM | 6457 | O | ILE D | 361 | 115.238 | −6.610 | 25.656 | 1.00 | 24.17 | D |
| ATOM | 6458 | N | LEU D | 362 | 114.027 | −4.712 | 25.718 | 1.00 | 22.80 | D |
| ATOM | 6459 | CA | LEU D | 362 | 113.591 | −4.915 | 27.098 | 1.00 | 23.19 | D |
| ATOM | 6460 | CB | LEU D | 362 | 112.801 | −3.700 | 27.576 | 1.00 | 20.92 | D |
| ATOM | 6461 | CG | LEU D | 362 | 112.565 | −3.604 | 29.077 | 1.00 | 20.66 | D |
| ATOM | 6462 | CD1 | LEU D | 362 | 113.860 | −3.753 | 29.867 | 1.00 | 18.78 | D |
| ATOM | 6463 | CD2 | LEU D | 362 | 111.917 | −2.273 | 29.338 | 1.00 | 20.71 | D |
| ATOM | 6464 | C | LEU D | 362 | 112.773 | −6.196 | 27.330 | 1.00 | 23.63 | D |
| ATOM | 6465 | O | LEU D | 362 | 112.997 | −6.915 | 28.311 | 1.00 | 23.67 | D |
| ATOM | 6466 | N | LYS D | 363 | 111.824 | −6.453 | 26.434 | 1.00 | 23.53 | D |
| ATOM | 6467 | CA | LYS D | 363 | 110.986 | −7.639 | 26.477 | 1.00 | 24.42 | D |
| ATOM | 6468 | CB | LYS D | 363 | 110.156 | −7.743 | 25.191 | 1.00 | 26.43 | D |
| ATOM | 6469 | CG | LYS D | 363 | 108.730 | −7.209 | 25.270 | 1.00 | 30.86 | D |
| ATOM | 6470 | CD | LYS D | 363 | 108.669 | −5.840 | 25.929 | 1.00 | 33.41 | D |
| ATOM | 6471 | CE | LYS D | 363 | 107.226 | −5.421 | 26.252 | 1.00 | 35.72 | D |
| ATOM | 6472 | NZ | LYS D | 363 | 107.204 | −4.294 | 27.264 | 1.00 | 35.50 | D |
| ATOM | 6473 | C | LYS D | 363 | 111.901 | −8.859 | 26.573 | 1.00 | 25.36 | D |
| ATOM | 6474 | O | LYS D | 363 | 111.629 | −9.802 | 27.311 | 1.00 | 25.60 | D |
| ATOM | 6475 | N | ASN D | 364 | 112.988 | −8.829 | 25.809 | 1.00 | 25.23 | D |
| ATOM | 6476 | CA | ASN D | 364 | 113.935 | −9.927 | 25.799 | 1.00 | 25.39 | D |
| ATOM | 6477 | CB | ASN D | 364 | 114.980 | −9.737 | 24.712 | 1.00 | 25.02 | D |
| ATOM | 6478 | CG | ASN D | 364 | 114.436 | −9.985 | 23.348 | 1.00 | 26.12 | D |
| ATOM | 6479 | OD1 | ASN D | 364 | 115.038 | −9.589 | 22.348 | 1.00 | 25.32 | D |
| ATOM | 6480 | ND2 | ASN D | 364 | 113.286 | −10.66 | 223.283 | 1.00 | 27.95 | D |
| ATOM | 6481 | C | ASN D | 364 | 114.640 | −10.030 | 27.118 | 1.00 | 26.63 | D |
| ATOM | 6482 | O | ASN D | 364 | 114.785 | −11.120 | 27.665 | 1.00 | 28.76 | D |
| ATOM | 6483 | N | TRP D | 365 | 115.101 | −8.901 | 27.635 | 1.00 | 26.04 | D |
| ATOM | 6484 | CA | TRP D | 365 | 115.794 | −8.937 | 28.906 | 1.00 | 25.62 | D |
| ATOM | 6485 | CB | TRP D | 365 | 116.211 | −7.535 | 29.357 | 1.00 | 23.36 | D |
| ATOM | 6486 | CG | TRP D | 365 | 117.514 | −7.035 | 28.789 | 1.00 | 19.48 | D |
| ATOM | 6487 | CD2 | TRP D | 365 | 118.801 | −7.069 | 29.427 | 1.00 | 16.99 | D |
| ATOM | 6488 | CE2 | TRP D | 365 | 119.706 | −6.410 | 28.572 | 1.00 | 15.60 | D |
| ATOM | 6489 | CE3 | TRP D | 365 | 119.275 | −7.589 | 30.640 | 1.00 | 15.17 | D |
| ATOM | 6490 | CD1 | TRP D | 365 | 117.691 | −6.387 | 27.613 | 1.00 | 17.39 | D |
| ATOM | 6491 | NE1 | TRP D | 365 | 119.001 | −6.003 | 27.473 | 1.00 | 14.98 | D |
| ATOM | 6492 | CZ2 | TRP D | 365 | 121.072 | −6.249 | 28.892 | 1.00 | 15.36 | D |
| ATOM | 6493 | CZ3 | TRP D | 365 | 120.628 | −7.428 | 30.956 | 1.00 | 13.84 | D |
| ATOM | 6494 | CH2 | TRP D | 365 | 121.509 | −6.762 | 30.081 | 1.00 | 10.82 | D |
| ATOM | 6495 | C | TRP D | 365 | 114.868 | −9.538 | 29.947 | 1.00 | 26.26 | D |
| ATOM | 6496 | O | TRP D | 365 | 115.267 | −10.422 | 30.693 | 1.00 | 28.48 | D |
| ATOM | 6497 | N | TYR D | 366 | 113.621 | −9.084 | 29.956 | 1.00 | 24.80 | D |
| ATOM | 6498 | CA | TYR D | 366 | 112.641 | −9.530 | 30.934 | 1.00 | 23.32 | D |
| ATOM | 6499 | CB | TYR D | 366 | 111.480 | −8.553 | 30.947 | 1.00 | 17.63 | D |
| ATOM | 6500 | CG | TYR D | 366 | 111.793 | −7.211 | 31.597 | 1.00 | 16.02 | D |
| ATOM | 6501 | CD1 | TYR D | 366 | 113.058 | −6.914 | 32.118 | 1.00 | 14.32 | D |
| ATOM | 6502 | CE1 | TYR D | 366 | 113.304 | −5.681 | 32.726 | 1.00 | 11.48 | D |
| ATOM | 6503 | CD2 | TYR D | 366 | 110.808 | −6.243 | 31.712 | 1.00 | 13.79 | D |
| ATOM | 6504 | CE2 | TYR D | 366 | 111.054 | −5.033 | 32.320 | 1.00 | 11.61 | D |
| ATOM | 6505 | CZ | TYR D | 366 | 112.283 | −4.760 | 32.812 | 1.00 | 11.97 | D |
| ATOM | 6506 | OH | TYR D | 366 | 112.475 | −3.532 | 33.364 | 1.00 | 15.79 | D |
| ATOM | 6507 | C | TYR D | 366 | 112.112 | −10.939 | 30.762 | 1.00 | 25.30 | D |
| ATOM | 6508 | O | TYR D | 366 | 111.620 | −11.548 | 31.715 | 1.00 | 25.60 | D |
| ATOM | 6509 | N | ASN D | 367 | 112.227 | −11.441 | 29.538 | 1.00 | 27.79 | D |
| ATOM | 6510 | CA | ASN D | 367 | 111.757 | −12.770 | 29.160 | 1.00 | 28.52 | D |
| ATOM | 6511 | CE | ASN D | 367 | 111.596 | −12.837 | 27.642 | 1.00 | 29.23 | D |
| ATOM | 6512 | CG | ASN D | 367 | 111.047 | −14.162 | 27.168 | 1.00 | 29.51 | D |
| ATOM | 6513 | OD1 | ASN D | 367 | 111.045 | −15.165 | 27.907 | 1.00 | 29.16 | D |
| ATOM | 6514 | ND2 | ASN D | 367 | 110.588 | −14.183 | 25.915 | 1.00 | 28.51 | D |
| ATOM | 6515 | C | ASN D | 367 | 112.706 | −13.872 | 29.617 | 1.00 | 28.04 | D |
| ATOM | 6516 | O | ASN D | 367 | 113.779 | −14.076 | 29.042 | 1.00 | 28.99 | D |
| ATOM | 6517 | N | PRO D | 368 | 112.306 | −14.606 | 30.659 | 1.00 | 27.46 | D |
| ATOM | 6518 | CD | PRO D | 368 | 111.038 | −14.427 | 31.394 | 1.00 | 27.49 | D |
| ATOM | 6519 | CA | PRO D | 368 | 113.094 | −15.701 | 31.228 | 1.00 | 26.35 | D |
| ATOM | 6520 | CB | PRO D | 368 | 112.174 | −16.249 | 32.313 | 1.00 | 26.40 | D |
| ATOM | 6521 | CG | PRO D | 368 | 111.350 | −15.041 | 32.703 | 1.00 | 26.24 | D |
| ATOM | 6522 | C | PRO D | 368 | 113.470 | −16.766 | 30.207 | 1.00 | 25.90 | D |
| ATOM | 6523 | O | PRO D | 368 | 114.556 | −17.338 | 30.270 | 1.00 | 23.69 | D |
| ATOM | 6524 | N | LYS D | 369 | 112.570 | −17.027 | 29.264 | 1.00 | 26.98 | D |
| ATOM | 6525 | CA | LYS D | 369 | 112.815 | −18.051 | 28.249 | 1.00 | 27.69 | D |
| ATOM | 6526 | CB | LYS D | 369 | 111.483 | −18.478 | 27.605 | 1.00 | 27.37 | D |
| ATOM | 6527 | CG | LYS D | 369 | 111.523 | −19.857 | 26.933 | 1.00 | 28.34 | D |
| ATOM | 6528 | CD | LYS D | 369 | 110.159 | −20.280 | 26.431 | 0.00 | 28.71 | D |
| ATOM | 6529 | CE | LYS D | 369 | 109.692 | −19.395 | 25.288 | 0.00 | 29.07 | D |
| ATOM | 6530 | NZ | LYS D | 369 | 108.385 | −19.851 | 24.737 | 0.00 | 29.21 | D |
| ATOM | 6531 | C | LYS D | 369 | 113.815 | −17.602 | 27.170 | 1.00 | 28.21 | D |
| ATOM | 6532 | O | LYS D | 369 | 114.404 | −18.432 | 26.466 | 1.00 | 27.80 | D |
| ATOM | 6533 | N | PHE D | 370 | 114.027 | −16.295 | 27.055 | 1.00 | 28.20 | D |
| ATOM | 6534 | CA | PHE D | 370 | 114.945 | −15.779 | 26.048 | 1.00 | 29.53 | D |
| ATOM | 6535 | CB | PHE D | 370 | 114.804 | −14.267 | 25.930 | 1.00 | 30.24 | D |
| ATOM | 6536 | CG | PHE D | 370 | 115.741 | −13.647 | 24.926 | 1.00 | 31.46 | D |
| ATOM | 6537 | CD1 | PHE D | 370 | 117.107 | −13.497 | 25.208 | 1.00 | 32.17 | D |
| ATOM | 6538 | CD2 | PHE D | 370 | 115.253 | −13.184 | 23.711 | 1.00 | 31.98 | D |
| ATOM | 6539 | CE1 | PHE D | 370 | 117.970 | −12.894 | 24.301 | 1.00 | 30.34 | D |
| ATOM | 6540 | CE2 | PHE D | 370 | 116.105 | −12.580 | 22.795 | 1.00 | 32.40 | D |
| ATOM | 6541 | CZ | PHE D | 370 | 117.473 | −12.434 | 23.098 | 1.00 | 31.69 | D |
| ATOM | 6542 | C | PHE D | 370 | 116.418 | −16.125 | 26.250 | 1.00 | 29.47 | D |
| ATOM | 6543 | O | PHE D | 370 | 116.990 | −15.909 | 27.323 | 1.00 | 30.76 | D |
| ATOM | 6544 | N | VAL D | 371 | 117.038 | −16.623 | 25.186 | 1.00 | 28.02 | D |
| ATOM | 6545 | CA | VAL D | 371 | 118.441 | −16.986 | 25.252 | 1.00 | 27.30 | D |
| ATOM | 6546 | CB | VAL D | 371 | 118.648 | −18.446 | 24.756 | 1.00 | 26.04 | D |
| ATOM | 6547 | CG1 | VAL D | 371 | 120.104 | −18.829 | 24.825 | 1.00 | 22.81 | D |
| ATOM | 6548 | CG2 | VAL D | 371 | 117.827 | −19.396 | 25.624 | 1.00 | 22.71 | D |
| ATOM | 6549 | C | VAL D | 371 | 119.309 | −15.995 | 24.469 | 1.00 | 28.27 | D |
| ATOM | 6550 | O | VAL D | 371 | 119.221 | −15.880 | 23.248 | 1.00 | 27.93 | D |
| ATOM | 6551 | N | PRO D | 372 | 120.161 | −15.251 | 25.188 | 1.00 | 29.70 | D |
| ATOM | 6552 | CD | PRO D | 372 | 120.326 | −15.276 | 26.648 | 1.00 | 29.18 | D |
| ATOM | 6553 | CA | PRO D | 372 | 121.057 | −14.258 | 24.597 | 1.00 | 31.17 | D |
| ATOM | 6554 | CB | PRO D | 372 | 121.744 | −13.642 | 25.816 | 1.00 | 29.97 | D |
| ATOM | 6555 | CG | PRO D | 372 | 120.796 | −13.886 | 26.914 | 1.00 | 30.31 | D |
| ATOM | 6556 | C | PRO D | 372 | 122.071 | −14.884 | 23.659 | 1.00 | 32.23 | D |
| ATOM | 6557 | O | PRO D | 372 | 122.136 | −16.104 | 23.515 | 1.00 | 33.92 | D |
| ATOM | 6558 | N | GLN D | 373 | 122.860 | −14.033 | 23.019 | 1.00 | 32.64 | D |
| ATOM | 6559 | CA | GLN D | 373 | 123.907 | −14.511 | 22.144 | 1.00 | 31.91 | D |
| ATOM | 6560 | CB | GLN D | 373 | 123.945 | −13.701 | 20.865 | 1.00 | 31.03 | D |
| ATOM | 6561 | CG | GLN D | 373 | 124.881 | −14.279 | 19.845 | 1.00 | 31.69 | D |
| ATOM | 6562 | CD | GLN D | 373 | 125.575 | −13.202 | 19.069 | 1.00 | 32.49 | D |
| ATOM | 6563 | OE1 | GLN D | 373 | 124.937 | −12.328 | 18.502 | 1.00 | 33.06 | D |
| ATOM | 6564 | NE2 | GLN D | 373 | 126.892 | −13.251 | 19.045 | 1.00 | 34.46 | D |
| ATOM | 6565 | C | GLN D | 373 | 125.203 | −14.315 | 22.925 | 1.00 | 31.35 | D |
| ATOM | 6566 | O | GLN D | 373 | 125.612 | −13.192 | 23.224 | 1.00 | 31.52 | D |
| ATOM | 6567 | N | ARG D | 374 | 125.842 | −15.418 | 23.264 | 1.00 | 29.71 | D |
| ATOM | 6568 | CA | ARG D | 374 | 127.059 | −15.342 | 24.024 | 1.00 | 29.87 | D |
| ATOM | 6569 | CB | ARG D | 374 | 127.054 | −16.417 | 25.102 | 1.00 | 30.39 | D |
| ATOM | 6570 | CG | ARG D | 374 | 128.347 | −16.511 | 25.854 | 1.00 | 31.03 | D |
| ATOM | 6571 | CD | ARG D | 374 | 128.261 | −17.597 | 26.873 | 1.00 | 31.27 | D |
| ATOM | 6572 | NE | ARG D | 374 | 129.475 | −17.599 | 27.658 | 1.00 | 32.88 | D |
| ATOM | 6573 | CZ | ARG D | 374 | 129.738 | −18.450 | 28.637 | 1.00 | 33.11 | D |
| ATOM | 6574 | NH1 | ARG D | 374 | 128.856 | −19.390 | 28.956 | 1.00 | 31.68 | D |
| ATOM | 6575 | NH2 | ARG D | 374 | 130.889 | −18.343 | 29.301 | 1.00 | 33.83 | D |
| ATOM | 6576 | C | ARG D | 374 | 128.306 | −15.477 | 23.161 | 1.00 | 30.20 | D |
| ATOM | 6577 | O | ARG D | 374 | 128.383 | −16.319 | 22.268 | 1.00 | 30.52 | D |
| ATOM | 6578 | N | THR D | 375 | 129.298 | −14.654 | 23.463 | 1.00 | 30.06 | D |
| ATOM | 6579 | CA | THR D | 375 | 130.543 | −14.641 | 22.724 | 1.00 | 29.07 | D |
| ATOM | 6580 | CB | THR D | 375 | 130.574 | −13.423 | 21.801 | 1.00 | 29.18 | D |
| ATOM | 6581 | OG1 | THR D | 375 | 129.254 | −13.177 | 21.294 | 1.00 | 30.22 | D |
| ATOM | 6582 | CG2 | THR D | 375 | 131.520 | −13.650 | 20.650 | 1.00 | 27.55 | D |
| ATOM | 6583 | C | THR D | 375 | 131.666 | −14.506 | 23.749 | 1.00 | 29.99 | D |
| ATOM | 6584 | O | THR D | 375 | 131.743 | −13.505 | 24.473 | 1.00 | 30.81 | D |
| ATOM | 6585 | N | THR D | 376 | 132.537 | −15.504 | 23.824 | 1.00 | 29.02 | D |
| ATOM | 6586 | CA | THR D | 376 | 133.631 | −15.459 | 24.789 | 1.00 | 28.35 | D |
| ATOM | 6587 | CB | THR D | 376 | 133.775 | −16.810 | 25.551 | 1.00 | 26.89 | D |
| ATOM | 6588 | OG1 | THR D | 376 | 132.516 | −17.174 | 26.129 | 1.00 | 24.67 | D |
| ATOM | 6589 | CG2 | THR D | 376 | 134.822 | −16.700 | 26.648 | 1.00 | 25.06 | D |
| ATOM | 6590 | C | THR D | 376 | 134.959 | −15.155 | 24.120 | 1.00 | 29.32 | D |
| ATOM | 6591 | O | THR D | 376 | 135.386 | −15.886 | 23.234 | 1.00 | 28.86 | D |
| ATOM | 6592 | N | LEU D | 377 | 135.616 | −14.081 | 24.555 | 1.00 | 31.19 | D |
| ATOM | 6593 | CA | LEU D | 377 | 136.916 | −13.701 | 24.001 | 1.00 | 33.04 | D |
| ATOM | 6594 | CS | LEU D | 377 | 136.888 | −12.242 | 23.558 | 1.00 | 31.62 | D |
| ATOM | 6595 | CG | LEU D | 377 | 135.621 | −11.843 | 22.793 | 1.00 | 32.74 | D |
| ATOM | 6596 | CD1 | LEU D | 377 | 135.718 | −10.396 | 22.376 | 1.00 | 32.01 | D |
| ATOM | 6597 | CD2 | LEU D | 377 | 135.430 | −12.735 | 21.590 | 1.00 | 32.02 | D |
| ATOM | 6598 | C | LEU D | 377 | 138.006 | −13.907 | 25.044 | 1.00 | 35.05 | D |
| ATOM | 6599 | O | LEU D | 377 | 137.733 | −13.877 | 26.241 | 1.00 | 36.55 | D |
| ATOM | 6600 | N | ARG D | 378 | 139.238 | −14.122 | 24.590 | 1.00 | 37.30 | D |
| ATOM | 6601 | CA | ARG D | 378 | 140.381 | −14.341 | 25.487 | 1.00 | 38.75 | D |
| ATOM | 6602 | CS | ARG D | 378 | 141.620 | −14.732 | 24.661 | 1.00 | 41.44 | D |
| ATOM | 6603 | CG | ARG D | 378 | 142.735 | −15.467 | 25.412 | 1.00 | 45.29 | D |
| ATOM | 6604 | CD | ARG D | 378 | 143.962 | −15.705 | 24.499 | 1.00 | 49.27 | D |
| ATOM | 6605 | NE | ARG D | 378 | 144.615 | −14.445 | 24.116 | 1.00 | 53.86 | D |
| ATOM | 6606 | CZ | ARG D | 378 | 145.636 | −14.322 | 23.260 | 1.00 | 55.92 | D |
| ATOM | 6607 | NH1 | ARG D | 378 | 146.147 | −15.400 | 22.666 | 1.00 | 55.78 | D |
| ATOM | 6608 | NH2 | ARG D | 378 | 146.152 | −13.109 | 23.009 | 1.00 | 55.08 | D |
| ATOM | 6609 | C | ARG D | 378 | 140.663 | −13.056 | 26.259 | 1.00 | 38.43 | D |
| ATOM | 6610 | O | ARG D | 378 | 140.498 | −11.956 | 25.723 | 1.00 | 37.88 | D |
| ATOM | 6611 | N | GLY D | 379 | 141.098 | −13.206 | 27.510 | 1.00 | 38.54 | D |
| ATOM | 6612 | CA | GLY D | 379 | 141.408 | −12.058 | 28.349 | 1.00 | 38.37 | D |
| ATOM | 6613 | C | GLY D | 379 | 142.901 | −11.817 | 28.563 | 1.00 | 38.46 | D |
| ATOM | 6614 | O | GLY D | 379 | 143.704 | −12.014 | 27.646 | 1.00 | 39.93 | D |
| ATOM | 6615 | N | HIS | D380 | 143.270 | −11.390 | 29.771 | 1.00 | 36.54 | D |
| ATOM | 6616 | CA | HIS D | 380 | 144.663 | −11.116 | 30.118 | 1.00 | 35.40 | D |
| ATOM | 6617 | CB | HIS D | 380 | 144.735 | −10.098 | 31.259 | 1.00 | 32.54 | D |
| ATOM | 6618 | CG | HIS D | 380 | 144.169 | −8.762 | 30.910 | 1.00 | 29.64 | D |
| ATOM | 6619 | CD2 | HIS D | 380 | 144.748 | −7.541 | 30.837 | 1.00 | 29.11 | D |
| ATOM | 6620 | ND1 | HIS D | 380 | 142.845 | −8.580 | 30.581 | 1.00 | 28.66 | D |
| ATOM | 6621 | CE1 | HIS D | 380 | 142.630 | −7.302 | 30.323 | 1.00 | 26.84 | D |
| ATOM | 6622 | NE2 | HIS D | 380 | 143.768 | −6.650 | 30.470 | 1.00 | 28.50 | D |
| ATOM | 6623 | C | HIS D | 380 | 145.400 | −12.389 | 30.524 | 1.00 | 35.86 | D |
| ATOM | 6624 | O | HIS D | 380 | 144.796 | −13.450 | 30.606 | 1.00 | 35.08 | D |
| ATOM | 6625 | N | MSE D | 381 | 146.699 | −12.287 | 30.790 | 1.00 | 37.57 | D |
| ATOM | 6626 | CA | MSE D | 381 | 147.467 | −13.465 | 31.172 | 1.00 | 40.68 | D |
| ATOM | 6627 | CS | MSE D | 381 | 148.937 | −13.253 | 30.850 | 1.00 | 42.82 | D |
| ATOM | 6628 | CG | MSE D | 381 | 149.177 | −13.111 | 29.372 | 1.00 | 45.44 | D |
| ATOM | 6629 | SE | MSE D | 381 | 150.909 | −12.445 | 28.929 | 1.00 | 55.08 | D |
| ATOM | 6630 | CE | MSE D | 381 | 150.582 | −10.533 | 29.001 | 1.00 | 48.67 | D |
| ATOM | 6631 | C | MSE D | 381 | 147.306 | −13.812 | 32.645 | 1.00 | 41.59 | D |
| ATOM | 6632 | O | MSE D | 381 | 147.979 | −14.701 | 33.169 | 1.00 | 40.94 | D |
| ATOM | 6633 | N | THR D | 382 | 146.392 | −13.103 | 33.296 | 1.00 | 42.55 | D |
| ATOM | 6634 | CA | THR D | 382 | 146.091 | −13.286 | 34.704 | 1.00 | 42.91 | D |
| ATOM | 6635 | CB | THR D | 382 | 146.571 | −12.035 | 35.487 | 1.00 | 42.87 | D |
| ATOM | 6636 | OG1 | THR D | 382 | 146.096 | −12.083 | 36.838 | 1.00 | 47.41 | D |
| ATOM | 6637 | CG2 | THR D | 382 | 146.091 | −10.770 | 34.806 | 1.00 | 42.47 | D |
| ATOM | 6638 | C | THR D | 382 | 144.566 | −13.490 | 34.809 | 1.00 | 42.80 | D |
| ATOM | 6639 | O | THR D | 382 | 143.799 | −12.695 | 34.280 | 1.00 | 45.02 | D |
| ATOM | 6640 | N | SER D | 383 | 144.129 | −14.559 | 35.466 | 1.00 | 41.05 | D |
| ATOM | 6641 | CA | SER D | 383 | 142.708 | −14.855 | 35.590 | 1.00 | 40.03 | D |
| ATOM | 6642 | CB | SER D | 383 | 142.527 | −16.201 | 36.285 | 1.00 | 41.63 | D |
| ATOM | 6643 | OG | SER D | 383 | 143.273 | −16.252 | 37.491 | 1.00 | 42.67 | D |
| ATOM | 6644 | C | SER D | 383 | 141.888 | −13.806 | 36.323 | 1.00 | 38.85 | D |
| ATOM | 6645 | O | SER D | 383 | 140.701 | −14.007 | 36.569 | 1.00 | 39.35 | D |
| ATOM | 6646 | N | VAL D | 384 | 142.514 | −12.689 | 36.665 | 1.00 | 37.95 | D |
| ATOM | 6647 | CA | VAL D | 384 | 141.829 | −11.620 | 37.382 | 1.00 | 37.40 | D |
| ATOM | 6648 | CB | VAL D | 384 | 142.471 | −11.414 | 38.777 | 1.00 | 39.34 | D |
| ATOM | 6649 | CG1 | VAL D | 384 | 141.606 | −10.515 | 39.638 | 1.00 | 39.21 | D |
| ATOM | 6650 | CG2 | VAL D | 384 | 142.672 | −12.756 | 39.451 | 1.00 | 41.87 | D |
| ATOM | 6651 | C | VAL D | 384 | 141.899 | −10.302 | 36.615 | 1.00 | 35.77 | D |
| ATOM | 6652 | O | VAL D | 384 | 142.987 | −9.824 | 36.282 | 1.00 | 35.80 | D |
| ATOM | 6653 | N | ILE D | 385 | 140.741 | −9.719 | 36.330 | 1.00 | 33.56 | D |
| ATOM | 6654 | CA | ILE D | 385 | 140.697 | −8.445 | 35.627 | 1.00 | 31.75 | D |
| ATOM | 6655 | CB | ILE D | 385 | 139.503 | −8.347 | 34.670 | 1.00 | 32.21 | D |
| ATOM | 6656 | CG2 | ILE D | 385 | 139.566 | −7.028 | 33.924 | 1.00 | 31.42 | D |
| ATOM | 6657 | CG1 | ILE D | 385 | 139.528 | −9.506 | 33.673 | 1.00 | 34.12 | D |
| ATOM | 6658 | CD1 | ILE D | 385 | 140.728 | −9.519 | 32.787 | 1.00 | 31.79 | D |
| ATOM | 6659 | C | ILE D | 385 | 140.524 | −7.369 | 36.681 | 1.00 | 29.87 | D |
| ATOM | 6660 | O | ILE D | 385 | 139.636 | −7.468 | 37.523 | 1.00 | 29.33 | D |
| ATOM | 6661 | N | THR D | 386 | 141.364 | −6.341 | 36.631 | 1.00 | 28.00 | D |
| ATOM | 6662 | CA | THR D | 386 | 141.284 | −5.262 | 37.603 | 1.00 | 26.59 | D |
| ATOM | 6663 | CB | THR D | 386 | 142.665 | −4.709 | 37.933 | 1.00 | 28.33 | D |
| ATOM | 6664 | OG1 | THR D | 386 | 143.147 | −3.965 | 36.810 | 1.00 | 33.14 | D |
| ATOM | 6665 | CG2 | THR D | 386 | 143.629 | −5.847 | 38.258 | 1.00 | 27.95 | D |
| ATOM | 6666 | C | THR D | 386 | 140.370 | −4.095 | 37.219 | 1.00 | 23.96 | D |
| ATOM | 6667 | O | THR D | 386 | 140.049 | −3.258 | 38.057 | 1.00 | 23.82 | D |
| ATOM | 6668 | N | CYS D | 387 | 139.969 | −4.000 | 35.959 | 1.00 | 21.99 | D |
| ATOM | 6669 | CA | CYS D | 387 | 139.032 | −2.946 | 35.583 | 1.00 | 21.32 | D |
| ATOM | 6670 | CB | CYS D | 387 | 139.593 | −1.554 | 35.810 | 1.00 | 21.52 | D |
| ATOM | 6671 | SG | CYS D | 387 | 141.286 | −1.370 | 35.494 | 1.00 | 24.48 | D |
| ATOM | 6672 | C | CYS D | 387 | 138.522 | −3.058 | 34.185 | 1.00 | 20.32 | D |
| ATOM | 6673 | O | CYS D | 387 | 139.180 | −3.609 | 33.309 | 1.00 | 20.62 | D |
| ATOM | 6674 | N | LEU D | 388 | 137.313 | −2.554 | 33.993 | 1.00 | 21.13 | D |
| ATOM | 6675 | CA | LEU D | 388 | 136.666 | −2.620 | 32.699 | 1.00 | 21.99 | D |
| ATOM | 6676 | CB | LEU D | 388 | 135.720 | −3.811 | 32.699 | 1.00 | 19.44 | D |
| ATOM | 6677 | CG | LEU D | 388 | 134.666 | −3.896 | 31.603 | 1.00 | 17.83 | D |
| ATOM | 6678 | CD1 | LEU D | 388 | 134.369 | −5.364 | 31.382 | 1.00 | 19.58 | D |
| ATOM | 6679 | CD2 | LEU D | 388 | 133.406 | −3.172 | 32.009 | 1.00 | 17.27 | D |
| ATOM | 6680 | C | LEU D | 388 | 135.910 | −1.349 | 32.307 | 1.00 | 23.54 | D |
| ATOM | 6681 | O | LEU D | 388 | 135.262 | −0.724 | 33.136 | 1.00 | 24.86 | D |
| ATOM | 6682 | N | GLN D | 389 | 136.011 | −0.965 | 31.038 | 1.00 | 25.33 | D |
| ATOM | 6683 | CA | GLN D | 389 | 135.302 | 0.203 | 30.521 | 1.00 | 25.28 | D |
| ATOM | 6684 | CB | GLN D | 389 | 136.275 | 1.296 | 30.073 | 1.00 | 25.97 | D |
| ATOM | 6685 | CG | GLN D | 389 | 137.029 | 2.071 | 31.150 | 1.00 | 25.10 | D |
| ATOM | 6686 | CD | GLN D | 389 | 136.179 | 3.114 | 31.877 | 1.00 | 26.41 | D |
| ATOM | 6687 | OE1 | GLN D | 389 | 135.673 | 2.850 | 32.949 | 1.00 | 26.25 | D |
| ATOM | 6688 | NE2 | GLN D | 389 | 136.031 | 4.304 | 31.293 | 1.00 | 27.94 | D |
| ATOM | 6689 | C | GLN D | 389 | 134.477 | −0.233 | 29.303 | 1.00 | 26.25 | D |
| ATOM | 6690 | O | GLN D | 389 | 134.917 | −1.076 | 28.515 | 1.00 | 26.39 | D |
| ATOM | 6691 | N | PHE D | 390 | 133.284 | 0.340 | 29.153 | 1.00 | 27.94 | D |
| ATOM | 6692 | CA | PHE D | 390 | 132.415 | 0.045 | 28.015 | 1.00 | 28.91 | D |
| ATOM | 6693 | CB | PHE D | 390 | 131.364 | −0.995 | 28.424 | 1.00 | 26.05 | D |
| ATOM | 6694 | CG | PHE D | 390 | 130.416 | −1.381 | 27.322 | 1.00 | 26.58 | D |
| ATOM | 6695 | CD1 | PHE D | 390 | 130.826 | −1.411 | 25.998 | 1.00 | 27.31 | D |
| ATOM | 6696 | CD2 | PHE D | 390 | 129.087 | −1.675 | 27.609 | 1.00 | 27.25 | D |
| ATOM | 6697 | CE1 | PHE D | 390 | 129.921 | −1.716 | 24.978 | 1.00 | 26.45 | D |
| ATOM | 6698 | CE2 | PHE D | 390 | 128.179 | −1.982 | 26.595 | 1.00 | 26.66 | D |
| ATOM | 6699 | CZ | PHE D | 390 | 128.597 | −1.999 | 25.284 | 1.00 | 25.88 | D |
| ATOM | 6700 | C | PHE D | 390 | 131.800 | 1.375 | 27.521 | 1.00 | 31.17 | D |
| ATOM | 6701 | O | PHE D | 390 | 130.844 | 1.911 | 28.095 | 1.00 | 31.73 | D |
| ATOM | 6702 | N | GLU D | 391 | 132.404 | 1.908 | 26.460 | 1.00 | 33.23 | D |
| ATOM | 6703 | CA | GLU D | 391 | 132.004 | 3.172 | 25.842 | 1.00 | 36.52 | D |
| ATOM | 6704 | CB | GLU D | 391 | 132.752 | 4.335 | 26.494 | 1.00 | 37.25 | D |
| ATOM | 6705 | CG | GLU D | 391 | 131.870 | 5.425 | 27.041 | 1.00 | 38.77 | D |
| ATOM | 6706 | CD | GLU D | 391 | 131.258 | 5.032 | 28.354 | 1.00 | 39.09 | D |
| ATOM | 6707 | OE1 | GLU D | 391 | 132.023 | 4.888 | 29.322 | 1.00 | 40.49 | D |
| ATOM | 6708 | OE2 | GLU D | 391 | 130.024 | 4.859 | 28.417 | 1.00 | 39.48 | D |
| ATOM | 6709 | C | GLU D | 391 | 132.377 | 3.150 | 24.360 | 1.00 | 37.92 | D |
| ATOM | 6710 | O | GLU D | 391 | 133.293 | 2.426 | 23.956 | 1.00 | 39.21 | D |
| ATOM | 6711 | N | ASP D | 392 | 131.704 | 3.971 | 23.557 | 1.00 | 38.58 | D |
| ATOM | 6712 | CA | ASP D | 392 | 131.986 | 4.053 | 22.112 | 1.00 | 38.07 | D |
| ATOM | 6713 | CB | ASP D | 392 | 133.157 | 5.009 | 21.858 | 1.00 | 37.43 | D |
| ATOM | 6714 | CG | ASP D | 392 | 132.716 | 6.428 | 21.778 | 1.00 | 39.32 | D |
| ATOM | 6715 | OD1 | ASP D | 392 | 131.501 | 6.650 | 21.954 | 1.00 | 40.76 | D |
| ATOM | 6716 | OD2 | ASP D | 392 | 133.557 | 7.317 | 21.530 | 1.00 | 40.53 | D |
| ATOM | 6717 | C | ASP D | 392 | 132.253 | 2.725 | 21.400 | 1.00 | 36.82 | D |
| ATOM | 6718 | O | ASP D | 392 | 133.221 | 2.589 | 20.647 | 1.00 | 36.96 | D |
| ATOM | 6719 | N | ASN D | 393 | 131.381 | 1.753 | 21.631 | 1.00 | 35.81 | D |
| ATOM | 6720 | CA | ASN D | 393 | 131.513 | 0.448 | 21.000 | 1.00 | 36.51 | D |
| ATOM | 6721 | CB | ASN D | 393 | 131.410 | 0.572 | 19.471 | 1.00 | 37.62 | D |
| ATOM | 6722 | CG | ASN D | 393 | 130.111 | 1.264 | 19.001 | 1.00 | 39.78 | D |
| ATOM | 6723 | OD1 | ASN D | 393 | 130.023 | 1.696 | 17.846 | 1.00 | 41.53 | D |
| ATOM | 6724 | ND2 | ASN D | 393 | 129.111 | 1.362 | 19.881 | 1.00 | 38.28 | D |
| ATOM | 6725 | C | ASN D | 393 | 132.829 | −0.215 | 21.366 | 1.00 | 34.65 | D |
| ATOM | 6726 | O | ASN D | 393 | 133.249 | −1.163 | 20.710 | 1.00 | 34.87 | D |
| ATOM | 6727 | N | TYR D | 394 | 133.477 | 0.292 | 22.409 | 1.00 | 33.42 | D |
| ATOM | 6728 | CA | TYR D | 394 | 134.742 | −0.264 | 22.884 | 1.00 | 32.37 | D |
| ATOM | 6729 | CB | TYR D | 394 | 135.783 | 0.838 | 23.102 | 1.00 | 32.07 | D |
| ATOM | 6730 | CG | TYR D | 394 | 136.458 | 1.349 | 21.860 | 1.00 | 32.95 | D |
| ATOM | 6731 | CD1 | TYR D | 394 | 137.124 | 0.479 | 20.992 | 1.00 | 32.58 | D |
| ATOM | 6732 | CE1 | TYR D | 394 | 137.777 | 0.953 | 19.858 | 1.00 | 31.50 | D |
| ATOM | 6733 | CD2 | TYR D | 394 | 136.459 | 2.704 | 21.565 | 1.00 | 31.89 | D |
| ATOM | 6734 | CE2 | TYR D | 394 | 137.104 | 3.189 | 20.445 | 1.00 | 33.06 | D |
| ATOM | 6735 | CZ | TYR D | 394 | 137.762 | 2.316 | 19.594 | 1.00 | 33.32 | D |
| ATOM | 6736 | OH | TYR D | 394 | 138.406 | 2.827 | 18.492 | 1.00 | 33.69 | D |
| ATOM | 6737 | C | TYR D | 394 | 134.554 | −0.956 | 24.224 | 1.00 | 32.16 | D |
| ATOM | 6738 | O | TYR D | 394 | 133.692 | −0.582 | 25.016 | 1.00 | 33.01 | D |
| ATOM | 6739 | N | VAL D | 395 | 135.359 | −1.970 | 24.481 | 1.00 | 31.18 | D |
| ATOM | 6740 | CA | VAL D | 395 | 135.307 | −2.636 | 25.767 | 1.00 | 30.34 | D |
| ATOM | 6741 | CB | VAL D | 395 | 134.724 | −4.036 | 25.689 | 1.00 | 32.30 | D |
| ATOM | 6742 | CG1 | VAL D | 395 | 134.537 | −4.571 | 27.092 | 1.00 | 30.09 | D |
| ATOM | 6743 | CG2 | VAL D | 395 | 133.388 | −4.017 | 24.945 | 1.00 | 32.97 | D |
| ATOM | 6744 | C | VAL D | 395 | 136.751 | −2.752 | 26.150 | 1.00 | 29.50 | D |
| ATOM | 6745 | O | VAL D | 395 | 137.469 | −3.592 | 25.608 | 1.00 | 30.47 | D |
| ATOM | 6746 | N | ILE D | 396 | 137.190 | −1.895 | 27.061 | 1.00 | 27.33 | D |
| ATOM | 6747 | CA | ILE D | 396 | 138.579 | −1.929 | 27.471 | 1.00 | 24.79 | D |
| ATOM | 6748 | CB | ILE D | 396 | 139.202 | −0.529 | 27.477 | 1.00 | 20.54 | D |
| ATOM | 6749 | CG2 | ILE D | 396 | 140.724 | −0.654 | 27.607 | 1.00 | 18.18 | D |
| ATOM | 6750 | CG1 | ILE D | 396 | 138.779 | 0.231 | 26.222 | 1.00 | 19.10 | D |
| ATOM | 6751 | CD1 | ILE D | 396 | 137.698 | 1.261 | 26.480 | 1.00 | 15.50 | D |
| ATOM | 6752 | C | ILE D | 396 | 138.721 | −2.532 | 28.860 | 1.00 | 25.91 | D |
| ATOM | 6753 | O | ILE D | 396 | 137.839 | −2.371 | 29.709 | 1.00 | 25.28 | D |
| ATOM | 6754 | N | THR D | 397 | 139.839 | −3.228 | 29.075 | 1.00 | 25.38 | D |
| ATOM | 6755 | CA | THR D | 397 | 140.133 | −3.867 | 30.352 | 1.00 | 24.35 | D |
| ATOM | 6756 | CB | THR D | 397 | 139.734 | −5.353 | 30.367 | 1.00 | 23.41 | D |
| ATOM | 6757 | OG1 | THR D | 397 | 140.542 | −6.067 | 29.425 | 1.00 | 22.79 | D |
| ATOM | 6758 | CG2 | THR D | 397 | 138.280 | −5.530 | 30.010 | 1.00 | 22.25 | D |
| ATOM | 6759 | C | THR D | 397 | 141.610 | −3.834 | 30.732 | 1.00 | 25.66 | D |
| ATOM | 6760 | O | THR D | 397 | 142.492 | −3.945 | 29.876 | 1.00 | 25.23 | D |
| ATOM | 6761 | N | GLY D | 398 | 141.871 | −3.715 | 32.032 | 1.00 | 26.89 | D |
| ATOM | 6762 | CA | GLY D | 398 | 143.239 | −3.717 | 32.535 | 1.00 | 27.00 | D |
| ATOM | 6763 | C | GLY D | 398 | 143.440 | −4.892 | 33.487 | 1.00 | 26.90 | D |
| ATOM | 6764 | O | GLY D | 398 | 142.464 | −5.481 | 33.951 | 1.00 | 26.42 | D |
| ATOM | 6765 | N | ALA D | 399 | 144.692 | −5.250 | 33.776 | 1.00 | 28.42 | D |
| ATOM | 6766 | CA | ALA D | 399 | 144.977 | −6.367 | 34.694 | 1.00 | 29.91 | D |
| ATOM | 6767 | CB | ALA D | 399 | 144.889 | −7.701 | 33.945 | 1.00 | 28.40 | D |
| ATOM | 6768 | C | ALA D | 399 | 146.335 | −6.265 | 35.410 | 1.00 | 31.06 | D |
| ATOM | 6769 | O | ALA D | 399 | 147.208 | −5.469 | 35.041 | 1.00 | 29.92 | D |
| ATOM | 6770 | N | ASP D | 400 | 146.511 | −7.083 | 36.444 | 1.00 | 33.65 | D |
| ATOM | 6771 | CA | ASP D | 400 | 147.767 | −7.066 | 37.178 | 1.00 | 35.36 | D |
| ATOM | 6772 | CB | ASP D | 400 | 147.670 | −7.891 | 38.473 | 1.00 | 37.51 | D |
| ATOM | 6773 | CG | ASP D | 400 | 148.845 | −7.626 | 39.429 | 1.00 | 42.78 | D |
| ATOM | 6774 | OD1 | ASP D | 400 | 149.073 | −6.446 | 39.813 | 1.00 | 42.00 | D |
| ATOM | 6775 | OD2 | ASP D | 400 | 149.544 | −8.603 | 39.802 | 1.00 | 45.77 | D |
| ATOM | 6776 | C | ASP D | 400 | 148.818 | −7.639 | 36.237 | 1.00 | 34.42 | D |
| ATOM | 6777 | O | ASP D | 400 | 149.996 | −7.735 | 36.579 | 1.00 | 34.18 | D |
| ATOM | 6778 | N | ASP D | 401 | 148.379 | −8.014 | 35.037 | 1.00 | 33.68 | D |
| ATOM | 6779 | CA | ASP D | 401 | 149.293 | −8.550 | 34.047 | 1.00 | 32.54 | D |
| ATOM | 6780 | CB | ASP D | 401 | 148.571 | −9.554 | 33.118 | 1.00 | 31.92 | D |
| ATOM | 6781 | CG | ASP D | 401 | 147.897 | −8.910 | 31.909 | 1.00 | 31.59 | D |
| ATOM | 6782 | OD1 | ASP D | 401 | 147.579 | −7.704 | 31.945 | 1.00 | 30.42 | D |
| ATOM | 6783 | OD2 | ASP D | 401 | 147.671 | −9.645 | 30.916 | 1.00 | 31.10 | D |
| ATOM | 6784 | C | ASP D | 401 | 149.901 | −7.372 | 33.293 | 1.00 | 32.98 | D |
| ATOM | 6785 | O | ASP D | 401 | 150.551 | −7.539 | 32.266 | 1.00 | 33.47 | D |
| ATOM | 6786 | N | LYS D | 402 | 149.699 | −6.180 | 33.854 | 1.00 | 33.97 | D |
| ATOM | 6787 | CA | LYS D | 402 | 150.215 | −4.921 | 33.319 | 1.00 | 34.64 | D |
| ATOM | 6788 | CB | LYS D | 402 | 151.735 | −4.825 | 33.568 | 1.00 | 33.75 | D |
| ATOM | 6789 | CG | LYS D | 402 | 152.545 | −5.988 | 33.073 | 1.00 | 33.34 | D |
| ATOM | 6790 | CD | LYS D | 402 | 153.625 | −6.377 | 34.058 | 1.00 | 34.65 | D |
| ATOM | 6791 | CE | LYS D | 402 | 154.661 | −5.300 | 34.221 | 1.00 | 35.08 | D |
| ATOM | 6792 | NZ | LYS D | 402 | 155.714 | −5.811 | 35.142 | 1.00 | 34.72 | D |
| ATOM | 6793 | C | LYS D | 402 | 149.895 | −4.650 | 31.846 | 1.00 | 35.07 | D |
| ATOM | 6794 | O | LYS D | 402 | 150.606 | −3.907 | 31.167 | 1.00 | 35.57 | D |
| ATOM | 6795 | N | MSE D | 403 | 148.809 | −5.238 | 31.360 | 1.00 | 37.46 | D |
| ATOM | 6796 | CA | MSE D | 403 | 148.389 | −5.066 | 29.966 | 1.00 | 39.42 | D |
| ATOM | 6797 | CB | MSE D | 403 | 148.429 | −6.405 | 29.210 | 1.00 | 40.61 | D |
| ATOM | 6798 | CG | MSE D | 403 | 149.792 | −7.054 | 29.119 | 1.00 | 42.82 | D |
| ATOM | 6799 | SE | MSE D | 403 | 150.956 | −5.997 | 28.018 | 1.00 | 47.96 | D |
| ATOM | 6800 | CE | MSE D | 403 | 152.415 | −5.649 | 29.261 | 1.00 | 46.83 | D |
| ATOM | 6801 | C | MSE D | 403 | 146.970 | −4.518 | 29.882 | 1.00 | 39.22 | D |
| ATOM | 6802 | O | MSE D | 403 | 146.140 | −4.767 | 30.761 | 1.00 | 41.64 | D |
| ATOM | 6803 | N | ILE D | 404 | 146.691 | −3.797 | 28.802 | 1.00 | 37.92 | D |
| ATOM | 6804 | CA | ILE D | 404 | 145.372 | −3.216 | 28.582 | 1.00 | 35.96 | O |
| ATOM | 6805 | CB | ILE D | 404 | 145.455 | −1.675 | 28.574 | 1.00 | 34.69 | O |
| ATOM | 6806 | CG2 | ILE D | 404 | 144.114 | −1.082 | 28.156 | 1.00 | 33.19 | D |
| ATOM | 6807 | CG1 | ILE D | 404 | 145.880 | −1.186 | 29.962 | 1.00 | 33.10 | D |
| ATOM | 6808 | CO1 | ILE D | 404 | 146.272 | 0.266 | 29.999 | 1.00 | 31.33 | O |
| ATOM | 6809 | C | ILE D | 404 | 144.788 | −3.709 | 27.251 | 1.00 | 35.14 | D |
| ATOM | 6810 | O | ILE D | 404 | 145.304 | −3.392 | 26.193 | 1.00 | 34.48 | O |
| ATOM | 6811 | N | ARG D | 405 | 143.709 | −4.482 | 27.325 | 1.00 | 34.81 | D |
| ATOM | 6812 | CA | ARG D | 405 | 143.046 | −5.027 | 26.151 | 1.00 | 34.51 | O |
| ATOM | 6813 | CB | ARG D | 405 | 142.428 | −6.373 | 26.494 | 1.00 | 34.25 | O |
| ATOM | 6814 | CG | ARG D | 405 | 143.353 | −7.285 | 27.227 | 1.00 | 34.29 | O |
| ATOM | 6815 | CD | ARG D | 405 | 144.147 | −8.101 | 26.302 | 1.00 | 33.92 | O |
| ATOM | 6816 | NE | ARG D | 405 | 143.362 | −9.187 | 25.745 | 1.00 | 34.85 | O |
| ATOM | 6817 | CZ | ARG D | 405 | 143.844 | −10.076 | 24.882 | 1.00 | 37.01 | D |
| ATOM | 6818 | NH1 | ARG D | 405 | 145.110 | −9.992 | 24.481 | 1.00 | 32.55 | D |
| ATOM | 6819 | NH2 | ARG D | 405 | 143.069 | −11.063 | 24.436 | 1.00 | 38.58 | D |
| ATOM | 6820 | C | ARG D | 405 | 141.931 | −4.099 | 25.668 | 1.00 | 35.40 | O |
| ATOM | 6821 | O | ARG D | 405 | 141.238 | −3.474 | 26.479 | 1.00 | 36.54 | D |
| ATOM | 6822 | N | VAL D | 406 | 141.749 | −4.041 | 24.349 | 1.00 | 35.29 | D |
| ATOM | 6823 | CA | VAL D | 406 | 140.719 | −3.214 | 23.719 | 1.00 | 34.73 | D |
| ATOM | 6824 | CB | VAL D | 406 | 141.332 | −2.093 | 22.879 | 1.00 | 35.48 | D |
| ATOM | 6825 | CG1 | VAL D | 406 | 140.229 | −1.244 | 22.275 | 1.00 | 35.19 | D |
| ATOM | 6826 | CG2 | VAL D | 406 | 142.275 | −1.253 | 23.737 | 1.00 | 35.51 | O |
| ATOM | 6827 | C | VAL D | 406 | 139.921 | −4.085 | 22.780 | 1.00 | 34.43 | O |
| ATOM | 6828 | O | VAL D | 406 | 140.500 | −4.664 | 21.874 | 1.00 | 34.33 | D |
| ATOM | 6829 | N | TYR D | 407 | 138.603 | −4.160 | 22.977 | 1.00 | 34.53 | D |
| ATOM | 6830 | CA | TYR D | 407 | 137.739 | −4.985 | 22.124 | 1.00 | 34.18 | D |
| ATOM | 6831 | CB | TYR D | 407 | 137.059 | −6.091 | 22.937 | 1.00 | 33.29 | D |
| ATOM | 6832 | CG | TYR D | 407 | 137.966 | −6.948 | 23.781 | 1.00 | 33.16 | D |
| ATOM | 6833 | CD1 | TYR D | 407 | 138.423 | −6.509 | 25.026 | 1.00 | 32.93 | D |
| ATOM | 6834 | CE1 | TYR D | 407 | 139.255 | −7.313 | 25.802 | 1.00 | 33.43 | D |
| ATOM | 6835 | CD2 | TYR D | 407 | 138.362 | −8.206 | 23.335 | 1.00 | 31.85 | D |
| ATOM | 6836 | CE2 | TYR D | 407 | 139.189 | −9.013 | 24.091 | 1.00 | 31.74 | D |
| ATOM | 6837 | CZ | TYR D | 407 | 139.637 | −8.572 | 25.320 | 1.00 | 33.54 | O |
| ATOM | 6838 | OH | TYR D | 407 | 140.485 | −9.378 | 26.052 | 1.00 | 34.77 | D |
| ATOM | 6839 | C | TYR D | 407 | 136.635 | −4.194 | 21.417 | 1.00 | 34.58 | D |
| ATOM | 6840 | O | TYR D | 407 | 136.246 | −3.117 | 21.864 | 1.00 | 35.32 | D |
| ATOM | 6841 | N | ASP D | 408 | 136.122 | −4.775 | 20.332 | 1.00 | 35.38 | D |
| ATOM | 6842 | CA | ASP D | 408 | 135.043 | −4.221 | 19.501 | 1.00 | 35.74 | D |
| ATOM | 6843 | CB | ASP D | 408 | 135.302 | −4.625 | 18.038 | 1.00 | 38.38 | D |
| ATOM | 6844 | CG | ASP D | 408 | 134.214 | −4.143 | 17.066 | 1.00 | 40.45 | D |
| ATOM | 6845 | OD1 | ASP D | 408 | 133.015 | −4.143 | 17.433 | 1.00 | 41.61 | D |
| ATOM | 6846 | OD2 | ASP D | 408 | 134.563 | −3.781 | 15.917 | 1.00 | 39.05 | D |
| ATOM | 6847 | C | ASP D | 408 | 133.685 | −4.788 | 19.965 | 1.00 | 35.67 | D |
| ATOM | 6848 | O | ASP D | 408 | 133.413 | −5.980 | 19.799 | 1.00 | 35.45 | D |
| ATOM | 6849 | N | SER D | 409 | 132.829 | −3.934 | 20.519 | 1.00 | 36.24 | D |
| ATOM | 6850 | CA | SER D | 409 | 131.510 | −4.351 | 21.021 | 1.00 | 37.97 | D |
| ATOM | 6851 | CB | SER D | 409 | 130.779 | −3.160 | 21.623 | 1.00 | 39.00 | D |
| ATOM | 6852 | OG | SER D | 409 | 131.590 | −2.452 | 22.533 | 1.00 | 42.40 | D |
| ATOM | 6853 | C | SER D | 409 | 130.594 | −4.953 | 19.968 | 1.00 | 39.04 | D |
| ATOM | 6854 | O | SER D | 409 | 129.915 | −5.958 | 20.216 | 1.00 | 38.64 | D |
| ATOM | 6855 | N | ILE D | 410 | 130.565 | −4.295 | 18.807 | 1.00 | 40.27 | D |
| ATOM | 6856 | CA | ILE D | 410 | 129.740 | −4.695 | 17.658 | 1.00 | 41.19 | D |
| ATOM | 6857 | CB | ILE D | 410 | 129.743 | −3.613 | 16.553 | 1.00 | 42.29 | D |
| ATOM | 6858 | CG2 | ILE D | 410 | 128.654 | −3.904 | 15.539 | 1.00 | 41.48 | D |
| ATOM | 6859 | CG1 | ILE D | 410 | 129.507 | −2.227 | 17.162 | 1.00 | 43.94 | D |
| ATOM | 6860 | CD1 | ILE D | 410 | 129.838 | −1.089 | 16.202 | 1.00 | 43.04 | D |
| ATOM | 6861 | C | ILE D | 410 | 130.244 | −5.996 | 17.038 | 1.00 | 40.21 | D |
| ATOM | 6862 | O | ILE D | 410 | 129.616 | −7.041 | 17.170 | 1.00 | 40.47 | D |
| ATOM | 6863 | N | ASN D | 411 | 131.385 | −5.945 | 16.370 | 1.00 | 38.97 | D |
| ATOM | 6864 | CA | ASN D | 411 | 131.900 | −7.153 | 15.769 | 1.00 | 39.16 | D |
| ATOM | 6865 | CB | ASN D | 411 | 133.158 | −6.848 | 14.985 | 1.00 | 41.69 | D |
| ATOM | 6866 | CG | ASN D | 411 | 132.943 | −5.731 | 13.995 | 1.00 | 43.77 | D |
| ATOM | 6867 | OD1 | ASN D | 411 | 131.889 | −5.649 | 13.355 | 1.00 | 43.07 | D |
| ATOM | 6868 | ND2 | ASN D | 411 | 133.938 | −4.866 | 13.855 | 1.00 | 45.58 | D |
| ATOM | 6869 | C | ASN D | 411 | 132.178 | −8.213 | 16.805 | 1.00 | 38.20 | D |
| ATOM | 6870 | O | ASN D | 411 | 132.364 | −9.377 | 16.458 | 1.00 | 36.34 | D |
| ATOM | 6871 | N | LYS D | 412 | 132.197 | −7.800 | 18.074 | 1.00 | 37.74 | D |
| ATOM | 6872 | CA | LYS D | 412 | 132.451 | −8.693 | 19.211 | 1.00 | 36.63 | D |
| ATOM | 6873 | CB | LYS D | 412 | 131.300 | −9.690 | 19.338 | 1.00 | 35.83 | D |
| ATOM | 6874 | CG | LYS D | 412 | 129.936 | −9.018 | 19.314 | 1.00 | 35.16 | D |
| ATOM | 6875 | CD | LYS D | 412 | 128.805 | −10.011 | 19.175 | 1.00 | 34.88 | D |
| ATOM | 6876 | CE | LYS D | 412 | 127.483 | −9.278 | 19.119 | 1.00 | 36.26 | D |
| ATOM | 6877 | NZ | LYS D | 412 | 126.321 | −10.211 | 19.101 | 1.00 | 37.05 | D |
| ATOM | 6878 | C | LYS D | 412 | 133.776 | −9.442 | 19.066 | 1.00 | 36.14 | D |
| ATOM | 6879 | O | LYS D | 412 | 133.828 | −10.659 | 19.227 | 1.00 | 35.34 | D |
| ATOM | 6880 | N | LYS D | 413 | 134.845 | −8.707 | 18.772 | 1.00 | 36.48 | D |
| ATOM | 6881 | CA | LYS D | 413 | 136.161 | −9.314 | 18.597 | 1.00 | 38.12 | D |
| ATOM | 6882 | CB | LYS D | 413 | 136.440 | −9.573 | 17.118 | 1.00 | 37.83 | D |
| ATOM | 6883 | CG | LYS D | 413 | 136.378 | −8.322 | 16.257 | 1.00 | 41.43 | D |
| ATOM | 6884 | CD | LYS D | 413 | 136.964 | −8.566 | 14.853 | 1.00 | 44.72 | D |
| ATOM | 6885 | CE | LYS D | 413 | 136.674 | −7.400 | 13.885 | 1.00 | 46.65 | D |
| ATOM | 6886 | NZ | LYS D | 413 | 137.132 | −6.056 | 14.371 | 1.00 | 47.55 | D |
| ATOM | 6887 | C | LYS D | 413 | 137.303 | −8.471 | 19.159 | 1.00 | 38.78 | D |
| ATOM | 6888 | O | LYS D | 413 | 137.244 | −7.242 | 19.151 | 1.00 | 38.60 | D |
| ATOM | 6889 | N | PHE D | 414 | 138.348 | −9.149 | 19.630 | 1.00 | 38.98 | D |
| ATOM | 6890 | CA | PHE D | 414 | 139.520 | −8.489 | 20.191 | 1.00 | 38.98 | D |
| ATOM | 6891 | CB | PHE D | 414 | 140.513 | −9.528 | 20.715 | 1.00 | 39.56 | D |
| ATOM | 6892 | CG | PHE D | 414 | 141.908 | −8.997 | 20.902 | 1.00 | 41.07 | D |
| ATOM | 6893 | CD1 | PHE D | 414 | 142.184 | −8.046 | 21.874 | 1.00 | 43.51 | D |
| ATOM | 6894 | CD2 | PHE D | 414 | 142.949 | −9.454 | 20.110 | 1.00 | 41.71 | D |
| ATOM | 6895 | CE1 | PHE D | 414 | 143.488 | −7.557 | 22.058 | 1.00 | 44.32 | D |
| ATOM | 6896 | CE2 | PHE D | 414 | 144.251 | −8.973 | 20.287 | 1.00 | 43.60 | D |
| ATOM | 6897 | CZ | PHE D | 414 | 144.519 | −8.022 | 21.264 | 1.00 | 42.78 | D |
| ATOM | 6898 | C | PHE D | 414 | 140.208 | −7.620 | 19.157 | 1.00 | 39.17 | D |
| ATOM | 6899 | O | PHE D | 414 | 140.529 | −8.083 | 18.068 | 1.00 | 40.84 | D |
| ATOM | 6900 | N | LEU D | 415 | 140.430 | −6.359 | 19.506 | 1.00 | 38.34 | D |
| ATOM | 6901 | CA | LEU D | 415 | 141.099 | −5.434 | 18.617 | 1.00 | 37.48 | D |
| ATOM | 6902 | CB | LEU D | 415 | 140.555 | −4.026 | 18.815 | 1.00 | 37.15 | D |
| ATOM | 6903 | CG | LEU D | 415 | 139.163 | −3.792 | 18.224 | 1.00 | 38.52 | D |
| ATOM | 6904 | CD1 | LEU D | 415 | 138.737 | −2.351 | 18.453 | 1.00 | 39.38 | D |
| ATOM | 6905 | CD2 | LEU D | 415 | 139.191 | −4.091 | 16.736 | 1.00 | 39.17 | D |
| ATOM | 6906 | C | LEU D | 415 | 142.611 | −5.451 | 18.832 | 1.00 | 37.67 | D |
| ATOM | 6907 | O | LEU D | 415 | 143.328 | −6.171 | 18.138 | 1.00 | 38.98 | D |
| ATOM | 6908 | N | LEU D | 416 | 143.097 | −4.681 | 19.799 | 1.00 | 35.95 | D |
| ATOM | 6909 | CA | LEU D | 416 | 144.528 | −4.611 | 20.052 | 1.00 | 34.61 | D |
| ATOM | 6910 | CB | LEU D | 416 | 145.102 | −3.394 | 19.317 | 1.00 | 32.51 | D |
| ATOM | 6911 | CG | LEU D | 416 | 144.656 | −2.045 | 19.880 | 1.00 | 30.50 | D |
| ATOM | 6912 | CD1 | LEU D | 416 | 145.700 | −1.579 | 20.853 | 1.00 | 31.53 | D |
| ATOM | 6913 | CD2 | LEU D | 416 | 144.470 | −1.017 | 18.789 | 1.00 | 30.55 | D |
| ATOM | 6914 | C | LEU D | 416 | 144.865 | −4.519 | 21.545 | 1.00 | 35.53 | D |
| ATOM | 6915 | O | LEU D | 416 | 144.013 | −4.200 | 22.364 | 1.00 | 36.55 | D |
| ATOM | 6916 | N | GLN D | 417 | 146.116 | −4.791 | 21.891 | 1.00 | 35.81 | D |
| ATOM | 6917 | CA | GLN D | 417 | 146.547 | −4.716 | 23.271 | 1.00 | 35.57 | D |
| ATOM | 6918 | CB | GLN D | 417 | 147.254 | −6.007 | 23.671 | 1.00 | 37.43 | D |
| ATOM | 6919 | CG | GLN D | 417 | 147.625 | −6.074 | 25.145 | 1.00 | 39.62 | D |
| ATOM | 6920 | CD | GLN D | 417 | 148.512 | −7.253 | 25.464 | 1.00 | 41.04 | D |
| ATOM | 6921 | OE1 | CLN D | 417 | 149.615 | −7.378 | 24.914 | 1.00 | 43.24 | D |
| ATOM | 6922 | NE2 | GLN D | 417 | 148.046 | −8.128 | 26.358 | 1.00 | 40.41 | D |
| ATOM | 6923 | C | GLN D | 417 | 147.506 | −3.532 | 23.450 | 1.00 | 35.61 | D |
| ATOM | 6924 | O | GLN D | 417 | 148.374 | −3.297 | 22.605 | 1.00 | 35.43 | D |
| ATOM | 6925 | N | LEU D | 418 | 147.337 | −2.798 | 24.551 | 1.00 | 35.15 | D |
| ATOM | 6926 | CA | LEU D | 418 | 148.166 | −1.644 | 24.885 | 1.00 | 33.41 | D |
| ATOM | 6927 | CB | LEU D | 418 | 147.315 | −0.523 | 25.429 | 1.00 | 31.33 | D |
| ATOM | 6928 | CG | LEU D | 418 | 146.413 | 0.034 | 24.359 | 1.00 | 31.84 | D |
| ATOM | 6929 | CD1 | LEU D | 418 | 145.508 | 1.119 | 24.943 | 1.00 | 33.02 | D |
| ATOM | 6930 | CD2 | LEU D | 418 | 147.293 | 0.595 | 23.253 | 1.00 | 33.13 | D |
| ATOM | 6931 | C | LEU D | 418 | 149.180 | −2.006 | 25.939 | 1.00 | 33.92 | D |
| ATOM | 6932 | O | LEU D | 418 | 148.811 | −2.281 | 27.076 | 1.00 | 33.63 | D |
| ATOM | 6933 | N | SER D | 419 | 150.458 | −1.991 | 25.572 | 1.00 | 35.50 | D |
| ATOM | 6934 | CA | SER D | 419 | 151.529 | −2.323 | 26.518 | 1.00 | 35.75 | D |
| ATOM | 6935 | CB | SER D | 419 | 152.504 | −3.304 | 25.889 | 1.00 | 36.24 | D |
| ATOM | 6936 | OG | SER D | 419 | 153.366 | −2.611 | 25.006 | 1.00 | 39.10 | D |
| ATOM | 6937 | C | SER D | 419 | 152.286 | −1.060 | 26.903 | 1.00 | 34.65 | D |
| ATOM | 6938 | O | SER D | 419 | 152.148 | −0.036 | 26.243 | 1.00 | 35.71 | D |
| ATOM | 6939 | N | GLY D | 420 | 153.082 | −1.132 | 27.969 | 1.00 | 34.21 | D |
| ATOM | 6940 | CA | GLY D | 420 | 153.845 | 0.030 | 28.407 | 1.00 | 32.81 | D |
| ATOM | 6941 | C | GLY D | 420 | 154.127 | 0.063 | 29.904 | 1.00 | 32.19 | D |
| ATOM | 6942 | O | GLY D | 420 | 155.277 | 0.111 | 30.334 | 1.00 | 32.95 | D |
| ATOM | 6943 | N | HIS D | 421 | 153.057 | 0.038 | 30.690 | 1.00 | 32.53 | D |
| ATOM | 6944 | CA | HIS D | 421 | 153.116 | 0.056 | 32.142 | 1.00 | 31.89 | D |
| ATOM | 6945 | CB | HIS D | 421 | 151.744 | −0.233 | 32.728 | 1.00 | 28.17 | D |
| ATOM | 6946 | CG | HIS D | 421 | 150.862 | 0.972 | 32.856 | 1.00 | 26.59 | D |
| ATOM | 6947 | CD2 | HIS D | 421 | 149.677 | 1.270 | 32.274 | 1.00 | 23.86 | D |
| ATOM | 6948 | ND1 | HIS D | 421 | 151.123 | 1.998 | 33.738 | 1.00 | 24.07 | D |
| ATOM | 6949 | CE1 | HIS D | 421 | 150.133 | 2.872 | 33.699 | 1.00 | 24.39 | D |
| ATOM | 6950 | NE2 | HIS D | 421 | 149.243 | 2.454 | 32.818 | 1.00 | 22.81 | D |
| ATOM | 6951 | C | HIS D | 421 | 154.082 | −0.966 | 32.714 | 1.00 | 34.84 | D |
| ATOM | 6952 | O | HIS D | 421 | 154.005 | −2.162 | 32.399 | 1.00 | 35.95 | D |
| ATOM | 6953 | N | ASP D | 422 | 154.973 | −0.487 | 33.580 | 1.00 | 37.06 | D |
| ATOM | 6954 | CA | ASP D | 422 | 155.969 | −1.328 | 34.241 | 1.00 | 37.22 | D |
| ATOM | 6955 | CB | ASP D | 422 | 157.181 | −0.481 | 34.655 | 1.00 | 37.19 | D |
| ATOM | 6956 | CG | ASP D | 422 | 157.811 | 0.261 | 33.479 | 1.00 | 38.07 | D |
| ATOM | 6957 | OD1 | ASP D | 422 | 158.398 | −0.414 | 32.605 | 1.00 | 36.97 | D |
| ATOM | 6958 | OD2 | ASP D | 422 | 157.716 | 1.519 | 33.426 | 1.00 | 39.00 | D |
| ATOM | 6959 | C | ASP D | 422 | 155.331 | −1.974 | 35.468 | 1.00 | 37.57 | D |
| ATOM | 6960 | O | ASP D | 422 | 155.947 | −2.791 | 36.155 | 1.00 | 38.18 | D |
| ATOM | 6961 | N | GLY D | 423 | 154.089 | −1.584 | 35.735 | 1.00 | 38.03 | D |
| ATOM | 6962 | CA | GLY D | 423 | 153.357 | −2.135 | 36.862 | 1.00 | 37.36 | D |
| ATOM | 6963 | C | GLY D | 423 | 151.963 | −2.599 | 36.451 | 1.00 | 36.40 | D |
| ATOM | 6964 | O | GLY D | 423 | 151.496 | −2.282 | 35.358 | 1.00 | 37.00 | D |
| ATOM | 6965 | N | GLY D | 424 | 151.297 | −3.361 | 37.311 | 1.00 | 35.05 | D |
| ATOM | 6966 | CA | GLY D | 424 | 149.965 | −3.823 | 36.984 | 1.00 | 33.45 | D |
| ATOM | 6967 | C | GLY D | 424 | 149.032 | −2.637 | 36.920 | 1.00 | 33.03 | D |
| ATOM | 6968 | O | GLY D | 424 | 149.183 | −1.692 | 37.687 | 1.00 | 32.43 | D |
| ATOM | 6969 | N | VAL D | 425 | 148.074 | −2.685 | 36.004 | 1.00 | 33.29 | D |
| ATOM | 6970 | CA | VAL D | 425 | 147.112 | −1.602 | 35.826 | 1.00 | 34.26 | D |
| ATOM | 6971 | CB | VAL D | 425 | 146.545 | −1.623 | 34.420 | 1.00 | 34.93 | D |
| ATOM | 6972 | CG1 | VAL D | 425 | 145.785 | −0.328 | 34.150 | 1.00 | 37.13 | D |
| ATOM | 6973 | CG2 | VAL D | 425 | 147.661 | −1.843 | 33.433 | 1.00 | 34.83 | D |
| ATOM | 6974 | C | VAL D | 425 | 145.955 | −1.776 | 36.795 | 1.00 | 33.41 | D |
| ATOM | 6975 | O | VAL D | 425 | 145.473 | −2.880 | 36.975 | 1.00 | 33.61 | D |
| ATOM | 6976 | N | TRP D | 426 | 145.493 | −0.700 | 37.410 | 1.00 | 32.49 | D |
| ATOM | 6977 | CA | TRP D | 426 | 144.395 | −0.837 | 38.343 | 1.00 | 32.03 | D |
| ATOM | 6978 | CB | TRP D | 426 | 144.952 | −0.866 | 39.774 | 1.00 | 30.72 | D |
| ATOM | 6979 | CG | TRP D | 426 | 145.618 | −2.192 | 40.132 | 1.00 | 29.46 | D |
| ATOM | 6980 | CD2 | TRP D | 426 | 145.011 | −3.288 | 40.834 | 1.00 | 27.59 | D |
| ATOM | 6981 | CE2 | TRP D | 426 | 145.982 | −4.311 | 40.937 | 1.00 | 25.67 | D |
| ATOM | 6982 | CE3 | TRP D | 426 | 143.738 | −3.502 | 41.390 | 1.00 | 27.62 | D |
| ATOM | 6983 | CD1 | TRP D | 426 | 146.903 | −2.592 | 39.838 | 1.00 | 28.36 | D |
| ATOM | 6984 | NE1 | TRP D | 426 | 147.122 | −3.863 | 40.321 | 1.00 | 26.42 | D |
| ATOM | 6985 | CZ2 | TRP D | 426 | 145.720 | −5.527 | 41.572 | 1.00 | 23.76 | D |
| ATOM | 6986 | CZ3 | TRP D | 426 | 143.477 | −4.712 | 42.021 | 1.00 | 25.66 | D |
| ATOM | 6987 | CH2 | TRP D | 426 | 144.467 | −5.711 | 42.108 | 1.00 | 24.10 | D |
| ATOM | 6988 | C | TRP D | 426 | 143.291 | 0.224 | 38.190 | 1.00 | 32.39 | D |
| ATOM | 6989 | O | TRP D | 426 | 142.270 | 0.182 | 38.876 | 1.00 | 32.60 | D |
| ATOM | 6990 | N | ALA D | 427 | 143.483 | 1.164 | 37.275 | 1.00 | 31.70 | D |
| ATOM | 6991 | CA | ALA D | 427 | 142.489 | 2.201 | 37.060 | 1.00 | 32.14 | D |
| ATOM | 6992 | CB | ALA D | 427 | 142.699 | 3.330 | 38.059 | 1.00 | 30.77 | D |
| ATOM | 6993 | C | ALA D | 427 | 142.544 | 2.749 | 35.631 | 1.00 | 32.54 | D |
| ATOM | 6994 | O | ALA D | 427 | 143.610 | 3.128 | 35.148 | 1.00 | 33.11 | D |
| ATOM | 6995 | N | LEU D | 428 | 141.403 | 2.763 | 34.949 | 1.00 | 31.96 | D |
| ATOM | 6996 | CA | LEU D | 428 | 141.362 | 3.298 | 33.605 | 1.00 | 31.52 | D |
| ATOM | 6997 | CB | LEU D | 428 | 141.879 | 2.273 | 32.591 | 1.00 | 30.47 | D |
| ATOM | 6998 | CG | LEU D | 428 | 141.523 | 0.793 | 32.619 | 1.00 | 25.70 | D |
| ATOM | 6999 | CD1 | LEU D | 428 | 140.061 | 0.566 | 32.426 | 1.00 | 26.95 | D |
| ATOM | 7000 | CD2 | LEU D | 428 | 142.302 | 0.127 | 31.520 | 1.00 | 26.15 | D |
| ATOM | 7001 | C | LEU D | 428 | 139.998 | 3.823 | 33.185 | 1.00 | 32.41 | D |
| ATOM | 7002 | O | LEU D | 428 | 139.015 | 3.092 | 33.159 | 1.00 | 31.99 | D |
| ATOM | 7003 | N | LYS D | 429 | 139.970 | 5.117 | 32.874 | 1.00 | 34.44 | D |
| ATOM | 7004 | CA | LYS D | 429 | 138.779 | 5.846 | 32.441 | 1.00 | 36.04 | D |
| ATOM | 7005 | CE | LYS D | 429 | 138.655 | 7.149 | 33.247 | 1.00 | 36.79 | D |
| ATOM | 7006 | CG | LYS D | 429 | 137.379 | 7.954 | 33.000 | 1.00 | 41.16 | D |
| ATOM | 7007 | CD | LYS D | 429 | 136.228 | 7.499 | 33.914 | 1.00 | 44.67 | D |
| ATOM | 7008 | CE | LYS D | 429 | 134.828 | 7.936 | 33.399 | 1.00 | 47.43 | D |
| ATOM | 7009 | NZ | LYS D | 429 | 134.385 | 7.223 | 32.133 | 1.00 | 49.62 | D |
| ATOM | 7010 | C | LYS D | 429 | 138.944 | 6.184 | 30.951 | 1.00 | 35.80 | D |
| ATOM | 7011 | O | LYS D | 429 | 139.982 | 6.698 | 30.531 | 1.00 | 35.22 | D |
| ATOM | 7012 | N | TYR D | 430 | 137.926 | 5.888 | 30.152 | 1.00 | 36.71 | D |
| ATOM | 7013 | CA | TYR D | 430 | 137.962 | 6.183 | 28.719 | 1.00 | 38.50 | D |
| ATOM | 7014 | CE | TYR D | 430 | 137.142 | 5.152 | 27.936 | 1.00 | 38.31 | D |
| ATOM | 7015 | CG | TYR D | 430 | 136.840 | 5.560 | 26.501 | 1.00 | 38.68 | D |
| ATOM | 7016 | CD1 | TYR D | 430 | 135.713 | 6.331 | 26.192 | 1.00 | 38.18 | D |
| ATOM | 7017 | CE1 | TYR D | 430 | 135.427 | 6.702 | 24.875 | 1.00 | 37.22 | D |
| ATOM | 7018 | CD2 | TYR D | 430 | 137.680 | 5.173 | 25.449 | 1.00 | 39.59 | D |
| ATOM | 7019 | CE2 | TYR D | 430 | 137.403 | 5.542 | 24.126 | 1.00 | 38.38 | D |
| ATOM | 7020 | CZ | TYR D | 430 | 136.275 | 6.303 | 23.853 | 1.00 | 38.21 | D |
| ATOM | 7021 | OH | TYR D | 430 | 135.983 | 6.646 | 22.557 | 1.00 | 37.82 | D |
| ATOM | 7022 | C | TYR D | 430 | 137.401 | 7.574 | 28.427 | 1.00 | 39.86 | D |
| ATOM | 7023 | O | TYR D | 430 | 136.344 | 7.931 | 28.934 | 1.00 | 39.99 | D |
| ATOM | 7024 | N | ALA D | 431 | 138.109 | 8.349 | 27.607 | 1.00 | 40.69 | D |
| ATOM | 7025 | CA | ALA D | 431 | 137.660 | 9.688 | 27.227 | 1.00 | 41.42 | D |
| ATOM | 7026 | CB | ALA D | 431 | 138.790 | 10.695 | 27.407 | 1.00 | 40.10 | D |
| ATOM | 7027 | C | ALA D | 431 | 137.208 | 9.641 | 25.763 | 1.00 | 43.09 | D |
| ATOM | 7028 | O | ALA D | 431 | 137.842 | 8.998 | 24.916 | 1.00 | 43.69 | D |
| ATOM | 7029 | N | HIS D | 432 | 136.108 | 10.321 | 25.465 | 1.00 | 42.71 | D |
| ATOM | 7030 | CA | HIS D | 432 | 135.584 | 10.340 | 24.102 | 1.00 | 43.16 | D |
| ATOM | 7031 | CB | HIS D | 432 | 134.468 | 11.383 | 23.966 | 1.00 | 47.95 | D |
| ATOM | 7032 | CG | HIS D | 432 | 133.942 | 11.523 | 22.566 | 1.00 | 53.08 | D |
| ATOM | 7033 | CD2 | HIS D | 432 | 133.728 | 12.622 | 21.801 | 1.00 | 53.58 | D |
| ATOM | 7034 | ND1 | HIS D | 432 | 133.594 | 10.439 | 21.787 | 1.00 | 53.70 | D |
| ATOM | 7035 | CE1 | HIS D | 432 | 133.192 | 10.865 | 20.601 | 1.00 | 54.74 | D |
| ATOM | 7036 | NE2 | HIS D | 432 | 133.265 | 12.184 | 20.584 | 1.00 | 54.71 | D |
| ATOM | 7037 | C | HIS D | 432 | 136.646 | 10.636 | 23.052 | 1.00 | 40.32 | D |
| ATOM | 7038 | O | HIS D | 432 | 137.420 | 11.573 | 23.201 | 1.00 | 40.89 | D |
| ATOM | 7039 | N | GLY D | 433 | 136.669 | 9.852 | 21.980 | 1.00 | 37.59 | D |
| ATOM | 7040 | CA | GLY D | 433 | 137.640 | 10.095 | 20.926 | 1.00 | 36.16 | D |
| ATOM | 7041 | C | GLY D | 433 | 138.701 | 9.029 | 20.850 | 1.00 | 34.81 | D |
| ATOM | 7042 | O | GLY D | 433 | 139.697 | 9.177 | 20.143 | 1.00 | 32.92 | D |
| ATOM | 7043 | N | GLY D | 434 | 138.474 | 7.948 | 21.588 | 1.00 | 36.25 | D |
| ATOM | 7044 | CA | GLY D | 434 | 139.412 | 6.841 | 21.617 | 1.00 | 36.22 | D |
| ATOM | 7045 | C | GLY D | 434 | 140.580 | 7.079 | 22.557 | 1.00 | 36.76 | D |
| ATOM | 7046 | O | GLY D | 434 | 141.559 | 6.338 | 22.493 | 1.00 | 37.12 | D |
| ATOM | 7047 | N | ILE D | 435 | 140.469 | 8.106 | 23.411 | 1.00 | 37.42 | D |
| ATOM | 7048 | CA | ILE D | 435 | 141.501 | 8.478 | 24.397 | 1.00 | 37.95 | D |
| ATOM | 7049 | CB | ILE D | 435 | 141.424 | 9.992 | 24.784 | 1.00 | 39.16 | D |
| ATOM | 7050 | CG2 | ILE D | 435 | 142.684 | 10.430 | 25.539 | 1.00 | 37.49 | D |
| ATOM | 7051 | CG1 | ILE D | 435 | 141.328 | 10.839 | 23.519 | 1.00 | 39.15 | D |
| ATOM | 7052 | CD1 | ILE D | 435 | 142.476 | 10.580 | 22.572 | 1.00 | 38.06 | D |
| ATOM | 7053 | C | ILE D | 435 | 141.273 | 7.662 | 25.658 | 1.00 | 37.16 | D |
| ATOM | 7054 | O | ILE D | 435 | 140.130 | 7.426 | 26.059 | 1.00 | 36.94 | D |
| ATOM | 7055 | N | LEU D | 436 | 142.360 | 7.251 | 26.297 | 1.00 | 35.58 | D |
| ATOM | 7056 | CA | LEU D | 436 | 142.243 | 6.427 | 27.481 | 1.00 | 34.59 | D |
| ATOM | 7057 | CB | LEU D | 436 | 142.354 | 4.980 | 27.046 | 1.00 | 35.11 | D |
| ATOM | 7058 | CG | LEU D | 436 | 141.894 | 3.866 | 27.965 | 1.00 | 35.32 | D |
| ATOM | 7059 | CD1 | LEU D | 436 | 142.000 | 2.560 | 27.178 | 1.00 | 34.07 | D |
| ATOM | 7060 | CD2 | LEU D | 436 | 142.727 | 3.819 | 29.234 | 1.00 | 35.01 | D |
| ATOM | 7061 | C | LEU D | 436 | 143.309 | 6.742 | 28.511 | 1.00 | 34.64 | D |
| ATOM | 7062 | O | LEU D | 436 | 144.495 | 6.786 | 28.195 | 1.00 | 35.84 | D |
| ATOM | 7063 | N | VAL D | 437 | 142.891 | 6.960 | 29.750 | 1.00 | 33.63 | D |
| ATOM | 7064 | CA | VAL D | 437 | 143.854 | 7.244 | 30.801 | 1.00 | 32.09 | D |
| ATOM | 7065 | CB | VAL D | 437 | 143.484 | 8.519 | 31.575 | 1.00 | 31.66 | D |
| ATOM | 7066 | CG1 | VAL D | 437 | 144.624 | 8.902 | 32.506 | 1.00 | 32.49 | D |
| ATOM | 7067 | CG2 | VAL D | 437 | 143.172 | 9.650 | 30.608 | 1.00 | 31.31 | D |
| ATOM | 7068 | C | VAL D | 437 | 143.887 | 6.058 | 31.764 | 1.00 | 31.94 | D |
| ATOM | 7069 | O | VAL D | 437 | 142.845 | 5.454 | 32.047 | 1.00 | 31.55 | D |
| ATOM | 7070 | N | SER D | 438 | 145.080 | 5.707 | 32.246 | 1.00 | 29.85 | D |
| ATOM | 7071 | CA | SER D | 438 | 145.201 | 4.597 | 33.189 | 1.00 | 26.33 | D |
| ATOM | 7072 | CB | SER D | 438 | 145.601 | 3.302 | 32.480 | 1.00 | 25.65 | D |
| ATOM | 7073 | OG | SER D | 438 | 146.858 | 3.426 | 31.852 | 1.00 | 26.66 | D |
| ATOM | 7074 | C | SER D | 438 | 146.156 | 4.853 | 34.336 | 1.00 | 23.90 | D |
| ATOM | 7075 | O | SER D | 438 | 146.977 | 5.765 | 34.294 | 1.00 | 21.89 | D |
| ATOM | 7076 | N | GLY D | 439 | 146.019 | 4.025 | 35.366 | 1.00 | 23.29 | D |
| ATOM | 7077 | CA | GLY D | 439 | 146.835 | 4.130 | 36.563 | 1.00 | 23.55 | D |
| ATOM | 7078 | C | GLY D | 439 | 147.396 | 2.783 | 36.962 | 1.00 | 23.12 | D |
| ATOM | 7079 | O | GLY D | 439 | 146.725 | 1.748 | 36.883 | 1.00 | 22.22 | D |
| ATOM | 7080 | N | SER D | 440 | 148.634 | 2.800 | 37.429 | 1.00 | 23.85 | D |
| ATOM | 7081 | CA | SER D | 440 | 149.302 | 1.562 | 37.786 | 1.00 | 25.71 | D |
| ATOM | 7082 | CB | SER D | 440 | 150.244 | 1.169 | 36.630 | 1.00 | 24.70 | D |
| ATOM | 7083 | OG | SER D | 440 | 151.145 | 0.129 | 36.979 | 1.00 | 23.47 | D |
| ATOM | 7084 | C | SER D | 440 | 150.078 | 1.620 | 39.099 | 1.00 | 26.18 | D |
| ATOM | 7085 | O | SER D | 440 | 150.112 | 2.651 | 39.793 | 1.00 | 26.21 | D |
| ATOM | 7086 | N | THR D | 441 | 150.695 | 0.481 | 39.413 | 1.00 | 27.38 | D |
| ATOM | 7087 | CA | THR D | 441 | 151.521 | 0.308 | 40.593 | 1.00 | 27.39 | D |
| ATOM | 7088 | CB | THR D | 441 | 151.676 | −1.165 | 40.968 | 1.00 | 28.12 | D |
| ATOM | 7089 | OG1 | THR D | 441 | 152.519 | −1.810 | 40.004 | 1.00 | 27.63 | D |
| ATOM | 7090 | CG2 | THR D | 441 | 150.310 | −1.858 | 41.003 | 1.00 | 28.73 | D |
| ATOM | 7091 | C | THR D | 441 | 152.903 | 0.847 | 40.237 | 1.00 | 28.31 | D |
| ATOM | 7092 | O | THR D | 441 | 153.768 | 0.906 | 41.090 | 1.00 | 29.00 | D |
| ATOM | 7093 | N | ASP D | 442 | 153.116 | 1.234 | 38.980 | 1.00 | 29.46 | D |
| ATOM | 7094 | CA | ASP D | 442 | 154.402 | 1.796 | 38.603 | 1.00 | 31.38 | D |
| ATOM | 7095 | CB | ASP D | 442 | 154.724 | 1.551 | 37.126 | 1.00 | 33.65 | D |
| ATOM | 7096 | CG | ASP D | 442 | 153.695 | 2.167 | 36.185 | 1.00 | 35.87 | D |
| ATOM | 7097 | OD1 | ASP D | 442 | 152.845 | 2.962 | 36.680 | 1.00 | 35.07 | D |
| ATOM | 7098 | OD2 | ASP D | 442 | 153.753 | 1.860 | 34.959 | 1.00 | 34.85 | D |
| ATOM | 7099 | C | ASP D | 442 | 154.351 | 3.287 | 38.857 | 1.00 | 31.58 | D |
| ATOM | 7100 | O | ASP D | 442 | 155.161 | 4.049 | 38.334 | 1.00 | 31.31 | D |
| ATOM | 7101 | N | ARG D | 443 | 153.360 | 3.700 | 39.635 | 1.00 | 31.79 | D |
| ATOM | 7102 | CA | ARG D | 443 | 153.191 | 5.100 | 39.983 | 1.00 | 31.82 | D |
| ATOM | 7103 | CB | ARG D | 443 | 154.413 | 5.569 | 40.760 | 1.00 | 28.96 | D |
| ATOM | 7104 | CG | ARG D | 443 | 154.805 | 4.604 | 41.846 | 1.00 | 26.68 | D |
| ATOM | 7105 | CD | ARG D | 443 | 156.056 | 5.065 | 42.541 | 1.00 | 25.25 | D |
| ATOM | 7106 | NE | ARG D | 443 | 156.496 | 4.111 | 43.541 | 1.00 | 26.33 | D |
| ATOM | 7107 | CZ | ARG D | 443 | 157.552 | 4.303 | 44.319 | 1.00 | 27.22 | D |
| ATOM | 7108 | NH1 | ARG D | 443 | 158.250 | 5.419 | 44.192 | 1.00 | 28.58 | D |
| ATOM | 7109 | NH2 | ARG D | 443 | 157.916 | 3.388 | 45.213 | 1.00 | 26.32 | D |
| ATOM | 7110 | C | ARG D | 443 | 152.955 | 6.016 | 38.783 | 1.00 | 32.05 | D |
| ATOM | 7111 | O | ARG D | 443 | 152.975 | 7.243 | 38.917 | 1.00 | 32.48 | D |
| ATOM | 7112 | N | THR D | 444 | 152.705 | 5.439 | 37.616 | 1.00 | 31.82 | D |
| ATOM | 7113 | CA | THR D | 444 | 152.486 | 6.283 | 36.459 | 1.00 | 32.99 | D |
| ATOM | 7114 | CB | THR D | 444 | 153.335 | 5.824 | 35.246 | 1.00 | 32.98 | D |
| ATOM | 7115 | OG1 | THR D | 444 | 152.768 | 4.643 | 34.657 | 1.00 | 30.88 | D |
| ATOM | 7116 | CG2 | THR D | 444 | 154.757 | 5.549 | 35.680 | 1.00 | 31.91 | D |
| ATOM | 7117 | C | THR D | 444 | 151.036 | 6.451 | 36.000 | 1.00 | 34.38 | D |
| ATOM | 7118 | O | THR D | 444 | 150.182 | 5.570 | 36.164 | 1.00 | 34.02 | D |
| ATOM | 7119 | N | VAL D | 445 | 150.784 | 7.623 | 35.432 | 1.00 | 35.02 | D |
| ATOM | 7120 | CA | VAL D | 445 | 149.492 | 7.992 | 34.888 | 1.00 | 35.88 | D |
| ATOM | 7121 | CB | VAL D | 445 | 149.049 | 9.384 | 35.406 | 1.00 | 34.38 | D |
| ATOM | 7122 | CG1 | VAL D | 445 | 147.839 | 9.871 | 34.640 | 1.00 | 33.91 | D |
| ATOM | 7123 | CG2 | VAL D | 445 | 148.747 | 9.312 | 36.893 | 1.00 | 33.31 | D |
| ATOM | 7124 | C | VAL D | 445 | 149.787 | 8.067 | 33.392 | 1.00 | 38.00 | D |
| ATOM | 7125 | O | VAL D | 445 | 150.648 | 8.841 | 32.969 | 1.00 | 40.09 | D |
| ATOM | 7126 | N | ARG D | 446 | 149.116 | 7.256 | 32.583 | 1.00 | 38.70 | D |
| ATOM | 7127 | CA | ARG D | 446 | 149.390 | 7.299 | 31.152 | 1.00 | 40.25 | D |
| ATOM | 7128 | CB | ARG D | 446 | 150.102 | 6.016 | 30.707 | 1.00 | 40.97 | D |
| ATOM | 7129 | CG | ARG D | 446 | 151.387 | 5.796 | 31.448 | 1.00 | 42.38 | D |
| ATOM | 7130 | CD | ARG D | 446 | 152.190 | 4.636 | 30.925 | 1.00 | 44.42 | D |
| ATOM | 7131 | NE | ARG D | 446 | 153.302 | 4.360 | 31.835 | 1.00 | 48.85 | D |
| ATOM | 7132 | CZ | ARG D | 446 | 154.291 | 3.506 | 31.588 | 1.00 | 50.95 | D |
| ATOM | 7133 | NH1 | ARG D | 446 | 154.316 | 2.834 | 30.442 | 1.00 | 52.13 | D |
| ATOM | 7134 | NH2 | ARG D | 446 | 155.251 | 3.320 | 32.490 | 1.00 | 51.46 | D |
| ATOM | 7135 | C | ARG D | 446 | 148.155 | 7.527 | 30.296 | 1.00 | 40.71 | D |
| ATOM | 7136 | O | ARG D | 446 | 147.050 | 7.083 | 30.635 | 1.00 | 39.57 | D |
| ATOM | 7137 | N | VAL D | 447 | 148.357 | 8.253 | 29.195 | 1.00 | 40.90 | D |
| ATOM | 7138 | CA | VAL D | 447 | 147.293 | 8.552 | 28.237 | 1.00 | 41.54 | D |
| ATOM | 7139 | CB | VAL D | 447 | 147.260 | 10.037 | 27.849 | 1.00 | 40.47 | D |
| ATOM | 7140 | CG1 | VAL D | 447 | 146.043 | 10.306 | 26.981 | 1.00 | 38.69 | D |
| ATOM | 7141 | CG2 | VAL D | 447 | 147.226 | 10.895 | 29.100 | 1.00 | 40.93 | D |
| ATOM | 7142 | C | VAL D | 447 | 147.566 | 7.735 | 26.991 | 1.00 | 42.31 | D |
| ATOM | 7143 | O | VAL D | 447 | 148.689 | 7.712 | 26.501 | 1.00 | 43.98 | D |
| ATOM | 7144 | N | TRP D | 448 | 146.549 | 7.055 | 26.481 | 1.00 | 42.01 | D |
| ATOM | 7145 | CA | TRP D | 448 | 146.730 | 6.234 | 25.296 | 1.00 | 42.06 | D |
| ATOM | 7146 | CB | TRP D | 448 | 146.416 | 4.769 | 25.625 | 1.00 | 40.93 | D |
| ATOM | 7147 | CG | TRP D | 448 | 147.108 | 4.254 | 26.849 | 1.00 | 40.06 | D |
| ATOM | 7148 | CD2 | TRP D | 448 | 148.167 | 3.294 | 26.890 | 1.00 | 39.39 | D |
| ATOM | 7149 | CE2 | TRP D | 448 | 148.529 | 3.127 | 28.247 | 1.00 | 39.74 | D |
| ATOM | 7150 | CE3 | TRP D | 448 | 148.846 | 2.555 | 25.911 | 1.00 | 37.93 | D |
| ATOM | 7151 | CD1 | TRP D | 448 | 146.871 | 4.621 | 28.145 | 1.00 | 39.85 | D |
| ATOM | 7152 | NE1 | TRP D | 448 | 147.720 | 3.949 | 28.992 | 1.00 | 39.87 | D |
| ATOM | 7153 | CZ2 | TRP D | 448 | 149.552 | 2.247 | 28.653 | 1.00 | 38.84 | D |
| ATOM | 7154 | CZ3 | TRP D | 448 | 149.860 | 1.680 | 26.314 | 1.00 | 38.08 | D |
| ATOM | 7155 | CH2 | TRP D | 448 | 150.203 | 1.537 | 27.679 | 1.00 | 37.48 | D |
| ATOM | 7156 | C | TRP D | 448 | 145.825 | 6.705 | 24.156 | 1.00 | 42.74 | D |
| ATOM | 7157 | O | TRP D | 448 | 144.933 | 7.537 | 24.349 | 1.00 | 42.62 | D |
| ATOM | 7158 | N | ASP D | 449 | 146.067 | 6.165 | 22.967 | 1.00 | 43.66 | D |
| ATOM | 7159 | CA | ASP D | 449 | 145.265 | 6.485 | 21.797 | 1.00 | 44.36 | D |
| ATOM | 7160 | CB | ASP D | 449 | 146.062 | 7.368 | 20.836 | 1.00 | 45.33 | D |
| ATOM | 7161 | CG | ASP D | 449 | 145.256 | 7.782 | 19.616 | 1.00 | 46.25 | D |
| ATOM | 7162 | OD1 | ASP D | 449 | 145.791 | 8.522 | 18.771 | 1.00 | 46.26 | D |
| ATOM | 7163 | OD2 | ASP D | 449 | 144.088 | 7.370 | 19.494 | 1.00 | 47.22 | D |
| ATOM | 7164 | C | ASP D | 449 | 144.899 | 5.169 | 21.119 | 1.00 | 44.82 | D |
| ATOM | 7165 | O | ASP D | 449 | 145.735 | 4.551 | 20.468 | 1.00 | 44.81 | D |
| ATOM | 7166 | N | ILE D | 450 | 143.653 | 4.740 | 21.280 | 1.00 | 45.73 | D |
| ATOM | 7167 | CA | ILE D | 450 | 143.205 | 3.484 | 20.687 | 1.00 | 47.64 | D |
| ATOM | 7168 | CB | ILE D | 450 | 141.711 | 3.215 | 21.000 | 1.00 | 46.91 | D |
| ATOM | 7169 | CG2 | ILE D | 450 | 141.335 | 1.820 | 20.527 | 1.00 | 45.80 | D |
| ATOM | 7170 | CG1 | ILE D | 450 | 141.455 | 3.357 | 22.507 | 1.00 | 46.32 | D |
| ATOM | 7171 | CD1 | ILE D | 450 | 140.009 | 3.138 | 22.920 | 1.00 | 46.07 | D |
| ATOM | 7172 | C | ILE D | 450 | 143.395 | 3.516 | 19.173 | 1.00 | 49.71 | D |
| ATOM | 7173 | O | ILE D | 450 | 143.890 | 2.556 | 18.573 | 1.00 | 49.74 | D |
| ATOM | 7174 | N | LYS D | 451 | 142.984 | 4.631 | 18.571 | 1.00 | 51.95 | D |
| ATOM | 7175 | CA | LYS D | 451 | 143.092 | 4.859 | 17.130 | 1.00 | 52.66 | D |
| ATOM | 7176 | CB | LYS D | 451 | 142.789 | 6.329 | 16.815 | 1.00 | 54.03 | D |
| ATOM | 7177 | CG | LYS D | 451 | 141.425 | 6.825 | 17.283 | 1.00 | 54.77 | D |
| ATOM | 7178 | CD | LYS D | 451 | 140.315 | 6.348 | 16.362 | 1.00 | 56.08 | D |
| ATOM | 7179 | CE | LYS D | 451 | 138.958 | 6.853 | 16.829 | 1.00 | 58.40 | D |
| ATOM | 7180 | NZ | LYS D | 451 | 138.564 | 6.293 | 18.170 | 1.00 | 60.81 | D |
| ATOM | 7181 | C | LYS D | 451 | 144.519 | 4.534 | 16.687 | 1.00 | 53.12 | D |
| ATOM | 7182 | O | LYS D | 451 | 144.743 | 3.637 | 15.875 | 1.00 | 52.92 | D |
| ATOM | 7183 | N | LYS D | 452 | 145.478 | 5.279 | 17.236 | 1.00 | 53.06 | D |
| ATOM | 7184 | CA | LYS D | 452 | 146.889 | 5.081 | 16.922 | 1.00 | 53.16 | D |
| ATOM | 7185 | CB | LYS D | 452 | 147.727 | 6.274 | 17.405 | 1.00 | 54.65 | D |
| ATOM | 7186 | CG | LYS D | 452 | 147.432 | 7.608 | 16.705 | 1.00 | 55.78 | D |
| ATOM | 7187 | CD | LYS D | 452 | 148.434 | 8.689 | 17.106 | 1.00 | 54.91 | D |
| ATOM | 7188 | CE | LYS D | 452 | 148.269 | 9.952 | 16.262 | 1.00 | 56.24 | D |
| ATOM | 7189 | NZ | LYS D | 452 | 146.931 | 10.592 | 16.394 | 1.00 | 55.58 | D |
| ATOM | 7190 | C | LYS D | 452 | 147.426 | 3.801 | 17.565 | 1.00 | 52.16 | D |
| ATOM | 7191 | O | LYS D | 452 | 148.530 | 3.352 | 17.257 | 1.00 | 52.14 | D |
| ATOM | 7192 | N | GLY D | 453 | 146.646 | 3.221 | 18.466 | 1.00 | 51.03 | D |
| ATOM | 7193 | CA | GLY D | 453 | 147.070 | 2.000 | 19.119 | 1.00 | 48.98 | D |
| ATOM | 7194 | C | GLY D | 453 | 148.425 | 2.076 | 19.795 | 1.00 | 47.55 | D |
| ATOM | 7195 | O | GLY D | 453 | 149.268 | 1.206 | 19.598 | 1.00 | 48.41 | D |
| ATOM | 7196 | N | CYS D | 454 | 148.643 | 3.111 | 20.595 | 1.00 | 45.89 | D |
| ATOM | 7197 | CA | CYS D | 454 | 149.903 | 3.252 | 21.306 | 1.00 | 45.10 | D |
| ATOM | 7198 | CB | CYS D | 454 | 151.007 | 3.764 | 20.371 | 1.00 | 45.35 | D |
| ATOM | 7199 | SG | CYS D | 454 | 151.016 | 5.592 | 20.180 | 1.00 | 46.05 | D |
| ATOM | 7200 | C | CYS D | 454 | 149.733 | 4.246 | 22.445 | 1.00 | 45.09 | D |
| ATOM | 7201 | O | CYS D | 454 | 148.660 | 4.827 | 22.632 | 1.00 | 43.95 | D |
| ATOM | 7202 | N | CYS D | 455 | 150.815 | 4.443 | 23.191 | 1.00 | 45.72 | D |
| ATOM | 7203 | CA | CYS D | 455 | 150.831 | 5.373 | 24.307 | 1.00 | 46.22 | D |
| ATOM | 7204 | CB | CYS D | 455 | 151.663 | 4.818 | 25.461 | 1.00 | 46.14 | D |
| ATOM | 7205 | SG | CYS D | 455 | 151.652 | 5.917 | 26.902 | 1.00 | 44.91 | D |
| ATOM | 7206 | C | CYS D | 455 | 151.412 | 6.717 | 23.880 | 1.00 | 45.90 | D |
| ATOM | 7207 | O | CYS D | 455 | 152.504 | 6.787 | 23.328 | 1.00 | 45.72 | D |
| ATOM | 7208 | N | THR D | 456 | 150.674 | 7.778 | 24.160 | 1.00 | 46.41 | D |
| ATOM | 7209 | CA | THR D | 456 | 151.089 | 9.114 | 23.807 | 1.00 | 47.30 | D |
| ATOM | 7210 | CB | THR D | 456 | 149.878 | 9.947 | 23.316 | 1.00 | 47.67 | D |
| ATOM | 7211 | OG1 | THR D | 456 | 150.170 | 11.344 | 23.454 | 1.00 | 49.91 | D |
| ATOM | 7212 | CG2 | THR D | 456 | 148.633 | 9.623 | 24.124 | 1.00 | 48.32 | D |
| ATOM | 7213 | C | THR D | 456 | 151.767 | 9.870 | 24.953 | 1.00 | 47.34 | D |
| ATOM | 7214 | O | THR D | 456 | 152.722 | 10.614 | 24.729 | 1.00 | 47.57 | D |
| ATOM | 7215 | N | HIS D | 457 | 151.280 | 9.691 | 26.177 | 1.00 | 46.90 | D |
| ATOM | 7216 | CA | HIS D | 457 | 151.850 | 10.407 | 27.315 | 1.00 | 46.66 | D |
| ATOM | 7217 | CB | HIS D | 457 | 150.935 | 11.569 | 27.708 | 1.00 | 48.78 | D |
| ATOM | 7218 | CG | HIS D | 457 | 150.781 | 12.608 | 26.639 | 1.00 | 52.07 | D |
| ATOM | 7219 | CD2 | HIS D | 457 | 149.752 | 12.884 | 25.802 | 1.00 | 53.00 | D |
| ATOM | 7220 | ND1 | HIS D | 457 | 151.768 | 13.525 | 26.349 | 1.00 | 51.90 | D |
| ATOM | 7221 | CE1 | HIS D | 457 | 151.351 | 14.324 | 25.383 | 1.00 | 53.55 | D |
| ATOM | 7222 | NE2 | HIS D | 457 | 150.131 | 13.957 | 25.033 | 1.00 | 53.69 | D |
| ATOM | 7223 | C | HIS D | 457 | 152.101 | 9.534 | 28.529 | 1.00 | 45.09 | D |
| ATOM | 7224 | O | HIS D | 457 | 151.360 | 8.591 | 28.797 | 1.00 | 46.21 | D |
| ATOM | 7225 | N | VAL D | 458 | 153.163 | 9.864 | 29.255 | 1.00 | 43.08 | D |
| ATOM | 7226 | CA | VAL D | 458 | 153.544 | 9.142 | 30.462 | 1.00 | 40.12 | D |
| ATOM | 7227 | CB | VAL D | 458 | 154.847 | 8.350 | 30.247 | 1.00 | 38.48 | D |
| ATOM | 7228 | CG1 | VAL D | 458 | 155.106 | 7.437 | 31.430 | 1.00 | 36.35 | D |
| ATOM | 7229 | CG2 | VAL D | 458 | 154.758 | 7.554 | 28.954 | 1.00 | 37.80 | D |
| ATOM | 7230 | C | VAL D | 458 | 153.761 | 10.197 | 31.535 | 1.00 | 38.68 | D |
| ATOM | 7231 | O | VAL D | 458 | 154.733 | 10.942 | 31.474 | 1.00 | 38.06 | D |
| ATOM | 7232 | N | PHE D | 459 | 152.834 | 10.267 | 32.491 | 1.00 | 37.03 | D |
| ATOM | 7233 | CA | PHE D | 459 | 152.902 | 11.230 | 33.588 | 1.00 | 35.98 | D |
| ATOM | 7234 | CB | PHE D | 459 | 151.526 | 11.848 | 33.859 | 1.00 | 35.38 | D |
| ATOM | 7235 | CG | PHE D | 459 | 151.009 | 12.705 | 32.729 | 1.00 | 36.52 | D |
| ATOM | 7236 | CD1 | PHE D | 459 | 149.643 | 12.975 | 32.603 | 1.00 | 35.53 | D |
| ATOM | 7237 | CD2 | PHE D | 459 | 151.882 | 13.228 | 31.773 | 1.00 | 36.02 | D |
| ATOM | 7238 | CE1 | PHE D | 459 | 149.160 | 13.748 | 31.542 | 1.00 | 33.32 | D |
| ATOM | 7239 | CE2 | PHE D | 459 | 151.404 | 13.999 | 30.715 | 1.00 | 34.61 | D |
| ATOM | 7240 | CZ | PHE D | 459 | 150.040 | 14.258 | 30.599 | 1.00 | 33.14 | D |
| ATOM | 7241 | C | PHE D | 459 | 153.421 | 10.583 | 34.858 | 1.00 | 35.63 | D |
| ATOM | 7242 | O | PHE D | 459 | 152.927 | 9.540 | 35.286 | 1.00 | 36.76 | D |
| ATOM | 7243 | N | GLU D | 460 | 154.436 | 11.210 | 35.443 | 1.00 | 34.40 | D |
| ATOM | 7244 | CA | GLU D | 460 | 155.042 | 10.731 | 36.669 | 1.00 | 32.85 | D |
| ATOM | 7245 | CB | GLD D | 460 | 156.541 | 10.572 | 36.501 | 1.00 | 31.50 | D |
| ATOM | 7246 | CG | GLU D | 460 | 156.912 | 9.591 | 35.436 | 1.00 | 34.06 | D |
| ATOM | 7247 | CD | GLU D | 460 | 158.340 | 9.146 | 35.561 | 1.00 | 34.99 | D |
| ATOM | 7248 | OE1 | GLU D | 460 | 159.177 | 9.996 | 35.916 | 1.00 | 38.06 | D |
| ATOM | 7249 | OE2 | GLU D | 460 | 158.634 | 7.959 | 35.305 | 1.00 | 34.49 | D |
| ATOM | 7250 | C | GLU D | 460 | 154.754 | 11.770 | 37.717 | 1.00 | 32.81 | D |
| ATOM | 7251 | O | GLU D | 460 | 154.506 | 12.927 | 37.387 | 1.00 | 33.34 | D |
| ATOM | 7252 | N | GLY D | 461 | 154.788 | 11.364 | 38.982 | 1.00 | 33.03 | D |
| ATOM | 7253 | CA | GLY D | 461 | 154.503 | 12.295 | 40.057 | 1.00 | 31.23 | D |
| ATOM | 7254 | C | GLY D | 461 | 154.150 | 11.600 | 41.354 | 1.00 | 30.79 | D |
| ATOM | 7255 | O | GLY D | 461 | 154.770 | 11.857 | 42.383 | 1.00 | 31.55 | D |
| ATOM | 7256 | N | HIS D | 462 | 153.151 | 10.727 | 41.318 | 1.00 | 30.63 | D |
| ATOM | 7257 | CA | HIS D | 462 | 152.744 | 10.016 | 42.522 | 1.00 | 30.77 | D |
| ATOM | 7258 | CB | HIS D | 462 | 151.580 | 9.065 | 42.209 | 1.00 | 30.12 | D |
| ATOM | 7259 | CG | HIS D | 462 | 150.254 | 9.754 | 42.066 | 1.00 | 29.59 | D |
| ATOM | 7260 | CD2 | HIS D | 462 | 149.387 | 9.814 | 41.026 | 1.00 | 29.20 | D |
| ATOM | 7261 | ND1 | HIS D | 462 | 149.701 | 10.520 | 43.070 | 1.00 | 27.98 | D |
| ATOM | 7262 | CE1 | HIS D | 462 | 148.555 | 11.027 | 42.652 | 1.00 | 29.48 | D |
| ATOM | 7263 | NE2 | HIS D | 462 | 148.342 | 10.615 | 41.415 | 1.00 | 29.45 | D |
| ATOM | 7264 | C | HIS D | 462 | 153.935 | 9.233 | 43.032 | 1.00 | 31.13 | D |
| ATOM | 7265 | O | HIS D | 462 | 154.687 | 8.672 | 42.240 | 1.00 | 32.76 | D |
| ATOM | 7266 | N | ASN D | 463 | 154.113 | 9.195 | 44.349 | 1.00 | 31.08 | D |
| ATOM | 7267 | CA | ASN D | 463 | 155.239 | 8.473 | 44.929 | 1.00 | 30.79 | D |
| ATOM | 7268 | CB | ASN D | 463 | 155.764 | 9.212 | 46.155 | 1.00 | 31.33 | D |
| ATOM | 7269 | CG | ASN D | 463 | 156.351 | 10.572 | 45.806 | 1.00 | 33.80 | D |
| ATOM | 7270 | OD1 | ASN D | 463 | 156.669 | 11.368 | 46.700 | 1.00 | 36.56 | D |
| ATOM | 7271 | ND2 | ASN D | 463 | 156.500 | 10.848 | 44.511 | 1.00 | 31.92 | D |
| ATOM | 7272 | C | ASN D | 463 | 154.863 | 7.058 | 45.300 | 1.00 | 30.83 | D |
| ATOM | 7273 | O | ASN D | 463 | 155.678 | 6.319 | 45.843 | 1.00 | 32.01 | D |
| ATOM | 7274 | N | SER D | 464 | 153.623 | 6.685 | 44.996 | 1.00 | 30.09 | D |
| ATOM | 7275 | CA | SER D | 464 | 153.099 | 5.346 | 45.292 | 1.00 | 29.60 | D |
| ATOM | 7276 | CB | SER D | 464 | 152.364 | 5.358 | 46.630 | 1.00 | 31.07 | D |
| ATOM | 7277 | OG | SER D | 464 | 153.073 | 4.621 | 47.611 | 1.00 | 34.87 | D |
| ATOM | 7278 | C | SER D | 464 | 152.125 | 4.900 | 44.203 | 1.00 | 28.55 | D |
| ATOM | 7279 | O | SER D | 464 | 151.814 | 5.656 | 43.289 | 1.00 | 29.29 | D |
| ATOM | 7280 | N | THR D | 465 | 151.625 | 3.676 | 44.313 | 1.00 | 27.12 | D |
| ATOM | 7281 | CA | THR D | 465 | 150.675 | 3.153 | 43.331 | 1.00 | 25.67 | D |
| ATOM | 7282 | CB | THR D | 465 | 150.036 | 1.828 | 43.817 | 1.00 | 23.85 | D |
| ATOM | 7283 | OG1 | THR D | 465 | 151.057 | 0.894 | 44.197 | 1.00 | 24.69 | D |
| ATOM | 7284 | CG2 | THR D | 465 | 149.169 | 1.234 | 42.742 | 1.00 | 22.12 | D |
| ATOM | 7285 | C | THR D | 465 | 149.536 | 4.150 | 43.089 | 1.00 | 26.17 | D |
| ATOM | 7286 | O | THR D | 465 | 149.122 | 4.872 | 44.001 | 1.00 | 27.21 | D |
| ATOM | 7287 | N | VAL D | 466 | 149.041 | 4.197 | 41.856 | 1.00 | 25.63 | D |
| ATOM | 7288 | CA | VAL D | 466 | 147.933 | 5.078 | 41.506 | 1.00 | 24.45 | D |
| ATOM | 7289 | CB | VAL D | 466 | 148.003 | 5.484 | 39.994 | 1.00 | 26.90 | D |
| ATOM | 7290 | CG1 | VAL D | 466 | 146.795 | 6.351 | 39.597 | 1.00 | 27.11 | D |
| ATOM | 7291 | CG2 | VAL D | 466 | 149.300 | 6.240 | 39.732 | 1.00 | 27.75 | D |
| ATOM | 7292 | C | VAL D | 466 | 146.740 | 4.165 | 41.783 | 1.00 | 22.49 | D |
| ATOM | 7293 | O | VAL D | 466 | 146.604 | 3.130 | 41.148 | 1.00 | 19.72 | D |
| ATOM | 7294 | N | ARG D | 467 | 145.894 | 4.543 | 42.737 | 1.00 | 21.80 | D |
| ATOM | 7295 | CA | ARG D | 467 | 144.764 | 3.713 | 43.121 | 1.00 | 21.09 | D |
| ATOM | 7296 | CB | ARG D | 467 | 144.529 | 3.828 | 44.637 | 1.00 | 21.63 | D |
| ATOM | 7297 | CG | ARG D | 467 | 143.582 | 2.778 | 45.221 | 1.00 | 24.03 | D |
| ATOM | 7298 | CD | ARG D | 467 | 144.190 | 1.398 | 45.033 | 1.00 | 25.89 | D |
| ATOM | 7299 | NE | ARG D | 467 | 143.247 | 0.294 | 45.158 | 1.00 | 27.91 | D |
| ATOM | 7300 | CZ | ARG D | 467 | 142.283 | 0.013 | 44.288 | 1.00 | 30.10 | D |
| ATOM | 7301 | NH1 | ARG D | 467 | 142.117 | 0.776 | 43.214 | 1.00 | 32.22 | D |
| ATOM | 7302 | NH2 | ARG D | 467 | 141.515 | −1.064 | 44.469 | 1.00 | 28.51 | D |
| ATOM | 7303 | C | ARG D | 467 | 143.463 | 3.984 | 42.370 | 1.00 | 21.53 | D |
| ATOM | 7304 | O | ARG D | 467 | 142.666 | 3.071 | 42.152 | 1.00 | 21.18 | D |
| ATOM | 7305 | N | CYS D | 468 | 143.240 | 5.225 | 41.960 | 1.00 | 21.08 | D |
| ATOM | 7306 | CA | CYS D | 468 | 142.008 | 5.538 | 41.259 | 1.00 | 22.04 | D |
| ATOM | 7307 | CB | CYS D | 468 | 140.874 | 5.672 | 42.282 | 1.00 | 22.73 | D |
| ATOM | 7308 | SG | CYS D | 468 | 141.036 | 7.052 | 43.480 | 1.00 | 26.78 | D |
| ATOM | 7309 | C | CYS D | 468 | 142.141 | 6.807 | 40.433 | 1.00 | 22.89 | D |
| ATOM | 7310 | O | CYS D | 468 | 143.034 | 7.617 | 40.672 | 1.00 | 24.06 | D |
| ATOM | 7311 | N | LEU D | 469 | 141.247 | 6.995 | 39.466 | 1.00 | 23.65 | D |
| ATOM | 7312 | CA | LEU D | 469 | 141.321 | 8.189 | 38.614 | 1.00 | 25.22 | D |
| ATOM | 7313 | CB | LEU D | 469 | 142.382 | 7.997 | 37.512 | 1.00 | 25.67 | D |
| ATOM | 7314 | CG | LEU D | 469 | 142.191 | 6.914 | 36.441 | 1.00 | 25.46 | D |
| ATOM | 7315 | CD1 | LEU D | 469 | 140.965 | 7.170 | 35.605 | 1.00 | 24.47 | D |
| ATOM | 7316 | CD2 | LEU D | 469 | 143.428 | 6.891 | 35.560 | 1.00 | 26.81 | D |
| ATOM | 7317 | C | LEU D | 469 | 140.005 | 8.574 | 37.960 | 1.00 | 24.60 | D |
| ATOM | 7318 | O | LEU D | 469 | 139.162 | 7.729 | 37.690 | 1.00 | 24.12 | D |
| ATOM | 7319 | N | ASP D | 470 | 139.818 | 9.853 | 37.693 | 1.00 | 24.30 | D |
| ATOM | 7320 | CA | ASP D | 470 | 138.584 | 10.242 | 37.043 | 1.00 | 24.73 | D |
| ATOM | 7321 | CB | ASP D | 470 | 137.540 | 10.594 | 38.118 | 1.00 | 24.80 | D |
| ATOM | 7322 | CG | ASP D | 470 | 136.109 | 10.685 | 37.571 | 1.00 | 24.84 | D |
| ATOM | 7323 | OD1 | ASP D | 470 | 135.842 | 10.240 | 36.441 | 1.00 | 25.89 | D |
| ATOM | 7324 | OD2 | ASP D | 470 | 135.242 | 11.197 | 38.296 | 1.00 | 23.60 | D |
| ATOM | 7325 | C | ASP D | 470 | 138.857 | 11.409 | 36.091 | 1.00 | 24.57 | D |
| ATOM | 7326 | O | ASP D | 470 | 139.863 | 12.118 | 36.214 | 1.00 | 23.14 | D |
| ATOM | 7327 | N | ILE D | 471 | 137.988 | 11.584 | 35.110 | 1.00 | 24.45 | D |
| ATOM | 7328 | CA | ILE D | 471 | 138.184 | 12.684 | 34.197 | 1.00 | 26.31 | D |
| ATOM | 7329 | CB | ILE D | 471 | 138.389 | 12.210 | 32.737 | 1.00 | 24.06 | D |
| ATOM | 7330 | CG2 | ILE D | 471 | 138.861 | 13.393 | 31.915 | 1.00 | 24.13 | D |
| ATOM | 7331 | CG1 | ILE D | 471 | 139.454 | 11.097 | 32.656 | 1.00 | 22.43 | D |
| ATOM | 7332 | CD1 | ILE D | 471 | 139.553 | 10.449 | 31.293 | 1.00 | 18.18 | D |
| ATOM | 7333 | C | ILE D | 471 | 136.971 | 13.600 | 34.271 | 1.00 | 28.56 | D |
| ATOM | 7334 | O | ILE D | 471 | 135.833 | 13.135 | 34.280 | 1.00 | 30.74 | D |
| ATOM | 7335 | N | VAL D | 472 | 137.222 | 14.901 | 34.358 | 1.00 | 30.47 | D |
| ATOM | 7336 | CA | VAL D | 472 | 136.157 | 15.892 | 34.433 | 1.00 | 31.86 | D |
| ATOM | 7337 | CB | VAL D | 472 | 136.105 | 16.544 | 35.857 | 1.00 | 31.83 | D |
| ATOM | 7338 | CG1 | VAL D | 472 | 135.793 | 15.474 | 36.896 | 1.00 | 30.28 | D |
| ATOM | 7339 | CG2 | VAL D | 472 | 137.427 | 17.231 | 36.193 | 1.00 | 27.41 | D |
| ATOM | 7340 | C | VAL D | 472 | 136.369 | 16.966 | 33.358 | 1.00 | 33.87 | D |
| ATOM | 7341 | O | VAL D | 472 | 137.474 | 17.119 | 32.826 | 1.00 | 34.57 | D |
| ATOM | 7342 | N | GLU D | 473 | 135.304 | 17.692 | 33.026 | 1.00 | 35.69 | D |
| ATOM | 7343 | CA | GLU D | 473 | 135.354 | 18.747 | 32.004 | 1.00 | 37.97 | D |
| ATOM | 7344 | CE | GLU D | 473 | 134.565 | 18.321 | 30.751 | 1.00 | 39.26 | D |
| ATOM | 7345 | CG | GLU D | 473 | 134.587 | 19.326 | 29.567 | 1.00 | 41.82 | D |
| ATOM | 7346 | CD | GLU D | 473 | 133.581 | 18.966 | 28.455 | 1.00 | 41.92 | D |
| ATOM | 7347 | OE1 | GLU D | 473 | 133.651 | 17.853 | 27.893 | 1.00 | 41.68 | D |
| ATOM | 7348 | OE2 | GLU D | 473 | 132.712 | 19.801 | 28.145 | 1.00 | 42.75 | D |
| ATOM | 7349 | C | GLU D | 473 | 134.722 | 19.983 | 32.620 | 1.00 | 38.63 | D |
| ATOM | 7350 | O | GLU D | 473 | 133.585 | 19.944 | 33.084 | 1.00 | 39.66 | D |
| ATOM | 7351 | N | TYR D | 474 | 135.464 | 21.077 | 32.651 | 1.00 | 39.16 | D |
| ATOM | 7352 | CA | TYR D | 474 | 134.952 | 22.311 | 33.231 | 1.00 | 39.98 | D |
| ATOM | 7353 | CB | TYR D | 474 | 135.534 | 22.496 | 34.630 | 1.00 | 39.54 | D |
| ATOM | 7354 | CG | TYR D | 474 | 135.126 | 23.777 | 35.311 | 1.00 | 40.62 | D |
| ATOM | 7355 | CD1 | TYR D | 474 | 133.802 | 24.002 | 35.686 | 1.00 | 40.93 | D |
| ATOM | 7356 | CE1 | TYR D | 474 | 133.432 | 25.174 | 36.332 | 1.00 | 40.40 | D |
| ATOM | 7357 | CD2 | TYR D | 474 | 136.072 | 24.761 | 35.601 | 1.00 | 40.52 | D |
| ATOM | 7358 | CE2 | TYR D | 474 | 135.714 | 25.933 | 36.243 | 1.00 | 40.34 | D |
| ATOM | 7359 | CZ | TYR D | 474 | 134.393 | 26.134 | 36.604 | 1.00 | 40.71 | D |
| ATOM | 7360 | OH | TYR D | 474 | 134.025 | 27.300 | 37.224 | 1.00 | 41.79 | D |
| ATOM | 7361 | C | TYR D | 474 | 135.422 | 23.394 | 32.277 | 1.00 | 39.99 | D |
| ATOM | 7362 | O | TYR D | 474 | 136.550 | 23.339 | 31.784 | 1.00 | 40.02 | D |
| ATOM | 7363 | N | LYS D | 475 | 134.551 | 24.357 | 31.998 | 1.00 | 40.09 | D |
| ATOM | 7364 | CA | LYE D | 475 | 134.868 | 25.438 | 31.069 | 1.00 | 42.55 | D |
| ATOM | 7365 | CE | LYE D | 475 | 135.796 | 26.471 | 31.723 | 1.00 | 43.10 | D |
| ATOM | 7366 | CG | LYS D | 475 | 135.185 | 27.292 | 32.864 | 1.00 | 43.55 | D |
| ATOM | 7367 | CD | LYS D | 475 | 136.276 | 28.148 | 33.502 | 1.00 | 46.85 | D |
| ATOM | 7368 | CE | LYE D | 475 | 135.765 | 29.052 | 34.623 | 1.00 | 48.93 | D |
| ATOM | 7369 | NZ | LYE D | 475 | 134.765 | 30.077 | 34.181 | 1.00 | 49.55 | D |
| ATOM | 7370 | C | LYE D | 475 | 135.529 | 24.888 | 29.804 | 1.00 | 43.23 | D |
| ATOM | 7371 | O | LYE D | 475 | 136.556 | 25.401 | 29.352 | 1.00 | 43.08 | D |
| ATOM | 7372 | N | ASN D | 476 | 134.943 | 23.831 | 29.248 | 1.00 | 44.27 | D |
| ATOM | 7373 | CA | ASN D | 476 | 135.449 | 23.202 | 28.024 | 1.00 | 44.54 | D |
| ATOM | 7374 | CB | ASN D | 476 | 135.383 | 24.180 | 26.850 | 1.00 | 44.94 | D |
| ATOM | 7375 | CG | ASN D | 476 | 134.054 | 24.135 | 26.140 | 1.00 | 44.85 | D |
| ATOM | 7376 | OD1 | ASN D | 476 | 133.040 | 24.596 | 26.665 | 1.00 | 44.09 | D |
| ATOM | 7377 | ND2 | ASN D | 476 | 134.048 | 23.565 | 24.934 | 1.00 | 45.89 | D |
| ATOM | 7378 | C | ASN D | 476 | 136.841 | 22.591 | 28.077 | 1.00 | 44.03 | D |
| ATOM | 7379 | O | ASN D | 476 | 137.403 | 22.230 | 27.036 | 1.00 | 44.45 | D |
| ATOM | 7380 | N | ILE D | 477 | 137.400 | 22.474 | 29.280 | 1.00 | 43.98 | D |
| ATOM | 7381 | CA | ILE D | 477 | 138.722 | 21.860 | 29.459 | 1.00 | 42.39 | D |
| ATOM | 7382 | CB | ILE D | 477 | 139.681 | 22.744 | 30.260 | 1.00 | 42.82 | D |
| ATOM | 7383 | CG2 | ILE D | 477 | 141.024 | 22.052 | 30.387 | 1.00 | 43.06 | D |
| ATOM | 7384 | CG1 | ILE D | 477 | 139.842 | 24.097 | 29.583 | 1.00 | 42.36 | D |
| ATOM | 7385 | CD1 | ILE D | 477 | 140.465 | 25.135 | 30.486 | 1.00 | 41.94 | D |
| ATOM | 7386 | C | ILE D | 477 | 138.528 | 20.578 | 30.249 | 1.00 | 41.19 | D |
| ATOM | 7387 | O | ILE D | 477 | 137.805 | 20.560 | 31.253 | 1.00 | 40.43 | D |
| ATOM | 7388 | N | LYS D | 478 | 139.166 | 19.511 | 29.777 | 1.00 | 38.84 | D |
| ATOM | 7389 | CA | LYS D | 478 | 139.078 | 18.212 | 30.428 | 1.00 | 36.96 | D |
| ATOM | 7390 | CB | LYS D | 478 | 139.070 | 17.085 | 29.394 | 1.00 | 35.75 | D |
| ATOM | 7391 | CG | LYS D | 478 | 137.740 | 16.932 | 28.687 | 1.00 | 35.23 | D |
| ATOM | 7392 | CD | LYS D | 478 | 137.557 | 15.519 | 28.160 | 1.00 | 32.30 | D |
| ATOM | 7393 | CE | LYS D | 478 | 136.120 | 15.312 | 27.725 | 1.00 | 31.88 | D |
| ATOM | 7394 | NZ | LYS D | 478 | 135.743 | 13.871 | 27.552 | 1.00 | 32.71 | D |
| ATOM | 7395 | C | LYS D | 478 | 140.218 | 17.984 | 31.402 | 1.00 | 36.46 | D |
| ATOM | 7396 | O | LYS D | 478 | 141.381 | 18.009 | 31.015 | 1.00 | 36.77 | D |
| ATOM | 7397 | N | TYR D | 479 | 139.884 | 17.749 | 32.666 | 1.00 | 35.14 | D |
| ATOM | 7398 | CA | TYR D | 479 | 140.906 | 17.514 | 33.675 | 1.00 | 34.37 | D |
| ATOM | 7399 | CB | TYR D | 479 | 140.674 | 18.442 | 34.875 | 1.00 | 33.93 | D |
| ATOM | 7400 | CG | TYR D | 479 | 140.847 | 19.914 | 34.543 | 1.00 | 34.98 | D |
| ATOM | 7401 | CD1 | TYR D | 479 | 142.108 | 20.529 | 34.614 | 1.00 | 33.05 | D |
| ATOM | 7402 | CE1 | TYR D | 479 | 142.264 | 21.880 | 34.300 | 1.00 | 32.76 | D |
| ATOM | 7403 | CD2 | TYR D | 479 | 139.753 | 20.691 | 34.147 | 1.00 | 33.92 | D |
| ATOM | 7404 | CE2 | TYR D | 479 | 139.901 | 22.037 | 33.830 | 1.00 | 34.11 | D |
| ATOM | 7405 | CZ | TYR D | 479 | 141.152 | 22.624 | 33.907 | 1.00 | 34.22 | D |
| ATOM | 7406 | OH | TYR D | 479 | 141.270 | 23.952 | 33.573 | 1.00 | 37.80 | D |
| ATOM | 7407 | C | TYR D | 479 | 140.936 | 16.061 | 34.134 | 1.00 | 32.92 | D |
| ATOM | 7408 | O | TYR D | 479 | 139.903 | 15.404 | 34.218 | 1.00 | 32.94 | D |
| ATOM | 7409 | N | ILE D | 480 | 142.138 | 15.569 | 34.412 | 1.00 | 31.23 | D |
| ATOM | 7410 | CA | ILE D | 480 | 142.344 | 14.213 | 34.910 | 1.00 | 30.00 | D |
| ATOM | 7411 | CB | ILE D | 480 | 143.568 | 13.528 | 34.236 | 1.00 | 30.27 | D |
| ATOM | 7412 | CG2 | ILE D | 480 | 143.826 | 12.150 | 34.879 | 1.00 | 31.07 | D |
| ATOM | 7413 | CG1 | ILE D | 480 | 143.335 | 13.334 | 32.741 | 1.00 | 31.12 | D |
| ATOM | 7414 | CD1 | ILE D | 480 | 144.440 | 12.531 | 32.060 | 1.00 | 26.66 | D |
| ATOM | 7415 | C | ILE D | 480 | 142.674 | 14.314 | 36.417 | 1.00 | 29.96 | D |
| ATOM | 7416 | O | ILE D | 480 | 143.583 | 15.063 | 36.808 | 1.00 | 29.34 | D |
| ATOM | 7417 | N | VAL D | 481 | 141.944 | 13.582 | 37.263 | 1.00 | 28.07 | D |
| ATOM | 7418 | CA | VAL D | 481 | 142.234 | 13.590 | 38.702 | 1.00 | 26.17 | D |
| ATOM | 7419 | CB | VAL D | 481 | 141.048 | 14.099 | 39.550 | 1.00 | 25.50 | D |
| ATOM | 7420 | CG1 | VAL D | 481 | 141.533 | 14.438 | 40.943 | 1.00 | 25.80 | D |
| ATOM | 7421 | CG2 | VAL D | 481 | 140.431 | 15.338 | 38.910 | 1.00 | 26.04 | D |
| ATOM | 7422 | C | VAL D | 481 | 142.587 | 12.166 | 39.110 | 1.00 | 26.07 | D |
| ATOM | 7423 | O | VAL D | 481 | 141.848 | 11.232 | 38.812 | 1.00 | 26.50 | D |
| ATOM | 7424 | N | THR D | 482 | 143.734 | 12.006 | 39.766 | 1.00 | 26.06 | D |
| ATOM | 7425 | CA | THR D | 482 | 144.217 | 10.691 | 40.184 | 1.00 | 26.60 | D |
| ATOM | 7426 | CB | THR D | 482 | 145.520 | 10.324 | 39.440 | 1.00 | 25.75 | D |
| ATOM | 7427 | OG1 | THR D | 482 | 146.452 | 11.402 | 39.568 | 1.00 | 23.78 | D |
| ATOM | 7428 | CG2 | THR D | 482 | 145.252 | 10.057 | 37.971 | 1.00 | 26.32 | D |
| ATOM | 7429 | C | THR D | 482 | 144.496 | 10.591 | 41.688 | 1.00 | 27.46 | D |
| ATOM | 7430 | O | THR D | 482 | 145.180 | 11.444 | 42.267 | 1.00 | 29.29 | D |
| ATOM | 7431 | N | GLY D | 483 | 143.977 | 9.530 | 42.303 | 1.00 | 25.97 | D |
| ATOM | 7432 | CA | GLY D | 483 | 144.176 | 9.303 | 43.724 | 1.00 | 24.32 | D |
| ATOM | 7433 | C | GLY D | 483 | 145.201 | 8.206 | 43.922 | 1.00 | 23.49 | D |
| ATOM | 7434 | O | GLY D | 483 | 145.156 | 7.171 | 43.236 | 1.00 | 23.98 | D |
| ATOM | 7435 | N | SER D | 484 | 146.106 | 8.428 | 44.877 | 1.00 | 22.04 | D |
| ATOM | 7436 | CA | SER D | 484 | 147.208 | 7.503 | 45.153 | 1.00 | 21.89 | D |
| ATOM | 7437 | CB | SER D | 484 | 148.540 | 8.193 | 44.820 | 1.00 | 19.14 | D |
| ATOM | 7438 | OG | SER D | 484 | 149.640 | 7.324 | 45.038 | 1.00 | 17.25 | D |
| ATOM | 7439 | C | SER D | 484 | 147.266 | 7.012 | 46.582 | 1.00 | 22.62 | D |
| ATOM | 7440 | O | SER D | 484 | 146.488 | 7.433 | 47.426 | 1.00 | 25.82 | D |
| ATOM | 7441 | N | ARG D | 485 | 148.193 | 6.100 | 46.837 | 1.00 | 24.32 | D |
| ATOM | 7442 | CA | ARG D | 485 | 148.412 | 5.585 | 48.179 | 1.00 | 26.12 | D |
| ATOM | 7443 | CB | ARG D | 485 | 149.052 | 4.206 | 48.134 | 1.00 | 27.36 | D |
| ATOM | 7444 | CG | ARG D | 485 | 148.168 | 3.160 | 47.556 | 1.00 | 28.76 | D |
| ATOM | 7445 | CD | ARG D | 485 | 148.786 | 1.809 | 47.722 | 1.00 | 29.11 | D |
| ATOM | 7446 | NE | ARG D | 485 | 147.768 | 0.784 | 47.543 | 1.00 | 31.30 | D |
| ATOM | 7447 | CZ | ARG D | 485 | 148.031 | −0.500 | 47.350 | 1.00 | 31.33 | D |
| ATOM | 7448 | NH1 | ARG D | 485 | 149.283 | −0.931 | 47.304 | 1.00 | 30.94 | D |
| ATOM | 7449 | NH2 | ARG D | 485 | 147.039 | −1.353 | 47.214 | 1.00 | 31.96 | D |
| ATOM | 7450 | C | ARG D | 485 | 149.367 | 6.534 | 48.886 | 1.00 | 27.46 | D |
| ATOM | 7451 | O | ARG D | 485 | 149.712 | 6.329 | 50.045 | 1.00 | 28.74 | D |
| ATOM | 7452 | N | ASP D | 486 | 149.822 | 7.561 | 48.186 | 1.00 | 27.88 | D |
| ATOM | 7453 | CA | ASP D | 486 | 150.722 | 8.487 | 48.819 | 1.00 | 27.38 | D |
| ATOM | 7454 | CB | ASP D | 486 | 151.631 | 9.137 | 47.789 | 1.00 | 29.39 | D |
| ATOM | 7455 | CG | ASP D | 486 | 150.858 | 9.822 | 46.695 | 1.00 | 30.30 | D |
| ATOM | 7456 | OD1 | ASP D | 486 | 149.693 | 10.190 | 46.951 | 1.00 | 28.83 | D |
| ATOM | 7457 | OD2 | ASP D | 486 | 151.410 | 10.006 | 45.591 | 1.00 | 33.15 | D |
| ATOM | 7458 | C | ASP D | 486 | 149.947 | 9.547 | 49.566 | 1.00 | 26.08 | D |
| ATOM | 7459 | O | ASP D | 486 | 150.524 | 10.533 | 49.997 | 1.00 | 26.51 | D |
| ATOM | 7460 | N | ASN D | 487 | 148.643 | 9.343 | 49.727 | 1.00 | 26.47 | D |
| ATOM | 7461 | CA | ASN D | 487 | 147.778 | 10.295 | 50.449 | 1.00 | 28.03 | D |
| ATOM | 7462 | CB | ASN D | 487 | 148.466 | 10.859 | 51.720 | 1.00 | 24.22 | D |
| ATOM | 7463 | CG | ASN D | 487 | 149.130 | 9.783 | 52.608 | 1.00 | 25.85 | D |
| ATOM | 7464 | OD1 | ASN D | 487 | 150.077 | 10.094 | 53.357 | 1.00 | 25.75 | D |
| ATOM | 7465 | ND2 | ASN D | 487 | 148.639 | 8.536 | 52.543 | 1.00 | 23.88 | D |
| ATOM | 7466 | C | ASN D | 487 | 147.331 | 11.492 | 49.578 | 1.00 | 29.17 | D |
| ATOM | 7467 | O | ASN D | 487 | 146.419 | 12.233 | 49.958 | 1.00 | 30.88 | D |
| ATOM | 7468 | N | THR D | 488 | 147.959 | 11.686 | 48.419 | 1.00 | 30.51 | D |
| ATOM | 7469 | CA | THR D | 488 | 147.599 | 12.817 | 47.574 | 1.00 | 31.76 | D |
| ATOM | 7470 | CB | THR D | 488 | 148.867 | 13.590 | 47.044 | 1.00 | 32.19 | D |
| ATOM | 7471 | OG1 | THR D | 488 | 149.361 | 12.984 | 45.841 | 1.00 | 30.87 | D |
| ATOM | 7472 | CG2 | THR D | 488 | 149.968 | 13.586 | 48.078 | 1.00 | 32.86 | D |
| ATOM | 7473 | C | THR D | 488 | 146.708 | 12.444 | 46.387 | 1.00 | 33.20 | D |
| ATOM | 7474 | O | THR D | 488 | 146.374 | 11.267 | 46.172 | 1.00 | 32.98 | D |
| ATOM | 7475 | N | LEU D | 489 | 146.310 | 13.480 | 45.650 | 1.00 | 34.03 | D |
| ATOM | 7476 | CA | LEU D | 489 | 145.468 | 13.377 | 44.465 | 1.00 | 34.19 | D |
| ATOM | 7477 | CS | LEU D | 489 | 144.056 | 13.869 | 44.779 | 1.00 | 34.15 | D |
| ATOM | 7478 | CG | LEU D | 489 | 143.138 | 12.961 | 45.589 | 1.00 | 34.12 | D |
| ATOM | 7479 | CD1 | LEU D | 489 | 142.459 | 13.747 | 46.710 | 1.00 | 33.50 | D |
| ATOM | 7480 | CD2 | LEU D | 489 | 142.102 | 12.369 | 44.642 | 1.00 | 32.16 | D |
| ATOM | 7481 | C | LEU D | 489 | 146.084 | 14.320 | 43.448 | 1.00 | 33.40 | D |
| ATOM | 7482 | O | LEU D | 489 | 145.854 | 15.519 | 43.533 | 1.00 | 34.39 | D |
| ATOM | 7483 | N | HIS D | 490 | 146.876 | 13.818 | 42.506 | 1.00 | 32.48 | D |
| ATOM | 7484 | CA | HIS D | 490 | 147.465 | 14.727 | 41.522 | 1.00 | 31.60 | D |
| ATOM | 7485 | CB | HIS D | 490 | 148.744 | 14.136 | 40.909 | 1.00 | 31.07 | D |
| ATOM | 7486 | CG | HIS D | 490 | 149.914 | 14.125 | 41.846 | 1.00 | 31.65 | D |
| ATOM | 7487 | CD2 | HIS D | 490 | 149.977 | 14.042 | 43.197 | 1.00 | 32.29 | D |
| ATOM | 7488 | ND1 | HIS D | 490 | 151.219 | 14.191 | 41.407 | 1.00 | 33.73 | D |
| ATOM | 7489 | CE1 | HIS D | 490 | 152.036 | 14.152 | 42.446 | 1.00 | 33.37 | D |
| ATOM | 7490 | NE2 | HIS D | 490 | 151.306 | 14.061 | 43.543 | 1.00 | 32.68 | D |
| ATOM | 7491 | C | HIS D | 490 | 146.456 | 15.034 | 40.429 | 1.00 | 30.39 | D |
| ATOM | 7492 | O | HIS D | 490 | 145.735 | 14.149 | 39.983 | 1.00 | 31.27 | D |
| ATOM | 7493 | N | VAL D | 491 | 146.391 | 16.296 | 40.022 | 1.00 | 29.80 | D |
| ATOM | 7494 | CA | VAL D | 491 | 145.466 | 16.726 | 38.977 | 1.00 | 31.12 | D |
| ATOM | 7495 | CB | VAL D | 491 | 144.610 | 17.941 | 39.472 | 1.00 | 30.67 | D |
| ATOM | 7496 | CG1 | VAL D | 491 | 143.683 | 18.442 | 38.365 | 1.00 | 29.26 | D |
| ATOM | 7497 | CG2 | VAL D | 491 | 143.798 | 17.544 | 40.683 | 1.00 | 29.19 | D |
| ATOM | 7498 | C | VAL D | 491 | 146.256 | 17.127 | 37.712 | 1.00 | 32.27 | D |
| ATOM | 7499 | O | VAL D | 491 | 147.249 | 17.850 | 37.802 | 1.00 | 33.21 | D |
| ATOM | 7500 | N | TRP D | 492 | 145.829 | 16.647 | 36.545 | 1.00 | 32.46 | D |
| ATOM | 7501 | CA | TRP D | 492 | 146.501 | 16.975 | 35.285 | 1.00 | 33.55 | D |
| ATOM | 7502 | CB | TRP D | 492 | 147.308 | 15.798 | 34.759 | 1.00 | 33.18 | D |
| ATOM | 7503 | CG | TRP D | 492 | 147.697 | 14.828 | 35.788 | 1.00 | 34.51 | D |
| ATOM | 7504 | CD2 | TRP D | 492 | 149.014 | 14.604 | 36.288 | 1.00 | 35.06 | D |
| ATOM | 7505 | CE2 | TRP D | 492 | 148.930 | 13.552 | 37.224 | 1.00 | 34.88 | D |
| ATOM | 7506 | CE3 | TRP D | 492 | 150.263 | 15.189 | 36.034 | 1.00 | 33.09 | D |
| ATOM | 7507 | CD1 | TRP D | 492 | 146.884 | 13.936 | 36.423 | 1.00 | 34.18 | D |
| ATOM | 7508 | NE1 | TRP D | 492 | 147.617 | 13.161 | 37.285 | 1.00 | 34.58 | D |
| ATOM | 7509 | CZ2 | TRP D | 492 | 150.048 | 13.073 | 37.906 | 1.00 | 34.36 | D |
| ATOM | 7510 | CZ3 | TRP D | 492 | 151.371 | 14.715 | 36.710 | 1.00 | 32.50 | D |
| ATOM | 7511 | CH2 | TRP D | 492 | 151.258 | 13.667 | 37.634 | 1.00 | 33.38 | D |
| ATOM | 7512 | C | TRP D | 492 | 145.475 | 17.318 | 34.226 | 1.00 | 34.32 | D |
| ATOM | 7513 | O | TRP D | 492 | 144.338 | 16.864 | 34.292 | 1.00 | 35.53 | D |
| ATOM | 7514 | N | LYS D | 493 | 145.859 | 18.110 | 33.236 | 1.00 | 34.28 | D |
| ATOM | 7515 | CA | LYS D | 493 | 144.900 | 18.433 | 32.202 | 1.00 | 35.06 | D |
| ATOM | 7516 | CB | LYS D | 493 | 145.116 | 19.860 | 31.694 | 1.00 | 35.38 | D |
| ATOM | 7517 | CG | LYS D | 493 | 146.531 | 20.133 | 31.281 | 1.00 | 37.78 | D |
| ATOM | 7518 | CD | LYS D | 493 | 146.739 | 21.563 | 30.810 | 1.00 | 39.13 | D |
| ATOM | 7519 | CE | LYS D | 493 | 146.643 | 22.588 | 31.928 | 1.00 | 37.56 | D |
| ATOM | 7520 | NZ | LYS D | 493 | 147.094 | 23.906 | 31.410 | 1.00 | 35.80 | D |
| ATOM | 7521 | C | LYS D | 493 | 145.075 | 17.408 | 31.084 | 1.00 | 34.97 | D |
| ATOM | 7522 | O | LYS D | 493 | 146.182 | 17.192 | 30.600 | 1.00 | 33.77 | D |
| ATOM | 7523 | N | LEU D | 494 | 143.981 | 16.753 | 30.706 | 1.00 | 36.70 | D |
| ATOM | 7524 | CA | LEU D | 494 | 144.018 | 15.744 | 29.659 | 1.00 | 39.45 | D |
| ATOM | 7525 | CB | LEU D | 494 | 142.630 | 15.126 | 29.458 | 1.00 | 39.18 | D |
| ATOM | 7526 | CG | LEU D | 494 | 142.458 | 14.046 | 28.380 | 1.00 | 38.09 | D |
| ATOM | 7527 | CD1 | LEU D | 494 | 143.506 | 12.972 | 28.552 | 1.00 | 36.77 | D |
| ATOM | 7528 | CD2 | LEU D | 494 | 141.043 | 13.442 | 28.448 | 1.00 | 36.96 | D |
| ATOM | 7529 | C | LEU D | 494 | 144.493 | 16.382 | 28.366 | 1.00 | 42.52 | D |
| ATOM | 7530 | O | LEU D | 494 | 143.928 | 17.387 | 27.912 | 1.00 | 42.98 | D |
| ATOM | 7531 | N | PRO D | 495 | 145.559 | 15.819 | 27.764 | 1.00 | 45.09 | D |
| ATOM | 7532 | CD | PRO D | 495 | 146.315 | 14.686 | 28.330 | 1.00 | 45.01 | D |
| ATOM | 7533 | CA | PRO D | 495 | 146.168 | 16.287 | 26.505 | 1.00 | 45.59 | D |
| ATOM | 7534 | CB | PRO D | 495 | 147.255 | 15.253 | 26.241 | 1.00 | 45.10 | D |
| ATOM | 7535 | CG | PRO D | 495 | 147.633 | 14.802 | 27.619 | 1.00 | 45.87 | D |
| ATOM | 7536 | C | PRO D | 495 | 145.146 | 16.324 | 25.365 | 1.00 | 46.50 | D |
| ATOM | 7537 | O | PRO D | 495 | 144.334 | 15.409 | 25.243 | 1.00 | 47.18 | D |
| ATOM | 7538 | N | LYS D | 496 | 145.194 | 17.360 | 24.525 | 1.00 | 47.14 | D |
| ATOM | 7539 | CA | LYS D | 496 | 144.254 | 17.491 | 23.400 | 1.00 | 47.35 | D |
| ATOM | 7540 | CB | LYS D | 496 | 143.768 | 18.942 | 23.268 | 1.00 | 47.30 | D |
| ATOM | 7541 | CG | LYS D | 496 | 143.109 | 19.501 | 24.520 | 0.00 | 47.88 | D |
| ATOM | 7542 | CD | LYS D | 496 | 141.811 | 18.778 | 24.840 | 0.00 | 48.23 | D |
| ATOM | 7543 | CE | LYS D | 496 | 141.133 | 19.370 | 26.065 | 0.00 | 48.50 | D |
| ATOM | 7544 | NZ | LYS D | 496 | 139.817 | 18.722 | 26.331 | 0.00 | 48.74 | D |
| ATOM | 7545 | C | LYS D | 496 | 144.853 | 17.046 | 22.064 | 1.00 | 47.41 | D |
| ATOM | 7546 | O | LYS D | 496 | 145.277 | 15.898 | 21.895 | 1.00 | 46.92 | D |
| ATOM | 7547 | N | ASP D | 508 | 157.546 | 8.144 | 18.136 | 1.00 | 82.08 | D |
| ATOM | 7548 | CA | ASP D | 508 | 158.190 | 8.937 | 19.182 | 1.00 | 81.66 | D |
| ATOM | 7549 | CB | ASP D | 508 | 158.981 | 10.090 | 18.569 | 1.00 | 82.86 | D |
| ATOM | 7550 | CG | ASP D | 508 | 158.086 | 11.249 | 18.168 | 1.00 | 83.90 | D |
| ATOM | 7551 | OD1 | ASP D | 508 | 157.135 | 11.025 | 17.381 | 1.00 | 83.92 | D |
| ATOM | 7552 | OD2 | ASP D | 508 | 158.334 | 12.378 | 18.644 | 1.00 | 83.63 | D |
| ATOM | 7553 | C | ASP D | 508 | 157.121 | 9.528 | 20.096 | 1.00 | 80.44 | D |
| ATOM | 7554 | O | ASP D | 508 | 157.419 | 10.346 | 20.972 | 1.00 | 80.05 | D |
| ATOM | 7555 | N | TYR D | 509 | 155.874 | 9.120 | 19.884 | 1.00 | 78.72 | D |
| ATOM | 7556 | CA | TYR D | 509 | 154.768 | 9.631 | 20.678 | 1.00 | 76.03 | D |
| ATOM | 7557 | CB | TYR D | 509 | 153.454 | 8.997 | 20.254 | 1.00 | 76.05 | D |
| ATOM | 7558 | CG | TYR D | 509 | 152.443 | 10.025 | 19.841 | 1.00 | 77.13 | D |
| ATOM | 7559 | CD1 | TYR D | 509 | 152.268 | 11.195 | 20.579 | 1.00 | 77.84 | D |
| ATOM | 7560 | CE1 | TYR D | 509 | 151.324 | 12.147 | 20.205 | 1.00 | 79.73 | D |
| ATOM | 7561 | CD2 | TYR D | 509 | 151.653 | 9.830 | 18.719 | 1.00 | 78.10 | D |
| ATOM | 7562 | CE2 | TYR D | 509 | 150.709 | 10.769 | 18.335 | 1.00 | 79.97 | D |
| ATOM | 7563 | CZ | TYR D | 509 | 150.543 | 11.923 | 19.080 | 1.00 | 80.67 | D |
| ATOM | 7564 | OH | TYR D | 509 | 149.570 | 12.832 | 18.715 | 1.00 | 82.17 | D |
| ATOM | 7565 | C | TYR D | 509 | 154.938 | 9.440 | 22.170 | 1.00 | 73.80 | D |
| ATOM | 7566 | O | TYR D | 509 | 154.457 | 10.253 | 22.960 | 1.00 | 75.57 | D |
| ATOM | 7567 | N | PRO D | 510 | 155.602 | 8.352 | 22.588 | 1.00 | 70.29 | D |
| ATOM | 7568 | CD | PRO D | 510 | 156.084 | 7.160 | 21.860 | 1.00 | 68.38 | D |
| ATOM | 7569 | CA | PRO D | 510 | 155.758 | 8.194 | 24.039 | 1.00 | 66.01 | D |
| ATOM | 7570 | CB | PRO D | 510 | 156.600 | 6.929 | 24.150 | 1.00 | 66.48 | D |
| ATOM | 7571 | CG | PRO D | 510 | 156.132 | 6.128 | 22.949 | 1.00 | 68.27 | D |
| ATOM | 7572 | C | PRO D | 510 | 156.446 | 9.424 | 24.644 | 1.00 | 62.34 | D |
| ATOM | 7573 | O | PRO D | 510 | 157.667 | 9.504 | 24.676 | 1.00 | 62.17 | D |
| ATOM | 7574 | N | LEU D | 511 | 155.655 | 10.392 | 25.096 | 1.00 | 58.50 | D |
| ATOM | 7575 | CA | LEU D | 511 | 156.197 | 11.603 | 25.700 | 1.00 | 54.92 | D |
| ATOM | 7576 | CB | LEU D | 511 | 155.354 | 12.806 | 25.313 | 1.00 | 54.10 | D |
| ATOM | 7577 | CG | LEU D | 511 | 155.130 | 12.940 | 23.807 | 1.00 | 52.83 | D |
| ATOM | 7578 | CD1 | LEU D | 511 | 154.228 | 14.114 | 23.521 | 1.00 | 51.33 | D |
| ATOM | 7579 | CD2 | LEU D | 511 | 156.467 | 13.102 | 23.107 | 1.00 | 52.54 | D |
| ATOM | 7580 | C | LEU D | 511 | 156.188 | 11.451 | 27.211 | 1.00 | 53.49 | D |
| ATOM | 7581 | O | LEU D | 511 | 155.145 | 11.576 | 27.847 | 1.00 | 53.00 | D |
| ATOM | 7582 | N | VAL D | 512 | 157.359 | 11.184 | 27.778 | 1.00 | 52.14 | D |
| ATOM | 7583 | CA | VAL D | 512 | 157.506 | 10.992 | 29.213 | 1.00 | 49.88 | D |
| ATOM | 7584 | CB | VAL D | 512 | 158.576 | 9.937 | 29.498 | 1.00 | 48.71 | D |
| ATOM | 7585 | CG1 | VAL D | 512 | 158.654 | 9.664 | 30.992 | 1.00 | 49.81 | D |
| ATOM | 7586 | CG2 | VAL D | 512 | 158.262 | 8.666 | 28.725 | 1.00 | 47.64 | D |
| ATOM | 7587 | C | VAL D | 512 | 157.889 | 12.265 | 29.954 | 1.00 | 49.28 | D |
| ATOM | 7588 | O | VAL D | 512 | 158.855 | 12.925 | 29.592 | 1.00 | 48.19 | D |
| ATOM | 7589 | N | PHE D | 513 | 157.126 | 12.598 | 30.994 | 1.00 | 50.24 | D |
| ATOM | 7590 | CA | PHE D | 513 | 157.397 | 13.778 | 31.823 | 1.00 | 51.82 | D |
| ATOM | 7591 | CB | PHE D | 513 | 156.158 | 14.671 | 31.950 | 1.00 | 51.83 | D |
| ATOM | 7592 | CG | PHE D | 513 | 155.565 | 15.091 | 30.632 | 1.00 | 52.04 | D |
| ATOM | 7593 | CD1 | PHE D | 513 | 154.849 | 14.187 | 29.856 | 1.00 | 52.52 | D |
| ATOM | 7594 | CD2 | PHE D | 513 | 155.724 | 16.392 | 30.164 | 1.00 | 51.87 | D |
| ATOM | 7595 | CE1 | PHE D | 513 | 154.305 | 14.571 | 28.635 | 1.00 | 52.41 | D |
| ATOM | 7596 | CE2 | PHE D | 513 | 155.182 | 16.785 | 28.944 | 1.00 | 51.44 | D |
| ATOM | 7597 | CZ | PHE D | 513 | 154.472 | 15.873 | 28.181 | 1.00 | 51.72 | D |
| ATOM | 7598 | C | PHE D | 513 | 157.807 | 13.318 | 33.225 | 1.00 | 52.58 | D |
| ATOM | 7599 | O | PHE D | 513 | 156.957 | 13.164 | 34.100 | 1.00 | 53.73 | D |
| ATOM | 7600 | N | HIS D | 514 | 159.107 | 13.108 | 33.427 | 1.00 | 52.88 | D |
| ATOM | 7601 | CA | HIS D | 514 | 159.649 | 12.656 | 34.707 | 1.00 | 52.63 | D |
| ATOM | 7602 | CB | HIS D | 514 | 161.150 | 12.425 | 34.577 | 1.00 | 52.45 | D |
| ATOM | 7603 | CG | HIS D | 514 | 161.495 | 11.297 | 33.662 | 1.00 | 53.43 | D |
| ATOM | 7604 | CD2 | HIS D | 514 | 161.857 | 11.285 | 32.358 | 1.00 | 53.76 | D |
| ATOM | 7605 | ND1 | HIS D | 514 | 161.411 | 9.977 | 34.049 | 1.00 | 54.13 | D |
| ATOM | 7606 | CE1 | HIS D | 514 | 161.705 | 9.199 | 33.023 | 1.00 | 54.22 | D |
| ATOM | 7607 | NE2 | HIS D | 514 | 161.979 | 9.968 | 31.985 | 1.00 | 55.05 | D |
| ATOM | 7608 | C | HIS D | 514 | 159.388 | 13.615 | 35.855 | 1.00 | 53.18 | D |
| ATOM | 7609 | O | HIS D | 514 | 159.096 | 13.204 | 36.986 | 1.00 | 54.06 | D |
| ATOM | 7610 | N | THR D | 515 | 159.494 | 14.901 | 35.569 | 1.00 | 52.64 | D |
| ATOM | 7611 | CA | THR D | 515 | 159.281 | 15.888 | 36.597 | 1.00 | 52.40 | D |
| ATOM | 7612 | CB | THR D | 515 | 160.524 | 16.788 | 36.742 | 1.00 | 52.48 | D |
| ATOM | 7613 | OG1 | THR D | 515 | 160.166 | 17.996 | 37.420 | 1.00 | 52.59 | D |
| ATOM | 7614 | CG2 | THR D | 515 | 161.126 | 17.093 | 35.381 | 1.00 | 52.87 | D |
| ATOM | 7615 | C | THR D | 515 | 158.029 | 16.705 | 36.322 | 1.00 | 52.17 | D |
| ATOM | 7616 | O | THR D | 515 | 157.858 | 17.269 | 35.245 | 1.00 | 51.18 | D |
| ATOM | 7617 | N | PRO D | 516 | 157.114 | 16.736 | 37.304 | 1.00 | 53.39 | D |
| ATOM | 7618 | CD | PRO D | 516 | 157.164 | 15.841 | 38.478 | 1.00 | 53.89 | D |
| ATOM | 7619 | CA | PRO D | 516 | 155.835 | 17.449 | 37.271 | 1.00 | 52.84 | D |
| ATOM | 7620 | CB | PRO D | 516 | 155.231 | 17.119 | 38.632 | 1.00 | 52.59 | D |
| ATOM | 7621 | CG | PRO D | 516 | 155.703 | 15.729 | 38.851 | 1.00 | 52.83 | D |
| ATOM | 7622 | C | PRO D | 516 | 155.976 | 18.939 | 37.057 | 1.00 | 51.67 | D |
| ATOM | 7623 | O | PRO D | 516 | 155.238 | 19.533 | 36.279 | 1.00 | 52.06 | D |
| ATOM | 7624 | N | GLU D | 517 | 156.925 | 19.545 | 37.751 | 1.00 | 51.53 | D |
| ATOM | 7625 | CA | GLD D | 517 | 157.115 | 20.974 | 37.615 | 1.00 | 51.30 | D |
| ATOM | 7626 | CB | GLU D | 517 | 158.171 | 21.456 | 38.611 | 1.00 | 53.01 | D |
| ATOM | 7627 | CG | GLU D | 517 | 157.783 | 21.225 | 40.076 | 1.00 | 55.65 | D |
| ATOM | 7628 | CD | GLU D | 517 | 156.339 | 21.648 | 40.384 | 1.00 | 57.61 | D |
| ATOM | 7629 | OE1 | GLU D | 517 | 155.899 | 22.718 | 39.901 | 1.00 | 58.27 | D |
| ATOM | 7630 | OE2 | GLU D | 517 | 155.641 | 20.911 | 41.115 | 1.00 | 58.41 | D |
| ATOM | 7631 | C | GLU D | 517 | 157.482 | 21.376 | 36.180 | 1.00 | 50.73 | D |
| ATOM | 7632 | O | GLD D | 517 | 157.417 | 22.549 | 35.824 | 1.00 | 50.38 | D |
| ATOM | 7633 | N | GLU D | 518 | 157.853 | 20.408 | 35.350 | 1.00 | 49.94 | D |
| ATOM | 7634 | CA | GLU D | 518 | 158.200 | 20.707 | 33.964 | 1.00 | 48.95 | D |
| ATOM | 7635 | CB | GLU D | 518 | 159.552 | 20.081 | 33.613 | 1.00 | 49.87 | D |
| ATOM | 7636 | CG | GLU D | 518 | 160.741 | 20.801 | 34.239 | 1.00 | 51.31 | D |
| ATOM | 7637 | CD | GLU D | 518 | 162.074 | 20.125 | 33.945 | 1.00 | 52.52 | D |
| ATOM | 7638 | OE1 | GLU D | 518 | 162.225 | 19.579 | 32.823 | 1.00 | 53.98 | D |
| ATOM | 7639 | OE2 | GLU D | 518 | 162.971 | 20.153 | 34.826 | 1.00 | 50.01 | D |
| ATOM | 7640 | C | GLU D | 518 | 157.119 | 20.209 | 33.003 | 1.00 | 47.92 | D |
| ATOM | 7641 | O | GLU D | 518 | 157.143 | 20.496 | 31.808 | 1.00 | 47.01 | D |
| ATOM | 7642 | N | ASN D | 519 | 156.164 | 19.465 | 33.541 | 1.00 | 47.59 | D |
| ATOM | 7643 | CA | ASN D | 519 | 155.071 | 18.933 | 32.746 | 1.00 | 46.99 | D |
| ATOM | 7644 | CB | ASN D | 519 | 154.490 | 17.710 | 33.459 | 1.00 | 45.93 | D |
| ATOM | 7645 | CG | ASN D | 519 | 153.454 | 16.995 | 32.633 | 1.00 | 45.28 | D |
| ATOM | 7646 | OD1 | ASN D | 519 | 152.907 | 15.970 | 33.055 | 1.00 | 45.38 | D |
| ATOM | 7647 | ND2 | ASN D | 519 | 153.173 | 17.526 | 31.445 | 1.00 | 42.56 | D |
| ATOM | 7648 | C | ASN D | 519 | 153.998 | 20.023 | 32.551 | 1.00 | 46.69 | D |
| ATOM | 7649 | O | ASN D | 519 | 153.408 | 20.516 | 33.524 | 1.00 | 46.92 | D |
| ATOM | 7650 | N | PRO D | 520 | 153.758 | 20.431 | 31.291 | 1.00 | 45.28 | D |
| ATOM | 7651 | CD | PRO D | 520 | 154.544 | 20.037 | 30.110 | 1.00 | 43.44 | D |
| ATOM | 7652 | CA | PRO D | 520 | 152.769 | 21.460 | 30.937 | 1.00 | 44.93 | D |
| ATOM | 7653 | CB | PRO D | 520 | 152.965 | 21.621 | 29.431 | 1.00 | 43.10 | D |
| ATOM | 7654 | CG | PRO D | 520 | 154.396 | 21.232 | 29.234 | 1.00 | 43.83 | D |
| ATOM | 7655 | C | PRO D | 520 | 151.328 | 21.058 | 31.265 | 1.00 | 44.42 | D |
| ATOM | 7656 | O | PRO D | 520 | 150.430 | 21.899 | 31.341 | 1.00 | 45.05 | D |
| ATOM | 7657 | N | TYR D | 521 | 151.126 | 19.764 | 31.465 | 1.00 | 43.22 | D |
| ATOM | 7658 | CA | TYR D | 521 | 149.808 | 19.216 | 31.733 | 1.00 | 42.86 | D |
| ATOM | 7659 | CB | TYR D | 521 | 149.707 | 17.885 | 31.017 | 1.00 | 42.46 | D |
| ATOM | 7660 | CG | TYR D | 521 | 150.006 | 17.994 | 29.543 | 1.00 | 41.84 | D |
| ATOM | 7661 | CD1 | TYR D | 521 | 149.117 | 18.639 | 28.684 | 1.00 | 43.00 | D |
| ATOM | 7662 | CE1 | TYR D | 521 | 149.330 | 18.673 | 27.315 | 1.00 | 42.26 | D |
| ATOM | 7663 | CD2 | TYR D | 521 | 151.135 | 17.395 | 28.996 | 1.00 | 42.03 | D |
| ATOM | 7664 | CE2 | TYR D | 521 | 151.363 | 17.422 | 27.625 | 1.00 | 42.28 | D |
| ATOM | 7665 | CZ | TYR D | 521 | 150.445 | 18.058 | 26.788 | 1.00 | 43.30 | D |
| ATOM | 7666 | OH | TYR D | 521 | 150.592 | 18.015 | 25.418 | 1.00 | 44.00 | D |
| ATOM | 7667 | C | TYR D | 521 | 149.444 | 19.047 | 33.211 | 1.00 | 42.72 | D |
| ATOM | 7668 | O | TYR D | 521 | 148.267 | 18.922 | 33.567 | 1.00 | 42.06 | D |
| ATOM | 7669 | N | PHE D | 522 | 150.459 | 19.030 | 34.065 | 1.00 | 41.61 | D |
| ATOM | 7670 | CA | PHE D | 522 | 150.263 | 18.902 | 35.500 | 1.00 | 40.23 | D |
| ATOM | 7671 | CB | PHE D | 522 | 151.606 | 18.630 | 36.163 | 1.00 | 40.93 | D |
| ATOM | 7672 | CG | PHE D | 522 | 151.580 | 18.761 | 37.646 | 1.00 | 41.77 | D |
| ATOM | 7673 | CD1 | PHE D | 522 | 150.784 | 17.923 | 38.415 | 1.00 | 42.70 | D |
| ATOM | 7674 | CD2 | PHE D | 522 | 152.362 | 19.716 | 38.280 | 1.00 | 42.48 | D |
| ATOM | 7675 | CE1 | PHE D | 522 | 150.770 | 18.030 | 39.800 | 1.00 | 43.11 | D |
| ATOM | 7676 | CE2 | PHE D | 522 | 152.357 | 19.835 | 39.665 | 1.00 | 43.56 | D |
| ATOM | 7677 | CZ | PHE D | 522 | 151.559 | 18.989 | 40.427 | 1.00 | 44.10 | D |
| ATOM | 7678 | C | PHE D | 522 | 149.682 | 20.218 | 36.013 | 1.00 | 38.95 | D |
| ATOM | 7679 | O | PHE D | 522 | 149.994 | 21.271 | 35.470 | 1.00 | 38.64 | D |
| ATOM | 7680 | N | VAL D | 523 | 148.823 | 20.169 | 37.028 | 1.00 | 38.34 | D |
| ATOM | 7681 | CA | VAL D | 523 | 148.256 | 21.407 | 37.559 | 1.00 | 38.44 | D |
| ATOM | 7682 | CB | VAL D | 523 | 146.720 | 21.500 | 37.351 | 1.00 | 37.33 | D |
| ATOM | 7683 | CG1 | VAL D | 523 | 146.352 | 21.189 | 35.900 | 1.00 | 35.81 | D |
| ATOM | 7684 | CG2 | VAL D | 523 | 146.020 | 20.585 | 38.298 | 1.00 | 39.53 | D |
| ATOM | 7685 | C | VAL D | 523 | 148.543 | 21.590 | 39.044 | 1.00 | 38.09 | D |
| ATOM | 7686 | O | VAL D | 523 | 148.774 | 22.712 | 39.510 | 1.00 | 38.38 | D |
| ATOM | 7687 | N | GLY D | 524 | 148.543 | 20.484 | 39.780 | 1.00 | 36.48 | D |
| ATOM | 7688 | CA | GLY D | 524 | 148.798 | 20.567 | 41.203 | 1.00 | 35.67 | D |
| ATOM | 7689 | C | GLY D | 524 | 148.389 | 19.349 | 42.012 | 1.00 | 34.74 | D |
| ATOM | 7690 | O | GLY D | 524 | 147.705 | 18.446 | 41.512 | 1.00 | 36.14 | D |
| ATOM | 7691 | N | VAL D | 525 | 148.812 | 19.351 | 43.276 | 1.00 | 32.26 | D |
| ATOM | 7692 | CA | VAL D | 525 | 148.563 | 18.270 | 44.224 | 1.00 | 28.84 | D |
| ATOM | 7693 | CB | VAL D | 525 | 149.834 | 17.940 | 44.980 | 1.00 | 28.68 | D |
| ATOM | 7694 | CG1 | VAL D | 525 | 149.536 | 16.914 | 46.060 | 1.00 | 28.23 | D |
| ATOM | 7695 | CG2 | VAL D | 525 | 150.913 | 17.467 | 43.993 | 1.00 | 27.71 | D |
| ATOM | 7696 | C | VAL D | 525 | 147.522 | 18.613 | 45.261 | 1.00 | 27.86 | D |
| ATOM | 7697 | O | VAL D | 525 | 147.686 | 19.580 | 45.985 | 1.00 | 29.26 | D |
| ATOM | 7698 | N | LEU D | 526 | 146.469 | 17.807 | 45.349 | 1.00 | 26.79 | D |
| ATOM | 7699 | CA | LEU D | 526 | 145.421 | 18.028 | 46.341 | 1.00 | 24.97 | D |
| ATOM | 7700 | CB | LEU D | 526 | 144.082 | 17.539 | 45.796 | 1.00 | 24.09 | D |
| ATOM | 7701 | CG | LEU D | 526 | 143.105 | 18.496 | 45.072 | 1.00 | 25.53 | D |
| ATOM | 7702 | CD1 | LEU D | 526 | 143.706 | 19.861 | 44.714 | 1.00 | 21.96 | D |
| ATOM | 7703 | CD2 | LEU D | 526 | 142.600 | 17.770 | 43.842 | 1.00 | 24.82 | D |
| ATOM | 7704 | C | LEU D | 526 | 145.771 | 17.305 | 47.659 | 1.00 | 25.98 | D |
| ATOM | 7705 | O | LEU D | 526 | 145.389 | 16.153 | 47.865 | 1.00 | 26.01 | D |
| ATOM | 7706 | N | ARG D | 527 | 146.507 | 18.008 | 48.533 | 1.00 | 26.45 | D |
| ATOM | 7707 | CA | ARG D | 527 | 146.953 | 17.504 | 49.839 | 1.00 | 27.15 | D |
| ATOM | 7708 | CB | ARG D | 527 | 148.068 | 18.359 | 50.423 | 1.00 | 29.53 | D |
| ATOM | 7709 | CG | ARG D | 527 | 149.136 | 18.793 | 49.481 | 1.00 | 30.78 | D |
| ATOM | 7710 | CD | ARG D | 527 | 150.199 | 17.750 | 49.353 | 1.00 | 29.79 | D |
| ATOM | 7711 | NE | ARG D | 527 | 151.289 | 18.254 | 48.534 | 1.00 | 28.82 | D |
| ATOM | 7712 | CZ | ARG D | 527 | 152.426 | 17.609 | 48.341 | 1.00 | 28.06 | D |
| ATOM | 7713 | NH1 | ARG D | 527 | 152.621 | 16.424 | 48.913 | 1.00 | 27.74 | D |
| ATOM | 7714 | NH2 | ARG D | 527 | 153.361 | 18.156 | 47.578 | 1.00 | 28.96 | D |
| ATOM | 7715 | C | ARG D | 527 | 145.833 | 17.583 | 50.854 | 1.00 | 27.48 | D |
| ATOM | 7716 | O | ARG D | 527 | 145.198 | 18.625 | 50.982 | 1.00 | 28.10 | D |
| ATOM | 7717 | N | GLY D | 528 | 145.643 | 16.511 | 51.618 | 1.00 | 27.13 | D |
| ATOM | 7718 | CA | GLY D | 528 | 144.593 | 16.502 | 52.618 | 1.00 | 25.97 | D |
| ATOM | 7719 | C | GLY D | 528 | 144.378 | 15.186 | 53.350 | 1.00 | 27.12 | D |
| ATOM | 7720 | O | GLY D | 528 | 144.222 | 15.170 | 54.573 | 1.00 | 26.85 | D |
| ATOM | 7721 | N | HIS D | 529 | 144.349 | 14.076 | 52.619 | 1.00 | 28.35 | D |
| ATOM | 7722 | CA | HIS D | 529 | 144.148 | 12.771 | 53.242 | 1.00 | 28.78 | D |
| ATOM | 7723 | CB | HIS D | 529 | 143.686 | 11.747 | 52.197 | 1.00 | 29.36 | D |
| ATOM | 7724 | CG | HIS D | 529 | 142.272 | 11.942 | 51.729 | 1.00 | 31.01 | D |
| ATOM | 7725 | CD2 | HIS D | 529 | 141.776 | 12.234 | 50.502 | 1.00 | 32.50 | D |
| ATOM | 7726 | ND1 | HIS D | 529 | 141.181 | 11.808 | 52.562 | 1.00 | 29.94 | D |
| ATOM | 7727 | CE1 | HIS D | 529 | 140.074 | 12.010 | 51.864 | 1.00 | 32.77 | D |
| ATOM | 7728 | NE2 | HIS D | 529 | 140.409 | 12.270 | 50.613 | 1.00 | 31.73 | D |
| ATOM | 7729 | C | HIS D | 529 | 145.428 | 12.293 | 53.922 | 1.00 | 29.06 | D |
| ATOM | 7730 | O | HIS D | 529 | 146.525 | 12.716 | 53.562 | 1.00 | 29.97 | D |
| ATOM | 7731 | N | MSE D | 530 | 145.278 | 11.412 | 54.906 | 1.00 | 30.41 | D |
| ATOM | 7732 | CA | MSE D | 530 | 146.411 | 10.872 | 55.662 | 1.00 | 31.44 | D |
| ATOM | 7733 | CB | MSE D | 530 | 146.116 | 10.979 | 57.163 | 1.00 | 36.54 | D |
| ATOM | 7734 | CG | MSE D | 530 | 145.727 | 12.383 | 57.647 | 1.00 | 40.77 | D |
| ATOM | 7735 | SE | MSE D | 530 | 147.225 | 13.555 | 57.992 | 1.00 | 48.72 | D |
| ATOM | 7736 | CE | MSE D | 530 | 147.747 | 12.886 | 59.726 | 1.00 | 44.45 | D |
| ATOM | 7737 | C | MSE D | 530 | 146.708 | 9.409 | 55.286 | 1.00 | 30.15 | D |
| ATOM | 7738 | O | MSE D | 530 | 147.663 | 8.804 | 55.770 | 1.00 | 31.06 | D |
| ATOM | 7739 | N | ALA D | 531 | 145.872 | 8.837 | 54.433 | 1.00 | 27.91 | D |
| ATOM | 7740 | CA | ALA D | 531 | 146.077 | 7.470 | 53.983 | 1.00 | 25.52 | D |
| ATOM | 7741 | CB | ALA D | 531 | 145.322 | 6.506 | 54.863 | 1.00 | 23.17 | D |
| ATOM | 7742 | C | ALA D | 531 | 145.634 | 7.329 | 52.528 | 1.00 | 25.05 | D |
| ATOM | 7743 | O | ALA D | 531 | 145.166 | 8.283 | 51.903 | 1.00 | 25.19 | D |
| ATOM | 7744 | N | SER D | 532 | 145.778 | 6.126 | 52.004 | 1.00 | 24.01 | D |
| ATOM | 7745 | CA | SER D | 532 | 145.431 | 5.834 | 50.635 | 1.00 | 23.62 | D |
| ATOM | 7746 | CB | SER D | 532 | 145.452 | 4.332 | 50.427 | 1.00 | 23.66 | D |
| ATOM | 7747 | OG | SER D | 532 | 145.044 | 4.009 | 49.120 | 1.00 | 24.72 | D |
| ATOM | 7748 | C | SER D | 532 | 144.093 | 6.391 | 50.161 | 1.00 | 24.24 | D |
| ATOM | 7749 | O | SER D | 532 | 143.059 | 6.249 | 50.835 | 1.00 | 23.67 | D |
| ATOM | 7750 | N | VAL D | 533 | 144.131 | 7.028 | 48.987 | 1.00 | 22.26 | D |
| ATOM | 7751 | CA | VAL D | 533 | 142.930 | 7.588 | 48.383 | 1.00 | 21.80 | D |
| ATOM | 7752 | CB | VAL D | 533 | 143.238 | 8.790 | 47.509 | 1.00 | 19.37 | D |
| ATOM | 7753 | CG1 | VAL D | 533 | 141.973 | 9.225 | 46.786 | 1.00 | 19.14 | D |
| ATOM | 7754 | CG2 | VAL D | 533 | 143.757 | 9.912 | 48.367 | 1.00 | 16.33 | D |
| ATOM | 7755 | C | VAL D | 533 | 142.311 | 6.476 | 47.564 | 1.00 | 23.09 | D |
| ATOM | 7756 | O | VAL D | 533 | 142.740 | 6.181 | 46.464 | 1.00 | 24.67 | D |
| ATOM | 7757 | N | ARG D | 534 | 141.281 | 5.865 | 48.125 | 1.00 | 23.37 | D |
| ATOM | 7758 | CA | ARG D | 534 | 140.630 | 4.734 | 47.514 | 1.00 | 21.94 | D |
| ATOM | 7759 | CB | ARG D | 534 | 139.887 | 3.966 | 48.588 | 1.00 | 22.94 | D |
| ATOM | 7760 | CG | ARG D | 534 | 139.334 | 2.660 | 48.104 | 1.00 | 20.89 | D |
| ATOM | 7761 | CD | ARG D | 534 | 140.460 | 1.735 | 47.688 | 1.00 | 20.13 | D |
| ATOM | 7762 | NE | ARG D | 534 | 139.934 | 0.404 | 47.470 | 1.00 | 22.05 | D |
| ATOM | 7763 | CZ | ARG D | 534 | 139.177 | 0.061 | 46.435 | 1.00 | 21.88 | D |
| ATOM | 7764 | NH1 | ARG D | 534 | 138.859 | 0.955 | 45.499 | 1.00 | 16.38 | D |
| ATOM | 7765 | NH2 | ARG D | 534 | 138.723 | −1.184 | 46.354 | 1.00 | 21.24 | D |
| ATOM | 7766 | C | ARG D | 534 | 139.687 | 4.934 | 46.350 | 1.00 | 21.77 | D |
| ATOM | 7767 | O | ARG D | 534 | 139.630 | 4.082 | 45.464 | 1.00 | 23.87 | D |
| ATOM | 7768 | N | THR | 0535 | 138.944 | 6.029 | 46.328 | 1.00 | 21.46 | D |
| ATOM | 7769 | CA | THR D | 535 | 137.976 | 6.186 | 45.252 | 1.00 | 23.21 | D |
| ATOM | 7770 | CB | THR D | 535 | 136.630 | 5.607 | 45.693 | 1.00 | 22.72 | D |
| ATOM | 7771 | OG1 | THR D | 535 | 135.615 | 5.908 | 44.728 | 1.00 | 24.25 | D |
| ATOM | 7772 | CG2 | THR D | 535 | 136.255 | 6.189 | 47.038 | 1.00 | 21.59 | D |
| ATOM | 7773 | C | THR D | 535 | 137.785 | 7.623 | 44.871 | 1.00 | 22.36 | D |
| ATOM | 7774 | O | THR D | 535 | 138.010 | 8.494 | 45.692 | 1.00 | 22.27 | D |
| ATOM | 7775 | N | VAL D | 536 | 137.383 | 7.869 | 43.627 | 1.00 | 23.47 | D |
| ATOM | 7776 | CA VAL D | 536 | 137.154 | 9.240 | 43.178 | 1.00 | 26.56 | D | |
| ATOM | 7777 | CB | VAL D | 536 | 138.340 | 9.805 | 42.381 | 1.00 | 27.30 | D |
| ATOM | 7778 | CG1 | VAL D | 536 | 138.152 | 11.303 | 42.178 | 1.00 | 27.76 | D |
| ATOM | 7779 | CG2 | VAL D | 536 | 139.615 | 9.573 | 43.126 | 1.00 | 29.04 | D |
| ATOM | 7780 | C | VAL D | 536 | 135.907 | 9.379 | 42.330 | 1.00 | 26.74 | D |
| ATOM | 7781 | O | VAL D | 536 | 135.550 | 8.486 | 41.558 | 1.00 | 28.31 | D |
| ATOM | 7782 | N | SER D | 537 | 135.248 | 10.517 | 42.473 | 1.00 | 26.29 | D |
| ATOM | 7783 | CA | SER D | 537 | 134.019 | 10.759 | 41.741 | 1.00 | 25.58 | D |
| ATOM | 7784 | CB | SER D | 537 | 132.832 | 10.197 | 42.529 | 1.00 | 23.69 | D |
| ATOM | 7785 | OC | SER D | 537 | 131.605 | 10.436 | 41.878 | 1.00 | 19.22 | D |
| ATOM | 7786 | C | SER D | 537 | 133.859 | 12.252 | 41.547 | 1.00 | 26.69 | D |
| ATOM | 7787 | O | SER D | 537 | 133.697 | 13.000 | 42.517 | 1.00 | 27.22 | D |
| ATOM | 7788 | N | GLY D | 538 | 133.923 | 12.684 | 40.294 | 1.00 | 26.82 | D |
| ATOM | 7789 | CA | GLY D | 538 | 133.794 | 14.096 | 40.020 | 1.00 | 28.64 | D |
| ATOM | 7790 | C | GLY D | 538 | 133.027 | 14.446 | 38.766 | 1.00 | 28.83 | D |
| ATOM | 7791 | O | GLY D | 538 | 132.961 | 13.666 | 37.820 | 1.00 | 29.17 | D |
| ATOM | 7792 | N | HIS D | 539 | 132.450 | 15.645 | 38.790 | 1.00 | 29.15 | D |
| ATOM | 7793 | CA | HIS D | 539 | 131.679 | 16.226 | 37.696 | 1.00 | 27.88 | D |
| ATOM | 7794 | CB | HIS D | 539 | 130.179 | 16.025 | 37.911 | 1.00 | 27.43 | D |
| ATOM | 7795 | CG | HIS D | 539 | 129.333 | 16.567 | 36.799 | 1.00 | 28.11 | D |
| ATOM | 7796 | CD2 | HIS D | 539 | 128.736 | 17.772 | 36.637 | 1.00 | 30.46 | D |
| ATOM | 7797 | ND1 | HIS D | 539 | 129.046 | 15.843 | 35.662 | 1.00 | 27.67 | D |
| ATOM | 7798 | CE1 | HIS D | 539 | 128.307 | 16.577 | 34.848 | 1.00 | 29.05 | D |
| ATOM | 7799 | NE2 | HIS D | 539 | 128.105 | 17.753 | 35.415 | 1.00 | 30.92 | D |
| ATOM | 7800 | C | HIS D | 539 | 131.959 | 17.735 | 37.697 | 1.00 | 29.12 | D |
| ATOM | 7801 | O | HIS D | 539 | 131.791 | 18.409 | 38.728 | 1.00 | 28.57 | D |
| ATOM | 7802 | N | GLY D | 540 | 132.376 | 18.261 | 36.546 | 1.00 | 28.54 | D |
| ATOM | 7803 | CA | GLY D | 540 | 132.650 | 19.682 | 36.436 | 1.00 | 27.08 | D |
| ATOM | 7804 | C | GLY D | 540 | 133.872 | 20.122 | 37.215 | 1.00 | 27.09 | D |
| ATOM | 7805 | O | GLY D | 540 | 134.904 | 19.454 | 37.179 | 1.00 | 28.54 | D |
| ATOM | 7806 | N | ASN D | 541 | 133.777 | 21.245 | 37.920 | 1.00 | 25.16 | D |
| ATOM | 7807 | CA | ASN D | 541 | 134.924 | 21.706 | 38.679 | 1.00 | 24.86 | D |
| ATOM | 7808 | CB | ASN D | 541 | 134.907 | 23.237 | 38.816 | 1.00 | 26.35 | D |
| ATOM | 7809 | CG | ASN D | 541 | 133.889 | 23.726 | 39.851 | 1.00 | 29.47 | D |
| ATOM | 7810 | OD1 | ASN D | 541 | 132.706 | 23.341 | 39.803 | 1.00 | 29.21 | D |
| ATOM | 7811 | ND2 | ASN D | 541 | 134.341 | 24.575 | 40.792 | 1.00 | 26.35 | D |
| ATOM | 7812 | C | ASN D | 541 | 134.915 | 21.062 | 40.056 | 1.00 | 24.63 | D |
| ATOM | 7813 | O | ASN D | 541 | 135.803 | 21.308 | 40.864 | 1.00 | 25.54 | D |
| ATOM | 7814 | N | ILE D | 542 | 133.921 | 20.220 | 40.321 | 1.00 | 22.93 | D |
| ATOM | 7815 | CA | ILE D | 542 | 133.821 | 19.574 | 41.629 | 1.00 | 22.46 | D |
| ATOM | 7816 | CB | ILE D | 542 | 132.361 | 19.724 | 42.186 | 1.00 | 20.91 | D |
| ATOM | 7817 | CG2 | ILE D | 542 | 132.215 | 18.977 | 43.497 | 1.00 | 22.05 | D |
| ATOM | 7818 | CG1 | ILE D | 542 | 132.001 | 21.215 | 42.385 | 1.00 | 19.70 | D |
| ATOM | 7819 | CD1 | ILE D | 542 | 132.866 | 22.016 | 43.388 | 1.00 | 10.85 | D |
| ATOM | 7820 | C | ILE D | 542 | 134.258 | 18.085 | 41.663 | 1.00 | 24.27 | D |
| ATOM | 7821 | O | ILE D | 542 | 133.841 | 17.258 | 40.832 | 1.00 | 23.68 | D |
| ATOM | 7822 | N | VAL D | 543 | 135.108 | 17.738 | 42.624 | 1.00 | 24.95 | D |
| ATOM | 7823 | CA | VAL D | 543 | 135.565 | 16.350 | 42.735 | 1.00 | 26.33 | D |
| ATOM | 7824 | CB | VAL D | 543 | 137.023 | 16.176 | 42.199 | 1.00 | 28.00 | D |
| ATOM | 7825 | CG1 | VAL D | 543 | 137.492 | 14.742 | 42.406 | 1.00 | 27.16 | D |
| ATOM | 7826 | CG2 | VAL D | 543 | 137.073 | 16.515 | 40.716 | 1.00 | 30.18 | D |
| ATOM | 7827 | C | VAL D | 543 | 135.509 | 15.864 | 44.174 | 1.00 | 25.02 | D |
| ATOM | 7828 | O | VAL D | 543 | 135.893 | 16.601 | 45.079 | 1.00 | 24.45 | D |
| ATOM | 7829 | N | VAL D | 544 | 135.017 | 14.639 | 44.388 | 1.00 | 24.93 | D |
| ATOM | 7830 | CA | VAL D | 544 | 134.939 | 14.093 | 45.748 | 1.00 | 25.31 | D |
| ATOM | 7831 | CB | VAL D | 544 | 133.495 | 13.783 | 46.197 | 1.00 | 24.60 | D |
| ATOM | 7832 | CG1 | VAL D | 544 | 133.507 | 13.509 | 47.679 | 1.00 | 24.71 | D |
| ATOM | 7833 | CG2 | VAL D | 544 | 132.553 | 14.940 | 45.889 | 1.00 | 25.25 | D |
| ATOM | 7834 | C | VAL D | 544 | 135.750 | 12.814 | 45.910 | 1.00 | 25.25 | D |
| ATOM | 7835 | O | VAL D | 544 | 135.662 | 11.903 | 45.082 | 1.00 | 26.67 | D |
| ATOM | 7836 | N | SER D | 545 | 136.534 | 12.745 | 46.985 | 1.00 | 24.15 | D |
| ATOM | 7837 | CA | SER D | 545 | 137.367 | 11.566 | 47.236 | 1.00 | 24.66 | D |
| ATOM | 7838 | CB | SER D | 545 | 138.835 | 11.960 | 47.213 | 1.00 | 22.85 | D |
| ATOM | 7839 | OG | SER D | 545 | 139.110 | 12.914 | 48.221 | 1.00 | 26.17 | D |
| ATOM | 7840 | C | SER D | 545 | 137.105 | 10.777 | 48.525 | 1.00 | 26.03 | D |
| ATOM | 7841 | O | SER D | 545 | 136.609 | 11.315 | 49.516 | 1.00 | 27.76 | D |
| ATOM | 7842 | N | GLY D | 546 | 137.467 | 9.497 | 48.495 | 1.00 | 25.26 | D |
| ATOM | 7843 | CA | GLY D | 546 | 137.292 | 8.642 | 49.647 | 1.00 | 24.44 | D |
| ATOM | 7844 | C | GLY D | 546 | 138.637 | 8.073 | 50.039 | 1.00 | 24.54 | D |
| ATOM | 7845 | O | GLY D | 546 | 139.318 | 7.450 | 49.220 | 1.00 | 23.24 | D |
| ATOM | 7846 | N | SER D | 547 | 139.038 | 8.282 | 51.288 | 1.00 | 25.20 | D |
| ATOM | 7847 | CA | SER D | 547 | 140.339 | 7.773 | 51.730 | 1.00 | 27.40 | D |
| ATOM | 7848 | CB | SER D | 547 | 141.235 | 8.897 | 52.239 | 1.00 | 25.06 | D |
| ATOM | 7849 | OG | SER D | 547 | 142.424 | 8.333 | 52.763 | 1.00 | 23.13 | D |
| ATOM | 7850 | C | SER D | 547 | 140.286 | 6.723 | 52.816 | 1.00 | 28.75 | D |
| ATOM | 7851 | O | SER D | 547 | 139.260 | 6.520 | 53.462 | 1.00 | 32.39 | D |
| ATOM | 7852 | N | TYR D | 548 | 141.411 | 6.062 | 53.031 | 1.00 | 27.35 | D |
| ATOM | 7853 | CA | TYR D | 548 | 141.457 | 5.071 | 54.078 | 1.00 | 24.34 | D |
| ATOM | 7854 | CB | TYR D | 548 | 142.588 | 4.085 | 53.835 | 1.00 | 21.23 | D |
| ATOM | 7855 | CG | TYR D | 548 | 142.185 | 2.933 | 52.964 | 1.00 | 18.24 | D |
| ATOM | 7856 | CD1 | TYR D | 548 | 140.975 | 2.940 | 52.267 | 1.00 | 17.37 | D |
| ATOM | 7857 | CE1 | TYR D | 548 | 140.631 | 1.894 | 51.426 | 1.00 | 15.22 | D |
| ATOM | 7858 | CD2 | TYR D | 548 | 143.030 | 1.850 | 52.802 | 1.00 | 18.07 | D |
| ATOM | 7859 | CE2 | TYR D | 548 | 142.701 | 0.796 | 51.969 | 1.00 | 15.33 | D |
| ATOM | 7860 | CZ | TYR D | 548 | 141.507 | 0.825 | 51.283 | 1.00 | 17.35 | D |
| ATOM | 7861 | OH | TYR D | 548 | 141.195 | −0.213 | 50.435 | 1.00 | 18.41 | D |
| ATOM | 7862 | C | TYR D | 548 | 141.637 | 5.742 | 55.428 | 1.00 | 25.52 | D |
| ATOM | 7863 | O | TYR D | 548 | 141.897 | 5.060 | 56.401 | 1.00 | 24.80 | D |
| ATOM | 7864 | N | ASP D | 549 | 141.517 | 7.070 | 55.496 | 1.00 | 26.28 | D |
| ATOM | 7865 | CA | ASP D | 549 | 141.637 | 7.739 | 56.789 | 1.00 | 26.66 | D |
| ATOM | 7866 | CB | ASP D | 549 | 142.332 | 9.114 | 56.693 | 1.00 | 28.11 | D |
| ATOM | 7867 | CG | ASP D | 549 | 141.761 | 10.006 | 55.611 | 1.00 | 28.18 | D |
| ATOM | 7868 | OD1 | ASP D | 549 | 140.580 | 9.822 | 55.227 | 1.00 | 30.65 | D |
| ATOM | 7869 | OD2 | ASP D | 549 | 142.507 | 10.909 | 55.167 | 1.00 | 25.41 | D |
| ATOM | 7870 | C | ASP D | 549 | 140.239 | 7.884 | 57.363 | 1.00 | 26.46 | D |
| ATOM | 7871 | O | ASP D | 549 | 140.014 | 8.634 | 58.311 | 1.00 | 26.79 | D |
| ATOM | 7872 | N | ASN D | 550 | 139.309 | 7.157 | 56.742 | 1.00 | 26.14 | D |
| ATOM | 7873 | CA | ASN D | 550 | 137.906 | 7.115 | 57.139 | 1.00 | 24.53 | D |
| ATOM | 7874 | CB | ASN D | 550 | 137.825 | 6.849 | 58.633 | 1.00 | 23.45 | D |
| ATOM | 7875 | CG | ASN D | 550 | 138.484 | 5.542 | 59.012 | 1.00 | 23.88 | D |
| ATOM | 7876 | OD1 | ASN D | 550 | 139.126 | 4.894 | 58.190 | 1.00 | 24.66 | D |
| ATOM | 7877 | ND2 | ASN D | 550 | 138.319 | 5.143 | 60.253 | 1.00 | 25.10 | D |
| ATOM | 7878 | C | ASN D | 550 | 137.162 | 8.374 | 56.796 | 1.00 | 23.51 | D |
| ATOM | 7879 | O | ASN D | 550 | 136.006 | 8.534 | 57.146 | 1.00 | 23.99 | D |
| ATOM | 7880 | N | THR D | 551 | 137.811 | 9.237 | 56.038 | 1.00 | 24.61 | D |
| ATOM | 7881 | CA | THR D | 551 | 137.221 | 10.510 | 55.706 | 1.00 | 24.98 | D |
| ATOM | 7882 | CB | THR D | 551 | 138.155 | 11.584 | 56.208 | 1.00 | 25.84 | D |
| ATOM | 7883 | OG1 | THR D | 551 | 137.402 | 12.640 | 56.803 | 1.00 | 26.76 | D |
| ATOM | 7884 | CG2 | THR D | 551 | 139.015 | 12.107 | 55.053 | 1.00 | 24.97 | D |
| ATOM | 7885 | C | THR D | 551 | 136.993 | 10.725 | 54.206 | 1.00 | 25.92 | D |
| ATOM | 7886 | O | THR D | 551 | 137.535 | 9.996 | 53.376 | 1.00 | 24.99 | D |
| ATOM | 7887 | N | LEU D | 552 | 136.173 | 11.719 | 53.868 | 1.00 | 26.68 | D |
| ATOM | 7888 | CA | LEU D | 552 | 135.934 | 12.079 | 52.471 | 1.00 | 26.79 | D |
| ATOM | 7889 | CB | LEU D | 552 | 134.508 | 11.771 | 52.031 | 1.00 | 24.86 | D |
| ATOM | 7890 | CG | LEU D | 552 | 134.189 | 10.328 | 51.665 | 1.00 | 23.77 | D |
| ATOM | 7891 | CD1 | LEU D | 552 | 133.215 | 9.804 | 52.689 | 1.00 | 23.78 | D |
| ATOM | 7892 | CD2 | LEU D | 552 | 133.597 | 10.237 | 50.257 | 1.00 | 22.54 | D |
| ATOM | 7893 | C | LEU D | 552 | 136.180 | 13.572 | 52.308 | 1.00 | 27.51 | D |
| ATOM | 7894 | O | LEU D | 552 | 135.792 | 14.366 | 53.170 | 1.00 | 28.40 | D |
| ATOM | 7895 | N | ILE D | 553 | 136.828 | 13.955 | 51.206 | 1.00 | 27.13 | D |
| ATOM | 7896 | CA | ILE D | 553 | 137.102 | 15.372 | 50.939 | 1.00 | 25.65 | D |
| ATOM | 7897 | CE | ILE D | 553 | 138.623 | 15.686 | 50.957 | 1.00 | 24.82 | D |
| ATOM | 7898 | CG2 | ILE D | 553 | 138.854 | 17.153 | 50.606 | 1.00 | 24.33 | D |
| ATOM | 7899 | CG1 | ILE D | 553 | 139.212 | 15.444 | 52.355 | 1.00 | 25.26 | D |
| ATOM | 7900 | CD1 | ILE D | 553 | 140.725 | 15.613 | 52.413 | 1.00 | 24.18 | D |
| ATOM | 7901 | C | ILE D | 553 | 136.531 | 15.869 | 49.606 | 1.00 | 25.82 | D |
| ATOM | 7902 | O | ILE D | 553 | 136.664 | 15.210 | 48.561 | 1.00 | 24.35 | D |
| ATOM | 7903 | N | VAL D | 554 | 135.889 | 17.038 | 49.672 | 1.00 | 24.96 | D |
| ATOM | 7904 | CA | VAL D | 554 | 135.299 | 17.704 | 48.503 | 1.00 | 24.79 | D |
| ATOM | 7905 | CB | VAL D | 554 | 133.964 | 18.425 | 48.858 | 1.00 | 21.69 | D |
| ATOM | 7906 | CG1 | VAL D | 554 | 133.366 | 19.041 | 47.620 | 1.00 | 19.43 | D |
| ATOM | 7907 | CG2 | VAL D | 554 | 133.000 | 17.455 | 49.480 | 1.00 | 21.35 | D |
| ATOM | 7908 | C | VAL D | 554 | 136.308 | 18.753 | 48.016 | 1.00 | 26.46 | D |
| ATOM | 7909 | O | VAL D | 554 | 136.646 | 19.689 | 48.746 | 1.00 | 26.64 | D |
| ATOM | 7910 | N | TRP D | 555 | 136.812 | 18.574 | 46.797 | 1.00 | 27.69 | D |
| ATOM | 7911 | CA | TRP D | 555 | 137.784 | 19.500 | 46.233 | 1.00 | 28.09 | D |
| ATOM | 7912 | CB | TRP D | 555 | 139.026 | 18.767 | 45.703 | 1.00 | 28.18 | D |
| ATOM | 7913 | CG | TRP D | 555 | 139.593 | 17.681 | 46.576 | 1.00 | 29.63 | D |
| ATOM | 7914 | CD2 | TRP D | 555 | 140.695 | 17.795 | 47.487 | 1.00 | 30.60 | D |
| ATOM | 7915 | CE2 | TRP D | 555 | 140.948 | 16.505 | 48.003 | 1.00 | 30.79 | D |
| ATOM | 7916 | CE3 | TRP D | 555 | 141.492 | 18.862 | 47.915 | 1.00 | 30.24 | D |
| ATOM | 7917 | CD1 | TRP D | 555 | 139.228 | 16.365 | 46.587 | 1.00 | 29.55 | D |
| ATOM | 7918 | NE1 | TRP D | 555 | 140.041 | 15.652 | 47.436 | 1.00 | 30.90 | D |
| ATOM | 7919 | CZ2 | TRP D | 555 | 141.968 | 16.253 | 48.921 | 1.00 | 31.50 | D |
| ATOM | 7920 | CZ3 | TRP D | 555 | 142.510 | 18.608 | 48.832 | 1.00 | 31.00 | D |
| ATOM | 7921 | CH2 | TRP D | 555 | 142.736 | 17.314 | 49.323 | 1.00 | 31.35 | D |
| ATOM | 7922 | C | TRP D | 555 | 137.210 | 20.306 | 45.075 | 1.00 | 27.75 | D |
| ATOM | 7923 | O | TRP D | 555 | 136.392 | 19.807 | 44.297 | 1.00 | 26.08 | D |
| ATOM | 7924 | N | ASP D | 556 | 137.658 | 21.556 | 44.981 | 1.00 | 29.07 | D |
| ATOM | 7925 | CA | ASP D | 556 | 137.274 | 22.463 | 43.904 | 1.00 | 29.82 | D |
| ATOM | 7926 | CB | ASP D | 556 | 136.917 | 23.849 | 44.425 | 1.00 | 33.43 | D |
| ATOM | 7927 | CG | ASP D | 556 | 136.336 | 24.762 | 43.335 | 1.00 | 36.51 | D |
| ATOM | 7928 | OD1 | ASP D | 556 | 136.913 | 24.864 | 42.216 | 1.00 | 35.80 | D |
| ATOM | 7929 | OD2 | ASP D | 556 | 135.285 | 25.388 | 43.609 | 1.00 | 39.49 | D |
| ATOM | 7930 | C | ASP D | 556 | 138.539 | 22.591 | 43.095 | 1.00 | 28.83 | D |
| ATOM | 7931 | O | ASP D | 556 | 139.440 | 23.337 | 43.463 | 1.00 | 25.39 | D |
| ATOM | 7932 | N | VAL D | 557 | 138.596 | 21.858 | 41.995 | 1.00 | 31.59 | D |
| ATOM | 7933 | CA | VAL D | 557 | 139.756 | 21.853 | 41.123 | 1.00 | 34.16 | D |
| ATOM | 7934 | CE | VAL D | 557 | 139.592 | 20.840 | 40.007 | 1.00 | 34.22 | D |
| ATOM | 7935 | CG1 | VAL D | 557 | 140.777 | 20.942 | 39.064 | 1.00 | 36.74 | D |
| ATOM | 7936 | CG2 | VAL D | 557 | 139.460 | 19.444 | 40.582 | 1.00 | 31.24 | D |
| ATOM | 7937 | C | VAL D | 557 | 140.021 | 23.183 | 40.466 | 1.00 | 35.74 | D |
| ATOM | 7938 | O | VAL D | 557 | 141.168 | 23.535 | 40.244 | 1.00 | 37.94 | D |
| ATOM | 7939 | N | ALA D | 558 | 138.959 | 23.906 | 40.133 | 1.00 | 37.02 | D |
| ATOM | 7940 | CA | ALA D | 558 | 139.107 | 25.196 | 39.485 | 1.00 | 37.13 | D |
| ATOM | 7941 | CB | ALA D | 558 | 137.747 | 25.730 | 39.065 | 1.00 | 39.25 | D |
| ATOM | 7942 | C | ALA D | 558 | 139.784 | 26.167 | 40.430 | 1.00 | 36.58 | D |
| ATOM | 7943 | O | ALA D | 558 | 140.342 | 27.168 | 39.997 | 1.00 | 37.02 | D |
| ATOM | 7944 | N | GLN D | 559 | 139.729 | 25.857 | 41.722 | 1.00 | 37.85 | D |
| ATOM | 7945 | CA | GLN D | 559 | 140.327 | 26.688 | 42.763 | 1.00 | 37.56 | D |
| ATOM | 7946 | CB | GLN D | 559 | 139.248 | 27.110 | 43.750 | 1.00 | 37.90 | D |
| ATOM | 7947 | CG | GLN D | 559 | 139.672 | 28.264 | 44.624 | 1.00 | 43.44 | D |
| ATOM | 7948 | CD | GLN D | 559 | 138.523 | 28.874 | 45.444 | 1.00 | 45.28 | D |
| ATOM | 7949 | OE1 | GLN D | 559 | 137.375 | 28.927 | 44.987 | 1.00 | 46.49 | D |
| ATOM | 7950 | NE2 | GLN D | 559 | 138.839 | 29.354 | 46.655 | 1.00 | 45.82 | D |
| ATOM | 7951 | C | GLN D | 559 | 141.419 | 25.896 | 43.479 | 1.00 | 37.70 | D |
| ATOM | 7952 | O | GLN D | 559 | 142.131 | 26.428 | 44.336 | 1.00 | 36.85 | D |
| ATOM | 7953 | N | MSE D | 560 | 141.535 | 24.624 | 43.094 | 1.00 | 38.22 | D |
| ATOM | 7954 | CA | MSE D | 560 | 142.505 | 23.687 | 43.645 | 1.00 | 37.94 | D |
| ATOM | 7955 | CB | MSE D | 560 | 143.870 | 23.912 | 42.984 | 1.00 | 37.36 | D |
| ATOM | 7956 | CG | MSE D | 560 | 144.681 | 22.648 | 42.719 | 1.00 | 37.70 | D |
| ATOM | 7957 | SE | MSE D | 560 | 143.786 | 21.540 | 41.377 | 1.00 | 39.17 | D |
| ATOM | 7958 | CE | MSE D | 560 | 143.835 | 22.738 | 39.886 | 1.00 | 41.26 | D |
| ATOM | 7959 | C | MSE D | 560 | 142.597 | 23.865 | 45.164 | 1.00 | 37.81 | D |
| ATOM | 7960 | O | MSE D | 560 | 143.675 | 24.035 | 45.710 | 1.00 | 39.53 | D |
| ATOM | 7961 | N | LYS D | 561 | 141.449 | 23.844 | 45.832 | 1.00 | 37.74 | D |
| ATOM | 7962 | CA | LYS D | 561 | 141.384 | 23.995 | 47.279 | 1.00 | 38.73 | D |
| ATOM | 7963 | CB | LYS D | 561 | 141.037 | 25.452 | 47.638 | 1.00 | 39.81 | D |
| ATOM | 7964 | CG | LYS D | 561 | 142.191 | 26.428 | 47.399 | 0.00 | 40.07 | D |
| ATOM | 7965 | CD | LYS D | 561 | 143.372 | 26.124 | 48.318 | 0.00 | 40.65 | D |
| ATOM | 7966 | CE | LYS D | 561 | 142.998 | 26.355 | 49.774 | 0.00 | 41.01 | D |
| ATOM | 7967 | NZ | LYS D | 561 | 144.024 | 25.827 | 50.715 | 0.00 | 41.29 | D |
| ATOM | 7968 | C | LYS D | 561 | 140.356 | 23.022 | 47.887 | 1.00 | 39.22 | D |
| ATOM | 7969 | O | LYS D | 561 | 139.419 | 22.584 | 47.208 | 1.00 | 38.84 | D |
| ATOM | 7970 | N | CYS D | 562 | 140.556 | 22.670 | 49.159 | 1.00 | 38.90 | D |
| ATOM | 7971 | CA | CYS D | 562 | 139.668 | 21.759 | 49.885 | 1.00 | 38.52 | D |
| ATOM | 7972 | CB | CYS D | 562 | 140.360 | 21.270 | 51.164 | 1.00 | 38.67 | D |
| ATOM | 7973 | SG | CYS D | 562 | 139.309 | 20.275 | 52.277 | 1.00 | 44.51 | D |
| ATOM | 7974 | C | CYS D | 562 | 138.402 | 22.521 | 50.260 | 1.00 | 37.58 | D |
| ATOM | 7975 | O | CYS D | 562 | 138.494 | 23.572 | 50.888 | 1.00 | 40.09 | D |
| ATOM | 7976 | N | LEU D | 563 | 137.223 | 22.015 | 49.895 | 1.00 | 34.69 | D |
| ATOM | 7977 | CA | LEU D | 563 | 135.981 | 22.726 | 50.232 | 1.00 | 31.60 | D |
| ATOM | 7978 | CB | LEU D | 563 | 134.924 | 22.550 | 49.132 | 1.00 | 28.39 | D |
| ATOM | 7979 | CG | LEU D | 563 | 135.214 | 23.279 | 47.820 | 1.00 | 25.92 | D |
| ATOM | 7980 | CD1 | LEU D | 563 | 133.986 | 23.317 | 46.939 | 1.00 | 24.65 | D |
| ATOM | 7981 | CD2 | LEU D | 563 | 135.679 | 24.670 | 48.139 | 1.00 | 23.99 | D |
| ATOM | 7982 | C | LEU D | 563 | 135.388 | 22.281 | 51.552 | 1.00 | 30.82 | D |
| ATOM | 7983 | O | LEU D | 563 | 135.012 | 23.099 | 52.388 | 1.00 | 29.72 | D |
| ATOM | 7984 | N | TYR D | 564 | 135.320 | 20.968 | 51.723 | 1.00 | 31.20 | D |
| ATOM | 7985 | CA | TYR D | 564 | 134.768 | 20.358 | 52.915 | 1.00 | 29.79 | D |
| ATOM | 7986 | CB | TYR D | 564 | 133.276 | 20.087 | 52.705 | 1.00 | 31.53 | D |
| ATOM | 7987 | CG | TYR D | 564 | 132.415 | 21.316 | 52.480 | 1.00 | 35.18 | D |
| ATOM | 7988 | CD1 | TYR D | 564 | 132.216 | 22.250 | 53.503 | 1.00 | 35.47 | D |
| ATOM | 7989 | CE1 | TYR D | 564 | 131.381 | 23.356 | 53.321 | 1.00 | 37.11 | D |
| ATOM | 7990 | CD2 | TYR D | 564 | 131.757 | 21.522 | 51.257 | 1.00 | 35.79 | D |
| ATOM | 7991 | CE2 | TYR D | 564 | 130.918 | 22.626 | 51.064 | 1.00 | 37.32 | D |
| ATOM | 7992 | CZ | TYR D | 564 | 130.733 | 23.539 | 52.100 | 1.00 | 37.56 | D |
| ATOM | 7993 | OH | TYR D | 564 | 129.901 | 24.625 | 51.926 | 1.00 | 37.99 | D |
| ATOM | 7994 | C | TYR D | 564 | 135.464 | 19.028 | 53.213 | 1.00 | 28.42 | D |
| ATOM | 7995 | O | TYR D | 564 | 135.998 | 18.357 | 52.323 | 1.00 | 29.93 | D |
| ATOM | 7996 | N | ILE D | 565 | 135.448 | 18.655 | 54.482 | 1.00 | 26.22 | D |
| ATOM | 7997 | CA | ILE D | 565 | 136.001 | 17.389 | 54.916 | 1.00 | 24.23 | D |
| ATOM | 7998 | CB | ILE D | 565 | 137.222 | 17.574 | 55.861 | 1.00 | 24.90 | D |
| ATOM | 7999 | CG2 | ILE D | 565 | 137.641 | 16.232 | 56.471 | 1.00 | 22.64 | D |
| ATOM | 8000 | CG1 | ILE D | 565 | 138.389 | 18.173 | 55.066 | 1.00 | 23.83 | D |
| ATOM | 8001 | CD1 | ILE D | 565 | 139.596 | 18.476 | 55.898 | 1.00 | 22.10 | D |
| ATOM | 8002 | C | ILE D | 565 | 134.853 | 16.708 | 55.654 | 1.00 | 24.14 | D |
| ATOM | 8003 | O | ILE D | 565 | 134.436 | 17.123 | 56.745 | 1.00 | 24.92 | D |
| ATOM | 8004 | N | LEU D | 566 | 134.319 | 15.674 | 55.022 | 1.00 | 23.68 | D |
| ATOM | 8005 | CA | LEU D | 566 | 133.209 | 14.910 | 55.567 | 1.00 | 21.44 | D |
| ATOM | 8006 | CB | LEU D | 566 | 132.498 | 14.204 | 54.433 | 1.00 | 18.60 | D |
| ATOM | 8007 | CG | LEU D | 566 | 132.249 | 15.108 | 53.240 | 1.00 | 16.04 | D |
| ATOM | 8008 | CD1 | LEU D | 566 | 131.493 | 14.312 | 52.195 | 1.00 | 18.64 | D |
| ATOM | 8009 | CD2 | LEU D | 566 | 131.471 | 16.327 | 53.661 | 1.00 | 11.95 | D |
| ATOM | 8010 | C | LEU D | 566 | 133.725 | 13.876 | 56.532 | 1.00 | 20.93 | D |
| ATOM | 8011 | O | LEU D | 566 | 134.448 | 12.975 | 56.126 | 1.00 | 21.77 | D |
| ATOM | 8012 | N | SER D | 567 | 133.345 | 13.980 | 57.800 | 1.00 | 22.31 | D |
| ATOM | 8013 | CA | SER D | 567 | 133.806 | 12.995 | 58.783 | 1.00 | 23.94 | D |
| ATOM | 8014 | CB | SER D | 567 | 134.810 | 13.630 | 59.731 | 1.00 | 23.34 | D |
| ATOM | 8015 | OG | SER D | 567 | 135.808 | 14.311 | 58.987 | 1.00 | 24.18 | D |
| ATOM | 8016 | C | SER D | 567 | 132.676 | 12.349 | 59.568 | 1.00 | 24.28 | D |
| ATOM | 8017 | O | SER D | 567 | 131.586 | 12.904 | 59.663 | 1.00 | 24.81 | D |
| ATOM | 8018 | N | GLY D | 568 | 132.950 | 11.166 | 60.113 | 1.00 | 25.74 | D |
| ATOM | 8019 | CA | GLY D | 568 | 131.953 | 10.416 | 60.858 | 1.00 | 25.21 | D |
| ATOM | 8020 | C | GLY D | 568 | 132.134 | 8.906 | 60.681 | 1.00 | 26.37 | D |
| ATOM | 8021 | O | GLY D | 568 | 132.130 | 8.163 | 61.664 | 1.00 | 27.73 | D |
| ATOM | 8022 | N | HIS D | 569 | 132.288 | 8.440 | 59.437 | 1.00 | 25.07 | D |
| ATOM | 8023 | CA | HIS D | 569 | 132.500 | 7.007 | 59.175 | 1.00 | 23.33 | D |
| ATOM | 8024 | CB | HIS D | 569 | 132.949 | 6.749 | 57.730 | 1.00 | 22.22 | D |
| ATOM | 8025 | CG | HIS D | 569 | 131.839 | 6.801 | 56.725 | 1.00 | 21.96 | D |
| ATOM | 8026 | CD2 | HIS D | 569 | 131.594 | 7.675 | 55.720 | 1.00 | 21.82 | D |
| ATOM | 8027 | ND1 | HIS D | 569 | 130.827 | 5.865 | 56.671 | 1.00 | 20.28 | D |
| ATOM | 8028 | CE1 | HIS D | 569 | 130.009 | 6.160 | 55.676 | 1.00 | 20.59 | D |
| ATOM | 8029 | NE2 | HIS D | 569 | 130.452 | 7.254 | 55.083 | 1.00 | 20.12 | D |
| ATOM | 8030 | C | HIS D | 569 | 133.590 | 6.520 | 60.129 | 1.00 | 24.25 | D |
| ATOM | 8031 | O | HIS D | 569 | 134.625 | 7.176 | 60.291 | 1.00 | 24.38 | D |
| ATOM | 8032 | N | THR D | 570 | 133.357 | 5.371 | 60.759 | 1.00 | 25.24 | D |
| ATOM | 8033 | CA | THR D | 570 | 134.296 | 4.806 | 61.724 | 1.00 | 25.54 | D |
| ATOM | 8034 | CE | THR D | 570 | 133.508 | 4.097 | 62.867 | 1.00 | 26.19 | D |
| ATOM | 8035 | OG1 | THR D | 570 | 132.385 | 3.381 | 62.321 | 1.00 | 27.58 | D |
| ATOM | 8036 | CG2 | THR D | 570 | 133.008 | 5.120 | 63.869 | 1.00 | 23.79 | D |
| ATOM | 8037 | C | THR D | 570 | 135.355 | 3.864 | 61.114 | 1.00 | 25.50 | D |
| ATOM | 8038 | O | THR D | 570 | 136.225 | 3.349 | 61.812 | 1.00 | 26.67 | D |
| ATOM | 8039 | N | ASP D | 571 | 135.279 | 3.628 | 59.811 | 1.00 | 26.22 | D |
| ATOM | 8040 | CA | ASP D | 571 | 136.284 | 2.804 | 59.141 | 1.00 | 25.18 | D |
| ATOM | 8041 | CB | ASP D | 571 | 135.810 | 1.365 | 58.999 | 1.00 | 26.72 | D |
| ATOM | 8042 | CG | ASP D | 571 | 136.956 | 0.386 | 59.009 | 1.00 | 28.41 | D |
| ATOM | 8043 | OD1 | ASP D | 571 | 138.011 | 0.726 | 58.447 | 1.00 | 30.11 | D |
| ATOM | 8044 | OD2 | ASP D | 571 | 136.818 | −0.715 | 59.575 | 1.00 | 29.31 | D |
| ATOM | 8045 | C | ASP D | 571 | 136.590 | 3.382 | 57.759 | 1.00 | 24.07 | D |
| ATOM | 8046 | O | ASP D | 571 | 135.917 | 4.303 | 57.281 | 1.00 | 22.39 | D |
| ATOM | 8047 | N | ARG D | 572 | 137.593 | 2.825 | 57.101 | 1.00 | 23.27 | D |
| ATOM | 8048 | CA | ARG D | 572 | 137.976 | 3.314 | 55.781 | 1.00 | 22.84 | D |
| ATOM | 8049 | CB | ARG D | 572 | 139.153 | 2.491 | 55.250 | 1.00 | 21.15 | D |
| ATOM | 8050 | CG | ARG D | 572 | 138.981 | 1.032 | 55.408 | 1.00 | 19.04 | D |
| ATOM | 8051 | CD | ARG D | 572 | 140.058 | 0.268 | 54.705 | 1.00 | 20.64 | D |
| ATOM | 8052 | NE | ARG D | 572 | 141.341 | 0.249 | 55.393 | 1.00 | 22.31 | D |
| ATOM | 8053 | CZ | ARG D | 572 | 142.293 | −0.649 | 55.130 | 1.00 | 23.14 | D |
| ATOM | 8054 | NH1 | ARG D | 572 | 142.071 | −1.571 | 54.198 | 1.00 | 17.78 | D |
| ATOM | 8055 | NH2 | ARG D | 572 | 143.449 | −0.646 | 55.799 | 1.00 | 23.35 | D |
| ATOM | 8056 | C | ARG D | 572 | 136.820 | 3.300 | 54.789 | 1.00 | 22.51 | D |
| ATOM | 8057 | O | ARG D | 572 | 135.914 | 2.475 | 54.922 | 1.00 | 22.42 | D |
| ATOM | 8058 | N | ILE D | 573 | 136.825 | 4.241 | 53.837 | 1.00 | 23.14 | D |
| ATOM | 8059 | CA | ILE D | 573 | 135.779 | 4.301 | 52.809 | 1.00 | 24.51 | D |
| ATOM | 8060 | CB | ILE D | 573 | 135.449 | 5.739 | 52.295 | 1.00 | 27.12 | D |
| ATOM | 8061 | CG2 | ILE D | 573 | 134.618 | 5.643 | 51.025 | 0.00 | 26.23 | D |
| ATOM | 8062 | CG1 | ILE D | 573 | 134.652 | 6.536 | 53.333 | 1.00 | 26.27 | D |
| ATOM | 8063 | CD1 | ILE D | 573 | 135.472 | 7.060 | 54.419 | 1.00 | 25.64 | D |
| ATOM | 8064 | C | ILE D | 573 | 136.330 | 3.577 | 51.601 | 1.00 | 24.11 | D |
| ATOM | 8065 | O | ILE D | 573 | 137.517 | 3.724 | 51.270 | 1.00 | 23.80 | D |
| ATOM | 8066 | N | TYR D | 574 | 135.459 | 2.830 | 50.927 | 1.00 | 22.78 | D |
| ATOM | 8067 | CA | TYR D | 574 | 135.849 | 2.071 | 49.742 | 1.00 | 22.23 | D |
| ATOM | 8068 | CB | TYR D | 574 | 135.498 | 0.583 | 49.951 | 1.00 | 21.98 | D |
| ATOM | 8069 | CG | TYR D | 574 | 136.588 | −0.242 | 50.619 | 1.00 | 24.35 | D |
| ATOM | 8070 | CD1 | TYR D | 574 | 137.503 | −0.992 | 49.853 | 1.00 | 22.37 | D |
| ATOM | 8071 | CE1 | TYR D | 574 | 138.524 | −1.729 | 50.463 | 1.00 | 23.23 | D |
| ATOM | 8072 | CD2 | TYR D | 574 | 136.723 | −0.254 | 52.019 | 1.00 | 24.83 | D |
| ATOM | 8073 | CE2 | TYR D | 574 | 137.736 | −0.982 | 52.641 | 1.00 | 26.35 | D |
| ATOM | 8074 | CZ | TYR D | 574 | 138.639 | −1.719 | 51.862 | 1.00 | 25.64 | D |
| ATOM | 8075 | OH | TYR D | 574 | 139.650 | −2.423 | 52.493 | 1.00 | 26.87 | D |
| ATOM | 8076 | C | TYR D | 574 | 135.245 | 2.570 | 48.402 | 1.00 | 21.18 | D |
| ATOM | 8077 | O | TYR D | 574 | 135.716 | 2.188 | 47.325 | 1.00 | 22.28 | D |
| ATOM | 8078 | N | SER D | 575 | 134.231 | 3.432 | 48.464 | 1.00 | 19.10 | D |
| ATOM | 8079 | CA | SER D | 575 | 133.556 | 3.933 | 47.258 | 1.00 | 18.07 | D |
| ATOM | 8080 | CB | SER D | 575 | 132.559 | 2.872 | 46.756 | 1.00 | 18.09 | D |
| ATOM | 8081 | OG | SER D | 575 | 131.552 | 3.435 | 45.926 | 1.00 | 17.37 | D |
| ATOM | 8082 | C | SER D | 575 | 132.814 | 5.255 | 47.504 | 1.00 | 17.95 | D |
| ATOM | 8083 | O | SER D | 575 | 132.312 | 5.515 | 48.599 | 1.00 | 17.19 | D |
| ATOM | 8084 | N | THR D | 576 | 132.731 | 6.105 | 46.498 | 1.00 | 17.64 | D |
| ATOM | 8085 | CA | THR D | 576 | 132.009 | 7.334 | 46.737 | 1.00 | 19.01 | D |
| ATOM | 8086 | CB | THR D | 576 | 132.936 | 8.441 | 47.285 | 1.00 | 17.27 | D |
| ATOM | 8087 | OG1 | THR D | 576 | 132.145 | 9.575 | 47.662 | 1.00 | 15.53 | D |
| ATOM | 8088 | CG2 | THR D | 576 | 133.968 | 8.869 | 46.228 | 1.00 | 20.28 | D |
| ATOM | 8089 | C | THR D | 576 | 131.369 | 7.808 | 45.454 | 1.00 | 19.84 | D |
| ATOM | 8090 | O | THR D | 576 | 131.878 | 7.565 | 44.358 | 1.00 | 20.54 | D |
| ATOM | 8091 | N | ILE D | 577 | 130.228 | 8.461 | 45.571 | 1.00 | 18.92 | D |
| ATOM | 8092 | CA | ILE D | 577 | 129.622 | 8.959 | 44.366 | 1.00 | 20.24 | D |
| ATOM | 8093 | CB | ILE D | 577 | 128.395 | 8.161 | 43.984 | 1.00 | 19.47 | D |
| ATOM | 8094 | CG2 | ILE D | 577 | 127.588 | 8.938 | 42.958 | 1.00 | 17.51 | D |
| ATOM | 8095 | CG1 | ILE D | 577 | 128.818 | 6.790 | 43.456 | 1.00 | 19.93 | D |
| ATOM | 8096 | CD1 | ILE D | 577 | 127.641 | 5.922 | 43.008 | 1.00 | 20.62 | D |
| ATOM | 8097 | C | ILE D | 577 | 129.232 | 10.403 | 44.528 | 1.00 | 21.53 | D |
| ATOM | 8098 | O | ILE D | 577 | 128.603 | 10.777 | 45.530 | 1.00 | 23.56 | D |
| ATOM | 8099 | N | TYR D | 578 | 129.615 | 11.223 | 43.551 | 1.00 | 20.82 | D |
| ATOM | 8100 | CA | TYR D | 578 | 129.264 | 12.644 | 43.593 | 1.00 | 21.04 | D |
| ATOM | 8101 | CB | TYR D | 578 | 130.384 | 13.509 | 42.982 | 1.00 | 21.65 | D |
| ATOM | 8102 | CG | TYR D | 578 | 130.022 | 14.975 | 42.792 | 1.00 | 22.52 | D |
| ATOM | 8103 | CD1 | TYR D | 578 | 129.168 | 15.625 | 43.690 | 1.00 | 22.16 | D |
| ATOM | 8104 | CE1 | TYR D | 578 | 128.795 | 16.960 | 43.506 | 1.00 | 21.60 | D |
| ATOM | 8105 | CD2 | TYR D | 578 | 130.510 | 15.702 | 41.701 | 1.00 | 21.12 | D |
| ATOM | 8106 | CE2 | TYR D | 578 | 130.147 | 17.038 | 41.506 | 1.00 | 21.36 | D |
| ATOM | 8107 | CZ | TYR D | 578 | 129.286 | 17.663 | 42.410 | 1.00 | 21.53 | D |
| ATOM | 8108 | OH | TYR D | 578 | 128.900 | 18.977 | 42.212 | 1.00 | 21.71 | D |
| ATOM | 8109 | C | TYR D | 578 | 127.955 | 12.883 | 42.851 | 1.00 | 20.91 | D |
| ATOM | 8110 | O | TYR D | 578 | 127.939 | 12.885 | 41.633 | 1.00 | 21.83 | D |
| ATOM | 8111 | N | ASP D | 579 | 126.866 | 13.094 | 43.596 | 1.00 | 22.68 | D |
| ATOM | 8112 | CA | ASP D | 579 | 125.534 | 13.342 | 43.017 | 1.00 | 24.76 | D |
| ATOM | 8113 | CB | ASP D | 579 | 124.440 | 13.037 | 44.054 | 1.00 | 25.04 | D |
| ATOM | 8114 | CG | ASP D | 579 | 123.029 | 12.930 | 43.443 | 1.00 | 27.17 | D |
| ATOM | 8115 | OD1 | ASP D | 579 | 122.790 | 13.506 | 42.363 | 1.00 | 26.43 | D |
| ATOM | 8116 | OD2 | ASP D | 579 | 122.144 | 12.271 | 44.061 | 1.00 | 27.38 | D |
| ATOM | 8117 | C | ASP D | 579 | 125.435 | 14.808 | 42.583 | 1.00 | 26.38 | D |
| ATOM | 8118 | O | ASP D | 579 | 124.715 | 15.590 | 43.200 | 1.00 | 25.82 | D |
| ATOM | 8119 | N | HIS D | 580 | 126.148 | 15.172 | 41.513 | 1.00 | 28.39 | D |
| ATOM | 8120 | CA | HIS D | 580 | 126.145 | 16.555 | 41.028 | 1.00 | 31.78 | D |
| ATOM | 8121 | CB | HIS D | 580 | 127.029 | 16.692 | 39.794 | 1.00 | 33.03 | D |
| ATOM | 8122 | CG | HIS D | 580 | 126.607 | 15.830 | 38.654 | 1.00 | 37.41 | D |
| ATOM | 8123 | CD2 | HIS D | 580 | 125.611 | 15.973 | 37.750 | 1.00 | 38.10 | D |
| ATOM | 8124 | ND1 | HIS D | 580 | 127.244 | 14.648 | 38.342 | 1.00 | 39.09 | D |
| ATOM | 8125 | CE1 | HIS D | 580 | 126.660 | 14.102 | 37.292 | 1.00 | 38.85 | D |
| ATOM | 8126 | NE2 | HIS D | 580 | 125.666 | 14.885 | 36.913 | 1.00 | 39.22 | D |
| ATOM | 8127 | C | HIS D | 580 | 124.745 | 17.113 | 40.723 | 1.00 | 33.01 | D |
| ATOM | 8128 | O | HIS D | 580 | 124.484 | 18.313 | 40.890 | 1.00 | 34.71 | D |
| ATOM | 8129 | N | GLU D | 581 | 123.836 | 16.251 | 40.292 | 1.00 | 33.24 | D |
| ATOM | 8130 | CA | GLU D | 581 | 122.486 | 16.693 | 39.995 | 1.00 | 32.71 | D |
| ATOM | 8131 | CB | GLU D | 581 | 121.700 | 15.544 | 39.375 | 1.00 | 35.03 | D |
| ATOM | 8132 | CG | GLU D | 581 | 120.463 | 15.978 | 38.652 | 1.00 | 39.07 | D |
| ATOM | 8133 | CD | GLU D | 581 | 119.702 | 14.818 | 38.092 | 1.00 | 41.55 | D |
| ATOM | 8134 | OE1 | GLU D | 581 | 120.332 | 14.026 | 37.350 | 1.00 | 43.80 | D |
| ATOM | 8135 | OE2 | GLU D | 581 | 118.485 | 14.712 | 38.394 | 1.00 | 41.52 | D |
| ATOM | 8136 | C | GLU D | 581 | 121.788 | 17.202 | 41.266 | 1.00 | 31.80 | D |
| ATOM | 8137 | O | GLU D | 581 | 121.244 | 18.306 | 41.260 | 1.00 | 31.97 | D |
| ATOM | 8138 | N | ARG D | 582 | 121.808 | 16.414 | 42.348 | 1.00 | 29.57 | D |
| ATOM | 8139 | CA | ARG D | 582 | 121.177 | 16.825 | 43.616 | 1.00 | 29.36 | D |
| ATOM | 8140 | CB | ARG D | 582 | 120.622 | 15.607 | 44.390 | 1.00 | 26.09 | D |
| ATOM | 8141 | CG | ARG D | 582 | 119.558 | 14.822 | 43.650 | 1.00 | 22.04 | D |
| ATOM | 8142 | CD | ARG D | 582 | 118.850 | 13.766 | 44.496 | 1.00 | 20.11 | D |
| ATOM | 8143 | NE | ARG D | 582 | 118.024 | 12.908 | 43.636 | 1.00 | 19.38 | D |
| ATOM | 8144 | CZ | ARG D | 582 | 118.506 | 12.137 | 42.658 | 1.00 | 20.74 | D |
| ATOM | 8145 | NH1 | ARG D | 582 | 119.808 | 12.105 | 42.415 | 1.00 | 22.11 | D |
| ATOM | 8146 | NH2 | ARG D | 582 | 117.695 | 11.398 | 41.908 | 1.00 | 19.51 | D |
| ATOM | 8147 | C | ARG D | 582 | 122.160 | 17.598 | 44.531 | 1.00 | 31.40 | D |
| ATOM | 8148 | O | ARG D | 582 | 121.796 | 18.063 | 45.622 | 1.00 | 29.69 | D |
| ATOM | 8149 | N | LYS D | 583 | 123.401 | 17.747 | 44.074 | 1.00 | 33.89 | D |
| ATOM | 8150 | CA | LYS D | 583 | 124.433 | 18.436 | 44.849 | 1.00 | 35.05 | D |
| ATOM | 8151 | CB | LYS D | 583 | 124.069 | 19.912 | 45.068 | 1.00 | 38.08 | D |
| ATOM | 8152 | CG | LYS D | 583 | 124.348 | 20.830 | 43.882 | 1.00 | 40.35 | D |
| ATOM | 8153 | CD | LYS D | 583 | 125.842 | 20.963 | 43.611 | 1.00 | 42.80 | D |
| ATOM | 8154 | CE | LYS D | 583 | 126.103 | 21.734 | 42.310 | 1.00 | 42.80 | D |
| ATOM | 8155 | NZ | LYS D | 583 | 125.618 | 23.153 | 42.366 | 1.00 | 43.25 | D |
| ATOM | 8156 | C | LYS D | 583 | 124.627 | 17.755 | 46.202 | 1.00 | 34.05 | D |
| ATOM | 8157 | O | LYS D | 583 | 124.524 | 18.401 | 47.241 | 1.00 | 35.02 | D |
| ATOM | 8158 | N | ARG D | 584 | 124.898 | 16.449 | 46.172 | 1.00 | 33.23 | D |
| ATOM | 8159 | CA | ARG D | 584 | 125.128 | 15.634 | 47.370 | 1.00 | 31.46 | D |
| ATOM | 8160 | CB | ARG D | 584 | 123.854 | 14.861 | 47.768 | 1.00 | 29.44 | D |
| ATOM | 8161 | CG | ARG D | 584 | 122.595 | 15.702 | 47.938 | 1.00 | 30.45 | D |
| ATOM | 8162 | CD | ARG D | 584 | 121.369 | 14.815 | 48.175 | 1.00 | 30.86 | D |
| ATOM | 8163 | NE | ARG D | 584 | 120.098 | 15.542 | 48.113 | 1.00 | 31.46 | D |
| ATOM | 8164 | CZ | ARG D | 584 | 118.904 | 14.965 | 48.229 | 1.00 | 32.25 | D |
| ATOM | 8165 | NH1 | ARG D | 584 | 118.820 | 13.653 | 48.414 | 1.00 | 29.52 | D |
| ATOM | 8166 | NH2 | ARG D | 584 | 117.793 | 15.697 | 48.156 | 1.00 | 30.64 | D |
| ATOM | 8167 | C | ARG D | 584 | 126.247 | 14.622 | 47.065 | 1.00 | 31.14 | D |
| ATOM | 8168 | O | ARG D | 584 | 126.668 | 14.466 | 45.916 | 1.00 | 30.87 | D |
| ATOM | 8169 | N | CYS D | 585 | 126.732 | 13.961 | 48.111 | 1.00 | 30.50 | D |
| ATOM | 8170 | CA | CYS D | 585 | 127.762 | 12.937 | 48.009 | 1.00 | 29.49 | D |
| ATOM | 8171 | CB | CYS D | 585 | 129.086 | 13.427 | 48.598 | 1.00 | 32.51 | D |
| ATOM | 8172 | SG | CYS D | 585 | 130.268 | 12.081 | 49.098 | 1.00 | 36.50 | D |
| ATOM | 8173 | C | CYS D | 585 | 127.298 | 11.719 | 48.809 | 1.00 | 28.24 | D |
| ATOM | 8174 | O | CYS D | 585 | 126.814 | 11.850 | 49.943 | 1.00 | 28.12 | D |
| ATOM | 8175 | N | ILE D | 586 | 127.434 | 10.546 | 48.193 | 1.00 | 25.25 | D |
| ATOM | 8176 | CA | ILE D | 586 | 127.094 | 9.266 | 48.806 | 1.00 | 22.27 | D |
| ATOM | 8177 | CB | ILE D | 586 | 126.394 | 8.304 | 47.804 | 1.00 | 21.75 | D |
| ATOM | 8178 | CG2 | ILE D | 586 | 126.164 | 6.967 | 48.464 | 1.00 | 18.50 | D |
| ATOM | 8179 | CG1 | ILE D | 586 | 125.070 | 8.900 | 47.279 | 1.00 | 21.75 | D |
| ATOM | 8180 | CD1 | ILE D | 586 | 125.217 | 9.902 | 46.122 | 1.00 | 19.83 | D |
| ATOM | 8181 | C | ILE D | 586 | 128.460 | 8.670 | 49.123 | 1.00 | 22.41 | D |
| ATOM | 8182 | O | ILE D | 586 | 129.400 | 8.875 | 48.359 | 1.00 | 24.24 | D |
| ATOM | 8183 | N | SER D | 587 | 128.586 | 7.955 | 50.236 | 1.00 | 21.77 | D |
| ATOM | 8184 | CA | SER D | 587 | 129.856 | 7.319 | 50.595 | 1.00 | 19.65 | D |
| ATOM | 8185 | CB | SER D | 587 | 130.644 | 8.217 | 51.548 | 1.00 | 18.81 | D |
| ATOM | 8186 | OC | SER D | 587 | 129.942 | 8.436 | 52.758 | 1.00 | 17.09 | D |
| ATOM | 8187 | C | SER D | 587 | 129.579 | 5.968 | 51.255 | 1.00 | 19.73 | D |
| ATOM | 8188 | O | SER D | 587 | 128.676 | 5.867 | 52.082 | 1.00 | 20.77 | D |
| ATOM | 8189 | N | ALA D | 588 | 130.334 | 4.936 | 50.874 | 1.00 | 19.14 | D |
| ATOM | 8190 | CA | ALA D | 588 | 130.169 | 3.595 | 51.436 | 1.00 | 18.83 | D |
| ATOM | 8191 | CB | ALA D | 588 | 129.817 | 2.586 | 50.342 | 1.00 | 17.57 | D |
| ATOM | 8192 | C | ALA D | 588 | 131.483 | 3.220 | 52.105 | 1.00 | 18.48 | D |
| ATOM | 8193 | O | ALA D | 588 | 132.549 | 3.280 | 51.482 | 1.00 | 18.12 | D |
| ATOM | 8194 | N | SER D | 589 | 131.387 | 2.829 | 53.375 | 1.00 | 19.29 | D |
| ATOM | 8195 | CA | SER D | 589 | 132.546 | 2.500 | 54.224 | 1.00 | 20.33 | D |
| ATOM | 8196 | CB | SER D | 589 | 132.552 | 3.487 | 55.416 | 1.00 | 21.31 | D |
| ATOM | 8197 | OG | SER D | 589 | 133.427 | 3.106 | 56.474 | 1.00 | 19.54 | D |
| ATOM | 8198 | C | SER D | 589 | 132.633 | 1.058 | 54.749 | 1.00 | 20.03 | D |
| ATOM | 8199 | O | SER D | 589 | 131.638 | 0.322 | 54.743 | 1.00 | 20.59 | D |
| ATOM | 8200 | N | MSE D | 590 | 133.837 | 0.677 | 55.189 | 1.00 | 22.56 | D |
| ATOM | 8201 | CA | MSE D | 590 | 134.089 | −0.644 | 55.760 | 1.00 | 25.48 | D |
| ATOM | 8202 | CB | MSE D | 590 | 135.578 | −0.827 | 56.067 | 1.00 | 28.07 | D |
| ATOM | 8203 | CG | MSE D | 590 | 135.932 | −2.225 | 56.531 | 1.00 | 30.40 | D |
| ATOM | 8204 | SE | MSE D | 590 | 137.843 | −2.501 | 56.672 | 1.00 | 39.74 | D |
| ATOM | 8205 | CE | MSE D | 590 | 138.123 | −3.610 | 55.120 | 1.00 | 33.01 | D |
| ATOM | 8206 | C | MSE D | 590 | 133.271 | −0.777 | 57.053 | 1.00 | 26.26 | D |
| ATOM | 8207 | O | MSE D | 590 | 133.045 | −1.883 | 57.540 | 1.00 | 27.96 | D |
| ATOM | 8208 | N | ASP D | 591 | 132.806 | 0.352 | 57.587 | 1.00 | 24.74 | D |
| ATOM | 8209 | CA | ASP D | 591 | 131.999 | 0.333 | 58.790 | 1.00 | 24.47 | D |
| ATOM | 8210 | CB | ASP D | 591 | 131.931 | 1.722 | 59.447 | 1.00 | 25.09 | D |
| ATOM | 8211 | CG | ASP D | 591 | 131.115 | 2.730 | 58.635 | 1.00 | 26.02 | D |
| ATOM | 8212 | OD1 | ASP D | 591 | 130.116 | 2.338 | 57.987 | 1.00 | 23.16 | D |
| ATOM | 8213 | OD2 | ASP D | 591 | 131.461 | 3.930 | 58.668 | 1.00 | 28.61 | D |
| ATOM | 8214 | C | ASP D | 591 | 130.575 | −0.167 | 58.535 | 1.00 | 25.63 | D |
| ATOM | 8215 | O | ASP D | 591 | 129.731 | −0.111 | 59.435 | 1.00 | 27.48 | D |
| ATOM | 8216 | N | THR D | 592 | 130.280 | −0.612 | 57.315 | 1.00 | 24.13 | D |
| ATOM | 8217 | CA | THR D | 592 | 128.953 | −1.144 | 57.027 | 1.00 | 23.60 | D |
| ATOM | 8218 | CB | THR D | 592 | 128.479 | −2.010 | 58.217 | 1.00 | 23.93 | D |
| ATOM | 8219 | OG1 | THR D | 592 | 129.276 | −3.197 | 58.290 | 1.00 | 24.58 | D |
| ATOM | 8220 | CG2 | THR D | 592 | 127.032 | −2.388 | 58.071 | 1.00 | 25.94 | D |
| ATOM | 8221 | C | THR D | 592 | 127.858 | −0.117 | 56.694 | 1.00 | 23.10 | D |
| ATOM | 8222 | O | THR D | 592 | 126.814 | −0.466 | 56.131 | 1.00 | 23.01 | D |
| ATOM | 8223 | N | THR D | 593 | 128.086 | 1.146 | 57.020 | 1.00 | 21.17 | D |
| ATOM | 8224 | CA | THR D | 593 | 127.084 | 2.164 | 56.743 | 1.00 | 20.07 | D |
| ATOM | 8225 | CB | THR D | 593 | 127.097 | 3.238 | 57.829 | 1.00 | 19.96 | D |
| ATOM | 8226 | OG1 | THR D | 593 | 128.317 | 3.981 | 57.736 | 1.00 | 20.19 | D |
| ATOM | 8227 | CG2 | THR D | 593 | 127.012 | 2.601 | 59.205 | 1.00 | 20.49 | D |
| ATOM | 8228 | C | THR D | 593 | 127.303 | 2.861 | 55.400 | 1.00 | 20.54 | D |
| ATOM | 8229 | O | THR D | 593 | 128.282 | 2.602 | 54.705 | 1.00 | 20.50 | D |
| ATOM | 8230 | N | ILE D | 594 | 126.360 | 3.728 | 55.039 | 1.00 | 19.85 | D |
| ATOM | 8231 | CA | ILE D | 594 | 126.422 | 4.520 | 53.823 | 1.00 | 18.48 | D |
| ATOM | 8232 | CB | ILE D | 594 | 125.423 | 4.012 | 52.749 | 1.00 | 17.96 | D |
| ATOM | 8233 | CG2 | ILE D | 594 | 125.476 | 4.931 | 51.512 | 1.00 | 16.27 | D |
| ATOM | 8234 | CG1 | ILE D | 594 | 125.763 | 2.564 | 52.352 | 1.00 | 17.94 | D |
| ATOM | 8235 | CD1 | ILE D | 594 | 124.874 | 1.976 | 51.244 | 1.00 | 13.75 | D |
| ATOM | 8236 | C | ILE D | 594 | 126.013 | 5.920 | 54.297 | 1.00 | 20.05 | D |
| ATOM | 8237 | O | ILE D | 594 | 125.125 | 6.060 | 55.147 | 1.00 | 19.44 | D |
| ATOM | 8238 | N | ARG D | 595 | 126.661 | 6.961 | 53.787 | 1.00 | 21.73 | D |
| ATOM | 8239 | CA | ARG D | 595 | 126.294 | 8.304 | 54.229 | 1.00 | 24.25 | D |
| ATOM | 8240 | CE | ARG D | 595 | 127.380 | 8.898 | 55.133 | 1.00 | 26.38 | D |
| ATOM | 8241 | CG | ARG D | 595 | 127.485 | 8.261 | 56.508 | 1.00 | 27.19 | D |
| ATOM | 8242 | CD | ARG D | 595 | 128.489 | 9.003 | 57.373 | 1.00 | 29.27 | D |
| ATOM | 8243 | NE | ARG D | 595 | 128.184 | 8.848 | 58.797 | 1.00 | 33.82 | D |
| ATOM | 8244 | CZ | ARG D | 595 | 128.465 | 9.753 | 59.732 | 1.00 | 34.00 | D |
| ATOM | 8245 | NH1 | ARG D | 595 | 129.068 | 10.889 | 59.398 | 1.00 | 34.40 | D |
| ATOM | 8246 | NH2 | ARG D | 595 | 128.125 | 9.528 | 60.998 | 1.00 | 33.02 | D |
| ATOM | 8247 | C | ARG D | 595 | 125.999 | 9.282 | 53.108 | 1.00 | 24.90 | D |
| ATOM | 8248 | O | ARG D | 595 | 126.702 | 9.329 | 52.102 | 1.00 | 25.43 | D |
| ATOM | 8249 | N | ILE D | 596 | 124.939 | 10.056 | 53.294 | 1.00 | 25.81 | D |
| ATOM | 8250 | CA | ILE D | 596 | 124.540 | 11.056 | 52.319 | 1.00 | 27.39 | D |
| ATOM | 8251 | CB | ILE D | 596 | 123.000 | 11.093 | 52.138 | 1.00 | 27.40 | D |
| ATOM | 8252 | CG2 | ILE D | 596 | 122.646 | 11.950 | 50.935 | 1.00 | 27.77 | D |
| ATOM | 8253 | CG1 | ILE D | 596 | 122.459 | 9.681 | 51.891 | 1.00 | 27.13 | D |
| ATOM | 8254 | CD1 | ILE D | 596 | 122.953 | 9.062 | 50.590 | 1.00 | 26.64 | D |
| ATOM | 8255 | C | ILE D | 596 | 125.013 | 12.392 | 52.870 | 1.00 | 28.16 | D |
| ATOM | 8256 | O | ILE D | 596 | 124.637 | 12.787 | 53.976 | 1.00 | 28.61 | D |
| ATOM | 8257 | N | TRP D | 597 | 125.862 | 13.078 | 52.118 | 1.00 | 28.67 | D |
| ATOM | 8258 | CA | TRP D | 597 | 126.368 | 14.368 | 52.568 | 1.00 | 28.04 | D |
| ATOM | 8259 | CE | TRP D | 597 | 127.893 | 14.391 | 52.515 | 1.00 | 29.61 | D |
| ATOM | 8260 | CG | TRP D | 597 | 128.562 | 13.228 | 53.155 | 1.00 | 28.57 | D |
| ATOM | 8261 | CD2 | TRP D | 597 | 129.116 | 13.186 | 54.470 | 1.00 | 27.43 | D |
| ATOM | 8262 | CE2 | TRP D | 597 | 129.707 | 11.918 | 54.634 | 1.00 | 27.37 | D |
| ATOM | 8263 | CE3 | TRP D | 597 | 129.172 | 14.103 | 55.529 | 1.00 | 27.46 | D |
| ATOM | 8264 | CD1 | TRP D | 597 | 128.822 | 12.016 | 52.589 | 1.00 | 29.00 | D |
| ATOM | 8265 | NE1 | TRP D | 597 | 129.516 | 11.221 | 53.469 | 1.00 | 27.32 | D |
| ATOM | 8266 | CZ2 | TRP D | 597 | 130.349 | 11.541 | 55.817 | 1.00 | 27.76 | D |
| ATOM | 8267 | CZ3 | TRP D | 597 | 129.810 | 13.729 | 56.703 | 1.00 | 27.02 | D |
| ATOM | 8268 | CR2 | TRP D | 597 | 130.389 | 12.457 | 56.838 | 1.00 | 27.58 | D |
| ATOM | 8269 | C | TRP D | 597 | 125.838 | 15.486 | 51.684 | 1.00 | 27.75 | D |
| ATOM | 8270 | O | TRP D | 597 | 125.729 | 15.326 | 50.472 | 1.00 | 27.73 | D |
| ATOM | 8271 | N | ASP D | 598 | 125.519 | 16.624 | 52.284 | 1.00 | 28.69 | D |
| ATOM | 8272 | CA | ASP D | 598 | 125.012 | 17.773 | 51.527 | 1.00 | 29.82 | D |
| ATOM | 8273 | CB | ASP D | 598 | 123.966 | 18.511 | 52.381 | 1.00 | 29.96 | D |
| ATOM | 8274 | CG | ASP D | 598 | 123.483 | 19.816 | 51.749 | 1.00 | 30.01 | D |
| ATOM | 8275 | OD1 | ASP D | 598 | 123.889 | 20.118 | 50.601 | 1.00 | 29.40 | D |
| ATOM | 8276 | OD2 | ASP D | 598 | 122.700 | 20.537 | 52.417 | 1.00 | 28.15 | D |
| ATOM | 8277 | C | ASP D | 598 | 126.197 | 18.694 | 51.177 | 1.00 | 28.75 | D |
| ATOM | 8278 | O | ASP D | 598 | 126.880 | 19.192 | 52.067 | 1.00 | 26.58 | D |
| ATOM | 8279 | N | LEU D | 599 | 126.442 | 18.912 | 49.884 | 1.00 | 29.62 | D |
| ATOM | 8280 | CA | LEU D | 599 | 127.567 | 19.753 | 49.466 | 1.00 | 32.72 | D |
| ATOM | 8281 | CB | LEU D | 599 | 128.148 | 19.244 | 48.141 | 1.00 | 30.82 | D |
| ATOM | 8282 | CG | LEU D | 599 | 128.646 | 17.780 | 48.141 | 1.00 | 31.03 | D |
| ATOM | 8283 | CD1 | LEU D | 599 | 129.360 | 17.472 | 46.822 | 1.00 | 29.75 | D |
| ATOM | 8284 | CD2 | LEU D | 599 | 129.579 | 17.529 | 49.309 | 1.00 | 28.77 | D |
| ATOM | 8285 | C | LEU D | 599 | 127.238 | 21.246 | 49.383 | 1.00 | 35.04 | D |
| ATOM | 8286 | O | LEU D | 599 | 127.899 | 22.009 | 48.686 | 1.00 | 34.05 | D |
| ATOM | 8287 | N | GLU D | 600 | 126.228 | 21.651 | 50.145 | 1.00 | 39.89 | D |
| ATOM | 8288 | CA | GLU D | 600 | 125.793 | 23.041 | 50.205 | 1.00 | 42.71 | D |
| ATOM | 8289 | CB | GLU D | 600 | 124.279 | 23.118 | 49.998 | 1.00 | 43.83 | D |
| ATOM | 8290 | CG | GLU D | 600 | 123.818 | 22.578 | 48.659 | 1.00 | 49.15 | D |
| ATOM | 8291 | CD | GLU D | 600 | 124.057 | 23.565 | 47.524 | 1.00 | 52.47 | D |
| ATOM | 8292 | OE1 | GLU D | 600 | 123.836 | 23.205 | 46.340 | 1.00 | 54.37 | D |
| ATOM | 8293 | OE2 | GLU D | 600 | 124.462 | 24.710 | 47.820 | 1.00 | 53.81 | D |
| ATOM | 8294 | C | GLU D | 600 | 126.154 | 23.614 | 51.581 | 1.00 | 43.08 | D |
| ATOM | 8295 | O | GLU D | 600 | 125.956 | 24.799 | 51.847 | 1.00 | 44.92 | D |
| ATOM | 8296 | N | ASN D | 605 | 126.681 | 22.769 | 52.461 | 1.00 | 42.60 | D |
| ATOM | 8297 | CA | ASN D | 605 | 127.049 | 23.217 | 53.798 | 1.00 | 41.25 | D |
| ATOM | 8298 | CB | ASN D | 605 | 125.787 | 23.392 | 54.640 | 1.00 | 41.21 | D |
| ATOM | 8299 | CG | ASN D | 605 | 125.003 | 22.105 | 54.794 | 1.00 | 42.82 | D |
| ATOM | 8300 | OD1 | ASN D | 605 | 124.753 | 21.383 | 53.830 | 1.00 | 41.44 | D |
| ATOM | 8301 | ND2 | ASN D | 605 | 124.599 | 21.819 | 56.024 | 1.00 | 44.97 | D |
| ATOM | 8302 | C | ASN D | 605 | 128.006 | 22.222 | 54.440 | 1.00 | 40.16 | D |
| ATOM | 8303 | O | ASN D | 605 | 128.507 | 22.442 | 55.544 | 1.00 | 39.89 | D |
| ATOM | 8304 | N | GLY D | 606 | 128.259 | 21.135 | 53.712 | 1.00 | 39.38 | D |
| ATOM | 8305 | CA | GLY D | 606 | 129.165 | 20.091 | 54.156 | 1.00 | 36.83 | D |
| ATOM | 8306 | C | GLY D | 606 | 128.629 | 19.279 | 55.311 | 1.00 | 35.89 | D |
| ATOM | 8307 | O | GLY D | 606 | 129.336 | 18.446 | 55.865 | 1.00 | 35.05 | D |
| ATOM | 8308 | N | GLU D | 607 | 127.378 | 19.523 | 55.675 | 1.00 | 35.04 | D |
| ATOM | 8309 | CA | GLU D | 607 | 126.771 | 18.814 | 56.779 | 1.00 | 35.34 | D |
| ATOM | 8310 | CB | GLU D | 607 | 125.624 | 19.635 | 57.350 | 1.00 | 38.59 | D |
| ATOM | 8311 | CG | GLU D | 607 | 126.032 | 20.652 | 58.401 | 1.00 | 44.13 | D |
| ATOM | 8312 | CD | GLU D | 607 | 124.828 | 21.150 | 59.183 | 1.00 | 49.69 | D |
| ATOM | 8313 | OE1 | GLU D | 607 | 124.093 | 20.289 | 59.749 | 1.00 | 51.01 | D |
| ATOM | 8314 | OE2 | GLU D | 607 | 124.608 | 22.394 | 59.227 | 1.00 | 52.33 | D |
| ATOM | 8315 | C | GLU D | 607 | 126.271 | 17.415 | 56.411 | 1.00 | 33.71 | D |
| ATOM | 8316 | O | GLU D | 607 | 125.991 | 17.144 | 55.247 | 1.00 | 33.25 | D |
| ATOM | 8317 | N | LEU D | 608 | 126.175 | 16.521 | 57.397 | 1.00 | 31.85 | D |
| ATOM | 8318 | CA | LEU D | 608 | 125.686 | 15.175 | 57.125 | 1.00 | 30.51 | D |
| ATOM | 8319 | CE | LEU D | 608 | 126.052 | 14.216 | 58.253 | 1.00 | 27.96 | D |
| ATOM | 8320 | CG | LEU D | 608 | 125.557 | 12.778 | 58.041 | 1.00 | 28.56 | D |
| ATOM | 8321 | CD1 | LEU D | 608 | 126.456 | 12.051 | 57.046 | 1.00 | 24.69 | D |
| ATOM | 8322 | CD2 | LEU D | 608 | 125.531 | 12.032 | 59.394 | 1.00 | 28.82 | D |
| ATOM | 8323 | C | LEU D | 608 | 124.169 | 15.253 | 56.971 | 1.00 | 30.57 | D |
| ATOM | 8324 | O | LEU D | 608 | 123.532 | 16.124 | 57.573 | 1.00 | 31.18 | D |
| ATOM | 8325 | N | MEE D | 625 | 123.605 | 14.363 | 56.150 | 1.00 | 29.87 | D |
| ATOM | 8326 | CA | MSE D | 625 | 122.153 | 14.303 | 55.888 | 1.00 | 30.31 | D |
| ATOM | 8327 | CB | MSE D | 625 | 121.846 | 14.367 | 54.381 | 1.00 | 31.08 | D |
| ATOM | 8328 | CG | MSE D | 625 | 122.016 | 15.737 | 53.754 | 1.00 | 33.02 | D |
| ATOM | 8329 | SE | MSE D | 625 | 121.598 | 15.802 | 51.862 | 1.00 | 38.72 | D |
| ATOM | 8330 | CE | MSE D | 625 | 119.705 | 15.354 | 51.962 | 1.00 | 32.55 | D |
| ATOM | 8331 | C | MSE D | 625 | 121.523 | 13.022 | 56.410 | 1.00 | 27.88 | D |
| ATOM | 8332 | O | MSE D | 625 | 120.538 | 13.032 | 57.145 | 1.00 | 28.06 | D |
| ATOM | 8333 | N | TYR D | 626 | 122.073 | 11.901 | 55.993 | 1.00 | 25.99 | D |
| ATOM | 8334 | CA | TYR D | 626 | 121.505 | 10.670 | 56.444 | 1.00 | 24.99 | D |
| ATOM | 8335 | CB | TYR D | 626 | 120.453 | 10.176 | 55.461 | 1.00 | 25.25 | D |
| ATOM | 8336 | CG | TYR D | 626 | 119.434 | 11.186 | 54.999 | 1.00 | 24.62 | D |
| ATOM | 8337 | CD1 | TYR D | 626 | 118.409 | 11.612 | 55.842 | 1.00 | 21.86 | D |
| ATOM | 8338 | CE1 | TYR D | 626 | 117.391 | 12.424 | 55.363 | 1.00 | 23.49 | D |
| ATOM | 8339 | CD2 | TYR D | 626 | 119.426 | 11.615 | 53.669 | 1.00 | 24.69 | D |
| ATOM | 8340 | CE2 | TYR D | 626 | 118.421 | 12.421 | 53.179 | 1.00 | 24.51 | D |
| ATOM | 8341 | CZ | TYR D | 626 | 117.396 | 12.820 | 54.023 | 1.00 | 24.72 | D |
| ATOM | 8342 | OH | TYR D | 626 | 116.346 | 13.555 | 53.502 | 1.00 | 25.76 | D |
| ATOM | 8343 | C | TYR D | 626 | 122.555 | 9.597 | 56.565 | 1.00 | 23.88 | D |
| ATOM | 8344 | O | TYR D | 626 | 123.605 | 9.652 | 55.923 | 1.00 | 23.89 | D |
| ATOM | 8345 | N | THR D | 627 | 122.255 | 8.616 | 57.406 | 1.00 | 23.42 | D |
| ATOM | 8346 | CA | THR D | 627 | 123.122 | 7.472 | 57.564 | 1.00 | 20.62 | D |
| ATOM | 8347 | CE | THR D | 627 | 123.589 | 7.271 | 58.987 | 1.00 | 19.43 | D |
| ATOM | 8348 | OG1 | THR D | 627 | 124.268 | 8.447 | 59.440 | 1.00 | 20.14 | D |
| ATOM | 8349 | CG2 | THR D | 627 | 124.535 | 6.066 | 59.050 | 1.00 | 17.31 | D |
| ATOM | 8350 | C | THR D | 627 | 122.235 | 6.308 | 57.191 | 1.00 | 19.41 | D |
| ATOM | 8351 | O | THR D | 627 | 121.244 | 6.050 | 57.859 | 1.00 | 18.63 | D |
| ATOM | 8352 | N | LEU D | 628 | 122.577 | 5.640 | 56.095 | 1.00 | 19.47 | D |
| ATOM | 8353 | CA | LEU D | 628 | 121.821 | 4.485 | 55.626 | 1.00 | 19.11 | D |
| ATOM | 8354 | CE | LEU D | 628 | 121.755 | 4.469 | 54.108 | 1.00 | 17.80 | D |
| ATOM | 8355 | CG | LEU D | 628 | 121.117 | 5.669 | 53.396 | 1.00 | 16.59 | D |
| ATOM | 8356 | CD1 | LEU D | 628 | 121.092 | 5.364 | 51.890 | 1.00 | 16.34 | D |
| ATOM | 8357 | CD2 | LEU D | 628 | 119.712 | 5.929 | 53.916 | 1.00 | 13.86 | D |
| ATOM | 8358 | C | LEU D | 628 | 122.523 | 3.227 | 56.111 | 1.00 | 19.57 | D |
| ATOM | 8359 | O | LEU D | 628 | 123.674 | 2.966 | 55.744 | 1.00 | 20.61 | D |
| ATOM | 8360 | N | GLN D | 629 | 121.817 | 2.456 | 56.936 | 1.00 | 19.21 | D |
| ATOM | 8361 | CA | GLN D | 629 | 122.331 | 1.216 | 57.512 | 1.00 | 19.59 | D |
| ATOM | 8362 | CE | GLN D | 629 | 122.142 | 1.262 | 59.031 | 1.00 | 18.99 | D |
| ATOM | 8363 | CG | GLN D | 629 | 122.640 | 0.047 | 59.795 | 1.00 | 19.95 | D |
| ATOM | 8364 | CD | GLN D | 629 | 124.155 | −0.088 | 59.756 | 1.00 | 21.73 | D |
| ATOM | 8365 | OE1 | GLN D | 629 | 124.709 | −0.697 | 58.841 | 1.00 | 21.66 | D |
| ATOM | 8366 | NE2 | GLN D | 629 | 124.834 | 0.492 | 60.751 | 1.00 | 21.48 | D |
| ATOM | 8367 | C | GLN D | 629 | 121.565 | 0.034 | 56.922 | 1.00 | 19.26 | D |
| ATOM | 8368 | O | GLN D | 629 | 120.435 | −0.224 | 57.313 | 1.00 | 22.24 | D |
| ATOM | 8369 | N | GLY D | 630 | 122.178 | −0.689 | 55.993 | 1.00 | 18.64 | D |
| ATOM | 8370 | CA | GLY D | 630 | 121.480 | −1.802 | 55.386 | 1.00 | 19.70 | D |
| ATOM | 8371 | C | GLY D | 630 | 122.361 | −2.917 | 54.860 | 1.00 | 21.44 | D |
| ATOM | 8372 | O | GLY D | 630 | 122.068 | −3.533 | 53.844 | 1.00 | 22.81 | D |
| ATOM | 8373 | N | HIS D | 631 | 123.459 | −3.179 | 55.544 | 1.00 | 22.39 | D |
| ATOM | 8374 | CA | HIS D | 631 | 124.368 | −4.236 | 55.143 | 1.00 | 23.12 | D |
| ATOM | 8375 | CB | HIS D | 631 | 125.425 | −3.720 | 54.169 | 1.00 | 26.25 | D |
| ATOM | 8376 | CG | HIS D | 631 | 124.856 | −3.144 | 52.915 | 1.00 | 28.65 | D |
| ATOM | 8377 | CD2 | HIS D | 631 | 124.544 | −1.870 | 52.586 | 1.00 | 28.78 | D |
| ATOM | 8378 | ND1 | HIS D | 631 | 124.504 | −3.920 | 51.833 | 1.00 | 29.43 | D |
| ATOM | 8379 | CE1 | HIS D | 631 | 124.002 | −3.146 | 50.887 | 1.00 | 28.34 | D |
| ATOM | 8380 | NE2 | HIS D | 631 | 124.015 | −1.898 | 51.319 | 1.00 | 28.49 | D |
| ATOM | 8381 | C | HIS D | 631 | 125.030 | −4.618 | 56.440 | 1.00 | 22.81 | D |
| ATOM | 8382 | O | HIS D | 631 | 125.008 | −3.860 | 57.402 | 1.00 | 25.35 | D |
| ATOM | 8383 | N | THR D | 632 | 125.661 | −5.774 | 56.455 | 1.00 | 21.41 | D |
| ATOM | 8384 | CA | THR D | 632 | 126.288 | −6.261 | 57.663 | 1.00 | 20.28 | D |
| ATOM | 8385 | CB | THR D | 632 | 125.646 | −7.591 | 58.047 | 1.00 | 18.49 | D |
| ATOM | 8386 | OG1 | THR D | 632 | 125.767 | −8.524 | 56.957 | 1.00 | 18.30 | D |
| ATOM | 8387 | CG2 | THR D | 632 | 124.173 | −7.371 | 58.349 | 1.00 | 13.99 | D |
| ATOM | 8388 | C | THR D | 632 | 127.771 | −6.466 | 57.509 | 1.00 | 20.33 | D |
| ATOM | 8389 | O | THR D | 632 | 128.427 | −6.983 | 58.405 | 1.00 | 22.76 | D |
| ATOM | 8390 | N | ALA D | 633 | 128.291 | −6.053 | 56.367 | 1.00 | 19.78 | D |
| ATOM | 8391 | CA | ALA D | 633 | 129.695 | −6.224 | 56.061 | 1.00 | 18.83 | D |
| ATOM | 8392 | CB | ALA D | 633 | 129.877 | −7.469 | 55.239 | 1.00 | 17.26 | D |
| ATOM | 8393 | C | ALA D | 633 | 130.169 | −5.029 | 55.282 | 1.00 | 19.55 | D |
| ATOM | 8394 | O | ALA D | 633 | 129.385 | −4.126 | 54.991 | 1.00 | 19.68 | D |
| ATOM | 8395 | N | LEU D | 634 | 131.455 | −5.024 | 54.951 | 1.00 | 20.72 | D |
| ATOM | 8396 | CA | LEU D | 634 | 132.051 | −3.944 | 54.175 | 1.00 | 20.81 | D |
| ATOM | 8397 | CB | LEU D | 634 | 133.478 | −4.347 | 53.790 | 1.00 | 21.47 | D |
| ATOM | 8398 | CG | LEU D | 634 | 134.286 | −3.752 | 52.639 | 1.00 | 22.59 | D |
| ATOM | 8399 | CD1 | LEU D | 634 | 133.982 | −4.534 | 51.395 | 1.00 | 25.22 | D |
| ATOM | 8400 | CD2 | LEU D | 634 | 133.968 | −2.263 | 52.448 | 1.00 | 26.73 | D |
| ATOM | 8401 | C | LEU D | 634 | 131.211 | −3.634 | 52.940 | 1.00 | 21.70 | D |
| ATOM | 8402 | O | LEU D | 634 | 130.889 | −4.523 | 52.138 | 1.00 | 22.37 | D |
| ATOM | 8403 | N | VAL D | 635 | 130.822 | −2.371 | 52.807 | 1.00 | 21.89 | D |
| ATOM | 8404 | CA | VAL D | 635 | 130.038 | −1.957 | 51.646 | 1.00 | 23.38 | D |
| ATOM | 8405 | CB | VAL D | 635 | 128.929 | −0.951 | 52.019 | 1.00 | 23.26 | D |
| ATOM | 8406 | CG1 | VAL D | 635 | 128.136 | −0.574 | 50.771 | 1.00 | 25.04 | D |
| ATOM | 8407 | CG2 | VAL D | 635 | 128.008 | −1.545 | 53.048 | 1.00 | 21.55 | D |
| ATOM | 8408 | C | VAL D | 635 | 131.019 | −1.303 | 50.670 | 1.00 | 24.61 | D |
| ATOM | 8409 | O | VAL D | 635 | 131.455 | −0.164 | 50.878 | 1.00 | 22.67 | D |
| ATOM | 8410 | N | GLY D | 636 | 131.352 | −2.036 | 49.607 | 1.00 | 25.64 | D |
| ATOM | 8411 | CA | GLY D | 636 | 132.320 | −1.552 | 48.641 | 1.00 | 26.53 | D |
| ATOM | 8412 | C | GLY D | 636 | 131.874 | −1.151 | 47.254 | 1.00 | 26.66 | D |
| ATOM | 8413 | O | GLY D | 636 | 132.687 | −0.595 | 46.509 | 1.00 | 27.11 | D |
| ATOM | 8414 | N | LEU D | 637 | 130.612 | −1.418 | 46.906 | 1.00 | 26.43 | D |
| ATOM | 8415 | CA | LEU D | 637 | 130.077 | −1.089 | 45.582 | 1.00 | 24.26 | D |
| ATOM | 8416 | CB | LEU D | 637 | 129.705 | −2.367 | 44.825 | 1.00 | 22.49 | D |
| ATOM | 8417 | CG | LEU D | 637 | 130.896 | −3.277 | 44.542 | 1.00 | 21.34 | D |
| ATOM | 8418 | CD1 | LEU D | 637 | 132.028 | −2.399 | 44.003 | 1.00 | 18.59 | D |
| ATOM | 8419 | CD2 | LEU D | 637 | 131.352 | −3.982 | 45.809 | 1.00 | 23.19 | D |
| ATOM | 8420 | C | LEU D | 637 | 128.868 | −0.163 | 45.606 | 1.00 | 25.18 | D |
| ATOM | 8421 | O | LEU D | 637 | 127.843 | −0.503 | 46.182 | 1.00 | 26.31 | D |
| ATOM | 8422 | N | LEU D | 638 | 129.009 | 1.004 | 44.970 | 1.00 | 26.27 | D |
| ATOM | 8423 | CA | LEU D | 638 | 127.959 | 2.021 | 44.854 | 1.00 | 25.24 | D |
| ATOM | 8424 | CB | LEU D | 638 | 128.335 | 3.308 | 45.579 | 1.00 | 25.17 | D |
| ATOM | 8425 | CG | LEU D | 638 | 128.217 | 3.292 | 47.092 | 1.00 | 26.38 | D |
| ATOM | 8426 | CD1 | LEU D | 638 | 128.445 | 4.708 | 47.617 | 1.00 | 25.29 | D |
| ATOM | 8427 | CD2 | LEU D | 638 | 126.827 | 2.778 | 47.484 | 1.00 | 23.90 | D |
| ATOM | 8428 | C | LEU D | 638 | 127.772 | 2.372 | 43.396 | 1.00 | 25.47 | D |
| ATOM | 8429 | O | LEU D | 638 | 128.746 | 2.427 | 42.638 | 1.00 | 24.74 | D |
| ATOM | 8430 | N | ARG D | 639 | 126.515 | 2.639 | 43.029 | 1.00 | 25.82 | D |
| ATOM | 8431 | CA | ARG D | 639 | 126.101 | 2.999 | 41.661 | 1.00 | 24.67 | D |
| ATOM | 8432 | CB | ARG D | 639 | 125.735 | 1.733 | 40.875 | 1.00 | 24.23 | D |
| ATOM | 8433 | CG | ARG D | 639 | 126.472 | 1.524 | 39.569 | 1.00 | 24.13 | D |
| ATOM | 8434 | CD | ARG D | 639 | 127.923 | 1.341 | 39.843 | 1.00 | 24.24 | D |
| ATOM | 8435 | NE | ARG D | 639 | 128.539 | 0.275 | 39.061 | 1.00 | 25.29 | D |
| ATOM | 8436 | CZ | ARG D | 639 | 129.345 | 0.488 | 38.027 | 1.00 | 26.83 | D |
| ATOM | 8437 | NH1 | ARG D | 639 | 129.609 | 1.735 | 37.658 | 1.00 | 22.95 | D |
| ATOM | 8438 | NH2 | ARG D | 639 | 129.909 | −0.538 | 37.387 | 1.00 | 27.61 | D |
| ATOM | 8439 | C | ARG D | 639 | 124.857 | 3.877 | 41.777 | 1.00 | 24.76 | D |
| ATOM | 8440 | O | ARG D | 639 | 123.942 | 3.540 | 42.519 | 1.00 | 24.70 | D |
| ATOM | 8441 | N | LEU D | 640 | 124.821 | 4.991 | 41.046 | 1.00 | 25.49 | D |
| ATOM | 8442 | CA | LEU D | 640 | 123.668 | 5.908 | 41.066 | 1.00 | 23.58 | D |
| ATOM | 8443 | CB | LEU D | 640 | 124.142 | 7.354 | 41.250 | 1.00 | 20.68 | D |
| ATOM | 8444 | CG | LEU D | 640 | 123.301 | 8.220 | 42.178 | 1.00 | 21.27 | D |
| ATOM | 8445 | CD1 | LEU D | 640 | 123.483 | 9.698 | 41.849 | 1.00 | 19.99 | D |
| ATOM | 8446 | CD2 | LEU D | 640 | 121.854 | 7.834 | 42.058 | 1.00 | 21.23 | D |
| ATOM | 8447 | C | LEU D | 640 | 122.888 | 5.800 | 39.753 | 1.00 | 23.09 | D |
| ATOM | 8448 | O | LEU D | 640 | 123.464 | 5.886 | 38.677 | 1.00 | 25.33 | D |
| ATOM | 8449 | N | SER D | 641 | 121.582 | 5.598 | 39.846 | 1.00 | 22.47 | D |
| ATOM | 8450 | CA | SER D | 641 | 120.741 | 5.498 | 38.668 | 1.00 | 21.80 | D |
| ATOM | 8451 | CB | SER D | 641 | 119.920 | 4.212 | 38.713 | 1.00 | 23.05 | D |
| ATOM | 8452 | OC | SER D | 641 | 118.628 | 4.399 | 38.170 | 1.00 | 26.20 | D |
| ATOM | 8453 | C | SER D | 641 | 119.837 | 6.712 | 38.706 | 1.00 | 22.79 | D |
| ATOM | 8454 | O | SER D | 641 | 119.697 | 7.344 | 39.743 | 1.00 | 21.44 | D |
| ATOM | 8455 | N | ASP D | 642 | 119.206 | 7.052 | 37.596 | 1.00 | 25.87 | D |
| ATOM | 8456 | CA | ASP D | 642 | 118.361 | 8.230 | 37.631 | 1.00 | 29.02 | D |
| ATOM | 8457 | CB | ASP D | 642 | 117.753 | 8.507 | 36.258 | 1.00 | 32.11 | D |
| ATOM | 8458 | CG | ASP D | 642 | 117.066 | 9.854 | 36.206 | 1.00 | 35.97 | D |
| ATOM | 8459 | OD1 | ASP D | 642 | 117.782 | 10.877 | 36.333 | 1.00 | 38.30 | D |
| ATOM | 8460 | OD2 | ASP D | 642 | 115.817 | 9.904 | 36.059 | 1.00 | 37.81 | D |
| ATOM | 8461 | C | ASP D | 642 | 117.247 | 8.077 | 38.655 | 1.00 | 28.71 | D |
| ATOM | 8462 | O | ASP D | 642 | 116.649 | 9.055 | 39.098 | 1.00 | 28.83 | D |
| ATOM | 8463 | N | LYS D | 643 | 116.975 | 6.847 | 39.047 | 1.00 | 29.24 | D |
| ATOM | 8464 | CA | LYS D | 643 | 115.903 | 6.626 | 39.989 | 1.00 | 29.96 | D |
| ATOM | 8465 | CB | LYS D | 643 | 114.760 | 5.934 | 39.241 | 1.00 | 31.36 | D |
| ATOM | 8466 | CG | LYS D | 643 | 113.626 | 5.382 | 40.076 | 1.00 | 36.57 | D |
| ATOM | 8467 | CD | LYS D | 643 | 112.491 | 4.792 | 39.183 | 1.00 | 38.95 | D |
| ATOM | 8468 | CE | LYS D | 643 | 111.899 | 5.844 | 38.222 | 1.00 | 40.27 | D |
| ATOM | 8469 | NZ | LYS D | 643 | 110.719 | 5.330 | 37.453 | 1.00 | 40.99 | D |
| ATOM | 8470 | C | LYS D | 643 | 116.325 | 5.831 | 41.222 | 1.00 | 29.02 | D |
| ATOM | 8471 | O | LYS D | 643 | 115.628 | 5.832 | 42.234 | 1.00 | 29.30 | D |
| ATOM | 8472 | N | PHE D | 644 | 117.483 | 5.187 | 41.160 | 1.00 | 27.45 | D |
| ATOM | 8473 | CA | PHE D | 644 | 117.924 | 4.370 | 42.276 | 1.00 | 25.90 | D |
| ATOM | 8474 | CB | PHE D | 644 | 117.822 | 2.889 | 41.918 | 1.00 | 23.62 | D |
| ATOM | 8475 | CG | PHE D | 644 | 116.428 | 2.416 | 41.670 | 1.00 | 22.94 | D |
| ATOM | 8476 | CD1 | PHE D | 644 | 116.097 | 1.784 | 40.480 | 1.00 | 23.53 | D |
| ATOM | 8477 | CD2 | PHE D | 644 | 115.448 | 2.567 | 42.632 | 1.00 | 21.03 | D |
| ATOM | 8478 | CE1 | PHE D | 644 | 114.807 | 1.309 | 40.266 | 1.00 | 21.81 | D |
| ATOM | 8479 | CE2 | PHE D | 644 | 114.164 | 2.094 | 42.416 | 1.00 | 18.29 | D |
| ATOM | 8480 | CZ | PHE D | 644 | 113.847 | 1.467 | 41.237 | 1.00 | 17.27 | D |
| ATOM | 8481 | C | PHE D | 644 | 119.337 | 4.618 | 42.735 | 1.00 | 26.43 | D |
| ATOM | 8482 | O | PHE D | 644 | 120.164 | 5.136 | 41.989 | 1.00 | 25.68 | D |
| ATOM | 8483 | N | LEU D | 645 | 119.589 | 4.238 | 43.988 | 1.00 | 27.14 | D |
| ATOM | 8484 | CA | LEU D | 645 | 120.911 | 4.318 | 44.608 | 1.00 | 26.31 | D |
| ATOM | 8485 | CB | LEU D | 645 | 120.914 | 5.234 | 45.828 | 1.00 | 27.08 | D |
| ATOM | 8486 | CG | LEU D | 645 | 122.217 | 5.194 | 46.640 | 1.00 | 27.94 | D |
| ATOM | 8487 | CD1 | LEU D | 645 | 123.290 | 5.975 | 45.918 | 1.00 | 29.78 | D |
| ATOM | 8488 | CD2 | LEU D | 645 | 121.994 | 5.766 | 48.028 | 1.00 | 27.68 | D |
| ATOM | 8489 | C | LEU D | 645 | 121.152 | 2.890 | 45.059 | 1.00 | 24.69 | D |
| ATOM | 8490 | O | LEU D | 645 | 120.758 | 2.514 | 46.146 | 1.00 | 26.35 | D |
| ATOM | 8491 | N | VAL D | 646 | 121.786 | 2.100 | 44.206 | 1.00 | 23.16 | D |
| ATOM | 8492 | CA | VAL D | 646 | 122.056 | 0.706 | 44.499 | 1.00 | 21.12 | D |
| ATOM | 8493 | CB | VAL D | 646 | 122.116 | −0.101 | 43.212 | 1.00 | 20.50 | D |
| ATOM | 8494 | CG1 | VAL D | 646 | 122.047 | −1.583 | 43.514 | 1.00 | 21.04 | D |
| ATOM | 8495 | CG2 | VAL D | 646 | 120.997 | 0.334 | 42.283 | 1.00 | 20.61 | D |
| ATOM | 8496 | C | VAL D | 646 | 123.384 | 0.523 | 45.211 | 1.00 | 20.80 | D |
| ATOM | 8497 | O | VAL D | 646 | 124.375 | 1.159 | 44.857 | 1.00 | 21.52 | D |
| ATOM | 8498 | N | SER D | 647 | 123.408 | −0.356 | 46.208 | 1.00 | 20.76 | D |
| ATOM | 8499 | CA | SER D | 647 | 124.637 | −0.643 | 46.936 | 1.00 | 19.72 | D |
| ATOM | 8500 | CB | SER D | 647 | 124.706 | 0.166 | 48.231 | 1.00 | 19.38 | D |
| ATOM | 8501 | OG | SER D | 647 | 123.594 | −0.100 | 49.071 | 1.00 | 20.83 | D |
| ATOM | 8502 | C | SER D | 647 | 124.723 | −2.140 | 47.249 | 1.00 | 19.78 | D |
| ATOM | 8503 | O | SER D | 647 | 123.694 | −2.816 | 47.373 | 1.00 | 18.23 | D |
| ATOM | 8504 | N | ALA D | 648 | 125.950 | −2.654 | 47.344 | 1.00 | 18.94 | D |
| ATOM | 8505 | CA | ALA D | 648 | 126.175 | −4.060 | 47.646 | 1.00 | 19.09 | D |
| ATOM | 8506 | CB | ALA D | 648 | 126.463 | −4.850 | 46.377 | 1.00 | 15.72 | D |
| ATOM | 8507 | C | ALA D | 648 | 127.337 | −4.180 | 48.618 | 1.00 | 20.74 | D |
| ATOM | 8508 | O | ALA D | 648 | 128.279 | −3.399 | 48.547 | 1.00 | 22.53 | D |
| ATOM | 8509 | N | ALA D | 649 | 127.253 | −5.148 | 49.533 | 1.00 | 21.16 | D |
| ATOM | 8510 | CA | ALA D | 649 | 128.301 | −5.372 | 50.514 | 1.00 | 20.36 | D |
| ATOM | 8511 | CB | ALA D | 649 | 127.774 | −5.097 | 51.911 | 1.00 | 23.59 | D |
| ATOM | 8512 | C | ALA D | 649 | 128.912 | −6.772 | 50.457 | 1.00 | 20.64 | D |
| ATOM | 8513 | O | ALA D | 649 | 128.453 | −7.663 | 49.723 | 1.00 | 19.52 | D |
| ATOM | 8514 | N | ALA D | 650 | 129.951 | −6.944 | 51.271 | 1.00 | 18.79 | D |
| ATOM | 8515 | CA | ALA D | 650 | 130.703 | −8.192 | 51.372 | 1.00 | 17.83 | D |
| ATOM | 8516 | CB | ALA D | 650 | 131.839 | −8.012 | 52.334 | 1.00 | 17.25 | D |
| ATOM | 8517 | C | ALA D | 650 | 129.889 | −9.409 | 51.786 | 1.00 | 17.38 | D |
| ATOM | 8518 | O | ALA D | 650 | 130.337 | −10.545 | 51.626 | 1.00 | 16.75 | D |
| ATOM | 8519 | N | ASP D | 651 | 128.699 | −9.190 | 52.329 | 1.00 | 18.32 | D |
| ATOM | 8520 | CA | ASP D | 651 | 127.880 | −10.323 | 52.748 | 1.00 | 19.29 | D |
| ATOM | 8521 | CB | ASP D | 651 | 126.982 | −9.930 | 53.920 | 1.00 | 21.96 | D |
| ATOM | 8522 | CG | ASP D | 651 | 126.027 | −8.804 | 53.568 | 1.00 | 26.40 | D |
| ATOM | 8523 | OD1 | ASP D | 651 | 125.508 | −8.816 | 52.425 | 1.00 | 28.33 | D |
| ATOM | 8524 | OD2 | ASP D | 651 | 125.781 | −7.924 | 54.436 | 1.00 | 25.72 | D |
| ATOM | 8525 | C | ASP D | 651 | 127.026 | −10.856 | 51.609 | 1.00 | 19.07 | D |
| ATOM | 8526 | O | ASP D | 651 | 126.244 | −11.789 | 51.787 | 1.00 | 20.37 | D |
| ATOM | 8527 | N | GLY D | 652 | 127.164 | −10.256 | 50.437 | 1.00 | 19.11 | D |
| ATOM | 8528 | CA | GLY D | 652 | 126.370 | −10.711 | 49.315 | 1.00 | 18.71 | D |
| ATOM | 8529 | C | GLY D | 652 | 125.024 | −10.018 | 49.213 | 1.00 | 18.78 | D |
| ATOM | 8530 | O | GLY D | 652 | 124.142 | −10.475 | 48.479 | 1.00 | 17.99 | D |
| ATOM | 8531 | N | SER D | 653 | 124.841 | −8.926 | 49.950 | 1.00 | 20.24 | D |
| ATOM | 8532 | CA | SER D | 653 | 123.575 | −8.198 | 49.864 | 1.00 | 21.58 | D |
| ATOM | 8533 | CB | SER D | 653 | 123.133 | −7.678 | 51.236 | 1.00 | 21.54 | D |
| ATOM | 8534 | OG | SER D | 653 | 124.037 | −6.731 | 51.772 | 1.00 | 22.98 | D |
| ATOM | 8535 | C | SER D | 653 | 123.665 | −7.033 | 48.881 | 1.00 | 22.14 | D |
| ATOM | 8536 | O | SER D | 653 | 124.742 | −6.510 | 48.598 | 1.00 | 21.79 | D |
| ATOM | 8537 | N | ILE D | 654 | 122.515 | −6.652 | 48.350 | 1.00 | 23.25 | D |
| ATOM | 8538 | CA | ILE D | 654 | 122.418 | −5.554 | 47.409 | 1.00 | 25.80 | D |
| ATOM | 8539 | CB | ILE D | 654 | 122.165 | −6.055 | 45.982 | 1.00 | 26.21 | D |
| ATOM | 8540 | CG2 | ILE D | 654 | 122.297 | −4.918 | 45.003 | 1.00 | 26.27 | D |
| ATOM | 8541 | CG1 | ILE D | 654 | 123.154 | −7.158 | 45.616 | 1.00 | 28.16 | D |
| ATOM | 8542 | CD1 | ILE D | 654 | 122.870 | −7.785 | 44.254 | 1.00 | 28.37 | D |
| ATOM | 8543 | C | ILE D | 654 | 121.197 | −4.754 | 47.836 | 1.00 | 26.75 | D |
| ATOM | 8544 | O | ILE D | 654 | 120.122 | −5.323 | 48.064 | 1.00 | 27.01 | D |
| ATOM | 8545 | N | ARG D | 655 | 121.346 | −3.443 | 47.951 | 1.00 | 27.07 | D |
| ATOM | 8546 | CA | ARG D | 655 | 120.216 | −2.626 | 48.356 | 1.00 | 27.67 | D |
| ATOM | 8547 | CB | ARG D | 655 | 120.467 | −2.029 | 49.737 | 1.00 | 28.04 | D |
| ATOM | 8548 | CG | ARG D | 655 | 120.592 | −3.062 | 50.832 | 1.00 | 29.00 | D |
| ATOM | 8549 | CD | ARG D | 655 | 119.901 | −2.605 | 52.103 | 1.00 | 31.71 | D |
| ATOM | 8550 | NE | ARG D | 655 | 118.574 | −3.199 | 52.240 | 1.00 | 33.84 | D |
| ATOM | 8551 | CZ | ARG D | 655 | 118.328 | −4.314 | 52.914 | 1.00 | 33.80 | D |
| ATOM | 8552 | NH1 | ARG D | 655 | 119.314 | −4.960 | 53.527 | 1.00 | 33.02 | D |
| ATOM | 8553 | NH2 | ARG D | 655 | 117.094 | −4.787 | 52.953 | 1.00 | 36.00 | D |
| ATOM | 8554 | C | ARG D | 655 | 119.903 | −1.524 | 47.358 | 1.00 | 27.31 | D |
| ATOM | 8555 | O | ARG D | 655 | 120.797 | −0.985 | 46.706 | 1.00 | 26.04 | D |
| ATOM | 8556 | N | GLY D | 656 | 118.620 | −1.203 | 47.237 | 1.00 | 27.56 | D |
| ATOM | 8557 | CA | GLY D | 656 | 118.206 | −0.164 | 46.315 | 1.00 | 28.15 | D |
| ATOM | 8558 | C | GLY D | 656 | 117.482 | 0.916 | 47.087 | 1.00 | 27.66 | D |
| ATOM | 8559 | O | GLY D | 656 | 116.514 | 0.621 | 47.775 | 1.00 | 28.41 | D |
| ATOM | 8560 | N | TRP D | 657 | 117.944 | 2.158 | 46.996 | 1.00 | 27.78 | D |
| ATOM | 8561 | CA | TRP D | 657 | 117.299 | 3.256 | 47.715 | 1.00 | 28.29 | D |
| ATOM | 8562 | CB | TRP D | 657 | 118.296 | 3.913 | 48.699 | 1.00 | 26.47 | D |
| ATOM | 8563 | CG | TRP D | 657 | 119.251 | 2.947 | 49.419 | 1.00 | 24.99 | D |
| ATOM | 8564 | CD2 | TRP D | 657 | 119.136 | 2.448 | 50.768 | 1.00 | 23.38 | D |
| ATOM | 8565 | CE2 | TRP D | 657 | 120.215 | 1.554 | 50.972 | 1.00 | 22.65 | D |
| ATOM | 8566 | CE3 | TRP D | 657 | 118.226 | 2.664 | 51.818 | 1.00 | 22.66 | D |
| ATOM | 8567 | CD1 | TRP D | 657 | 120.369 | 2.352 | 48.891 | 1.00 | 22.06 | D |
| ATOM | 8568 | NE1 | TRP D | 657 | 120.947 | 1.520 | 49.815 | 1.00 | 21.92 | D |
| ATOM | 8569 | CZ2 | TRP D | 657 | 120.407 | 0.875 | 52.180 | 1.00 | 20.78 | D |
| ATOM | 8570 | CZ3 | TRP D | 657 | 118.423 | 1.979 | 53.026 | 1.00 | 22.09 | D |
| ATOM | 8571 | CR2 | TRP D | 657 | 119.505 | 1.098 | 53.187 | 1.00 | 22.26 | D |
| ATOM | 8572 | C | TRP D | 657 | 116.777 | 4.313 | 46.705 | 1.00 | 29.51 | D |
| ATOM | 8573 | O | TRP D | 657 | 117.304 | 4.422 | 45.592 | 1.00 | 29.88 | D |
| ATOM | 8574 | N | ASP D | 658 | 115.744 | 5.076 | 47.075 | 1.00 | 29.21 | D |
| ATOM | 8575 | CA | ASP D | 658 | 115.223 | 6.104 | 46.174 | 1.00 | 29.96 | D |
| ATOM | 8576 | CB | ASP D | 658 | 113.977 | 6.790 | 46.757 | 1.00 | 28.48 | D |
| ATOM | 8577 | CG | ASP D | 658 | 113.460 | 7.923 | 45.868 | 1.00 | 28.43 | D |
| ATOM | 8578 | OD1 | ASP D | 658 | 112.888 | 7.650 | 44.793 | 1.00 | 28.91 | D |
| ATOM | 8579 | OD2 | ASP D | 658 | 113.628 | 9.101 | 46.236 | 1.00 | 27.59 | D |
| ATOM | 8580 | C | ASP D | 658 | 116.354 | 7.113 | 46.026 | 1.00 | 31.50 | D |
| ATOM | 8581 | O | ASP D | 658 | 116.978 | 7.503 | 47.017 | 1.00 | 32.96 | D |
| ATOM | 8582 | N | ALA D | 659 | 116.621 | 7.526 | 44.791 | 1.00 | 31.88 | D |
| ATOM | 8583 | CA | ALA D | 659 | 117.694 | 8.468 | 44.499 | 1.00 | 32.44 | D |
| ATOM | 8584 | CB | ALA D | 659 | 117.811 | 8.649 | 43.010 | 1.00 | 32.07 | D |
| ATOM | 8585 | C | ALA D | 659 | 117.541 | 9.829 | 45.162 | 1.00 | 33.56 | D |
| ATOM | 8586 | O | ALA D | 659 | 118.463 | 10.634 | 45.141 | 1.00 | 34.05 | D |
| ATOM | 8587 | N | ASN D | 660 | 116.387 | 10.091 | 45.761 | 1.00 | 36.44 | D |
| ATOM | 8588 | CA | ASN D | 660 | 116.155 | 11.379 | 46.400 | 1.00 | 37.59 | D |
| ATOM | 8589 | CB | ASN D | 660 | 115.061 | 12.130 | 45.645 | 1.00 | 39.91 | D |
| ATOM | 8590 | CG | ASN D | 660 | 114.872 | 13.542 | 46.149 | 1.00 | 43.39 | D |
| ATOM | 8591 | OD1 | ASN D | 660 | 114.382 | 13.764 | 47.264 | 1.00 | 44.43 | D |
| ATOM | 8592 | ND2 | ASN D | 660 | 115.274 | 14.515 | 45.334 | 1.00 | 45.95 | D |
| ATOM | 8593 | C | ASN D | 660 | 115.791 | 11.248 | 47.876 | 1.00 | 36.76 | D |
| ATOM | 8594 | O | ASN D | 660 | 116.233 | 12.050 | 48.689 | 1.00 | 38.43 | D |
| ATOM | 8595 | N | ASP D | 661 | 114.991 | 10.239 | 48.214 | 1.00 | 35.26 | D |
| ATOM | 8596 | CA | ASP D | 661 | 114.566 | 9.984 | 49.592 | 1.00 | 33.16 | D |
| ATOM | 8597 | CB | ASP D | 661 | 113.165 | 9.393 | 49.620 | 1.00 | 34.45 | D |
| ATOM | 8598 | CG | ASP D | 661 | 112.144 | 10.331 | 49.104 | 1.00 | 38.11 | D |
| ATOM | 8599 | OD1 | ASP D | 661 | 112.280 | 11.540 | 49.419 | 1.00 | 40.95 | D |
| ATOM | 8600 | OD2 | ASP D | 661 | 111.200 | 9.876 | 48.409 | 1.00 | 39.95 | D |
| ATOM | 8601 | C | ASP D | 661 | 115.458 | 8.980 | 50.292 | 1.00 | 31.13 | D |
| ATOM | 8602 | O | ASP D | 661 | 115.569 | 9.004 | 51.505 | 1.00 | 31.79 | D |
| ATOM | 8603 | N | TYR D | 662 | 116.023 | 8.062 | 49.517 | 1.00 | 26.92 | D |
| ATOM | 8604 | CA | TYR D | 662 | 116.885 | 7.016 | 50.027 | 1.00 | 25.03 | D |
| ATOM | 8605 | CB | TYR D | 662 | 118.028 | 7.615 | 50.876 | 1.00 | 23.52 | D |
| ATOM | 8606 | CG | TYR D | 662 | 118.839 | 8.622 | 50.075 | 1.00 | 24.99 | D |
| ATOM | 8607 | CD1 | TYR D | 662 | 119.614 | 8.201 | 48.999 | 1.00 | 23.45 | D |
| ATOM | 8608 | CE1 | TYR D | 662 | 120.257 | 9.099 | 48.181 | 1.00 | 23.80 | D |
| ATOM | 8609 | CD2 | TYR D | 662 | 118.739 | 9.996 | 50.317 | 1.00 | 23.15 | D |
| ATOM | 8610 | CE2 | TYR D | 662 | 119.384 | 10.914 | 49.497 | 1.00 | 24.08 | D |
| ATOM | 8611 | CZ | TYR D | 662 | 120.143 | 10.459 | 48.424 | 1.00 | 25.80 | D |
| ATOM | 8612 | OH | TYR D | 662 | 120.770 | 11.367 | 47.581 | 1.00 | 25.60 | D |
| ATOM | 8613 | C | TYR D | 662 | 116.090 | 5.979 | 50.808 | 1.00 | 24.80 | D |
| ATOM | 8614 | O | TYR D | 662 | 116.663 | 5.210 | 51.590 | 1.00 | 25.89 | D |
| ATOM | 8615 | N | SER D | 663 | 114.777 | 5.946 | 50.575 | 1.00 | 21.66 | D |
| ATOM | 8616 | CA | SER D | 663 | 113.886 | 4.981 | 51.228 | 1.00 | 21.07 | D |
| ATOM | 8617 | CB | SER D | 663 | 112.432 | 5.460 | 51.165 | 1.00 | 20.52 | D |
| ATOM | 8618 | OG | SER D | 663 | 111.987 | 5.642 | 49.838 | 1.00 | 19.13 | D |
| ATOM | 8619 | C | SER D | 663 | 113.996 | 3.610 | 50.564 | 1.00 | 20.63 | D |
| ATOM | 8620 | O | SER D | 663 | 114.182 | 3.530 | 49.357 | 1.00 | 20.77 | D |
| ATOM | 8621 | N | ARG D | 664 | 113.901 | 2.537 | 51.353 | 1.00 | 21.12 | D |
| ATOM | 8622 | CA | ARG D | 664 | 114.022 | 1.180 | 50.819 | 1.00 | 20.22 | D |
| ATOM | 8623 | CB | ARG D | 664 | 113.564 | 0.125 | 51.849 | 1.00 | 22.66 | D |
| ATOM | 8624 | CG | ARG D | 664 | 114.575 | −0.302 | 52.980 | 1.00 | 25.13 | D |
| ATOM | 8625 | CD | ARG D | 664 | 114.860 | 0.821 | 53.987 | 1.00 | 27.71 | D |
| ATOM | 8626 | NE | ARG D | 664 | 115.573 | 0.440 | 55.219 | 1.00 | 29.68 | D |
| ATOM | 8627 | CZ | ARG D | 664 | 116.626 | −0.373 | 55.291 | 1.00 | 31.69 | D |
| ATOM | 8628 | NH1 | ARG D | 664 | 117.114 | −0.935 | 54.200 | 1.00 | 34.72 | D |
| ATOM | 8629 | NH2 | ARG D | 664 | 117.226 | −0.597 | 56.456 | 1.00 | 30.46 | D |
| ATOM | 8630 | C | ARG D | 664 | 113.196 | 1.037 | 49.552 | 1.00 | 18.91 | D |
| ATOM | 8631 | O | ARG D | 664 | 112.013 | 1.393 | 49.525 | 1.00 | 19.59 | D |
| ATOM | 8632 | N | LYS D | 665 | 113.833 | 0.544 | 48.496 | 1.00 | 17.43 | D |
| ATOM | 8633 | CA | LYS D | 665 | 113.144 | 0.320 | 47.238 | 1.00 | 16.40 | D |
| ATOM | 8634 | CB | LYS D | 665 | 113.729 | 1.204 | 46.148 | 1.00 | 14.06 | D |
| ATOM | 8635 | CG | LYS D | 665 | 113.132 | 2.582 | 46.190 | 1.00 | 13.78 | D |
| ATOM | 8636 | CD | LYS D | 665 | 111.661 | 2.434 | 45.960 | 1.00 | 14.84 | D |
| ATOM | 8637 | CE | LYS D | 665 | 110.875 | 3.713 | 46.115 | 1.00 | 14.42 | D |
| ATOM | 8638 | NZ | LYS D | 665 | 109.447 | 3.311 | 45.873 | 1.00 | 17.38 | D |
| ATOM | 8639 | C | LYS D | 665 | 113.202 | −1.149 | 46.868 | 1.00 | 15.50 | D |
| ATOM | 8640 | O | LYS D | 665 | 112.216 | −1.709 | 46.413 | 1.00 | 18.21 | D |
| ATOM | 8641 | N | PHE D | 666 | 114.352 | −1.778 | 47.069 | 1.00 | 15.94 | D |
| ATOM | 8642 | CA | PHE D | 666 | 114.492 | −3.207 | 46.810 | 1.00 | 16.38 | D |
| ATOM | 8643 | CB | PHE D | 666 | 114.453 | −3.531 | 45.291 | 1.00 | 12.81 | D |
| ATOM | 8644 | CG | PHE D | 666 | 115.613 | −2.980 | 44.477 | 1.00 | 13.72 | D |
| ATOM | 8645 | CD1 | PHE D | 666 | 116.914 | −3.428 | 44.676 | 1.00 | 14.32 | D |
| ATOM | 8646 | CD2 | PHE D | 666 | 115.386 | −2.051 | 43.460 | 1.00 | 16.32 | D |
| ATOM | 8647 | CE1 | PHE D | 666 | 117.963 | −2.962 | 43.881 | 1.00 | 12.43 | D |
| ATOM | 8648 | CE2 | PEE D | 666 | 116.433 | −1.579 | 42.659 | 1.00 | 14.70 | D |
| ATOM | 8649 | CZ | PHE D | 666 | 117.716 | −2.039 | 42.873 | 1.00 | 15.14 | D |
| ATOM | 8650 | C | PHE D | 666 | 115.764 | −3.767 | 47.482 | 1.00 | 17.88 | D |
| ATOM | 8651 | O | PHE D | 666 | 116.618 | −3.017 | 47.971 | 1.00 | 18.62 | D |
| ATOM | 8652 | N | SER D | 667 | 115.869 | −5.088 | 47.533 | 1.00 | 19.07 | D |
| ATOM | 8653 | CA | SER D | 667 | 117.021 | −5.736 | 48.127 | 1.00 | 20.16 | D |
| ATOM | 8654 | CE | SER D | 667 | 116.971 | −5.646 | 49.661 | 1.00 | 21.18 | D |
| ATOM | 8655 | OG | SER D | 667 | 115.778 | −6.192 | 50.188 | 1.00 | 21.36 | D |
| ATOM | 8656 | C | SER D | 667 | 117.089 | −7.188 | 47.675 | 1.00 | 19.78 | D |
| ATOM | 8657 | O | SER D | 667 | 116.093 | −7.907 | 47.662 | 1.00 | 18.38 | D |
| ATOM | 8658 | N | TYR D | 668 | 118.280 | −7.596 | 47.274 | 1.00 | 20.33 | D |
| ATOM | 8659 | CA | TYR D | 668 | 118.499 | −8.946 | 46.807 | 1.00 | 21.29 | D |
| ATOM | 8660 | CE | TYR D | 668 | 118.756 | −8.936 | 45.293 | 1.00 | 21.18 | D |
| ATOM | 8661 | CG | TYR D | 668 | 117.595 | −8.369 | 44.470 | 1.00 | 20.55 | D |
| ATOM | 8662 | CD1 | TYR D | 668 | 116.495 | −9.171 | 44.114 | 1.00 | 18.14 | D |
| ATOM | 8663 | CE1 | TYR D | 668 | 115.423 | −8.647 | 43.403 | 1.00 | 18.24 | D |
| ATOM | 8664 | CD2 | TYR D | 668 | 117.584 | −7.020 | 44.084 | 1.00 | 18.54 | D |
| ATOM | 8665 | CE2 | TYR D | 668 | 116.522 | −6.484 | 43.379 | 1.00 | 18.25 | D |
| ATOM | 8666 | CZ | TYR D | 668 | 115.437 | −7.293 | 43.044 | 1.00 | 19.79 | D |
| ATOM | 8667 | OH | TYR D | 668 | 114.343 | −6.732 | 42.410 | 1.00 | 18.58 | D |
| ATOM | 8668 | C | TYR D | 668 | 119.709 | −9.445 | 47.560 | 1.00 | 21.68 | D |
| ATOM | 8669 | O | TYR D | 668 | 120.525 | −8.650 | 48.008 | 1.00 | 24.28 | D |
| ATOM | 8670 | N | HIS D | 669 | 119.802 | −10.757 | 47.718 | 1.00 | 23.30 | D |
| ATOM | 8671 | CA | HIS D | 669 | 120.904 | −11.398 | 48.413 | 1.00 | 23.12 | D |
| ATOM | 8672 | CB | HIS D | 669 | 120.433 | −11.895 | 49.771 | 1.00 | 23.33 | D |
| ATOM | 8673 | CG | HIS D | 669 | 121.343 | −11.528 | 50.897 | 1.00 | 26.89 | D |
| ATOM | 8674 | CD2 | HIS D | 669 | 121.187 | −10.654 | 51.921 | 1.00 | 25.28 | D |
| ATOM | 8675 | ND1 | HIS D | 669 | 122.608 | −12.060 | 51.034 | 1.00 | 26.65 | D |
| ATOM | 8676 | CE1 | HIS D | 669 | 123.193 | −11.529 | 52.093 | 1.00 | 27.24 | D |
| ATOM | 8677 | NE2 | HIS D | 669 | 122.353 | −10.673 | 52.649 | 1.00 | 27.53 | D |
| ATOM | 8678 | C | HIS D | 669 | 121.274 | −12.587 | 47.535 | 1.00 | 24.51 | D |
| ATOM | 8679 | O | HIS D | 669 | 120.399 | −13.313 | 47.036 | 1.00 | 24.95 | D |
| ATOM | 8680 | N | HIS D | 670 | 122.569 | −12.779 | 47.335 | 1.00 | 24.99 | D |
| ATOM | 8681 | CA | HIS D | 670 | 123.045 | −13.882 | 46.521 | 1.00 | 25.59 | D |
| ATOM | 8682 | CE | HIS D | 670 | 124.501 | −13.642 | 46.121 | 1.00 | 26.37 | D |
| ATOM | 8683 | CG | HIS D | 670 | 124.666 | −12.905 | 44.823 | 1.00 | 29.01 | D |
| ATOM | 8684 | CD2 | HIS D | 670 | 125.637 | −12.971 | 43.881 | 1.00 | 27.78 | D |
| ATOM | 8685 | ND1 | HIS D | 670 | 123.779 | −11.942 | 44.387 | 1.00 | 28.88 | D |
| ATOM | 8686 | CE1 | HIS D | 670 | 124.199 | −11.450 | 43.236 | 1.00 | 26.47 | D |
| ATOM | 8687 | NE2 | HIS D | 670 | 125.324 | −12.055 | 42.907 | 1.00 | 25.99 | D |
| ATOM | 8688 | C | HIS D | 670 | 122.918 | −15.176 | 47.326 | 1.00 | 27.14 | D |
| ATOM | 8689 | O | HIS D | 670 | 123.583 | −15.346 | 48.362 | 1.00 | 25.08 | D |
| ATOM | 8690 | N | THR D | 671 | 122.059 | −16.080 | 46.840 | 1.00 | 28.13 | D |
| ATOM | 8691 | CA | THR D | 671 | 121.796 | −17.381 | 47.473 | 1.00 | 27.79 | D |
| ATOM | 8692 | CB | THR D | 671 | 121.180 | −18.369 | 46.454 | 1.00 | 26.12 | D |
| ATOM | 8693 | OG1 | THR D | 671 | 119.789 | −18.069 | 46.289 | 1.00 | 23.95 | D |
| ATOM | 8694 | CG2 | THR D | 671 | 121.312 | −19.791 | 46.930 | 1.00 | 26.83 | D |
| ATOM | 8695 | C | THR D | 671 | 122.992 | −18.034 | 48.162 | 1.00 | 29.31 | D |
| ATOM | 8696 | O | THR D | 671 | 122.854 | −18.547 | 49.271 | 1.00 | 29.25 | D |
| ATOM | 8697 | N | ASN D | 672 | 124.156 | −18.025 | 47.516 | 1.00 | 31.23 | D |
| ATOM | 8698 | CA | ASN D | 672 | 125.354 | −18.627 | 48.116 | 1.00 | 33.42 | D |
| ATOM | 8699 | CB | ASN D | 672 | 126.498 | −18.738 | 47.089 | 1.00 | 35.89 | D |
| ATOM | 8700 | CG | ASN D | 672 | 126.323 | −19.911 | 46.105 | 1.00 | 40.95 | D |
| ATOM | 8701 | OD1 | ASN D | 672 | 125.241 | −20.116 | 45.535 | 1.00 | 42.75 | D |
| ATOM | 8702 | ND2 | ASN D | 672 | 127.406 | −20.672 | 45.887 | 1.00 | 42.55 | D |
| ATOM | 8703 | C | ASN D | 672 | 125.861 | −17.813 | 49.317 | 1.00 | 33.48 | D |
| ATOM | 8704 | O | ASN D | 672 | 126.666 | −18.302 | 50.105 | 1.00 | 35.72 | D |
| ATOM | 8705 | N | LEU D | 673 | 125.405 | −16.574 | 49.455 | 1.00 | 31.40 | D |
| ATOM | 8706 | CA | LEU D | 673 | 125.864 | −15.722 | 50.549 | 1.00 | 30.75 | D |
| ATOM | 8707 | CB | LEU D | 673 | 125.631 | −16.410 | 51.901 | 1.00 | 30.83 | D |
| ATOM | 8708 | CG | LEU D | 673 | 124.165 | −16.514 | 52.344 | 1.00 | 31.99 | D |
| ATOM | 8709 | CD1 | LEU D | 673 | 124.059 | −17.284 | 53.649 | 1.00 | 32.25 | D |
| ATOM | 8710 | CD2 | LEU D | 673 | 123.580 | −15.115 | 52.510 | 1.00 | 33.43 | D |
| ATOM | 8711 | C | LEU D | 673 | 127.346 | −15.337 | 50.420 | 1.00 | 28.49 | D |
| ATOM | 8712 | O | LEU D | 673 | 128.045 | −15.214 | 51.429 | 1.00 | 29.31 | D |
| ATOM | 8713 | N | SER D | 674 | 127.812 | −15.145 | 49.185 | 1.00 | 24.89 | D |
| ATOM | 8714 | CA | SER D | 674 | 129.201 | −14.771 | 48.911 | 1.00 | 23.72 | D |
| ATOM | 8715 | CB | SER D | 674 | 129.764 | −15.608 | 47.764 | 1.00 | 23.54 | D |
| ATOM | 8716 | OG | SER D | 674 | 128.969 | −15.454 | 46.598 | 1.00 | 27.66 | D |
| ATOM | 8717 | C | SER D | 674 | 129.289 | −13.287 | 48.546 | 1.00 | 22.55 | D |
| ATOM | 8718 | O | SER D | 674 | 128.336 | −12.721 | 48.035 | 1.00 | 22.93 | D |
| ATOM | 8719 | N | ALA D | 675 | 130.431 | −12.657 | 48.796 | 1.00 | 21.17 | D |
| ATOM | 8720 | CA | ALA D | 675 | 130.580 | −11.240 | 48.498 | 1.00 | 20.35 | D |
| ATOM | 8721 | CB | ALA D | 675 | 132.002 | −10.779 | 48.814 | 1.00 | 16.29 | D |
| ATOM | 8722 | C | ALA D | 675 | 130.215 | −10.836 | 47.075 | 1.00 | 20.97 | D |
| ATOM | 8723 | O | ALA D | 675 | 130.528 | −11.527 | 46.095 | 1.00 | 22.46 | D |
| ATOM | 8724 | N | ILE D | 676 | 129.538 | −9.703 | 46.972 | 1.00 | 21.38 | D |
| ATOM | 8725 | CA | ILE D | 676 | 129.180 | −9.194 | 45.667 | 1.00 | 23.44 | D |
| ATOM | 8726 | CB | ILE D | 676 | 128.051 | −8.132 | 45.734 | 1.00 | 24.56 | D |
| ATOM | 8727 | CG2 | ILE D | 676 | 127.897 | −7.484 | 44.377 | 1.00 | 23.13 | D |
| ATOM | 8728 | CG1 | ILE D | 676 | 126.727 | −8.776 | 46.197 | 1.00 | 25.69 | D |
| ATOM | 8729 | CD1 | ILE D | 676 | 126.316 | −10.018 | 45.386 | 1.00 | 25.28 | D |
| ATOM | 8730 | C | ILE D | 676 | 130.470 | −8.526 | 45.202 | 1.00 | 23.09 | D |
| ATOM | 8731 | O | ILE D | 676 | 130.971 | −7.613 | 45.853 | 1.00 | 23.97 | D |
| ATOM | 8732 | N | THR D | 677 | 131.012 | −9.000 | 44.091 | 1.00 | 21.72 | D |
| ATOM | 8733 | CA | THR D | 677 | 132.250 | −8.474 | 43.554 | 1.00 | 21.86 | D |
| ATOM | 8734 | CB | THR D | 677 | 133.096 | −9.589 | 42.896 | 1.00 | 25.37 | D |
| ATOM | 8735 | OG1 | THR D | 677 | 133.515 | −10.542 | 43.884 | 1.00 | 25.75 | D |
| ATOM | 8736 | CG2 | THR D | 677 | 134.328 | −8.977 | 42.219 | 1.00 | 27.86 | D |
| ATOM | 8737 | C | THR D | 677 | 132.001 | −7.420 | 42.488 | 1.00 | 20.83 | D |
| ATOM | 8738 | O | THR D | 677 | 132.880 | −6.605 | 42.208 | 1.00 | 17.53 | D |
| ATOM | 8739 | N | THR D | 678 | 130.818 | −7.457 | 41.876 | 1.00 | 20.52 | D |
| ATOM | 8740 | CA | THR D | 678 | 130.492 | −6.512 | 40.821 | 1.00 | 19.25 | D |
| ATOM | 8741 | CB | THR D | 678 | 131.294 | −6.864 | 39.512 | 1.00 | 20.24 | D |
| ATOM | 8742 | OG1 | THR D | 678 | 131.093 | −5.842 | 38.533 | 1.00 | 19.49 | D |
| ATOM | 8743 | CG2 | THR D | 678 | 130.834 | −8.193 | 38.927 | 1.00 | 17.27 | D |
| ATOM | 8744 | C | THR D | 678 | 129.001 | −6.490 | 40.495 | 1.00 | 18.40 | D |
| ATOM | 8745 | O | THR D | 678 | 128.308 | −7.497 | 40.627 | 1.00 | 17.73 | D |
| ATOM | 8746 | N | PHE D | 679 | 128.521 | −5.336 | 40.044 | 1.00 | 19.50 | D |
| ATOM | 8747 | CA | PHE D | 679 | 127.119 | −5.179 | 39.653 | 1.00 | 20.10 | D |
| ATOM | 8748 | CB | PHE D | 679 | 126.212 | −5.151 | 40.896 | 1.00 | 19.62 | D |
| ATOM | 8749 | CG | PHE D | 679 | 126.072 | −3.794 | 41.546 | 1.00 | 20.58 | D |
| ATOM | 8750 | CD1 | PHE D | 679 | 125.187 | −2.838 | 41.036 | 1.00 | 19.81 | D |
| ATOM | 8751 | CD2 | PHE D | 679 | 126.786 | −3.493 | 42.708 | 1.00 | 22.11 | D |
| ATOM | 8752 | CE1 | PHE D | 679 | 125.008 | −1.595 | 41.679 | 1.00 | 19.56 | D |
| ATOM | 8753 | CE2 | PHE D | 679 | 126.622 | −2.255 | 43.369 | 1.00 | 21.42 | D |
| ATOM | 8754 | CZ | PHE D | 679 | 125.730 | −1.305 | 42.855 | 1.00 | 20.49 | D |
| ATOM | 8755 | C | PHE D | 679 | 126.967 | −3.908 | 38.836 | 1.00 | 18.56 | D |
| ATOM | 8756 | O | PHE D | 679 | 127.824 | −3.040 | 38.902 | 1.00 | 18.64 | D |
| ATOM | 8757 | N | TYR D | 680 | 125.900 | −3.826 | 38.038 | 1.00 | 18.73 | D |
| ATOM | 8758 | CA | TYR D | 680 | 125.610 | −2.640 | 37.216 | 1.00 | 18.62 | D |
| ATOM | 8759 | CB | TYR D | 680 | 126.140 | −2.818 | 35.776 | 1.00 | 19.64 | D |
| ATOM | 8760 | CG | TYR D | 680 | 126.416 | −1.498 | 35.070 | 1.00 | 21.94 | D |
| ATOM | 8761 | CD1 | TYR D | 680 | 127.503 | −0.704 | 35.447 | 1.00 | 21.42 | D |
| ATOM | 8762 | CE1 | TYR D | 680 | 127.691 | 0.578 | 34.917 | 1.00 | 21.58 | D |
| ATOM | 8763 | CD2 | TYR D | 680 | 125.525 | −0.982 | 34.123 | 1.00 | 21.80 | D |
| ATOM | 8764 | CE2 | TYR D | 680 | 125.702 | 0.304 | 33.585 | 1.00 | 21.45 | D |
| ATOM | 8765 | CZ | TYR D | 680 | 126.780 | 1.083 | 33.992 | 1.00 | 21.61 | D |
| ATOM | 8766 | OH | TYR D | 680 | 126.891 | 2.384 | 33.541 | 1.00 | 21.38 | D |
| ATOM | 8767 | C | TYR D | 680 | 124.087 | −2.429 | 37.194 | 1.00 | 18.89 | D |
| ATOM | 8768 | O | TYR D | 680 | 123.326 | −3.394 | 37.244 | 1.00 | 21.29 | D |
| ATOM | 8769 | N | VAL D | 681 | 123.635 | −1.182 | 37.148 | 1.00 | 18.55 | D |
| ATOM | 8770 | CA | VAL D | 681 | 122.202 | −0.952 | 37.112 | 1.00 | 20.06 | D |
| ATOM | 8771 | CB | VAL D | 681 | 121.588 | −0.612 | 38.502 | 1.00 | 19.46 | D |
| ATOM | 8772 | CG1 | VAL D | 681 | 121.748 | −1.770 | 39.427 | 1.00 | 23.82 | D |
| ATOM | 8773 | CG2 | VAL D | 681 | 122.219 | 0.634 | 39.080 | 1.00 | 18.66 | D |
| ATOM | 8774 | C | VAL D | 681 | 121.699 | 0.134 | 36.196 | 1.00 | 21.12 | D |
| ATOM | 8775 | O | VAL D | 681 | 122.429 | 1.030 | 35.748 | 1.00 | 19.50 | D |
| ATOM | 8776 | N | SER D | 682 | 120.404 | 0.039 | 35.956 | 1.00 | 22.21 | D |
| ATOM | 8777 | CA | SER D | 682 | 119.698 | 0.991 | 35.147 | 1.00 | 21.87 | D |
| ATOM | 8778 | CB | SER D | 682 | 119.633 | 0.520 | 33.703 | 1.00 | 20.44 | D |
| ATOM | 8779 | OG | SER D | 682 | 118.589 | −0.410 | 33.525 | 1.00 | 21.95 | D |
| ATOM | 8780 | C | SER D | 682 | 118.331 | 0.950 | 35.783 | 1.00 | 23.11 | D |
| ATOM | 8781 | O | SER D | 682 | 118.048 | 0.053 | 36.582 | 1.00 | 22.65 | D |
| ATOM | 8782 | N | ASP D | 683 | 117.483 | 1.910 | 35.457 | 1.00 | 23.07 | D |
| ATOM | 8783 | CA | ASP D | 683 | 116.165 | 1.901 | 36.050 | 1.00 | 23.72 | D |
| ATOM | 8784 | CB | ASP D | 683 | 115.359 | 3.107 | 35.591 | 1.00 | 26.85 | D |
| ATOM | 8785 | CG | ASP D | 683 | 115.822 | 4.384 | 36.224 | 1.00 | 30.20 | D |
| ATOM | 8786 | OD1 | ASP D | 683 | 116.151 | 4.341 | 37.431 | 1.00 | 35.54 | D |
| ATOM | 8787 | OD2 | ASP D | 683 | 115.840 | 5.421 | 35.521 | 1.00 | 28.97 | D |
| ATOM | 8788 | C | ASP D | 683 | 115.381 | 0.631 | 35.728 | 1.00 | 22.90 | D |
| ATOM | 8789 | O | ASP D | 683 | 114.466 | 0.281 | 36.458 | 1.00 | 22.58 | D |
| ATOM | 8790 | N | ASN D | 684 | 115.717 | −0.072 | 34.653 | 1.00 | 21.68 | D |
| ATOM | 8791 | CA | ASN D | 684 | 114.937 | −1.257 | 34.335 | 1.00 | 21.96 | D |
| ATOM | 8792 | CB | ASN D | 684 | 114.568 | −1.291 | 32.842 | 1.00 | 20.91 | D |
| ATOM | 8793 | CG | ASN D | 684 | 113.639 | −0.138 | 32.417 | 1.00 | 22.88 | D |
| ATOM | 8794 | OD1 | ASN D | 684 | 112.685 | 0.229 | 33.132 | 1.00 | 20.08 | D |
| ATOM | 8795 | ND2 | ASN D | 684 | 113.907 | 0.423 | 31.231 | 1.00 | 21.27 | D |
| ATOM | 8796 | C | ASN D | 684 | 115.561 | −2.597 | 34.705 | 1.00 | 22.28 | D |
| ATOM | 8797 | O | ASN D | 684 | 114.840 | −3.557 | 34.983 | 1.00 | 21.62 | D |
| ATOM | 8798 | N | ILE D | 685 | 116.890 | −2.659 | 34.735 | 1.00 | 22.22 | D |
| ATOM | 8799 | CA | ILE D | 685 | 117.592 | −3.906 | 35.022 | 1.00 | 24.26 | D |
| ATOM | 8800 | CB | ILE D | 685 | 118.241 | −4.431 | 33.725 | 1.00 | 25.72 | D |
| ATOM | 8801 | CG2 | ILE D | 685 | 119.014 | −5.722 | 33.987 | 1.00 | 28.28 | D |
| ATOM | 8802 | CG1 | ILE D | 685 | 117.181 | −4.607 | 32.646 | 1.00 | 26.62 | D |
| ATOM | 8803 | CD1 | ILE D | 685 | 117.527 | −3.851 | 31.344 | 1.00 | 32.32 | D |
| ATOM | 8804 | C | ILE D | 685 | 118.701 | −3.830 | 36.081 | 1.00 | 25.44 | D |
| ATOM | 8805 | O | ILE D | 685 | 119.291 | −2.771 | 36.300 | 1.00 | 25.75 | D |
| ATOM | 8806 | N | LEU D | 686 | 118.989 | −4.964 | 36.717 | 1.00 | 25.66 | D |
| ATOM | 8807 | CA | LEU D | 686 | 120.069 | −5.041 | 37.689 | 1.00 | 26.90 | D |
| ATOM | 8808 | CB | LEU D | 686 | 119.530 | −5.089 | 39.127 | 1.00 | 26.08 | D |
| ATOM | 8809 | CG | LEU D | 686 | 120.472 | −5.572 | 40.262 | 1.00 | 23.99 | D |
| ATOM | 8810 | CD1 | LEU D | 686 | 121.709 | −4.699 | 40.345 | 1.00 | 23.40 | D |
| ATOM | 8811 | CD2 | LEU D | 686 | 119.740 | −5.567 | 41.585 | 1.00 | 21.28 | D |
| ATOM | 8812 | C | LEU D | 686 | 120.859 | −6.317 | 37.412 | 1.00 | 28.01 | D |
| ATOM | 8813 | O | LEU D | 686 | 120.275 | −7.397 | 37.340 | 1.00 | 28.42 | D |
| ATOM | 8814 | N | VAL D | 687 | 122.170 | −6.194 | 37.226 | 1.00 | 26.81 | D |
| ATOM | 8815 | CA | VAL D | 687 | 122.995 | −7.371 | 37.019 | 1.00 | 28.16 | D |
| ATOM | 8816 | CB | VAL D | 687 | 123.648 | −7.401 | 35.629 | 1.00 | 28.88 | D |
| ATOM | 8817 | CG1 | VAL D | 687 | 124.496 | −8.669 | 35.484 | 1.00 | 27.76 | D |
| ATOM | 8818 | CG2 | VAL D | 687 | 122.562 | −7.383 | 34.553 | 1.00 | 29.55 | D |
| ATOM | 8819 | C | VAL D | 687 | 124.064 | −7.347 | 38.098 | 1.00 | 29.32 | D |
| ATOM | 8820 | O | VAL D | 687 | 124.744 | −6.331 | 38.291 | 1.00 | 29.09 | D |
| ATOM | 8821 | N | SER D | 688 | 124.200 | −8.465 | 38.813 | 1.00 | 28.34 | D |
| ATOM | 8822 | CA | SER D | 688 | 125.166 | −8.570 | 39.910 | 1.00 | 27.42 | D |
| ATOM | 8823 | CB | SER D | 688 | 124.419 | −8.567 | 41.247 | 1.00 | 26.52 | D |
| ATOM | 8824 | OG | SER D | 688 | 123.505 | −9.651 | 41.304 | 1.00 | 25.52 | D |
| ATOM | 8825 | C | SER D | 688 | 125.990 | −9.853 | 39.808 | 1.00 | 27.67 | D |
| ATOM | 8826 | O | SER D | 688 | 125.518 | −10.870 | 39.304 | 1.00 | 28.15 | D |
| ATOM | 8827 | N | GLY D | 689 | 127.217 | −9.818 | 40.299 | 1.00 | 25.88 | D |
| ATOM | 8828 | CA | GLY D | 689 | 128.012 | −11.016 | 40.228 | 1.00 | 25.97 | D |
| ATOM | 8829 | C | GLY D | 689 | 128.901 | −11.218 | 41.422 | 1.00 | 27.10 | D |
| ATOM | 8830 | O | GLY D | 689 | 129.549 | −10.285 | 41.890 | 1.00 | 29.72 | D |
| ATOM | 8831 | N | SER D | 690 | 128.914 | −12.445 | 41.922 | 1.00 | 27.15 | D |
| ATOM | 8832 | CA | SER D | 690 | 129.752 | −12.819 | 43.042 | 1.00 | 27.45 | D |
| ATOM | 8833 | CB | SER D | 690 | 128.918 | −13.015 | 44.300 | 1.00 | 26.48 | D |
| ATOM | 8834 | OG | SER D | 690 | 127.975 | −14.044 | 44.099 | 1.00 | 25.71 | D |
| ATOM | 8835 | C | SER D | 690 | 130.376 | −14.145 | 42.630 | 1.00 | 28.76 | D |
| ATOM | 8836 | O | SER D | 690 | 130.435 | −14.457 | 41.444 | 1.00 | 28.74 | D |
| ATOM | 8837 | N | GLU D | 691 | 130.829 | −14.925 | 43.609 | 1.00 | 29.69 | D |
| ATOM | 8838 | CA | GLU D | 691 | 131.446 | −16.210 | 43.320 | 1.00 | 29.80 | D |
| ATOM | 8839 | CB | GLU D | 691 | 132.241 | −16.711 | 44.532 | 1.00 | 30.89 | D |
| ATOM | 8840 | CG | GLU D | 691 | 132.740 | −18.153 | 44.426 | 1.00 | 34.76 | D |
| ATOM | 8841 | CD | GLU D | 691 | 133.806 | −18.516 | 45.469 | 1.00 | 37.18 | D |
| ATOM | 8842 | OE1 | GLU D | 691 | 134.895 | −17.901 | 45.448 | 1.00 | 39.22 | D |
| ATOM | 8843 | OE2 | GLU D | 691 | 133.564 | −19.420 | 46.305 | 1.00 | 38.14 | D |
| ATOM | 8844 | C | GLD D | 691 | 130.409 | −17.239 | 42.888 | 1.00 | 29.94 | D |
| ATOM | 8845 | O | GLU D | 691 | 129.441 | −17.507 | 43.596 | 1.00 | 28.81 | D |
| ATOM | 8846 | N | ASN D | 692 | 130.621 | −17.784 | 41.693 | 1.00 | 30.68 | D |
| ATOM | 8847 | CA | ASN D | 692 | 129.756 | −18.800 | 41.122 | 1.00 | 30.63 | D |
| ATOM | 8848 | CB | ASN D | 692 | 129.854 | −20.070 | 41.975 | 1.00 | 32.82 | D |
| ATOM | 8849 | CG | ASN D | 692 | 131.019 | −20.951 | 41.562 | 1.00 | 34.81 | D |
| ATOM | 8850 | OD1 | ASN D | 692 | 130.832 | −21.908 | 40.812 | 1.00 | 39.00 | D |
| ATOM | 8851 | ND2 | ASN D | 692 | 132.231 | −20.614 | 42.016 | 1.00 | 34.40 | D |
| ATOM | 8852 | C | ASN D | 692 | 128.313 | −18.370 | 40.941 | 1.00 | 30.73 | D |
| ATOM | 8853 | O | ASN D | 692 | 127.395 | −19.187 | 40.996 | 1.00 | 31.82 | D |
| ATOM | 8854 | N | GLN D | 693 | 128.113 | −17.081 | 40.711 | 1.00 | 30.83 | D |
| ATOM | 8855 | CA | GLN D | 693 | 126.774 | −16.564 | 40.496 | 1.00 | 30.85 | D |
| ATOM | 8856 | CB | GLN D | 693 | 126.127 | −16.215 | 41.837 | 1.00 | 32.95 | D |
| ATOM | 8857 | CG | GLN D | 693 | 125.494 | −17.398 | 42.562 | 1.00 | 36.48 | D |
| ATOM | 8858 | CD | CLN D | 693 | 124.636 | −16.949 | 43.741 | 1.00 | 39.48 | D |
| ATOM | 8859 | OE1 | GLN D | 693 | 123.650 | −16.222 | 43.571 | 1.00 | 40.51 | D |
| ATOM | 8860 | NE2 | GLN D | 693 | 125.014 | −17.371 | 44.941 | 1.00 | 40.67 | D |
| ATOM | 8861 | C | GLN D | 693 | 126.771 | −15.350 | 39.560 | 1.00 | 30.20 | D |
| ATOM | 8862 | O | GLN D | 693 | 127.694 | −14.533 | 39.586 | 1.00 | 28.95 | D |
| ATOM | 8863 | N | PHE D | 694 | 125.729 | −15.268 | 38.728 | 1.00 | 28.92 | D |
| ATOM | 8864 | CA | PHE D | 694 | 125.534 | −14.189 | 37.758 | 1.00 | 27.46 | D |
| ATOM | 8865 | CB | PHE D | 694 | 126.183 | −14.549 | 36.404 | 1.00 | 26.85 | D |
| ATOM | 8866 | CG | PHE D | 694 | 126.212 | −13.402 | 35.413 | 1.00 | 27.99 | D |
| ATOM | 8867 | CD1 | PHE D | 694 | 127.027 | −12.290 | 35.626 | 1.00 | 28.31 | D |
| ATOM | 8868 | CD2 | PHE D | 694 | 125.387 | −13.411 | 34.291 | 1.00 | 28.25 | D |
| ATOM | 8869 | CE1 | PHE D | 694 | 127.011 | −11.201 | 34.734 | 1.00 | 25.87 | D |
| ATOM | 8870 | CE2 | PHE D | 694 | 125.365 | −12.329 | 33.397 | 1.00 | 26.35 | D |
| ATOM | 8871 | CZ | PHE D | 694 | 126.175 | −11.226 | 33.620 | 1.00 | 24.43 | D |
| ATOM | 8872 | C | PHE D | 694 | 124.018 | −14.020 | 37.600 | 1.00 | 26.65 | D |
| ATOM | 8873 | O | PHE D | 694 | 123.360 | −14.804 | 36.923 | 1.00 | 27.90 | D |
| ATOM | 8874 | N | ASN D | 695 | 123.462 | −12.997 | 38.235 | 1.00 | 25.11 | D |
| ATOM | 8875 | CA | ASN D | 695 | 122.033 | −12.784 | 38.169 | 1.00 | 22.66 | D |
| ATOM | 8876 | CE | ASN D | 695 | 121.462 | −12.735 | 39.570 | 1.00 | 21.74 | D |
| ATOM | 8877 | CG | ASN D | 695 | 121.802 | −13.948 | 40.351 | 1.00 | 22.53 | D |
| ATOM | 8878 | OD1 | ASN D | 695 | 122.469 | −13.863 | 41.381 | 1.00 | 25.44 | D |
| ATOM | 8879 | ND2 | ASN D | 695 | 121.369 | −15.102 | 39.866 | 1.00 | 21.60 | D |
| ATOM | 8880 | C | ASN D | 695 | 121.586 | −11.557 | 37.431 | 1.00 | 22.56 | D |
| ATOM | 8881 | O | ASN D | 695 | 122.283 | −10.537 | 37.402 | 1.00 | 24.25 | D |
| ATOM | 8882 | N | ILE D | 696 | 120.407 | −11.675 | 36.828 | 1.00 | 20.27 | D |
| ATOM | 8883 | CA | ILE D | 696 | 119.785 | −10.570 | 36.112 | 1.00 | 18.17 | D |
| ATOM | 8884 | CB | ILE D | 696 | 119.741 | −10.848 | 34.598 | 1.00 | 13.96 | D |
| ATOM | 8885 | CG2 | ILE D | 696 | 119.173 | −9.631 | 33.874 | 1.00 | 12.40 | D |
| ATOM | 8886 | CG1 | ILE D | 696 | 121.165 | −11.096 | 34.095 | 1.00 | 11.96 | D |
| ATOM | 8887 | CD1 | ILE D | 696 | 121.278 | −11.411 | 32.627 | 1.00 | 10.15 | D |
| ATOM | 8888 | C | ILE D | 696 | 118.375 | −10.387 | 36.708 | 1.00 | 19.43 | D |
| ATOM | 8889 | O | ILE D | 696 | 117.558 | −11.307 | 36.714 | 1.00 | 19.49 | D |
| ATOM | 8890 | N | TYR D | 697 | 118.130 | −9.194 | 37.236 | 1.00 | 18.77 | D |
| ATOM | 8891 | CA | TYR D | 697 | 116.878 | −8.867 | 37.881 | 1.00 | 17.94 | D |
| ATOM | 8892 | CB | TYR D | 697 | 117.167 | −8.295 | 39.280 | 1.00 | 19.60 | D |
| ATOM | 8893 | CG | TYR D | 697 | 118.014 | −9.185 | 40.171 | 1.00 | 19.57 | D |
| ATOM | 8894 | CD1 | TYR D | 697 | 117.428 | −10.186 | 40.962 | 1.00 | 20.99 | D |
| ATOM | 8895 | CE1 | TYR D | 697 | 118.204 | −11.012 | 41.789 | 1.00 | 18.57 | D |
| ATOM | 8896 | CD2 | TYR D | 697 | 119.397 | −9.031 | 40.223 | 1.00 | 18.67 | D |
| ATOM | 8897 | CE2 | TYR D | 697 | 120.179 | −9.840 | 41.035 | 1.00 | 19.21 | D |
| ATOM | 8898 | CZ | TYR D | 697 | 119.581 | −10.832 | 41.820 | 1.00 | 20.43 | D |
| ATOM | 8899 | OH | TYR D | 697 | 120.372 | −11.634 | 42.627 | 1.00 | 20.44 | D |
| ATOM | 8900 | C | TYR D | 697 | 116.059 | −7.843 | 37.104 | 1.00 | 18.57 | D |
| ATOM | 8901 | O | TYR D | 697 | 116.604 | −6.934 | 36.456 | 1.00 | 18.53 | D |
| ATOM | 8902 | N | ASN D | 698 | 114.746 | −7.975 | 37.208 | 1.00 | 16.25 | D |
| ATOM | 8903 | CA | ASN D | 698 | 113.826 | −7.049 | 36.575 | 1.00 | 16.13 | D |
| ATOM | 8904 | CB | ASN D | 698 | 112.594 | −7.832 | 36.111 | 1.00 | 16.70 | D |
| ATOM | 8905 | CG | ASN D | 698 | 111.516 | −6.939 | 35.508 | 1.00 | 18.93 | D |
| ATOM | 8906 | OD1 | ASN D | 698 | 111.253 | −5.822 | 36.004 | 1.00 | 16.72 | D |
| ATOM | 8907 | ND2 | ASN D | 698 | 110.873 | −7.428 | 34.443 | 1.00 | 16.48 | D |
| ATOM | 8908 | C | ASN D | 698 | 113.441 | −6.032 | 37.682 | 1.00 | 15.83 | D |
| ATOM | 8909 | O | ASN D | 698 | 112.568 | −6.301 | 38.499 | 1.00 | 14.84 | D |
| ATOM | 8910 | N | LEU D | 699 | 114.078 | −4.874 | 37.735 | 1.00 | 14.22 | D |
| ATOM | 8911 | CA | LEU D | 699 | 113.725 | −3.948 | 38.796 | 1.00 | 15.47 | D |
| ATOM | 8912 | CB | LEU D | 699 | 114.549 | −2.673 | 38.704 | 1.00 | 11.67 | D |
| ATOM | 8913 | CG | LEU D | 699 | 115.818 | −2.596 | 39.541 | 1.00 | 12.58 | D |
| ATOM | 8914 | CD | LEU D | 699 | 116.357 | −4.000 | 39.957 | 1.00 | 9.17 | D |
| ATOM | 8915 | CD2 | LEU D | 699 | 116.822 | −1.809 | 38.724 | 1.00 | 9.81 | D |
| ATOM | 8916 | C | LEU D | 699 | 112.260 | −3.560 | 38.883 | 1.00 | 18.10 | D |
| ATOM | 8917 | O | LEU D | 699 | 111.836 | −3.053 | 39.914 | 1.00 | 21.11 | D |
| ATOM | 8918 | N | ARG D | 700 | 111.479 | −3.765 | 37.826 | 1.00 | 19.06 | D |
| ATOM | 8919 | CA | ARG D | 700 | 110.063 | −3.382 | 37.889 | 1.00 | 18.41 | D |
| ATOM | 8920 | CB | ARG D | 700 | 109.474 | −3.190 | 36.481 | 1.00 | 16.13 | D |
| ATOM | 8921 | CG | ARG D | 700 | 110.078 | −2.053 | 35.660 | 1.00 | 18.32 | D |
| ATOM | 8922 | CD | ARG D | 700 | 109.605 | −2.100 | 34.188 | 1.00 | 19.47 | D |
| ATOM | 8923 | NE | ARG D | 700 | 110.340 | −1.195 | 33.295 | 1.00 | 19.60 | D |
| ATOM | 8924 | CZ | ARG D | 700 | 109.881 | −0.738 | 32.127 | 1.00 | 20.84 | D |
| ATOM | 8925 | NH1 | ARG D | 700 | 108.678 | −1.085 | 31.693 | 1.00 | 19.16 | D |
| ATOM | 8926 | NH2 | ARG D | 700 | 110.633 | 0.057 | 31.378 | 1.00 | 20.52 | D |
| ATOM | 8927 | C | ARG D | 700 | 109.211 | −4.411 | 38.652 | 1.00 | 19.19 | D |
| ATOM | 8928 | O | ARG D | 700 | 108.440 | −4.069 | 39.546 | 1.00 | 20.01 | D |
| ATOM | 8929 | N | SER D | 701 | 109.344 | −5.673 | 38.286 | 1.00 | 18.61 | D |
| ATOM | 8930 | CA | SER D | 701 | 108.580 | −6.719 | 38.919 | 1.00 | 18.99 | D |
| ATOM | 8931 | CB | SER D | 701 | 108.403 | −7.907 | 37.960 | 1.00 | 17.26 | D |
| ATOM | 8932 | OG | SER D | 701 | 109.627 | −8.299 | 37.350 | 1.00 | 16.41 | D |
| ATOM | 8933 | C | SER D | 701 | 109.317 | −7.179 | 40.154 | 1.00 | 21.34 | D |
| ATOM | 8934 | O | SER D | 701 | 108.758 | −7.881 | 40.983 | 1.00 | 21.67 | D |
| ATOM | 8935 | N | GLY D | 702 | 110.577 | −6.782 | 40.277 | 1.00 | 23.17 | D |
| ATOM | 8936 | CA | GLY D | 702 | 111.367 | −7.189 | 41.420 | 1.00 | 25.46 | D |
| ATOM | 8937 | C | GLY D | 702 | 111.916 | −8.601 | 41.338 | 1.00 | 26.82 | D |
| ATOM | 8938 | O | GLY D | 702 | 112.676 | −9.008 | 42.208 | 1.00 | 28.70 | D |
| ATOM | 8939 | N | LYS D | 703 | 111.580 | −9.346 | 40.290 | 1.00 | 28.39 | D |
| ATOM | 8940 | CA | LYS D | 703 | 112.040 | −10.734 | 40.187 | 1.00 | 29.97 | D |
| ATOM | 8941 | CE | LYS D | 703 | 110.926 | −11.616 | 39.609 | 1.00 | 30.86 | D |
| ATOM | 8942 | CG | LYS D | 703 | 109.566 | −11.294 | 40.211 | 1.00 | 34.24 | D |
| ATOM | 8943 | CD | LYS D | 703 | 108.553 | −12.393 | 40.031 | 1.00 | 36.04 | D |
| ATOM | 8944 | CE | LYS D | 703 | 108.971 | −13.657 | 40.786 | 1.00 | 38.38 | D |
| ATOM | 8945 | NZ | LYS D | 703 | 107.900 | −14.711 | 40.780 | 1.00 | 39.62 | D |
| ATOM | 8946 | C | LYS D | 703 | 113.319 | −11.022 | 39.428 | 1.00 | 30.16 | D |
| ATOM | 8947 | O | LYS D | 703 | 113.822 | −10.202 | 38.665 | 1.00 | 33.16 | D |
| ATOM | 8948 | N | LEU D | 704 | 113.824 | −12.227 | 39.655 | 1.00 | 31.22 | D |
| ATOM | 8949 | CA | LEU D | 704 | 115.037 | −12.717 | 39.024 | 1.00 | 31.13 | D |
| ATOM | 8950 | CE | LEU D | 704 | 115.655 | −13.799 | 39.894 | 1.00 | 28.59 | D |
| ATOM | 8951 | CG | LEU D | 704 | 116.959 | −14.425 | 39.433 | 1.00 | 29.39 | D |
| ATOM | 8952 | CD1 | LEU D | 704 | 118.092 | −13.385 | 39.446 | 1.00 | 31.25 | D |
| ATOM | 8953 | CD2 | LEU D | 704 | 117.298 | −15.548 | 40.368 | 1.00 | 27.62 | D |
| ATOM | 8954 | C | LEU D | 704 | 114.663 | −13.302 | 37.668 | 1.00 | 32.42 | D |
| ATOM | 8955 | O | LEU D | 704 | 113.791 | −14.167 | 37.585 | 1.00 | 34.11 | D |
| ATOM | 8956 | N | VAL D | 705 | 115.308 | −12.827 | 36.607 | 1.00 | 32.96 | D |
| ATOM | 8957 | CA | VAL D | 705 | 115.014 | −13.329 | 35.275 | 1.00 | 33.27 | D |
| ATOM | 8958 | CB | VAL D | 705 | 115.052 | −12.240 | 34.214 | 1.00 | 31.84 | D |
| ATOM | 8959 | CG1 | VAL D | 705 | 114.710 | −12.853 | 32.866 | 1.00 | 33.49 | D |
| ATOM | 8960 | CG2 | VAL D | 705 | 114.079 | −11.142 | 34.544 | 1.00 | 30.65 | D |
| ATOM | 8961 | C | VAL D | 705 | 116.011 | −14.389 | 34.841 | 1.00 | 35.05 | D |
| ATOM | 8962 | O | VAL D | 705 | 115.632 | −15.501 | 34.504 | 1.00 | 37.11 | D |
| ATOM | 8963 | N | HIS D | 706 | 117.291 | −14.041 | 34.827 | 1.00 | 36.45 | D |
| ATOM | 8964 | CA | HIS D | 706 | 118.316 | −14.991 | 34.426 | 1.00 | 36.74 | D |
| ATOM | 8965 | CB | HIS D | 706 | 119.007 | −14.539 | 33.137 | 1.00 | 37.71 | D |
| ATOM | 8966 | CG | HIS D | 706 | 118.064 | −14.142 | 32.042 | 1.00 | 40.20 | D |
| ATOM | 8967 | CD2 | HIS D | 706 | 117.901 | −12.964 | 31.392 | 1.00 | 41.01 | D |
| ATOM | 8968 | ND1 | HIS D | 706 | 117.172 | −15.022 | 31.468 | 1.00 | 40.45 | D |
| ATOM | 8969 | CE1 | HIS D | 706 | 116.504 | −14.404 | 30.509 | 1.00 | 41.40 | D |
| ATOM | 8970 | NE2 | HIS D | 706 | 116.927 | −13.155 | 30.441 | 1.00 | 41.00 | D |
| ATOM | 8971 | C | HIS D | 706 | 119.361 | −15.155 | 35.527 | 1.00 | 37.89 | D |
| ATOM | 8972 | O | HIS D | 706 | 119.869 | −14.174 | 36.096 | 1.00 | 38.65 | D |
| ATOM | 8973 | N | ALA D | 707 | 119.686 | −16.414 | 35.805 | 1.00 | 37.80 | D |
| ATOM | 8974 | CA | ALA D | 707 | 120.660 | −16.757 | 36.828 | 1.00 | 36.66 | D |
| ATOM | 8975 | CB | ALA D | 707 | 119.935 | −17.223 | 38.075 | 1.00 | 33.53 | D |
| ATOM | 8976 | C | ALA D | 707 | 121.651 | −17.828 | 36.367 | 1.00 | 36.44 | D |
| ATOM | 8977 | O | ALA D | 707 | 122.608 | −18.138 | 37.079 | 1.00 | 36.66 | D |
| ATOM | 8978 | N | ASN D | 708 | 121.446 | −18.382 | 35.177 | 1.00 | 36.37 | D |
| ATOM | 8979 | CA | ASN D | 708 | 122.347 | −19.431 | 34.710 | 1.00 | 37.18 | D |
| ATOM | 8980 | CB | ASN D | 708 | 121.553 | −20.739 | 34.521 | 1.00 | 37.73 | D |
| ATOM | 8981 | CG | ASN D | 708 | 120.893 | −21.212 | 35.821 | 1.00 | 38.57 | D |
| ATOM | 8982 | OD1 | ASN D | 708 | 119.667 | −21.227 | 35.947 | 1.00 | 38.35 | D |
| ATOM | 8983 | ND2 | ASN D | 708 | 121.712 | −21.588 | 36.794 | 1.00 | 37.37 | D |
| ATOM | 8984 | C | ASN D | 708 | 123.137 | −19.084 | 33.443 | 1.00 | 36.59 | D |
| ATOM | 8985 | O | ASN D | 708 | 123.856 | −19.927 | 32.907 | 1.00 | 35.29 | D |
| ATOM | 8986 | N | ILE D | 709 | 123.019 | −17.839 | 32.986 | 1.00 | 35.82 | D |
| ATOM | 8987 | CA | ILE D | 709 | 123.709 | −17.402 | 31.781 | 1.00 | 34.43 | D |
| ATOM | 8988 | CB | ILE D | 709 | 123.349 | −15.928 | 31.408 | 1.00 | 32.34 | D |
| ATOM | 8989 | CG2 | ILE D | 709 | 124.151 | −15.460 | 30.221 | 1.00 | 30.43 | D |
| ATOM | 8990 | CG1 | ILE D | 709 | 121.875 | −15.836 | 31.017 | 1.00 | 30.65 | D |
| ATOM | 8991 | CD1 | ILE D | 709 | 121.444 | −14.459 | 30.565 | 1.00 | 28.79 | D |
| ATOM | 8992 | C | ILE D | 709 | 125.216 | −17.552 | 31.873 | 1.00 | 34.88 | D |
| ATOM | 8993 | O | ILE D | 709 | 125.863 | −17.878 | 30.882 | 1.00 | 36.86 | D |
| ATOM | 8994 | N | LEU D | 710 | 125.785 | −17.336 | 33.051 | 1.00 | 34.63 | D |
| ATOM | 8995 | CA | LEU D | 710 | 127.232 | −17.454 | 33.186 | 1.00 | 32.46 | D |
| ATOM | 8996 | CB | LEU D | 710 | 127.837 | −16.058 | 33.402 | 1.00 | 28.39 | D |
| ATOM | 8997 | CG | LEU D | 710 | 128.450 | −15.306 | 32.208 | 1.00 | 25.87 | D |
| ATOM | 8998 | CD1 | LEU D | 710 | 127.798 | −15.707 | 30.894 | 1.00 | 24.33 | D |
| ATOM | 8999 | CD2 | LEU D | 710 | 128.331 | −13.810 | 32.443 | 1.00 | 23.04 | D |
| ATOM | 9000 | C | LEU D | 710 | 127.683 | −18.434 | 34.269 | 1.00 | 33.52 | D |
| ATOM | 9001 | O | LEU D | 710 | 128.730 | −18.246 | 34.882 | 1.00 | 34.43 | D |
| ATOM | 9002 | N | LYS D | 711 | 126.902 | −19.494 | 34.480 | 1.00 | 34.36 | D |
| ATOM | 9003 | CA | LYS D | 711 | 127.230 | −20.518 | 35.481 | 1.00 | 35.44 | D |
| ATOM | 9004 | CB | LYS D | 711 | 126.238 | −21.670 | 35.389 | 1.00 | 34.96 | D |
| ATOM | 9005 | CG | LYS D | 711 | 125.872 | −22.006 | 33.980 | 1.00 | 36.79 | D |
| ATOM | 9006 | CD | LYS D | 711 | 124.620 | −22.834 | 33.940 | 1.00 | 38.39 | D |
| ATOM | 9007 | CE | LYS D | 711 | 124.936 | −24.285 | 34.192 | 1.00 | 40.21 | D |
| ATOM | 9008 | NZ | LYS D | 711 | 125.841 | −24.794 | 33.104 | 1.00 | 42.27 | D |
| ATOM | 9009 | C | LYS D | 711 | 128.645 | −21.068 | 35.350 | 1.00 | 36.53 | D |
| ATOM | 9010 | O | LYS D | 711 | 129.284 | −21.427 | 36.341 | 1.00 | 36.63 | D |
| ATOM | 9011 | N | ASP D | 712 | 129.130 | −21.126 | 34.121 | 1.00 | 37.64 | D |
| ATOM | 9012 | CA | ASP D | 712 | 130.457 | −21.629 | 33.836 | 1.00 | 39.74 | D |
| ATOM | 9013 | CB | ASP D | 712 | 130.688 | −21.555 | 32.336 | 1.00 | 44.17 | D |
| ATOM | 9014 | CG | ASP D | 712 | 129.490 | −22.032 | 31.558 | 1.00 | 49.91 | D |
| ATOM | 9015 | OD1 | ASP D | 712 | 129.338 | −23.276 | 31.408 | 1.00 | 53.21 | D |
| ATOM | 9016 | OD2 | ASP D | 712 | 128.684 | −21.163 | 31.122 | 1.00 | 51.40 | D |
| ATOM | 9017 | C | ASP D | 712 | 131.548 | −20.856 | 34.551 | 1.00 | 39.02 | D |
| ATOM | 9018 | O | ASP D | 712 | 132.528 | −21.439 | 34.992 | 1.00 | 39.79 | D |
| ATOM | 9019 | N | ALA D | 713 | 131.398 | −19.542 | 34.647 | 1.00 | 37.44 | D |
| ATOM | 9020 | CA | ALA D | 713 | 132.427 | −18.740 | 35.295 | 1.00 | 37.31 | D |
| ATOM | 9021 | CB | ALA D | 713 | 132.131 | −17.254 | 35.119 | 1.00 | 37.40 | D |
| ATOM | 9022 | C | ALA D | 713 | 132.603 | −19.055 | 36.775 | 1.00 | 36.89 | D |
| ATOM | 9023 | O | ALA D | 713 | 131.693 | −19.535 | 37.437 | 1.00 | 37.66 | D |
| ATOM | 9024 | N | ASP D | 714 | 133.785 | −18.782 | 37.296 | 1.00 | 35.36 | D |
| ATOM | 9025 | CA | ASP D | 714 | 134.018 | −19.033 | 38.693 | 1.00 | 34.87 | D |
| ATOM | 9026 | CB | ASP D | 714 | 135.384 | −19.678 | 38.893 | 1.00 | 33.94 | D |
| ATOM | 9027 | CG | ASP D | 714 | 135.484 | −21.038 | 38.244 | 1.00 | 32.23 | D |
| ATOM | 9028 | OD1 | ASP D | 714 | 134.599 | −21.877 | 38.477 | 1.00 | 32.51 | D |
| ATOM | 9029 | OD2 | ASP D | 714 | 136.456 | −21.279 | 37.505 | 1.00 | 34.54 | D |
| ATOM | 9030 | C | ASP D | 714 | 133.946 | −17.705 | 39.433 | 1.00 | 35.72 | D |
| ATOM | 9031 | O | ASP D | 714 | 133.610 | −17.651 | 40.616 | 1.00 | 35.20 | D |
| ATOM | 9032 | N | GLN D | 715 | 134.238 | −16.629 | 38.714 | 1.00 | 35.93 | D |
| ATOM | 9033 | CA | GLN D | 715 | 134.233 | −15.293 | 39.284 | 1.00 | 36.18 | D |
| ATOM | 9034 | CE | GLN D | 715 | 135.648 | −14.938 | 39.750 | 1.00 | 37.10 | D |
| ATOM | 9035 | CG | GLN D | 715 | 136.260 | −15.951 | 40.716 | 1.00 | 39.05 | D |
| ATOM | 9036 | CD | GLN D | 715 | 135.897 | −15.672 | 42.165 | 1.00 | 40.12 | D |
| ATOM | 9037 | OE1 | GLN D | 715 | 134.751 | −15.380 | 42.472 | 1.00 | 42.68 | D |
| ATOM | 9038 | NE2 | GLN D | 715 | 136.874 | −15.763 | 43.057 | 1.00 | 39.97 | D |
| ATOM | 9039 | C | GLN D | 715 | 133.756 | −14.264 | 38.254 | 1.00 | 35.81 | D |
| ATOM | 9040 | O | GLN D | 715 | 133.993 | −14.407 | 37.054 | 1.00 | 35.76 | D |
| ATOM | 9041 | N | ILE D | 716 | 133.073 | −13.232 | 38.736 | 1.00 | 34.56 | D |
| ATOM | 9042 | CA | ILE D | 716 | 132.582 | −12.149 | 37.888 | 1.00 | 32.51 | D |
| ATOM | 9043 | CB | ILE D | 716 | 131.061 | −11.982 | 38.032 | 1.00 | 31.79 | D |
| ATOM | 9044 | CG2 | ILE D | 716 | 130.547 | −11.006 | 36.996 | 1.00 | 30.49 | D |
| ATOM | 9045 | CG1 | ILE D | 716 | 130.363 | −13.336 | 37.890 | 1.00 | 33.24 | D |
| ATOM | 9046 | CD1 | ILE D | 716 | 130.638 | −14.060 | 36.579 | 1.00 | 34.30 | D |
| ATOM | 9047 | C | ILE D | 716 | 133.285 | −10.863 | 38.375 | 1.00 | 32.59 | D |
| ATOM | 9048 | O | ILE D | 716 | 132.797 | −10.160 | 39.264 | 1.00 | 33.77 | D |
| ATOM | 9049 | N | TRP D | 717 | 134.444 | −10.580 | 37.795 | 1.00 | 30.84 | D |
| ATOM | 9050 | CA | TRP D | 717 | 135.252 | −9.429 | 38.162 | 1.00 | 29.11 | D |
| ATOM | 9051 | CE | TRP D | 717 | 136.558 | −9.454 | 37.379 | 1.00 | 32.71 | D |
| ATOM | 9052 | CG | TRP D | 717 | 137.348 | −10.687 | 37.596 | 1.00 | 35.43 | D |
| ATOM | 9053 | CD2 | TRP D | 717 | 137.669 | −11.278 | 38.855 | 1.00 | 35.53 | D |
| ATOM | 9054 | CE2 | TRP D | 717 | 138.462 | −12.416 | 38.589 | 1.00 | 36.83 | D |
| ATOM | 9055 | CE3 | TRP D | 717 | 137.364 | −10.960 | 40.185 | 1.00 | 35.83 | D |
| ATOM | 9056 | CD1 | TRP D | 717 | 137.940 | −11.464 | 36.640 | 1.00 | 36.35 | D |
| ATOM | 9057 | NE1 | TRP D | 717 | 138.612 | −12.504 | 37.230 | 1.00 | 37.07 | D |
| ATOM | 9058 | CZ2 | TRP D | 717 | 138.956 | −13.237 | 39.605 | 1.00 | 38.32 | D |
| ATOM | 9059 | CZ3 | TRP D | 717 | 137.855 | −11.780 | 41.193 | 1.00 | 37.12 | D |
| ATOM | 9060 | CH2 | TRP D | 717 | 138.641 | −12.904 | 40.896 | 1.00 | 37.38 | D |
| ATOM | 9061 | C | TRP D | 717 | 134.583 | −8.102 | 37.930 | 1.00 | 26.79 | D |
| ATOM | 9062 | O | TRP D | 717 | 134.507 | −7.262 | 38.825 | 1.00 | 24.62 | D |
| ATOM | 9063 | N | SER D | 718 | 134.110 | −7.899 | 36.713 | 1.00 | 24.96 | D |
| ATOM | 9064 | CA | SER D | 718 | 133.473 | −6.639 | 36.403 | 1.00 | 23.33 | D |
| ATOM | 9065 | CB | SER D | 718 | 134.499 | −5.674 | 35.830 | 1.00 | 21.39 | D |
| ATOM | 9066 | OG | SER D | 718 | 133.898 | −4.420 | 35.620 | 1.00 | 24.14 | D |
| ATOM | 9067 | C | SER D | 718 | 132.347 | −6.825 | 35.421 | 1.00 | 22.63 | D |
| ATOM | 9068 | O | SER D | 718 | 132.481 | −7.549 | 34.443 | 1.00 | 25.60 | D |
| ATOM | 9069 | N | VAL D | 719 | 131.228 | −6.175 | 35.679 | 1.00 | 21.31 | D |
| ATOM | 9070 | CA | VAL D | 719 | 130.095 | −6.286 | 34.784 | 1.00 | 23.17 | D |
| ATOM | 9071 | CB | VAL D | 719 | 128.900 | −6.926 | 35.517 | 1.00 | 23.18 | D |
| ATOM | 9072 | CG1 | VAL D | 719 | 128.244 | −5.916 | 36.483 | 1.00 | 23.48 | D |
| ATOM | 9073 | CG2 | VAL D | 719 | 127.915 | −7.427 | 34.517 | 1.00 | 24.77 | D |
| ATOM | 9074 | C | VAL D | 719 | 129.703 | −4.887 | 34.255 | 1.00 | 24.78 | D |
| ATOM | 9075 | O | VAL D | 719 | 130.024 | −3.864 | 34.872 | 1.00 | 25.63 | D |
| ATOM | 9076 | N | ASN D | 720 | 129.005 | −4.835 | 33.125 | 1.00 | 25.17 | D |
| ATOM | 9077 | CA | ASN D | 720 | 128.604 | −3.547 | 32.543 | 1.00 | 25.66 | D |
| ATOM | 9078 | CB | ASN D | 720 | 129.844 | −2.787 | 31.999 | 1.00 | 25.58 | D |
| ATOM | 9079 | CG | ASN D | 720 | 129.627 | −1.256 | 31.853 | 1.00 | 26.08 | D |
| ATOM | 9080 | OD1 | ASN D | 720 | 128.697 | −0.790 | 31.193 | 1.00 | 24.37 | D |
| ATOM | 9081 | ND2 | ASN D | 720 | 130.527 | −0.484 | 32.456 | 1.00 | 24.14 | D |
| ATOM | 9082 | C | ASN D | 720 | 127.665 | −3.882 | 31.397 | 1.00 | 25.69 | D |
| ATOM | 9083 | O | ASN D | 720 | 127.961 | −4.770 | 30.601 | 1.00 | 24.75 | D |
| ATOM | 9084 | N | PHE D | 721 | 126.524 | −3.209 | 31.324 | 1.00 | 25.70 | D |
| ATOM | 9085 | CA | PHE D | 721 | 125.610 | −3.473 | 30.226 | 1.00 | 25.92 | D |
| ATOM | 9086 | CB | PHE D | 721 | 124.486 | −4.426 | 30.663 | 1.00 | 24.85 | D |
| ATOM | 9087 | CG | PHE D | 721 | 123.483 | −3.803 | 31.566 | 1.00 | 23.85 | D |
| ATOM | 9088 | CD1 | PHE D | 721 | 122.441 | −3.052 | 31.043 | 1.00 | 22.92 | D |
| ATOM | 9089 | CD2 | PHE D | 721 | 123.588 | −3.938 | 32.941 | 1.00 | 22.57 | D |
| ATOM | 9090 | CE1 | PHE D | 721 | 121.525 | −2.442 | 31.877 | 1.00 | 21.91 | D |
| ATOM | 9091 | CE2 | PHE D | 721 | 122.675 | −3.328 | 33.777 | 1.00 | 22.55 | D |
| ATOM | 9092 | CZ | PHE D | 721 | 121.640 | −2.579 | 33.243 | 1.00 | 21.48 | D |
| ATOM | 9093 | C | PHE D | 721 | 125.076 | −2.145 | 29.706 | 1.00 | 26.21 | D |
| ATOM | 9094 | O | PHE D | 721 | 125.149 | −1.120 | 30.402 | 1.00 | 25.17 | D |
| ATOM | 9095 | N | LYS D | 722 | 124.564 | −2.166 | 28.473 | 1.00 | 26.41 | D |
| ATOM | 9096 | CA | LYS D | 722 | 124.046 | −0.966 | 27.801 | 1.00 | 25.77 | D |
| ATOM | 9097 | CB | LYS D | 722 | 125.230 | −0.140 | 27.287 | 1.00 | 25.66 | D |
| ATOM | 9098 | CG | LYS D | 722 | 125.220 | 1.340 | 27.627 | 1.00 | 26.55 | D |
| ATOM | 9099 | CD | LYS D | 722 | 124.193 | 2.106 | 26.828 | 1.00 | 28.07 | D |
| ATOM | 9100 | CE | LYS D | 722 | 124.609 | 3.586 | 26.571 | 1.00 | 31.04 | D |
| ATOM | 9101 | NZ | LYS D | 722 | 125.623 | 3.789 | 25.462 | 1.00 | 31.59 | D |
| ATOM | 9102 | C | LYS D | 722 | 123.152 | −1.376 | 26.621 | 1.00 | 25.54 | D |
| ATOM | 9103 | O | LYS D | 722 | 123.556 | −2.161 | 25.765 | 1.00 | 24.73 | D |
| ATOM | 9104 | N | GLY D | 723 | 121.936 | −0.851 | 26.580 | 1.00 | 25.69 | D |
| ATOM | 9105 | CA | GLY D | 723 | 121.044 | −1.201 | 25.497 | 1.00 | 25.26 | D |
| ATOM | 9106 | C | GLY D | 723 | 120.804 | −2.694 | 25.431 | 1.00 | 27.03 | D |
| ATOM | 9107 | O | GLY D | 723 | 120.226 | −3.287 | 26.342 | 1.00 | 28.21 | D |
| ATOM | 9108 | N | LYS D | 724 | 121.262 | −3.313 | 24.350 | 1.00 | 28.66 | D |
| ATOM | 9109 | CA | LYS D | 724 | 121.087 | −4.752 | 24.144 | 1.00 | 29.79 | D |
| ATOM | 9110 | CB | LYS D | 724 | 120.590 | −4.978 | 22.732 | 1.00 | 30.45 | D |
| ATOM | 9111 | CG | LYS D | 724 | 121.344 | −4.146 | 21.713 | 1.00 | 32.48 | D |
| ATOM | 9112 | CD | LYS D | 724 | 122.379 | −4.944 | 20.947 | 1.00 | 34.54 | D |
| ATOM | 9113 | CE | LYS D | 724 | 122.796 | −4.163 | 19.693 | 1.00 | 36.42 | D |
| ATOM | 9114 | NZ | LYS D | 724 | 123.789 | −4.878 | 18.817 | 1.00 | 36.97 | D |
| ATOM | 9115 | C | LYS D | 724 | 122.373 | −5.535 | 24.346 | 1.00 | 29.03 | D |
| ATOM | 9116 | O | LYS D | 724 | 122.417 | −6.749 | 24.142 | 1.00 | 28.40 | D |
| ATOM | 9117 | N | THR D | 725 | 123.409 | −4.820 | 24.759 | 1.00 | 28.23 | D |
| ATOM | 9118 | CA | THR D | 725 | 124.720 | −5.400 | 24.964 | 1.00 | 27.95 | D |
| ATOM | 9119 | CB | THR D | 725 | 125.784 | −4.578 | 24.178 | 1.00 | 27.43 | D |
| ATOM | 9120 | OG1 | THR D | 725 | 125.351 | −4.417 | 22.821 | 1.00 | 26.72 | D |
| ATOM | 9121 | CG2 | THR D | 725 | 127.134 | −5.273 | 24.189 | 1.00 | 25.82 | D |
| ATOM | 9122 | C | THR D | 725 | 125.106 | −5.460 | 26.443 | 1.00 | 27.90 | D |
| ATOM | 9123 | O | THR D | 725 | 124.891 | −4.505 | 27.199 | 1.00 | 28.70 | D |
| ATOM | 9124 | N | LEU D | 726 | 125.682 | −6.591 | 26.842 | 1.00 | 26.83 | D |
| ATOM | 9125 | CA | LEU D | 726 | 126.119 | −6.806 | 28.213 | 1.00 | 24.94 | D |
| ATOM | 9126 | CB | LEU D | 726 | 125.126 | −7.703 | 28.937 | 1.00 | 27.71 | D |
| ATOM | 9127 | CG | LEU D | 726 | 125.632 | −8.393 | 30.211 | 1.00 | 29.98 | D |
| ATOM | 9128 | CD1 | LEU D | 726 | 126.114 | −7.390 | 31.271 | 1.00 | 30.93 | D |
| ATOM | 9129 | CD2 | LEU D | 726 | 124.478 | −9.202 | 30.758 | 1.00 | 31.05 | D |
| ATOM | 9130 | C | LEU D | 726 | 127.485 | −7.441 | 28.245 | 1.00 | 22.94 | D |
| ATOM | 9131 | O | LEU D | 726 | 127.673 | −8.537 | 27.741 | 1.00 | 24.07 | D |
| ATOM | 9132 | N | VAL D | 727 | 128.436 | −6.753 | 28.856 | 1.00 | 23.62 | D |
| ATOM | 9133 | CA | VAL D | 727 | 129.798 | −7.256 | 28.940 | 1.00 | 23.62 | D |
| ATOM | 9134 | CB | VAL D | 727 | 130.766 | −6.218 | 28.377 | 1.00 | 24.57 | D |
| ATOM | 9135 | CG1 | VAL D | 727 | 132.189 | −6.737 | 28.490 | 1.00 | 28.26 | D |
| ATOM | 9136 | CG2 | VAL D | 727 | 130.414 | −5.939 | 26.896 | 1.00 | 22.22 | D |
| ATOM | 9137 | C | VAL D | 727 | 130.214 | −7.625 | 30.360 | 1.00 | 21.59 | D |
| ATOM | 9138 | O | VAL D | 727 | 129.738 | −7.034 | 31.333 | 1.00 | 20.77 | D |
| ATOM | 9139 | N | ALA D | 728 | 131.106 | −8.608 | 30.461 | 1.00 | 21.47 | D |
| ATOM | 9140 | CA | ALA D | 728 | 131.597 | −9.084 | 31.751 | 1.00 | 21.74 | D |
| ATOM | 9141 | CB | ALA D | 728 | 130.593 | −10.057 | 32.371 | 1.00 | 23.07 | D |
| ATOM | 9142 | C | ALA D | 728 | 132.952 | −9.749 | 31.692 | 1.00 | 21.99 | D |
| ATOM | 9143 | O | ALA D | 728 | 133.199 | −10.624 | 30.846 | 1.00 | 17.98 | D |
| ATOM | 9144 | N | ALA D | 729 | 133.823 | −9.315 | 32.605 | 1.00 | 24.10 | D |
| ATOM | 9145 | CA | ALA D | 729 | 135.175 | −9.868 | 32.740 | 1.00 | 25.79 | D |
| ATOM | 9146 | CB | ALA D | 729 | 136.139 | −8.792 | 33.225 | 1.00 | 26.49 | D |
| ATOM | 9147 | C | ALA D | 729 | 135.036 | −10.980 | 33.770 | 1.00 | 26.20 | D |
| ATOM | 9148 | O | ALA D | 729 | 134.619 | −10.736 | 34.900 | 1.00 | 24.79 | D |
| ATOM | 9149 | N | VAL D | 730 | 135.363 | −12.199 | 33.351 | 1.00 | 27.91 | D |
| ATOM | 9150 | CA | VAL D | 730 | 135.224 | −13.396 | 34.182 | 1.00 | 28.52 | D |
| ATOM | 9151 | CB | VAL D | 730 | 134.162 | −14.349 | 33.578 | 1.00 | 25.65 | D |
| ATOM | 9152 | CD1 | VAL D | 730 | 132.846 | −13.619 | 33.401 | 1.00 | 21.69 | D |
| ATOM | 9153 | CD2 | VAL D | 730 | 134.667 | −14.910 | 32.251 | 1.00 | 23.86 | D |
| ATOM | 9154 | C | VAL D | 730 | 136.496 | −14.215 | 34.373 | 1.00 | 30.60 | D |
| ATOM | 9155 | O | VAL D | 730 | 137.544 | −13.945 | 33.773 | 1.00 | 29.25 | D |
| ATOM | 9156 | N | GLU D | 731 | 136.373 | −15.227 | 35.224 | 1.00 | 34.05 | D |
| ATOM | 9157 | CA | GLU D | 731 | 137.468 | −16.136 | 35.507 | 1.00 | 38.43 | D |
| ATOM | 9158 | CB | GLU D | 731 | 138.069 | −15.875 | 36.877 | 1.00 | 39.08 | D |
| ATOM | 9159 | CG | GLU D | 731 | 139.163 | −16.851 | 37.195 | 1.00 | 41.06 | D |
| ATOM | 9160 | CD | GLU D | 731 | 139.469 | −16.908 | 38.664 | 1.00 | 44.19 | D |
| ATOM | 9161 | OE1 | GLU D | 731 | 139.986 | −15.898 | 39.204 | 1.00 | 43.94 | D |
| ATOM | 9162 | OE2 | GLU D | 731 | 139.184 | −17.969 | 39.275 | 1.00 | 45.15 | D |
| ATOM | 9163 | C | GLU D | 731 | 136.915 | −17.541 | 35.481 | 1.00 | 39.52 | D |
| ATOM | 9164 | O | GLU D | 731 | 136.003 | −17.862 | 36.239 | 1.00 | 38.90 | D |
| ATOM | 9165 | N | LYS D | 732 | 137.464 | −18.375 | 34.606 | 1.00 | 42.02 | D |
| ATOM | 9166 | CA | LYS D | 732 | 137.000 | −19.744 | 34.487 | 1.00 | 44.14 | D |
| ATOM | 9167 | CB | LYS D | 732 | 136.079 | −19.864 | 33.279 | 1.00 | 45.79 | D |
| ATOM | 9168 | CG | LYS D | 732 | 135.263 | −21.130 | 33.269 | 1.00 | 48.52 | D |
| ATOM | 9169 | CD | LYS D | 732 | 134.329 | −21.169 | 32.074 | 1.00 | 48.95 | D |
| ATOM | 9170 | CE | LYS D | 732 | 133.753 | −22.555 | 31.897 | 1.00 | 50.73 | D |
| ATOM | 9171 | NZ | LYS D | 732 | 132.777 | −22.576 | 30.795 | 1.00 | 53.14 | D |
| ATOM | 9172 | C | LYS D | 732 | 138.155 | −20.724 | 34.366 | 1.00 | 45.13 | D |
| ATOM | 9173 | O | LYS D | 732 | 139.012 | −20.593 | 33.491 | 1.00 | 46.00 | D |
| ATOM | 9174 | N | ASP D | 733 | 138.179 | −21.702 | 35.263 | 1.00 | 45.68 | D |
| ATOM | 9175 | CA | ASP D | 733 | 139.227 | −22.710 | 35.263 | 1.00 | 45.99 | D |
| ATOM | 9176 | CE | ASP D | 733 | 139.114 | −23.590 | 34.013 | 1.00 | 47.96 | D |
| ATOM | 9177 | CG | ASP D | 733 | 137.759 | −24.322 | 33.917 | 1.00 | 51.97 | D |
| ATOM | 9178 | OD1 | ASP D | 733 | 137.355 | −24.977 | 34.905 | 1.00 | 55.70 | D |
| ATOM | 9179 | OD2 | ASP D | 733 | 137.095 | −24.256 | 32.855 | 1.00 | 50.55 | D |
| ATOM | 9180 | C | ASP D | 733 | 140.603 | −22.066 | 35.311 | 1.00 | 45.97 | D |
| ATOM | 9181 | O | ASP D | 733 | 141.493 | −22.456 | 34.565 | 1.00 | 46.45 | D |
| ATOM | 9182 | N | GLY D | 734 | 140.778 | −21.075 | 36.182 | 1.00 | 45.60 | D |
| ATOM | 9183 | CA | GLY D | 734 | 142.072 | −20.420 | 36.295 | 1.00 | 44.79 | D |
| ATOM | 9184 | C | GLY D | 734 | 142.438 | −19.447 | 35.182 | 1.00 | 44.98 | D |
| ATOM | 9185 | O | GLY D | 734 | 143.607 | −19.089 | 35.012 | 1.00 | 44.45 | D |
| ATOM | 9186 | N | GLN D | 735 | 141.453 | −19.010 | 34.411 | 1.00 | 43.41 | D |
| ATOM | 9187 | CA | GLN D | 735 | 141.747 | −18.067 | 33.349 | 1.00 | 42.41 | D |
| ATOM | 9188 | CB | GLN D | 735 | 141.935 | −18.820 | 32.034 | 1.00 | 44.33 | D |
| ATOM | 9189 | CG | GLN D | 735 | 143.185 | −19.679 | 32.036 | 1.00 | 46.62 | D |
| ATOM | 9190 | CD | GLN D | 735 | 143.303 | −20.555 | 30.810 | 1.00 | 48.33 | D |
| ATOM | 9191 | OE1 | GLN D | 735 | 143.228 | −20.076 | 29.670 | 1.00 | 49.33 | D |
| ATOM | 9192 | NE2 | GLN D | 735 | 143.495 | −21.853 | 31.035 | 1.00 | 48.62 | D |
| ATOM | 9193 | C | GLN D | 735 | 140.647 | −17.025 | 33.243 | 1.00 | 40.32 | D |
| ATOM | 9194 | O | GLN D | 735 | 139.504 | −17.282 | 33.609 | 1.00 | 40.35 | D |
| ATOM | 9195 | N | SER D | 736 | 141.001 | −15.841 | 32.765 | 1.00 | 37.70 | D |
| ATOM | 9196 | CA | SER D | 736 | 140.033 | −14.770 | 32.630 | 1.00 | 35.67 | D |
| ATOM | 9197 | CB | SER D | 736 | 140.664 | −13.454 | 33.051 | 1.00 | 35.55 | D |
| ATOM | 9198 | OG | SER D | 736 | 141.815 | −13.209 | 32.262 | 1.00 | 33.28 | D |
| ATOM | 9199 | C | SER D | 736 | 139.580 | −14.656 | 31.191 | 1.00 | 35.08 | D |
| ATOM | 9200 | O | SER D | 736 | 140.375 | −14.823 | 30.273 | 1.00 | 35.01 | D |
| ATOM | 9201 | N | PHE D | 737 | 138.299 | −14.370 | 30.998 | 1.00 | 35.00 | D |
| ATOM | 9202 | CA | PHE D | 737 | 137.749 | −14.215 | 29.662 | 1.00 | 34.26 | D |
| ATOM | 9203 | CB | PHE D | 737 | 136.928 | −15.436 | 29.259 | 1.00 | 34.41 | D |
| ATOM | 9204 | CG | PHE D | 737 | 137.736 | −16.689 | 29.121 | 1.00 | 34.85 | D |
| ATOM | 9205 | CD1 | PHE D | 737 | 138.122 | −17.416 | 30.243 | 1.00 | 34.24 | D |
| ATOM | 9206 | CD2 | PHE D | 737 | 138.120 | −17.140 | 27.864 | 1.00 | 34.84 | D |
| ATOM | 9207 | CE1 | PHE D | 737 | 138.871 | −18.574 | 30.108 | 1.00 | 34.16 | D |
| ATOM | 9208 | CE2 | PHE D | 737 | 138.868 | −18.293 | 27.724 | 1.00 | 33.97 | D |
| ATOM | 9209 | CZ | PHE D | 737 | 139.246 | −19.012 | 28.845 | 1.00 | 34.14 | D |
| ATOM | 9210 | C | PHE D | 737 | 136.869 | −12.992 | 29.611 | 1.00 | 33.97 | D |
| ATOM | 9211 | O | PHE D | 737 | 136.627 | −12.359 | 30.639 | 1.00 | 34.16 | D |
| ATOM | 9212 | N | LEU D | 738 | 136.387 | −12.673 | 28.411 | 1.00 | 32.98 | D |
| ATOM | 9213 | CA | LEU D | 738 | 135.526 | −11.514 | 28.199 | 1.00 | 33.21 | D |
| ATOM | 9214 | CB | LEU D | 738 | 136.222 | −10.527 | 27.255 | 1.00 | 32.13 | D |
| ATOM | 9215 | CG | LEU D | 738 | 135.744 | −9.075 | 27.237 | 1.00 | 32.97 | D |
| ATOM | 9216 | CD1 | LEU D | 738 | 134.465 | −8.962 | 26.461 | 1.00 | 33.24 | D |
| ATOM | 9217 | CD2 | LEU D | 738 | 135.574 | −8.569 | 28.668 | 1.00 | 33.16 | D |
| ATOM | 9218 | C | LEU D | 738 | 134.180 | −11.955 | 27.620 | 1.00 | 33.76 | D |
| ATOM | 9219 | O | LEU D | 738 | 134.110 | −12.360 | 26.473 | 1.00 | 36.66 | D |
| ATOM | 9220 | N | GLU D | 739 | 133.114 | −11.870 | 28.410 | 1.00 | 33.64 | D |
| ATOM | 9221 | CA | GLU D | 739 | 131.793 | −12.292 | 27.959 | 1.00 | 32.51 | D |
| ATOM | 9222 | CB | GLU D | 739 | 131.000 | −12.884 | 29.121 | 1.00 | 33.25 | D |
| ATOM | 9223 | CG | GLU D | 739 | 131.529 | −14.166 | 29.668 | 1.00 | 34.95 | D |
| ATOM | 9224 | CD | GLU D | 739 | 131.644 | −15.235 | 28.602 | 1.00 | 38.68 | D |
| ATOM | 9225 | OE1 | GLU D | 739 | 130.731 | −15.323 | 27.733 | 1.00 | 38.32 | D |
| ATOM | 9226 | OE2 | GLU D | 739 | 132.651 | −15.991 | 28.646 | 1.00 | 39.63 | D |
| ATOM | 9227 | C | GLU D | 739 | 130.961 | −11.163 | 27.377 | 1.00 | 32.88 | D |
| ATOM | 9228 | O | GLU D | 739 | 130.735 | −10.144 | 28.044 | 1.00 | 33.94 | D |
| ATOM | 9229 | N | ILE D | 740 | 130.487 | −11.329 | 26.147 | 1.00 | 31.70 | D |
| ATOM | 9230 | CA | ILE D | 740 | 129.641 | −10.301 | 25.568 | 1.00 | 29.64 | D |
| ATOM | 9231 | CB | ILE D | 740 | 130.288 | −9.670 | 24.299 | 1.00 | 28.27 | D |
| ATOM | 9232 | CG2 | ILE D | 740 | 129.461 | −8.484 | 23.831 | 1.00 | 27.08 | D |
| ATOM | 9233 | CG1 | ILE D | 740 | 131.663 | −9.093 | 24.620 | 1.00 | 24.97 | D |
| ATOM | 9234 | CD1 | ILE D | 740 | 132.282 | −8.385 | 23.424 | 1.00 | 24.47 | D |
| ATOM | 9235 | C | ILE D | 740 | 128.275 | −10.906 | 25.252 | 1.00 | 29.65 | D |
| ATOM | 9236 | O | ILE D | 740 | 128.174 | −11.845 | 24.470 | 1.00 | 30.71 | D |
| ATOM | 9237 | N | LEU D | 741 | 127.226 | −10.388 | 25.885 | 1.00 | 30.03 | D |
| ATOM | 9238 | CA | LEU D | 741 | 125.872 | −10.903 | 25.659 | 1.00 | 30.25 | D |
| ATOM | 9239 | CB | LEU D | 741 | 125.209 | −11.213 | 26.994 | 1.00 | 30.19 | D |
| ATOM | 9240 | CG | LEU D | 741 | 126.016 | −12.176 | 27.872 | 1.00 | 30.17 | D |
| ATOM | 9241 | CD1 | LEU D | 741 | 125.458 | −12.120 | 29.284 | 1.00 | 31.69 | D |
| ATOM | 9242 | CD2 | LEU D | 741 | 125.969 | −13.591 | 27.323 | 1.00 | 26.90 | D |
| ATOM | 9243 | C | LEU D | 741 | 125.012 | −9.935 | 24.863 | 1.00 | 29.80 | D |
| ATOM | 9244 | O | LEU D | 741 | 124.939 | −8.750 | 25.176 | 1.00 | 28.77 | D |
| ATOM | 9245 | N | ASP D | 742 | 124.354 | −10.462 | 23.837 | 1.00 | 31.56 | D |
| ATOM | 9246 | CA | ASP D | 742 | 123.513 | −9.664 | 22.948 | 1.00 | 32.74 | D |
| ATOM | 9247 | CB | ASP D | 742 | 123.917 | −9.906 | 21.492 | 1.00 | 33.83 | D |
| ATOM | 9248 | CG | ASP D | 742 | 123.601 | −8.730 | 20.592 | 1.00 | 34.30 | D |
| ATOM | 9249 | OD1 | ASP D | 742 | 122.422 | −8.315 | 20.526 | 1.00 | 33.76 | D |
| ATOM | 9250 | OD2 | ASP D | 742 | 124.550 | −8.223 | 19.952 | 1.00 | 34.48 | D |
| ATOM | 9251 | C | ASP D | 742 | 122.046 | −10.021 | 23.110 | 1.00 | 33.39 | D |
| ATOM | 9252 | O | ASP D | 742 | 121.617 | −11.102 | 22.713 | 1.00 | 33.45 | D |
| ATOM | 9253 | N | PHE D | 743 | 121.283 | −9.097 | 23.685 | 1.00 | 34.50 | D |
| ATOM | 9254 | CA | PHE D | 743 | 119.858 | −9.296 | 23.905 | 1.00 | 34.56 | D |
| ATOM | 9255 | CB | PHE D | 743 | 119.422 | −8.544 | 25.161 | 1.00 | 32.05 | D |
| ATOM | 9256 | CG | PHE D | 743 | 119.627 | −9.324 | 26.400 | 1.00 | 29.59 | D |
| ATOM | 9257 | CD1 | PHE D | 743 | 120.893 | −9.462 | 26.941 | 1.00 | 27.43 | D |
| ATOM | 9258 | CD2 | PHE D | 743 | 118.579 | −10.058 | 26.935 | 1.00 | 28.98 | D |
| ATOM | 9259 | CE1 | PHE D | 743 | 121.116 | −10.332 | 27.987 | 1.00 | 26.97 | D |
| ATOM | 9260 | CE2 | PHE D | 743 | 118.787 | −10.933 | 27.980 | 1.00 | 27.93 | D |
| ATOM | 9261 | CZ | PHE D | 743 | 120.059 | −11.077 | 28.507 | 1.00 | 26.94 | D |
| ATOM | 9262 | C | PHE D | 743 | 118.973 | −8.895 | 22.734 | 1.00 | 35.12 | D |
| ATOM | 9263 | O | PHE D | 743 | 117.746 | −8.889 | 22.849 | 1.00 | 35.80 | D |
| ATOM | 9264 | N | SER D | 744 | 119.594 | −8.592 | 21.600 | 1.00 | 35.35 | D |
| ATOM | 9265 | CA | SER D | 744 | 118.854 | −8.176 | 20.416 | 1.00 | 35.76 | D |
| ATOM | 9266 | CB | SER D | 744 | 119.823 | −7.612 | 19.381 | 1.00 | 36.40 | D |
| ATOM | 9267 | OG | SER D | 744 | 119.126 | −6.857 | 18.417 | 1.00 | 40.54 | D |
| ATOM | 9268 | C | SER D | 744 | 118.029 | −9.301 | 19.793 | 1.00 | 35.75 | D |
| ATOM | 9269 | O | SER D | 744 | 118.433 | −10.488 | 19.873 | 1.00 | 36.28 | D |
| ATOM | 9270 | OXT | SER D | 744 | 116.984 | −8.972 | 19.209 | 1.00 | 34.82 | D |
| ATOM | 9271 | CB | LEU E | 2 | 57.203 | −3.013 | 76.696 | 1.00 | 93.40 | E |
| ATOM | 9272 | CG | LEU E | 2 | 56.802 | −2.909 | 78.154 | 1.00 | 92.64 | E |
| ATOM | 9273 | CD1 | LEU E | 2 | 56.089 | −4.209 | 78.494 | 1.00 | 92.62 | E |
| ATOM | 9274 | CD2 | LEU E | 2 | 58.002 | −2.716 | 79.063 | 1.00 | 91.70 | E |
| ATOM | 9275 | C | LEU E | 2 | 58.910 | −2.837 | 74.950 | 1.00 | 94.06 | E |
| ATOM | 9276 | O | LEU E | 2 | 59.052 | −4.056 | 75.064 | 1.00 | 94.80 | E |
| ATOM | 9277 | N | LEU E | 2 | 57.498 | −0.813 | 75.549 | 1.00 | 93.79 | E |
| ATOM | 9278 | CA | LEU E | 2 | 58.180 | −2.054 | 76.029 | 1.00 | 93.74 | E |
| ATOM | 9279 | N | LEU E | 3 | 59.349 | −2.162 | 73.890 | 1.00 | 93.32 | E |
| ATOM | 9280 | CA | LEU E | 3 | 60.093 | −2.853 | 72.840 | 1.00 | 91.84 | E |
| ATOM | 9281 | CS | LEU E | 3 | 60.099 | −2.030 | 71.539 | 1.00 | 90.21 | E |
| ATOM | 9282 | CG | LEU E | 3 | 58.738 | −2.011 | 70.818 | 1.00 | 88.51 | E |
| ATOM | 9283 | CD1 | LEU E | 3 | 58.863 | −1.419 | 69.431 | 1.00 | 86.61 | E |
| ATOM | 9284 | CD2 | LEU E | 3 | 58.194 | −3.426 | 70.729 | 1.00 | 87.78 | E |
| ATOM | 9285 | C | LEU E | 3 | 61.504 | −3.132 | 73.355 | 1.00 | 91.93 | E |
| ATOM | 9286 | O | LEU E | 3 | 62.459 | −2.428 | 73.011 | 1.00 | 92.22 | E |
| ATOM | 9287 | N | TPO E | 4 | 61.634 | −4.171 | 74.204 | 1.00 | 91.56 | E |
| ATOM | 9288 | CA | TPO E | 4 | 62.536 | −4.975 | 75.032 | 1.00 | 90.52 | E |
| ATOM | 9289 | CE | TPO E | 4 | 62.853 | −4.058 | 76.249 | 1.00 | 89.37 | E |
| ATOM | 9290 | CG2 | TPO E | 4 | 63.717 | −4.740 | 77.314 | 1.00 | 89.39 | E |
| ATOM | 9291 | OG1 | TPO E | 4 | 63.546 | −2.864 | 75.833 | 1.00 | 86.87 | E |
| ATOM | 9292 | P | TPO E | 4 | 65.016 | −2.884 | 75.092 | 1.00 | 83.35 | E |
| ATOM | 9293 | O1P | TPO E | 4 | 64.849 | −3.624 | 73.731 | 1.00 | 83.36 | E |
| ATOM | 9294 | O2P | TPO E | 4 | 66.088 | −3.641 | 76.006 | 1.00 | 83.80 | E |
| ATOM | 9295 | O3P | TPO E | 4 | 65.359 | −1.340 | 74.831 | 1.00 | 84.80 | E |
| ATOM | 9296 | C | TPO E | 4 | 61.804 | −6.259 | 75.507 | 1.00 | 91.34 | E |
| ATOM | 9297 | O | TPO E | 4 | 60.467 | −6.316 | 75.619 | 1.00 | 91.46 | E |
| ATOM | 9298 | N | PRO E | 5 | 63.256 | −5.997 | 71.948 | 1.00 | 83.51 | E |
| ATOM | 9299 | CD | PRO E | 5 | 63.441 | −4.606 | 71.529 | 1.00 | 83.14 | E |
| ATOM | 9300 | CA | PRO E | 5 | 63.931 | −6.885 | 71.003 | 1.00 | 84.53 | E |
| ATOM | 9301 | CE | PRO E | 5 | 65.159 | −6.089 | 70.575 | 1.00 | 83.61 | E |
| ATOM | 9302 | OG | PRO E | 5 | 64.831 | −4.637 | 70.919 | 1.00 | 83.06 | E |
| ATOM | 9303 | C | PRO E | 5 | 64.316 | −8.236 | 71.564 | 1.00 | 85.50 | E |
| ATOM | 9304 | O | PRO E | 5 | 64.916 | −8.330 | 72.628 | 1.00 | 85.67 | E |
| ATOM | 9305 | N | PRO E | 6 | 63.969 | −9.312 | 70.847 | 1.00 | 86.93 | E |
| ATOM | 9306 | CD | PRO E | 6 | 63.085 | −9.303 | 69.669 | 1.00 | 87.22 | E |
| ATOM | 9307 | CA | PRO E | 6 | 64.267 | −10.690 | 71.255 | 1.00 | 88.33 | E |
| ATOM | 9308 | CE | PRO E | 6 | 63.629 | −11.522 | 70.149 | 1.00 | 87.80 | E |
| ATOM | 9309 | CG | PRO E | 6 | 62.478 | −10.688 | 69.722 | 1.00 | 87.62 | E |
| ATOM | 9310 | C | PRO E | 6 | 65.765 | −10.988 | 71.384 | 1.00 | 89.85 | E |
| ATOM | 9311 | O | PRO E | 6 | 66.575 | −10.544 | 70.557 | 1.00 | 90.61 | E |
| ATOM | 9312 | N | GLN E | 7 | 66.126 | −11.745 | 72.421 | 1.00 | 90.83 | E |
| ATOM | 9313 | CA | GLN E | 7 | 67.515 | −12.125 | 72.626 | 1.00 | 91.45 | E |
| ATOM | 9314 | CE | GLN E | 7 | 68.006 | −11.682 | 73.992 | 1.00 | 91.16 | E |
| ATOM | 9315 | CG | GLN E | 7 | 68.555 | −10.282 | 73.957 | 1.00 | 91.12 | E |
| ATOM | 9316 | CD | GLN E | 7 | 69.842 | −10.165 | 74.739 | 1.00 | 91.14 | E |
| ATOM | 9317 | OE1 | GLN E | 7 | 69.851 | −10.287 | 75.975 | 1.00 | 92.25 | E |
| ATOM | 9318 | NB2 | GLN E | 7 | 70.950 | −9.958 | 74.025 | 1.00 | 89.30 | E |
| ATOM | 9319 | C | GLN E | 7 | 67.666 | −13.631 | 72.485 | 1.00 | 91.90 | E |
| ATOM | 9320 | 0 | GLN E | 7 | 67.429 | −14.383 | 73.431 | 1.00 | 92.11 | E |
| ATOM | 9321 | N | SER E | 8 | 68.051 | −14.055 | 71.282 | 1.00 | 92.25 | E |
| ATOM | 9322 | CA | SER E | 8 | 68.235 | −15.467 | 70.933 | 1.00 | 92.14 | E |
| ATOM | 9323 | CE | SER E | 8 | 67.113 | −15.896 | 69.981 | 1.00 | 92.25 | E |
| ATOM | 9324 | OG | SER E | 8 | 66.978 | −17.305 | 69.938 | 1.00 | 92.67 | E |
| ATOM | 9325 | C | SER E | 8 | 69.603 | −15.640 | 70.258 | 1.00 | 92.02 | E |
| ATOM | 9326 | O | SER E | 8 | 70.638 | −15.398 | 70.879 | 1.00 | 91.94 | E |
| ATOM | 9327 | N | GLY E | 9 | 69.602 | −16.057 | 68.994 | 1.00 | 91.78 | E |
| ATOM | 9328 | CA | GLY E | 9 | 70.854 | −16.235 | 68.277 | 1.00 | 91.39 | E |
| ATOM | 9329 | C | GLY E | 9 | 70.848 | −15.537 | 66.928 | 1.00 | 91.23 | E |
| ATOM | 9330 | O | GLY E | 9 | 69.743 | −15.212 | 66.445 | 1.00 | 90.99 | E |
| ATOM | 9331 | OXT | GLY E | 9 | 71.939 | −15.322 | 66.346 | 1.00 | 90.21 | E |
| ATOM | 9332 | CE | LEU F | 2 | 137.489 | −5.702 | 48.818 | 1.00 | 79.71 | F |
| ATOM | 9333 | CG | LEU F | 2 | 138.842 | −5.743 | 49.546 | 1.00 | 80.18 | F |
| ATOM | 9334 | CD1 | LEU F | 2 | 139.667 | −4.549 | 49.099 | 1.00 | 79.66 | F |
| ATOM | 9335 | CD2 | LEU F | 2 | 138.661 | −5.730 | 51.075 | 1.00 | 78.72 | F |
| ATOM | 9336 | C | LEU F | 2 | 138.487 | −6.493 | 46.630 | 1.00 | 78.95 | F |
| ATOM | 9337 | O | LEU F | 2 | 138.336 | −7.720 | 46.632 | 1.00 | 77.64 | F |
| ATOM | 9338 | N | LEU F | 2 | 136.056 | −5.715 | 46.790 | 1.00 | 79.28 | F |
| ATOM | 9339 | CA | LEU F | 2 | 137.454 | −5.545 | 47.286 | 1.00 | 79.36 | F |
| ATOM | 9340 | N | LEU F | 3 | 139.520 | −5.871 | 46.060 | 1.00 | 78.25 | F |
| ATOM | 9341 | CA | LEU F | 3 | 140.670 | −6.502 | 45.403 | 1.00 | 76.54 | F |
| ATOM | 9342 | CB | LEU F | 3 | 140.433 | −6.740 | 43.915 | 1.00 | 77.32 | F |
| ATOM | 9343 | CG | LEU F | 3 | 139.459 | −7.802 | 43.429 | 1.00 | 78.88 | F |
| ATOM | 9344 | CD1 | LEU F | 3 | 138.100 | −7.190 | 43.081 | 1.00 | 80.14 | F |
| ATOM | 9345 | CD2 | LEU F | 3 | 140.065 | −8.449 | 42.204 | 1.00 | 78.71 | F |
| ATOM | 9346 | C | LEU F | 3 | 141.723 | −5.415 | 45.530 | 1.00 | 75.20 | F |
| ATOM | 9347 | O | LEU F | 3 | 141.444 | −4.264 | 45.195 | 1.00 | 75.82 | F |
| ATOM | 9348 | N | TPO F | 4 | 142.936 | −5.746 | 46.012 | 1.00 | 72.87 | F |
| ATOM | 9349 | CA | TPO F | 4 | 144.008 | −4.734 | 46.163 | 1.00 | 70.82 | F |
| ATOM | 9350 | CB | TPO F | 4 | 144.161 | −4.534 | 47.709 | 1.00 | 68.53 | F |
| ATOM | 9351 | CG2 | TPO F | 4 | 145.393 | −3.748 | 48.146 | 1.00 | 70.46 | F |
| ATOM | 9352 | OG1 | TPO F | 4 | 142.965 | −3.903 | 48.203 | 1.00 | 65.51 | F |
| ATOM | 9353 | P | TPO F | 4 | 142.778 | −2.260 | 48.081 | 1.00 | 60.19 | F |
| ATOM | 9354 | O1P | TPO F | 4 | 142.871 | −1.889 | 46.565 | 1.00 | 64.16 | F |
| ATOM | 9355 | O2P | TPO F | 4 | 143.905 | −1.501 | 48.889 | 1.00 | 62.63 | F |
| ATOM | 9356 | O3P | TPO F | 4 | 141.319 | −1.975 | 48.672 | 1.00 | 64.49 | F |
| ATOM | 9357 | C | TPO F | 4 | 145.311 | −5.244 | 45.579 | 1.00 | 70.94 | F |
| ATOM | 9358 | O | TPO F | 4 | 145.629 | −6.541 | 45.562 | 1.00 | 71.31 | F |
| ATOM | 9359 | N | PRO F | 5 | 146.116 | −4.278 | 45.081 | 1.00 | 71.04 | F |
| ATOM | 9360 | CD | PRO F | 5 | 145.651 | −2.886 | 44.895 | 1.00 | 69.22 | F |
| ATOM | 9361 | CA | PRO F | 5 | 147.434 | −4.439 | 44.468 | 1.00 | 71.37 | F |
| ATOM | 9362 | CB | PRO F | 5 | 147.722 | −3.051 | 43.898 | 1.00 | 70.57 | F |
| ATOM | 9363 | CG | PRO F | 5 | 146.379 | −2.473 | 43.673 | 1.00 | 68.58 | F |
| ATOM | 9364 | C | PRO F | 5 | 148.447 | −4.823 | 45.533 | 1.00 | 72.63 | F |
| ATOM | 9365 | O | PRO F | 5 | 148.273 | −4.524 | 46.709 | 1.00 | 72.26 | F |
| ATOM | 9366 | N | PRO F | 6 | 149.531 | −5.484 | 45.133 | 1.00 | 74.58 | F |
| ATOM | 9367 | CD | PRO F | 6 | 149.883 | −5.888 | 43.760 | 1.00 | 74.65 | F |
| ATOM | 9368 | CA | PRO F | 6 | 150.563 | −5.898 | 46.087 | 1.00 | 76.44 | F |
| ATOM | 9369 | CB | PRO F | 6 | 151.636 | −6.499 | 45.180 | 1.00 | 76.17 | F |
| ATOM | 9370 | CG | PRO F | 6 | 150.843 | −7.015 | 44.005 | 1.00 | 75.36 | F |
| ATOM | 9371 | C | PRO F | 6 | 151.108 | −4.721 | 46.907 | 1.00 | 78.12 | F |
| ATOM | 9372 | O | PRO F | 6 | 151.745 | −3.827 | 46.351 | 1.00 | 78.87 | F |
| ATOM | 9373 | N | GLN F | 7 | 150.858 | −4.699 | 48.213 | 1.00 | 79.62 | F |
| ATOM | 9374 | CA | GLN F | 7 | 151.397 | −3.605 | 49.020 | 1.00 | 81.37 | F |
| ATOM | 9375 | CB | GLN F | 7 | 150.607 | −3.435 | 50.333 | 1.00 | 81.68 | F |
| ATOM | 9376 | CG | GLN F | 7 | 151.014 | −2.213 | 51.171 | 1.00 | 82.68 | F |
| ATOM | 9377 | CD | GLN F | 7 | 150.936 | −0.896 | 50.397 | 1.00 | 83.36 | F |
| ATOM | 9378 | OE1 | GLN F | 7 | 151.562 | −0.740 | 49.343 | 1.00 | 84.22 | F |
| ATOM | 9379 | NE2 | GLN F | 7 | 150.175 | 0.061 | 50.927 | 1.00 | 82.82 | F |
| ATOM | 9380 | C | GLN F | 7 | 152.866 | −3.939 | 49.299 | 1.00 | 82.45 | F |
| ATOM | 9381 | O | GLN F | 7 | 153.186 | −4.607 | 50.282 | 1.00 | 82.78 | F |
| ATOM | 9382 | N | SER F | 8 | 153.745 | −3.472 | 48.411 | 1.00 | 83.82 | F |
| ATOM | 9383 | CA | SER F | 8 | 155.196 | −3.704 | 48.479 | 1.00 | 85.47 | F |
| ATOM | 9384 | CB | SER F | 8 | 155.937 | −2.583 | 47.732 | 1.00 | 84.24 | F |
| ATOM | 9385 | OG | SER F | 8 | 155.277 | −2.236 | 46.527 | 1.00 | 83.28 | F |
| ATOM | 9386 | C | SER F | 8 | 155.813 | −3.857 | 49.887 | 1.00 | 86.86 | F |
| ATOM | 9387 | O | SER F | 8 | 156.285 | −2.881 | 50.482 | 1.00 | 87.36 | F |
| ATOM | 9388 | N | GLY F | 9 | 155.830 | −5.093 | 50.390 | 1.00 | 87.52 | F |
| ATOM | 9389 | CA | GLY F | 9 | 156.387 | −5.374 | 51.708 | 1.00 | 87.82 | F |
| ATOM | 9390 | C | GLY F | 9 | 155.445 | −6.117 | 52.646 | 1.00 | 87.94 | F |
| ATOM | 9391 | O | GLY F | 9 | 154.243 | −6.240 | 52.319 | 1.00 | 88.07 | F |
| ATOM | 9392 | OXT | GLY F | 9 | 155.901 | −6.573 | 53.717 | 1.00 | 87.76 | F |
| END | | | | | | | | | | |

TABLE 7
Oligonucleotides used in Example 2

| | | |
|---|---|---|
| MTO# | Oligo name | Sequence |

| | | |
|---|---|---|
| MTO 1146 | LP6DelA-FWD | gccttcgcgaactgatgtacactttgcagggtcatacagc |
| MTO 1147 | LP6DelA-REV | gccttggccattttctagatcccaaattctaatagtggtatccatac |
| MTO 1254 | CDC4-BNSH-F | gtaggatccatatgggcgccgcaagctttcccttagctgagtttcc |
| MTO 1367 | V384N-C | catatgacgagtaatattacgtgcttgc |
| MTO 1368 | V384N3 | caagcacgtaatattactcgtcatatg |
| MTO 1369 | K402A-C | ggggctgatgacgcaatgatcagag |
| MTO 1370 | K402A-N | ctctgatcattgcgtcatcagcccc |
| MTO 1371 | W426A 5′ | gatggtggggttgcggcgctgaagtatgcccatg |
| MTO 1372 | DN426A | catgggcatacttcagcgccgcaaccccaccatc |
| MTO 1373 | R443D-C | ggttctacagacgacacggtccgagtttggg |
| MTO 1374 | R443D | tatcccaaactcggaccgtgtcgtcgtctgtagaaccg |
| MTO 1375 | Y548F | cagtgtattatcaaagcttccactaacgac |
| MTO 1376 | Y548F-C | gtcgttagtggaagctttgataatacactg |
| MTO 1377 | Y574F-N | gtagattgtcgaaaatattcgatccgtatg |
| MTO 1378 | Y574F-C | catacggatcgaatattttcgacaatctac |
| MTO 1379 | W717N Hpa | ttgcccttaaagttaaccgagttaatctgatcag |
| MTO 1380 | W717N-C | ctgatcagattaactcggttaactttaagggcaa |
| MTO 1381 | 2Flex359 5′ | tcttttctggagcccgggccaattttaaaaaattggtac |
| MTO 1382 | 2Flex359 3′ | gtaccaattttttaaaattggcccgggctccagaaaaga |
| MTO 1383 | H5 Dest 5′ | ggttttaattctctcggcccgggaccctcccaaaaatacccaaaactc |
| MTO 1384 | H5 Dest 3′ | gagttttgggtatttttgggagggtcccgggccgagagaattaaaacc |
| MTO 1385 | H5 del 5′ | gtgagcccaaagggtccaaagctttcacaacaagatcgc |
| MTO 1386 | H5 del 3′ | gcgatcttgttgtgaaagctttggaccctttgggctcac |
| MTO 1387 | H5 + Dest 5′ | gtggaaaaaacttctgatattcattttaaaaaattggtacaatcc |
| MTO 1388 | H5 + Dest 3′ | ggattgtaccaattttttaaaatgaatatcagaagttttttccac |
| MTO 1389 | Ala1-3 | ccaattttttaaaatgaatgcaatattctccagaaaag |
| MTO 1390 | Ala2-3 | ccaattttttaaaatgaatgctgcaatattctccagaaaag |
| MTO 1391 | Ala3-3 | ccaatttttttaaaatgaaagcggctgcaatattctccagaaaag |
| MTO 1392 | Ala4-3 | ccaattttttaaaatgaaggctgcagctgcaatattctccagaaaag |
| MTO 1393 | Ala8 5′ | gccgcgcgcgctaaagctgcagcgttcattttaaaaaattggtacaatcc |
| MTO 1394 | Ala8 3′ | cgctgcagctttagcgcgcgcggctatattctccagaaaagataatc |
| MTO 1395 | Ala12 5′ | gccgcgcgcgctaaagctgcagcgcgcgcgaaagccttcattttaaaaaattggtacaatcc |
| MTO 1396 | Al12 3′ | ggctttcgcgcgcgctgcagctttagcgcgcgcggctatattctccagaaaagataatc |

TABLE 8
Plasmids used in Example 2

| Plasmid | Relevant Characteristic | Source |
|---|---|---|
| pMT 3169 | pProEx Hta-CDC4 LP6DelA GST-Skp1 | This study |
| pMT 3055 | pProEx Hta-CDC4 263-744 GST-Skp1 | Nash et. al. |
| pMT 3000 | pProEx Hta-CDC4 1-744 GST-Skp1 | This study |
| pMT 3217 | pRS 314 CDC4-BNSH/Eco | This study |
| pMT 3001 | pMT 3055 V384N | This study |
| pMT 3002 | pMT 3055 K402A | This study |
| pMT 3385 | pMT 3055 W426A | This study |
| pMT 3058 | pMT 3055 R443A | Nash et. al |
| pMT 3003 | pMT 3055 R443D | This study |
| pMT 3059 | pMT 3055 R467A | Nash et. al |
| pMT 3060 | pMT 3055 R485A | Nash et. al |
| pMT 3061 | pMT 3055 R534A | Nash et. al |
| pMT 3004 | pMT 3055 Y548F | This study |
| pMT 3005 | pMT 3055 Y574F | This study |
| pMT 3006 | pMT 3055 W717N | This study |
| pMT 3007 | pMT 3055 V384N + W717N | This study |
| pMT 3008 | pMT 3055 K402 + R443D | This study |
| pMT 3010 | pMT 3000 R443D | This study |
| pMT 3011 | pMT 3000 R443A | This study |
| pMT 3012 | pMT 3000 W717N | This study |
| pMT 3013 | pMT 3000 K402A + R443D | This study |
| pMT 3014 | pMT 3000 V384N + W717N | This study |
| pMT 3015 | pMT 3217 V384N | This study |
| pMT 3016 | pMT 3217 K402A | This study |
| pMT 3386 | pMT 3217 W426A | This study |
| pMT 3017 | pMT 3217 R443D | This study |
| pMT 3058 | PRS 314-CDC4 R443A | Nash et. al. |
| pMT 3018 | pMT 3217 Y548F | This study |
| pMT 3068 | PRS 314-CDC4 R572A | Nash et. al |
| pMT 3019 | pMT 3217 Y574F | This study |
| pMT 3020 | pMT 3217 W717N | This study |
| pMT 3021 | pMT 3217 V384N + W717N | This study |
| pMT 3022 | pMT 3217 K402A + R443D | This study |
| pMT 3024 | pMT 3000 Helix 5 Δ 330-443 | This study |
| pMT 3025 | pMT 3000 Helix 5 breaker | This study |
| pMT 3026 | pMT 3000 Helix 5 Δ 321-360 | This study |
| pMT 3027 | pMT 3000 Helix 6 breaker | This study |
| pMT 3028 | pMT 3000 Ala1 insert | This study |
| pMT 3029 | pMT 3000 Ala2 insert | This study |
| pMT 3030 | pMT 3000 Ala3 insert | This study |
| pMT 3031 | pMT 3000 Ala4 insert | This study |
| pMT 3032 | pMT 3000 Ala8 insert | This study |
| pMT 3033 | pMT 3000 Ala12 insert | This study |
| pMT 3034 | pMT 3217 Helix 5 Δ 330-443 | This study |
| pMT 3035 | pMT 3217 Helix 5 breaker | This study |
| pMT 3036 | pMT 3217 Helix 5 Δ 321-360 | This study |
| pMT 3037 | pMT 3217 Helix 6 breaker | This study |
| pMT 3038 | pMT 3217 Ala1 insert | This study |
| pMT 3039 | pMT 3217 Ala2 insert | This study |
| pMT 3040 | pMT 3217 Ala3 insert | This study |
| pMT 3041 | pMT 3217 Ala4 insert | This study |
| pMT 3042 | pMT 3217 Ala8 insert | This study |
| pMT 3043 | pMT 3217 Ala12 insert | This study |
| pMT 1571 | Pet16b-Sic1 | Previous study |
| MDM 152 | pGALl1 SIC1 LEU2 CEN ARS | M. Mendenhall |
| MDM202 | pGAL1 SIC1^{T33V} LEU2 CEN ARS | M. Mendenhall |

### LIST OF REFERENCES

1. Amati, B., and Vlach, J. (1999). Kip1 meets SKP2: new links in cell-cycle control [news], Nat Cell Biol 1, E91-3.

2. Bai, C., Sen, P., Hofmann, K., Ma, L., Goebl, M., Harper, J. W., and Elledge, S. J. (1996). SKP1 connects cell cycle regulators to the ubiquitin proteolysis machinery through a novel motif, the F-box, Cell 86, 263-74.

3. Brunger, A. T., Adams, P. D., Clore, G. M., Gros, P., Grosse-Kuntsleve, Jiang, R. W., Kuszewski, J., Nilges, M., Pannu, N. S., Read, R. J., and al., e. (1998). Crystallography and NMR system: a new software suite for macromolecular structure determination. Acta Cryst. D54, 905-921.

4. Carson, M. (1991). Ribbons 2.0. J. Appl. Crystallgr. 24, 958-961.

5. Chi, Y., Huddleston, M. J., Zhang, X., Young, R. A., Annan, R. S., Carr, S. A., and Deshaies, R. J. (2001). Negative regulation of Gcn4 and Msn2 transcription factors by Srb10 cyclin-dependent kinase. Genes. Dev. 15, 1078-1092.

6. Cohen, P. (2000). The regulation of protein function by multisite phosphorylation: a 25 year update. Trends Biochem. Sci. 25, 596-601.

7. de La Fortelle, E., and Bricogne, G. (1997). Maximum-likelihood heavy-atom parameter refinement for multiple isomorphous replacement and multiwavelength anomalous diffraction methods. Methods Enzymol. 276, 472-494.

8. Deshaies, R. J. (1999). SCF and Cullin/Ring H2-based ubiquitin ligases, Annu Rev Cell Dev Biol 15, 435-67.

9. Deshaies, R. J., and Ferrell, J. E., Jr. (2001). Multisite phosphorylation and the countdown to S phase, Cell 107, 819-22.

10. Drury, L. S., Perkins, G., and Diffley, J. F. (1997). The Cdc4/34/53 pathway targets Cdc6p for proteolysis in budding yeast, Embo J 16, 5966-76.

11. Eck, M. J., Pluskey, S., Trub, T., Harrison, S. C., and Shoelson, S. E. (1996). Spatial constraints on the recognition of phosphoproteins by the tandem SH2 domains of the phosphatase SH-PTP2, Nature 379, 277-80.

12. Endicott, J. A., Noble, M. E., and Tucker, J. A. (1999). Cyclin-dependent kinases: inhibition and substrate recognition. Curr. Opin. Struct. Biol. 9, 738-744.

13. Feldman, R. M., Correll, C. C., Kaplan, K. B., and Deshaies, R. J. (1997). A complex of Cdc4p, Skp1p, and Cdc53p/cullin catalyzes ubiquitination of the phosphorylated CDK inhibitor Sic1p, Cell 91, 221-30.

14. Ferrell, J. E. (1996). Tripping the switch fantastic: how a protein kinase cascade can convert graded inputs into switch-like outputs, Trends Biochem Sci 21, 460-466.

15. Fulop, V., and Jones, D. T. (1999). Beta propellers: structural rigidity and functional diversity, Curr Opin Struct Biol 9, 715-721.

16. Gstaiger, M., Jordan, R., Lim, M., Catzavelos, C., Mestan, J., Slingerland, J., and Krek, W. (2001). Skp2 is oncogenic and overexpressed in human cancers, Proc Natl Acad Sci USA 98, 5043-8.

17. Gupta-Rossi, N., Le Bail, O., Gonen, H., Brou, C., Logeat, F., Six, E., Ciechanover, A., and Israel, A. (2002). Functional interaction between SEL-10, an F-box protein, and the nuclear form of activated Notch1 receptor, J Biol Chem 276, 34371-34378.

18. Hamilton, K. S., Ellison, M. J., Barber, K. R., Williams, R. S., Huzil, J. T., McKenna, S., Ptak, C., Glover, M., and Shaw, G. S. (2001). Structure of a conjugating enzyme-ubiquitin thiolester intermediate reveals a novel role for the ubiquitin tail. Structure 9, 897-904.

19. Harper, J. W. (2001). Protein destruction: adapting roles for Cks proteins. Curr Biol 11, R431-435.

20. Harper, J. W. (2002). A phosphorylation-driven ubiquitination switch for cell-cycle control, Trends Cell Biol 12, 104-7.

21. Hershko, A., and Ciechanover, A. (1998). The ubiquitin system, Annu Rev Biochem 67, 425-79.

22. Hershko, D., Bornstein, G., Ben-Izhak, O., Carrano, A., Pagano, M., Krausz, M. M., and Hershko, A. (2001). Inverse relation between levels of p27(Kip1) and of its ubiquitin ligase subunit Skp2 in colorectal carcinomas, Cancer 91, 1745-51.

23. Hsiung, Y. G., Chang, H. C., Pellequer, J. L., La Valle, R., Lanker, S., and Wittenberg, C. (2001). F-box protein Grr1 interacts with phosphorylated targets via the cationic surface of its leucine-rich repeat, Mol Cell Biol 21, 2506-20.

24. Huang, L., Kinnucan, E., Wang, G., Beaudenon, S., Howley, P. M., Huibregtse, J. M., and Pavletich, N. P. (1999). Structure of an E6AP-UbCH7 complex: insights into ubiquitination by the E2-E3 enzyme cascade, Science 286, 1321-6.

25. Hubbard, E. J., Wu, G., Kitajewski, J., and Greenwald, I. (1997). sel-10, a negative regulator of lin-12 activity in Caenorhabditis elegans, encodes a member of the CDC4 family of proteins, Genes Dev 11, 3182-93.

26. Jones, T. A., Zou, J. Y., Cowan, S. W., and Kjeldgaard, M. (1991). Improved methods for binding protein models in electron density maps and the localization of errors in these models. Acta Cryst. A47, 110-119.

27. Kaelin, W. G., Jr. (2002). Molecular basis of the VHL hereditary cancer syndrome. Nat. Rev. Cancer 2, 673-682.

28. Keyomarsi, K., Tucker, S. L., Buchholz, T. A., Callister, M., Ding, Y., Hortobagyi, G. N., Bedrosian, I., Knickerbocker, C., Toyofuku, W., Lowe, M., et al. (2002). Cyclin E and survival in patients with breast cancer. N. Engl. J. Med. 347, 1566-1575.

29. Koepp, D. M., Harper, J. W., and Elledge, S. J. (1999). How the cyclin became a cyclin: regulated proteolysis in the cell cycle, Cell 97, 431-4.

30. Koepp, D. M., Schaefer, L. K., Ye, X., Keyomarsi, K., Chu, C., Harper, J. W., and Elledge, S. J. (2001). Phosphorylation-dependent ubiquitination of cyclin E by the SCFFbw7 ubiquitin ligase, Science 294, 173-7.

31. Kominami, K., Ochotorena, I., and Toda, T. (1998). Two F-box/WD-repeat proteins Pop1 and Pop2 form hetero- and homo-complexes together with cullin-1 in the fission yeast SCF (Skp1-Cullin-1-F-box) ubiquitin ligase, Genes Cells 3, 721-35.

32. Lai, E. C. (2002). Protein degradation: four E3s for the notch pathway. Curr. Biol. 12, R74-78.

33. Lanker, S., Valdivieso, M. H., and Wittenberg, C. (1996). Rapid degradation of the G1 cyclin Cln2 induced by CDK-dependent phosphorylation, Science 271, 1597-601.

34. Lai, E. C. (2002). Protein degradation: four E3s for the notch pathway. Curr. Biol. 12, R74-78.

35. Lengronne, A., and Schwob, E. (2002). The yeast CDK inhibitor Sic1 prevents genomic instability by promoting replication origin licensing in late G1. Mol. Cell. 9, 1067-1078.

36. Mammen, M., Choi, S.-K., and Whitesides, G. M. (1998). Polyvalent interactions in biological systems: implications for design and use of multivalent ligands and inhibitors. Angew. Chem. 37, 2754-2794.

37. Maniatis, T. (1999). A ubiquitin ligase complex essential for the NF-kappaB, Wnt/Wingless, and hedgehog signaling pathways, Genes Dev 13, 505-510.

38. Margottin, F., Bour, S. P., Durand, H., Selig, L., Benichou, S., Richard, V., Thomas, D., Strebel, K., and Benarous, R. (1998). A novel human WD protein, h-beta TrCp, that interacts with HIV-1 Vpu connects CD4 to the ER degradation pathway through an F-box motif, Mol Cell 1, 565-74.

39. Meimoun, A., Holtzman, T., Weissman, Z., McBride, H. J., Stillman, D. J., Fink, G. R., and Kornitzer, D. (2000). Degradation of the transcription factor Gcn4 requires the kinase Pho85 and the SCF^{CDC4} ubiquitin-ligase complex. Mol. Biol. Cell. 11, 915-927.

40. Miller, R., Gallo, S. M., Khalak, H. G., and Weeks, C. M. (1994). SnB: crystal structure determination via Shake-and-Bake. J. Appl. Crystallgr. 27, 613-621.

41. Moberg, K. H., Bell, D. W., Wahrer, D. C., Haber, D. A., and Hariharan, I. K. (2001). Archipelago regulates Cyclin E levels in Drosophila and is mutated in human cancer cell lines, Nature 413, 311-6.

42. Nash, P., Tang, X., Orlicky, S., Chen, Q., Gertler, F. B., Mendenhall, M. D., Sicheri, F., Pawson, T., and Tyers, M. (2001). Multisite phosphorylation of a CDK inhibitor sets a threshold for the onset of DNA replication, Nature 414, 514-21.

43. Nicholls, A., Sharp, K. A., and Honig, B. (1991). Protein folding and association: insights from the interfacial and thermodynamic properties of hydrocarbons. Proteins Struct. Funct. Genet. 11, 281-296.

44. Nugroho, T. T., and Mendenhall, M. D. (1994). An inhibitor of yeast cyclin-dependent protein kinase plays an important role in ensuring the genomic integrity of daughter cells, Mol Cell Biol 14, 3320-3328.

45. Otwinowski, Z., and Minor, W. (1997). Processing of X-ray diffraction data collected in oscillation mode. Methods Enzymol. 276, 307-326.

46. Patton, E. E., Willems, A. R., and Tyers, M. (1998). Combinatorial control in ubiquitin-dependent proteolysis: don't Skp the F-box hypothesis, Trends Genet 14, 236-43.

47. Pawson, T., and Nash, P. (2000). Protein-protein interactions define specificity in signal transduction., Genes Dev 14, 1027-47.

48. Pickart, C. M. (2001). Mechanisms underlying ubiquitination, Annu Rev Biochem 70, 503-33.

49. Sakamoto, K. M., Kim, K. B., Kumagai, A., Mercurio, F., Crews, C. M., and Deshaies, R. J. (2001). Protacs: chimeric molecules that target proteins to the Skp1-Cullin-F box complex for ubiquitination and degradation, Proc Natl Acad Sci USA 98, 8554-9.

50. Schneider, B. L., Yang, Q. H., and Futcher, A. B. (1996). Linkage of replication to start by the Cdk inhibitor Sic1, Science 272, 560-2.

51. Schulman, B. A., Carrano, A. C., Jeffrey, P. D., Bowen, Z., Kinnucan, E. R., Finnin, M. S., Elledge, S. J., Harper, J. W., Pagano, M., and Pavletich, N. P. (2000). Insights into SCF ubiquitin ligases from the structure of the Skp1-Skp2 complex, Nature 408, 381-6.

52. Schwob, E., Bohm, T., Mendenhall, M. D., and Nasmyth, K. (1994). The B-type cyclin kinase inhibitor p40^{SIC1} controls the G1 to S transition in S. cerevisiae, Cell 79, 233-44.

53. Selkoe, D. J. (2001). Presenilin, Notch, and the genesis and treatment of Alzheimer's disease, Proc Natl Acad Sci USA 98, 11039-41.

54. Signoretti, S., Di Marcotulio, L., Richardson, A., Ramaswamy, S., Isaac, B., Rue, M., Monti, F., Loda, M., and Pagano, M. (2002). Oncogenic role of the ubiquitin ligase subunit Skp2 in human breast cancer, J Clin Invest 110, 633-41.

55. Skowyra, D., Craig, K. L., Tyers, M., Elledge, S. J., and Harper, J. W. (1997). F-box proteins are receptors that recruit phosphorylated substrates to the SCF ubiquitin-ligase complex, Cell 91, 209-19.

56. Sprague, E. R., Redd, M. J., Johnson, A. D., and Wolberger, C. (2000). Structure of the C-terminal domain of Tup1, a corepressor of transcription in yeast, Embo J 19, 3016-27.

57. Spruck, C. H., Strohmaier, H., Sangfelt, O., Muller, H. M., Hubalek, M., Muller-Holzner, E., Marth, C., Widschwendter, M., and Reed, S. 1. (2002). hCDC4 gene mutations in endometrial cancer, Cancer Res 62, 4535-9.

58. Spruck, C. H., Won, K. A., and Reed, S. I. (1999). Deregulated cyclin E induces chromosome instability, Nature 401, 297-300.

59. Strohmaier, H., Spruck, C. H., Kaiser, P., Won, K. A., Sangfelt, O., and Reed, S. I. (2001). Human F-box protein hCdc4 targets cyclin E for proteolysis and is mutated in a breast cancer cell line, Nature 413, 316-22.

60. Suzuki, H., Chiba, T., Suzuki, T., Fujita, T., Ikenoue, T., Omata, M., Furuichi, K., Shikama, H., and Tanaka, K. (2000). Homodimer of two F-box proteins beta TrCP1 or betaTrCP2 binds to IkappaBalpha for signal-dependent ubiquitination, J Biol Chem 275, 2877-84.

61. ter Haar, E., Harrison, S. C., and Kirchhausen, T. (2000). Peptide-in-groove interactions link target proteins to the beta-propeller of clathrin., Proc Natl Acad Sci USA 97, 1096-100.

62. Tong, A. H., Evangelista, M., Parsons, A. B., Xu, H., Bader, G. D., Page, N., Robinson, M., Raghibizadeh, S., Hogue, C. W., Bussey, H., et al. (2001). Systematic genetic analysis with ordered arrays of yeast deletion mutants, Science 294, 2364-8.

63. Tyers, M., and Jorgensen, P. (2000). Proteolysis and the cell cycle: with this RING I do thee destroy, Curr Opin Genet Dev 10, 54-64.

64. Varshavsky, A. (1991). Naming a targeting signal, Cell 64, 13-5.

65. Verdecia, M. A., Bowman, M. E., Lu, K. P., Hunter, T., and Noel, J. P. (2000). Structural basis for phosphoserine-proline recognition by group IV WW domains. Nat. Struct. Biol. 7, 639-643.

66. Verma, R., Annan, R. S., Huddleston, M. J., Carr, S. A., Reynard, G., and Deshaies, R. J. (1997). Phosphorylation of Sic1p by G1 Cdk required for its degradation and entry into S phase, Science 278, 455-460.

67. Wall, M. A., Coleman, D. E., Lee, E. I.-L., J. A., Posner, B. A., Gilman, A. G., and Sprang, S. R. (1995). The structure of the G protein heterotrimer G1 alpha 1 beta 1 gamma 2, Cell 83, 1047-58.

68. Wolf, D. A., McKeon, F., and Jackson, P. K. (1999). F-box/WD-repeat proteins Pop1p and Sud1p/Pop2p form complexes that bind and direct the proteolysis of Cdcl8p. Curr. Biol. 9, 373-376.

69. Wu, G., Hubbard, E. J., Kitajewski, J. K., and Greenwald, I. (1998). Evidence for functional and physical association between Caenorhabditis elegans SEL-10, a Cdc4p-related protein, and SEL-12 presenilin., Proc Natl Acad Sci USA 95, 15787-15891.

70. Wu, G., Lyapina, S., Das, I., Li, J., Gurney, M., Pauley, A., Chui, I., Deshaies, R. J., and Kitajewski, J. (2001). SEL-10 is an inhibitor of notch signaling that targets notch for ubiquitin-mediated protein degradation, Mol Cell Biol 21, 7403-15.

71. Yaffe, M. B., and Elia, A. E. (2001). Phosphoserine/threonine-binding domains, Curr Opin Cell Biol 13, 131-8.

72. Yaffe, M. B., Rittinger, K., Volinia, S., Caron, P. R., Aitken, A., Leffers, H., Gamblin, S. J., Smerdon, S. J., and Cantley, L. C. (1997). The structural basis for 14-3-3:phosphopeptide binding specificity, Cell 91, 961-71.

73. Zachariae, W., and Nasmyth, K. (1999). Whose end is destruction: cell division and the anaphase-promoting complex, Genes Dev 13, 2039-58.

74. Zheng, N., Schulman, B. A., Song, L., Miller, J. J., Jeffrey, P. D., Wang, P., Chu, C., Koepp, D. M., Elledge, S. J., Pagano, M., et al. (2002). Structure of the Cull-Rbx1-Skp1-F boxSkp2 SCF ubiquitin ligase complex, Nature 416, 703-9.

75. Zheng, N., Wang, P., Jeffrey, P. D., and Pavletich, N. P. (2000). Structure of a c-Cb1-UbCH7 complex: RING domain function in ubiquitin-protein ligases, Cell 102, 533-9.

1. An isolated binding pocket of a SCF complex or component thereof associated with substrate selection and/or orientation.

2. An isolated binding pocket of claim 1 wherein the component is an F-box protein comprising an F-box and a WD repeat domain.

3. Molecules or molecular complexes that comprise all or parts of one or more of a binding pocket as claimed in claim 1, or a homolog of the binding pocket that has similar structure and shape.

4. A crystal comprising a binding pocket of an F-box protein involved in substrate selection and/or orientation.

5. A crystal of claim 4 wherein the F box protein comprises an F box and a WD repeat domain.

6. A crystal comprising a binding pocket of claim 1 complexed or associated with a substrate.

7. A crystal of claim 6 wherein the ligand or substrate is a CPD motif containing protein, or part thereof.

8. A crystal according to claim 4 having the structural coordinates shown in Table 6.

9. A model of a binding pocket of a SCF complex using a crystal according to claim 8.

10. A model of: (a) a binding pocket of an SCF complex of claim 1; and (b) a modification of the model of (a).

11. A model of a binding pocket of an F-box protein of claim 2 that substantially represents the structural coordinates specified in Table 6.

12. A binding pocket of claim 2 which comprises a WD40 repeat domain characterized by one or more of the following characteristics:
(a) a 7 or 8 blade β-propeller structure, in particular a 8 blade β-propeller structure;
(b) a disk like structure characterized by a cavity in the middle and two opposing circular surfaces of different size;
(c) a conical frustum of about 40 Å top surface and about 50 Å bottom surface, an overall thickness of 30 Å and a central pore of 6 Å diameter; and
(d) a CPD binding site on the top surface of the frustum of (c) and running across the edge, while the bottom surface of the frustum links to the F-box domain.

13. A binding pocket of claim 2 which is characterized by one or more of the following characteristics:
(i) a pThr-Pro binding pocket;
(ii) a deep hydrophobic pocket that selects hydrophobic residues N-terminal to the phosphorylation site of a CPD motif, and
(iii) a through space electrostatic selection against basic residues C-terminal to the phosphorylation site of a CPD motif.

14. A binding pocket of claim 2 which comprises a helical linker characterized by a helices that form a stalk and pedestal like structure that connects and orients a WD repeat domain.

15. A binding pocket of claim 2 as shown in FIG. 3a which is further characterized by one or more of the following:
(a) a αhelix that is 30 Å in length and is anchored at its N-terminus to the hydrophobic core of an F-box/helical extension and at its C-terminus to the hydrophobic core of a WD repeat domain,
(b) the helix of (a) anchored at its amino terminus to an F-box through hydrophobic interactions;
(c) a second ahelix packed along the base of the helix of (a) or (b) opposite to the F-box through hydrophobic interactions; and
(d) a C-terminal end of the helix of (a) inserted obliquely between propeller blades β7 and β8 of an WD40 domain through van der Wals and hydrophobic interactions.

16. A binding pocket of claim 2 which is a CPD motif binding pocket comprising a hydrophobic pocket that surrounds the open central channel of a 7 or 8 blade WD repeat propeller.

17. A binding pocket of claim 2 which is a Cdc4 polypeptide that interacts with a CPD motif characterized by one or more of the following:
(a) a WD repeat domain surface composed of invariant and highly conserved residues from α-propeller blades;
(b) a three-sided pocket formed by Trp426, Thr386, and Arg 485;
(c) a three-sided pocket formed by Trp426, Thr441, Thr 465, and Arg 485;
(d) a hydrophobic pocket composed of Trp 426, Trp 717, Thr 386, and Val 384,
(e) a pocket formed by Leu634, Met590, and Tyr574; and
(f) a pocket formed by Arg485, Arg467, Arg534, Tyr548, and Arg572.

18. A binding pocket of claim 1 comprising one or more of the amino acid residues for an F-box protein crystal or F-box protein-substrate crystal identified in Table 3 or Table 4.

19. A computer-readable medium having stored thereon a crystal of claim 8.

20. A method of determining the secondary and/or tertiary structures of a polypeptide comprising the step of using a crystal of claim 8.

21. A method of screening for a ligand capable of associating with a binding pocket and/or inhibiting or enhancing the atomic contacts of interactions in a binding pocket, comprising the use of a crystal of claim 8.

22. A method of conducting a drug discovery business comprising:
(a) providing one or more systems employing the atomic interactions, atomic contacts, or structural coordinates of a binding pocket of claim 1, to identify agents by their ability to inhibit or potentiate the atomic interactions or atomic contacts of the binding pocket;
(b) conducting therapeutic profiling of agents identified in step (a), or further analogs thereof, for efficacy and toxicity in animals; and
(d) formulating a pharmaceutical preparation including one or more agents identified in step (b) as having an acceptable therapeutic profile.

23. A method for regulating an SCF complex by changing a structure of a binding pocket of claim 1.

24. Use of a modulator of a binding pocket of claim 1 in the manufacture of a medicament to treat and/or prevent a disease in a mammalian patient.

25. A pharmaceutical composition comprising a ligand or modulator of a binding pocket according to claim 1, and optionally a pharmaceutically acceptable carrier, diluent, excipient or adjuvant or any combination thereof.


SEQUENCE LISTING (43 sequences)

SEQ ID NO: 1 - protein, 8 aa, Homo sapiens
  misc_feature (1)..(1): wherein Xaa can be either Ile or Leu
  misc_feature (2)..(2): wherein Xaa can be either Ile, Leu or Pro
  MOD_RES (3)..(3): PHOSPHORYLATION
  misc_feature (5)..(5): wherein Xaa cannot be Lys or Arg
  misc_feature (6)..(6): wherein Xaa cannot be Lys or Arg
  misc_feature (7)..(7): wherein Xaa cannot be Lys or Arg
  misc_feature (8)..(8): wherein Xaa cannot be Lys or Arg
XXTPXXXX

SEQ ID NO: 2 - protein, 4 aa, Homo sapiens
  misc_feature (1)..(1): wherin Xaa may be Ser or Thr
  misc_feature (3)..(3): wherin Xaa can be any naturally occurring amino acid
  misc_feature (4)..(4): wherin Xaa can be Lys or Arg
XPXX

SEQ ID NO: 3 - protein, 194 aa, Saccharomyces cerevisiae
MVTSNVVLVSGEGERFTVDKKIAERSLLLKNYLNDMHDSNLQNNSDSESDSDSETNHKSKDNNNGDDDDEDDDEIVMPVPNVRSSVLQKVIEWAEHHRDSNFPDEDDDDSRKSAPVDSWDREFLKVDQEMLYEIILAANYLNIKPLLDAGCKVVAEMIRGRSPEEIRRTFNIVNDFTPEEEAAIRRENEWAEDR

SEQ ID NO: 4 - protein, 161 aa, Schizosaccharomyces pombe
MSKIKLISSDNEEFVVDQLIAERSMLIKNMLEDVGEINVPIPLPNVSSNVLRKVLEWCEHHKNDLYSGTEEESDIRLKKSTDIDEWDRKFMAVDQEMLFEIVLASNYLDIKPLLDTGCKTVANMIRGKSPEDIRKTFNIPNDFTPEEEEQIRKENEWAEDR

SEQ ID NO: 5 - protein, 165 aa, Caenorhabditis elegans
AKEREIKISSSDNEIFLVPRNVIRLSNTINTLLMDLGLDDEEGTNAEPIPVQNVTASILKKVISWCNHHHSDPISTEDSDNREKRTDDIGSWDVEFLKVDQGTLFELIAANYLDIKGLLDVTCKTVANMIKGKSPEEIRRTFNIKNDFTPEEEEQIRKENAWCED

SEQ ID NO: 6 - protein, 162 aa, Drosophila melanogaster
MPSIKLQSSDEEIFDTDIQIAKCSGTIKTMLEDCGMEDDENAIVPLPNVNSTILRKVLTWAHYHKDDPQPTEDDESKEKRTDDIISWDADFLKVDQGTLFELILAANYLDIKGLLELTCKTVANMIKGKTPEEIRKTFNIKKDFSPAEEEQVRKENEWCEEK

SEQ ID NO: 7 - protein, 163 aa, Homo sapiens
MPSIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWCEEK

SEQ ID NO: 8 - protein, 475 aa, Saccharomyces cerevisiae
LKRDLITSLPFEISLKIFNYLQFEDIINSLGVSQNWNKIIRKSTSLWKKLLISENFVSPKGFNSLNLKLSQKYPKLSQQDRLRLSFLENIFILKNWYNPKFVPQRTTLRGHMTSVITCLQFEDNYVITGADDKMIRVYDSINKKFLLQLSGHDGGVWALKYAHGGILVSGSTDRTVRVWDIKKGCCTHVFKGHNSTVRCLDIVEYKNIKYIVTGSRDNTLHVWKLPKESSVPDHGEEHDYPLVFHTPEENPYFVGVLRGHMASVRTVSGHGNIVVSGSYDNTLIVWDVAQMKCLYILSGHTDRIYSTIYDHERKRCISASMDTTIRIWDLENIWNNGECSYATNSASPCAKILGAMYTLQGHTALVGLLRLSDKFLVSAAADGSIRGWDANDYSRKFSYHHTNLSAITTFYVSDNILVSGSENQFNIYNLRSGKLVHANILKDADQIWSVNFKGKTLVAAVEKDGQSFLEILDFS

SEQ ID NO: 9 - protein, 475 aa, Schizosaccharomyces pombe
FQKNFLTGFPAEITNLVLTHLDAPSLCAVSQVSHHWYKLVSSNEELWKSLFLKDGFFWDSIDSKIRTMCLEQSLSACAIMKRVYFRHFNLRERWLHAPEKIKRCSFPIHGVRLITKLQFDDDKIIVSTCSPRINIYDTKTGVLIRSLEEHEGDVWTFEYVGDTLVTGSTDRTVRVWDLRTGECKQVFYGHTSTIRCIKIVQGNQSTTDTDDVEKENRPASNDANSMPPYIISSSRDCTIRLWSLPCLDDPPFVNVNENPDQNNDFTSATTNPFYIRTLRGHTDSVREVACLGDLIVSASYDGTLRVWKASTGVCLHVLRGHVGRVYSVTINPSRQQCISAGTDAKIRIWNLESGELLQTLHGHSNLVSQVTFNQNILVSASAPPDTSLRVWDLNTGSCRDILKCPLGHIFFQHDESKVVSGSHSTLQLWDIRSGKLVRDLLTDLDIIWQVAYNENVCVAAVLRNNRFWIEVLEFG

SEQ ID NO: 10 - protein, 465 aa, Caenorhabditis elegans
FQRDFLSCLPVELGMKILHNLTGYDLLKVAQVSKNWKLISEIDKIWKSLGVEEFKHHPDPTDRVTGAWQGTAIAAGVTIPDHIQPCDLNVHRFLKLQKFGDIFERAADKSRYLRADKIEKNWNANPIMGSAVLRGHEDHVITCMQIHDDVLVTGSDDNTLKVWCIDKGEVMYTLVGHTGGVWTSQISQCGRYIVSGSTDRTVKVWSTVDGSLLHTLQGHTSTVRCMAMAGSILVTGSRDTTLRVWDVESGRHLATLHGHHAAVRCVQFDGTTVVSGGYDFTVKIWNAHTGRCIRTLTGHNNRVYSLLFESERSIVCSGSLDTSIRVWDFTRPEGQECVALLQGHTSLTSGMQLRGNILVSCNADSHVRVWDIHEGTCVHMLSGHRSAITSLQWFGRNMVATSSDDGTVKLWDIERGALIRDLVTLDSGGNGGCIWRLCSTSTMLACAVGSRNNTEETKVILLDFD

SEQ ID NO: 11 - protein, 431 aa, Drosophila melanogaster
FQRDFISLLPRELALFVLSYLEPKDLLRAAQTCRSWRFLCDDNLLWKEKCRKAQILAEPRSDRPKRGRDGNMPPIASPWKAAYMRQHIIEMNWRSRPVRKPKVLKGHDDHVITCLQFSGNRIVSGSDDNTLKVWSAVNGKCLRTLVGHTGGVWSSQMSGNIIISGSTDRTLKVWDMDSGACVHTLQGHTSTVRCMHLHGSKVVSGSRDATLRVWDIEQGSCLHVLVGHLAAVRCVQYDGKLIVSGAYDYMVKIWHPERQECLHTLQGHTNRVYSLQFDGLHVVSGSLDTSIRVWDVETGNCKHTLMGHQSLTSGMELRQNILVSGNADSTVKVWDITTGQCLQTLSGPNKHHSAVTCLQFNSRFVVTSSDDGTVKLWDVKTGDFIRNLVALDSGGSGGVVWRIRANDTKLICAVGSRNGTEETKLMVLDFD

SEQ ID NO: 12 - protein, 428 aa, Homo sapiens
FQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFD

SEQ ID NO: 13 - protein, 424 aa, Caenorhabditis elegans
LQRDFISNLPAHLVELILFNVNSDSLKSCEEVSTSWRCALARGQHWKKLIEKNVRSDSLWWGLSEKRQWDKFLNISRDMSVRRICEKFNYDVNIKRDKLDQLILMHVFYSKLYPKIIRDIHNIDNNWKRGNYKMTRINCQSENSKGVYCLQYDDDKIVSGLRDNTIKIWDRKDYSCSRILSGHTGSVLCLQYDNRVIISGSSDATVRVWDVETGECIKTLIHHCEAVLHLRFANGIMVTCSKDRSIAVWDMVSPRDITIRRVLVGHRAAVNVVDFDDRYIVSASGDRTIKVWSMDTLEFVRTLAGHRRGIACLQYRGRLVVSGSSDNTIRLWDIHSGVCLRVLEGHEELVRCIRFDEKRIVSGAYDGKIKVWDLQAALDPRALSSEICLCSLVQHTGRVFRLQFDDFQIVSSSHDDTILIWDFL

SEQ ID NO: 14 - protein, 407 aa, Drosophila melanogaster
LQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRHMLRRINCRSENSKGVYCLQYDDGKIVSGLRDNTIKIWDRTDLQCVKTLMGHTGSVLCLQYDDKVIISGSSDSTVRVWDVNTGEMVNTLIHHCEAVLHLRFNNGMMVTCSKDRSIAVWDMTSPSEITLRRVLVGHRAAVNVVDFDEKYIVSASGDRTIKVWSTSSCEFVRTLNGHKRGIACLQYRDRLVVSGSSDNSIRLWDIECGACLRVLEGHEELVRCIRFDTKRIVSGAYDGKIKVWDLVAALDPRAASNTLCLNTLVEHTGRVFRLQFDEFQIVSSSHDDTILIWDFL

SEQ ID NO: 15 - protein, 408 aa, Homo sapiens
LQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFL

SEQ ID NO: 16 - protein, 459 aa, Saccharomyces cerevisiae
IKIDFISILPQELSLKILSYLDCQSLCNATRVCRKWQKLADDDRVWYHMCEQHIDRKCPNCGWGLPLLHMKRARIQQNSTGSSSNADIQTQTTRPWKVIYRERFKVESNWRKGHCRIQEFKGHMDGVLTLQFNYRLLFTGSYDSTIGIWDLFTGKLIRRLSGHSDGVKTLYFDDRKLITGSLDKTIRVWNYITGECISTYRGHSDSVLSVDSYQKVIVSGSADKTVKVWHVESRTCYTLRGHTEWVNCVKLHPKSFSCFSCSDDTTIRMWDIRTNSCLKVFRGHVGQVQKIIPLTIKDVENLATDNTSDGSSPQDDPTMTDGADESDTPSNEQETVLDENIPYPTHLLSCGLDNTIKLWDVKTGKCIRTQFGHVEGVWDIAADNFRIISGSHDGSIKVWDLQSGKCMHTFNGRRLQRETQHTQTQSLGDKVAPIACVCIGDSECFSGDEFGCVKMYKFD

SEQ ID NO: 17 - protein, 640 aa, Saccharomyces cerevisiae
  ref <308>: DATABASE ACCESSION NUMBER: Genbank/P39014
  ref <309>: DATABASE ENTRY DATE: 1995-02-01
  ref <313>: RELEVANT RESIDUES: (1)..(640)
MRRERQRMMSFEDKDKDDLDNSNSNNSSEMTDTAMMPPLKRLLITGSSDDLAQGSSGKKKMTMATRSPSSSPDLATNDSGTRVQPLPEYNFTKFCYRHNPDIQFSPTHTACYKQDLKRTQEINANIAKLPLQEQSDIHHIISKYSNSNDKIRKLILDGILSTSCFPQLSYISSLVTHMIKIDFISILPQELSLKILSYLDCQSLCNATRVCRKWQKLADDDRVWYHMCEQHIDRKCPNCGWGLPLLHMKRARIQQNSTGSSSNADIQTQTTRPWKVIYRERFKVESNWRKGHCRIQEFKGHMDGVLTLQFNYRLLFTGSYDSTIGIWDLFTGKLIRRLSGHSDGVKTLYFDDRKLITGSLDKTIRVWNYITGECISTYRGHSDSVLSVDSYQKVIVSGSADKTVKVWHVESRTCYTLRGHTEWVNCVKLHPKSFSCFSCSDDTTIRMWDIRTNSCLKVFRGHVGQVQKIIPLTIKDVENLATDNTSDGSSPQDDPTMTDGADESDTPSNEQETVLDENIPYPTHLLSCGLDNTIKLWDVKTGKCIRTQFGHVEGVWDIAADNFRIISGSHDGSIKVWDLQSGKCMHTFNGRRLQRETQHTQTQSLGDKVAPIACVCIGDSECFSGDEFGCVKMYKFDLND

SEQ ID NO: 18 - protein, 569 aa, Mus musculus
  ref <308>: DATABASE ACCESSION NUMBER: Genbank/NP_033901
  ref <309>: DATABASE ENTRY DATE: 1998-08-04
  ref <313>: RELEVANT RESIDUES: (1)..(569)
MDPAEAVLQEKALKFMNSSEREDCNNGEPPRKIIPEKNSLRQTYNSCARLCINQETVCLTSTAMKTENCVAKAKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDENAPPNSFYRALYPKIIQDIETIESNWRCGRHSLQRIHCRSETSKGVYCLQYDDQKIVSGLRDNTIKIWDKSTLECKRILTGHTGSVLCLQYDERVIITGSSDSTVRVWDVNAGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLMAALDPRAPAGTLCLRTLVEHSGRVFRLQFDEFQIVSSSHDDTILIWDFLNDPAAHAEPPRSPSRTYTYISR

SEQ ID NO: 19 - protein, 5 aa, Peptide Motif
  misc_feature (1)..(1): wherein Xaa can be Leu, Pro, or Ile
  DOMAIN (1)..(5): Cdc4 Phospho-Degron motif
  misc_feature (2)..(2): wherein Xaa can be any naturally occurring amino acid
  misc_feature (5)..(5): wherein Xaa can be any naturally occurring amino acid except basic and bulky hydrophobic amino acids
  misc_feature (5)..(5): wherein Xaa can be any naturally occurring amino acid
XXPTX

SEQ ID NO: 20 - protein, 8 aa, Peptide Motif
  misc_feature (1)..(1): wherein Xaa can be Leu, Pro, or Ile
  DOMAIN (1)..(8): Cdc4 Phospho-Degron motif
  misc_feature (2)..(2): wherein Xaa can be Leu, Ile, Val, or Pro
  misc_feature (5)..(5): wherein Xaa can be any naturally occurring amino acid except basic and bulky hydrophobic amino acids
  misc_feature (6)..(6): wherein Xaa can be any naturally occurring amino acid
  misc_feature (7)..(7): wherein Xaa can be any naturally occurring amino acid
  misc_feature (8)..(8): wherein Xaa can be any naturally occurring amino acid
XXTPXXXX

SEQ ID NO: 21 - protein, 4 aa, Peptide Motif
  misc_feature (1)..(1): wherein Xaa can be Leu, Gly or Thr
  DOMAIN (1)..(4): Cdc4 Phospho-Degron motif
XPTP

SEQ ID NO: 22 - protein, 779 aa, Saccharomyces cerevisiae
  ref <308>: DATABASE ACCESSION NUMBER: Genbank/S56245
  ref <309>: DATABASE ENTRY DATE: 1993-05-07
  ref <313>: RELEVANT RESIDUES: (1)..(51)
MGSFPLAEFPLRDIPVPYSYRVSGGIASSGSVTALVTAAGTHRNSSTAKTVETEDGEEDIDEYQRKRAAGSGESTPERSDFKRVKHDNHKTLHPVNLQNTGAASVDNDGLHNLTDISNDAEKLLMSVDDGSAAPSTLSVNMGVASHNVAAPTTVNAATITGSDVSNNVNSATINNPMEEGALPLSPTASSPGTTTPLAKTTKTINNNNNIADLIESKDSIISPEYLSDEIFSAINNNLPHAYFKNLLFRLVANMDRSELSDLGTLIKDNLKRDLITSLPFEISLKIFNYLQFEDIINSLGVSQNWNKIIRKSTSLWKKLLISENFVSPKGFNSLNLKLSQKYPKLSQQDRLRLSFLENIFILKNWYNPKFVPQRTTLRGHMTSVITCLQFEDNYVITGADDKMIRVYDSINKKFLLQLSGHDGGVWALKYAHGGILVSGSTDRTVRVWDIKKGCCTHVFKGHNSTVRCLDIVEYKNIKYIVTGSRDNTLHVWKLPKESSVPDHGEEHDYPLVFHTPEENPYFVGVLRGHMASVRTVSGHGNIVVSGSYDNTLIVWDVAQMKCLYILSGHTDRIYSTIYDHERKRCISASMDTTIRIWDLENIWNNGECSYATNSASPCAKILGAMYTLQGHTALVGLLRLSDKFLVSAAADGSIRGWDANDYSRKFSYHHTNLSAITTFYVSDNILVSGSENQFNIYNLRSGKLVHANILKDADQIWSVNFKGKTLVAAVEKDGQSFLEILDFSKASKINYVSNPVNSSSSSLESISTSLGLTRTTIIP

SEQ ID NO: 23 - protein, 684 aa, Candida albicans
  ref <308>: DATABASE ACCESSION NUMBER: Genbank/CAA65538
  ref <309>: DATABASE ENTRY DATE: 1996-04-04
  ref <313>: RELEVANT RESIDUES: (1)..(684)
MPSCDDTASSDTDCQSQVSSTAHLHSYRSNGLVEPPSKRRLTTTNETSLSSAGATTFQIESPGSISAITTNNSTTSAGDSNNSNSFSDQHSRHPRTPNAMNSPTHTPISDIEEDPIQQLPLPSPSASPIQSDTENEHVTTPDSLQGKANLDSIENVMSNEPTTQSELVDLVTKLSGFLSEANQNHLVFKLLQKTTRPTLSTFNNLINNSLKRDILSNVPFEVTMKILSYLDYKTLLSVAQVCKKWFDIINNPDTWIKLLKRDKLITDDAVIKYELQYPDQLLREWSTLPEINSAQVLYKKRKIIVNRWMDPKFKPHRISVSGHGNKVVTCLQHDDEKVVTGVDDKCISIYSTQTGQLMKVLEGHEGGVWALKYTGNTLVTGSTDRTVRVWNMKTGQCTHIFRGHTSTIRCLDIIHPAVIGKNQDGEDIVFPEYPLLITGSRDHNIHVWKLPVVDDSQDYIETFEGEFDNPYLIAVLSGHTQSVRSISGYGNIIISGSYDSTVRVWDLLDDGHCTHVLQGHLDRVYSTAIDFHSKTCFSGSMDSNINVWNFETGELKKVLVGHASLVGLLDLVDDVLVSAAADATLRIWDAKTGELRSKLKGHGAAITCFEHDGLRVVSGSEKMLKLWNVEKGTFARDLLSDVTGGIWQVRFDYKRCVAAVQRIINEDEGETFIEILDFSQPLLQ

SEQ ID NO: 24 - protein, 707 aa, Homo sapiens
  ref <308>: DATABASE ACCESSION NUMBER: Genbank/AAL07271
  ref <309>: DATABASE ENTRY DATE: 2001-08-30
  ref <313>: RELEVANT RESIDUES: (1)..(707)
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 25 - protein, 587 aa, Caenorhabditis elegans
  ref <308>: DATABASE ACCESSION NUMBER: Genbank/AAC47809
  ref <309>: DATABASE ENTRY DATE: 1997-12-12
  ref <313>: RELEVANT RESIDUES: (1)..(587)
MWPRNDVHMDDGSMTPEDQEPVTDNDMEYNDNGEESSYSNGSSSSYNADKLSSSRPLQHKLDLSASPSRNNDLNPRVEHLIALFKDLSSAEQMDAFTRLLQESNMTNIRQLRAIIEPHFQRDFLSCLPVELGMKILHNLTGYDLLKVAQVSKNWKLISEIDKIWKSLGVEEFKHHPDPTDRVTGAWQGTAIAAGVTIPDHIQPCDLNVHRFLKLQKFGDIFERAADKSRYLRADKIEKNWNANPIMGSAVLRGHEDHVITCMQIHDDVLVTGSDDNTLKVWCIDKGEVMYTLVGHTGGVWTSQISQCGRYIVSGSTDRTVKVWSTVDGSLLHTLQGHTSTVRCMAMAGSILVTGSRDTTLRVWDVESGRHLATLHGHHAAVRCVQFDGTTVVSGGYDFTVKIWNAHTGRCIRTLTGHNNRVYSLLFESERSIVCSGSLDTSIRVWDFTRPEGQECVALLQGHTSLTSGMQLRGNILVSCNADSHVRVWDIHEGTCVHMLSGHRSAITSLQWFGRNMVATSSDDGTVKLWDIERGALIRDLVTLDSGGNGGCIWRLCSTSTMLACAVGSRNNTEETKVILLDFDAVYP

SEQ ID NO: 26 - protein, 627 aa, Homo sapiens
  ref <308>: DATABASE ACCESSION NUMBER: Genbank/AAK57547
  ref <309>: DATABASE ENTRY DATE: 2001-08-22
  ref <313>: RELEVANT RESIDUES: (1)..(627)
MCVPRSGLILSCICLYCGVLLPVLLPNLPFLTCLSMSTLESVTYLPEKGLYCQRLPSSRTHGGTESLKGKNTENMGFYGTLKMIFYKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 27 - protein, 224 aa, Homo sapiens
MAVGNINELPENILLELFTHVPARQLLLNCRLVCSLWRDLIDLVTLWKRKCLREGFITEDWDQPVADWKIFYFLRSLHRNLLHNPCAEEGFEFWSLDVNGGDEWKVEDLSRDQRKEFPNDQVRSQARLRVQVPAVRSAPVVRARASGDLPARPGDHPAEERCQVEGGLPHILQLPARRPLHLVSARRRGHSLLGRLVRPEGHQQQHHHRAPAALTPPEPPSAEP

SEQ ID NO: 28 - protein, 815 aa, Saccharomyces cerevisiae
  ref <308>: DATABASE ACCESSION NUMBER: Genbank/AAB38821
  ref <309>: DATABASE ENTRY DATE: 1996-12-17
  ref <313>: RELEVANT RESIDUES: (1)..(815)
MSETLPRSDDLEATWNFIEPGINQILGNEKNQASTSKRVYKILSPTMYMEVYTAIYNYCVNKSRSSGHFSTDSRTGQSTILVGSEIYEKLKNYLKNYILNFKQSNSETFLQFYVKRWKRFTIGAIFLNHAFDYMNRYWVQKERSDGKRHIFDVNTLCLMTWKEVMFDPSKDVLINELLDQVTLGREGQIIQRSNISTAIKSLVALGIDPQDLKKLNLNVYIQVFEKPFLKKTQEYYTQYTNDYLEKHSVTEYIFEAHEIIKREEKAMTIYWDDHTKKPLSMALNKVLITDHIEKLENEFVVLLDARDIEKITSLYALIRRDFTLIPRMASVFENYVKKTGENEISSLLAMHKHNIMKNENANPKKLALMTAHSLSPKDYIKKLLEVHDIFSKIFNESFPDDIPLAKALDNACGAFININEFALPAGSPKSATSKTSEMLAKYSDILLKKATKPEVASDMSDEDIITIFKYLTDKDAFETHYRRLFAKRLIHGTSTSAEDEENIIQRLQAANSMEYTGKITKMFQDIRLSKILEDDFAVALKNEPDYSKAKYPDLQPFVLAENMWPFSYQEVEFKLPKELVPSHEKLKESYSQKHNGRILKWLWPLCRGELKADIGKPGRMPFNFTVTLFQMAILLLYNDADVLTLENIQEGTSLTIQHIAAAMVPFIKFKLIQQVPPGLDALVKPETQFKLSRPYKALKTNINFASGVKNDILQSLSGGGHDNHGNKLGNKRLTEDERIEKELNTERQIFLEACIVRIMKAKRNLPHTTLVNECIAQSHQRFNAKVSMVKRAIDSLIQKGYLQRGDDGESYAYLA

SEQ ID NO: 29 - protein, 768 aa, Homo sapiens
  ref <308>: DATABASE ACCESSION NUMBER: Genbank/AAC36304
  ref <309>: DATABASE ENTRY DATE: 1998-09-19
  ref <313>: RELEVANT RESIDUES: (1)..(768)
MSNLSKGTGSRKDTKMRIRAFPMTMDEKYVNSIWDLLKNAIQEIQRKNNSGLSFEELYRNAYTMVLHKHGEKLYTGLREVVTEHLINKVREDVLNSLNNNFLQTLNQAWNDHQTAMVMIRDILMYMDRVYVQQNNVENVYNLGLIIFRDQVVRYGCIRDHLRQTLLDMIARERKGEVVDRGAIRNACQMLMILGLEGRSVYEEDFEAPFLEMSAEFFQMESQKFLAENSASVYIKKVEARINEEIERVMHCLDKSTEEPIVKVVERELISKHMKTIVEMENSGLVHMLKNGKTEDLGCMYKLFSRVPNGLKTMCECMSSYLREQGKALVSEEGEGKNPVDYIQGLLDLKSRFDRFLLESFNNDRLFKQTIAGDFEYFLNLNSRSPEYLSLFIDDKLKKGVKGLTEQEVETILDKAMVLFRFMQEKDVFERYYKQHLARRLLTNKSVSDDSEKNMISKLKTECGCQFTSKLEGMFRDMSISNTTMDEFRQHLQATGVSLGGVDLTVRVLTTGYWPTQSATPKCNIPPAPRHAFEIFRRFYLAKHSGRQLTLQHHMGSADLNATFYGPVKKEDGSEVGVGGAQVTGSNTRKHILQVSTFQMTILMLFNNREKYTFEEIQQETDIPERELVRALQSLACGKPTQRVLTKEPKSKEIENGHIFTVNDQFTSKLHRVKIQTVAAKQGESDPERKETRQKVDDDRKHEIEAAIVRIMKSRKKMQHNVLVAEVTQQLKARFLPSPVVIKKRIEGLIEREYLARTPEDRKVYTYVA

SEQ ID NO: 30 - protein, 745 aa, Homo sapiens
  ref <308>: DATABASE ACCESSION NUMBER: Genbank/ AAC51190
  ref <309>: DATABASE ENTRY DATE: 1997-04-03
  ref <313>: RELEVANT RESIDUES: (1)..(745)
MSLKPRVVDFDETWNKLLTTIKAVVMLEYVERATWNDRFSDIYALCVAYPEPLGERLYTETKIFLENHVRHLHKRVLESEEQVLVMYHRYWEEYSKGADYMDCLYRYLSTQFIKKNKLTEADLQYGYGGVDMNEPLMEIGELALDMWRKLMVEPLQAILIRMLLREIKNDRGGEDPNQKVIHGVINSFVHVEQYKKKFPLKFYQEIFESPFLTETGEYYKQEASNLLQESNCSQYMEKVLGRLKDEEIRCRKYLHPSSYTKVIHECQQRMVADHLQFLHAECHNIIRQEKKNDMANMYVLLRAVSTGLPHMIQELQNHIHDEGLRATSNLTQENMPTLFVESVLEVHGKFVQLINTVLNGDQHFMSALDKALTSVVNYREPKSVCKAPELLAKYCDNLLKKSAKGMTENEVEDRLTSFITVFKYIDDKDVFQKFYARMLAKRLIHGLSMSMDSEEAMINKLKQACGYEFTSKLHRMYTDMSVSADLNNKFNNFIKNQDTVIDLGISFQIYVLQAGAWPLTQAPSSTFAIPQELEKSVQMFELFYSQHFSGRKLTWLHYLCTGEVKMNYLGKPYVAMVTTYQMAVLLAFNNSETVSYKELQDSTQMNEKELTKTIKSLLDVKMINHDSEKEDIDAESSFSLNMNFSSKRTKFKITTSMQKDTPQEMEQTRSAVDEDRKMYLQAAIVRIMKARKVLRHNALIQEVISQSRARFNPSISMIKKCIEVLIDKQYIERSQASADEYSYVA

SEQ ID NO: 31 - protein, 768 aa, Homo sapiens; identical to SEQ ID NO: 29

SEQ ID NO: 32 - protein, 745 aa, Homo sapiens
  ref <308>: DATABASE ACCESSION NUMBER: Genbank/ AAD23581
  ref <309>: DATABASE ENTRY DATE: 1999-04-06
  ref <313>: RELEVANT RESIDUES: (1)..(745)
MSLKPRVVDFDETWNKLLTTIKAVVMLEYVERATWNDRFSDIYALCVAYPEPLGERLYTETKIFLENHVRHLHKRVLESEEQVLVMYHRYWEEYSKGADYMDCLYRYLNTQFIKKNKLTEADLQYGYGGVDMNEPLMEIGELALDMWRKLMVEPLQAILIRMLLREIKNDRGGEDPNQKVIHGVINSFVHVEQYKKKFPLKFYQEIFESPFLTETGEYYKQEASNLLQESNCSQYMEKVLGRLKDEEIRCRKYLHPSSYTKVIHECQQRMVADHLQFLHAECHNIIRQEKKNDMANMYVLLRAVSTGLPHMIQELQNHIHDEGLRATSNLTQENMPTLFVESVLEVHGKFVQLINTVLNGDQHFMSALDKALTSVVNYREPKSVCKAPELLAKYCDNLLKKSAKGMTENEVEDRLTSFITVFKYIDDKDVFQKFYARMLAKRLIHGLSMSMDSEEAMINKLKQACGYEFTSKLHRMYTDMSVSADLNNKFNNFIKNQDTVIDLGISFQIYVLQAGAWPLTQAPSSTFAIPQELEKSVQMFELFYSQHFSGRKLTWLHYLCTGEVKMNYLGKPYVAMVTTYQMAVLLAFNNSETVSYKELQDSTQMNEKELTKTIKSLLDVKMINHDSEKEDIDAESSFSLNMNFSSKRTKFKITTSMQKDTPQEMEQTRSAVDEDRKMYLQAAIVRIMKARKVLRHNALIQEVISQSRARFNPSISMIKKCIEVLIDKQYIERSQASADEYSYVA

SEQ ID NO: 33 - protein, 770 aa, Caenorhabditis elegans
CDSEVVWKKLQDGLDVAYRRENMAPKDYMTLYTSVYDYCTSITLSTSRRDGEDGRAESSTPARTAGADFVGHEMYQRVEEYVKAYVIAVCEKGAELSGEDLLKYYTTEWENFRISSKVMDGIFAYLNRHWIRRELDEGHENIYMVYTLALVVWKRNLFNDLKDKVIDAMLELIRSERTGSMINSRYISGVVECLVELGVDDSETDAKKDAETKKLAVYKEFFEVKFLEATRGFYTQEAANFLSNGGNVTDYMIKVETRLNQEDDRCQLYLNSSTKTPLATCCESVLISNQLDFLQRHFGGLLVDKRDDDLSRMFKLCDRVPNGLDELRKSLENHIAKEGHQALERVAMEAATDAKLYVKTLLEVHERYQSLVNRSFKNEPGFMQSLDKAATSFINNNAVTKRAPPQAQLTKSAELLARYCDQLLRKSSKMPDEAELEELQTKIMVVFKYIDDKDVFSKFYTKMFSKRLISELSASDEAEANFITKLKSMCGYEYTARLSKMVNDTQVSKDLTADFKEKKADMLGQKSVEFNVLVLSSGSWPTFPTTPITLPQQLSKTIEIFGQFYNEKFNGRRLTWVYSQSRGEITSTAFPKKYVFTATTAQMCTMLLFNEQDSYTVEQIAAATKMDEKSAPAIVGSLIKNLVLKADTELQKEDEVPMTATVSLNKAYMNKKVRVDLSKFTMKQDAVRDTENVQKNVEEDRKSVISACIVRIMKTRKRVQHQQLMTEVITQLSGRFKPKVEMIKRCIGSLIEKEYMLRTEGQKDLYEYLA

SEQ ID NO: 34 - protein, 773 aa, Drosophila melanogaster
  ref <308>: DATABASE ACCESSION NUMBER: Genbank/AAA85085
  ref <309>: DATABASE ENTRY DATE: 1996-01-03
  ref <313>: RELEVANT RESIDUES: (1)..(773)
MNRSGNSQTTQKLVNLDDIWSELVEGIMQVFEHEKSLTRSQYMRFYTHVYDYCTSVSAAPSGRSSGKTGGAQLVGKKLYDRLEQFLKSYLSELLTKFKAISGEEVLLSRYTKQWKSYQFSSTVLDGICNYLNRNWVKRECEEGQKGIYKIYRLALVAWKGHLFQVLNEPVTKAVLKSIEEERQGKLINRSLVRDVIECYVELSFNEEDTDAEQQKLSVYKQNFENKFIADTSAFYEKESDAFLSTNTVTEYLKHVENRLEEETQRRGFNSKNGLSYLHETTADVLKSTCEEVLIEKHLKIFHTEFQNLLNADRNDDLKRMYSLVALSSKNLTDLKSILENHILHQGTEAIAKCCTTDAANDPKTYVQTILDVHKKYNALVLTAFNNDNGFVAALDKACGKFINSNVVTIANSASKSPELLAKYCDLLLKKSSKNPEDKELEDNLNQVMVVFKYIEDKDVFQKYYSKMLAKRLVNHTSASDDAEAMMISKLKQTCGYEYTVKLQRMFQDIGVSKDLNSYFKQYLAEKNLTMEIDFGIEVLSSGSWPFQLSNNFLLPSELERSVRQFNEFYAARHSGRKLNWLYQMCKGELIMNVNRNNSSTYTLQASTFQMSVLLQFNDQLSFTVQQLQDNTQTQQENLIQVLQILLKAKVLTSSDNENSLTPESTVELFLDYKNKKRRININQPLKTELKVEQETVHKHIEEDRKLLIQAAIVRIMKMRKRLNHTNLISEVLNQLSTRFKPKVPVIKKCIDILIEKEYLERMEGHKDTYSYLA

SEQ ID NO: 35 - protein, 387 aa, Schizosaccharomyces pombe
  ref <308>: DATABASE ACCESSION NUMBER: Q10452
  ref <309>: DATABASE ENTRY DATE: 2003-09-15
  ref <313>: RELEVANT RESIDUES: (1)..(387)
MNHGTKIPVDPFRIIKETARDGSTGEVKQLSYTSSKVVGSGSFGVVMQVHLIESDSKAAIKRVLQDKRFKNRELQIMRIMKHPNIVDLIAYYYTTGDNSDEVYLNLVLEFMPETIYRASRLYTRQKLSMPMLEVKLYIYQLLRSLAYIHASGICHRDIKPQNLLLDPENGILKLCDFGSAKILVAGEPNVSYICSRYYRAPELIFGATDYTHAIDIWSTGCVMAELMLGHPLFPGESGIDQLVEIIKILGTPSREQIKTMNPNYMEHRFPQIRPQPLSRVFSRSVPLDALDLLSKMLQYTPTDRLTAAEAMCHPFFDELRDPNTKLHNSRNPDASPRHLPELFNFSPFELSIRPDLNQKLIPSHARDALPVKLDDFVPIPIHRARID

SEQ ID NO: 36 - protein, 161 aa, Schizosaccharomyces pombe; identical to SEQ ID NO: 4
  ref <308>: DATABASE ACCESSION NUMBER: BAB62325
  ref <309>: DATABASE ENTRY DATE: 2001-08-01
  ref <313>: RELEVANT RESIDUES: (1)..(161)

SEQ ID NO: 37 - protein, 194 aa, Saccharomyces cerevisiae
  ref <308>: DATABASE ACCESSION NUMBER: Genbank/AAC49492
  ref <309>: DATABASE ENTRY DATE: 1996-10-25
  ref <313>: RELEVANT RESIDUES: (1)..(194)
MVTSNVVLVSGEGERFTVDKKIAERSLLLKNYLNDMHDSNLQNNSDSDSDSDSETNHKSKDNNNGDDDDEDDDEIVMPVPNVRSSVLQKVIEWAEHHRDSNFPDEDDDDSRKSAPVDSWDREFLKVDQEMLYEIILAANYLNIKPLLDAGCKVVAEMIRGRSPEEIRRTFNIVNDFTPEEEAAIRRENEWAEDR

SEQ ID NO: 38 - protein, 194 aa, Saccharomyces cerevisiae; identical to SEQ ID NO: 3
  ref <308>: DATABASE ACCESSION NUMBER: Genbank/AAB17500
  ref <309>: DATABASE ENTRY DATE: 1996-10-25
  ref <313>: RELEVANT RESIDUES: (1)..(194)

SEQ ID NO: 39 - DNA, 9 bp, artificial
  Insert into coding sequence of CDC4
ggcgaactg

SEQ ID NO: 40 - protein, 9 aa, Peptide Motif
  MOD_RES (1)..(1): ACETYLATION
  DOMAIN (1)..(9)
  MOD_RES (4)..(4): PHOSPHORYLATION
  MOD_RES (9)..(9): AMIDATION
GLLTPPQSG

SEQ ID NO: 41 - protein, 4 aa, Peptide Motif
  DOMAIN (1)..(4)
  MOD_RES (2)..(2): PHOSPHORYLATION
LTPP

SEQ ID NO: 42 - protein, 6 aa, Peptide Motif
  DOMAIN (1)..(6)
  MOD_RES (2)..(2): PHOSPHORYLATION
  misc_feature (4)..(4): wherein Xaa can be any naturally occurring amino acid
  misc_feature (5)..(5): wherein Xaa can be any naturally occurring amino acid
  MOD_RES (6)..(6): PHOSPHORYLATION
DSGXXS

SEQ ID NO: 43 - protein, 9 aa, Peptide Motif
  DOMAIN (1)..(9)
  MOD_RES (4)..(4): PHOSPHORYLATION
FLPTPVLED